(19) United States
(12) Patent Application Publication
(10) Pub. No.: US 2022/0220561 A1
(43) Pub. Date: Jul. 14, 2022

(54) METHODS AND CLINICAL PROTOCOLS AND KITS PERTAINING TO MAKING AND USING THERAPEUTIC COMPOSITIONS FOR CELLULAR TREATMENT

(71) Applicant: ADVANCED REGEN MEDICAL TECHNOLOGIES, LLC, Houston, TX (US)

(72) Inventors: Steven John GRECO, Houston, TX (US); Khadidiatou GUIRO, Houston, TX (US)

(21) Appl. No.: 17/438,623

(22) PCT Filed: Mar. 16, 2020
(86) PCT No.: PCT/US2020/023011

§ 371 Date: Sep. 13, 2021

Related U.S. Application Data
(60) Provisional application No. 62/819,972, filed on Mar. 18, 2019.
(60) Provisional application No. 62/820,168, filed on Mar. 18, 2019.
(60) Provisional application No. 62/850,371, filed on May 20, 2019.

(51) Int. Cl. C12Q 1/6886; C12N 5/0786; C12N 1/04; A61K 35/17

(52) U.S. Cl. CPC C12Q 1/6886; C12N 5/0645; C12Q 2600/158; A61K 35/17; C12N 1/04

(57) ABSTRACT

Disclosed herein are methods for preparing clinically useable target cells for use in achieving a clinical effect in patients. The protocols disclosed herein have been shown to improve cell yield during collection of target cells, transport of target cells, storage of target cells, and use of target cells. Also disclosed herein are kits and methods for testing patients and preparing clinically useable target cells for use in achieving a clinical effect in patients. The kits and protocols therein improve cell yield during collection of target cells, transport of target cells, storage of target cells, and use of target cells.

### RELATED APPLICATIONS

This application claims the benefit of U.S. Provisional Application Ser. No. 62/819,972, filed Mar. 18, 2019, U.S. Provisional Application Ser. No. 62/820,168, filed Mar. 18, 2019, and U.S. Provisional Application Ser. No. 62/850,371, filed May 20, 2019, each of which are hereby incorporated by reference in its entirety.

### REFERENCE TO SEQUENCE LISTING

The present application is being filed along with a Sequence Listing in electronic format. The Sequence Listing is provided as a file entitled SeqListingARGMT011WO.TXT, created and last saved on Mar. 16, 2020, which is 97,343 bytes in size. The information in the electronic format of the Sequence Listing is hereby incorporated by reference in its entirety.

### FIELD

Disclosed herein are compounds, compositions, methods of making thereof, and methods for the treatment of disease using the same and for the improvement of one or more aspects of cellular function through the use of the same. Some embodiments pertain to methods of treating patients and clinical protocols for doing the same, including manufacturing target cells for use in treatment. Also disclosed herein are kits including one or more instructions, containers, compounds, compositions, and methods for collecting blood, blood products, cells, and/or treating cells. In some embodiments, the kits provide instructions and devices/tools for the treatment of disease and for the improvement of one or more aspects of cellular function, including kits for use in the methods disclosed herein.

### BACKGROUND

Dysfunctional and/or senescent cellular signaling is an important risk factor for most chronic diseases and is a primary factor for the majority of morbidity and health care expenditures in developed nations.

### SUMMARY

Disclosed herein are methods for preparing clinically useable target cells for use in achieving a clinical effect in patients. The protocols disclosed herein have been shown to improve cell yield during collection of target cells, transport of target cells, storage of target cells, and use of target cells.

In some embodiments, at least one target cell for use in treating a patient with cellular dysfunctional or an age-related disorder is prepared. In some embodiments, at least one donor cell from a donor is provided. In some embodiments, at least one patient cell from a patient is provided. In some embodiments, the patient cell is exposed to the donor cell in an environment that is substantially-free of animal-based factors to provide at least one target cell. In some embodiments, the donor is younger than the patient. In some embodiments, the donor cell is exposed to the subject cell in a manner that prevents the donor cell and the subject cell from becoming mixed. In some embodiments, the donor cell is provided as a cryogenically frozen donor cell that is thawed prior to exposure to the subject cell. In some embodiments, the subject cell is provided as a cryogenically frozen subject cell that is thawed prior to exposure to the donor cell.

In some embodiments, the at least one target cell for use in treating a patient with cellular dysfunctional or an age-related disorder may be provided using one or more of the following steps in addition to or instead of the steps recited above. In some embodiments, at least one patient cell from the patient is provide. In some embodiments, the patient cell is contacted with one or more interfering RNA(s) (RNAi(s)) selected from SEQ ID NOs:9-20 and/or one or more small molecule drugs that inhibit PAX5 and/or PPM1F (as disclosed elsewhere herein) in an environment that is substantially-free of animal-based factors to provide a target cell. In some embodiments, the patient is exposed to the target cell thereby treating the patient. In some embodiments, the patient cell is provided as a cryogenically frozen patient cell that is thawed prior to contact with the interfering RNA(s) and/or small molecule drugs. In some embodiments, the target cell is provided as a cryogenically frozen target cell that is thawed prior to administration to the patient.

In some embodiments, the at least one target cell for use in treating a patient with cellular dysfunctional or an age-related disorder may be provided using one or more of the following steps in addition to or instead of the steps recited above. In some embodiments, at least one patient cell from the patient is provided. In some embodiments, the patient cell is contacted with one or more interfering RNA(s) (RNAi(s)) having at least 80% identity to one or more of SEQ ID NOs:9-20 in an environment that is substantially-free of animal-based factors to provide a target cell. In some embodiments, the patient cell is contacted with one or more interfering RNA(s) (RNAi(s)) selected from one or more sequences having not more than 1, 2, or 3, amino acid substitutions or insertions to one or more of SEQ ID NOs:9-20 in an environment that is substantially-free of animal-based factors to provide a target cell. In some embodiments, the patient is exposed to the target cell thereby treating the patient. In some embodiments, patient cell is provided as a cryogenically frozen patient cell that is thawed prior to contact with the interfering RNA(s). In some embodiments, the target cell is provided as a cryogenically frozen target cell that is thawed prior to administration to the patient.

Some embodiments pertain to the target cell made by the method recited above or elsewhere herein. Some embodiments pertain to a pharmaceutical composition comprising the target cell.

Some embodiments pertain to a method of treating a patient with cellular dysfunctional or an age-related disorder. In some embodiments, at least one donor cell from a donor is provided. In some embodiments, at least one patient cell from a patient is provided. In some embodiments, the patient cell is exposed to the donor cell in an environment that is substantially-free of animal-based factors to provide at least one target cell. In some embodiments, the patient is exposed to the target cell thereby treating the patient. In some embodiments, the donor is younger than the patient and/or wherein the donor is that patient at a younger age. In some embodiments, the donor cell is exposed to the subject cell in a manner that prevents the donor cell and the subject cell from becoming mixed. In some embodiments, the donor cell is provided as a cryogenically frozen donor cell that is thawed prior to exposure to the subject cell. In some embodiments, the subject cell is provided as a cryogenically frozen subject cell that is thawed prior to exposure to the donor cell.

In some embodiments, at least one patient cell from the patient. In some embodiments, the patient cell is contacted with one or more interfering RNA(s) (RNAi(s)) selected from SEQ ID NOs:9-20 and/or one or more small molecule drugs that inhibit PAX5 and/or PPM1F to provide a target cell. In some embodiments, the patient is exposed to the target cell thereby treating the patient. In some embodiments, the patient cell is contacted with one or more interfering RNA(s) (RNAi(s)) having at least 80% identity to one or more of SEQ ID NOs:9-20 to provide a target cell. In some embodiments, the patient to is exposed to the target cell thereby treating the patient. In some embodiments, the patient cell is provided as a cryogenically frozen patient cell that is thawed prior to contact with the interfering RNA(s) and/or small molecule drugs. In some embodiments, the target cell is provided as a cryogenically frozen target cell that is thawed prior to administration to the patient. In some embodiments, the patient to the one or more small molecule drugs.

In some embodiments, at least one donor cell is provided from a donor. In some embodiments, at least one subject cell is provided from a subject. In some embodiments, at least one patient cell is provided from a patient. In some embodiments, the subject cell is exposed to the donor cell in an environment that is substantially-free of animal-based factors to provide at least one intermediate cell. In some embodiments, the patient cell is exposed to the intermediate cell in an environment that is substantially-free of animal-based factors to provide at least one target cell. In some embodiments, the patient is exposed to the target cell thereby treating the patient. In some embodiments, the patient cell is contacted with one or more RNAi(s) selected from SEQ ID NOs:9-20 and/or one or more small molecule drugs that inhibit PAX5 and/or PPM1F in an environment that is substantially-free of animal-based factors to provide a target cell. In some embodiments, the patient and donor are related by consanguinity. In some embodiments, the one or more small molecule drugs is a polycyclic aromatic compound that antagonize a PAX5 protein and/or PP1F protein or reduce the expression of a PAX5 gene and/or a PPM1F gene.

In some embodiments, the polycyclic aromatic compound is of formula I:

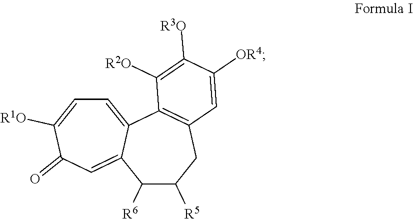

wherein each of R₁, R₂, R₃, R₄, R₅, and R₆ is independently selected from —H, hydroxyl, halogen, C₁ to C₆ alkyl optionally substituted with halogen or hydroxy, optionally substituted C₁ to C₆ alkenyl, optionally substituted C₁ to C₆ alkynyl, optionally substituted C₁ to C₆ alkoxy, optionally substituted C₁ to C₆ haloalkyl, optionally substituted C₁ to C₆ haloalkoxy, mono-substituted amine(C₁ to C₆ alkyl optionally substituted), a di-substituted amine(C₁ to C₆ alkyl optionally substituted), a diamino-group, and an optionally substituted polyether—having 1 to 6 repeat units. In some embodiments, each of R₁, R₂, R₃, R₄, R₅, and R₆ is independently selected from —H, hydroxyl, halogen, C₁ to C₆ alkyl optionally substituted with halogen or hydroxy, and a —(OR_{B}—)ₒOH, where R_{B} is an optionally substituted C₁ to C₆ alkyl.

In some embodiments, the polycyclic compound is of formula II:

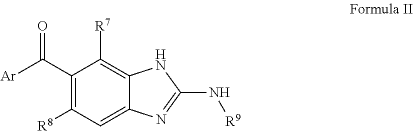

wherein each of R₇, R₈, and R₉ is independently selected from —H, hydroxyl, halogen, C₁ to C₆ alkyl optionally substituted with halogen or hydroxy, optionally substituted C₁ to C₆ alkenyl, optionally substituted C₁ to C₆ alkynyl, optionally substituted C₁ to C₆ alkoxy, optionally substituted C₁ to C₆ haloalkyl, optionally substituted C₁ to C₆ haloalkoxy, mono-substituted amine(C₁ to C₆ alkyl optionally substituted), a di-substituted amine(C₁ to C₆ alkyl optionally substituted), a diamino-group, and an optionally substituted polyether—having 1 to 6 repeat units. In some embodiments, each of R₇, R₈, and R₉ is independently selected from —H, hydroxyl, halogen, C₁ to C₆ alkyl optionally substituted with halogen or hydroxy, and a —(OR_{B}—)ₒOH, where R_{B} is an optionally substituted C₁ to C₆ alkyl.

In some embodiments, the polycyclic compound is of formula III:

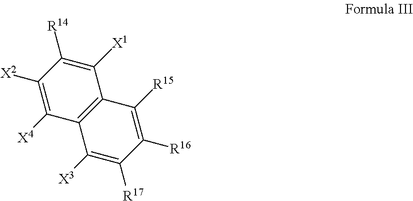

wherein each of X₁, X₂, X₃, X₄ is independently selected from —H, hydroxyl, halogen, —NH₂, optionally substituted —SO₂OR₁₈; and wherein each of R₁₄, R₁₅, R₁₆, R₁₇, and R₁₈ is independently selected from —H, hydroxyl, halogen, —NH₂, C₁ to C₆ alkyl optionally substituted with halogen or hydroxy, optionally substituted C₁ to C₆ alkenyl, optionally substituted C₁ to C₆ alkynyl, optionally substituted C₁ to C₆ alkoxy, optionally substituted C₁ to C₆ haloalkyl, optionally substituted C₁ to C₆ haloalkoxy, mono-substituted amine(C₁ to C₆ alkyl optionally substituted), a di-substituted amine(C₁ to C₆ alkyl optionally substituted), a diamino-group, and an optionally substituted polyether—having 1 to 6 repeat units. In some embodiments, R₁₄, R₁₅, R₁₆, R₁₇, and R₁₈ are independently selected from —H, hydroxyl, halogen, C₁ to C₆ alkyl optionally substituted with halogen or hydroxy, and a —(OR_{B}—)ₒOH, where R_{B} is an optionally substituted C₁ to C₆ alkyl.

In some embodiments, the compound of formula III is represented by the following structure:

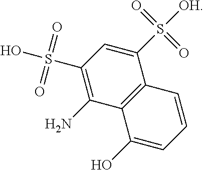 [SMILES: Nc1c(S(=O)(=O)O)cc(S(=O)(=O)O)c2cccc(O)c12]

In some embodiments, the polycyclic compound is selected from the group consisting of:

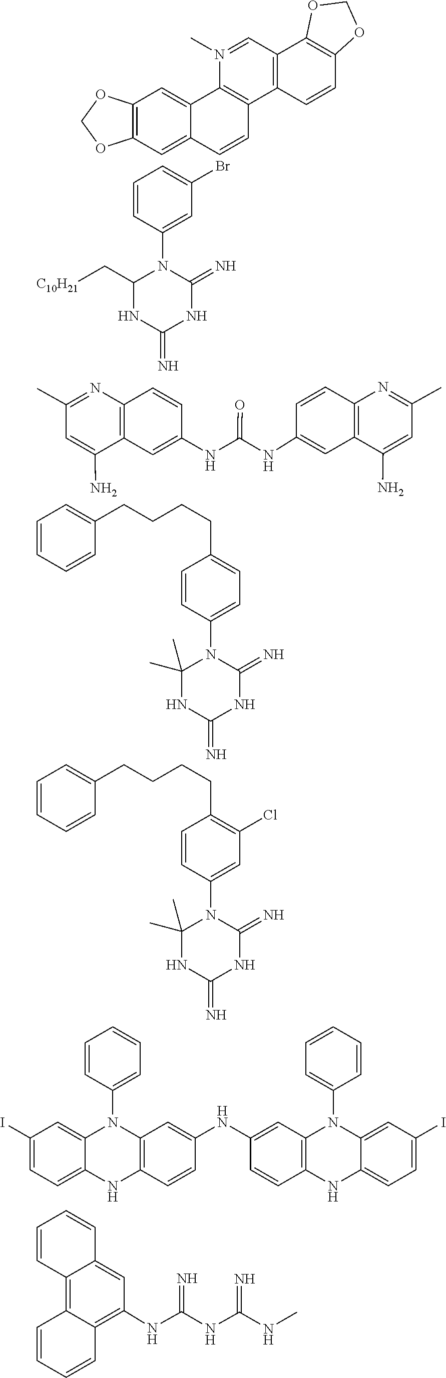

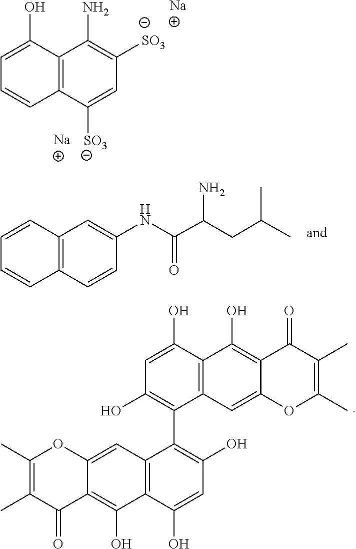

In some embodiments, the polycyclic compound is provided as a pharmaceutically acceptable salt.

In some embodiments of the above methods (or methods disclosed elsewhere herein), the cellular dysfunctional or age-related disorder is cancer, breast cancer, colorectal cancer, liver cancer, kidney cancer, brain cancer, pancreatic cancer, lung cancer, stomach cancer, uterine cancer, ovarian cancer, prostate cancer, testicular cancer, thyroid cancer, carcionoma, myeloma, sarcoma, leukemia, lymphoma, melanoma, hematological malignancy, arthritis, atherosclerosis, cardiovascular disease, cataracts, chronic obstructive pulmonary disease, hypertension, osteoporosis, periodontitis, diabetes, Alzheimer's disease, stroke, Parkinson's disease, multiple sclerosis, Crohn's disease, HIV, influzena, pneumonia, or MRSA. In some embodiments, the at least one patient cell comprises an immune cell, neutrophil, macrophage, natural killer cell, eosinophil, basophil, mast cell, dendritic cell, T cell or B cell or any combination thereof, and exposing the patient cell to the donor cell improves the immune activity of the patient cell. In some embodiments, the method includes administering G-CSF, filgrastim, lenograstim, or ancestim to the donor or patient. In some embodiments, the donor and/or patient is a mammal. In some embodiments, the donor and/or patient is a human.

Some embodiments pertain to the target cell made by the method recited above or elsewhere herein. Some embodiments pertain to a pharmaceutical composition comprising the target cell.

Some embodiments pertain to a kit for collecting blood from a patient. In some embodiments, the kit comprises liquid collection containers. In some embodiments, the liquid collection containers are configured to receive blood. In some embodiments, the liquid collection containers are blood collection tubes or vials. In some embodiments, the kit comprises a laboratory directive. In some embodiments, the kit comprises patient instructions.

In some embodiments, the kit comprises an enclosing container configured to house other components of the kit. In some embodiments, the kit comprises a shipping envelope configured to receive samples prepared using the kit. In some embodiments, the shipping envelope is prepaid. In some embodiments, the shipping envelope provides for overnight shipping.

In some embodiments, the kit comprises a national lab directive. In some embodiments, the lab directive provides instructions for testing the blood samples. In some embodiments, the kit comprises a lab requisition form. In some embodiments, the lab requisition form is for a national laboratory. In some embodiments, the lab requisition form is a Quest National Lab Requisition form. In some embodiments, the kit comprises a biohazard container. In some embodiments, the biohazard container is a bag. In some embodiments, the laboratory directive comprises blood drawing instructions.

In some embodiments, the kit comprises a patient self-evaluation form. In some embodiments, the self-evaluation form is a quality of life form. In some embodiments, the self-evaluation form is a SF-36 quality of life survey.

In some embodiments, the kit comprises a diagnostic testing unit. In some embodiments, the diagnostic testing unit comprises a diagnostic testing kit comprising one or more of a myeloid leukemia panel, a myeloid/lymphoid ratio assay, a lymphocyte proliferative response assay, a natural killer cytotoxicity assay, a T helper cell/killer T cell ratio assay, and/or a complete blood count assay. In some embodiments, the lymphocyte proliferative response assay is mitogen-based and/or antigen-based. In some embodiments, the diagnostic testing unit comprises biochemical and/or genetic biomarker assays. In some embodiments, the diagnostic testing unit comprises one or more of a senescence gene array, an aging gene array, and/or a senescence protein array. In some embodiments, the kit comprises a senescence gene array and/or aging gene array measure blood is configured to measure mononuclear cells. In some embodiments, the senescence protein array is configured to measure blood plasma proteins.

In some embodiments, the kit comprises instructions indicating that the diagnostic testing should be performed about every month. In some embodiments, the kit comprises instructions indicating that a physical examination of the patient should be performed about every 12 to 24 months. In some embodiments, the patient instructions and/or the self-evaluation form indicates that it should be completed about every three months. In some embodiments, the kit comprises instructions indicating that a baseline physical examination and diagnostic testing should be performed prior to treatment.

In some embodiments, the kit comprises a packing material. In some embodiments, the packing material is bubble wrap.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments and are not intended to be limiting in scope.

FIG. 1A depicts a schematic of an animal model being treated using an embodiment of a therapeutic treatments disclosed herein. FIG. 1B depicts a schematic of donor cells interacting with patient cells via a transwell plate to provide target cells.

FIG. 2A depicts instructions and tests that are provided some embodiments of a kit as disclosed herein. FIG. 2B depicts patient testing (which can be in the form of directives) information that is requested in or that is provide in some embodiments in the kit. FIG. 2C shows an embodiment of a kit and its contents. FIG. 2D shows another embodiment of a kit and its contents. FIG. 2E shows another embodiment of a kit and its contents. FIG. 2F-2J provide embodiments of laboratory directives and instructions that may be included in the kits.

FIG. 3A is a schematic overview of an embodiment of a clinical process for preparing therapeutic target cells for use in a human patient as disclosed in one or more of the following examples.

FIGS. 3B-3D provide alternative schematics of the clinical process, including detailing logistics for shipping and transport of cells to various facilities and patient locations.

FIG. 3E provides an overview of various facilities and locations. As shown, harvesting, storage, clinical, and testing facilities may be located in different areas (even in different countries). Based on these different locations, one or more of the disclosed shipping and or preservation techniques disclosed herein advantageously allow improved patient outcomes with increased cell vitality and viability.

FIG. 4 is a flow diagram showing an embodiment of a clinical process for preparing therapeutic target cells for use in a human patient.

FIG. 5 is a flow diagram depicting an embodiment of a clinical batch process for cell restoration.

FIG. 6 depicts an embodiment of the equilibration and seeding of aged and young cells in a transwell plate for treatment.

FIG. 7 depicts another embodiment of the equilibration and seeding of aged and young cells in a transwell plate for treatment.

FIG. 8 is a schematic depicting an embodiment of the clinical process after the transwell exposure step is performed. As shown, patients are infused with target cells at a clinical facility and are cell samples are tested at an external testing facility.

FIG. 9 is a diagram depicting shows an embodiment of the batch process for preparing target cells and treating a patient with the same.

FIG. 10 provides an additional embodiment of the cell restoration and/or treatment process, detailing the thawing of patient (B_{O} cells) and donor cells (Y05), the equilibration of such cells, the transwell restoration of the B_{O} cells to provide target cells, the transport of the target cells to the clinical facility, and the infusion of a patient (along with in process testing of cells).

FIG. 11 shows data regarding cell vitality using various cell media as base media.

FIG. 12 shows data regarding cell vitality using various cell media as base media.

FIG. 13 shows data from the clonogenic assay.

FIG. 14 shows cell vitality data in a number of incubation conditions.

FIG. 15 shows data regarding stem cell health of restored B_{O} cells 24 h post-restoration under various conditions.

FIG. 16 shows data pertaining to the recovery of restored B_{O} cells from time of transwell seeding.

FIG. 17 shows the evaluation of Young Donors' Ability to Restore Aged Donors in Xenogeneic-Free Restoration Media Stem Span (SS) supplemented with MEM Non-Essential Amino Acids Solution (NEAA), Insulin-Transferrin-Selenium (IST)-and H.S.A.

FIG. 18 provides data from the clonogenic assay confirms that maximal restoration of stem cell function is obtained when the young donor is utilized as a facilitator in Stem Span media supplemented with H.S.A., IST and NEAA and Sodium Pyruvate.

FIG. 19 provides an embodiment of a testing schedule after treatment.

FIG. 20 depicts a paired box 5 (PAX5) signaling cascade.

FIG. 21 depicts a chart displaying embodiments of strategies for treating a patient as disclosed herein.

FIGS. 22A-O depict alternative strategies for treating a patient using the methods disclosed herein. (A) and (B) depict an embodiment of an ex vivo treatment strategy. (C) depicts an embodiment of an in vivo treatment strategy.

FIGS. 22P1-22P30 depict the sequences for some embodiments of target genes, RNAi(s), and target proteins.

FIGS. 23A-E. Effect of young paracrine factors on aged hematopoietic and immune function. Total mononuclear cells (MNC) harvested from mobilized peripheral blood (MPB) of aged and young study donors, and umbilical cord blood (UCB) donors were measured for baseline levels of (A) CD45+ and (B) CD34+ cells, n=4. (C) Sustainability of aged cell restoration was measured by long-term culture-initiating cell (LTC-IC) assay. Aged cells from heterochronic and isochronic culture, and young controls, were harvested on days 3, 7, 10 and 15 of transwell culture and transferred to confluent monolayers of irradiated human stroma for long-term culture. At 6, 8 and 12 weeks after initial seeding, cells were harvested and evaluated by clonogenic assay for CFU-GM. (D) Baseline levels of HLA-DR was measured in aged, young and UCB donor samples, n=4. (E) To assess the immunogenic effects of heterochronic culture, the ability of restored aged cells to stimulate autologous naïve aged cells was measured by mixed lymphocyte reaction. Stimulation of naïve aged cells with young cells served as control, n=2. Results are presented as the mean±SEM. *p≤0.05 vs. control.

FIGS. 24A-J. Effect of young paracrine factors on aged hematopoietic and immune function. (A) Total mononuclear cells (MNC) harvested from mobilized peripheral blood (MPB) of aged and young study donors, and umbilical cord blood (UCB) donors were measured for baseline hematopoietic differentiation by clonogenic assay. Results are presented as mean number of colony forming units—granulocyte, monocyte (CFU-GM), n=4. MPB cells from aged and young donors were also measured for baseline (B) oxidative stress and (C) cell-mediated cytotoxicity. Oxidative stress was measured by mitosox assay, and delineated into mitosox negative, low and high populations by flow cytometry. Cytotoxicity was quantified by flow cytometry to determine % target lysis at multiple effector to target ratios, with donor MNC as effector and K562 as target. (D) For assessing aged cell restoration by young paracrine factors, MNCs were co-cultured using a 0.4 um transwell insert to separate the aged (bottom chamber) and young (top chamber) cells. At select time points up to day 15, aged cells from heterochronic (aged/young) or isochronic culture (aged/aged), or young cells from isochronic culture (young/young) were harvested and evaluated for functional restoration by clonogenic assay (E). Cells harvested from heterochronic and isochronic control cultures at day 7 were evaluated for (F) clonogenicity, (G) oxidative stress and (H) cell-mediated cytotoxicity. For clonogenic assessment, restoration was compared among young and UCB heterochronic cultures and isochronic controls after 7 days. (I) To evaluate whether in vitro restoration can be propagated, aged and young cells from isochronic cultures (+ iso aged, + young) or aged cells from heterochronic culture (+ hetero aged) were harvested at day 7 and transferred to fresh transwell cultures with naïve aged cells. As control, naïve aged cells were also placed in isochronic culture (gray bar). After an additional 7 days, aged cells from the 2nd set of cultures were evaluated by clonogenic assay. (J) Part of each study donor LeukoPak MPB was separated into CD34+ and CD34− fractions during initial cell processing. To determine a role for young CD34+ cells in the mechanism of restoration, 7-day heterochronic cultures were setup with either CD34-purified (Y34+) or CD34-depleted (Y34-) young cells, and restoration measured by clonogenic assay. Results are presented as the mean±SEM, n=3, unless otherwise noted. *p≤0.05 vs. control.

FIGS. 25A-G. Procedural and safety monitoring of huNSG mice. To determine the optimal dose for NSG engraftment, pilot study mice were transplanted with increasing doses of aged CD34+ cells and (A) initial engraftment, and (B) chimerism stability measured up to 20 weeks post-transplant. Human chimerism was determined by expression of huCD45 in blood. (C) Study design for huNSG aging cell restoration study. A total of 68 irradiated mice were transplanted with aged (n=56) or young (n=12) CD34+ cells, with 34 of 56 mice successfully engrafted with aged, and 12 of 12 mice successfully engrafted with young CD34+ cells. Chimerism cutoff for enrolling mice in the heterochronic and isochronic culture treatment arms was a minimum of 1% huCD45+ cells in blood. Mice displaying 0.5%-1% chimerism were enrolled in the saline treatment arms, and mice displaying <0.5% chimerism were not enrolled in the study at all. (D) Chimerism stability was monitored during the initial 19-week engraftment period and compared among individual aged donors (left plot) and among donor age group (middle plot). Chimerism stability after the 2nd transplant was monitored for 14 weeks up to study endpoint for all treatment groups (right plot). (E) Kaplan-Meier plot for huNSG overall survival (F) mouse body weights for the 14 weeks following the 2nd transplant are shown. Percent survival is displayed in the legend inset. (G) Mouse spleen weights at study endpoint, with spleen images in legend inset. Results are presented as the mean±SEM. *p≤0.05 vs. control.

FIGS. 26A-K. Creation of a humanized mouse model to evaluate restoration of the aging lymphohematopoietic system. (A) Humanized mouse study design. 6-week old female NSG mice were sub-lethally irradiated and transplanted with human aged or young CD34+ cells. After 19 weeks to allow for engraftment and sustained human hematopoiesis, mice were transplanted with autologous, CD3-depleted cells from 7-day heterochronic (Aged+Rest, n=12) or isochronic control cultures (Aged+Non-Rest, n=11; Young, n=8). 14 weeks after the second transplant, mice were sacrificed and tissues harvested. (B) Bone marrow (BM) and blood was measured for human leukocyte chimerism by expression of huCD45 in all treatment groups. Mice humanized with cells from aged donors A01 and A02, and young donor Y01, were given the nomenclature huNSG-donor ID. huCD45+ cells from blood were probed for (C) human CD3 and (D) human CD33, and from BM for (E) human CD34. To determine changes in immune phenotype metrics related to aging, the ratio of (F) CD4+ to CD8+ human leukocytes in blood, and (G) lymphoid (CD3++CD19+) to myeloid (CD33+) human leukocytes in BM and blood were determined. (H) BM was harvested and colony forming ability measured by clonogenic assay. Colony formation from BM of non-humanized mice served as background control. (I) MNC were isolated from blood and cultured ex vivo in the absence (unstimulated, left graph) or presence (stimulated, right graph) of CD3/CD28-conjugated beads. After 72 h, human leukocytes were measured for CD4+ and CD8+ T cell activation by expression of the activation marker CD25. (J) Blood plasma from huNSG receiving restored or non-restored cells was isolated and the expression of 68 known senescence-associated secretory factors (SASFs) measured by custom protein array. Semi-quantitative densitometry was utilized to perform expression analysis, with a 1.5-fold cutoff for classifying up- or down-regulation, or no change. (K) Engrafted human cells were purified from mouse BM, and RNA isolated for gene expression studies evaluating 145 genes related to human senescence and aging by qPCR. Results were normalized to housekeeping genes and differential expression determined, with a 1.5-fold cutoff for classifying up- or down-regulation, or no change. Differential gene and protein expressions are represented by heatmap in J and K. Results are presented as the mean±SEM, unless otherwise noted. *p≤0.05 vs. control.

FIGS. 27A-B. Histologic evaluation of tissues from huNSG treatment groups. At study endpoint, major organs and immune tissues were harvested. (A) H&E staining of mouse BM (top panels) and spleen (bottom panels), 10× magnification. (B) All harvested tissues were examined by a pathologist for tissue necrosis and tumorigenesis. Treatment groups were compared to age-matched control tissue for pathological comparison.

FIGS. 28A-F. Phenotypic characterization of human hematopoietic and immune cells in huNSG immune tissues. Blood, BM and spleen from all treatment groups were harvested and measured for human leukocyte (huCD45+) populations, including (A) hematopoietic stem (CD34+38−) and progenitor cells (CD34+38+); (B) T cells (CD3+); (C) T helper (CD4+) and cytotoxic (CD8+) cells; (D) natural killer cells (CD3−56+); (E) B cells (CD19+); and (F) myeloid cells (CD33+). Results are presented as the mean±SEM.

FIGS. 29A-C. Senescence- and aging-related gene and protein expression in huNSG treatment groups. (A) Scatterplots comparing senescence-associated secretory factor (SASF) expression in plasma of mice transplanted with either aged restored (left plot) or non-restored (right plot) cells compared to young. Values are normalized by background subtraction of SASF levels in non-humanized control NSG mice. Results are presented as mean densitometry units, with description of upregulated, downregulated and no change in expression SASFs listed in (B). (C) List of aging- and senescence-related genes whose expression is upregulated, downregulated or no change in human cells isolated from huNSG BM. Classifications in B and C are based on a 1.5-fold change cutoff.

FIGS. 30A-F. Characterizing exosomes and exosomal miRNAs in heterochronic and isochronic cultures. (A) Nanoparticle tracking analysis (NTA) of exosomes isolated from 7-day heterochronic and isochronic cultures at day 3 and day 7. Effect of the AGO2 inhibitor, BCI-137, on exosome (B) production, (C) total (left panel), small (right panel) and (D) micro RNA content in heterochronic culture. Enrichment of exosomal miRNAs in cultures without inhibitor vs. with inhibitor is depicted by scatterplot in D with a 1.5-fold change cutoff. (E) Ingenuity Pathway Analysis (IPA) of commonly expressed miRNAs that are differentially expressed (1.5-fold cutoff) in exosomes of young vs. aged isochronic cultures. (F) Validation of miFinder qPCR array by individual qPCR experiments in array (left panels) and fresh donor samples (right panels). Gating scheme depicts miRNAs that are upregulated in young isochronic and heterochronic vs. aged isochronic cultures. Results are depicted by scatterplot with 1.25-fold and 1.05-fold cutoffs in array and fresh donor samples, respectively. Array and individual qPCR studies were normalized to RNU6, SNORD68 and SNORD95 and presented as fold change, with a value of 1 representing control.

FIGS. 31A-G. Ascribing a role for exosomes in the mechanism of cellular restoration. (A) Exosomes were isolated from 7-day heterochronic and isochronic control cultures on day 4 and day 7, and then pooled and quantified by nanoparticle tracking analysis (NTA), n=4. (B) Pooled exosomes from A were added to fresh aged isochronic cultures (middle bars) at a dose of 1×106 exosomes/culture on day 0 and again on day 4 of 7-day culture. Non-supplemented heterochronic and isochronic cultures served as control. After 7 days, cells were harvested and measured for CFU-GM by clonogenic assay. (C) Total RNA was extracted from exosomes from A and quantified to determine total RNA content/exosome, n=8. (D) The AGO2 inhibitor, BCI-137, was added to heterochronic cultures upon initial seeding (far right bar) and effect on clonogenicity was established compared to no inhibitor and control isochronic cultures. (E) 3D plots comparing 84 commonly expressed miRNAs among exosomes harvested from isochronic (left panel) and heterochronic cultures (middle and right panels) by qPCR. Results were normalized to housekeeping genes within the array, and are presented as fold difference, with a value of 1 representing no change. (F) Venn diagrams illustrate expression of 68 of 84 commonly expressed miRNAs in exosomes isolated from all cultures. Overlapping areas represent miRNAs with less than 1.5-fold difference among groups. 25 of 68 expressed miRNA show no change among all groups. (G) Among exosomal miRNAs that were differentially expressed, only miR-19a, miR-103a, miR-106b and miR-146a were consistently upregulated in both young isochronic cultures and heterochronic cultures. Results are presented as the mean±SEM, n=3, unless otherwise noted. Array and individual qPCR studies were normalized to RNU6, SNORD68 and SNORD95 and presented as fold change, with a value of 1 representing control. *p≤0.05 vs. control.

FIGS. 32A-H. Characterizing the young intracellular miRnome and ascribing a role for miRNAs in the mechanism of restoration. (A) Small RNA was purified from aged, young and UCB isochronic cultures for whole miRnome sequencing. All miRNAs exhibiting greater than 100 mappable reads were further analyzed. Differential RNA expression is denoted by heatmap, with miRNA exhibiting greater than 1.4-fold difference among aged vs. UCB and young samples tabulated. Outer area of the Venn diagrams depicts total number of intracellular miRNAs with greater than 100 mappable reads in age-matched isochronic samples. Overlapping areas represent common miRNA among samples. (B, C) Similar studies as in A were carried out comparing miRNAs sequenced from aged isochronic and heterochronic (young-aged, UCB-aged) samples. (D) miRNA showing differential expression from A were compared to miRNA showing increased or decreased expression in heterochronic (aged-young) vs. aged isochronic cultures in B and C, and results tabulated to illustrate candidate miRNAs whose expression patterns are coincident with aged cell restoration. (E) Scatterplot depicting linear correlation between exosomal miR-7641-2 expression and total mappable reads, n=10. (G) Expression of early exosomal candidate miRNAs from miFinder array studies in sequencing dataset. Results are shown for isochronic and heterochronic cultures as average reads per 10000 total mapped reads. (H) To evaluate whether candidate exosomal miRNAs can be propagated after aged cell restoration, aged and young cells from 7-day isochronic cultures or aged cells from heterochronic culture were harvested at day 7 and transferred to fresh transwell cultures with naïve aged cells for an additional 7 days. On the 3rd (day 10) and 7th (day 14) day of the propagation culture, exosomes were isolated and probed for candidate miRNA expression by qPCR. Results were normalized to miR-7641-2 expression and presented as fold change, with a value of 1 representing control. Results are presented as the mean±SEM, n=3, unless otherwise noted. *p≤0.05 vs. control.

FIGS. 33A-K. Characterizing the young exosomal miRnome and ascribing a role for miRNAs in the mechanism of restoration. (A) Exosomes were isolated from aged, young or UCB isochronic cultures and small RNA purified for whole miRnome sequencing. All miRNAs exhibiting greater than 100 mappable reads were further analyzed. Differential RNA expression is denoted by heatmap, with miRNA exhibiting greater than 1.4-fold difference among aged and young samples tabulated. Outer area of the Venn diagrams depicts total number of exosomal miRNA with greater than 100 mappable reads in age-matched isochronic samples. Overlapping areas represent common miRNA among samples. (B, C) Similar studies as in A were carried out comparing miRNAs sequenced from exosomes of aged isochronic and heterochronic (young-aged, UCB-aged) samples. (D) miRNA showing differential expression from A were compared to miRNA showing increased expression in heterochronic (aged-young) vs. aged isochronic cultures in B and C, and results tabulated to illustrate candidate miRNAs whose expression patterns are coincident with aged cell restoration. (E) Exosomes collected from heterochronic and isochronic cultures of different study donors were used to validate expression of candidate miRNA by individual qPCR. Results were normalized to miR-7641-2 and presented as fold change, with a value of 1 representing aged control. (F) 6 of 8 miRNA passing qPCR validation were tested for their ectopic ability to restore aged cell function by clonogenic assay. Candidate miRNA were tested alone (left panel) or in various combinations (right panel) vs. negative control RNA and non-transfected control (NTC). (G) miRNA formulations demonstrating significant improvement in aged clonogenicity were further evaluated for ability to enhance CD4+ (top panels) and CD8+ (bottom panels) T cell activation in aged donors. Cells were stimulated with anti-CD3 and -CD28 (right panels), or unstimulated (left panels), and T cell activation measured after 72 h by expression of the activation marker CD25. (H) Formulations demonstrating a significant effect on T cell activation in G were finally evaluated for ability to boost cell-mediated cytotoxicity compared to control RNA. To investigate whether miRNA candidates also have a role in young cell function, cells from young study donors were transfected with candidate anti-miRNAs, either alone (anti-619) or in combination (anti-619, -1303 and -4497), and effect on (I) clonogenicity, (J) T cell activation and (K) cell-mediated cytotoxicity as in F, G and H, respectively, were determined compared to anti-miR control RNA. Results are presented as the mean±SEM, n=3, unless otherwise noted. *p≤0.05 vs. control.

FIGS. 34A-G. Identification of potential young exosomal miRNA targets in aged cells. (A) Up- and downregulated intracellular miRNAs comparing aged heterochronic (aged young) vs. isochronic cultures with a 1.5-fold cutoff, and their (B) predicted activation/inhibition networks after IPA. Up- and downregulated (C) exosomal miRNAs comparing UCB vs. aged isochronic and (D) intracellular miRNAs comparing aged heterochronic (aged-UCB) vs. isochronic cultures with a 1.5-fold cutoff. (E) Illustration of the top cellular functions (left graph) and canonical pathways (right graph) predicted by (F) these networks are shown. (G) Validation of siRNA knockdown of target candidates in cells from aged donors. Results were normalized to β-Actin expression and presented as fold change, with a value of 1 representing control (scrambled siRNA). Results are presented as the mean±SEM, n=3, unless otherwise noted. *p≤0.05 vs. control.

FIGS. 35A-L. Identification of exosomal miRNA targets that promote restoration of aged cells. (A) miRNA that were differentially expressed in young exosomes compared to aged, and intracellularly in aged hetrochronic vs. isochronic cultures, were analyzed by Ingenuity Pathway Analysis (IPA). Illustration of the top cellular functions (left graph) and canonical pathways (right graph) predicted by these networks are shown. (B) Radial depiction of the young exosomal-aged heterochronic intracellular interactome. p53 is at the center of the overlapping network predictions. Direct interactions among the networks are displayed. (C) The 6 qPCR-validated miRNAs from the sequencing studies were probed for potential targets using the TargetScan human database. A total of 6101 potential targets were evaluated, with number of common targets within the group of 6 miRNA displayed within the descending concentric circles. 25 targets were identified that met the conditions, either: (1) ≥4 common hits among the miRNA group, including miR-619 OR miR-1303; or (2) ≤3 common hits among the group, including miR-619 AND miR-1303. Predicted expression of these targets was analyzed by IPA and (D) the resulting network predictions compared to the young exosomal and aged heterochronic intracellular miRNA interactome. (E) Targets satisfying the above miRNA hit conditions were tabulated and pared down based on expression in relevant tissues (target gene encodes verified protein; expression not limited solely to neural tissue) and predicted interaction with the miRNA interactome to (F) yield 5 potential downstream targets for functional validation. (G) RNA collected from aged cells in heterochronic or isochronic culture (top plot), or human cells purified from BM of huNSG transplanted with restored or non-restored cells (bottom plot), was probed for expression of candidate targets by qPCR. Results were normalized to j-Actin and presented as fold change. (H) Basal expression of PAX5 and PPM1F in aged donor cells was determined by qPCR, with results presented as fold change versus young donor expression, which were arbitrarily assigned a value of 1. (I) Aged donor cells were transfected with candidate pre-miRs or control RNA (first and second groups of bars from left) and young donors were transfected with candidate anti-miRs or control RNA (third and fourth group of bars from left), and effect on expression of PAX5 and PPM1F was determined by qPCR, with results presented as fold change versus control RNA, which were arbitrarily assigned a value of 1. (J) Effect of siRNA knockdown of PAX5 or PPM1F on T cell activation was performed for CD4+(top panels) and CD8+(bottom panels) populations. Percent activated vs. total T cells is presented (right panels) for each condition. (K) Aged cells were transfected with siRNA to target candidates, PAX5 or PPM1F, or scrambled siRNA control, and effect on clonogenicity measured compared to heterochronic and isochronic controls. (L) Target knockdown cells were finally evaluated for ability to boost cell-mediated cytotoxicity compared to control RNA. Results are presented as the mean±SEM, n=3, unless otherwise noted. *p≤0.05 vs. control.

FIGS. 36A-L. Application of the humanized model of the aging lymphohematopoietic system to test cell-free methods of restoring aged function. (A) Humanized mice were created using 2 different aged (A03, A04) and young donors (Y03, Y04), with the exception that 15 weeks was allowed for engraftment and sustained human hematopoiesis. Aged CD34+-engrafted mice were then transplanted with autologous, CD3-depleted cells that had been transfected for 7 days with either miR-619 alone (n=18), a miR-combo of -619, -1303 and -4497 (n=18) or control RNA (n=18). 15 weeks after the second transplant, mice were sacrificed, and tissues harvested. (B) Bone marrow (BM) and blood was measured for human leukocyte chimerism by expression of huCD45 in all treatment groups. huCD45+ cells from blood were probed for (C) human T cell populations in blood (from left to right; CD3, CD4, CD8 and CD4/CD8 ratio). Data from all mice transfected with either miR formulation (619 alone or in combination) were pooled and compared to negative control for (D) human B cell populations (CD19) in BM and blood, and (E) pan myeloid cells (CD33) and (F) lymphoid to myeloid ratio (CD3++CD19+/CD33+) in BM. (G) BM was harvested and colony forming ability measured by clonogenic assay. Colony formation from BM of non-humanized mice served as background control. (H) MNC were isolated from blood and cultured ex vivo in the absence (unstimulated) or presence (stimulated) of CD3/CD28-conjugated beads. After 72 h, human leukocytes were measured for CD4+ and CD8+ T cell activation by expression of the activation marker CD25. (I) Engrafted human cells were purified from mouse BM, and RNA isolated to examine gene expression of target candidates, PAX5 (left bars) and PPM1F (right bars), by qPCR. (J) Isolated RNA was also probed for microarray studies evaluating 145 genes related to human senescence and aging by qPCR. Results were normalized to housekeeping genes and differential expression determined, with a 1.5-fold cutoff for classifying up- or down-regulation, or no change. (K) Blood plasma was isolated and the expression of 68 known senescence-associated secretory factors (SASFs) measured by custom protein array. Semi-quantitative densitometry was utilized to perform expression analysis, with a 1.5-fold cutoff for classifying up- or down-regulation, or no change. Differential gene (J) and protein expressions (K) are represented by heatmap (left panel), pie charts (top panels) and bar graphs (bottom panels). (L) Conditioned cell culture media from H were probed for cytokine expression using a human T-cell cytokine array. Semi-quantitative densitometry was utilized to perform expression analysis, with non-conditioned culture media used as control for background subtraction. Differential cytokine expression is represented by heatmap (left panel) and bar graphs (right panels) which normalized to unstimulated control. Results are presented as the mean±SEM, unless otherwise noted. *p≤0.05 vs. control.

FIGS. 37A-G. Procedural and safety monitoring of humanized mice from the cell-free restoration study. (A) Study design for the cell free restoration study in humanized mice. A total of 170 irradiated mice were transplanted with aged (n=120) or young (n=50) CD34+ cells, with 54 of 120 mice successfully engrafted with aged, and 30 of 50 mice successfully engrafted with young CD34+ cells. Chimerism cutoff for enrolling mice in the control and treatment arms was a minimum of 1% huCD45+ cells in blood. Mice displaying 0.5%-1% chimerism were enrolled in the saline treatment arms (not shown), and mice displaying <0.5% chimerism were not enrolled in the study at all. (B) Bleeds were performed on mice transplanted with aged (A03 or A04) and young donor (Y03 and Y04) CD34+ cells, and chimerism evaluated at 9- and 15-weeks post-transplant. Average chimerism of the aged (top graph) and young (bottom graph) donors enrolled in the study are shown. (C) Kaplan-Meier plot for huNSG overall survival post-treatment and (D) mouse body weights for the 15 weeks following the 2nd transplant are shown. Percent survival is displayed in the legend inset. (E) Mouse spleen weights at study endpoint, with spleen images in legend inset. Total (F) spleen and (G) bone marrow cellularity at study endpoint are displayed. Results are presented as the mean±SEM.

FIGS. 38A-B. Histologic evaluation of tissues from huNSG treatment groups in expanded study. (A) At study endpoint, major organs and immune tissues were harvested. (A) H&E staining of mouse spleen (top panels) and bone marrow (bottom panels), 4× magnification. (B) All harvested tissues were examined by a pathologist for tissue necrosis and tumorigenesis. Treatment groups were compared to age-matched control tissue for pathological comparison.

FIGS. 39A-C. Senescence- and aging-related gene and protein expression in huNSG treatment groups from expanded study. (A) Scatterplots comparing senescence-associated secretory factor (SASF) expression in plasma of mice transplanted with either aged+negative control, aged+miR-619 or aged+miR-combo cells compared to young control. Values are normalized by background subtraction of SASF levels in non-humanized control NSG mice. Results are presented as mean densitometry units, with average total SASF expression among each group also shown for comparison (far right bar graph). Enumeration of SASFs upregulated, downregulated or not changed for miR-619 vs. control (left table) or miR-combo vs. control (right table) is listed in (B). (C) List of aging- and senescence-related genes whose expression is upregulated, downregulated or not changed in human cells isolated from huNSG BM in miR-combo treated mice vs. control. Classifications in B and C are based on a 1.5-fold change cutoff. Results are presented as the mean±SEM. *p≤0.05 vs. control.

FIGS. 40A-F. Comparative analysis of heterochronic- vs. miR-treated aged humanized mouse studies. To indirectly compare heterochronic- vs. miRNA-mediated cell restoration in the humanized mouse studies, data were normalized to non-restored isochronic control and negative RNA control mice, respectively. Results are presented as the mean±SEM with control values set to either 1 or 0. *p≤0.05 vs. control. Comparative data were presented for (A) human T cell populations in blood (from left to right; CD3, CD4, CD8 and CD4/CD8 ratio); (B) human B cell populations (CD19) in blood; (C) pan myeloid cells (CD33) and lymphoid to myeloid ratio (CD3++CD19+/CD33+) in BM; (D) colony forming ability measured by clonogenic assay; (E) aging (left graph) and senescence (right graph) arrays; and (F) SASP protein array.

FIGS. 41A-C. Cartoon depicting mechanism of cellular restoration. (A) Heterochronic transwell culture displaying young (top chamber) and aged (bottom chamber) cells in co-culture separated by a membrane containing 0.4 m pores. Exosomes released by young cells penetrate the transwell pores and perfuse the aged cells in the bottom chamber. (B) The young exosomes fuse with the aged cell membranes to deliver their payload of RNA, DNA, lipids and proteins intracellularly to the aged cells. (C) Specific miRNAs that are elevated in the young cells, but not expressed basally by the aged cells (miR-619, -1303 and -4497), are delivered by the young exosomes into the aged cytoplasm where they selectively bind target mRNAs (PAX5, PPM1F) for translational repression. Both PAX5 and PPM1F have downstream targets that are involved in cellular senescence, including p53 and p21.

FIG. 42. Cartoon depicting clinical implementation of restoration technology. Two adoptive, autologous immune restoration therapies have been modeled in humanized mice, which could be translated to human studies. Studies would utilize aged, healthy individuals >59 y/o who had previously undergone stem cell mobilization and banking. Patients would be administered an autologous cell therapy utilizing either young cells (left) or an off-the-shelf biologic (right) as the restorative agent. Patients' immune function would be tested before and after treatment to assess safety and efficacy using a number of biomarker-based assays and patient reported outcomes.

FIGS. 43A-B show viability and cell number data for Example 3, respectively.

FIG. 44 is a depiction of an embodiment for immunophenotyping a cell sample.

FIG. 45 is a depiction of an embodiment of a transwell co-culture experimental apparatus.

FIG. 46 is a plot of a gene expression analysis for donor cell samples and receiver cell samples.

FIG. 47 is a plot of a protein expression analysis for donor cell samples and receiver cell samples.

FIGS. 48A and B are a plot of a level of expression of the indicated proteins for the donor cell samples and receiver cell samples.

FIG. 49 is a plot of the average telomere length for the donor cell samples and receiver cell samples.

FIG. 50 is a plot of a gene expression analysis for baseline donor cell samples and restored cell samples.

FIG. 51 is a plot of a protein expression analysis for baseline donor cell samples and restored cell samples.

FIG. 52A is a plot of a protein expression analysis for baseline donor cell samples and baseline receiver cell samples. FIG. 52B is a plot of a protein expression analysis for baseline donor cell samples and baseline donor cell samples and restored cell samples.

FIG. 53A is a plot of a protein expression analysis for the baseline donor cell sample and the baseline receiver cell sample R1. FIG. 53B is a plot of a protein expression analysis for the baseline donor cell sample and restored cell sample R1-D1. FIG. 53C is a plot of a protein expression analysis for the baseline donor cell sample and restored cell sample R1-D2.

FIG. 53D is a plot of a protein expression analysis for the baseline donor cell sample and restored cell sample R1-D3.

FIG. 54 is a plot of a level of protein expression in restored cells in the presence or absence of manumycin.

FIG. 55 is a plot of the telomere length for the restored cell sample from the donor cell sample-receiver cell sample pair R1-D1 in the presence or absence of manumycin.

FIGS. 56 and 57 depict the results of the natural killer cell assay for the samples from Example 8.

FIG. 58 depicts the results of the clonogenic assay for the samples from Example 8.

FIGS. 59A-D depict the results of a flow cytometry assay for the samples from Example 8.

FIG. 60 depicts the results of a propagation of restoration experiment for the samples from Example 9.

FIG. 61A depicts myeloid to lymphoid ratios of patients treated with AR-100.

FIG. 61B depicts neutrophil to lymphocyte ratios of patients treated with AR-100.

FIG. 62A depicts the relative immune response in patients treated with AR-100 following mitogen stimulation. FIG. 62B depicts the relative immune response in pateints treated with AR-100 following antigen stimulation.

FIG. 63 depicts the cytotoxic response of natural killer cells of patients treated with AR-100.

FIG. 64 depicts the effect of AR-100 on the expression of aging- and senescence-related genes in patients.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Some embodiments disclosed herein pertain to methods of preparing target cells and treating patients with the same. In some embodiments, one or more process steps disclosed herein for the preparation of target cells provide surprisingly increased viability and/or yield of the target cells. In some embodiments, by preparing cells using one or more techniques disclosed herein, improved patient outcomes (including increased vitality of target cells, etc.) can be achieved. In some embodiments, because diagnostic facilities, clinical facilities, cell laboratories, biorepositories, and processing laboratories may be in different locations (including at different facilities in different states or even in different countries), one or more of the disclosed methods can be used to improve testing, storage, and treatment outcomes. In some embodiments, disclosed herein are transport methods for patient, donor, and target cells that achieve increased viability and quality of such cells. Some embodiments disclosed herein pertain further to methods of using target cells for treating patients in need of treatment. In some embodiments, the methods of treatment include an administration of target cells or materials isolated to a patient suffering from, for example, an age-related disease, cancer, an infectious disease, or the like.

Some embodiments disclosed herein pertain provide kits for use in providing treatment and cellular restoration. Some embodiments disclosed herein pertain to methods of preparing target cells and treating patients with the same. In some embodiments, one or more process steps disclosed herein for the preparation of target cells provide surprisingly increased viability and/or yield of the target cells. In some embodiments, by preparing cells using one or more techniques kits as disclosed herein, improved patient outcomes (including increased vitality of target cells, etc.) can be achieved. In some embodiments, because diagnostic facilities, clinical facilities, cell laboratories, biorepositories, provide certain tests and processing laboratories may be in different locations (including at different facilities in different states or even in different countries), one or more of the disclosed methods can be used information to improve testing, storage, and treatment outcomes. In some embodiments, disclosed herein are transport methods for patient, donor, and target cells that achieve increased viability and quality of such cells. Some embodiments disclosed herein pertain further to methods of using target cells for treating patients in need of seeking such treatment. In some embodiments, the disclosed kits provide particular testing panels that are useful in determining whether a patient can be so treated. In some embodiments, the kits provide particular reagents for mobilizing patient cells for treatment. In some embodiments, the methods of treatment kits provide questionnaires and evaluation tools to provide monitoring of patient progress after treatment with one or more target cells. In some embodiments, a kit may include an administration of target cells or materials isolated to a patient suffering from, one or more therapeutic agents and/or compositions for example, an preventing or treating age-related disease, cancer, an infectious disease, dysfunction and/or dysfunction that is not related to aging but that manifests biological and physiological outcomes that are similar or the like same as those found in aging cells.

The following description provides context and examples, but should not be interpreted to limit the scope of the inventions covered by the claims that follow in this specification or in any other application that claims priority to this specification. No single component or collection of components is essential or indispensable. Any feature, structure, component, material, step, or method that is described and/or illustrated in any embodiment in this specification can be used with or instead of any feature, structure, component, material, step, or method that is described and/or illustrated in any other embodiment in this specification.

Unless otherwise defined, all technical and scientific terms used herein have the same meaning as commonly understood by one of ordinary skill in the art to which this subject matter belongs. The terminology used in the description of the subject matter herein is for the purpose of describing particular embodiments only and is not intended to be limiting of the subject matter.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The “individual”, “patient” or “subject” treated as disclosed herein is, in some embodiments, a human patient, although it is to be understood that the principles of the presently disclosed subject matter indicate that the presently disclosed subject matter is effective with respect to all vertebrate species, including mammals, which are intended to be included in the terms “subject” and “patient.” Suitable subjects are generally mammalian subjects. The subject matter described herein finds use in research as well as veterinary and medical applications. The term “mammal” as used herein includes, but is not limited to, humans, non-human animals, including primates, cattle, sheep, goats, pigs, horses, cats, dogs, rabbits, rodents (e.g., rats or mice), monkeys, etc. Human subjects and patients include neonates, infants, juveniles, adults and geriatric subjects. The subject can be a subject “in need of” the methods disclosed herein can be a subject that is experiencing a disease state and/or is anticipated to experience a disease state, and the methods and compositions of the invention are used for therapeutic and/or prophylactic treatment. A subject can be a patient, which refers to a human presenting to a medical provider for diagnosis or treatment of a disease. A subject can be afflicted with or is susceptible to a disease or disorder but may or may not display symptoms of the disease or disorder. In some embodiments disclosed herein, an aged patient or subject can be one having an age that is greater than or equal to about: 40, 50, 60, 70, 80, 90, or ranges including and/or spanning the aforementioned values. In some embodiments disclosed herein, a young subject or patient can be one having an age that is less than or equal to 39, 30, 20, 10, or ranges including and/or spanning the aforementioned values.

The term “effective amount,” as used herein, refers to that amount of a recited compound that imparts a modulating effect, which, for example, can be a beneficial or desirable effect (biological or clinical), to a subject afflicted with a disorder, disease or illness (or at risk of developing the same), including improvement in the condition of the subject (e.g., in one or more symptoms), delay or reduction in the progression of the condition, prevention or delay of the onset of the disorder, and/or change in clinical parameters, disease or illness, etc. For example, an effective amount can refer to the amount of a composition, compound, or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In some embodiments, an improvement in a condition can be a reduction in age-related disease. In some embodiments, an improvement can be increase immune response in a subject. Actual dosage levels of active ingredients in an active composition of the presently disclosed subject matter can be varied so as to administer an amount of the active compound(s) that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein. In some embodiments, a “therapeutically effective amount” means a sufficient amount of the compositions disclosed herein to treat, prevent, and/or ameliorate one or more symptoms of the medical condition. It also may include a safe and tolerable amount of the compositions and/or agents disclosed herein, as based on industry and/or regulatory standards.

In some embodiments, the effectiveness of the compound or composition (including a cellular composition) is measured by the decrease in expression level of the protein of interest. For example, an effective decrease in expression can be at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

“Treat” or “treating” or “treatment” refers to any type of action that imparts a modulating effect, which, for example, can be a beneficial effect, to a subject afflicted with a disorder, disease or illness, including improvement in the condition of the subject (e.g., in one or more symptoms), delay or reduction in the progression of the condition, and/or change in clinical parameters, disease or illness, curing the illness, etc. As used herein, these can refer to a clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. “Treatments” refer to one or both of therapeutic treatment and prophylactic or preventative measures. Those in need of treatment include those already affected by a disease or disorder or undesired physiological condition as well as those in which the disease or disorder or undesired physiological condition is to be prevented. In some embodiments, the subject is administered the compositions disclosed herein in a therapeutically effective amount sufficient for treating, preventing, and/or ameliorating one or more symptoms of a medical condition, disorder, disease, or dysfunction. Hereinafter, for simplicity, the unwanted condition which has been used interchangeably with the terms medical condition, disorder, disease, and dysfunction are collectively referred to as the “medical condition.” As used herein, amelioration of the symptoms of the medical condition by administration of a particular composition of the type disclosed herein refers to any lessening, whether lasting or transient, which can be attributed to or associated with administration of compositions of the type disclosed herein. The term “treat” can also be used to denote a decrease in expression level of the protein in question.

As used herein, “pharmaceutically acceptable” refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein is intended to include any and all solvents, dispersion media, coatings, antibacterial and antifungal agents, isotonic and absorption delaying agents, and the like, compatible with administration to humans or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals. The term diluent, excipient, and/or “carrier” can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to or at least about 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism.

Herein a “mobilizer” or a “mobilizer of hematopoietic stem cells or progenitor cells” (used interchangeably) refers to any substance, whether it is a small organic molecule, synthetic or naturally derived, or a polypeptide, such as a growth factor or colony-stimulating factor or an active fragment or mimic thereof, a nucleic acid, a carbohydrate, an antibody, or any other agent that acts to enhance the migration of stem cells from the bone marrow into the peripheral blood. Such a “mobilizer” may increase the number of stem cells (e.g., hematopoietic stem cells or hematopoietic progenitor/precursor cells) in the peripheral blood, thus allowing for a more accessible source of stem cells for use in the methods disclosed herein. Any mobilizer suitable for increasing the number of stem cells in the subject that are available to be harvested and is compatible with the other aspects of this disclosure may be utilized. In an embodiment, the mobilizer is a cytokine such as granulocyte colony-stimulating factor (G-CSF). A commercial example of a mobilizer suitable for use in the present disclosure is NEUPOGEN® (filgrastim) which is a prescription medication used to treat neutropenia that is commercially available from Amgen. Another example of a mobilizer suitable for use in the present disclosure is a recombinant methionyl human stem cell factor which is commercially available as STEMGEN® from Amgen. Yet another example of a mobilizer suitable for use in the present disclosure is plerixafor which is an inhibitor of the CXCR4 chemokine receptor and blocks binding of its cognate ligand, stromal cell-derived factor-1α (SCF-1α) and is commercially available as MOZOBIL® from Genzyme.

As used herein, “exosomes” refers to small membrane vesicles released by cells, which contain a subset of proteins, lipids, and nucleic acids derived from the parent cell. In some embodiments, exosomes deliver nucleotides to cells. In some embodiments, exosomes are produced naturally by cells. In other embodiments, synthetic exosomes that are not produced naturally can be used to deliver nucleotides to cells.

As used herein, “in vivo” is given its ordinary meaning and refers to the performance of a method inside living organisms, usually animals, mammals, including humans, and plants, as opposed to a tissue extract or dead organism.

As used herein, “ex vivo” is given its ordinary meaning and refers to the performance of a method outside a living organism with little alteration of natural conditions.

As used herein, “in vitro” is given its ordinary meaning and refers to the performance of a method outside of biological conditions, e.g., in a petri dish or test tube.

As used herein, “quality” of cells may refer to one or more properties of cells at any stage after collection, including whether they form clumps, whether they are aseptic, whether the cells are useable for administration, and the like.

### Introduction

Aging is a biological process and the leading risk factor for the chronic diseases that account for the bulk of morbidity, mortality and health costs. The complexity of organismal aging appears to be driven by cellular dysfunction at the macromolecular and/or organelle level, which ultimately leads to a decline in tissue function and the manifestation of disease. As cells age they undergo epigenetic alterations that lead to dynamic changes in gene expression and increased likelihood of oncogenesis and cellular transformation. A potent inducer of cellular senescence is epigenomic stress, which can result from direct DNA damage, dysfunctional telomeres, disrupted chromatin, or strong mitogenic signals. Epigenetic alterations are biochemical modifications of DNA or DNA-associated proteins such as histones, which result in chromatin remodeling and functionally relevant changes to the genome, independent of altering the DNA sequence. One consequence of mounting epigenetic alteration is the increased likelihood of an oncogenic event that ultimately leads to cellular transformation and cancer development. Cells have a default protective mechanism to avert transformation through dramatic silencing of active chromatin. Once activated, this program leads to formation of heterochromatic foci and the entry of cells into a non-proliferative, metabolically active state of senescence.

Cell entry into the non-proliferative, yet metabolically active, state of senescence serves a protective role to avert transformation to an aberrant physiological form. However, senescent cells exhibit a profile of enhanced secretory factor production, termed the senescent-associated secretory phenotype (SASP). Many of the SASP factors are pro-inflammatory and/or tumor-supportive, thus cellular senescence is a fundamental aging mechanism tied to the progressive breakdown of tissue function with age. In particular, reduced function associated with an aging lymphohematopoietic system leads to compensatory increases in immune-related diseases, such as cancer. This decline in the lymphohematopoietic system and decreased immune surveillance is an important factor in the increased incidence of cancer, infectious diseases and immune-related disorders responsible for the majority of morbidity and health care expenditures in developed nations.

Epigenomic stress from sources other than age and/or other disease processes and disorders not associated with age can also lead to dysfunction that puts cells in a similar biological states to aged cells (e.g., pro-inflammatory and/or tumor-supportive status). These dysfunctions manifest in biological and physiological states that are similar to those found in aged cells.

There exists a need for methods and treatments that can intervene in the progressive breakdown of tissue function and may repair or stimulate aging or dysfunctional cells and tissues.

### Embodiments of Clinical Processing of Cells

Some embodiments disclosed herein pertain to compositions and methods for improving and/or restoring one or more cellular functions in cells (e.g., aged cells and/or cells from a patient in need of treatment). In some embodiments, the cellular functions may be directly or indirectly associated with promoting cellular health in a subject. In some embodiments, cellular function is improved in a target cell (or target cells). In some embodiments, the target cell can be introduced to a patient to achieve one or more beneficial effects in the patient. In some embodiments, disclosed herein are protocols that improve the quantity and quality of target cells. In some embodiments, the methods disclosed herein are viable for clinical use and include methods of transporting and preserving cells for use in cellular restoration. In some embodiments, these protocols include methods of isolating patient and/or donor cells, methods of preparing target cells using patient and/or donor cells, methods of transporting cells, methods of storing cells, and the like. In some embodiments, the processes for cellular manipulation achieve one or more of the following advantages or others: the process may be performed in an autologous and/or in an allogeneic manner; the co-culture-based restoration requires no genetic manipulation of cells; the procedure can utilize freshly collected cells (e.g., target cells are prepared and injected into patients in a matter of days, e.g., a period of days that is less than or equal to 5, 6, 7, 8, 10, 15, 30, or ranges including and/or spanning the aforementioned values) or cells that have been stored for long periods (e.g., patient, donor, or target cells that have been stored for a period of time that is greater than or equal to: 1 month, 6 months, 1 year, 2 years, 3 years, 5 years, 10 years, 15 years, 25 years, or ranges including and/or spanning the aforementioned values); from cell collection to administration, the process can be completed in a period of time that is less than or equal to: 1 week, two weeks, 3 weeks, one month, or ranges including and/or spanning the aforementioned values; the viability of cells collected and/or quantity of cells collected using one or more processes disclosed herein is improved by greater than or equal to about: 20%, 40%, 50%, 75%, 100%, or ranges including and/or spanning the aforementioned values; the quality of cells collected (as measured by one or more properties such as metabolic activity, increased expression of beneficial genes, decreased expression of undesired genes, etc.) using one or more processes disclosed herein is improved by greater than or equal to about: 20%, 40%, 50%, 75%, 100%, or ranges including and/or spanning the aforementioned values.

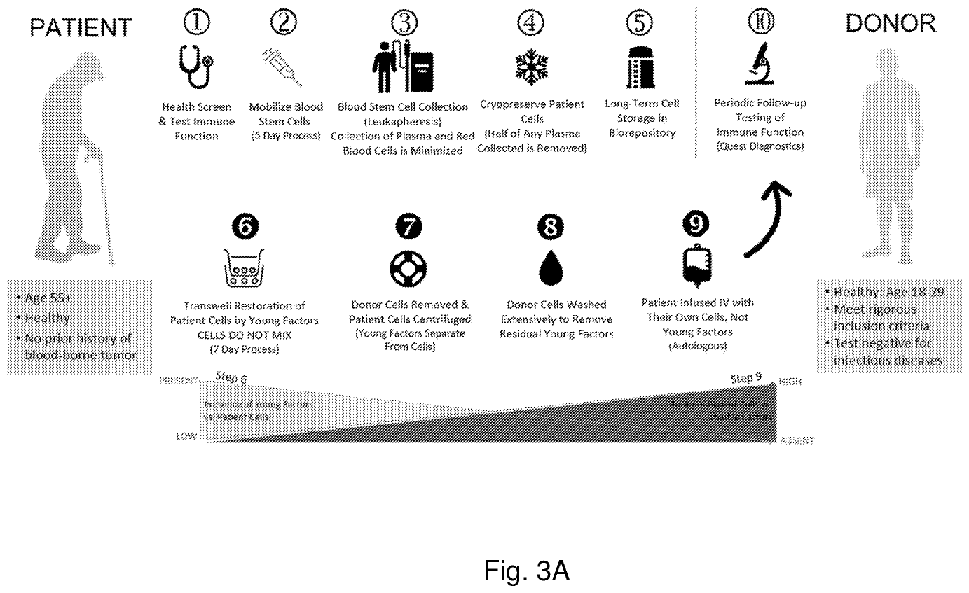

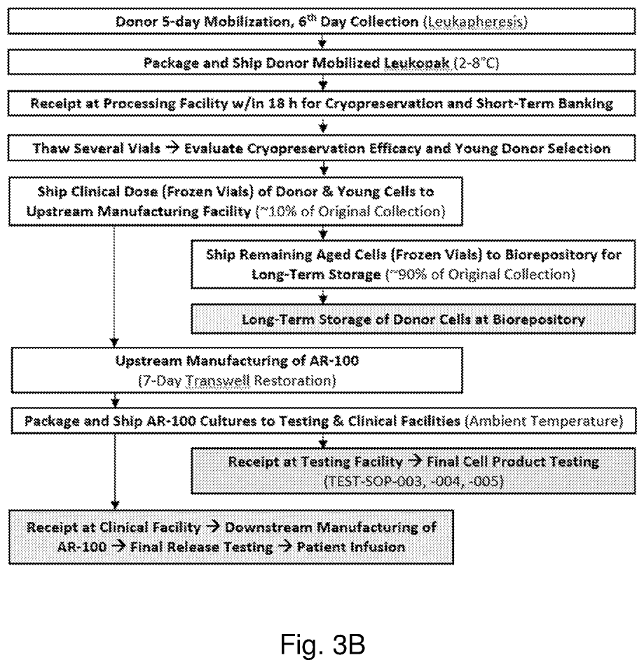

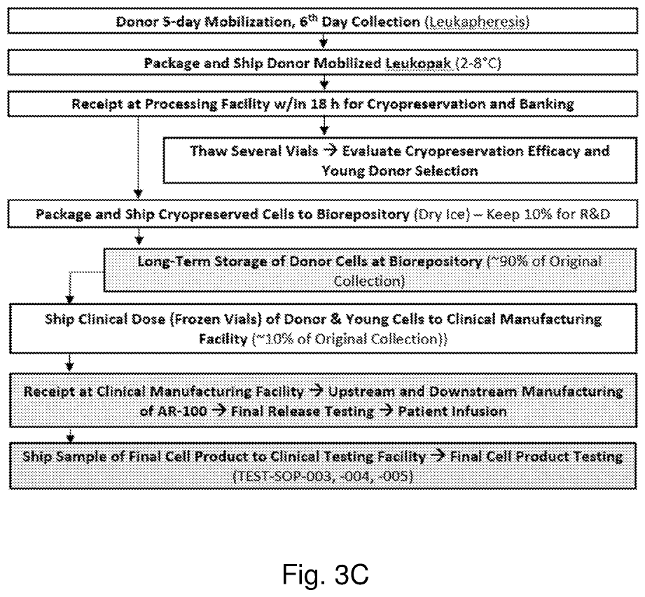

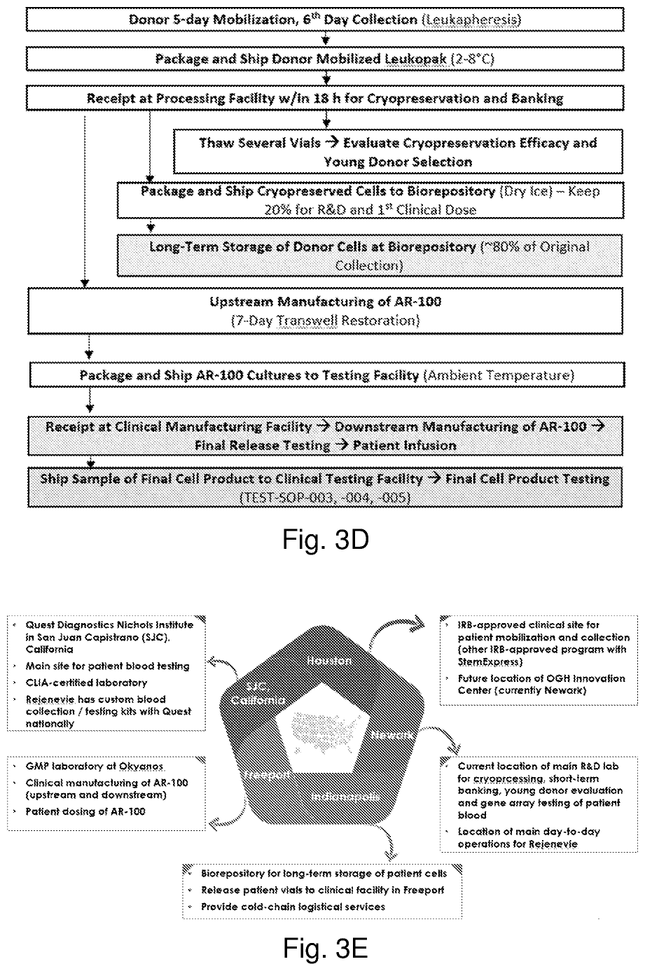

In some embodiments, the method of providing a target cell includes one or more of the steps as shown in FIG. 3A and as detailed in Examples 1-6. In some embodiments, mobilized cells are collected from a patient. In some embodiments, mobilized cells are collected from a donor and/or patient. In some embodiments, the mobilized cells are cryogenically preserved for a period of time at a storage facility. In some embodiments, the mobilized cells are cryogenically stored for transport. In some embodiments, the cells are transported to a clinical facility. In some embodiments, the patient cells and donor cells are thawed in an equilibration medium. In some embodiments, the patient cells and donor cells are separately thawed in an equilibration medium. In some embodiments, the patient cells and donor cells are placed in a transwell apparatus where they are physically separated from each other. In some embodiments, the transwell apparatus contains a restoration medium and/or a restoration medium is added to the transwell apparatus. In some embodiments, the patient and donor the cells are incubated in the restoration medium for a period of time. In some embodiments, the restoration medium is changed at various times throughout the incubation period. In some embodiments, the restoration medium is changed periodically, for example, every 2 days, 3 days, 4 days, or ranges including and/or spanning the aforementioned values. After a period of exposure to factors from the donor cells, the patient cells are converted to target cells (and/or become target cells). In some embodiments, instead of or in addition to exposure to donor cells, the patient cells are treated with one or more microRNAs as disclosed elsewhere herein, small molecules as disclosed elsewhere herein, and/or combinations thereof. In some embodiments, the donor cells are removed (e.g., by removing the transwell divider of the transwell plate) and the target cells collected. In some embodiments, the target cells are washed extensively (e.g., rinsed 1, 2, 3, 4, 5, 10, or more times, or a range of times spanning and or including the aforementioned values) to remove residual factors from the donor cells. In some embodiments, the target cells are transported to a clinical facility. In some embodiments, the target cells are prepared at the clinical facility. In some embodiments, the target cells are placed in an infusion medium. In some embodiments, the target cells are infused into the patient using an infusion medium. In some embodiments, the patient is tested periodically for improved health or adverse reactions.

In some embodiments, health screening is performed on a would-be patient (e.g., a potential patient). In some embodiments, health screening is performed on a would-be donor. In some embodiments, based on the health screening the would-be patient and/or the would-be donor either may be excluded or included from additional steps in the method. In some embodiments, the health screening is performed using a kit as disclosed elsewhere herein.

In some embodiments, once an acceptable patient and/or donor is found, one or more of the following steps is performed. In some embodiments, cells in the patient and/or donor are mobilized for a period of time (FIG. 3A.1). In some embodiments, after mobilization, blood is collected from the patient and/or donor. In some embodiments, the mobilized patient and/or donor cells are collected (e.g., with leukapheresis) as a leukopak. In some embodiments, during leukopak collection, plasma and blood cells in collected from the blood are reduced to provide the leukopak. In some embodiments, the locations for collection of one or more of the leukopak, for storage of the donor and patient cells (e.g., the leukopaks), for production of the target cell or cells, for administration of the target cells to a patient may be different. In some embodiments, the donor cells, patient cells, and/or target cells must be transported (e.g., to another location to generate the target cells or for the target cells to be administered to a patient). In some embodiments, the donor cells, patient cells, and/or target cells are packaged and shipped (at reduced temperature of less than or equal to about: 10° C., 8° C., 6° C., 4° C., 2° C., 0° C., or ranges spanning and/or including the aforementioned values) to a processing facility. In some embodiments, plasma is removed from the mobilized cells (e.g., greater than or equal to about: 25% of the plasma, 50% of the plasma, 75% of the plasma, or ranges spanning and/or including the aforementioned values). In some embodiments, the cells are collected from the leukopak and mixed in a cryogenic medium. In some embodiments, the patient cells, donor cells, and/or target cells are stored as a frozen mixture for a period of time. In some embodiments, the patient and/or donor cells are transported to a laboratory facility where the transwell process is performed (disclosed elsewhere herein).

In some embodiments, provided herein is a clinical-grade culture protocol that utilizes, for example, mobilized cells, blood stem cells, and/or immune cells collected from donors (e.g., healthy young donors) to restore function to mobilized cells, blood stem, and/or immune cells collected from patients (e.g., healthy aged patients) using a transwell culture. In some embodiments, a transwell culture apparatus allows factors released from the donor cells to permeate the transwell membrane and interact with the patient cells (e.g., located in the bottom chamber). In some embodiments, exposure of the patient cells to the donor milieu is performed for a period of time (e.g., a number of days that is less than or equal to 5, 6, 7, 8, 10, or ranges including and/or spanning the aforementioned values). In some embodiments, the exposure increases stem cell and immune function in the patient cells, which can then be washed and prepared for infusion as an cell therapy (e.g., autologous) for the patient (e.g., back into the patient). In some embodiments, the methods provided herein can be performed using only human compatible media and/or media with only human-derived factors and reagents which can include, for example, human-based serum. In some embodiments, the donor and the patient are the same person at different times. In some embodiments, the donor is a different person than the patient.

In some embodiments, prior to collection of donor cells, a potential donor is screened to determine if the he or she meets certain selection criteria and does not meet certain exclusion criteria. In some embodiments, if the potential donor does not meet each selection criteria or does meet the exclusion criteria, the potential donor can be excluded from donating cells. In some embodiments, if the potential donor meets each selection criteria or does not meet each exclusion criteria, the potential donor can be included as a donor to supply donor cells. In some embodiments, the donor inclusion criteria includes one or more of: Normal pulse (without irregularities) and in the range of 50 to 100 beats per minute; Normal blood pressure. Participants will be further evaluated for inclusion by the PI under the following conditions: a. Systolic pressure of <100 or >160 mm Hg; Diastolic pressure of <60 or >90 mm Hg; Results of urinalysis and basic chemistry panel performed during prescreening within normal limits; WBC >4.1×103/μL; % mononuclear cells (monocytes and lymphocytes): 15-55%; Absolute lymphocyte count: >0.60×103/μL; Test negative for the following infectious disease markers: HIV, Hepatitis B and C, HTLV and syphilis; 18-29 years old; Healthy and feeling well; Normal BMI (18.5-25); Weigh at least 120 lbs; Vaccination record current; Successful Leukopak donation; Meet protocol specifications, i.e. CBC (complete blood count) lab test; At least 5 days/week of moderate to strenuous exercise (minimum of 30 min); Successful completion of physical examination; Non-smoker; Between 5-15% body fat for men; 12.5-25% body fat for women; Healthy eating habits/diet with consumption of fish 2× per week regularly; Obtains 6-8 hours of sleep per night on a regular basis; and/or Have adequate peripheral veins for apheresis. In some embodiments, the donor exclusion criteria includes one or more of: Pregnant or breastfeeding; Current bleeding disorder, or history of bleeding disorders; History of hemoglobinopathy (e.g. sickle cell disease, thalassemia); History of myelodysplastic disorder; Autoimmune disease; Temperature >99.5° C.; Hemoglobin <12.5 g/dL or Hematocrit <38%; Platelet count <150×10³/μL; and Absolute neutrophil count <1500/μL Current or recent (<30 days) illness; Abnormal BMI (underweight, overweight, obese); Diet consisting of fast food more than once per week; Moderate to heavy regular alcohol consumption; pregnant; Prior cancer diagnosis; Previously mobilized; HIV, HPV, HBV or HCV positive test; History of heart, lung, liver, kidney disease; Blood or bleeding disorders; Neurologic disorders; Diabetes; and/or Autoimmune disorders.

In some embodiments, prior to collection of patient cells, a potential patient is screened to determine if the he or she meets certain selection criteria and does not meet certain exclusion criteria. In some embodiments, if the potential patient does not meet each selection criteria or does meet the exclusion criteria, the potential patient can be excluded from treatment. In some embodiments, if the potential patient meets each selection criteria or does not meet each exclusion criteria, the potential patient can be included in the methods of treatment disclosed herein. In some embodiments, the patient inclusion criteria includes one or more of: ≥60 years old; Healthy and feeling well; BMI of 18.5-29; Weigh at least 140 lbs; Successful Leukopak donation; Meet protocol specifications, i.e. CBC lab test; Vaccination record current; Have adequate peripheral veins for apheresis; Review and sign an IRB-approved procedure-specific consent form prior to the collection; Fill out donor history questionnaire; and/or Non-smoker. In some embodiments, the patient exclusion criteria includes one or more of: Current or recent (<30 days) illness; Underweight (<18.5) or Obese (>29) BMI; pregnant; Prior cancer diagnosis; Previously mobilized; HIV, HPV, HBV or HCV positive test; History of heart, lung, liver, kidney disease; Blood or bleeding disorders; Neurologic disorders; Diabetes; and/or Autoimmune disorders.

In some embodiments, donors and/or patients have peripheral blood collected prior to mobilization for baseline CBC, immune cell phenotyping and stimulation response (see methodology). In some embodiments, the study is longitudinal, with efficacy determined by comparison of efficacy measures at 2, 6, 12, and 24 months post-treatment to baseline (pre-treatment). In some embodiments, the baseline testing is performed no longer than 30 days prior to the first treatment of a patient with target cells.

In some embodiments, as disclosed elsewhere herein, methods of preparing target cells for treatment of patients are disclosed. In some embodiments, disclosed herein are human protocols utilizing factors produced by young healthy blood stem cells and immune cells to restore function to the aging blood and immune systems. While research-grade protocols attempted to restore the function of aging stem cells, disclosed herein are embodiments that utilize an autologous therapy that is clinical-grade. In some embodiments, cGMP principles are disclosed herein to generate donor, patient, and/or target cells as disclosed herein. In some embodiments, the protocol is substantially devoid of, devoid of, substantially lacks, and/or lacks any animal-derived products or supplements in the culture/restoration media. There are no comparative technologies that utilize young factors to restore function to the aging immune system as an autologous cell therapy for health aged patients. While some entities have transfused young plasma infusions into aged patients, this has now been outlawed in the US and lacked a scientific rationale for providing therapeutic benefit. This approach also lacked supportive preclinical data and carries unnecessary risk to the patient as this was a “non-self” therapeutic being infused into them (and has the capacity to transfer pathogens).

In some embodiments, the techniques for restoration and equilibration disclosed herein (and the restoration media as well as the equilibration media) yield safe and effective therapeutic protocols. In some embodiments, as disclosed elsewhere herein, the protocol is devoid of any animal-derived products. Thus, in some embodiments, both the restoration media and equilibration media or xeno-free and considered clinical-grade. A significant hurdle in translating these types of media formulations from preclinical to clinical is being able to reproduce efficacy when removing the classic media growth factors supplied by the use of fetal bovine serum (FBS). In some embodiments, the restoration protocol disclosed herein has replaced FBS with human serum albumin (HSA) that is commercially available, clinical-grade, and/or cGMP. In some embodiments, a number of other “animal” or “xeno” free culture media for growing human cells can be used. In some embodiments, the media comprises a base available from Stem Cell Technologies (StemSpan) and is further supplemented. Available animal-devoid medias have never been used in a clinical manufacturing process similar to that disclosed herein. In some embodiments, the restoration media and/or equilibration media may be supplemented with one or more of HSA, NEAA, IST, sodium pyruvate, glutamax, DNAse, Pen Strep, and/or glutamine at an amount (independently or collectively) of equal to or less than about: 0.1%, 0.5%, 0.75%, 1.5%, 5%, 8%, 12.5%, 15%, 25%, 50%, or ranges spanning and/or including the aforementioned values (by wt % or % by volume).

In some embodiments, the transwell process includes exposing patient cells to donor cells in a transwell system to prepare target cells. As disclosed elsewhere herein, in some embodiments, additionally or alternatively, the target cells are prepared by exposure to one or more microRNAs as disclosed elsewhere herein, small molecules as disclosed elsewhere herein, and/or combinations thereof. In some embodiments, restoration media is used during the transwell process (which may be a 7 day protocol or otherwise as disclosed herein). In some embodiments, the transwell process is performed for a period of time equal or less than 4 days, 6 days, 7 days, 8 days, 14 days, or ranges including and/or spanning the aforementioned values. In some embodiments, the restoration medium is used for producing the restored composition (e.g., a composition comprising target cells). In some embodiments, animal-devoid medium for blood and immune cells can be supplemented with one or more of human serum albumin, as well as other co-factors including: insulin, transferrin, selenium, sodium pyruvate, penicillin streptomycin, Glutamax and/or non-essential amino acids to provide the restoration media.

In some embodiments, frozen mobilized peripheral blood cells contains a complex mixture of immune cell types (e.g., patient or donor cells). Of note, neutrophils are notorious for not surviving the freeze-thaw process and lyse upon thawing. This lysis results in the release of DNA, which could easily cause the accumulation of viable cells to build up (clumping) and significantly reduce post-thaw yields (due to risks associated with clumping, such as stroke, etc.). In some embodiments, agents to prevent clumping are added to the equilibration medium. In some embodiments, an enzyme (e.g., DNaseI) is added into the equilibration media used to thaw cells. In some embodiments, this DNase I prevents clumping. In some embodiments, the DNaseI is of animal-free origin and thus can be considered a clinical-grade reagent for the batch. The equilibration media also contains RPMI as the base media, supplemented with human serum albumin, penicillin-streptomycin and Glutamax. In some embodiments, the equilibration media comprises Iscove's Modified Dulbecco's Medium (IMDM). In some embodiments, the equilibration media lacks Iscove's Modified Dulbecco's Medium (TMDM). In some embodiments, the equilibration media comprises Roswell Park Memorial Institute (RPMI). In some embodiments, the concentration of DNaseI added to the equilibration media directly affects whether a minimally turbid suspension is produced. In some embodiments, the thawed vials of cells are dropwise added into the equilibration medium.

In some embodiments, cells are allowed to equilibrate in equilibration media (e.g., at 37° C.) prior to addition to the transwell cultures. In some embodiments, the restoration media and equilibration media are used in other steps. In some embodiments, these could be used for second generation cell restoration approaches that do not utilize young cells, rather just the young factors that have been identified to be the mechanism of action (e.g., for micro RNAs and/or small molecules as disclosed elsewhere herein), in particular a combination of microRNAs. In some embodiments, the same equilibration and restoration medias would be utilized but for a gene therapy approach. In some embodiments, these media used for “scale-up” approaches where large numbers of transwell cultures would no longer be needed. In some embodiments, an apparatus would be similar to a single-use bioreactor. In some embodiments, these formulations could be utilized to restore the function of other aged non-immune cells and tissues, such as adipose-derived stem cells or stromal vascular fraction.

In some embodiments, the promotion of cellular health, as accomplished by the methods disclosed herein, may refer to alterations in parameters of cellular function that result in a perceived and/or quantifiable improvement in the viability state of cells and/or cell types. The viability state of a cell may be assessed using any suitable metric to evaluate parameters such as, but not limited to, cellular architecture, membrane organization and/or integrity, dynamic protein assemblies, molecular organization, and cellular responses to external signals. In some embodiments, the compositions and methods disclosed herein may improve the viability state of a cell as assessed by any suitable methodology. In some embodiments, a subject having improved and/or restored cellular function via the compositions and/or methodologies disclosed herein exhibits a perceived and/or quantifiable improvement in one or more aspects of the subject's cellular and/or general health. in some embodiments, the efficacy of the process can be measured through simple periodic blood draws post-treatment. In some embodiments, the processes disclosed herein can be used to treat one or more diseases linked to aging, including cancer, heart disease, stroke, Alzheimer's disease, and others. In some embodiments, the processes disclosed herein provide an immune restoration protocol for aging individuals to combat cancer and other age-related diseases.

In some embodiments, disclosed herein are methods comprising one or more steps of the following steps. In some embodiments, the method includes a step of obtaining a first cell sample from a first subject. In some embodiments, the method includes a step of obtaining a second cell sample from a second subject. In some embodiments, the method includes a step of culturing the first cell sample in culture media for a time period to produce a restoring medium. In some embodiments, the method includes a step of contacting the restoring medium with the second cell sample for a period of time to produce a target cell. In some embodiments, the method includes a step of administering the target cell to a patient in need of treatment.

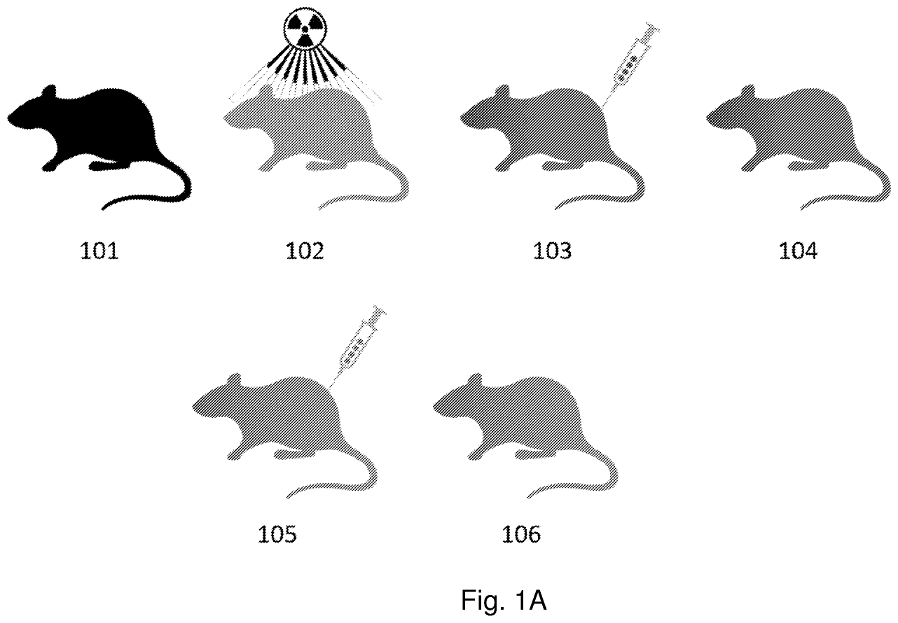

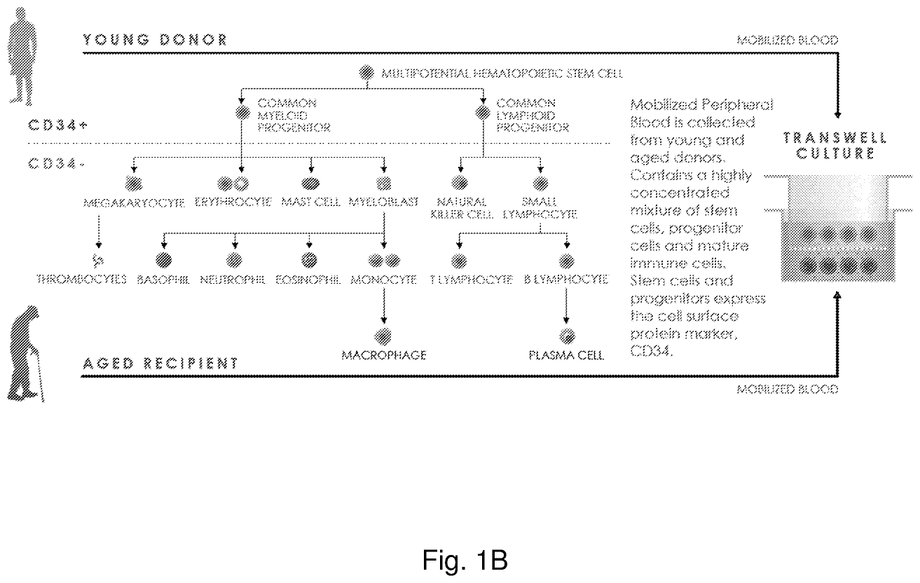

Shown in FIG. 1 is a schematic depicting an animal model for the therapeutic treatments disclosed herein. This example is performed using a NSG (NOD scid gamma) (101) mouse as a model patient. The NSG mouse is an immunodeficient laboratory mice of the strain NOD.Cg-Prkdc^{scid}Il2rg^{tm1Wjl}/SzJ1. NSG branded mice are among the most immunodeficient described to date. NSG branded mice lack mature T cells, B cells, and natural killer cells. The NSG mouse is exposed to sub-lethal radiation to clear out residual mouse immune cells (102). The NSG mouse was then exposed to aged human aged CD34+ stem and progenitor cells (103) at which point a humanized NSG mouse (104) results (where engrafted stem cells produce mature immune cells of the aged donor). The NSG mouse (104) acts as a model for autologous transplantation studies. At that time, the NSG mouse receives an autologous CD3-depleted restored aged cell infusion from a transwell culture (105). After 15 weeks, the infused mouse (106) shows that the process has no significant safety concerns. After 15 weeks, restored mice exhibited: increased blood T cell production and stimulatory activity, increased blood CD4/CD8 T cell ratio, decreased blood myeloid/lymphoid ratio, increased bone marrow hematopoietic stem cell function.

FIG. 1B depicts flow diagram providing an overview of one or more features of the disclosed treatment regimens. As shown, multipotent cells (including hematopoietic stem cells, myeloid progenitor cells, common lymphoid progenitor cells, CD34+ cells, etc.) can be harvested, enriched, and/or isolated from a young donor. Aged cells from a patient (which include multipotent cells, megakaryocytes, erythrocytes, mast cells myeloblasts, thrombocytes, basophils, neutrophils, eosinophils, monocytes, macrophages, natural killer cells, small lymphocytes, t-lymphocytes, B lymphocytes, plasma cells, CD34− cells, etc.) are exposed to the donor cells through a transwell culture, at which time the aged cells are changed by the factors from the donor cells to provide target cells. The target cells have one or more properties relative to their aged cell predecessors: increased capacity for proliferation, morphological changes, increased telomere lengths, lower S-β-GAL activity, lowered production of senescence-associated heterochromatic foci (SAHF), lowered production of senescence-associated secretory factors (SASF), lowered production of reactive oxygen species (ROS), lowered DNA damage, increased chaperone-mediated autophagy, or combinations thereof.

In some embodiments, as disclosed elsewhere herein, collection of mobilized mononuclear cell samples from healthy aged and young donors is performed. In some embodiments, a mobilizing agent is administered to participants (e.g., donors and/or patients). In some embodiments, participants are given an FDA approved, hematopoietic mobilizing agent on a daily basis at the currently recommended dosages (FIGS. 3A.2 and 4.2). In some embodiments, donors and/or patients are given a mobilizer (e.g., Filgrastim/Neupogen® (G-CSF)) at 5-10 ug/kg by subcutaneous injection daily (e.g., for about 5 consecutive days). In some embodiments, G-CSF stimulates the bone marrow to produce a large number of hematopoietic and progenitor stem cells and mobilizes them into the peripheral blood stream. In some embodiments, CBCs to assess the response to the mobilizing agent are performed prior to mobilization and on the final day of mobilization prior to mononuclear cell (MNC) collection. In some embodiments, on a following day (e.g., the 6^{th} day), mobilized peripheral blood MNCs are collected by leukapheresis using a cell separator (FIG. 3A.3). In some embodiments, during leukapheresis, the collection of plasma and red blood cells is controlled to lower the collection of plasma and red blood cells relative to other blood factors. In some embodiments, leukapheresis is performed (e.g., according to the manufacturer's instructions to process 18 L of blood at a flow rate of 50 to 100 mL per min). In some embodiments, mobilized MNC collections are performed for 4 to 6 hours for completion. In some embodiments, participants have only one MNC collection performed immediately following mobilization. The product of 1 full MNC collection is referred to as a Leukopak. Fresh leukopaks should be processed within 24 hours of collection and should be stored at room temperature.

In some embodiments, the leukapheresis is performed using one or more of the following steps. In some embodiments, prior to collection, one or more pieces of the following information is gathered: documentation of the date of signed informed consent, venous assessment, CBC within 30 days of proposed collection date, Infectious Disease Markers testing statement (formal notification of known positive viral markers or known relevant communicable disease agents and diseases). In some embodiments, the mobilizing agent is administered according to each patient's doctor's instruction. In some embodiments, the entity that administers the mobilizing agent uses a predictive algorithm to calculate the optimum Total Blood Volume (TBV) required in order to meet the requested Mononuclear Cell (MNC) cell dose is used.

In some embodiments, a typical number of MNCs harvested from the leukapheresis procedure range from 25×10⁹-50×10⁹ cells. In some embodiments, the MNCs have a viability >95% and a collection volume of 300-400 mL (approximately 100×10⁶ cells/mL). In some embodiments, prior to cell processing, a sample of the Leukopak should be collected and cell number determined by counting with a hemocytometer. In some embodiments, further, cell viability should be determined using Turk's solution (as disclosed elsewhere herein). Additional evaluation of expression for the biomarkers CD45 and CD34 in the MNC collection can be made by flow cytometry to determine the percentage of leukocytes and hematopoietic stem/progenitor cells, respectively. In some embodiments, cells can be selected for those markers or others. In some embodiments, equal to or greater than about half of the plasma collected is removed from the resulting blood product. Typical number of CD34⁺ cells collected from mobilized leukapheresis range from 1-2×10⁷ cells per harvest dependent on the age of the donor, with young donors demonstrating greater yield. Cells are diluted in cryopreservation media at a 1:1 ratio to yield a final cell suspension of approximately 50×10⁶ cells/mL containing human serum albumin (HSA) and DMSO. In some embodiments, cells are then frozen (FIGS. 3A.4 and 4.3) using a programmable controlled rate freezer at a rate of −1° C./min to a temperature of −100° C. for transfer to liquid nitrogen storage.

In some embodiments, as shown in FIGS. 3A.5 and 4.3-4.6, when mobilized peripheral blood (MPB) collections are scheduled, shipping logistics are in place for proper sample handling and for an unbroken cold chain from collection to long-term storage. In some embodiments, a process to improve the viability of collected samples has been established. In some embodiments, blood sample shipments from the collection site to the cell processing site are performed using a validated cold storage cryoshippers (e.g., C3™ Shipper, or cold shipper, etc.). In some embodiments, data loggers (e.g., SmartPak II™) are used to monitor the cell temperature over the course of transport. In some embodiments, these cryoshippers may be provided in a kit as disclosed elsewhere herein.

In some embodiments, the cryoshipper (e.g., cold shipper) is a Controlled Cold Temperature Shipper for global and domestic 2-8° C. transport services. In some embodiments, the shipper can be outfitted with tracking system (e.g., SmartPak II™ data logging system—referred to SP II™ in the rest of the document). In some embodiments, the tracking system has onboard software which allows the user to monitor the shipper (e.g., temperature, location, etc.) during service. In some embodiments, important information and recorded data can be transmitted via cellular network to a central hub (e.g., the Cryoportal™—Cryoport's web-based order and tracking system) and displayed on a Live view for the client to monitor shipments when in-transit).

In some embodiments, the following outlines embodiments of the process and steps that can be carried out at the collection site for MPB collection and during shipment to the cell processing site. In some embodiments, the following outlines embodiments of the process and steps that can be carried out at the collection site for mobilized peripheral blood (MPB) collection and during shipment to the cell processing site. Also disclosed are one or more kit components that may be used.

The cryoshipper may comprise one or more of the following: cold shipper—C31296S (with SP II), ACC-9123—SmartPak II Data Logger (PT300D), 9151—Battery Pack, SmartPak II, 9223—Temperature Probe, 2-8, 9238—Phase Change Panels, C31296, 9228—Insert, TC Mount, C31296, 9231—Insert, Foam, SmartPak, C31296, 9187—SmartPak II Sleeve, 9225—Box, Inner, C31296 (Inner White Box), 9253—Box, Outer, C31296 (Outer Brown Box), ACC-9140-SafePak XL.

Collection site supplies the following: Each Donor Collection Bag/Leukopak—Quantity 1, Each Specimen Transport Bag (95 kPa Transport Bag)—Quantity 1, 37° C. incubator/waterbath, 2-8° C. Walk-In Refrigerator. Cryoport (or similar) supplies the following: Cryoport cold Shipper™ 2-8° C. Shipper—Quantity 1; Includes 6 Phase Change Panels (PCP) in the interior of the shipper (Appendix I); ACC-9035: Exempt Human Specimen Label—Quantity 1; ACC-9123: SmartPak II™ Condition Monitoring System (Data Logger)—Quantity 1; SRV-LIV: Live View for SmartPak II™—Quantity 1; ACC-9140: SafepakXL™ Tyvek bag for secondary packaging of the Payload—Quantity 1. These supplies may also be provided in a kit as disclosed elsewhere herein.

In some embodiments, a cold shipping vendor (e.g., Cryoport of the like) order form (e.g., a Cryoport Order Form) is used to request a cold shipping container (e.g., the cold Shipper™ Shipper). In some embodiments, the form should be completed and submitted directly to the shipping vendor's Customer Service Team at a time that results in on time delivery of the shipper (e.g., by about 2:00 PM Pacific Time on the day before leukophoresis before or earlier). In some embodiments, orders should be placed as far in advance as possible. In some embodiments, all details for each request should be completed prior to submission of the Order Form to the shipping vendor. In some embodiments, shipments for the collection site will originate from the shipping vendor (Cryoport Irvine, Calif. Logistics Center). Any one or more of these components may also be provided in a kit as disclosed elsewhere herein.

In some embodiments, the cryoshipper cold shipper is received (e.g., by about 10:30 am by Gulf Coast). In some embodiments, within an hour of receiving, the site should discard the outer packaging. In some embodiments, the cold shipper will then be contained in only the outer white packaging. In some embodiments, place cold shipper with the transport bag (e.g., SafePakXL) inside in a 5° C. (+/−3° C.) walk-in refrigerator for conditioning (e.g., overnight or for a period that allows temperature equilibration). In some embodiments, if the cold shipper is received the day prior to collection or is received the same day as collection, place it into the refrigerator for temperature equilibration. If a 95 kPa bag transport bag provided by Gulf Coast is preferred to the SafePakXL, then the 95 kPa bag transport bag should also be placed within a in a 5° C. (+/−3° C.) refrigerator for temperature conditioning (e.g., overnight) if the cold shipper is received the day prior to collection, or until loading if the cold shipper is received the same day as collection. Note date and time of transport bag placement into the refrigerator.

Some embodiments pertain to methods of preparing and preconditioning the payload. For instance, in some embodiments, after donor collection is completed (FIG. 3A.3), the transport bag (e.g., a SafePakXL, 95 kPa transport bag, or the like) is removed from the refrigerator. In some embodiments, the freshly collected donor collection bag (Leukopak) with a volume of approximately 300-400 mL is inserted immediately into a first bag (e.g., a 95 kPa Specimen Transport Bag) and sealed. In some embodiments, the sealed first bag (e.g., the Specimen Transport Bag) is inserted into a second container (e.g., a Safepak™XL Tyvek Bag, which may be included with the cold shipper) and secured by an adhesive seal. In some embodiments, the combined unit is referred to as the payload. In some embodiments, the payload is placed in a refrigerator unit set to 5° C. (+/−3° C.) for a minimum of 90 minutes. In some embodiments, refrigerating the donor collection is a temperature pre-conditioning step. In some embodiments, this will slowly bring the blood temperature to the desired transport temperature prior to being inserted into the cold shipper unit. In some embodiments, these steps prevent the body temperature Leukopak from warming the 2-8° C. cold shipper chamber upon insertion and decreasing the temperature stability of the vessel.

Some embodiments include loading the payload and specialty courier pickup of the loaded cold shipper. In some embodiments, the cold shipper is removed from cold storage. In some embodiments, the cold shipper is removed from cold storage at a time that is near or as close to loading time as possible (e.g., less than or equal to about 60 minutes prior to pickup). Note date and time that cold shipper was removed from walk-in refrigerator. In some embodiments, the payload is removed from the refrigerator as close to loading time as possible, and no earlier than 90 minutes prior to initial placement in the refrigerator. Note date and time that payload was removed from walk-in refrigerator. In some embodiments, the cold shipper white box is opened to expose the silver cooler lid. In some embodiments, using the appropriate PPE for cold temperatures, remove the silver cooler lid. In some embodiments, the top white colored plastic Phase Change Panel (PCP) is removed to access the specimen chamber. In some embodiments, the payload is placed into the pre-conditioned cold shipper and readied for shipment. In some embodiments, once the payload is inserted, replace the PCP, then the silver cooler lid. Close the cold Shipper™ Shipper outer packaging.

In some embodiments, Close Flap B first, then Flap A on top. All shipping documents required for the shipment leg to the Manufacturing Facility will already be enclosed in a shipping envelope on Flap A. In some embodiments, attach an Exempt Human Specimen Label to Flap A. In some embodiments, tape the box securely with clear shipping tape to seal the flaps for shipment.

In some embodiments, the cold shipper containing the Payload may then be placed back in a walk-in refrigerator until ready for pickup. Note date and time that cold shipper containing payload was placed back into walk-in refrigerator. In some embodiments, prior arrangements will have been made for a specialty courier to pick up on the day of collection at a fixed time (e.g., by about 4:00 PM local time). In some embodiments, the shipping vendor can arrange for pickup in the evening (e.g., as late as 7 PM) if extenuating circumstances require. In some embodiments, the pick-up can be as late as allows overnight delivery (e.g., the cutoff to deliver the Payload to the plane for overnight transport, etc.).

In some embodiments, the pick-up arrangement will be at the discretion of the site, depending on when daily operations close. In some embodiments, target pickup time will be afternoon (e.g., about 4:00 PM local time or earlier). In some embodiments, the shipping vendor is informed the day before to schedule specialty courier pick up time. In some embodiments, request for changes to the shipment schedule should occur at least 3 hours prior to the scheduled pickup time when possible, however changes can may be made up to 1 hour prior if required. In some embodiments, the shipping vendor can attempt to accommodate all schedule change requests to ensure integrity and receipt of the Payload to the Manufacturing Facility.

In some embodiments, about thirty (30) minutes prior to scheduled pickup, the cold shipper containing the pre-conditioned payload is taken out of the walk-in refrigerator and placed in the outbound shipping area. In some embodiments, the cold shipper containing the payload should then be placed at ambient temperature in the area where pickups occur and await pickup by specialty carrier (e.g., Bluebird Bio). In some embodiments, the shipping vendor will manage and arrange all pickups. In some embodiments, the Specialty Courier will arrive at the pre-arranged time, pick up the cold shipper and will ship to the Manufacturing Facility at the address specified below.

Receipt of cold shipper Containing Payload. In some embodiments, delivery of the payload to the manufacturing site will be sent via email notification from the shipping vendor. In some embodiments, staff at the manufacturing site will intercept payload from the loading dock and transport to the cryoprocessing lab. Note date and time that payload was received in the cryoprocessing lab. In some embodiments, staff will begin cryoprocessing the payload for storage in liquid nitrogen and banking.

In some embodiments, the following are Acceptance Criteria for the leukopak: In some embodiments, all contents of the shipper remain within the range of 2-8° C. Shipper Hold Time Analysis—Cold Shipper™ meets hold time requirements when stored at 4° C. overnight. In some embodiments, for collections in which the cold shipper arrives the day prior to donor collection, the cold shipper is conditioned overnight in a walk-in refrigerator. In some embodiments, this allows proper temperature stability throughout all legs of the process. In some embodiments, if the clinical site fails to precondition the cold shipper overnight, proper temperature stability should still be maintained throughout all legs of the process; as demonstrated by the protocol validation run without cold shipper and payload preconditioning.

In some embodiments, following donor mobilized blood collection, the blood bag should be inserted into the pre-conditioned transport bag and the payload placed in a refrigerator unit set to 5° C. (+/−3° C.) for a minimum of 90 minutes. In some embodiments, this allows proper temperature stability throughout all legs of the process.

In some embodiments, target pickup time for the cold shipper containing the payload is a time that allows for over night shipping (e.g., about 1:00 PM, 4:00 PM, 7:00 PM, etc.) to allow overnight delivery. If the cutoff time is missed due to an error either with the specialty courier or with the clinical site (for instance a donor collection ran late), then the cold shipper containing payload is to be placed in the walk-in refrigerator overnight.

In some embodiments, for same day pickup and delivery, the cold shipper containing the payload is to be picked up by the specialty courier at a time that allows delivery that day to the manufacturing site (e.g., 7 AM local time, as there is one flight arriving at Newark at 4:40 PM local time on the same day). In some embodiments, where the delivery site is Newark, delivery to the manufacturing site would be around 6:30 PM local time.

Deviation #4—if the cold shipper does not Arrive at Specified Time for Same-Day Donor Collections. In some embodiments, if a donor is schedule for mobilized blood collection on a Tuesday, Cryoport will ship the cold shipper on Monday for Tuesday delivery to the clinical site by morning (e.g., about 10:30 AM local time via Fedex Priority Overnight service). In some embodiments, if the cold shipper does not arrive at the clinical site by mid morning (about 11:30 AM local time in the above scenario), the clinical site can immediately notify the shipping vendor's customer service team of the late delivery via email (e.g., to cs@cryoport.com) for the shipping vendor to facilitate a corrective action.

Deviation #5—A Transit Error Occurs that Impedes Timely Delivery to the Manufacturing Site. In some embodiments, if the error occurs at the level of the specialty courier such that delivery to the plane prior to cutoff is not possible. The courier should return the cold shipper containing the payload to the clinical site and a shipping sequence presented above initiated. If the clinical site is no longer open for business, then arrangements will be made by the shipping vendor to initiate a sequence as presented above. The shipping vendor would immediately be notified of this deviation and would employ their own corrective actions to ensure this sequence occurs. If the error occurs at the level of the aircraft such that timely delivery to the manufacturing site is not possible then then arrangements will be made by the shipping vendor to initiate a sequence presented above should be initiated (e.g., storage overnight in an appropriate freezer). the shipping vendor would immediately be notified of this deviation and would employ their own corrective actions to ensure this sequence occurs.

An overview of embodiments of shipper specifications follows: Shipper contents: Remain within 2-8° C., Product is not damaged during transport, Product remains viable for therapy, Assessment of carrier performance: Stated pick up, delivery and transport times, No damage occurs to shipper during transit, Validate the shipping documentation is adequate to enable pickup and delivery to and from sites, Validate the packaging labeling: Reflects the contents, Does not cause any delays in shipping. Test Shipments (2)—from Site (Houston, Tex.) to manufacturing facility (Newark, N.J.); Scenario 1: Shipper placed in 2-8° C. refrigerator upon receipt, Patient sample pre-conditioned; Scenario 2: Shipper left at ambient temp upon receipt, Patient sample pre-conditioned.

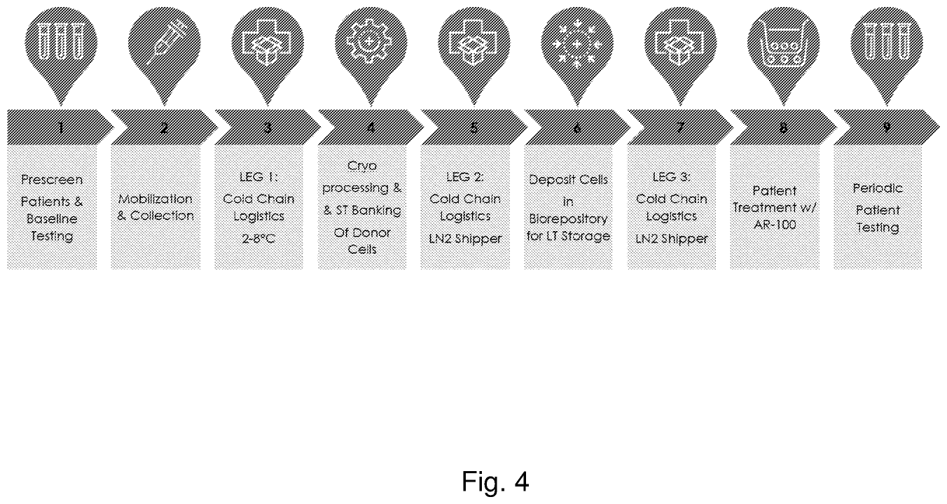

The following describes the actions taken at FIG. 3A.5 (and FIG. 4.6) for long term storage of aged donor cells. In some embodiments, initial collections will include aged and young individuals for stem cell mobilization and leukapheresis (as described above). This section discloses procedures for use in the cryogenic processing of G-CSF-mobilized patient Leukopaks. In some embodiments, one or more of the following steps improve cell yield and viability, while allowing compatibility with regulatory guidelines and medical application. All procedures described in this section are for mobilized peripheral blood cells from aged donors (CRYO-MBR-A). One or more of the following steps may be omitted.

Making Cryogenic Media for Total Nucleated Cells (TNCs): In some embodiments, a cryogenic medium is prepared. In some embodiments, a cryogenic medium is prepared using HSA, DMSO, and normal saline.

Addition of Cryogenic Media to TNCs from Mobilized Peripheral Blood (MPBs): In some embodiments, a centrifuge is equilibrated to 4° C. before processing the Leukopaks. In some embodiments, label cryogenic vials with Date, Patient ID, Vial Number, Patient Initials (patient descriptor, patient ID #, tube #, patient initials). For example: 12/05/18 PT-004-001-AC. In some embodiments, in ascending order, organize the cryogenic vial numbers into clean racks. Label cryogenic boxes with “rack number” location and “box number” (R# B#). In some embodiments, turn and leave on laminar flow hood, sterilize working surfaces with 70% ethanol and UV for a minimum of 10 minutes. In some embodiments, place labeled cryogenic boxes and labeled cryogenic vials under the laminar flow hood then turn on UV for a minimum of 20 minutes. In some embodiments, place the cryogenic vials on their designated boxes (e.g., 5 mL cryogenic vials should be placed into 5 mL cryogenic boxes, and 2 mL cryogenic vials should be placed into 2 mL cryogenic boxes). In some embodiments, place respective labeled cryogenic boxes (e.g., containing the respective labeled cryogenic vials) in the fridge (e.g., for 15 minutes or more). In some embodiments, ethanol spray and wipe down the chilled bead bucket and place under the laminar flow hood.

In some embodiments, the Leukopaks are removed from the shipping container and sprayed with ethanol thoroughly. In some embodiments, the Leukopaks are wiped down and placed under the sterile laminar flow hood. In some embodiments, with sterile scissors, cut the top portion of the Leukopaks and transfer a portion of the contents (e.g., equivalent to 1/10th of total Leukopak volume of MPBs each) into 10×50 mL sterile conical tubes. In some embodiments, spin down to pellet (e.g., at 300 g, at 4° C., for 10 minutes). In some embodiments, from each conical tube, remove 50% (approximately 20 mL per conical tube) of supernatant from each of the 10×50 mL conical tubes until left with only 50% of the initial total volume of supernatant and cell pellets. In some embodiments, with a 50 mL pipet and without disturbing the cell pellet, remove the remaining 20 mL supernatant and place it in a sterile 250 mL bottle. In some embodiments, loosen the pellets for all 10×50 mL conical tubes (e.g., with light tapping). In some embodiments, carefully resuspend pellets with 20 mL supernatant. In some embodiments, transfer the cell suspension into a single, sterile 500 mL bottle (total volume should be 200 mL). Keep the cell suspension chilled by placing the 500 mL bottle in the bucket with the cold beads.

In some embodiments, drop-wise add 200 mL of chilled cryogenic media into the cell suspension while gently shaking the bottle.

Aliquoting of Aged TNCs from Mobilized Peripheral Blood (MPBs) for long term storage and research and development (R&D): 90% of the leukopak+cryogenic media, here in referred to as cryogenic suspension will be allocated for long term storage while 10% of this solution will be allocated for young donor evaluation.

90% of cryogenic suspension=360 mL; 72×5 mL vials

10% of cryogenic suspension=40 mL; 20×2 mL vials

In some embodiments, after gently mixing the cells with the cryogenic media, take a 1 mL aliquot and place at 4° C. for cell counting. In some embodiments, aliquot 90% of the cryogenic suspension into 5 mL aliquots within cryogenic vials. In some embodiments, place vials back into designated (5 mL cryogenic vials should be placed into 5 mL cryogenic boxes) boxes and place at 4° C. for 15 minutes. In some embodiments, aliquot 10% of the cryogenic suspension into 2 mL aliquots within cryogenic vials. In some embodiments, place vials back into designated boxes (2 mL cryogenic vials should be placed into 2 mL cryogenic boxes) and place at 4° C. for 15 minutes. Next, transfer vials from the previous steps, to the controlled rate freezer and the following programed protocol.

Cryogenic Freezing of Cells: In some embodiments, set the Controlled-rate Freezing (CRF) program to an appropriate setting to achieve cryogenic freezing. In some embodiments, the temperature in the CRF chamber should be programmed to different temperature and cooling rates. In some embodiments, a controlled rate freezing program is utilized. In some embodiments, the program is custom and carried out with a controlled rate freezer to freeze the cells at an average rate of −1 degrees C. per minute from a starting temperature of 2-8 degrees C. to −100 degrees C. prior to deposition into a liquid nitrogen dewar.

In some embodiments, after the freezing, immediately transfer the boxes of cryogenic vials into the vapor phase of liquid nitrogen tank for long term cryopreservation. In some embodiments, perform cell count with Turk's solution and Trypan Blue exclusion. Record cell number, volume, concentration and viability. Between a minimum of 72 h and a maximum of 144 h, 1 vial of banked 5 mL cells should be thawed from donor sample to be tested.

Long Term Storage (Of Cells From Young Donors): The following describes the actions taken at FIG. 3A.5 (and FIG. 4.6) for long term storage of young donor cells. In some embodiments, initial collections will include aged and young individuals for stem cell mobilization and leukapheresis (as described above). This section discloses procedures for use in the cryogenic processing of G-CSF-mobilized patient Leukopaks. In some embodiments, one or more of the following steps improve cell yield and viability, while allowing compatibility with regulatory guidelines and medical application. All procedures described in this section are for mobilized peripheral blood cells from young donors (CRYO-MBR-Y). One or more of the following steps may be omitted.

To make about 250 mL of cryogenic media (MED-CRYO-100+), to a 500 mL sterile bottle add Normal Saline; HSA; and DMSO. Place solution at 4° C. until ready to be used.

In some embodiments, a centrifuge is equilibrated to 4° C. before processing the Leukopaks. In some embodiments, label cryogenic vials with Date, Donor ID, Vial Number, Patient Initials (patient descriptor, patient ID #, tube #, patient initials). For example: 12/16/18 DN-006-001-KG. In some embodiments, in ascending order, organize the cryogenic vial numbers into clean racks. Label cryogenic boxes with “rack number” location and “box number” (R# B#). In some embodiments, turn and leave on laminar flow hood, sterilize working surfaces with 70% ethanol and UV for a minimum of 10 minutes. In some embodiments, place labeled cryogenic boxes and labeled cryogenic vials under the laminar flow hood then turn on UV for a minimum of 20 minutes. In some embodiments, place the cryogenic vials on their designated boxes (e.g., 5 mL cryogenic vials should be placed into 5 mL cryogenic boxes, and 2 mL cryogenic vials should be placed into 2 mL cryogenic boxes). In some embodiments, place respective labeled cryogenic boxes (e.g., containing the respective labeled cryogenic vials) in the fridge (e.g., for 15 minutes or more). In some embodiments, ethanol spray and wipe down the chilled bead bucket and place under the laminar flow hood.

In some embodiments, the Leukopaks are removed from the shipping container and sprayed with ethanol thoroughly. In some embodiments, the Leukopaks are wiped down and placed under the sterile laminar flow hood. In some embodiments, with sterile scissors, cut the top portion of the Leukopaks and transfer a portion of the contents (e.g., equivalent to 1/10th of total Leukopak volume of MPBs each) into 10×50 mL sterile conical tubes. In some embodiments, spin down to pellet (e.g., at 300 g, at 4° C., for 10 minutes). In some embodiments, from each conical tube, remove 50% (approximately 20 mL per conical tube) of supernatant from each of the 10×50 mL conical tubes until left with only 50% of the initial total volume of supernatant and cell pellets. In some embodiments, with a 50 mL pipet and without disturbing the cell pellet, remove the remaining 20 mL supernatant and place it in a sterile 250 mL bottle. In some embodiments, loosen the pellets for all 10×50 mL conical tubes (e.g., with light tapping). In some embodiments, carefully resuspend pellets with 20 mL supernatant. In some embodiments, transfer the cell suspension into a single, sterile 500 mL bottle (total volume should be 200 mL). Keep the cell suspension chilled by placing the 500 mL bottle in the bucket with the cold beads.

In some embodiments, drop-wise add 200 mL of chilled cryogenic media into the cell suspension while gently shaking the bottle.

Some embodiments for specifications associated with Leukopaks include or exclude one or more of the following. I. Deliverables by StemExpress to Customer: 1. Fresh Mobilized Leukopak; Collected from donors sent to StemExpress by Customer; Dosing regimen of daily G-CSF (Neupogen) injections at a dose of 10 g/kg/day for 5 consecutive days, with leukapheresis collection on the 6^{th} day; Shipped in temperature-controlled packaging maintained between 2-8° C., and received by Customer within 24 hours of collection by FedEx First Overnight service or equivalent. 2. Certificate of Analysis a. To be sent electronically or via hard copy, and delivered prior to arrival of or with the Leukopak, respectively b. Should contain the following information i. Donor specification Age, Sex, Height, Weight, Ethnicity, Donor ID#. ii. Procedural specification: Needle IN time, Needle OUT time, Iii. Product specification: Total collection volume, Total nucleated cell count, Total nucleated cell viability, Percentage of CD45+ cells, Percentage of CD34+ cells, Low hematocrit, Low granulocytes. II. Acceptable Ranges for Product Specifications 1. Volume a. Minimum: 300 mL b. Maximum: 500 mL 2. Total Nucleated Cell Count a. Minimum: 20×10⁹ cells b. Maximum: None 3. Total Nucleated Cell Viability a. Minimum: 90% b. Maximum: 100% 4. Percentage of CD34+ cells a. Minimum: 1% b. Maximum: None.

90% of the leukopak+cryogenic media, here in referred to as cryogenic suspension will be allocated for long term storage while 10% of this solution will be allocated for young donor evaluation.

90% of cryogenic suspension=320 mL; 64×5 mL vials

10% of cryogenic suspension=80 mL; 40×2 mL vials

In some embodiments, after gently mixing the cells with the cryogenic media, take a 1 mL aliquot and place at 4° C. for cell counting. In some embodiments, aliquot 90% of the cryogenic suspension into 5 mL aliquots within cryogenic vials. In some embodiments, place vials back into designated (5 mL cryogenic vials should be placed into 5 mL cryogenic boxes) boxes and place at 4° C. for 15 minutes. In some embodiments, aliquot 10% of the cryogenic suspension into 2 mL aliquots within cryogenic vials. In some embodiments, place vials back into designated boxes (2 mL cryogenic vials should be placed into 2 mL cryogenic boxes) and place at 4° C. for 15 minutes. Next, transfer vials from the previous steps, to the controlled rate freezer and the following programed protocol.

In some embodiments, set the Controlled-rate Freezing (CRF) program to an appropriate setting to achieve cryogenic freezing. In some embodiments, the temperature in the CRF chamber should be programmed to different temperature and cooling rates. In some embodiments, a controlled rate freezing program is utilized. In some embodiments, the program is custom and carried out with a controlled rate freezer to freeze the cells at an average rate of −1 degrees C. per minute from a starting temperature of 2-8 degrees C. to −100 degrees C. prior to deposition into a liquid nitrogen dewar.

In some embodiments, after the freezing, immediately transfer the boxes of cryogenic vials into the vapor phase of liquid nitrogen tank for long term cryopreservation. In some embodiments, perform cell count with Turk's solution and Trypan Blue exclusion. Record cell number, volume, concentration and viability. Between a minimum of 72 h and a maximum of 144 h, 1 vial of banked 5 mL cells should be thawed from donor sample to be tested.

In some embodiments, long term storage (e.g., for donor, patient, or target cells) is performed at a temperature of equal to or less than about: −100° C., −150° C., −180° C., −190° C., −200° C., or ranges spanning and/or including the aforementioned values.

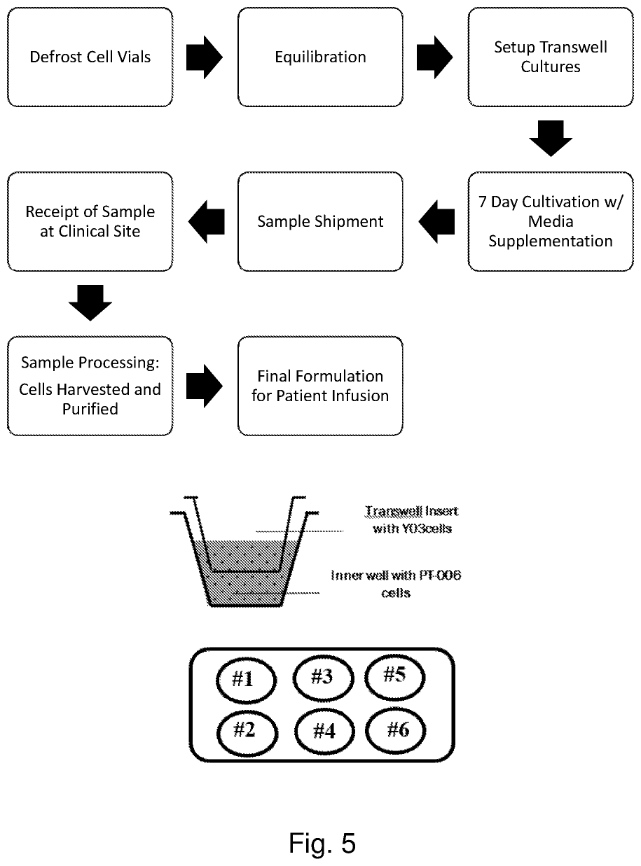

The following describes the treatment of aged cells with donor cells as shown in FIGS. 3A.6-3A.9 and 4.8. FIG. 5 provides an alternative depiction of the treatment steps, one or more of which may be omitted. For FIG. 5, a general schematic workflow illustrating the cell restoration process, including shipment, the entire process may take as little as 8 days. In some embodiments, the first 5 steps are at cell production facility, last 3 steps are at clinical facility. In some embodiments, the aged cells cultured with donor cells in the transwell are referred to as the composition AR-100.

Cell production, processing and clinical infusion: This example describes embodiments for the production of therapeutic cells for infusion into a patient, as shown in FIGS. 3A.6-3A.10 and 4.8-4.9. In some embodiments, the protocol utilizes cells from an aged donor and a young donor, where the aged donor is the patient and young donor is the donor. In some embodiments, cells produced are solely for infusion into patient.

Equilibration Media: In some embodiments, 400 mL of equilibration media for young total nucleated cells is prepared. In some embodiments, supplemented Roswell Park Memorial Institute media (RPMI) is prepared. In some embodiments, to prepare Roswell Park Memorial Institute media (RPMI) supplemented with Penicillin Streptomycin & GlutaMAX-I, one or more of the following steps are used. In some embodiments, add of Penicillin Streptomycin and GlutaMAX-I to RPMI media bottle. In some embodiments, prepare DNase I and add. In some embodiments, place at −80° C. until ready to use then thaw overnight at 4° C. In some embodiments, place the 1 L bottle of media at 37° C. for 15 minutes.

In some embodiments, the equilibration medium is prepared using one or more of the following procedures. Section 1: Step 1: Supplementing RPMI with Pen Strep & Glutamine. Step 2: Dissolving DNAse in sterile water. Sterile filter using a 10 mL syringe and 0.2 um filter into a new sterile 15 mL conical tube. To each sterile 1 L disposable bottle add: H.S.A., RPMI (made in Step 1, section 1), DNAse I solution (made in Step 2, section 1) Step 4: Place the 1 L bottles of media at 37° C. for 15 minutes.

Equilibration Media. In some embodiments, 650 mL of RPMI equilibration media for PT-006 cells is prepared. In some embodiments, the RPMI is supplemented with Penicillin Streptomycin & GlutaMAX-I. In some embodiments, DNase is dissolved in sterile water: In some embodiments, sterile filter (e.g., using a 10 mL syringe and 0.2 μm filter) into a new sterile 15 mL conical tube. In some embodiments, place at −20° C. until ready to use then thaw overnight at 4° C. In some embodiments, to one sterile 1 L disposable bottle add: H.S.A.; RPMI; DNase I solution. In some embodiments, place the 1 L bottles of media at 37° C. for 15 minutes.

In some embodiments, the equilibration medium is prepared using one or more of the following procedures. SECTION 2: 1000 mL of equilibration media for B0 TNCs Step 1: Supplementing RPMI with Pen Strep & Glutamine: Pen Strep and Glutamine to RPMI media bottle. Step 2: Dissolving DNAse in sterile water, Sterile filter using a 10 mL syringe and 0.2 um filter into a new sterile 15 mL conical tube. Step 3: To each sterile 1 L disposable bottle (2 total, approx. 500 mL/bottle) add: H.S.A. RPMI (made in Step 1, section 2) DNAse I solution (made in Step 2, section 2) Step 4: Place the 1 L bottles of media at 37° C. for 15 minutes.

In some embodiments, cell restoration media is prepared. In some embodiments, to each Stem Span media bottle (2 total) add Penicillin Streptomycin and GlutaMAX-I. In some embodiments, place at 4° C. until ready to be used. In some embodiments, to make 1000 mL of cell restoration media, herein defined by the designation MED-CR-100+, to each MED-CR-100 bottle add MEM Non-Essential Amino Acids Solution, Insulin-Transferrin-Selenium-Sodium Pyruvate, H.S.A. Place at 4° C. until ready to be used.

In some embodiments, the restoration medium is prepared using one or more of the following procedures. In some embodiments, to Stem Span media bottle add Pen Strep and Glutamine. Place at 4° C. until ready to be used. To each bottle add: MEM Non-Essential Amino Acids Solution (100×) Insulin-Transferrin-Selenium-Sodium Pyruvate (ITS-A) (100×) H.S.A.

Some embodiments include steps for defrosting cells and equilibriation. In some embodiments, the cells from long term storage are shipped to the cell restoration site in a frozen state (e.g., in liquid nitrogen, dry ice, etc.). As shown in FIG. 5, in some embodiments, the cells are defrosted (either cells from long term storage or newly prepared). In some embodiments, for defrosting and equilibrating the cells, one or more of the following steps can be used.

In some embodiments, the equilibration of cells (e.g., patient or donor) includes one or more of the following steps and/or features. The equilibration of the patient cells is shown in FIGS. 5, 6, and 7. In some embodiments, patient cells or donor cells are thawed by placing them into a water bath (previously set to 37° C.) (e.g., for 5 minutes or until vials become visibly liquid). In some embodiments, take out plates from incubator and place under the hood. In some embodiments, add a volume of cells drop wise onto each dish. In some embodiments, gently place the plates with cells in the incubator. In some embodiments, allow the cells to warm for equal to or at least about: 1 hour, 2 hours, 3 hours, 4 hours, 5 hours, or ranges spanning and/or including the aforementioned values.

In some embodiments, after equilibration, the patient cells are removed from the incubator. In some embodiments, pipette up and down (cells+media from respective 100 mm plates). In some embodiments, spin down (at 300 g, room temperature, and for 10 minutes). In some embodiments, remove supernatant from cell pellets. In some embodiments, lightly tap and loosen pellets. In some embodiments, add Stem Span culture media and place back in incubator. In some embodiments, the donor cells are removed from the incubator. In some embodiments, pipette up and down (cells+media from respective 100 mm plates). In some embodiments, spin down (at 300 g, room temperature, and for 10 minutes). In some embodiments, remove supernatant until left with only cell pellets. In some embodiments, lightly tap and loosen pellets and then add MED-CR-100+(SS+) culture media and place back in incubator.

In some embodiments, both patient and donor cells are ready to be counted (e.g., with Turk's and Trypan blue). In some embodiments, label disposable culture tubes with respective sample name. In some embodiments, add Turk's/Trypan on each tube. In some embodiments, add cells and mix well. In some embodiments, place the remainder of the cells back into incubator. In some embodiments, proceed to counting. In some embodiments, after counting the cells, then proceed to seeding cells onto 6 well plates and transwell inserts based on desired seeding density (20-30×10⁶ cells per well).

Transwell Culture: As shown in FIGS. 3A.6, 5, 8, and 9, in some embodiments, a transwell culture is performed. In some embodiments, place patient cells in a transwell plate. In some embodiments, the patient cells are placed in the transwell plate by placing a volume of patient cells to create a cell suspension of about 20-30×10⁶ cells per inner well. In some embodiments, this cell suspension is supplemented with MED-CR-100+ to a final volume of 3.5 mL per inner well. In some embodiments, with sterile tweezers place transwell inserts into inner wells. In some embodiments, place a volume of donor cell suspension equivalent to same number of cells used above in the inner well per transwell insert. In some embodiments, supplement this cell suspension volume with restoration media to a final volume of 3.5 mL per transwell insert. In some embodiments, close all plate lids and place into incubator for culture.

In some embodiments, the media is supplemented during the transwell process. Day 3 Mid-Cultivation Media Supplementation: In some embodiments, after three days, place MED-CR-100+ bottle into water bath set to 37° C. for 30±2 minutes. Ethanol wipe and UV laminar flow hood for a minimum of 10 minutes and maximum of 12 minutes. After media is warmed to 37±1C, take out plates from incubator and place into laminar flow hood. Take 200 μL of cell suspension from an arbitrary well and place in sterile 2 mL microcentrifuge tube and place aside. Use this aliquot for in-process testing (SOP-TEST-001 (InvivoGen PlasmoTestMycoplasma Detection Kit), -002 (cell viability Trypan Blue test); Form-001, -002).

In some embodiments, without removing the transwell inserts from the plates, supplement each well and transwell insert with 1 mL of 37±1° C. MED-CR-100+. Visually check for any color change in the media, or any presence of contamination. If no contamination detected, place all plates back into incubator for the remainder of the culture.

In some embodiments, when the transwell process is done, acquire MED-CR-100+ at 37° C. In some embodiments, take out plates from incubator. In some embodiments, take 200 μL of cell suspension from an arbitrary well and place in sterile 2 mL microcentrifuge tube and place aside. In some embodiments, use this aliquot for in-process testing (SOP-TEST-001, -002; Form-001, -002) for sterility. In some embodiments, check for any color change in the media, or any presence of contamination. If no contamination detected, place all plates back into incubator for the remainder of the culture. After a minimum of 16 h and a maximum of 24 h, the results of the sterility test will be available. If results of sterility test are negative for contaminants, proceed to the next steps of the protocol.

In some embodiments, a wash/infusion buffer is prepared for final formulation. In some embodiments, prepare 1000 mL of wash/infusion buffer, herein referred to as MED-WI-100. In some embodiments, to make 1000 mL of wash/infusion buffer, herein referred to as MED-WI-100, combine normal saline and human serum albumin into each of 2×1 L sterile disposable bottle.

Processing, washing and final formulation of the cell product: The following describes procedures performed as shown in FIGS. 3A.7, 4.8, 5, and 8. In some embodiments, place MED-WI-100 bottle into water bath set to 37° C. for 30±2 minutes. In some embodiments, place a 500 mL of normal saline, 0.9% sodium chloride injection bag to pre-warm on an injection bag. In some embodiments, allow the buffer to warm to 37±1° C. In some embodiments, the well media/cells are collected. In some embodiments, the cells are spun down (e.g., at 300 g, room temperature, and for 10 minutes). In some embodiments, supernatant is removed until left with cell pellets.

In some embodiments, lightly tap and loosen pellets. In some embodiments, add wash buffer to each loose pellet. In some embodiments, gently pipette up and down to loosen pellet. In some embodiments, add 39 mL of MED-WI-100 (wash buffer). In some embodiments, spin down (e.g., at 300 g, room temperature, and for 10 minutes). In some embodiments, remove supernatant to provide cell pellets. In some embodiments, lightly tap and loosen pellets and add 5 mL of MED-WI-100 (wash buffer) to each loose pellet. Combine volume of each conical tube into 1×50 mL conical tube [20 mL total volume of cells/MED-WI-100 (Wash buffer)].

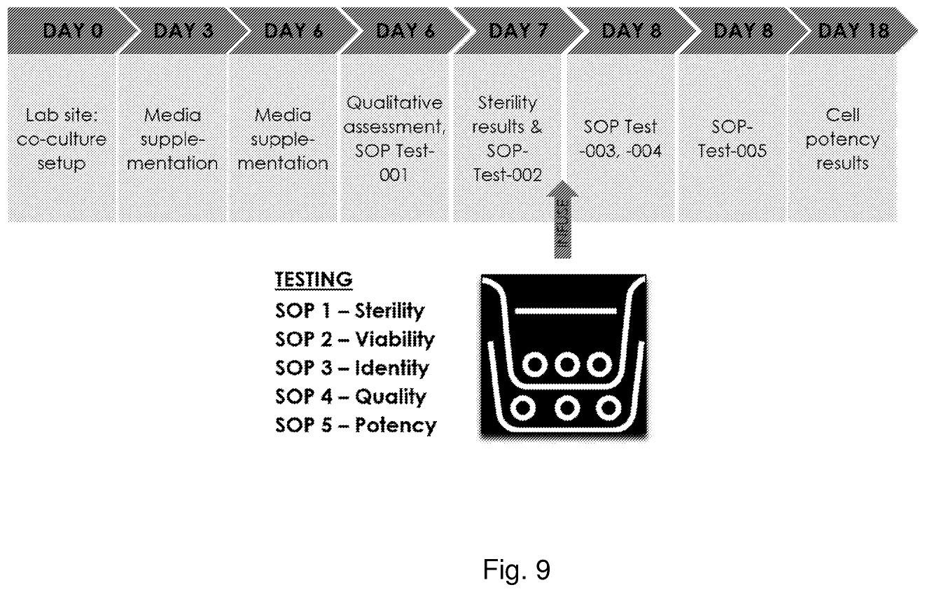

In some embodiments, as shown in FIG. 9, final in-process testing is performed (SOP-TEST-002, Form-002, -200). In some embodiments, if the minimum viability of 65% from SOP-TEST-002 is obtained, proceed to next step. If this minimum value is not obtained, refer to SOP-LAB-003 (SOP test for cell identity in mobilized peripheral blood through cell surface antibody staining and flow cytometry). Cell quality can be tested using a cell vitality assay (e.g., a two color fluorescence assay that distinguishes metabolically active cells from injured cells and dead cells). Cell potency can be tested using a clonogenic assay, which utilizes specialized growth media to test the number of colony-forming units (CFUs) within a culture containing hematopoietic stem and progenitor cells. A mobilized blood culture yielding large numbers of CFUs would be considered highly potent, while one yielding few or no CFUs would be considered minimally potent. The cell identity, quality, viability, vitality testing and other “SOP” testing can be performed at any time, including after harvesting of cell, after cryogenic treatment, after exposure to other cells using a transwell plate, prior to infusion, etc. FIGS. 9B-9D provide alternative embodiments for logistics in the clinical processing of cells.

Examine final formulated product for any evidence of cell aggregation or clumping. *Note: Evidence of aggregation or clumping during final filling may be grounds to discontinue the protocol if no resolution can be implemented. In some embodiments, this is important during the filling of the final cell suspension into syringes and infusion bag. In some embodiments, if cell aggregation or clumping is observed, cell suspensions can be passed through a sterile cell strainer (100 μm—Fisher 22363549) to clarify the product. In some embodiments, if aggregates remain, discuss with clinical staff the risk to patient or no further action. In some embodiments, if the clinical risk is low, proceed. At this point, cells are ready to be infused. Proceed to transferring final volume into infusion bags for patient infusion.

Preparation of the infusion bag: In some embodiments, at this time, note the total volume of cell product above and the Final Volume for Infusion and subtract to determine Volume of Normal Saline, 0.9% sodium chloride injection that should be added. In some embodiments, remove the pre-warmed normal saline, 0.9% sodium chloride, 500 mL injection bag. In some embodiments, clean with ethanol and place into hood and thoroughly clean the self-healing port with a 70% isopropanol cleaning wipe and allow 10 seconds to dry. In some embodiments, clean the self-healing port of an empty 250 mL Infusion bag with a 70% isopropanol cleaning wipe and allow 10 seconds to dry. In some embodiments, using a sterile 60 mL syringe and 18 G needle, withdraw a volume of saline (the Volume of Normal Saline above) from the pre-warmed 500 mL saline injection bag and load into the sterilized empty 250 mL bag. In some embodiments, invert empty 250 mL infusion bag and load pre-warmed saline through the self-healing port. In some embodiments, repeat with same syringe and needle until the desired volume has been achieved.

In some embodiments, prepare a sterile 60 mL syringe with a sterile 18 G needle and insert into the same self-healing port of the 250 mL infusion bag. In some embodiments, using a 5 or 10 mL pipette, back load the cell product (resuspended in MED-WI-100) into a sterile, un-plunged 60 mL syringe and allow the cell product to gravity flow into the 250 mL infusion bag. In some embodiments, repeat these steps until all of the cell product is injected. Note the Final concentration of HSA in saline bag after filling.

In some embodiments, remove the syringe and slowly rotate the 250 mL injection bag to mix the cell product with the saline. In some embodiments, observe if any clumping or aggregation occurs. If no clumping or aggregation is detected, the cell product is now ready to be transferred to the clinician.

Patient Infusion: As shown in FIG. 3A.9, the cells can then be infused with AR-100. In some embodiments, after mixing the content of the 250 mL saline bag, the bag is spiked on one spike of the blood tubing and the 500 mL bag of saline is spiked on the other. In some embodiments, the blood tubing is primed with the 500 mL bag of saline. In some embodiments, the Y tubing to the 500 mL saline bag is clamped once the tubing is primed. In some embodiments, the blood tubing is then connected to the lowest Y tubing site of the patient's IV tubing and the 250 mL bag of saline+cell product is infused over 60 to 90±2 minutes.

In some embodiments, once the 250 mL bag is empty, it is gravity fed by the 500 mL bag to clear the bag and tubing (flushing all cells out of the 250 mL bag and Y tubing); this is allowed to infuse into the patient. In some embodiments, once flushed and emptied the Y tubing to the 250 mL bag is clamped. In some embodiments, the Y tubing to the 500 mL bag is then opened and approximately 150 to 200 mL of pure saline is flushed to clear lines and make sure all cells are infused.

Make 10 mL of cryogenic media for PT-006 cells: In some embodiments, cryogenic media is prepared. In some embodiments, to make 10 mL of cryogenic media, herein defined by the designation MED-CRYO-100, add Dimethyl Sulfoxide (DMSO, Fisher Scientific, catalog #BP231-100) and HSA into a 50 mL conical tube. Place solution at 4° C. until ready to be used.

Freezing of PT-006 cells: In some embodiments, label cryogenic vials with cell type, patient number, number of cells, date and operator's initials. In some embodiments, place cryogenic freezing container in the fridge for 15 minutes. In some embodiments, spin down the 25-50×10⁶ PT-006 cells set aside above (e.g., at 300 g, room temperature, and for 10 minutes). In some embodiments, take out cryogenic freezing container and MED-CRYO-100 media from the fridge, spray with 70% ethanol and place under the sterile hood. In some embodiments, remove supernatant from conical tube until left with only cell pellet. In some embodiments, lightly tap conical tube to loosen pellet, and then pipette MED-CRYO-100 media to resuspend cells; and immediately pipette into labeled cryogenic vial, for a final concentration of approximately 25-50×10⁶ cells/mL. In some embodiments, place the cryogenic vial in the cryogenic freezing container and place immediately at −80° C.

### Embodiments of Kits for Clinical Processing of Cells

Some embodiments disclosed herein pertain to kits providing instructions, containers, compositions, and methods used as part of a process for improving and/or restoring one or more cellular functions to cells. In some embodiments, cellular function is improved in a target cell (or target cells). In some embodiments, the target cell can be introduced to a patient to achieve one or more beneficial effects in a patient. In some embodiments, disclosed herein are kits that provide protocols for testing patients and donors. In some embodiments, disclosed herein are kits that provide protocols to determine whether patients or donors can be used in the disclosed methods of treatment.

In some embodiments, the kits provide instructions, methods, and equipment to improve the quantity and quality of target cells. In some embodiments, these kits include instructions and equipment for methods of isolating patient and/or donor cells, methods of preparing target cells using patient and/or donor cells, methods or instructions for transporting cells, methods or instructions for storing cells, and the like. In some embodiments, the kits provide instructions, methods, and equipment for one or more of the following: autologous and/or allogeneic treatments; co-culture-based restoration that requires no genetic manipulation of cells; isolating freshly collected cells (e.g., donor cells, patient cells, or target cells that are produced and injected into patients) or for cells that have been stored for years (e.g., patient, donor, or target cells that have been stored for a period of years that is greater than or equal to: 1, 2, 3, 5, 10, 15, or ranges including and/or spanning the aforementioned values); cells with improved viability; cells with improved quality. While several embodiments make reference to testing kits for patients, the testing kits for donors can comprise (or lack) one or more or all of the components provided in patient testing kits.

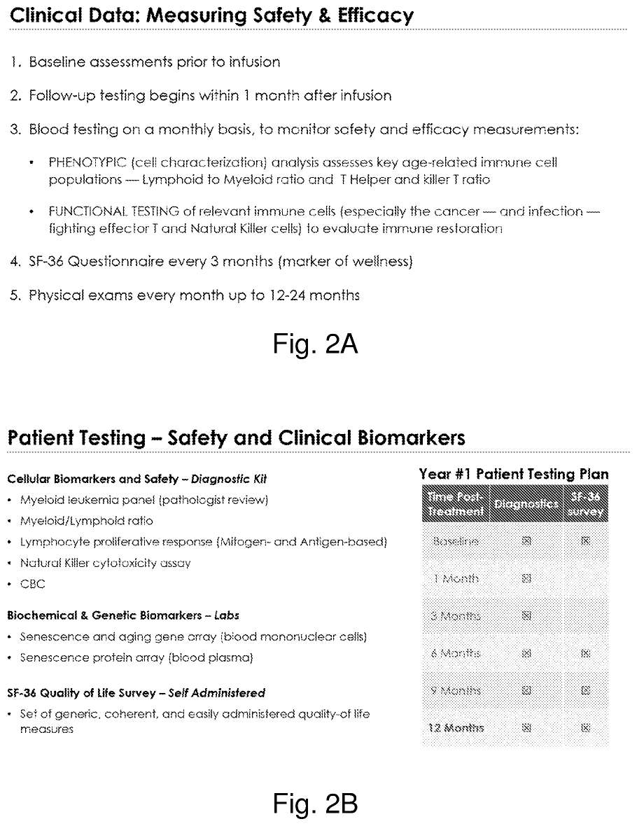

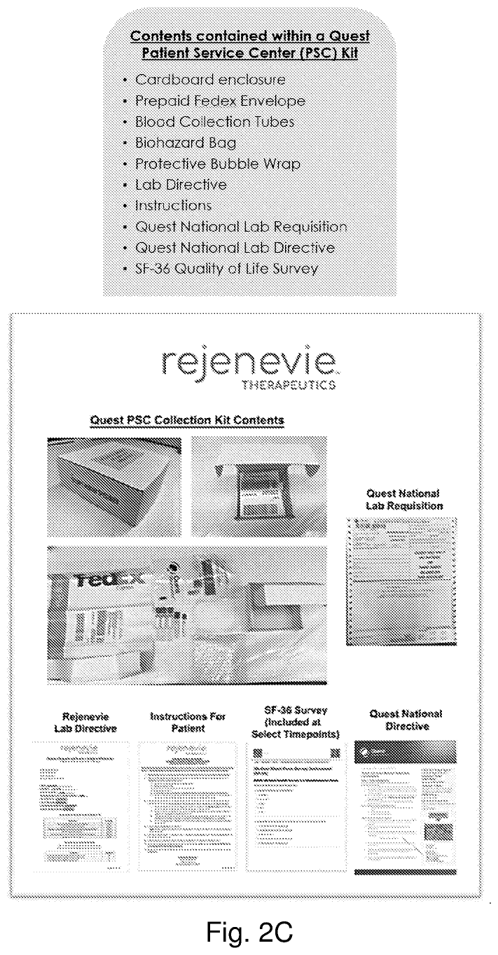

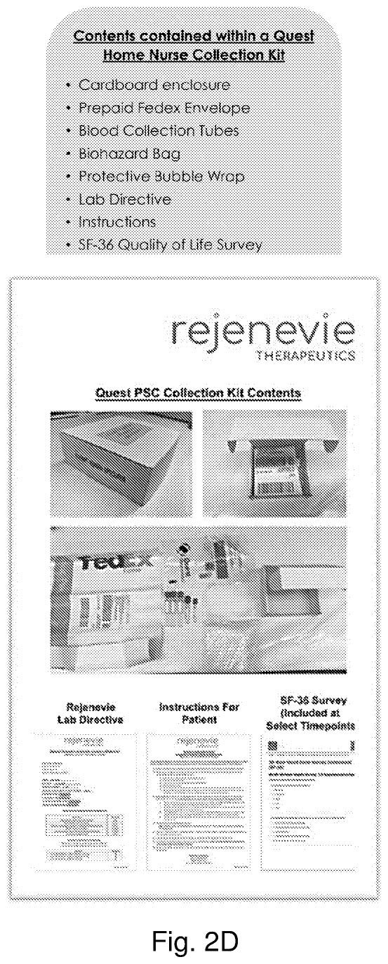

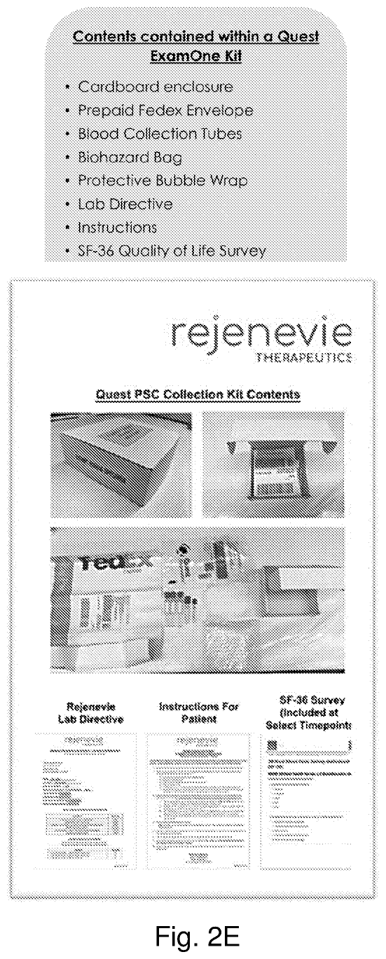

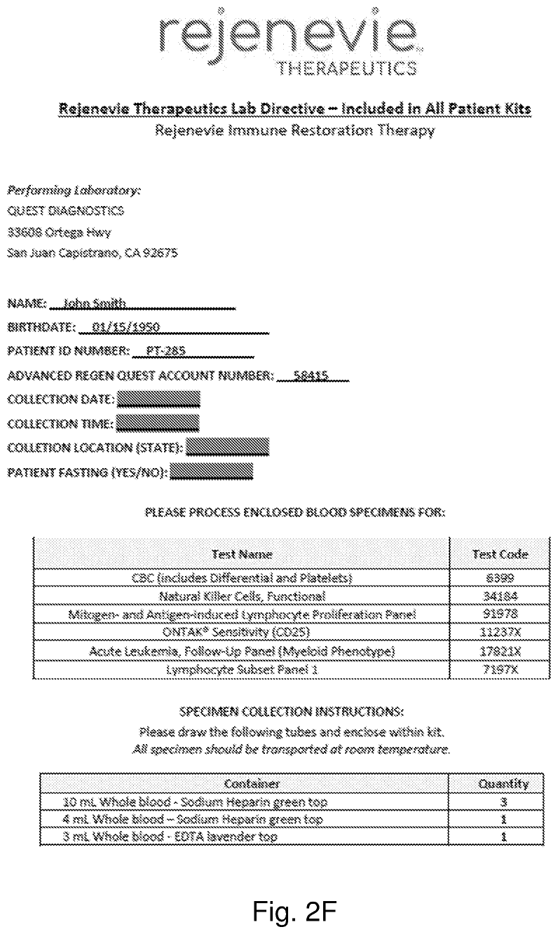

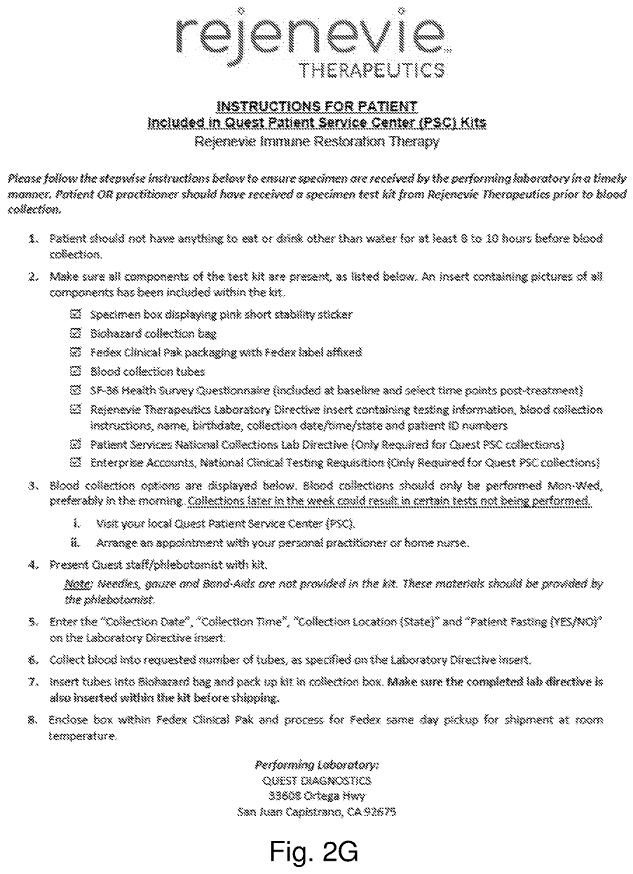

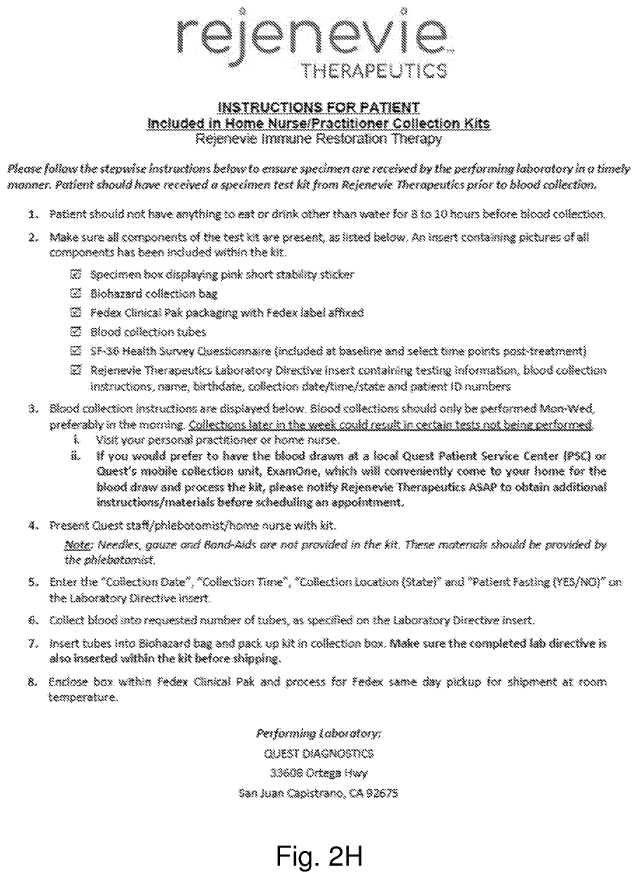

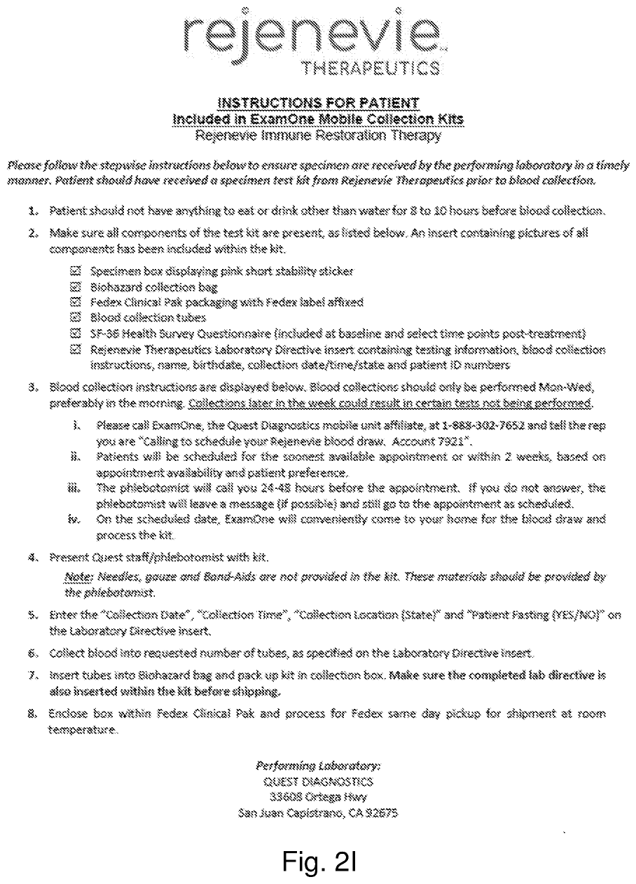

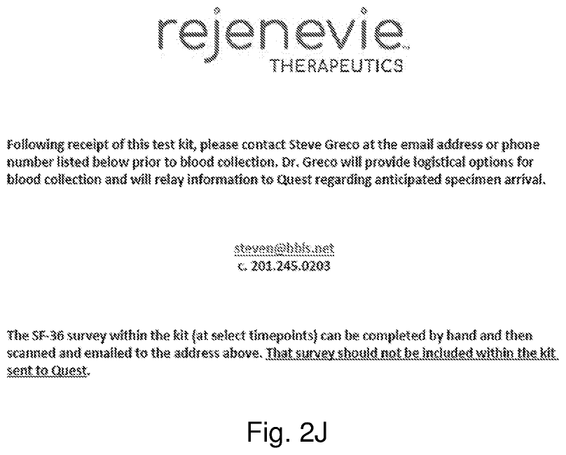

FIG. 2A shows a flow chart for collecting clinical data for measuring the safety and efficacy of methods disclosed herein. In some embodiments, a baseline assessment is performed on a patient prior to treatment with a method disclosed herein. As disclosed elsewhere herein, the baseline testing can include questionaires, blood testing, physical examinations, and combinations thereof. In some embodiments, the kits provided herein provide instructions, containers, compositions, and methods used as part of a process for making baseline assessments of the patient prior to treatment. In some embodiments, the kits provided herein provide instructions on when and how to carry out patient assessments (baseline and after treatment). In some embodiments, following baseline assessment, a selection step is performed to determine if the patient would benefit from treatment. Where the patient is a good candidate for treatment, the patient is selected for infusion with target cells (as disclosed elsewhere herein).

In some embodiments, as shown, after a treatment session is performed (e.g., administration of a target cell, RNAi, or small molecule as disclosed herein), follow-up testing can be performed. In some embodiments, this follow-up testing can be used to determine if the patient is a candidate for further treatment. In some embodiments, after infusion, follow-up testing is performed within less than or equal to 1 month, 2 months, 6 months, or ranges including and/or spanning the aforementioned values. In some embodiments, as shown in FIG. 2A, the testing may be performed on a monthly basis. In some embodiments, the testing can be used to assess the safety and/or efficacy of ongoing treatment and/or of the initial treatment. In some embodiments, as disclosed elsewhere herein, the patient assessment testing can include questionaires, blood testing, physical examinations, and combinations thereof. In some embodiments, as disclosed elsewhere herein, periodically (e.g., after testing), selection determinations are made to determine whether the patient should be treated again or should be withdrawn from treatment. Such assessments may be made at periods after treatment of less than or equal to 1 month, 2 months, 6 months, 12 months, 18 months, 24 months, or ranges including and/or spanning the aforementioned values.

In some embodiments, as disclosed elsewhere herein, patient testing can include blood testing. In some embodiments, kits are provided to users so that specific testing panels can be performed to assess the safety and/or efficacy of treatment. In some embodiments, the kits include instructions for specific testing and/or equipment (e.g., vials, etc.) for such testing. In some embodiments, the assessment includes phenotypic testing (e.g., cell characterization) and determines the presence or prevalence of certain age-related immune populations and their ratios (e.g., lymphoid to myeloid ratios; T-helper cell to killer T cell ratios; etc.). In some embodiments, the lymphoid:myeloid ratio is less than or equal to 1:1, 1:2, 1:5, 1:10, or ranges spanning and/or including the aforementioned values. In some embodiments, the T helper: Killer T ratio is less than or equal to 1:1, 1:2, 1:5, 1:10, or ranges spanning and/or including the aforementioned values. In some embodiments, based on phenotypic test results, patients can be withdrawn from treatment or further treated. In some embodiments, as shown, functional testing can be performed. In some embodiments, the number or amount of relevant immune cells can be measured. In some embodiments, those immune cells may include cancer cells, cells that are indicative of infection, and/or other cells that are indicative of positive or negative clinical outcomes. In some embodiments, the immune cells include fighting effector T-cells and natural killer T cells. In some embodiments, the presence or absence of these cells allows determination of whether immune restoration by treatments as disclosed herein are successful and whether the patient is a candidate for further treatment or withdrawal from treatment. In some embodiments, based on patient assessment results as disclosed herein (e.g., blood testing, questionaires, physicals, other tests, and combinations thereof), patients' treatment can be accelerated (with more frequent infusions of target cells) or slowed (with less frequent infusions) based on test results.

In some embodiments, the kits may include questionaires that determine whether a patient is responding positively or negatively to treatment. In some embodiments, the method of treatment as disclosed elsewhere herein may include the administration of a questionnaire to the patient. In some embodiments, the questionnaire is a SF-36 questionaire or the like. In some embodiments, the questionnaire asks one or more of testing panels can be performed to assess the safety and/or efficacy of treatment. In some embodiments, the list of questions in the questionnaire include patient-reported outcomes (PROs) usually labeled quality of life (QoL) measures. In some embodiments, after infusion, the kit includes instructions for the patient to complete a questionnaire every 2 months, 3 months, 6 months, or ranges including and/or spanning the aforementioned values. In some embodiments, the kit includes instructions on where to send the completed questionaires after completely. In some embodiments, a baseline assessment is performed prior treatment (e.g., prior to administration of a target cell, RNAi, or small molecule) or on the first day of treatment. In some embodiments, after treatment, the method of treating the patient may include administration of the questionnaire every 2 months, 3 months, 6 months, or ranges including and/or spanning the aforementioned values. The kit may include instructions regarding the frequency of these assessments.

In some embodiments, the kits may include instructions indicating that physical exams should be performed on the patient periodically. In some embodiments, the methods of treatment may include periodically administering physical exams on the patient. In some embodiments, these physical exams may test indicators of health and efficacy of treatment to determine whether a patient is responding positively or negatively to treatment. In some embodiments, after treatment (e.g., administration of a target cell, RNAi, or small molecule), the method of treatment includes (and/or the kit includes instructions for) performing physical examinations on the patient at time periods of equal to or less than every 6 months, 12 months, 18 months, 24 months, or ranges including and/or spanning the aforementioned values. In some embodiments, a baseline assessment is performed prior treatment (e.g., prior to administration of a target cell, RNAi, or small molecule) or on the first day of treatment.

FIG. 2B provides a regimen of testing that may be performed in the methods of treatment as disclosed herein. In some embodiments, the kits disclosed herein include a testing schedule with time points for testing and instructions therefor as shown in FIG. 2B. In some embodiments, as shown, the schedule may include testing for safety and clinical biomarkers. In some embodiments, the kit may include instructions and/or equipment for (or the method may include steps for) testing cellular biomarkers and safety (e.g., Quest Diagnostic Kit). In some embodiments, those tests may include one or more of myeloid leukemia panel (pathologist review), myeloid/lymphoid ratio, lymphocyte proliferative response (Mitogen- and Antigen-based), natural killer cytotoxicity assay, and/or CBC testing. In some embodiments, the kit may include instructions and/or equipment for (or the method may include steps for) testing biochemical and/or genetic biomarkers, including one or more of senescence and aging gene array (blood mononuclear cells), senescence protein array (blood plasma), SF-36 Quality of Life Survey (e.g., self administered), a set of generic, coherent, and easily administered quality-of life measures. In some embodiments, such assessments (or instructions for such assessments) may be made at periods after treatment of less than or equal to 1 month, 2 months, 3 months, 6 months, 12 months, 18 months, 24 months, or ranges including and/or spanning the aforementioned values.

As shown in FIGS. 2C-2E, some embodiments pertain to a kit for collecting blood from a patient. In some embodiments, as shown, the kit comprises liquid collection containers. In some embodiments, the liquid collection containers are configured to receive blood. In some embodiments, the liquid collection containers are blood collection tubes or vials. In some embodiments, the kit comprises a laboratory directive. In some embodiments, the laboratory directive provides instructions to nurses, technicians, or doctors on what blood tubes to fill, what tests to perform, where to ship contents, and the like. FIG. 2F provides examples of laboratory directives. In some embodiments, as shown in FIG. 2C, the kit comprises a Quest lab requisition and lab directive. In some embodiments, this kit can be used where the patient is tested at a Quest service center. In some embodiments, as shown in FIGS. 2D-2E, the kit can comprise a laboratory directive for nurses or ExamOne personnel. In some embodiments, the lab directive provides instructions for testing the blood samples. In some embodiments, the kit comprises a lab requisition form. In some embodiments, the lab requisition form is for a national laboratory. In some embodiments, the lab requisition form is a Quest National Lab Requisition form. In some embodiments, the kit comprises a diagnostic testing unit or a directive to perform the testing disclosed on the testing unit. In some embodiments, the diagnostic testing unit comprises a diagnostic testing kit comprising one or more of a myeloid leukemia panel, a myeloid/lymphoid ratio assay, a lymphocyte proliferative response assay, a natural killer cytotoxicity assay, a T helper cell/killer T cell ratio assay, and/or a complete blood count assay. In some embodiments, the lymphocyte proliferative response assay is mitogen-based and/or antigen-based. In some embodiments, the diagnostic testing unit comprises biochemical and/or genetic biomarker assays. In some embodiments, the diagnostic testing unit comprises one or more of a senescence gene array, an aging gene array, and/or a senescence protein array. In some embodiments, the kit comprises a senescence gene array and/or aging gene array measure blood is configured to measure mononuclear cells. In some embodiments, the senescence protein array is configured to measure blood plasma proteins.

In some embodiments, the kit allows on time processing of samples. In some embodiments, timing is important because several of the tests are time sensitive. In some embodiments, certain tests on the panel must be in process within 48 h of blood collection and others in less than 72 h. In some embodiments, since our patients are being collected all over the country and/or world (e.g., more than 500 miles, 1000 miles, 1500 miles away from blood processing centers), specific and clear instructions on how to handle, process and transport the blood once collected may be provided. In some embodiments, failure to follow these instructions could lead to samples “timing-out” and not being run, which causes inconvenience and additional testing (which may also time out). In some embodiments, proper execution of specimen being tested allows the evaluation of the safety and efficacy of a particular immune restoration technology.

In some embodiments, the kit comprises patient instructions (as shown in FIG. 2G-2J). Examples of patient instructions, ExamOne technician instructions, or Quest technician instructions and the contents thereof are provided in FIGS. 2G-2J. Patient instructions may include one or more of the following instructions: that the patient should not have anything to eat or drink other than water for at least 8 to 10 hours before blood collection; to make sure all components of the test kit are present, as listed below (an insert containing pictures of all components may be provided showing one or more of a specimen box displaying pink short stability sticker; a biohazard collection bag; a Fedex Clinical Pak packaging with Fedex label affixed; a Blood collection tubes; a SF-36 Health Survey Questionnaire (included at baseline and select time points post-treatment); a Rejenevie Therapeutics Laboratory Directive insert containing testing information, blood collection instructions, name, birthdate, collection date/time/state and patient ID numbers; a Patient Services National Collections Lab Directive (Only Required for Quest PSC collections); an Enterprise Accounts, National Clinical Testing Requisition (Only Required for Quest PSC collections). In some embodiments, the instructions provide blood collection options. In some embodiments, blood collections instructions request blood collections on Monday, Tuesday, or Wednesday Mon-Wed (e.g., in the morning) because collections later in the week could result in certain tests not being performed (e.g., because facilities are not available).

In some embodiments, the instructions provide that the patient should do one or more of the following: i. Visit your local Quest Patient Service Center (PSC) ii. Arrange an appointment with your personal practitioner or home nurse. In some embodiments, the instructions provide that the patient should present Quest staff/phlebotomist with kit. In some embodiments, the logistics around specimen collection with 3 convenient options: home nurse, Quest PSC or ExamOne mobile service. In some embodiments, this is a unique offering with stringent logistics established to get samples to the Quest lab in California before any tests “time-out” (e.g., is expired due to the blood sample degrading or becoming inaccurate). In some embodiments, a successfully completed test panel will yield results that suggest how competent a patient's immune system is.

In some embodiments, the cardboard enclosure containing short stability indicator (e.g., a pink short stability sticker provided by Quest as shown in the kit pictures). In some embodiments, the sticker ensures timely processing of specimens by laboratory facilities. In some embodiments, the sticker is what signals the lab (in California for Quest) to move the specimen along in a timely fashion for processing. In some embodiments, previous experiences with this test panel before the kit would led to degradation or false readings from the kit and delayed processing. In some embodiments, without such labeling, samples would not be processed within the 48 h window for some of these tests (e.g., some tests require processing within 48 hours of blood drawing or test results could be inaccurate). In some embodiments, the enclosure and sticker avoid that problem or others by helping ensure timely processing. In some embodiments, some of these tests are only feasible as a national lab making the indicator a useful option for on time examination of test samples.

In some embodiments, the instructions provide that needles, gauze and Band-Aids are not provided in the kit. These materials should be provided by the phlebotomist. In other embodiments, needles, gauze and Band-Aids are provided in the kit. In some embodiments, the instructions provide that the patient or blood drawer should enter one or more of the “Collection Date”, “Collection Time”, “Collection Location (State)” and “Patient Fasting (YES/NO)” on the Laboratory Directive insert. In some embodiments, the instructions provide that blood should be collected into requested number of tubes, as specified on the Laboratory Directive insert. In some embodiments, the instructions provide tubes should be inserted into Biohazard bag and pack up kit in collection box. In some embodiments, the instructions provide that the packager should ensure the completed lab directive is also inserted within the kit before shipping. In some embodiments, the instructions provide that the patient or blood drawer should enclose the box within Fedex Clinical Pak and process for Fedex same day pickup for shipment at room temperature.

In some embodiments, the blood collection kit is configured for immune restoration patients. In some embodiments, a national lab (e.g., Quest diagnostics) may provide one or more of the outer cardboard housing with pink short stability sticker, biohazard bag, Fedex prepaid shipper and bubblewrap (as shown). In some embodiments, also provided may be one or more of the blood collection tubes, lab directive, kit instructions, kit picture document (e.g., showing contents), and general health status questionnaire (e.g., SF-36). In some embodiments, a national lab provides the national lab requisition for patients opting to have their blood drawn at one of the nationwide locations patient service centers (PSCs) (e.g., Quest). For patients being collected at a PSC, an additional national lab directive (e.g., Quest) is also provided in the kit (as shown).

In some embodiments, an advantage is that a national lab (e.g., such as Quest) may be one of the few, if not only, laboratories in the US that is able to perform the function NK assay and T cell proliferation assays (or other tests). In some embodiments, the laboratory is CLIA certified, etc. In some embodiments, to obtain patient data from a clinical lab for these complex and predictive tests of a patient's immune function, is significant. In some embodiments, there is no other blood collection kit that is used to perform these sophisticated tests in a clinical lab.

In some embodiments, the kit comprises an enclosing container configured to house other components of the kit. In some embodiments, the kit comprises a shipping envelope configured to receive samples prepared using the kit. In some embodiments, the shipping envelope is prepaid. In some embodiments, the shipping envelope provides for overnight shipping.

In some embodiments, the kit comprises a patient self-evaluation form. In some embodiments, the self-evaluation form is a quality of life form. In some embodiments, the self-evaluation is a questionnaire providing simple questions and multiple choice optional answers (e.g., with three or five options). In some embodiments, the questionnaire requests the patient's perceived health status (e.g., is your health: A) poor, B) fair, C) good, D) very good, E) excellent), health over a period of time (e.g., has your health: A) declined relative to a year ago, B) somewhat declined relative to a year ago, C) about the same as a year ago, D) somewhat improved relative to a year ago, E) improved relative to a year ago), activity levels and examples (do you limit your activity in any of the following areas: vigorous exercise (as running, lifting heavy objects, participating in strenuous sports); moderate exercise (moving furniture, playing golf); Light exercise (moving groceries from the car to the house, climbing stairs, bending, kneeling, walking, bathing) answered with “unable”, “somewhat able”, “able but limited”, or “able”), how much pain the patient is in, how much the pain interfered with activity level, whether the patient limited their activities and how much, and the like. In some embodiments, the self-evaluation form is a SF-36 quality of life survey or the like.

In some embodiments, the kit comprises a biohazard container. In some embodiments, the biohazard container is a bag. In some embodiments, the laboratory directive comprises blood drawing instructions.

In some embodiments, the kit comprises instructions indicating that the diagnostic testing should be performed about every month. In some embodiments, the kit comprises instructions indicating that a physical examination of the patient should be performed about every 12 to 24 months. In some embodiments, the patient instructions and/or the self-evaluation form indicates that it should be completed about every three months. In some embodiments, the kit comprises instructions indicating that a baseline physical examination and diagnostic testing should be performed prior to treatment.

In some embodiments, the kit comprises a packing material. In some embodiments, the packing material is bubble wrap.

In some embodiments, the kits provide methods, instructions, or equipment for health screening on a would-be patient. In some embodiments, the kits provide methods, instructions, or equipment for health screening on a would-be donor. In some embodiments, based on the health screening the would-be patient and/or the would-be donor either may be excluded or included from additional steps in the method. In some embodiments, the donor may be the patient at a younger age.

In some embodiments, the kits may be used to deliver cells to one or more locations disclosed in FIG. 3A, including the leukopheresis facility, the long term storage facility, the restoration facility, the patient infusion facility, or the follow-up testing facility. In some embodiments, the kit provides instructions and equipment for such shipment. In some embodiments, the kit provides logistics established to support testing of patients and donors across a country (e.g., the US). In some embodiments, the kit provides additional test panels. In some embodiments, the kit provides these panels could be related to the current treatment (immune function), or could be expanded to include other tests of physiologic function associated with aging (cardiovascular, metabolic, etc.).

Kits may include package(s) or containers comprising the compositions disclosed herein (e.g., RC, cell-free culture media) and may include defined culture medium and cell culture medium supplement. The kit may further include an instruction letter or package-associated instruction for the treatment and/or prophylaxis of a medical condition. The phrase “package” means any vessel containing the compositions (including stem cells, media, and/or media supplement) presented herein. For example, the package can be a box or wrapping. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. The kit can also contain items that are not contained within the package but are attached to the outside of the package, for example, pipettes. Kits may optionally contain instructions for administering compositions of the present disclosure to a subject having a condition in need of treatment. Kits may also comprise instructions for approved uses of compounds herein by regulatory agencies, such as the United States Food and Drug Administration. Kits may optionally contain labeling or product inserts for the present compositions. The package(s) and/or any product insert(s) may themselves be approved by regulatory agencies. The kits can include compounds in the solid phase or in a liquid phase (such as buffers provided) in a package. The kits also can include buffers for preparing solutions for conducting the methods, and pipettes for transferring liquids from one container to another. The kit may optionally also contain one or more other compounds for use in combination therapies as described herein. In certain embodiments, the package(s) is a container for intravenous administration.

According to another aspect of the disclosure, kits may provide therapeutics. As disclosed elsewhere herein, in some embodiments, the kits may be configured to allow transport and/or collect cells (including donor cells, patient cells, target cells, and combinations thereof). In some embodiments, the kits contain instructions and equipment for the collection and shipment of donor cells, patient cells, target cells, and combinations thereof. In some embodiments, the kits may further contain agents used to produce target cells, including but not limited to RNAis, miRNAs, siRNAs, and small molecules for treating patient cells.

In some embodiments, the kit can be taken by the patient to a blood drawing center. In some embodiments, the kit may be stored at a blood drawing center. In some embodiments, the blood drawing center or off-site technician (e.g., at-home nurse) performs one or more of the following services: inbound call center for initiating collection process, scheduling of specimen collection, Inventory and store collection kits and appropriate supplies, distribute collection kits using account (e.g., FedEx, or similar), reminder calls prior to date and time of collection appointment, in-home mobile phlebotomist to perform specimen collection, proper specimen collection, handling, and preparation for shipment, overnight collected specimen via Customer's FedEx account to appropriate Customer laboratory for testing, provide a standard reporting package on a regular basis detailing the program's operational performance and status, including call activity, scheduling, and completed visits. In some embodiments, the blood drawing center or off-site technician (e.g., at-home nurse) performs one or more of the following services: the drawing of blood or collection of urine specimens, including routine blood drawing and venipuncture techniques, butterfly/syringe techniques, finger sticks and other techniques; collecting physician, patient, and payer information to allow for proper billing and/or reporting of results; processing specimens, including pipetting, centrifuging, transferring serum or plasma into other tubes, freezing, preserving, dipping and slide preparation; packaging specimens for pick-up and delivery for laboratory testing; labeling specimens as required and placing all necessary items in the specimen envelope; and processing other routine paperwork generally associated with specimen collection. In some embodiments, the patient, blood drawing center, or off-site technician (e.g., at-home nurse) performs one or more of the following services: provide, directly or through other providers, medical authority and oversight on all lab orders, review of results, and participant notification of adverse results, by credentialed physician licensed in the state in which services are performed; provide, directly or through third-parties, approval of any call scripting associated with inbound or outbound call, provide, directly or through third-parties, the patient collection kit, including instructions and airbills, provide, directly or through third-parties, detailed instructions for phlebotomists to ensure proper specimen collection and handling. In some embodiments, patients participate in scheduled meetings to review program status and performance, and provide direction and guidance to the program as needed.

Some embodiments disclosed herein pertain to methods of preparing target cells and treating patients with the same. In some embodiments, one or more process steps disclosed herein for the preparation of target cells provide surprisingly increased viability and/or yield of the target cells. In some embodiments, by preparing cells using one or more techniques disclosed herein, improved patient outcomes (including increased vitality of target cells, etc.) can be achieved. In some embodiments, because diagnostic facilities, clinical facilities, cell laboratories, biorepositories, and processing laboratories may be in different locations (including at different facilities in different states or even in different countries), one or more of the disclosed methods can be used to improve testing, storage, and treatment outcomes. In some embodiments, disclosed herein are transport methods for patient, donor, and target cells that achieve increased viability and quality of such cells. Some embodiments disclosed herein pertain further to methods of using target cells for treating patients in need of treatment. In some embodiments, the methods of treatment include an administration of target cells or materials isolated to a patient suffering from, for example, an age-related disease, cancer, an infectious disease, or the like.

In some embodiments, once an acceptable patient and/or donor is found (e.g., using a kit as disclosed herein), one or more of the following steps is performed. In some embodiments, cells in the patient and/or donor are mobilized for a period of time (FIG. 3A.a1). In some embodiments, after mobilization, blood is collected from the patient and/or donor. In some embodiments, the mobilized patient and/or donor cells are collected (e.g., with leukapheresis) as a leukopak. In some embodiments, during leukopak collection, plasma and blood cells in collected from the blood are reduced to provide the leukopak. In some embodiments, the leukopak is packaged and shipped (at reduced temperature of less than or equal to about: 10° C., 8° C., 6° C., 4° C., 2° C., 0° C., or ranges spanning and/or including the aforementioned values) to a processing facility. In some embodiments, plasma is removed from the mobilized cells (e.g., greater than or equal to about: 25%, 50%, 75%, or ranges spanning and/or including the aforementioned values). In some embodiments, the cells are collected from the leukopak and mixed in a cryogenic medium. In some embodiments, the patient and/or donor cells are stored as a frozen mixture for a period of time. In some embodiments, the patient and/or donor cells are transported to a laboratory facility where the transwell process disclosed elsewhere herein is performed.

In some embodiments, provided herein is a clinical-grade culture protocol that utilizes, for example, mobilized cells, blood stem cells, and/or immune cells collected from donors (e.g., healthy young donors) to restore function to mobilized cells, blood stem, and/or immune cells collected from patients (e.g., healthy aged patients) using a transwell culture. In some embodiments, a transwell culture apparatus allows factors released from the donor cells to permeate the transwell membrane and interact with the patient cells (e.g., located in the bottom chamber). In some embodiments, exposure of the patient cells to the donor milieu is performed for a period of time (e.g., a number of days that is less than or equal to 5, 6, 7, 8, 10, or ranges including and/or spanning the aforementioned values). In some embodiments, the exposure increases stem cell and immune function in the patient cells, which can then be washed and prepared for infusion as an autologous cell therapy for the patient (e.g., back into the patient). In some embodiments, the methods provided herein can be performed using only human compatible media and/or media with only human-derived factors and reagents which can include, for example, human-based serum.

Prior to the embodiments disclosed herein, there were no available human protocols utilizing factors produced by young healthy blood stem cells and immune cells to restore function to the aging blood and immune systems. While research-grade protocols attempted to restore the function of aging stem cells, disclosed herein is are embodiments that utilize an autologous therapy that is clinical-grade. In some embodiments, cGMP principles are disclosed herein to generate donor, patient, and/or target cells as disclosed herein. In some embodiments, the protocol is devoid of and/or lacks any animal-derived products or supplements in the culture/restoration media. There are no comparative technologies that utilize young factors to restore function to the aging immune system as an autologous cell therapy for health aged patients. While some entities have transfused young plasma infusions into aged patients, this has now been outlawed in the US and lacked a scientific rationale for providing therapeutic benefit. This approach also lacked supportive preclinical data and carries unnecessary risk to the patient as this was a “non-self” therapeutic being infused into them (and has the capacity to transfer pathogens).

In some embodiments, the techniques for restoration and equilibration disclosed herein (and the “restoration media” as well as the “equilibration media”) yield safe and effective therapeutic protocols. In some embodiments, the protocol is devoid of any animal-derived products. Thus, in some embodiments, both the restoration media and equilibration media or xeno-free and considered clinical-grade. A significant hurdle in translating these types of media formulations from preclinical to clinical is being able to reproduce efficacy when removing the classic media growth factors supplied by the use of fetal bovine serum (FBS). In some embodiments, the restoration protocol disclosed herein has replaced FBS with human serum albumin that is commercially available, clinical-grade and cGMP. In some embodiments, a number of other “animal” or “xeno” free culture media for growing human cells can be used. In some embodiments, the media comprises a base available from Stem Cell Technologies (StemSpan) and is further supplemented. Available animal-devoid medias have never been used in a clinical manufacturing process similar to that disclosed herein.

In some embodiments, the restoration media is used during the transwell process (which may be a 7 day protocol or otherwise as disclosed herein). In some embodiments, the medium is used for producing the restored composition. In some embodiments, animal-devoid medium for blood and immune cells can be supplemented.

In some embodiments, frozen mobilized peripheral blood cells contains a complex mixture of immune cell types. Of note, neutrophils are notorious for not surviving the freeze-thaw process and lyse upon thawing. This lysis results in the release of DNA, which could easily cause the accumulation of viable cells to build up (clumping) and significantly reduce post-thaw yields (due to risks associated with clumping, such as stroke, etc.). In some embodiments, agents to prevent clumping are added to the equilibration medium. In some embodiments, the thawed vials of cells are dropwise added into the equilibration medium.

In some embodiments, cells are allowed to equilibrate in this equilibration media (e.g., at 37° C.) prior to addition to the transwell cultures. In some embodiments, the restoration media and equilibration media are used in other steps. In some embodiments, these could be used for second generation cell restoration approaches that do not utilize young cells, rather just the young factors that have been identified to be the mechanism of action (e.g., for micro RNAs and/or small molecules as disclosed elsewhere herein), in particular a combination of microRNAs. In some embodiments, the same equilibration and restoration medias would be utilized but for a gene therapy approach. In some embodiments, these media used for “scale-up” approaches where large numbers of transwell cultures would no longer be needed. In some embodiments, an apparatus would be similar to a single-use bioreactor. In some embodiments, these formulations could be utilized to restore the function of other aged non-immune cells and tissues, such as adipose-derived stem cells or stromal vascular fraction.

In some embodiments, the promotion of cellular health, as accomplished by the methods disclosed herein, may refer to alterations in parameters of cellular function that result in a perceived and/or quantifiable improvement in the viability state of cells and/or cell types. The viability state of a cell may be assessed using any suitable metric to evaluate parameters such as, but not limited to, cellular architecture, membrane organization and/or integrity, dynamic protein assemblies, molecular organization, and cellular responses to external signals. In some embodiments, the compositions and methods disclosed herein may improve the viability state of a cell as assessed by any suitable methodology. In some embodiments, a subject having improved and/or restored cellular function via the compositions and/or methodologies disclosed herein exhibits a perceived and/or quantifiable improvement in one or more aspects of the subject's cellular and/or general health. In some embodiments, the efficacy of the process can be measured through simple periodic blood draws post-treatment. In some embodiments, the processes disclosed herein can be used to treat one or more diseases linked to aging, including cancer, heart disease, stroke, Alzheimer's disease, and others. In some embodiments, the processes disclosed herein provide an immune restoration protocol for aging individuals to combat cancer and other age-related diseases.

In some embodiments, disclosed herein are methods comprising one or more steps of the following steps. In some embodiments, the method includes a step of obtaining a first cell sample from a first subject. In some embodiments, the method includes a step of obtaining a second cell sample from a second subject. In some embodiments, the method includes a step of culturing the first cell sample in culture media for a time period to produce a restoring medium. In some embodiments, the method includes a step of contacting the restoring medium with the second cell sample for a period of time to produce a target cell. In some embodiments, the method includes a step of administering the target cell to a patient in need of treatment.

### Logistics of Clinical Program

In some embodiments, the stepwise process disclosed in FIG. 3A is unique. In some embodiments, this process enables the safe and efficient transport of the freshly collected cells, processing and cryopreservation, biobanking, treatment (immune restoration) and safety/efficacy testing. In some embodiments, the process complies with cGMP standards mandated by the FDA. In some embodiments, the described clinical program logistics addresses a central problem in the cell therapy space: How to safely and efficiently collect, transport, store and treat patients with autologous therapies when there are geographical limitations. In some embodiments, using the methods disclosed herein, a patient can have a health screen, as well their stem cells mobilized and collected near most major cities within the US. In some embodiments, the process enables validated transport of cells from geographically vast collection sites to a centralized site for cell processing and cryopreservation short-term, with secondary transport to a biorepository for long-term storage. In some embodiments, the process enables transport of the cryogenically stored vials for shipment (e.g., international) to a clinical site (e.g., in the Bahamas) for clinical manufacturing and patient treatment. In some embodiments, the process has a established a testing regimen (e.g., with Quest diagnostics) for determining the safety and efficacy of the therapy. In some embodiments, this enables patient collections to occur anywhere in the US, even in the patient's own home. In some embodiments, the whole process in total is one of a kind, tailored to the patient and utilizes FDA guidance as set forth in the code of federal regulations toward the cell therapy industry as the standard.

In some embodiments, as disclosed elsewhere herein, the process spans a patient being screened and deemed healthy enough for stem cell mobilization to receiving the autologous immune restoration therapy at the clinic. In some embodiments, as disclosed elsewhere herein, patients have specific inclusion and exclusion criteria. T In some embodiments, as disclosed elsewhere herein, the same approach is used for donors, who have rigorously defined inclusion and exclusion criteria. Some embodiments, include one or more of the following steps: (1) patient screening/selection/baseline testing, (2) patient dosing with the mobilizing drug for 5 days consecutively, (3) collection of patient immune cells through leukapheresis, (4) transport to the cryoprocessing site, (5) processing, cryopreservation and short-term biobanking, (6) transport from the processing site to the biorepository, (7) long-term storage at the biorepository, (8) selection of a young donor for patient immune restoration through cell culture studies, (9) transport of the dose of cryopreserved patient and donor vials from the biorepository to the clinical site, (10) clinical manufacturing of the therapeutic at the clinical site, (11) patient treatment and (12) periodic patient testing for safety and efficacy. As an alternative, the process from step 1 through 7 could serve as an alternate business model as a bioinsurance company. In some embodiments, instead of using a donor, cell free processes (using exosomes, miRNA, or small molecules drugs as disclosed elsewhere herein) are used.

In some embodiments, the methods disclosed herein satisfy an aging population's desire for new therapies to extend their healthspan. In some embodiments, this is achieved through the immune system restoration therapy as disclosed herein. The immune system restoration therapy (or immune cell therapy) is an autologous treatment with no young factors infused, and there are no serious adverse events from this therapy detected in patients treated to date. In some embodiments, the immune system restoration therapy comprises, consists essentially of, or consists of 10⁵, 10⁶, 10⁷, 10⁸, 10⁹, or 10¹⁰ or about 10⁵, about 10⁶, about 10⁷, about 10⁸, about 10⁹, or about 10¹⁰ mobilized peripheral blood mononuclear cells after a 7-day transwell culture. In some embodiments, the immune system restoration therapy includes or comprises injection-grade saline (0.9% sodium chloride) and, optionally, 0.05% serum albumin. There have been several companies exploring young plasma infusions as an anti-aging therapy. This is not an autologous therapy and carries risks as well is unproven scientifically to be beneficial. Young plasma infusions are cell-free and contain water, dissolved proteins, glucose, clotting factors, hormones, exosomes, electrolytes, carbon dioxide, and oxygen. Potential side effects of young plasma infusions include transfusion-related acute lung injury, transfusion-associated circulatory overload, and allergic/anaphylactic reactions.

In some embodiments, testing occurs prior to treatment and then again at 1, 3, 6 9- and 12-months following treatment. In some embodiments, clinical manufacturing in the US and shipped the cell cultures to the clinical for final formulation. In some embodiments, this process banks the vials in the US and then transports the frozen vials to the clinic so that all clinical manufacturing is performed in a GMP clean room (e.g., at Okyanos). In some embodiments, the process uses a validated cold-chain logistics service (e.g., like Cryoport).

In some embodiments, the cold-chain shipping is used for transporting the freshly collected cells to a cryoprocessing site at 2-8 degrees C. (e.g., using a cyroshipper), and, in some embodiments, the cold-chain transport is used to transport the frozen vials of cells from the cryoprocessing site to the biorepository, and from the biorepository to the clinical site using dry liquid nitrogen shippers at −180 degrees C.

In some embodiments, as an alternative embodiment the process from part B, step 1 through 7 above, could serve as an alternate business model as a bioinsurance company. In some embodiments, vast amount of data to date that encompasses all steps above excluding steps 2 and 11. Of particular interest is the clinical data obtained from Quest for patient testing.

### Cyrogenic Blood Processing

In some embodiments, mobilized peripheral blood collected by leukapheresis yields a very high concentration of leukocytes, blood stem and progenitor cells. In some embodiments, more cells are collected then are needed for immune restoration therapy. In some embodiments, a clinical-grade method to cryogenically preserve the additional cells for long-term storage is used. In some embodiments, this method maintains cell viability and yield during the storage period (e.g., so that the thawed cell product is bioequivalent to the fresh product or of sufficient quality for use). In some embodiments, the method adds minimal processing to the freshly collected mobilized blood, so as to lower cell loss and preserve the heterogeneity of immune populations present in the matrix. In some embodiments, the method is free of any animal-derived components, thus making it xeno-free and clinical grade. In some embodiments, all reagents utilized in the proprietary cryogenic medium are also GMP grade. In some embodiments, a controlled rate freezing program is utilized. In some embodiments, the program is custom and carried out with a controlled rate freezer to freeze the cells at an average rate of −1 degrees C. per minute from a starting temperature of 2-8 degrees C. to −100 degrees C. prior to deposition into a liquid nitrogen dewar. In some embodiments, the current use of mobilized peripheral blood is primarily for cell transplantation studies clinically. In some embodiments, clinics often solely add cryoprotectant (DMSO) directly to the collection bag and then freeze the whole blood bag. When the bag is thawed for transfusion, the whole product including DMSO is infused into the patient. In some embodiments, the method disclosed herein uses a cryogenic medium partly containing DMSO as well as other factors to produce a highly viable cell product post thaw. In some embodiments, the cryogenic medium disclosed herein is not infused into the patient (advantageously). In some embodiments, the cryogenic medium may be supplemented with one or more of HSA and/or DMSO in amounts of equal to or less than about: 0.1%, 0.5%, 0.75%, 1.5%, 5%, 8%, 12.5%, 15%, 25%, 50%, or ranges spanning and/or including the aforementioned values (by wt % or % by volume).

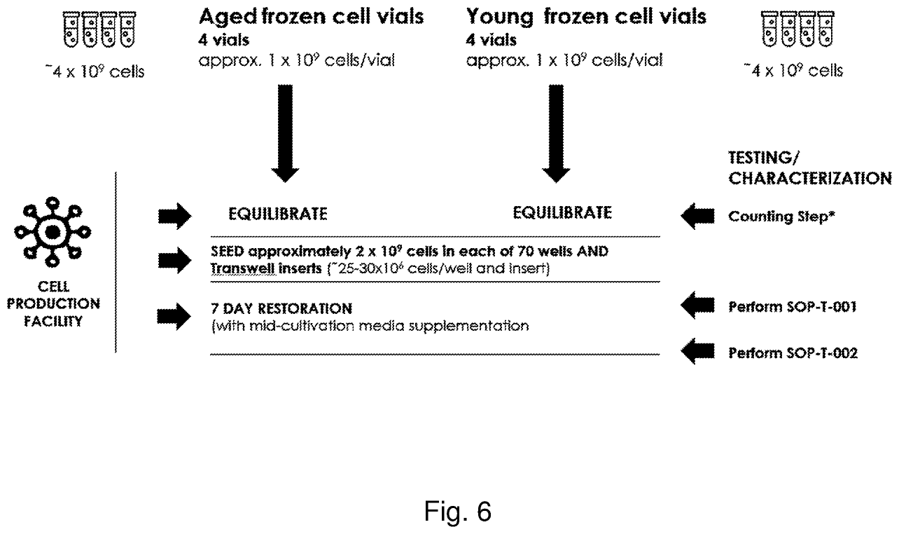

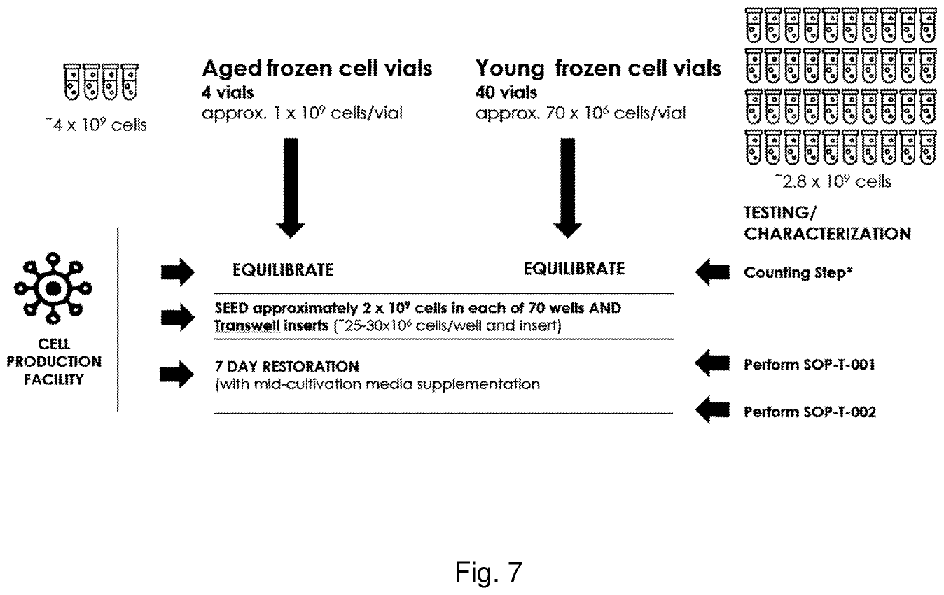

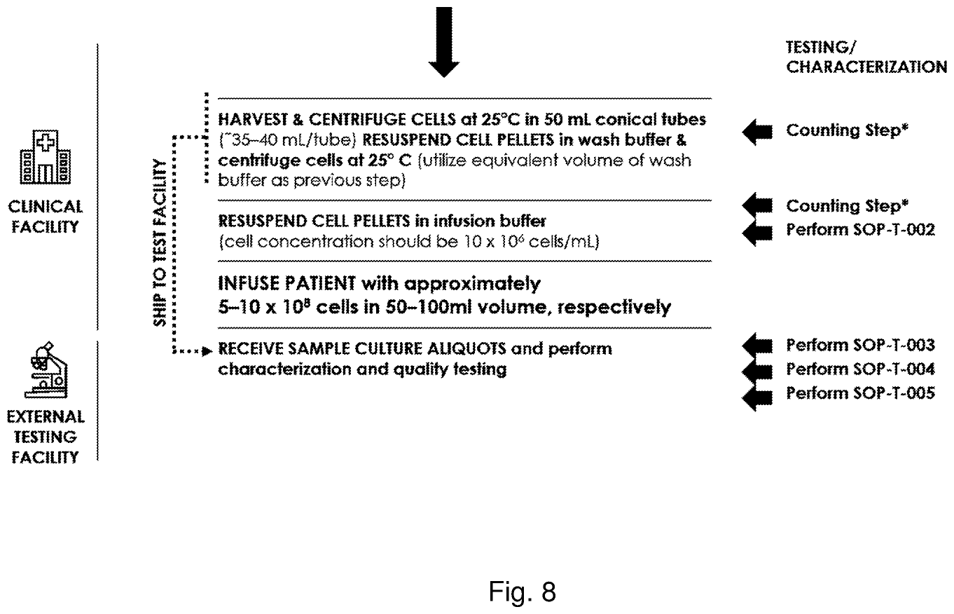

In some embodiments, the cryogenic method is a stepwise process starting with the initial product being a mobilized peripheral blood bag of approximately 400 cc volume from either a donor or a patient. In some embodiments, the final product is a number (70-80) of cryogenic vials of frozen cell product of approximately 5 cc each (e.g., as shown in FIGS. 6 and 7). In some embodiments, the steps in between encompass a volume reduction step, a wash step, a final resuspension in cryogenic medium step and a controlled rate freezing step. In some embodiments, described herein is a clinical-grade, custom cryogenic protocol to process, freeze and vial mobilized peripheral blood for long-term storage and/or autologous use therapeutically.

In some embodiments, advantages are the custom cyrogenic medium that we have formulated and the custom controlled rate freezing program. In some embodiments, the combination of these two yields a superior cell product for long-term storage. In some embodiments, when thawed, cell recovery, viability and potency are improved. In some embodiments, current standard protocol for clinical cryopreservation of mobilized blood for stem cell transplantations result in a thawed cell produced of considerably lesser viability. The most frequent use of mobilized peripheral blood clinically is for stem cell transplants in humans. The process described above cites that clinicians simply add cryoprotectant alone to the blood bag and freeze the product. When thawed at the patient's side the product is of lesser viability and contains the cryoprotectant, DMSO, infused into the patient intravenously because no processing steps are performed to alter the product volume or wash the cells to remove the cryoprotectant.

In some embodiments, the cryogenic medium is clinical-grade: all components are GMP certified and animal/xeno-free. There are other animal/xeno-free cGMP certified cryogenic media on the market, however, these have not been tailored to the processes disclosed herein. In some embodiments, the yield of cells from the unique cell collection type, which is mobilized peripheral blood, is improved. In some embodiments, the combination of the process/protocol with the cryogenic formulation and controlled-rate freezing program improves yield and quality of cells.

In some embodiments, the protocol includes a volume reduction and washing steps. In some embodiments, this process is performed using a controlled rate freezer. In some embodiments, a Forma Controlled Rate Freezer from Thermo Scientific is used. In some embodiments, the protocol can be adapted for optimal cryogenic processing and storage of other primary human cell and tissue types. In some embodiments, other relevant cell and tissue types include but are not limited to: umbilical cord blood mononuclear cells, umbilical cord mesenchymal stem cells, stromal vascular fraction isolated from adipose, adipose-derived stem cells, bone marrow aspirate, bone marrow mononuclear cells, bone marrow mesenchymal stem cells and endothelial progenitor cells. In some embodiments, the protocol can preserve recovery, viability and regenerative function post-thaw of each of these cell types. In some embodiments, modifications of the cryogenic medium, the volume depletion/wash steps or the controlled rate freezing program to can be made for each cell type. In some embodiments, data shows how the various iterations affected the post-thaw cell recovery (yield), viability and potency.

In some embodiments, the purpose of this method is to produce an efficient and expeditious protocol for cryogenic processing of G-CSF-mobilized patient Leukopaks that will preserve the cell product, while incorporating protocol modifications to enable compatibility with regulatory guidelines and medical application.

In some embodiments, materials used for the process include one or more of: 5 mL Cryogenic vials; Cryogenic gloves; Cryogenic storage boxes; Sterile 500 mL bottles; Chilled bead bucket; Sterilized scissors; 5 mL vial racks/holders; 50 mL conical tubes. In some embodiments, include one or more of: Human Serum Albumin (H.S.A.); Dimethyl Sulfoxide (DMSO); Normal Saline. In some embodiments, equipment includes one or more of: Control rate freezer; Label maker; Microcentrifuge; LN2 tank (Enough to run CRF).

In some embodiments, a cryogenic medium comprising HSA, DMSO, and saline is prepared. In some embodiments, place solution at 4° C. until ready to be used. In some embodiments, the temperature in the CRF chamber should be programmed to different temperature and cooling rates.

In some embodiments, freezing of TNCs from Mobilized Peripheral Blood is accomplished using one or more of the following steps. In some embodiments, make sure the centrifuge has been equilibrated to 4° C. before processing the Leukopaks. In some embodiments, label cryogenic vials with cell type, patient number, number of cells/mL, date and operator's initials. In ascending order, organize the cryogenic vial numbers into clean racks. In some embodiments, label cryogenic boxes with “rack number” location and “box number” (R#B#). In some embodiments, turn and leave on laminar flow hood, sterilize working surfaces with 70% ethanol and UV for a minimum of 10 minutes. In some embodiments, place labeled cryogenic boxes and labeled cryogenic vials under the laminar flow hood then turn on UV for a minimum of 20 minutes. In some embodiments, place the cryogenic vials in their designated boxes and place labeled cryogenic boxes (containing the labeled cryogenic vials) in the fridge for 15 minutes. In some embodiments, ethanol spray and wipe down the chilled bead bucket and place under the laminar flow hood. In some embodiments, take out the Leukopaks from the shipping container and spray with ethanol thoroughly, wipe down and place under the sterile laminar flow hood. In some embodiments, with sterile scissors, cut the top portion of the Leukopaks and transfer 40 mL (or equivalent to 1/10th of total Leukopak volume) of mobilized peripheral blood each into 10×50 mL ster-ile conical tubes. Spin down at 300 g, at 4° C., for 10 minutes to pellet. In some embodiments, remove 50% (approximately 20 mL) of supernatant from each of the 10×50 mL conical tubes until left with only 50% (approximately 20 mL) of the initial total volume of supernatant and cell pellets. In some embodiments, lightly tap and loosen the pellets for all 10×50 mL conical tubes, and then carefully resuspend and transfer the cell suspension into a single, sterile 500 mL bottle (total volume should be approximately 200 mL). In some embodiments, keep the cell suspension chilled by placing the 500 mL bottle in the bucket with the cold beads. In some embodiments, drop-wise add 200 mL (or a volume equal to the volume in the 500 mL bottle) of chilled cryogenic media into the cell suspension while gently shaking the bottle. This is a 1:1 dilution, with HSA and DMSO. In some embodiments, after gently mixing the cells with the cryogenic media, take a 1 mL aliquot for cell counting with Turk's solution and Trypan Blue exclusion. The cell count, total volume, cell concentration, and cell viability for the cell suspension is recorded.

Record cell number, volume, concentration and viability in table below.

# Cells:
Total Volume:
Cell Concentration:
Cell Viability:

In some embodiments, aliquot the cryogenic suspension into 5 mL aliquots within cryogenic vials. Place vials back into designated boxes and place at 4° C. for 15 minutes. Next, transfer to the controlled rate freezer and follow already programmed protocol. In some embodiments, transfer the boxes of cryogenic vials into the liquid phase of a liquid nitrogen dewar for long term cryopreservation.

### Additional Embodiments

In an embodiment, the subject is administered the compositions disclosed herein in a therapeutically effective amount sufficient for treating, preventing, and/or ameliorating one or more symptoms of a medical condition, disorder, disease, or dysfunction. Hereinafter, for simplicity, the unwanted condition which has been used interchangeably with the terms medical condition, disorder, disease, and dysfunction are collectively referred to as the “medical condition.” As used herein, amelioration of the symptoms of the medical condition by administration of a particular composition of the type disclosed herein refers to any lessening, whether lasting or transient, which can be attributed to or associated with administration of compositions of the type disclosed herein. As used herein, a “therapeutically effective amount” means a sufficient amount of the compositions disclosed herein to treat, prevent, and/or ameliorate one or more symptoms of the medical condition. It also may include a safe and tolerable amount of the compositions disclosed herein, as based on industry and/or regulatory standards. As will be understood by the ordinarily skilled artisan an amount that proves to be a “therapeutically effective amount” in a given instance, for a particular subject, may not be effective for 100% of subjects similarly treated for the medical condition under consideration, even though such dosage is deemed a “therapeutically effective amount” by ordinarily skilled practitioners. The therapeutically effective amount for a particular individual may vary depending on numerous factors such as the nature of the medical condition, severity of the medical condition, subject weight, subject age, and the general health of the subject. It is contemplated that the therapeutically effective amount may be optimized by one or more healthcare professionals in consideration of the particular factors affecting a subject.

One or more compositions disclosed herein may comprise cells and/or cellular material obtained from a human subject. Herein the term “cellular material” refers to materials derived from, secreted by, and otherwise currently or previously associated with a cell.

In some embodiments, the method for providing a target cell includes, a method of the present disclosure comprises (i) obtaining a donor cell sample and a receiver cell sample; (ii) utilizing one or more analytical techniques to characterize the donor cell sample and receiver cell sample; (iii) contacting one or more components of the donor cell sample with the receiver cell sample to generate a restored cell sample; (iv) utilizing one or more analytical techniques to characterize the restored cell sample; and (v) utilizing the restored cell sample for treatment of a subject.

In an alternative embodiment, a method of the present disclosure comprises (i) obtaining a donor cell sample and a receiver cell sample; (ii) contacting one or more components of the donor cell sample with the receiver cell sample to generate a restored cell sample; and (iii) utilizing the restored cell sample for treatment of a subject.

In yet another embodiment, a method of the present disclosure comprises one or more of obtaining a first cell sample from a first subject (e.g., a donor); obtaining a second cell sample from a second subject (e.g., a patient); culturing the first cell sample in the presence of at least a portion of a culture media of the second cell sample for a time period (e.g., ranging from about 24 hours to about 6 weeks) to produce a restoring composition. In some embodiments, the method includes contacting the restoring composition with the second cell sample for a period of time ranging from about 24 hours to about 6 weeks to produce a restored composition. In some embodiments, the second cell is introduced to the second subject. In some embodiments the first subject (e.g., the donor) is the second subject (e.g., the patient) at an earlier age. In some embodiments, the first subject has an age of less than or equal to 20, 25, 30, 35, 40 or ranges including and/or spanning the aforementioned values. In some embodiments, the second subject has an age of greater than or equal to 35, 40, 45, 50, 55, 60, 65, 70, 75, or ranges including and/or spanning the aforementioned values.

In still yet another aspect, a method of the present disclosure comprises (i) obtaining a first cell sample from a first receiver subject; (ii) obtaining a second cell sample from a second donor subject; (iii) culturing the first cell sample in the presence of at least a portion of a culture media of the second cell sample for a time period ranging from about 24 hours to about 6 weeks to produce a restored composition (v) obtaining a third cell sample from a third receiver subject and (vi) culturing the third cell sample in the presence of at least a portion of the culture media of the restored composition for a time period ranging from about 24 hours to about 6 weeks to produce a secondarily restored composition. In such aspects the first receiver subject and the third receiver subject are the sources of aged adult stem cells found in the first and third cell samples, respectively. Further in such aspects, the second donor subject who provides the second cell sample is characterized as chronologically younger than the first or third receiver subjects. In some embodiments, the secondarily restored composition may be utilized in a like manner to produce a tertiary restored composition and so forth.

In an embodiment, the donor cell sample is provided by a donor subject while the receiver cell sample is provided by a receiver subject. In some embodiments, the donor subject and receiver subject are the same. Alternatively, the donor subject and receiver subject are different. In an embodiment, the donor subject is chosen such that the difference in the age of the donor subject, designated x, and the age of the receiver subject, designated y, is greater than about 5 years, alternatively, greater than about 10 years, alternatively greater than about 15 years, alternatively greater than about 20 years, alternatively greater than about 25 years, or alternatively greater than about 30 years where y is greater than x. In an embodiment, the donor subject is chosen such that the difference in the age of the donor subject, x, and the age of the receiver subject, y, is from about 5 years to about 75 years, alternatively from about 10 years to about 60 years, alternatively from about 15 years to about 50 years, alternatively from about 20 years to about 40 years, or alternatively from about 20 years to about 30 years where y is greater than x.

In some embodiments, the difference in chronological age between the donor subject and receiver subject is equal to or greater than about 16 years, alternatively from about 16 years to about 80 years, alternatively from about 16 years to about 50 years, or alternatively from about 16 years to about 30 years and x is greater than y. In yet another embodiment, the difference in chronological age between the donor subject and the receiver subject is less than about 365 days.

In an embodiment, the donor subject and receiver subject are related by consanguinity. Alternatively, the donor subject and receiver subject are not related. In an embodiment, the receiver subject has a medical condition that is absent from or undiagnosed in the donor subject. In either of the above disclosed embodiments, the donor subject and the receiver subject are adults, i.e., have reached sexual maturity. Alternatively, in either of the above disclosed embodiments, the donor subject has reached sexual maturity. Alternatively, in either of the above disclosed embodiments, the receiver subject has reached sexual maturity.

In an embodiment, the receiver subject is identified as having one or more risk factors associated with the development of a medical condition. In yet another embodiment, the receiver subject has not been diagnosed with a medical condition and/or has not been identified as having one or more risk factors associated with the development of a medical condition. It is contemplated that the methodologies disclosed herein may be employed in the treatment of subjects having a medical condition for which additional therapies have been previously or are currently being employed. It is further contemplated that in an embodiment, a receiver subject has undergone or is currently undergoing one or more therapies for medical conditions not associated with the medical condition for which the subject will be treated using the compositions and methodologies disclosed herein. In an embodiment, the receiver subject has one or more age-related medical conditions.

In an embodiment, the donor cell sample, receiver cell sample, or both are obtained from a subject(s) who has undergone a Stage B preparation. In some embodiments, the donor cell sample, receiver cell sample, or both are obtained from a subject(s) who has undergone a Stage A preparation and a Stage B preparation.

In an embodiment, the donor cell sample, the receiver cell sample, or both are obtained from a subject that has undergone a Stage A preparation. Herein, a Stage A preparation of a subject comprises the utilization of methods and/or compositions to improve the subject's general health prior to obtaining a composition (i.e., donor cell sample or receiver cell sample) from the subject.

A nonlimiting example of a methodology to improve the subject's general health includes the administration of one or more metabolic mediators to the subject. Herein, metabolic mediator refers to a substance which, when present in insufficient amounts in the subject, is detrimental to the physiological and/or psychological state of the subject or whose presence positively impacts the physiological and/or psychological state of the subject. The subject may be administered a plurality of metabolic mediators prior to obtaining one or more compositions of the type disclosed herein from the subject.

In an embodiment, the metabolic mediator comprises a nutraceutical. Herein, a nutraceutical refers to a material that may be derived from a natural source and that provides health benefits. A nonlimiting example of a nutraceutical suitable for use in the Stage A preparation of a subject is commercially available as EVERYCELL®, HEALTHYCELL, or HEALTHYCELL PLUS from Cell Health Institute. Additional compositions suitable for use metabolic mediators in the present disclosure are described in U.S. Pat. No. 8,747,918 entitled “Dietary Supplement System for Multifunctional Anti-Aging Management and Method of Use” which is incorporated by reference herein in its entirety.

Another example of a methodology suitable for use in Stage A preparation of a subject comprises the administration of one or more pulsed electromagnetic fields (PEMF) to at least a portion of the subject's body prior to and/or concurrent with, obtaining a sample of the type disclosed herein. PEMF may be used to enhance the homing, engraftment, and/or differentiation of the adult stem cells.

Stage A preparation of a subject may be carried out for some period of time prior to, and/or concurrent with obtaining a cell sample of the type disclosed herein from the subject. For example, Stage A preparation of a subject may comprise administration of a nutraceutical to the subject at a particular dosage (e.g., 500 mg, twice daily) for a period of time greater than about 48 hours prior to obtaining a cell sample of the type disclosed herein from the subject. Alternatively, the nutraceutical is administered for a time period of from about 48 hours to about 1 year prior to obtaining a cell sample of the type disclosed herein from the subject, alternatively from about 1 week to about 9 months, or alternatively from about 1 month to about 6 months. In some embodiments, the subject may be administered or may self-administer the nutraceutical for any period of time prior to, concurrent with, or subsequent to the procurement of a cell sample.

In an embodiment, the donor cell sample, the receiver cell sample, or both are obtained from a subject that has undergone a Stage B preparation. In an embodiment, during a Stage B preparation, the subject (donor and/or receiver) undergoes at least one process for mobilizing the subject's stem cells. Herein “stem cells” are given their usual meaning which generally refers to cells which are not terminally differentiated and are therefore able to produce cells of other types. Stem cells are typically divided into three types, including totipotent, pluripotent, and multipotent. “Totipotent stem cells” can grow and differentiate into any cell in the body, and thus can grow into an entire organism. These cells are not capable of self-renewal. In mammals, the zygote and early embryonic cells are totipotent. “Pluripotent stem cells” are true stem cells, with the potential to make any differentiated cell in the body, but cannot contribute to making the extraembryonic membranes (which are derived from the trophoblast). “Multipotent stem cells” are clonal cells that self-renew, as well as differentiate, to regenerate adult tissues. “Multipotent stem cells” are also referred to as “unipotent” and can only become particular types of cells, such as blood cells or bone cells.

In an embodiment, the donor and receiver cell samples comprise adult stem cells and/or adult stem cell material which refer to stem cells or stem cell material that are not embryonic in origin nor derived from embryos or fetal tissue. In an alternative embodiment, the donor cell sample comprises adult stem cells and/or adult stem cell material which refer to stem cells or stem cell material that are not embryonic in origin or derived from embryos or fetal tissue. In an embodiment, the donor and receiver cell samples comprise stem cells and/or stem cell material that are embryonic in origin and/or derived from embryos or fetal tissue. In an alternative embodiment, the donor cell sample comprises stem cells and/or stem cell material that are embryonic in origin and/or derived from embryos or fetal tissue.

In an embodiment, Stage B preparation comprises administering to a subject an effective amount of a mobilizer. An effective amount of a mobilizer may be determined by the ordinarily skilled artisan consistent with best medical practices and taking into account a variety of factors including, for example and without limitation, the subject's general health and body mass.

As known to one of ordinary skill in the art, stem cells have been identified in various organs and tissues, including brain, bone marrow, peripheral blood, blood vessels, skeletal muscle, skin, teeth, heart, gut, liver, ovarian epithelium, and testis. It is contemplated that utilization of Stage B preparation of a subject would be carried out when obtaining stem cells using bone marrow as the source. It is within the scope of this disclosure to conduct various embodiments of the present methods using cell samples comprising stem cells obtained from any of the tissues known to be a source of stem cells. In such embodiments, Stage B preparation of the subject may not be carried out.

In an embodiment, a donor subject, a receiver subject, or both undergo Stage A preparation. In an embodiment, a donor subject, a receiver subject, or both undergo Stage B preparation. In an embodiment, a donor subject, a receiver subject, or both do not undergo Stage A preparation. In an embodiment, a donor subject, a receiver subject, or both do not undergo Stage B preparation. In an embodiment, a donor subject and a receiver subject, or both undergo Stage A and Stage B preparation.

Subsequent to administration of the mobilizer, and after a suitable time period has elapsed; a cell sample (e.g., donor cell sample or receiver cell sample) may be harvested from a subject. The time period between administration of the mobilizer to the subject and harvesting of the cell sample may be varied to meet one or more user and/or process goals. In an embodiment, the time period between administration of the mobilizer and harvesting of the cell sample may range from about 24 hours to about 10 days, alternatively from about 48 hours to about 7 days, or alternatively from about 3 days to about 5 days.

In an embodiment, the cell sample is harvested from a subject using any suitable methodology, for example, using an extracorporeal therapy such as apheresis. Apheresis is a method used to collect only a specific part of the subject's blood. It works on the basis of centrifugation or rapid spinning of the blood. A pathway is established for the subject's blood and allows for connection to the apheresis device. The instrument uses small pumps to move blood and fluids through the system. One pump draws blood out of one arm or side of the catheter and directs it to the centrifuge where the blood is separated into red cell, white cell, and plasma layers. A portion of the white cell layer, which includes stem cells, and a small amount of plasma and red cells are diverted to a collection bag. The rest of the blood is returned to the subject in the other arm or the second side of the catheter. In such an embodiment, the cell sample is harvested using intravenous needles located in a vein in each arm of a subject. Blood may be removed from a first vein, passed through an extracorporeal circuit that separates out the cell sample of interest and the remaining material may be returned to a second vein.

In an embodiment, the donor cell sample and/or receiver cell sample are harvested from the bone marrow directly. For example, the cell sample may be harvested from the iliac crest of a subject. In such embodiments, bone marrow aspiration to obtain the cell sample may involve a healthcare provider locating the posterior iliac crest of the subject subsequent to carrying out standard precautions such as skin sterilization and the administration of a local anesthetic. A suitable needle with the stylet in place may be slowly advanced through the skin and subcutaneous tissue pointing towards the anterior superior iliac spine. Upon reaching the posterior iliac crest, the area may be penetrated by the needle until an adequate depth is reached. Once the needle is in place, the stylet may be removed, a syringe attached, and the aspiration performed.

In an embodiment, a plurality of stem cell collections (e.g., bone marrow aspirations) is carried out in order to obtain some user and/or process desired number of cells in the cell sample. For example, the number of cells collected may range from 1×10⁶-1.0×10⁹ cells/kg of the subject weight, alternatively from about 2×10⁶-1.0×10⁸ cells/kg of the subject weight, or alternatively from about 5×10⁶-1.0×10⁸ cells/kg of the subject weight. Cell samples harvested as disclosed herein may be utilized without further processing in the methodologies disclosed herein. Alternatively, cell samples harvested as disclosed herein may be further processed using any methodology compatible with the compositions and methodologies disclosed herein. Alternatively, cell samples harvested as disclosed herein may be stored for some time period before being utilized in the methodologies and therapies disclosed herein. Storage of the cell samples may involve, for example, cryogenic preservation of the cell sample in a biocompatible solution to stabilize the sample for the duration of storage. “Biocompatible solution” refers to solutions in which the cell sample (e.g., donor and/or receiver) are suspended for use in the cellular restoration methodologies disclosed herein or for any other subsequent uses. Such biocompatible solutions may include saline and may further comprise other ingredients such as preservatives, antimicrobials, and the like.

In an embodiment, cell samples harvested as disclosed herein are stored for greater than about 24 hours prior to being utilized in the methodologies disclosed herein. Alternatively, the cell samples harvested as disclosed herein are stored for a period of time ranging from about 1 hour to about 20 years prior to being utilized in the methodologies disclosed herein. Alternatively, storage of a cell sample harvested as disclosed herein may be for a time period ranging from about 10 days to about 15 years, alternatively from about 30 days to about 10 years, or alternatively from about 30 days to about 5 years.

As will be understood by the ordinarily skilled artisan, the donor cell sample and/or receiver cell sample, as harvested, comprise a heterogeneous cell population. An aspect of the methodologies disclosed herein comprises identifying and quantifying the types and amounts of cells present in the donor cell sample and/or receiver cell sample. Any methodology suitable for characterizing the number and types of cells present in the donor cell sample and/or receiver sample may be employed. In an embodiment, the donor cell sample and/or receiver cell sample are characterized by immunophenotyping. Herein, immunophenotyping refers to the analysis of heterogeneous populations of cells for the purpose of identifying the presence and proportions of the various populations in the sample. Antibodies are used to identify cells by detecting specific antigens (termed markers) expressed by these cells. In an embodiment, the donor cell sample and/or receiver sample are characterized by immunophenotyping using techniques such as flow cytometry. In alternative embodiments, characterizations of the various cell types present in a donor cell sample and/or receiver cell sample may be carried out using any suitable methodology such as reverse transcriptase polymerase chain reaction (RT-PCR) or immunocytochemistry.

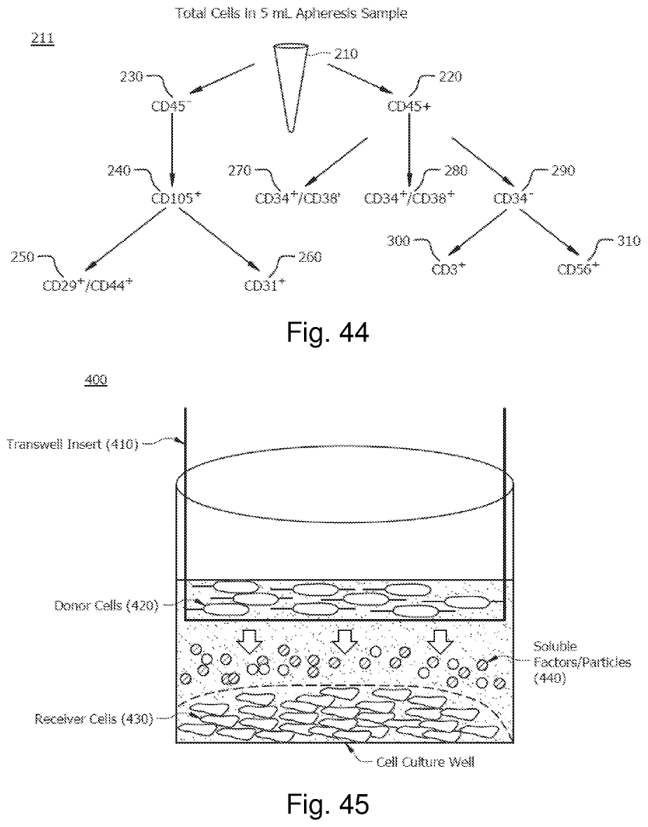

In an embodiment, the populations of cells or cell types present in the donor cell sample and/or receiver cell sample are identified based on the presence or absence or one or more cell surface markers. An embodiment of a flow cytometry protocol for the identification of the different populations of cells (e.g., cell types) in a donor cell sample and/or receiver cell sample, 211, is presented in FIG. 44. Referring to FIG. 44, a cell sample (donor and/or receiver sample) 210 is subjected to flow cytometry. In an embodiment, the donor cell sample and/or receiver cell sample 210 may be, at a first stage, sorted into hematopoietic cells 220 and non-hematopoietic cells 230 based on the presence or absence of CD45. CD45, also known as leukocyte common antigen (LCA), T200, B220, Ly5, and protein tyrosine phosphatase receptor type C (PTPRC) is a transmembrane glycoprotein of the leukocyte-specific-receptor-like protein tyrosine phosphatase family. It is expressed on all nucleated hematopoietic cells and can cover up to 10% of the cell surface area. CD45 functions as a regulator of T-cell and B-cell antigen receptor signaling and is a regulator of cell growth and cell differentiation.

In an embodiment, CD45− cells, identified as non-hematopoietic stem cells 230, may be further characterized on the basis of the presence or absence of CD105. CD105, also known as endoglin, HHT1, ORW, and SH-1 is a type I membrane glycoprotein located on cell surfaces and is a component of the TGFβ receptor complex. CD105 may play a role in hematopoiesis and angiogenesis. In an embodiment, a cell population that is both CD45− and CD105+, 240, is characterized as having both mesenchymal stem cells and endothelial progenitor cells.

In an embodiment, a cell population that is identified to be both CD45− and CD105+, 240, may be further sorted into mesenchymal stem cells and endothelial progenitor cells. In an embodiment, the mesenchymal stem cells are identified as being CD45−, CD105+, CD29+ and CD44+, 250. CD29, also known as platelet GPIIa, integrin β1, and GP is an integrin unit associated with very late antigen receptors and functions in cell adhesion. CD44, also known as ECMRII, H-CAM, Pgp-1, HUTCH-1, Hermes antigen, phagocytic glycoprotein I, extracellular matrix receptor III, GP90 lymphocyte homing/adhesion receptor, and hyaluronate receptor functions in cell adhesion and migration. In an embodiment, endothelial progenitor cells are identified as being CD45-, CD105+, and CD31+, 260. CD31, also known as PECAM-1, endoCAM, platelet endothelial cell adhesion molecule, and PECA-1 is a protein that in humans is encoded by the PECAM1 gene found on chromosome 17. CD31 is thought to function in cell adhesion, activation, and migration.

The method of the present disclosure may further comprise identifying the differing hematopoietic cell types present in the CD45+ cells, 220. In an embodiment, a population of the cells is identified as being primitive hematopoietic stem cells, 270, on the basis of being CD45+, CD34+ and CD38-. In an embodiment, a population of the cells is identified as being hematopoietic progenitor cells on the basis of being CD45+, CD34+ and CD38+, 280. CD34 also known as gp105-120 and hematopoietic progenitor cell antigen (HPCA-1) is a member of the family of single-pass transmembrane sialomucin proteins that are expressed on early hematopoietic and vascular tissues. CD34 is thought to function in cell adhesion. CD38, also known as ADP-ribosyl cyclase, T10, and cyclic ADP-ribose hydrolase 1 is a multifunctional ectonucleotidase encoded by the CD38 gene which is located on chromosome 4. In an embodiment, at least a portion of the cell population are CD45+ and CD34-, 290, and are identified as differentiated hematopoietic cells. In such an embodiment, the differentiated hematopoietic cells, 290, may be further defined as being T-lymphocytes, 300, or Natural Killer cells, 310. T-lymphocytes can be characterized as being CD45+, CD34-, and CD3+. CD3, also known as T3, is a protein complex and plays a role in cell adhesion between T-cells and other cell types. Natural Killer cells can be characterized as being CD45+, CD34-, and CD56+. CD56 also known as Leu-19, NKH-1, and neural cell adhesion molecule (NCAM) is a hemophilic binding glycoprotein that may function in cell-cell adhesion, neurite outgrowth, synaptic plasticity, and learning and memory.

In an embodiment, the donor cell sample and/or receiver sample may be characterized using the methodologies disclosed herein. Such characterizations may result in the identification of cell populations in the donor cell sample and/or receiver cell sample that include without limitation, non-hematopoietic cells, mesenchymal stem cells, endothelial progenitor cells, hematopoietic cells, primitive hematopoietic stem cells, hematopoietic progenitor cells, differentiated hematopoietic cells, T-lymphocytes, natural killer cells, or combinations thereof. It is contemplated that the surface markers described herein represent one methodology for the identification of cell populations present within the donor cell sample and/or receiver cell sample. As will be understood by the ordinarily skilled artisan, numerous markers and combination of markers other than those disclosed herein may be utilized to identify and characterize the cell populations present within the donor cell sample and/or receiver cell sample. Further, the identification of the various cell populations present in the donor cell sample and/or receiver cell sample may be carried out to the extent described herein, may include determination of the presence or absence of additional surface markers, may utilize fewer markers than disclosed herein, or may be carried out to a lesser extent such that fewer populations of cells within the donor cell sample and/or receiver cell sample are identified. In an embodiment, a method comprises excluding the identification of the different populations of cells present in a donor cell sample and/or receiver cell sample.

In an embodiment, a donor cell sample and/or receiver cell sample is obtained from a subject having undergone a Stage B preparation. In such embodiments, the donor cell sample and/or receiver cell sample may be further characterized based on the number of senescent cells and non-senescent cells present in the cell sample. Herein, non-senescent cells refer to the cells that retain the ability to divide many times over without showing replicative senescence. Herein senescent cells refer to cells having a long-term loss of proliferative capacity despite continued viability and metabolic activity.

Senescent cells may be identified using a variety of metrics that include for example loss of proliferation, morphological changes, decreased telomere lengths, increased S-β-GAL activity, the production of senescence-associated heterochromatic foci (SAHF), increased production of senescence-associated secretory factors (SASF), increased production of reactive oxygen species (ROS), increased DNA damage, decreased chaperone-mediated autophagy, or combinations thereof. It is contemplated that changes in the various metrics described are assessed relative to comparable cell types established to be non-senescent cells. Alternatively, the characteristics of the cell sample may be compared to literature values established for the analyzed metric in a corresponding non-senescent cell.

Non-senescent cells may characterized by the length of their telomeres and of the level of telomerase activity present in the cell. By way of a non-limiting example, non-senescent cells present in the donor cell sample may be characterized by telomere lengths greater than or equal to about 4 kilobases, alternatively 4.5 kilobases, or alternatively 5 kilobases. It will be understood by the ordinarily skilled artisan that teleomere lengths indicative of non-senescent cells may vary depending on the cell type. Consequently, for a particular cell type, the telomere length characteristic of a non-senescent cell may be determined by routine experimentation.

In an embodiment, Stage B preparation of the subject from which the donor cell sample and/or receiver cell sample is harvested results in the preferential mobilization of non-senescent cells. The result of the preferential mobilization of non-senescent cells may be a donor cell sample and/or receiver cell sample comprising greater than 90% non-senescent cells, alternatively greater than 91% non-senescent cells, alternatively greater than 92% non-senescent cells, alternatively greater than 93% non-senescent cells, alternatively greater than 94% non-senescent cells, alternatively greater than 95% non-senescent cells, alternatively greater than 96% non-senescent cells, alternatively greater than 97% non-senescent cells, alternatively greater than 98% non-senescent cells, or alternatively greater than 99% non-senescent cells. The percentage of non-senescent cells is based on the total number of cells present in the sample. In an embodiment, the donor cell sample and/or receiver cell sample comprise from about 90% non-senescent cells to about 99% non-senescent cells based on the total number of cells present in the sample.

In some embodiments, the non-senescent cells present in the donor cell sample and/or receiver cell sample may be identified using any suitable methodology. In such embodiments, the non-senescent cells may be separated from the senescent cells using any suitable process compatible with the present disclosure to result in a donor cell sample and/or receiver cell sample that comprises, consists essentially of, or consists of non-senescent cells. It is contemplated that such methodologies may be extended to further define a population of non-senescent cells having the presence or absence of particular cell surface markers and result in a donor cell sample and/or receiver cell sample comprising, consisting essentially of, or consisting of non-senescent cells of a particular type (e.g., non-senescent mesenchymal stem cells, non-senescent natural killer cells).

In an embodiment, the donor cell sample and/or receiver cell sample may be analyzed for the extent of expression of one or more genes and/or proteins associated with cellular senescence. Such analyses may be carried out using a restoration biomarker protein panel (RBPP) and/or restoration biomarker gene expression panel (RBGEP) of the types disclosed herein.

In an embodiment, the RBPP comprises a plurality of antibody probes for factors linked to cellular aging and senescence. For example, the RBPP may comprise greater than 5 antibody probes, alternatively greater than 10 antibody probes, or alternatively greater than 20 antibody probes. In an embodiment, the RBPP comprises from 10 to 15 antibody probes. An example of a RBPP suitable for use in this disclosure is a protein array panel designated RBPP-X1 comprising antibody probes to the proteins listed in Table 1:

TABLE 1

| Name | Also Known As | Designated |
|---|---|---|
| granulocyte-colony | colony-stimulating | G-CSF |
| stimulating factor | factor 3 | |
| chemokine ligand 26 | eotaxin-3, macrophage | CCL26 |
| | inflammatory protein | |
| | 4-alpha, thymic stroma | |
| | chemokine, and IMAC | |
| hepatocyte growth factor | hepatocyte scatter | HGF |
| | factor (HSF), | |
| insulin-like growth factor | placental protein 12 | IGFBP-1 |
| binding protein 1 | (PP12) | |
| insulin-like growth factor | | IGFBP-4 |
| binding protein 4 | | |
| insulin-like growth factor | | IGFBP-6 |
| binding protein 6 | | |
| insulin-like growth factor beta | catabolin | IL-β |
| macrophage inflammatory protein 3 | chemokine ligand 20, | MIP-3α |
| (MIP3A) | liver activation regulated | |
| | chemokine (LARC) | |
| stem cell factor | KIT-ligand, KL, | SCF |
| | steel factor | |
| thymus and activation | chemokine ligand 17 | TARC |
| regulated chemokine | (CCL17), | |
| transforming growth factor beta 1 | | TGF-β1 |
| tumor necrosis factor receptor | | sTNFR1 |
| superfamily member 1A | | |
| vascular endothelial growth factor | | VEGF |

In some embodiments, the RBGEP may comprise greater than 5 gene probes, alternatively greater than 10 gene probes, or alternatively, greater than 20 gene probes. In some embodiments, the RBGEP comprises from 10 to 15 gene probes. In some aspects, the RBGEP comprises gene probes for factors linked to the regulation of cell cycle or the p53 pathway such as IFBP3, CSC25C, ABL1, CDKN2B, ALDH1A3, SIRT1, ING1, CITED2, CDKN1C, or a combination thereof. The RBGEP may further comprise gene probes for factors associated with regulation of inflammatory processes such as CDKN1A, IRF3, EGR1, IFNG, CDKN1B, NFKB1, SERPING2, IGFBP7, IRF7, or a combination thereof. The RBGEP may further comprise gene probes for factors associated with regulation of DNA damage related-processes such as PCNA, TERT, TP53BP1, or a combination thereof. The RBGEP may further comprise gene probes for factors associated with oxidative stress such as PRKCD, SOD1, NOX4, or a combination thereof. The RBGEP may further comprise gene probes for factors associated with cellular senescence such as CDKN2A, CDK6, TWIST, ATM, CCND1, ETS2, RBL2, BMI1, ETS1, or a combination thereof. The RBGEP may further comprise gene probes for factors associated with the MAPK pathway such as HRAS, MAP2K3, or both. The RBGEP may further comprise gene probes for factors associated with cytoskeletal function such as VIM, PIK3CA, THBS1, or a combination thereof. The RBGEP may further comprise gene probes for factors associated with the p16 effector pathway such as TBX3, TBX2 or a combination thereof. The RBGEP may further comprise gene probes for factors associated with insulin signaling such as IGFBP5. The RBGEP may further comprise gene probes for factors associated with cell adhesion such as CDL3A1, CD44, TGFB1A, CDL1A1, TGFB1 or a combination thereof. The RBGEP may further comprise gene probes for factors associated with the p53 effector pathway such as E2F1, MYC or both. An example of a RBGEP suitable for use in this disclosure, designated RBGEP-X1, is a gene panel comprising cDNA to the proteins listed in Table 2:

TABLE 2
RBGEP-X1 gene panel

| Gene | Protein Encoded |
|---|---|
| IGFBP3 | insulin-like growth factor binding protein 3 |
| HRAS | Transforming protein p21 |
| PRKCD | protein kinase C delta |
| AKT1 | alpha serine/threonine protein kinase |
| CHEK2 | checkpoint kinase 2 |
| MAPK14 | mitogen-activated protein kinase 14 |
| IGF1 | insulin-like growth factor |
| TWIST1 | Twist-related protein 1 |
| CDC25C | M-phase inducer phosphatase 3 |
| CCNA2 | cyclin-A2 |
| CDK5 | cell-division protein kinase 6 |
| CCNE1 | G1/S-specific cyclin E1 |
| CHEK1 | checkpoint kinase 1 |

In an embodiment, at least a portion of the donor cell sample and/or receiver cell sample are subjected to protein array analyses utilizing the RBPP-X1 array, gene expression analysis using the RBGEP-X1 array, or both. In alternative embodiments, at least a portion of the donor cell sample and/or receiver cell sample are subjected to protein array analyses, gene expression analyses or both utilizing any suitable protein and/or gene array.

In an embodiment, the donor cell sample, receiver cell sample, or both are subjected to at least one analytical technique to characterize the quality of the cell sample. Herein, the “quality” of the cell sample refers to factors used to characterize the cellular health of the sample and includes parameters such as the number and types of cells present in the sample; the ratio of senescent to non-senescent cells in the sample; the extent of expression of a group of genetic and/or protein biomarkers; the average telomere length of the cells in the sample; and the status of the innate immune function of the cells in the sample. Telomere length may be determined using any suitable methodology, for example, terminal restriction fragment (TRF) analysis. Innate immune function may be evaluated using any suitable methodology such as the ⁵¹Cr cytotoxicity release natural killer cell assay. The donor cell sample quality may be an assessment of the ability of the cells in the sample to improve and/or restore one or more cellular functions of the cells in the receiver cell sample. The receiver cell sample quality may be an assessment of the ability of the cells in the sample to exhibit improvement and/or the restoration of one or more cellular functions when subjected to the compositions and methodologies disclosed herein.

The donor cell sample quality may be assigned a numerical value that ranges from 1 to 10 wherein a sample displaying positive characteristics for use in the improvement and/or restoration of cellular function of a receiver cell sample has a value of 10, and a sample exhibiting the fewest characteristics associated with the ability to improve/restore cellular function of a receiver cell sample has a value of 1. For example, each of the following factors may weigh positively in characterization of the quality of a donor cell sample; relatively long telomere length; high level of expression of cell viability-promoting genes and/or proteins; the presence of greater than about 90% non-senescent cells; and high levels of innate immune function. Donor cell samples displaying these characteristics may be given a sample quality value of 10.

The receiver cell sample quality may be assigned a numerical value that ranges from 1 to 10 wherein a sample having restorable or improvable cellular function has a value of 10, and a sample whose cellular function cannot be significantly improved and/or restored has a value of 1. For example, each of the following factors may weigh positively in characterization of the quality of a receiver cell sample; relatively long telomere length; moderate level of expression of senescence-promoting genes and/or proteins; and the presence of greater than about 90% non-senescent cells. Receiver cell samples displaying these characteristics may be given a sample quality value of 10.

Utilizing the quality metrics disclosed herein (e.g., telomere length, percentage of non-senescent cells), an aspect of the present disclosure comprises evaluating the quality of the donor cell sample and receiver cell sample and identifying samples suitable for use in the disclosed methodologies. For example, a receiver cell sample having a quality value of less than 3 may be deemed unsuitable for use in the presently disclosed methodologies. Similarly, a donor cell sample having a quality value of less than 3 may be deemed unsuitable for use in the present methodologies. In some embodiments, the a donor cell sample having a quality value of equal to or greater than 7 may be used in the methodologies disclosed herein with a receiver cell sample having a quality value of equal to or greater than 7. It is to be understood that the quality values may be assigned based on any number of metrics used to assess the quality of a donor cell sample and/or receiver cell sample. Consequently, based on the parameters used to make the assignment of a quality value, the characteristics associated with a particular quality value may differ.

In some embodiments, the donor cell sample and/or receiver cell sample having been subjected to one or more of the qualitative and quantitative characterizations described herein are further processed to provide some user and/or process desired sample containing a predetermined type and number of cells. The present disclosure contemplates the utilization of such characterized samples. For example, the characterized samples may be a component of a pharmaceutical formulation that is administered to a subject to ameliorate one or more medical conditions.

Alternatively, the donor cell sample and receiver cell sample may be utilized in the restoration methodologies disclosed herein.

In an embodiment, a method of cellular restoration comprises contacting the soluble factors and/or particles present in the media of a cultured donor cell sample with the receiver cell sample. For example, the donor cell sample may be cultured in appropriate media for a time period ranging from about 24 hours to about 6 weeks, alternatively, from about 1 week to about 5 weeks or alternatively, from about 2 weeks to about 4 weeks. Herein, the culture media, also known as the growth media, refers to a liquid or gel containing the appropriate nutrients to support the growth of cells. Suitable culture media may be chosen by the ordinarily skilled artisan with the benefits of the present disclosure. The culture media may then be removed from the donor cell sample using any suitable methodology (e.g., filtration, centrifugation) and the cell-free media then contacted with the receiver cell sample.

In an alternative embodiment, the method of cellular restoration comprises establishing a transwell culture of both the donor cell sample and receiver cell sample. Referring to FIG. 45, the transwell culture 400 may comprise an insert 410 having at least one permeable surface that allows the donor cells to uptake and secrete molecules on the basal and/or apical surfaces of the transwell. The transwell insert 410 may be comprised of any material compatible with the compositions and methodologies disclosed herein such as, for example, polyethylene terephthalate or polycarbonate. In an embodiment, the transwell insert 410 comprises a permeable membrane with a pore size ranging from 0.4 μm to 3.0 μm, alternatively from 0.4 μm to 2.0 μm, or alternatively from 0.4 μm to 1.0 μm. The transwell insert may have a pore size that allows for the passage of soluble factors and/or particles secreted or released from the donor cell sample to the lower compartment of the transwell where these materials contact the receiver cell sample. At least a portion of the donor cell sample 420 may be applied to the transwell insert 410 while the receiver cell sample 430 is positioned within the lower compartment of the transwell culture with an appropriate amount of culture media. The donor cell sample and receiver cell sample may be cultured in the transwell for a time period of from 24 hours to 6 weeks, alternatively, from 1 week to 5 weeks, or alternatively, from 2 weeks to 4 weeks. Soluble factors and/or particles of the appropriate size 440 are allowed to pass through the permeable membrane and contact the receiver cell sample 430 in the lower chamber of the transwell.

In an embodiment, the donor cell sample and receiver cell sample are recovered separately from the transwell culture. The donor cell sample, as recovered from the transwell culture, is hereinafter termed the altered donor cell sample (ADCS). The receiver cell sample as recovered from the transwell culture is termed the restored composition (RC). In an embodiment, the ADCS and/or RC may be further characterized using any of the methodologies disclosed herein. In some embodiments, at least a portion of the ADCS and/or RC are further processed, for example, the samples may be prepared for cryopreservation. In yet another embodiment, at least a portion of the RC is utilized to treat a subject.

Herein the RC refers to the cellular material subsequent to culturing with the soluble factors of the donor cell sample for the time periods disclosed herein. The RC is characterized by improvement in one or more of the following metrics when compared to the receiver cell sample; innate immune function, morphology, colony-forming ability, reduced expression of senescence-promoting factors; increased expression of cell-viability promoting factors, and the like. In an embodiment, the RC is characterized by the presence of cells having gene expression and protein expression patterns for cellular senescence associated agents (e.g., CDKN2A, CDK6, TWIST, ATM, CCND1, ETS2, RBL2, BMI1, and ETS1) that are quantitatively more similar to those of the donor cell sample than the receiver cell sample.

In one embodiment, a methodology disclosed herein comprises the preparation of a RC. The RC is derived from the receiver cell sample that is subjected to the methodologies disclosed herein, specifically by the restoration of at least a portion of the receiver cell sample. Herein, “restoration” refers to modification of a cell (e.g., stem cell) such that expression of one or more senescence-promoting agents is reduced and/or expression of one or more cell viability/cell function-promoting agents is increased. Without wishing to be limited by theory, the methodologies and compositions disclosed herein may result in the epigenetic modification of one or more cell types that results in at least one characteristic associated with improved cellular function when compared to an otherwise similar cell type not subjected to the compositions and methods disclosed herein. Herein, “epigenetic” refers to the heritable changes in gene activity and expression that occurs without alternation in DNA sequence. Nonlimiting examples of epigenetic modifications include posttranslational modifications such as DNA methylation, chromatin remodeling, and histone modification.

The RC comprises cells that may exhibit alterations in parameters of cellular and/or organismal physiology that result in a perceived and/or quantifiable improvement in the functional state of receiver cells and/or cell types, wherein perceived improvement is defined as semblance to the functional state of the donor cells and/or cell types. It is to be understood that the restored composition comprises cells and is derived from a corresponding receiver cell sample.

In an embodiment, a RC is characterized by the maintenance of the viability state of the cells and/or cell types in the composition as quantified, for example, by a cell vitality assay.

In an alternate embodiment, a RC is characterized by an increase in the viability state of the cells and/or cell types as quantified, for example, by a cell viability assay.

In an embodiment, a RC is characterized by a lack of change in the percentage of hematopoietic stem cells, hematopoietic progenitor cells, mesenchymal stem cells and endothelial progenitor cells, herein termed the “stem cell pool”, compared to the receiver cell sample.

In an alternate embodiment, a RC is characterized by an increase in the stem cell pool in comparison to the receiver cell sample.

In an embodiment, a RC is characterized by cells that exhibit an improvement in cellular immune function in comparison to the corresponding receiver cell sample as quantified, for example, by natural killer cell cytotoxicity assay.

In an embodiment, a RC is characterized by cells that exhibit an improvement in cellular hematopoietic function as quantified, for example, by hematopoietic stem cell clonogenic assay in comparison to the corresponding receiver cell sample.

In an alternate embodiment, a RC is characterized by cells that exhibit an improvement in systematic hematopoietic and immune function of the subject when compared to the corresponding receiver cell sample as quantified, for example, by increased lymphopoiesis, increased ratio of CD4 to CD8 positive T cells, and/or improved immune surveillance. Improved immune surveillance can be determined by decreased incidences of microbial infection and tumor formation in the subject. Improved immune surveillance can also be measured by decreased rate of cancer incidence in the subject. Decreased rates of cancer incidence shall thereby confer to the subject an increased likelihood of prolonged organismal survival.

In an embodiment, a RC is characterized by cells that exhibit a minimization of replicative stress as determined, for example, by telomere length and/or telomerase activity when compared to the corresponding receiver cell sample.

In an embodiment, a RC is characterized by cells that exhibit a decreased expression of senescence-related genes when compared to the corresponding receiver cell sample, wherein senescence-related genes are defined, for example, as the RBGEP, by quantitative polymerase chain reaction.

In an embodiment, a RC is characterized by cells that exhibit a decreased expression of senescence-associated secretory factors when compared to the corresponding receiver cell sample, wherein senescence-associated secretory factors are exemplified in Table 1.

In an embodiment, a RC is characterized by cells that exhibit alterations in the epigenetic signature of the cells when compared to the corresponding receiver cell sample, wherein epigenetic signature is determined, for example, by chromatin immunoprecipitation sequencing (ChIP-Seq).

In an embodiment, a RC is characterized by cells that exhibit an increase in the rate of proteostasis when compared to the corresponding receiver cell sample which may be quantified, for example, by a Cyto-ID® Autogphagy Detection Kit.

In an embodiment, a RC is characterized by cells that exhibit a decrease in cellular oxidative stress when compared to the corresponding receiver cell sample, as quantified for example by the MitoSOX™ Red mitochondrial superoxide indicator kit.

In an embodiment, a RC is characterized by cells that exhibit a decrease in cellular senescence when compared to the corresponding receiver cell sample, as quantified, for example, by the Fluorometric Quantitative Cellular Senescence 3-Gal Assay Kit.

In an embodiment, a RC is characterized by cells that exhibit the maintenance of mesenchymal stem cell function when compared to the corresponding receiver cell sample, wherein mesenchymal stem cell function is quantified, for example, by the colony forming unit-fibroblast (CFU-F) assay and/or ability to undergo lineage-specific differentiation into adipogenic, osteogenic and chondrogenic lineages.

In an alternate embodiment, a RC is characterized by cells that exhibit an increased mesenchymal stem cell function when compared to the corresponding receiver cell sample.

In an embodiment, a RC is characterized by cells that exhibit maintenance of endothelial progenitor function when compared to the corresponding receiver cell sample wherein endothelial progenitor function is quantified, for example, by tube formation assay.

In an alternate embodiment, a RC is characterized by cells that exhibit an increased endothelial progenitor function when compared to the corresponding receiver cell sample.

Herein, cellular restoration occurs following contact of the receiver cell sample with soluble factors and/or particles present in the donor cell sample (e.g., materials that pass through the permeable transwell insert). Consequently, the method of restoration comprises contact of the cell-free soluble factors and/or particles present in the media of a donor cell sample with a receiver cell sample. In some aspects, the donor cell sample is cultured in a suitable media and the media may then be separated from the donor cells to form a cell-free media which is utilized in the restoration of a receiver cell sample. Without wishing to be limited by theory, the soluble factors and/or particles present in the donor cell sample media that pass through the permeable transwell insert may include paracrine factors, microvesicles, exosomes, cellular fragments, and the like Herein paracrine factors refer to signaling molecules which are secreted into the immediate extracellular environment and diffuse over a short distance to a target cell. Microvesicles generally refer to small (e.g., 50 nm to 100 nm) fragments of plasma membrane thought to be shed by a variety of cell types. Exosomes generally refer to secreted extracellular vesicles that may contain biomolecules such as proteins, lipids, and RNA and function in cellular signaling. In some embodiments, the soluble factors and/or particles of the donor cell sample that contact the receiver cell sample comprise extracellular vesicles. Exosomes and microvesicles belong to a broader group of extracellular vesicles (EVs) that represent an important mode of intercellular communication by serving as vehicles for transfer between cells of membrane and cytosolic proteins, lipids, and RNA.

In an embodiment, the quality of the restoring composition can be adjusted by the presence of one or more materials that regulate the release of EVs. For example, the release of one or more EVs may be inhibited by the addition of small molecule inhibitors such as manumycin A. Alternatively, the release of EVs may be promoted, for example, by activation of purinergic receptors with ATP, activation by lipopolysaccharides, plasma membrane depolarization, or increasing intracellular Ca²⁺ concentrations.

It is a contemplated aspect of the present disclosure that cell-free media generated by culturing of the donor cell sample in a suitable media followed by removal of the cells and herein designated the restoring composition, may be further processed to separate individual constituents or groups of constituents based on like characteristics using any suitable methodology (e.g., ethanol precipitation, centrifugation gradients). In an embodiment, the individual constituents of the restoring composition may be analyzed for their ability to affect restoration of a receiver cell sample of the type disclosed herein. The present disclosure further contemplates utilization of one or more isolated constituents, or isolated groups of constituents, in the methodologies for cellular restoration. In an embodiment, cellular restoration of a receiver cell sample of the type disclosed herein is carried out utilizing an EV (e.g., exosome/microvesicle) isolated from a culture media of a donor cell sample. In another embodiment, cellular restoration of a receiver cell sample of the type disclosed herein is carried out utilizing cellular fragments isolated from a culture media of a donor cell sample.

In yet another aspect, the present disclosure contemplates utilization of the restored composition to generate a secondarily restored composition. In particular, the restored composition having the characteristics disclosed herein may be contacted with a third cell sample obtained from a third subject. The third cell sample may be evaluated via the methodologies disclosed herein for evaluation of cell samples (e.g., immunophenotyping). In some embodiments, the third cell sample is obtained from a subject having characteristics (e.g., chronological age, medical condition) disclosed for the first subject. Culturing of the third cell sample in the presence of the restored composition using the methods disclosed herein (e.g., transwell culture) may result in the formation of a secondarily restored composition. It is contemplated that the restoration processes disclosed herein can be propagated indefinitely such that a secondarily restored composition may likewise be utilized to produce a tertiary restored composition utilizing a fourth cell sample obtained from a fourth subject and so on. The extent to which the subsequently restored compositions (e.g., tertiary restored compositions) manifest the characteristics disclosed herein may be evaluated utilizing any suitable methodology (e.g., assay for increased endothelial progenitor function). In some embodiments, subsequently restored compositions (e.g., a secondarily restored composition) when evaluated using assays such as cell viability, the percentage of hematopoietic stem cells, extent of the stem cell pool, cellular hematopoietic and immune function, systematic hematopoietic and immune function, replicative stress, expression of senescence-related genes, level of senescence-associated secretory factors, alterations in epigenetic signature, rate of proteostasis, extent of cellular oxidative stress, extent of cellular senescence, maintenance or increase of mesenchymal stem cell function, maintenance or increase of endothelial progenitor function, or a combination thereof have results that are within from about 10% to about 90% of results obtained from assaying the restored composition, alternatively from about 20% to about 80% or alternatively from about 30% to about 70%.

Within the context of aspects comprising a donor and receiver cell sample, cellular restoration occurs following contact of the receiver cell sample with soluble factors and/or particles present in the donor cell sample (e.g., materials that pass through the permeable transwell insert). Likewise utilizing a first and second cell sample; the first cell sample is considered the receiver sample while the second cell sample is considered the donor sample. Once a restored composition is formed it can function as the donor sample in subsequent restorations.

Consequently, the method of restoration comprises contact of the cell-free soluble factors and/or particles present in the media of a donor cell sample with a receiver cell sample. In some aspects, the donor cell sample is cultured in a suitable media and the media may then be separated from the donor cells to form a cell-free media which is utilized in the restoration of a receiver cell sample. Without wishing to be limited by theory, the soluble factors and/or particles present in the donor cell sample media that pass through the permeable transwell insert may include paracrine factors, microvesicles, exosomes, cellular fragments, and the like Herein paracrine factors refer to signaling molecules which are secreted into the immediate extracellular environment and diffuse over a short distance to a target cell. Microvesicles generally refer to small (e.g., 50 nm to 100 nm) fragments of plasma membrane thought to be shed by a variety of cell types. Exosomes generally refer to secreted extracellular vesicles that may contain biomolecules such as proteins, lipids, and RNA and function in cellular signaling. In some embodiments, the soluble factors and/or particles of the donor cell sample that contact the receiver cell sample comprise extracellular vesicles. Exosomes and microvesicles belong to a broader group of extracellular vesicles (EVs) that represent an important mode of intercellular communication by serving as vehicles for transfer between cells of membrane and cytosolic proteins, lipids, and RNA. In some embodiments, disclosed herein are microRNAs within exosomes of young blood that can restore function to the aging lymphohematopoietic system as described herein and compositions comprising same, and methods of making same.

In some embodiments, the quality of the restoring composition can be adjusted by the presence of one or more materials that regulate the release of EVs. For example, the release of one or more EVs may be inhibited by the addition of small molecule inhibitors such as manumycin A. Alternatively, the release of EVs may be promoted, for example, by activation of purinergic receptors with ATP, activation by lipopolysaccharides, plasma membrane depolarization, or increasing intracellular Ca²⁺ concentrations.

In some embodiments, the RC may be formulated for administration to a subject in need thereof. In some embodiments, the subject is the receiver subject. For example, the RC may be a component of a formulation that is administered to the receiver subject to improve the receiver subject's general health. Such improvements may be identified by quantitative evaluation of one or more physiological or psychological parameters of the subject. In the alternative, such improvements may be identified by the qualitative evaluations of one or more physiological or psychological parameters of the subject. In an embodiment, the receiver subject is prophylactically administered the RC.

The RC may be administered to a subject (e.g., receiver subject) via any suitable methodology. In some embodiments, the methodologies disclosed herein comprise systemic administration of the RC to the subject. For example, the RC may be administered systemically in dosage unit formulations containing conventional nontoxic pharmaceutically acceptable carriers, adjuvants, and vehicles as desired. In a specific embodiment, administration of the RC may be by intravenous injection, endobronchial administration, intraarterial injection, intramuscular injection, intracardiac injection, subcutaneous injection, intraperitoneal injection, intraperitoneal infusion, transdermal diffusion, transmucosal diffusion, intracranial, intrathecal, or combinations thereof. A means of administering the RC may include, but is not limited to, infusion. Systemically may also include, for example, by a pump, by an intravenous line, or by bolus injection. Bolus injection can include subcutaneous, intramuscular, or intraperitoneal routes.

In some embodiments, one or more RNAi(s) and/or compounds as disclosed elsewhere herein are administered locally or systemically to a subject in need thereof. In some aspects, the appropriate route of administration of one or more RNAi(s) and/or compounds as disclosed elsewhere herein is selected based upon various factors such as the type of medical condition, the underlying cause, the severity of the condition, etc. In some embodiments, suitable routes of administration include, but are not limited to, oral, intravenous, rectal, aerosol, parenteral, ophthalmic, pulmonary, transmucosal, transdermal, vaginal, optic, nasal, and topical administration. In some embodiments, parenteral delivery includes but is not limited to intramuscular, subcutaneous, intravenous, intramedullary injections, as well as intrathecal, direct intraventricular, intraperitoneal, intralymphatic, and intranasal injections.

In some embodiments, one or more RNAi(s) and/or compounds as disclosed elsewhere herein is formulated for oral administration. In some embodiments, one or more RNAi(s) and/or compounds as disclosed elsewhere herein are formulated (e.g., in a pharmaceutical composition) by combining the active agent or agents with, e.g., pharmaceutically acceptable carriers or excipients. In some embodiments, one or more RNAi(s) and/or compounds as disclosed elsewhere herein, such as a cell-free composition, is formulated in oral dosage forms that include tablets, powders, pills, dragees, capsules, liquids, gels, syrups, elixirs, slurries, suspensions and the like.

In some embodiments, one or more RNAi(s) and/or compounds as disclosed elsewhere herein are administered topically. Topical administration may be particularly useful for treatment or prevention of scarring resulting from injury or surgery. In some embodiments, one or more RNAi(s) and/or compounds as disclosed elsewhere herein may be formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In some embodiments, one or more RNAi(s) and/or compounds as disclosed elsewhere herein is formulated for transdermal administration. In some embodiments, transdermal formulations may employ transdermal delivery devices and transdermal delivery patches and can be lipophilic emulsions or buffered, aqueous solutions, dissolved and/or dispersed in a polymer or an adhesive. In some embodiments, such patches are constructed for continuous, pulsatile, or on demand delivery of pharmaceutical agents. In some embodiments, the transdermal delivery of the one or more RNAi(s) and/or compounds as disclosed elsewhere herein is accomplished by means of iontophoretic patches and the like. In some embodiments, transdermal patches provide controlled delivery. In some embodiments, the rate of absorption is slowed by using rate-controlling membranes or by trapping the one or more RNAi(s) and/or compounds as disclosed elsewhere herein within a polymer matrix or gel. In some embodiments, absorption enhancers are used to increase absorption. Absorption enhancers or carriers may include absorbable pharmaceutically acceptable solvents that assist passage through the skin. In some embodiments, transdermal devices are in the form of a bandage comprising a backing member, a reservoir containing an active agent optionally with carriers, optionally a rate controlling barrier to deliver the compound to the skin of the host at a controlled and predetermined rate over a prolonged period of time, and means to secure the device to the skin.

In some embodiments, the active agent or agents are formulated for administration by inhalation. Various forms suitable for administration by inhalation include, but are not limited to, aerosols, mists or powders. In some embodiments, the active agent or agents are conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide, and/or other suitable gases). In some embodiments, the dosage unit of a pressurized aerosol is determined by providing a valve to deliver a metered amount. In some embodiments, capsules and cartridges of, such as, by way of example only, gelatins for use in an inhaler or insufflator are formulated containing a powder mix of one or more RNAi(s) and/or compounds as disclosed elsewhere herein and a suitable powder base such as lactose or starch.

As addressed above, other routes of administration, useful for the treatment of particular conditions or delivery to particular cells, tissues, organs, etc. are contemplated. A means of administering the one or more RNAi(s), compounds, or target cells as disclosed elsewhere herein may include, but are not limited to, infusion. Systemically may also include, for example, by a pump, by an intravenous line, or by bolus injection. In some embodiments, bolus injection can include subcutaneous, intramuscular, or intraperitoneal routes.

The phrases “systemic administration” or “administered systemically,” as used herein, mean the administration of one or more RNAi(s), compounds, or cells as disclosed elsewhere herein, a composition, drug, or other material such that it enters the subject's system and, thus, is subject to metabolism and other like processes, for example, subcutaneous administration.

In some embodiments, the one or more RNAi(s), compounds, and/or cells as disclosed elsewhere herein is locally administered by means such as, but not limited to, injection, implantation, grafting, or epicutaneous. For example, the active agent or agents may be administered proximal to a wound site on the subject and functions to ameliorate the symptoms associated with the wound or increase the rate of wound-healing. Administration of the one or more RNAi(s), compounds, and/or cells as disclosed elsewhere herein may be conducted in any manner compatible with the compositions disclosed herein and to meet one or more user and/or process goals.

In some embodiments, the one or more RNAi(s), compounds, or target cells as disclosed elsewhere herein may be formulated for administration to a subject in order to improve the subject's general health. Such improvements may be identified by quantitative evaluation of one or more physiological or psychological parameters of the subject. In some embodiments, such improvements may be identified by the qualitative evaluations of one or more physiological or psychological parameters of the subject.

In some embodiments, the patient (e.g., receiver subject) is administered the one or more RNAi(s), compounds, or target cells as disclosed elsewhere herein as a component of a therapeutic procedure designed to ameliorate the effects of a medical condition. In some embodiments, the the one or more RNAi(s), compounds, or target cells as disclosed elsewhere herein, present in a therapeutically effective amount, may function as an active agent in a pharmaceutical composition.

In some embodiments, a subject being administered one or more RNAi(s), compounds, or target cells as disclosed elsewhere as disclosed elsewhere herein may be administered additional active agents as considered beneficial for the treatment of the medical condition. Such additional active agents may be administered prior to, concurrent with, or subsequent to the administration of the one or more RNAi(s), compounds, or target cells as disclosed elsewhere. Such additional active agents may be administered by the same route or by a different route, including any route disclosed herein for another active agent. Examples of additional active agents include but are not limited to: (a) antimicrobials, (b) steroids (e.g., hydrocortisone, triamcinolone); (c) pain medications (e.g., aspirin, an NSAID, and a local anesthetic); (d) anti-inflammatory agents; (e) growth factors; (f) cytokines; (g) hormones; or (h) combinations thereof. In some embodiments, additional active agents may also be present in a therapeutically effective amount. In some embodiments, the therapeutically effective amount is lower than would be required if the additional active agents were administered without the one or more RNAi(s), compounds, or target cells.

Examples of additional active agents for administration with one or more RNAi(s), compounds, or target cells include, but are not limited to, anesthetics, hypnotics, sedatives and sleep inducers, antipsychotics, antidepressants, antiallergics, antianginals, antiarthritics, antiasthmatics, antidiabetics, antidiarrheal drugs, anticonvulsants, antigout drugs, antihistamines, antipruritics, emetics, antiemetics, antispasmodics, appetite suppressants, neuroactive substances, neurotransmitter agonists, antagonists, receptor blockers and reuptake modulators, beta-adrenergic blockers, calcium channel blockers, disulfuram and disulfuram-like drugs, muscle relaxants, analgesics, antipyretics, stimulants, anticholinesterase agents, parasympathomimetic agents, hormones, anticoagulants, antithrombotics, thrombolytics, immunoglobulins, immunosuppressants, hormone agonists/antagonists, vitamins, antimicrobial agents, antineoplastics, antacids, digestants, laxatives, cathartics, antiseptics, diuretics, disinfectants, fungicides, ectoparasiticides, antiparasitics, heavy metals, heavy metal antagonists, chelating agents, gases and vapors, alkaloids, salts, ions, autacoids, digitalis, cardiac glycosides, antiarrhythmics, antihypertensives, vasodilators, vasoconstrictors, antimuscarinics, ganglionic stimulating agents, ganglionic blocking agents, neuromuscular blocking agents, adrenergic nerve inhibitors, anti-oxidants, vitamins, cosmetics, anti-inflammatories, wound care products, antithrombogenic agents, antitumoral agents, antiangiogenic agents, anesthetics, antigenic agents, wound healing agents, plant extracts, growth factors, emollients, humectants, rejection/anti-rejection drugs, spermicides, conditioners, antibacterial agents, antifungal agents, antiviral agents, antibiotics, tranquilizers, cholesterol-reducing drugs, antitussives, histamine-blocking drugs, monoamine oxidase inhibitor, or combinations thereof.

In some embodiments, specific compounds suitable for use with the one or more RNAi(s), compounds, or target cells include but are not limited to silver sulfadiazine, Nystatin, Nystatin/triamcinolone, Bacitracin, nitrofurazone, nitrofurantoin, a polymyxin (e.g., Colistin, Surfactin, Polymyxin E, and Polymyxin B), doxycycline, antimicrobial peptides (e.g., natural and synthetic origin), NEOSPORIN® (i.e., Bacitracin, Polymyxin B, and Neomycin), POLYSPORIN® (i.e., Bacitracin and Polymyxin B). Additional antimicrobials include topical antimicrobials (i.e., antiseptics), examples of which include silver salts, iodine, benzalkonium chloride, alcohol, hydrogen peroxide, chlorhexidine, acetaminophen; Alfentanil Hydrochloride; Aminobenzoate Potassium; Aminobenzoate Sodium; Anidoxime; Anileridine; Anileridine Hydrochloride; Anilopam Hydrochloride; Anirolac; Antipyrine; Aspirin; Benoxaprofen; Benzydamine Hydrochloride; Bicifadine Hydrochloride; Brifentanil Hydrochloride; Bromadoline Maleate; Bromfenac Sodium; Buprenorphine Hydrochloride; Butacetin; Butixirate; Butorphanol; Butorphanol Tartrate; Carbamazepine; Carbaspirin Calcium; Carbiphene Hydrochloride; Carfentanil Citrate; Ciprefadol Succinate; Ciramadol; Ciramadol Hydrochloride; Clonixeril; Clonixin; Codeine; Codeine Phosphate; Codeine Sulfate; Conorphone Hydrochloride; Cyclazocine; Dexoxadrol Hydrochloride; Dexpemedolac; Dezocine; Diflunisal; Dihydrocodeine Bitartrate; Dimefadane; Dipyrone; Doxpicomine Hydrochloride; Drinidene; Enadoline Hydrochloride; Epirizole; Ergotamine Tartrate; Ethoxazene Hydrochloride; Etofenamate; Eugenol; Fenoprofen; Fenoprofen Calcium; Fentanyl Citrate; Floctafenine; Flufenisal; Flunixin; Flunixin Meglumine; Flupirtine Maleate; Fluproquazone; Fluradoline Hydrochloride; Flurbiprofen; Hydromorphone Hydrochloride; Ibufenac; Indoprofen; Ketazocine; Ketorfanol; Ketorolac Tromethamine; Letimide Hydrochloride; Levomethadyl Acetate; Levomethadyl Acetate Hydrochloride; Levonantradol Hydrochloride; Levorphanol Tartrate; Lofemizole Hydrochloride; Lofentanil Oxalate; Lorcinadol; Lomoxicam; Magnesium Salicylate; Mefenamic Acid; Menabitan Hydrochloride; Meperidine Hydrochloride; Meptazinol Hydrochloride; Methadone Hydrochloride; Methadyl Acetate; Methopholine; Methotrimeprazine; Metkephamid Acetate; Mimbane Hydrochloride; Mirfentanil Hydrochloride; Molinazone; Morphine Sulfate; Moxazocine; Nabitan Hydrochloride; Nalbuphine Hydrochloride; Nalmexone Hydrochloride; Namoxyrate; Nantradol Hydrochloride; Naproxen; Naproxen Sodium; Naproxol; Nefopam Hydrochloride; Nexeridine Hydrochloride; Noracymethadol Hydrochloride; Ocfentanil Hydrochloride; Octazamide; Olvanil; Oxetorone Fumarate; Oxycodone; Oxycodone Hydrochloride; Oxycodone Terephthalate; Oxymorphone Hydrochloride; Pemedolac; Pentamorphone; Pentazocine; Pentazocine Hydrochloride; Pentazocine Lactate; Phenazopyridine Hydrochloride; Phenyramidol Hydrochloride; Picenadol Hydrochloride; Pinadoline; Pirfenidone; Piroxicam Olamine; Pravadoline Maleate; Prodilidine Hydrochloride; Profadol Hydrochloride; Propirarn Fumarate; Propoxyphene Hydrochloride; Propoxyphene Napsylate; Proxazole; Proxazole Citrate; Proxorphan Tartrate; Pyrroliphene Hydrochloride; Remifentanil Hydrochloride; Salcolex; Salethamide Maleate; Salicylamide; Salicylate Meglumine; Salsalate; Sodium Salicylate; Spiradoline Mesylate; Sufentanil; Sufentanil Citrate; Talmetacin; Talniflumate; Talosalate; Tazadolene Succinate; Tebufelone; Tetrydamine; Tifurac Sodium; Tilidine Hydrochloride; Tiopinac; Tonazocine Mesylate; Tramadol Hydrochloride; Trefentanil Hydrochloride; Trolamine; Veradoline Hydrochloride; Verilopam Hydrochloride; Volazocine; Xorphanol Mesylate; Xylazine Hydrochloride; Zenazocine Mesylate; Zomepirac Sodium; Zucapsaicin, Aflyzosin Hydrochloride; Alipamide; Althiazide; Amiquinsin Hydrochloride; Amlodipine Besylate; Amlodipine Maleate; Anaritide Acetate; Atiprosin Maleate; Belfosdil; Bemitradine; Bendacalol Mesylate; Bendroflumethiazide; Benzthiazide; Betaxolol Hydrochloride; Bethanidine Sulfate; Bevantolol Hydrochloride; Biclodil Hydrochloride; Bisoprolol; Bisoprolol Fumarate; Bucindolol Hydrochloride; Bupicomide; Buthiazide: Candoxatril; Candoxatrilat; Captopril; Carvedilol; Ceronapril; Chlorothiazide Sodium; Cicletanine; Cilazapril; Clonidine; Clonidine Hydrochloride; Clopamide; Cyclopenthiazide; Cyclothiazide; Darodipine; Debrisoquin Sulfate; Delapril Hydrochloride; Diapamide; Diazoxide; Dilevalol Hydrochloride; Diltiazem Malate; Ditekiren; Doxazosin Mesylate; Eeadotril; Enalapril Maleate; Enalaprilat; Enalkiren; Endralazine Mesylate; Epithiazide; Eprosartan; Eprosartan Mesylate; Fenoldopam Mesylate; Flavodilol Maleate; Flordipine; Flosequinan; Fosinopril Sodium; Fosinoprilat; Guanabenz; Guanabenz Acetate; Guanacline Sulfate; Guanadrel Sulfate; Guancydine; Guanethidine Monosulfate; Guanethidine Sulfate; Guanfacine Hydrochloride; Guanisoquin Sulfate; Guanoclor Sulfate; Guanoctine Hydrochloride; Guanoxabenz; Guanoxan Sulfate; Guanoxyfen Sulfate; Hydralazine Hydrochloride; Hydralazine Polistirex; Hydroflumethiazide; Indacrinone; Indapamide; Indolaprif Hydrochloride; Indoramin; Indoramin Hydrochloride; Indorenate Hydrochloride; Lacidipine; Leniquinsin; Levcromakalim; Lisinopril; Lofexidine Hydrochloride; Losartan Potassium; Losulazine Hydrochloride; Mebutamate; Mecamylamine Hydrochloride; Medroxalol; Medroxalol Hydrochloride; Methalthiazide; Methyclothiazide; Methyldopa; Methyldopate Hydrochloride; Metipranolol; Metolazone; Metoprolol Fumarate; Metoprolol Succinate; Metyrosine; Minoxidil; Monatepil Maleate; Muzolimine; Nebivolol; Nitrendipine; Ofornine; Pargyline Hydrochloride; Pazoxide; Pelanserin Hydrochloride; Perindopril Erbumine; Phenoxybenzamine Hydrochloride; Pinacidil; Pivopril; Polythiazide; Prazosin Hydrochloride; Primidolol; Prizidilol Hydrochloride; Quinapril Hydrochloride; Quinaprilat; Quinazosin Hydrochloride; Quinelorane Hydrochloride; Quinpirole Hydrochloride; Quinuclium Bromide; Ramipril; Rauwolfia Serpentina; Reserpine; Saprisartan Potassium; Saralasin Acetate; Sodium Nitroprusside; Sulfinalol Hydrochloride; Tasosartan; Teludipine Hydrochloride; Temocapril Hydrochloride; Terazosin Hydrochloride; Terlakiren; Tiamenidine; Tiamenidine Hydrochloride; Tierynafen; Tinabinol; Tiodazosin; Tipentosin Hydrochloride; Trichlormethiazide; Trimazosin Hydrochloride; Trimethaphan Camsylate; Trimoxamine Hydrochloride; Tripamide; Xipamide; Zankiren Hydrochloride; Zofenoprilat Arginine, Alclofenac; Alclometasone Dipropionate; Algestone Acetonide; Alpha Amylase; Ameinafal; Ameinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium or combinations thereof.

Although the compositions provided herein are suitable for administration to humans, such compositions are generally suitable for administration to animals of all sorts. Modification of compositions suitable for administration to humans of the type disclosed herein (i.e., one or more RNAi(s), compounds, or target cells) in order to render the compositions suitable for administration to various animals can be accomplished by the ordinarily skilled veterinary pharmacologist, with the benefit of this disclosure, who can design and perform such modifications with routine, if any, experimentation. Subjects to which administration of the pharmaceutical compositions of this disclosure is contemplated include, but are not limited to, humans and other primates; mammals including commercially relevant mammals such as cattle, pigs, horses, and sheep; companion animals such as cats and dogs; and birds including commercially relevant birds such as chickens, ducks, geese, and turkeys.

In some embodiments, the composition may contain additional ingredients as suitable for the formulation of a pharmaceutical composition. As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents (e.g., water, dimethylsulfoxide, combinations thereof, or other solvents); oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials.

In some embodiments, the composition may be administered systemically in dosage unit formulations containing conventional nontoxic pharmaceutically acceptable carriers, adjuvants, and vehicles as desired, or may be locally administered by means such as, but not limited to, injection, implantation, or grafting. In some embodiments, administration may be by intravenous injection, endobronchial administration, intraarterial injection, intramuscular injection, intracardiac injection, subcutaneous injection, intraperitoneal injection, intraperitoneal infusion, transdermal diffusion, transmucosal diffusion, intracranial, intrathecal, or combinations thereof. In some embodiments, the compositions are administered by infusion. Systemic administration may also include, for example, by a pump, by an intravenous line, or by bolus injection. Bolus injection can include subcutaneous, intramuscular, or intraperitoneal routes.

In some embodiments, the one or more RNAi(s), compounds, or target cells is formulated for topical administration into forms such as creams, lotions, serums, powders, ointments, or drops. A formulation of the one or more RNAi(s), compounds, or target cells for topical administration may also contain pharmaceutically acceptable carriers, moisturizers, oils, fats, waxes, surfactants, thickening agents, antioxidants, viscosity stabilizers, chelating agents, buffers, preservatives, perfumes, dyestuffs, lower alkanols, humectants, emollients, dispersants, sunscreens such as radiation blocking compounds or UV-blockers, antibacterials, antifungals, disinfectants, vitamins, antibiotics, anti-acne agents, as well as other suitable materials that do not have a significant adverse effect on the activity of the topical composition or combinations thereof.

Nonlimiting exemplary pharmaceutically acceptable carriers that may be used in the compositions for topical administration or other forms of administration include water, mixtures of water and water-miscible solvents (e.g., lower alkanols, vegetable oils, DMSO, etc.), and water-soluble ophthalmologically acceptable non-toxic polymers (for example, cellulose derivatives such as methylcellulose), glycerin, propylene glycol, methylparaben, alginates, glyceryl stearate, PEG-100 stearate, cetyl alcohol, propylparaben, butylparaben, sorbitols, polyethoxylated anhydrosorbitol monostearate (TWEEN®), white petrolatum (VASELINE®), triethanolamine, emu oil, aloe vera extract, lanolin, cocoa butter, LIPODERM® base, and the like or combinations thereof. In some embodiments, the one or more RNAi(s), compounds, or target cells formulated for topical administration may be applied to one or more areas of the skin including the face, hands, and neck.

In some embodiments, the methodologies disclosed herein result in therapies that are prophylactic, palliative, curative, or combinations thereof. Methodologies and compositions of the type disclosed herein may be utilized in the treatment of a wide variety of medical conditions related to decreases in cellular function and viability such as age-related medical conditions that include neurological disorders; autoimmune diseases; infectious disease; cancer and disorders associated with radiation overexposure (chronic or acute).

It is contemplated the methodologies and compositions disclosed herein may result in an increased expression of genes associated with improved cellular health with a concomitant decrease in the expression of genes associated with adverse cellular events. In some embodiments, the methodologies and compositions disclosed herein result in an increased expression of genes associated with beneficial cellular events.

According to another aspect of the disclosure, kits are provided. Kits, according to the present disclosure, include package(s) or containers comprising the compositions disclosed herein (e.g., RC, cell-free culture media) and may include defined culture medium and cell culture medium supplement. The kit may further include an instruction letter or package-associated instruction for the treatment and/or prophylaxis of a medical condition. The phrase “package” means any vessel containing the compositions (including stem cells, media, and/or media supplement) presented herein. For example, the package can be a box or wrapping. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. The kit can also contain items that are not contained within the package but are attached to the outside of the package, for example, pipettes. Kits may optionally contain instructions for administering compositions of the present disclosure to a subject having a condition in need of treatment. Kits may also comprise instructions for approved uses of compounds herein by regulatory agencies, such as the United States Food and Drug Administration. Kits may optionally contain labeling or product inserts for the present compositions. The package(s) and/or any product insert(s) may themselves be approved by regulatory agencies. The kits can include compounds in the solid phase or in a liquid phase (such as buffers provided) in a package. The kits also can include buffers for preparing solutions for conducting the methods, and pipettes for transferring liquids from one container to another. The kit may optionally also contain one or more other compounds for use in combination therapies as described herein. In certain embodiments, the package(s) is a container for intravenous administration.

In an embodiment, a subject having undergone a restoration method of the type disclosed herein may be subsequently monitored for some time period. Monitoring of the subject may comprise qualitative and quantitative evaluations of the subject's general health and/or medical condition. In some embodiments, a subject may be subjected to a plurality of cellular restoration methods of the type disclosed herein. For example, a receiver subject having undergone a cellular restoration method of the type disclosed herein may display quantitative and/or qualitative improvements in the subject's general health and/or medical condition for some time period. Subsequently, the receiver subject may experience some decline in their general health and/or medical condition and another cellular restoration process may be carried out. The cellular restoration process may involve obtaining a donor cell sample and/or receiver cell sample utilizing the methodologies disclosed herein, performing cellular restoration of the receiver cell sample and administering the restored cell sample to the receiver subject. Alternatively, the receiver subject may be administered at least a portion of the restored cell sample remaining from a prior cellular restoration process.

In some embodiments, evaluations of the subject comprise determinations based on analyses disclosed herein (e.g., natural killer assay, telomere length, gene and protein biomarker arrays). In such embodiments, the subject may provide a receiver cell sample and the quality of the sample evaluated as disclosed herein. In some embodiments, the receiver cell sample quality value at some point post-restoration may be compared to the receiver cell sample quality value pre-restoration and this information utilized to assess whether additional treatment is needed. For example, a subject having a receiver cell sample pre-restoration quality value of 5 may have a receiver cell sample post-restoration quality value of 9 for a time period of up to about 1 year subsequent to the restoration process. The subject's post-restoration receiver cell sample quality value after 1.5 years may have decreased to 7 while after 3 years the value may be 5. In such instances, the subject may be administered another RC.

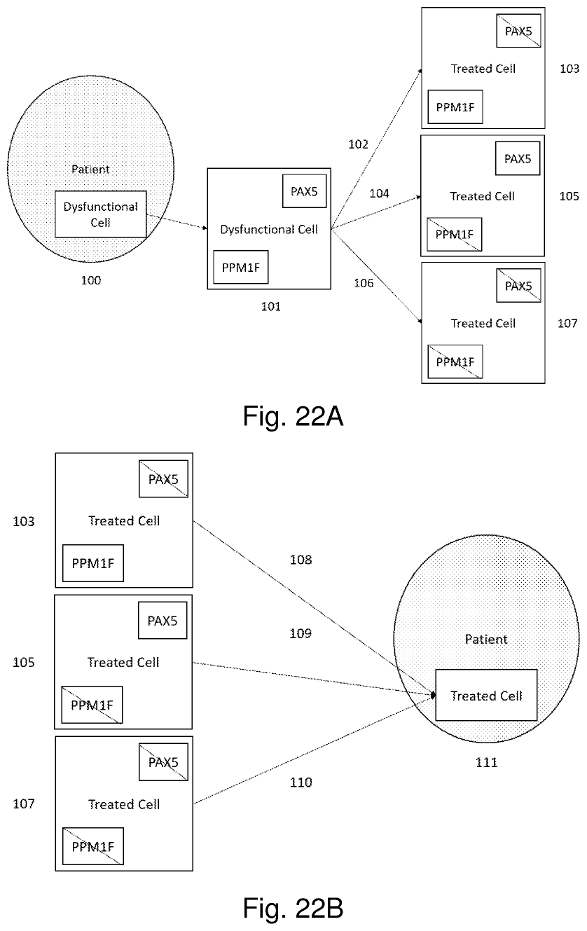

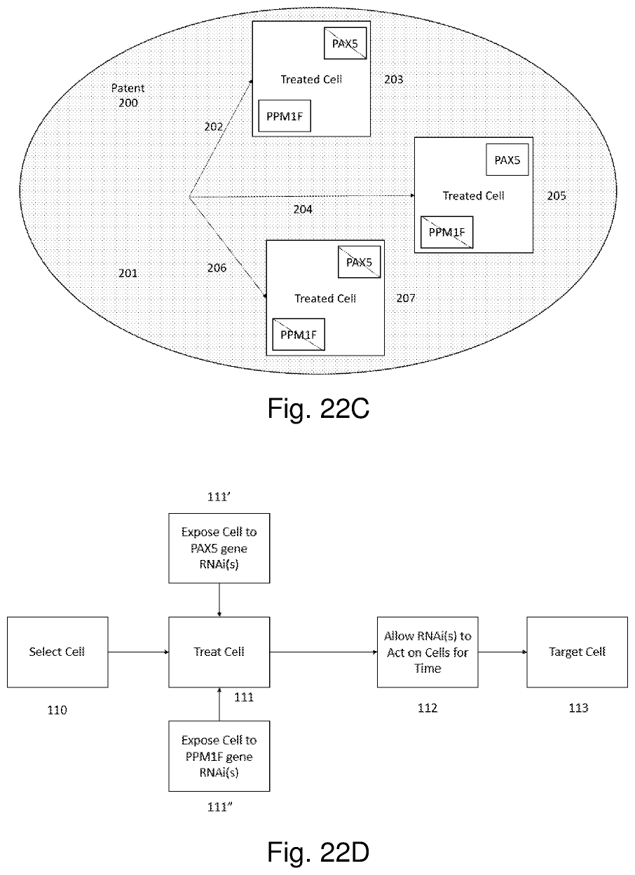

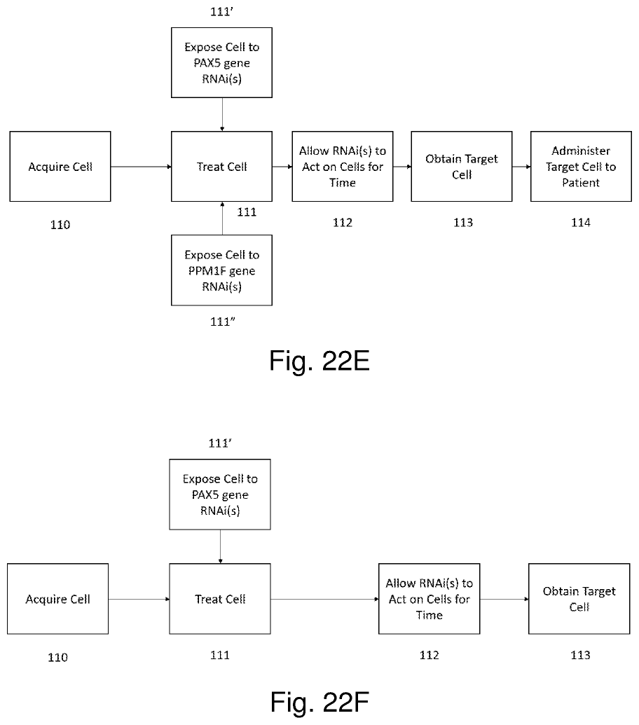

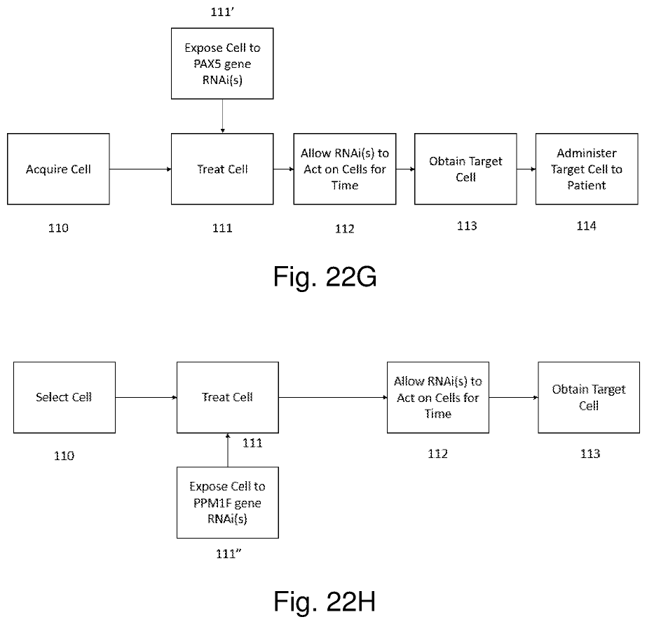

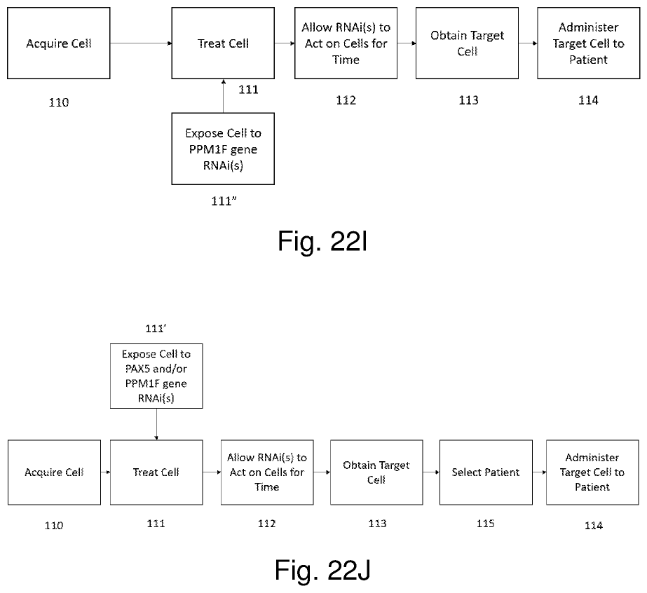

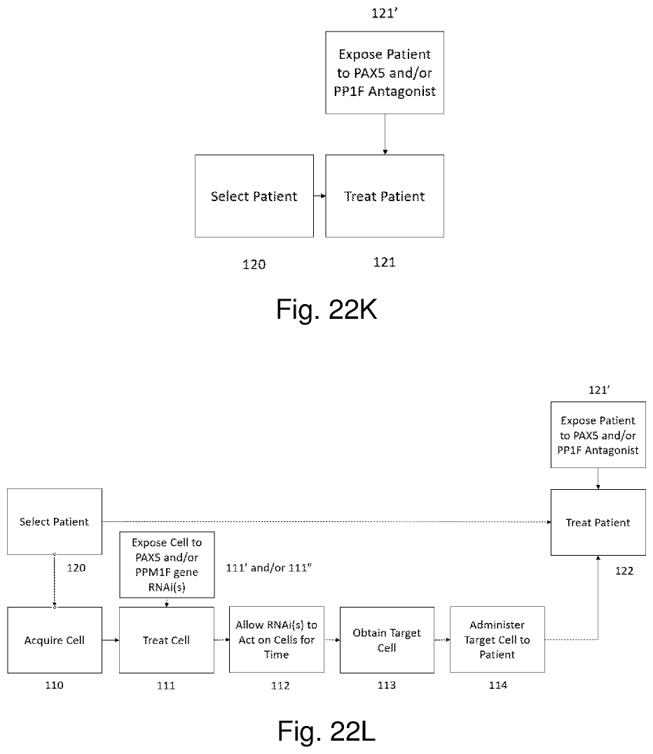

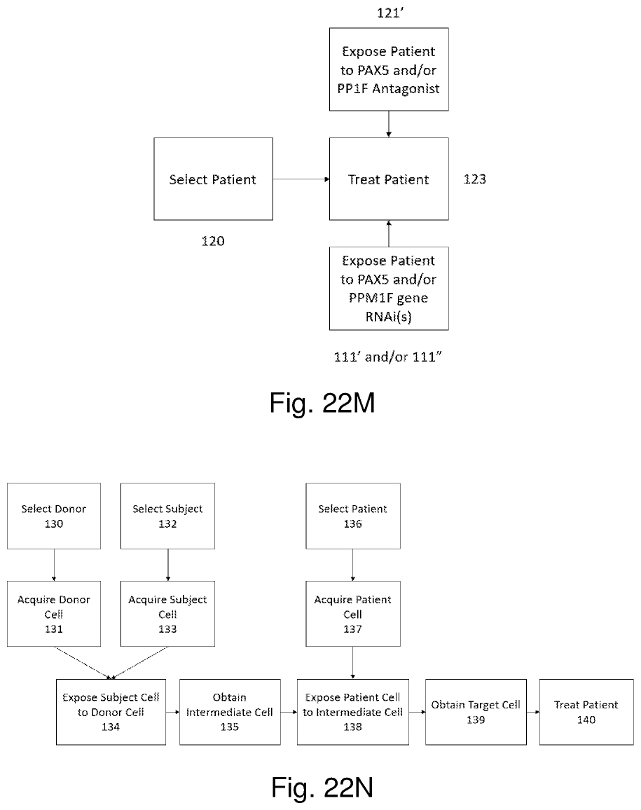

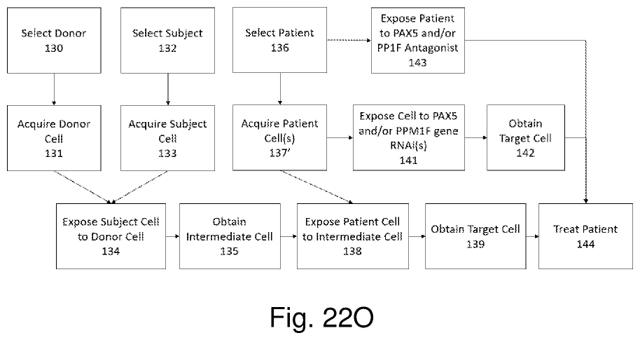

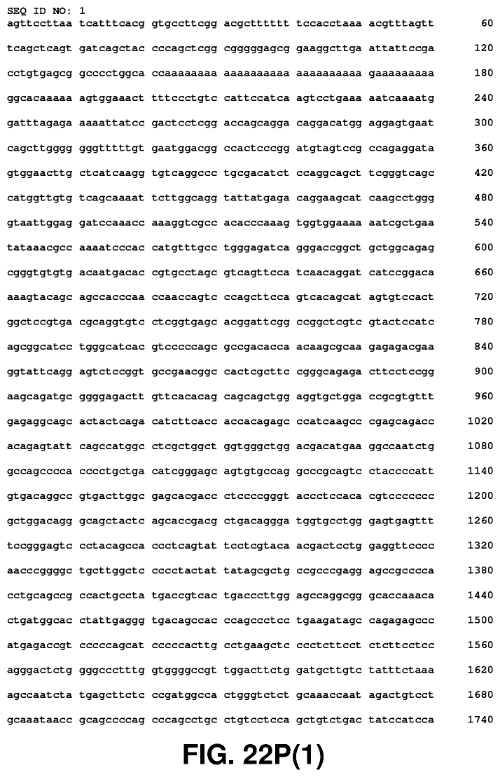

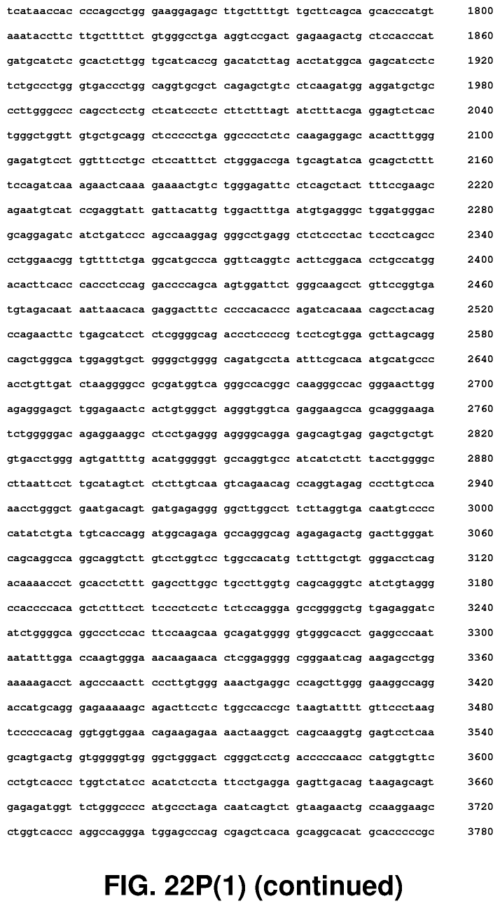

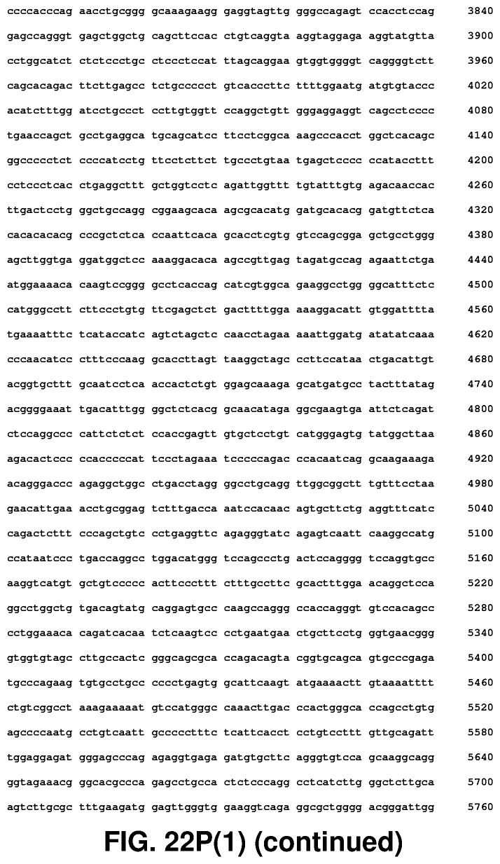

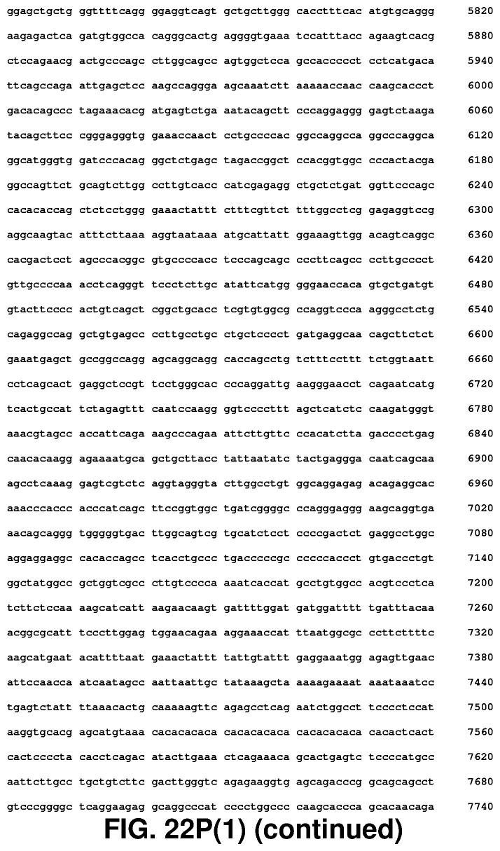

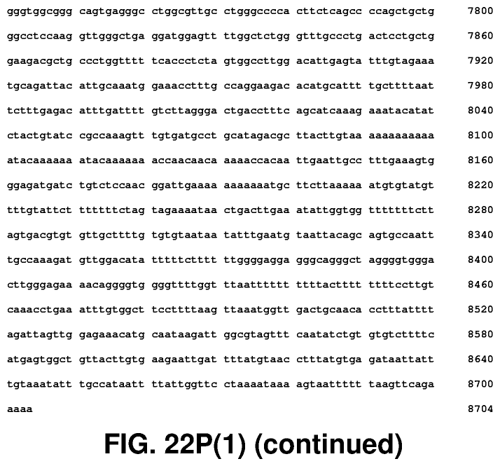

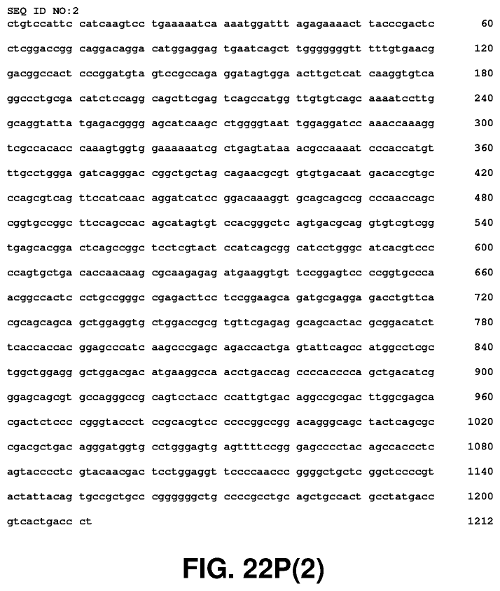

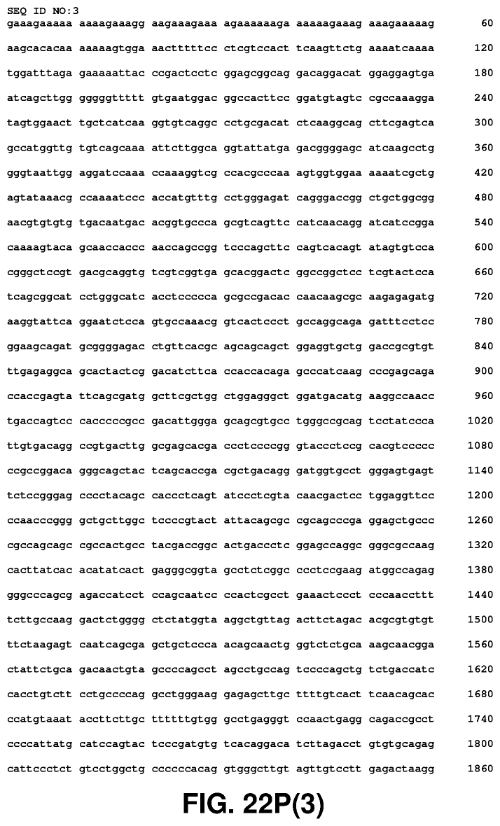

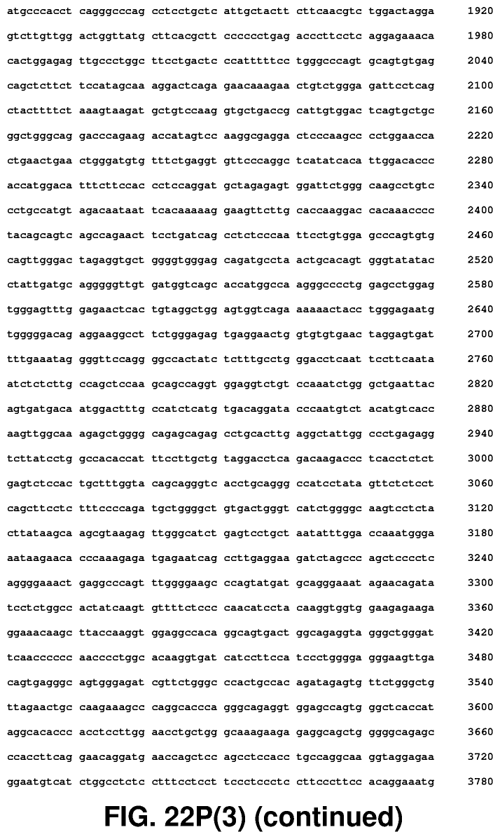

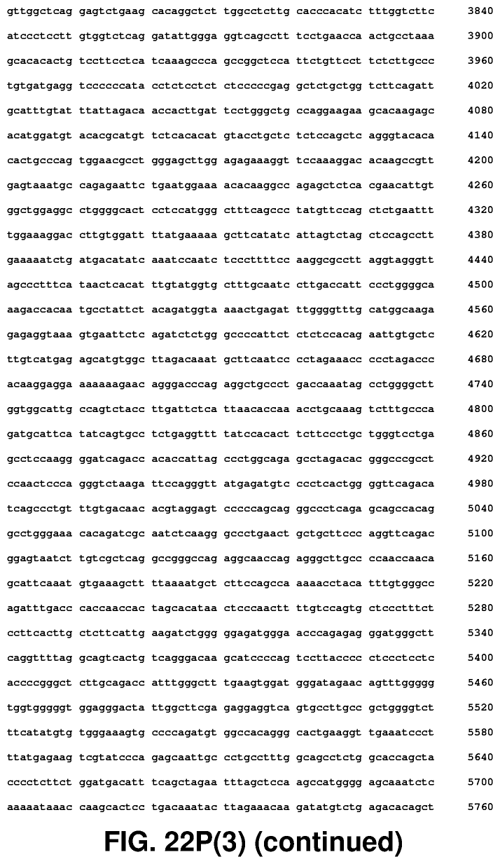

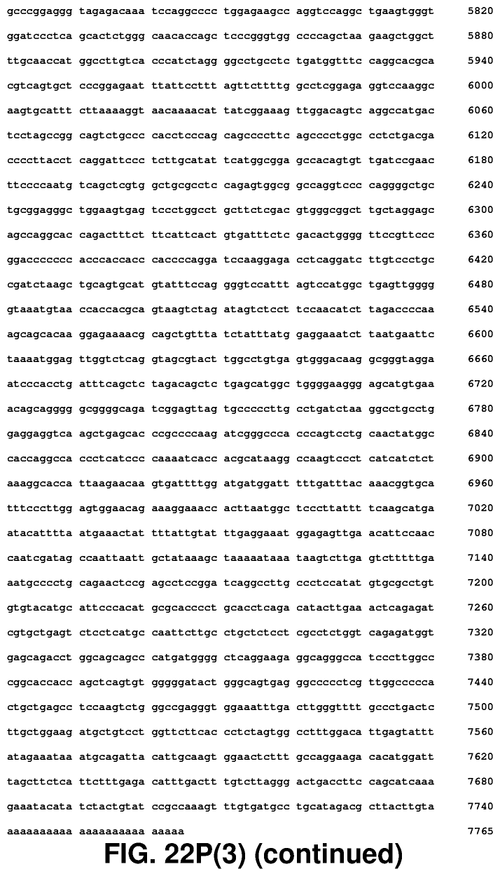

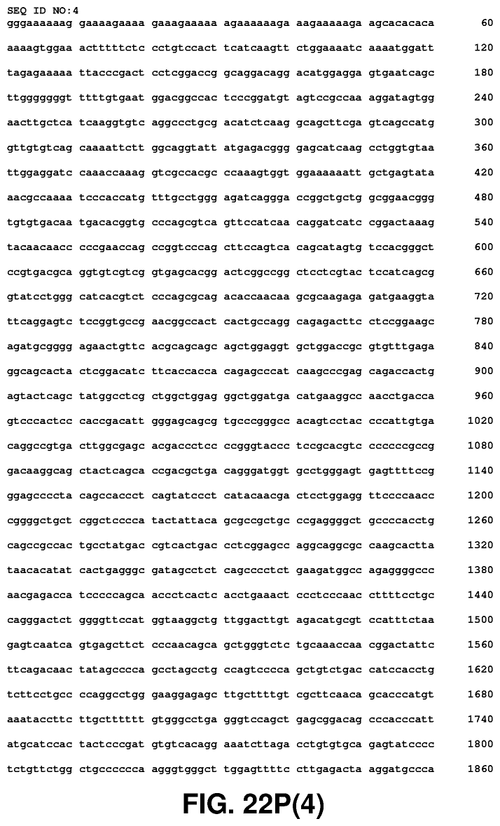

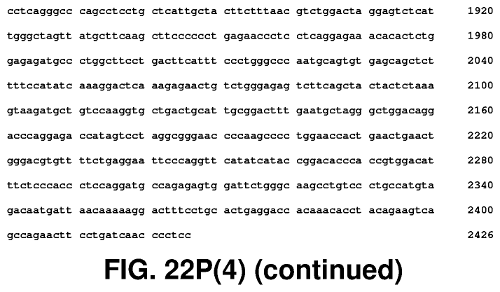

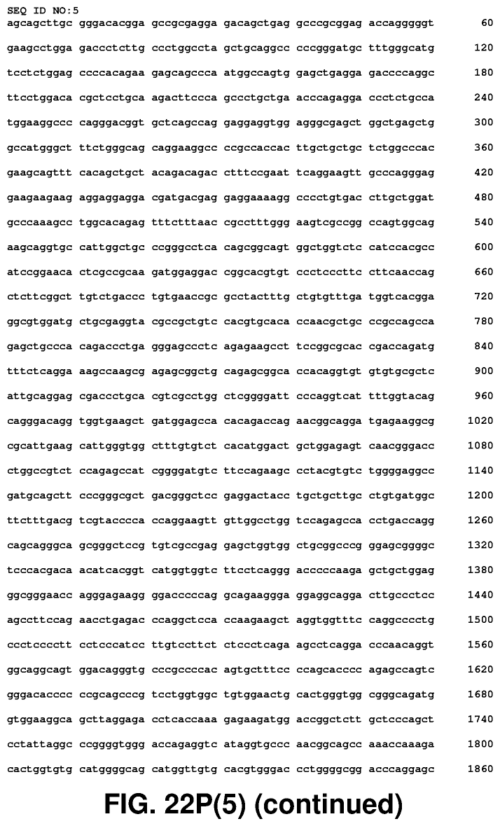

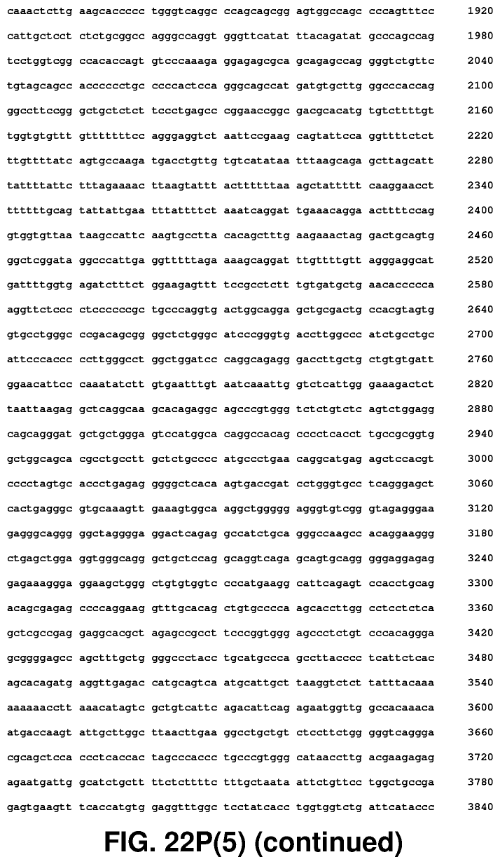

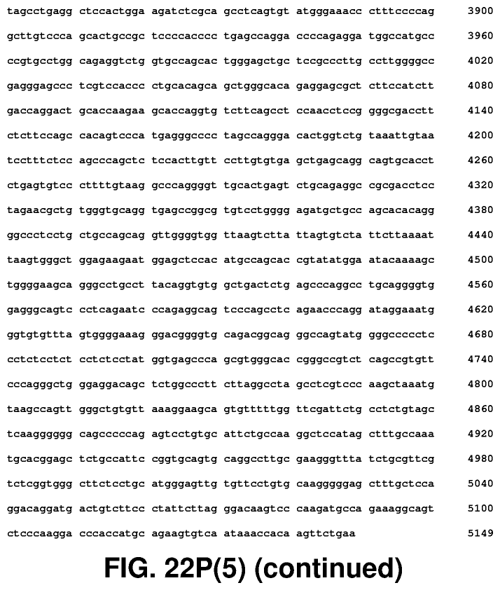

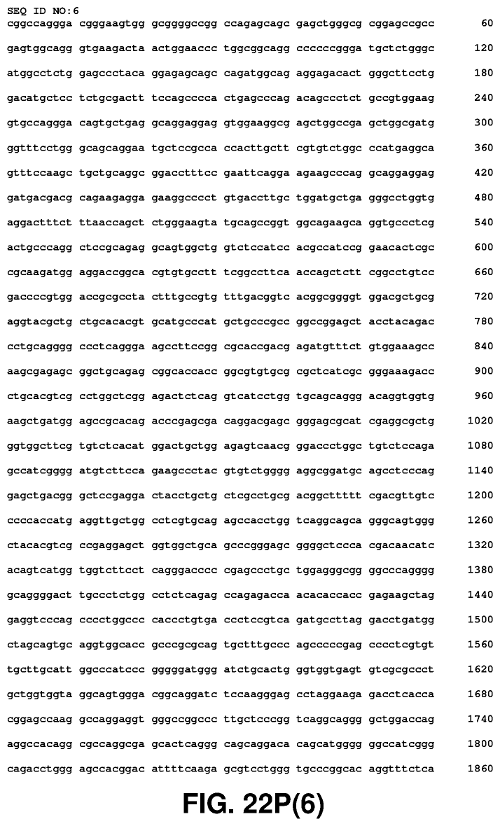

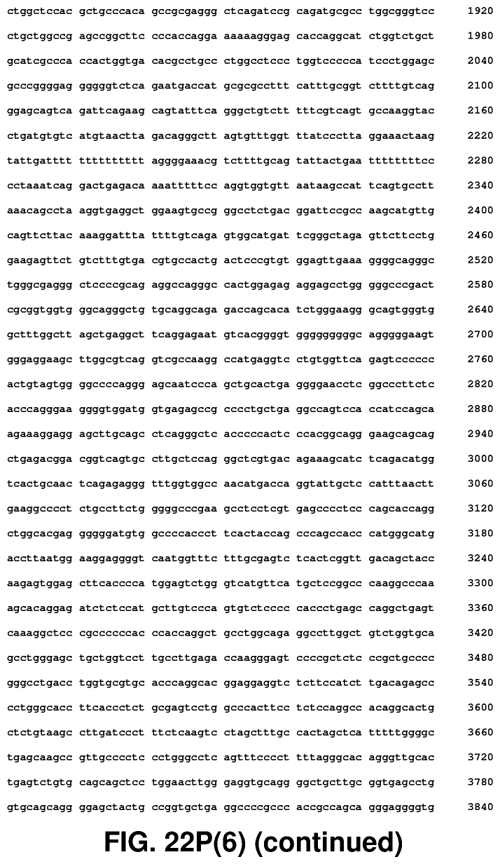

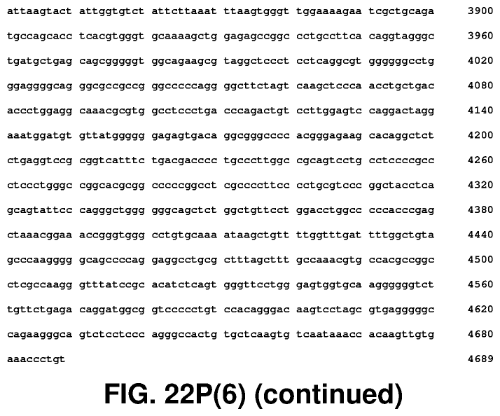

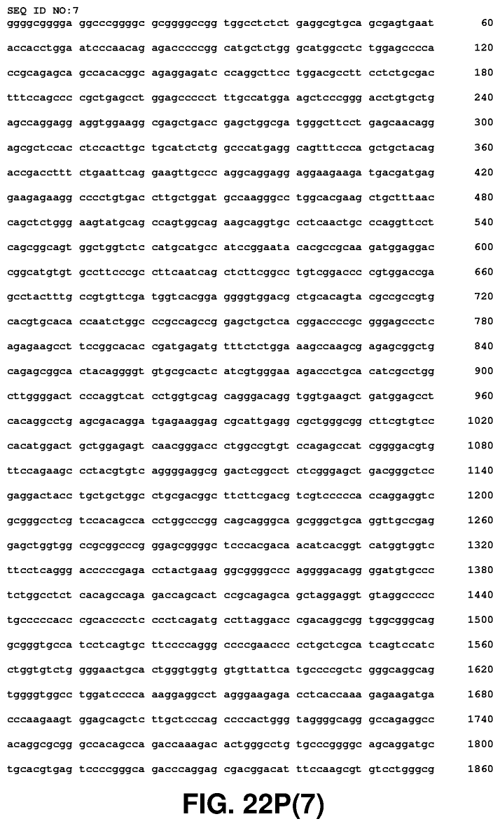

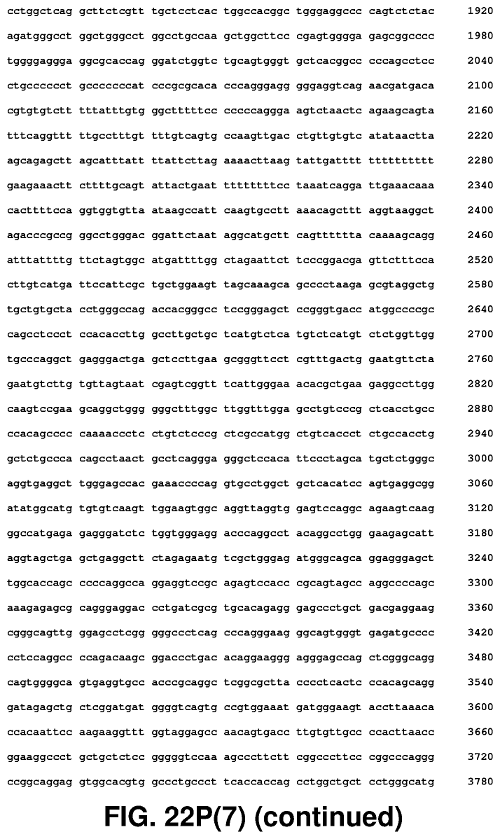

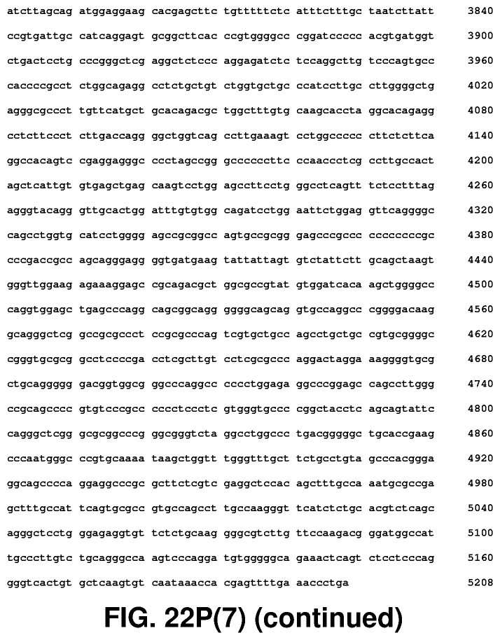

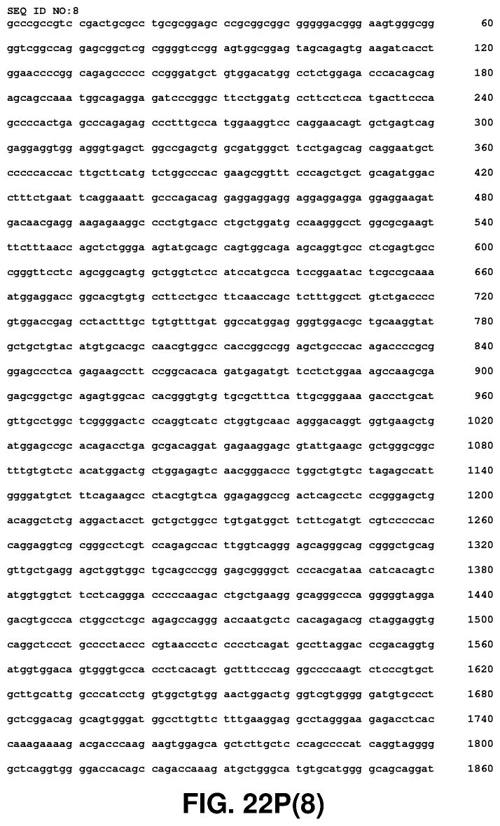

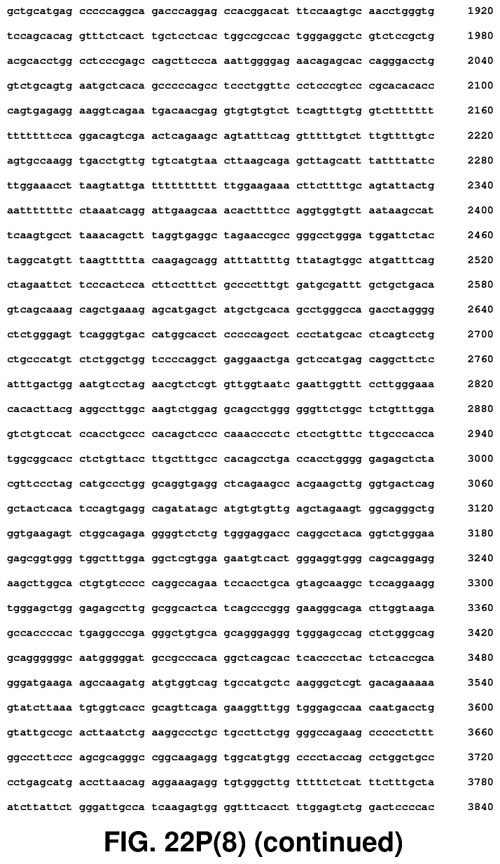

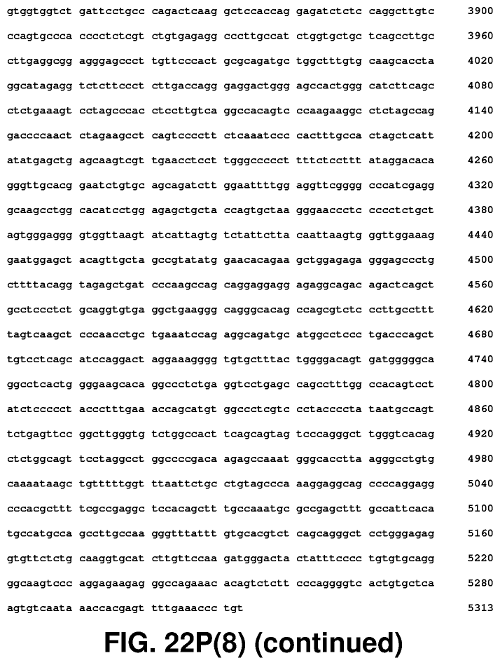

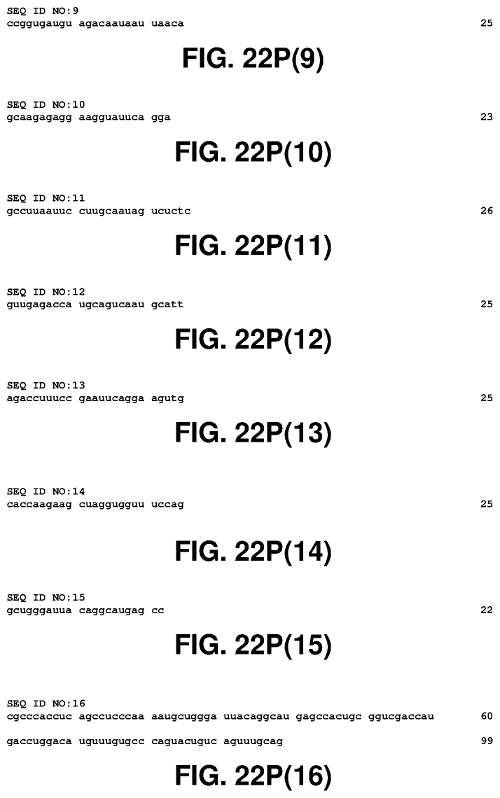

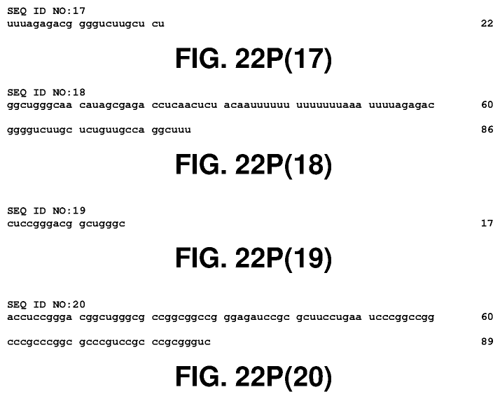

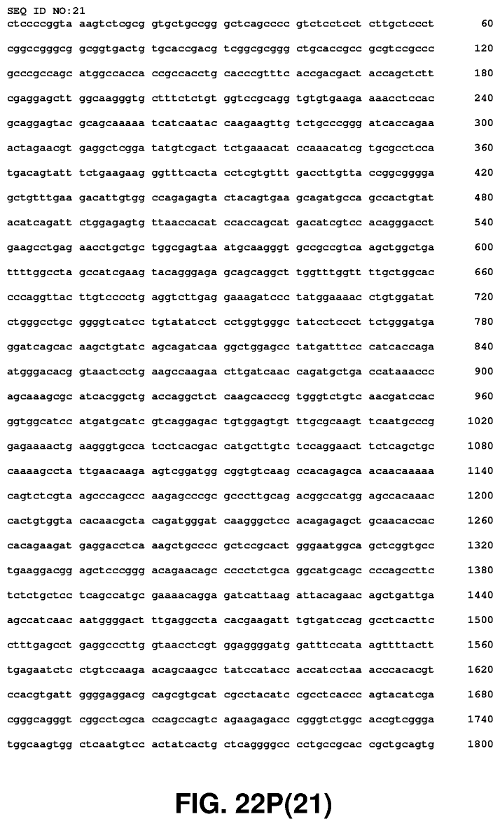

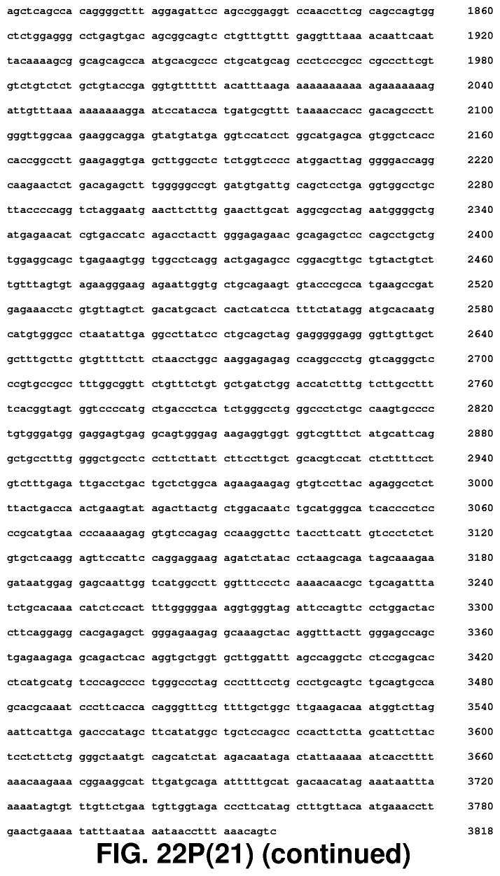

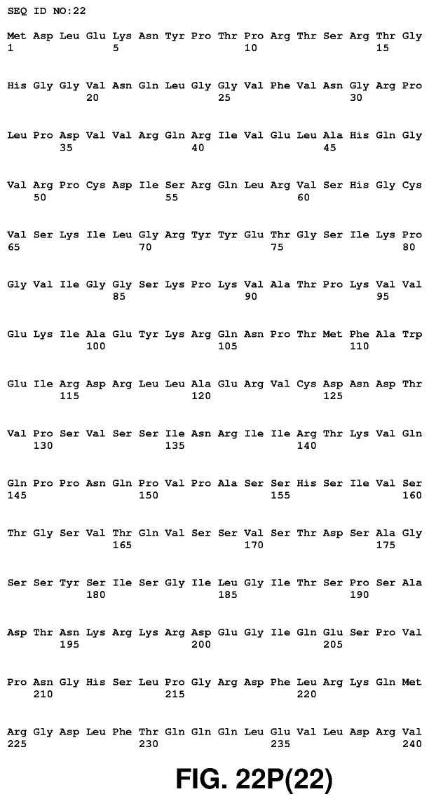

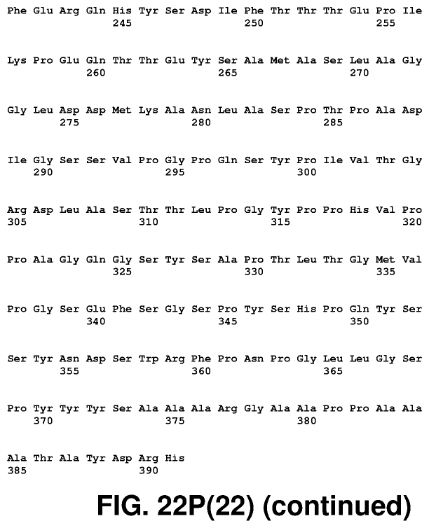

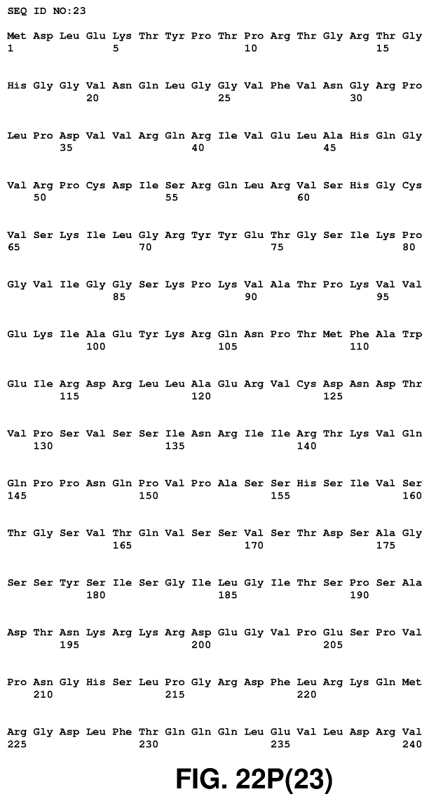

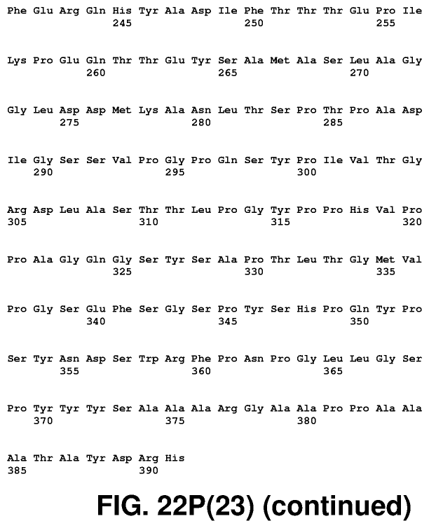

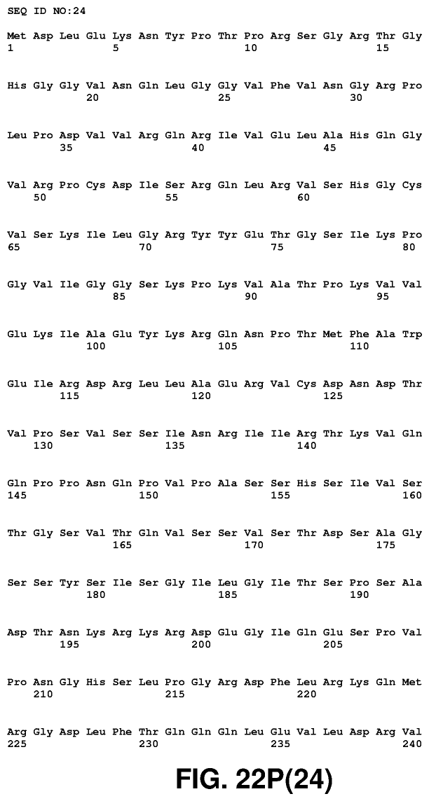

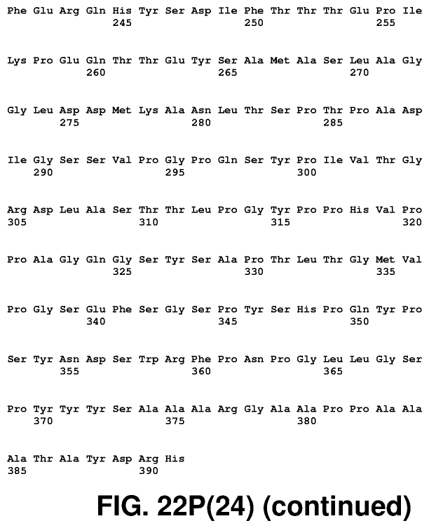

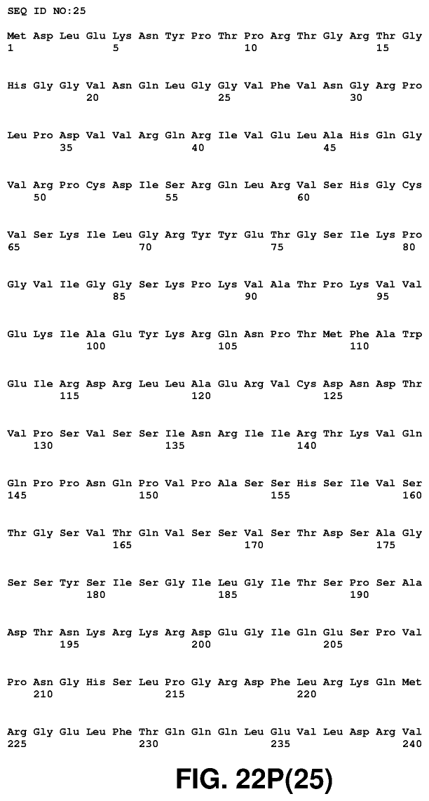

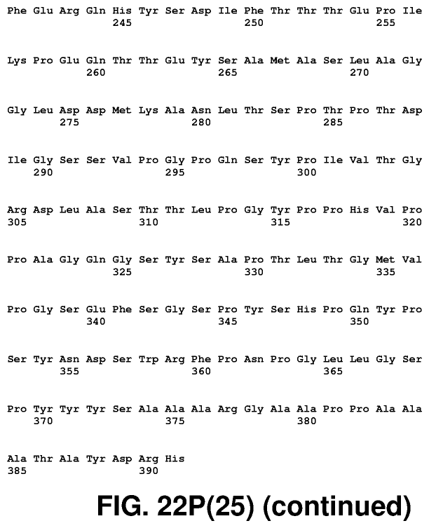

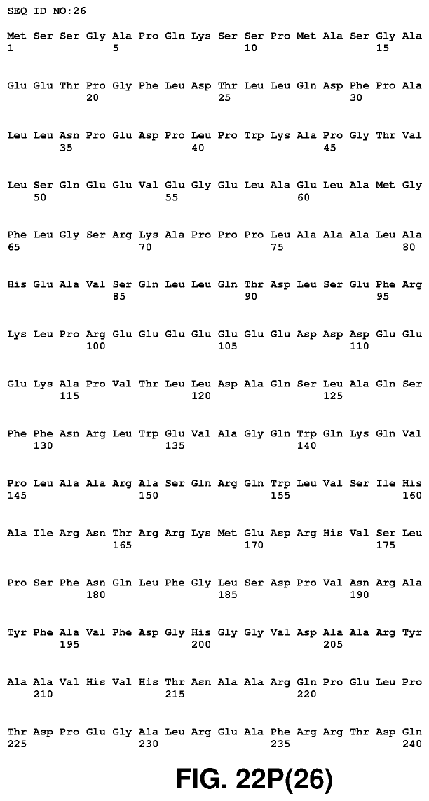

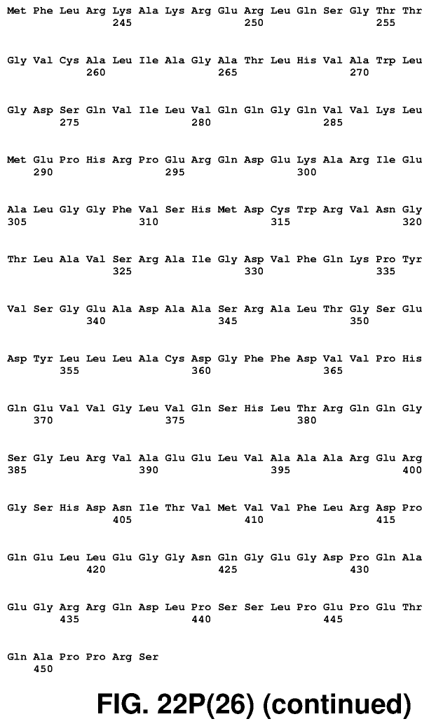

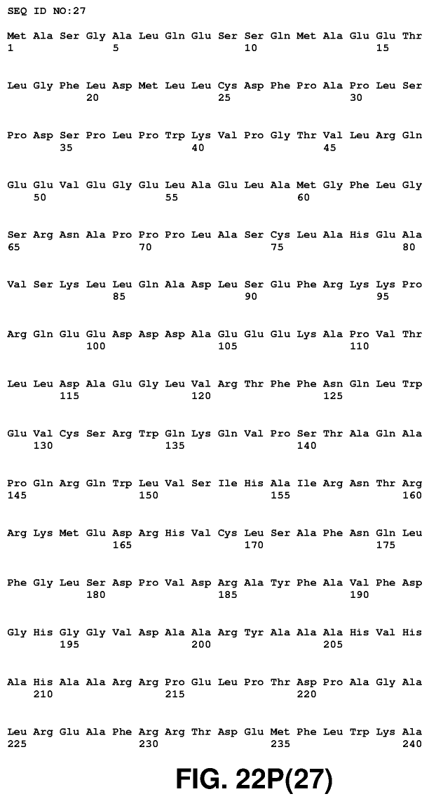

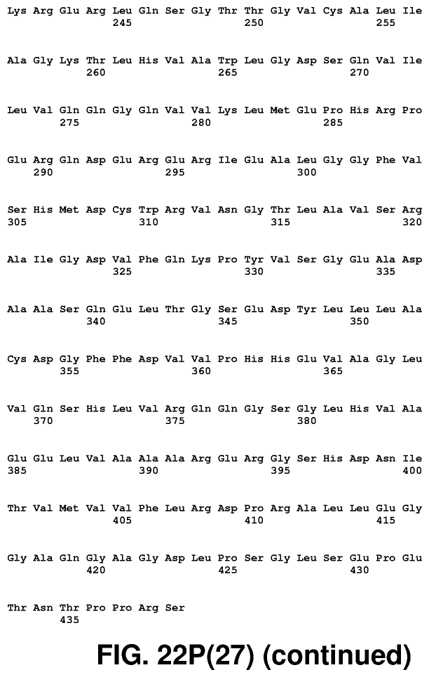

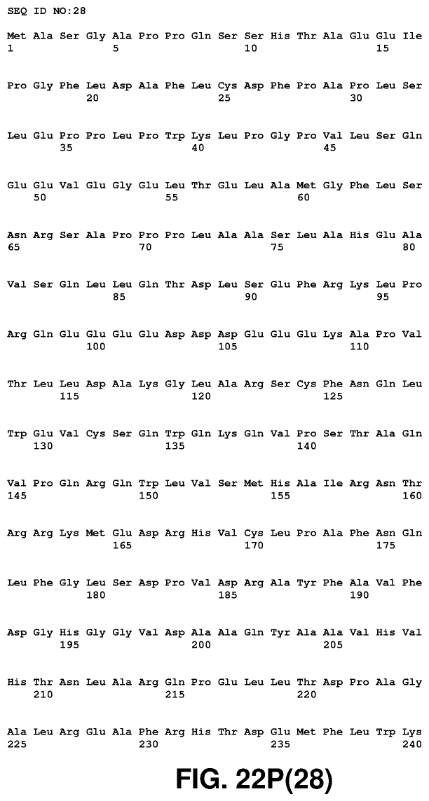

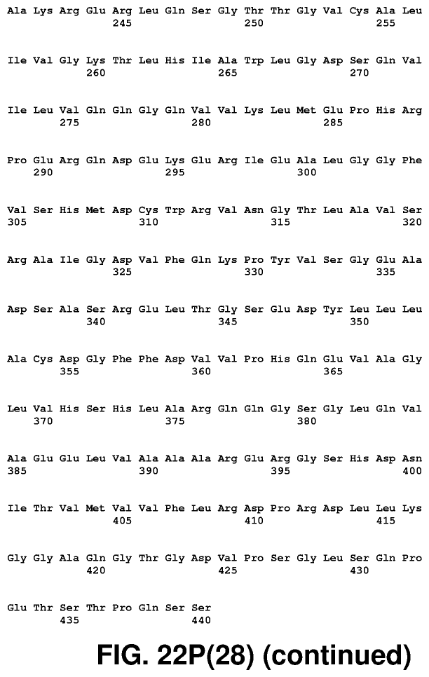

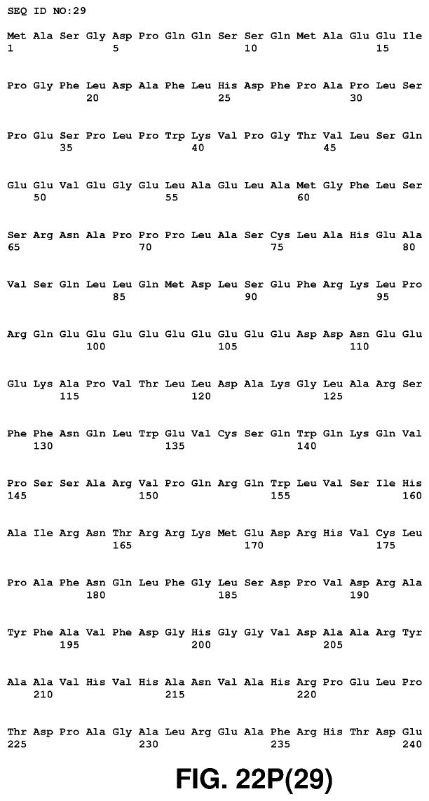

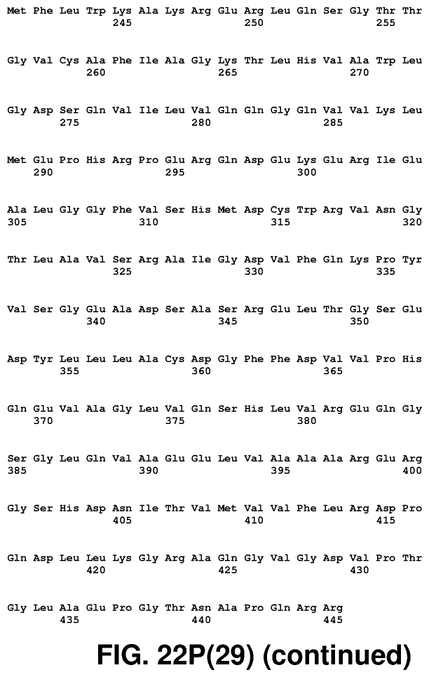

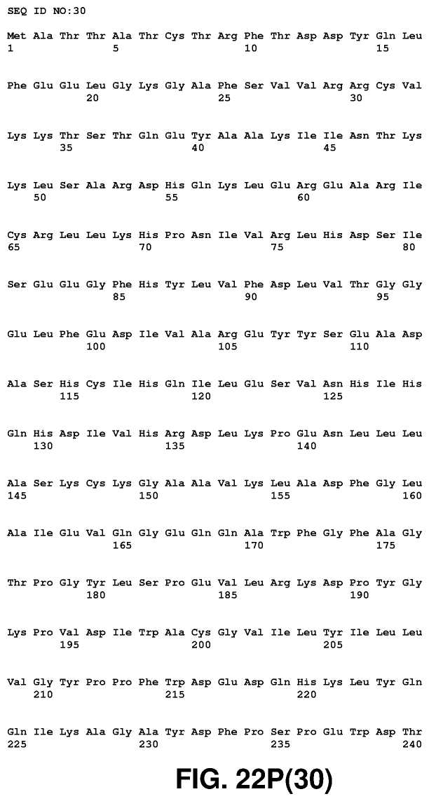

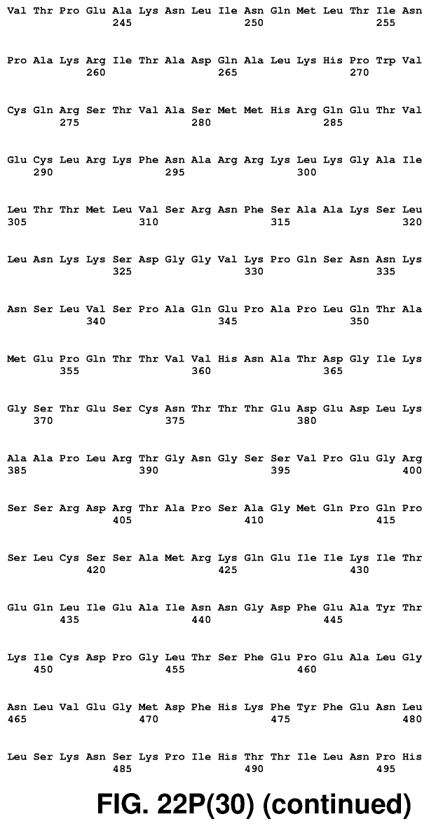

Some embodiments, as shown in FIG. 22N, pertain to a method of preparing at least one cell. In some embodiments, the method comprises selecting a donor 130. In some embodiments, the method comprises providing at least one donor cell from the donor 131. In some embodiments, the method comprises selecting a subject 132. In some embodiments, the method comprises providing at least one subject cell from the subject 133. In some embodiments, the method comprises selecting a patient 136. In some embodiments, the method comprises providing at least one patient cell from the patient 137. In some embodiments, the method comprises exposing the subject cell to the donor cell 134 to provide at least one intermediate cell 135. In some embodiments, the intermediate cell is derived from the subject cell. In some embodiments, the intermediate cell is derived from the donor cell. In some embodiments, the method comprises exposing the patient cell to the intermediate cell to provide a target cell 139. In some embodiments, the target cell is administered to the patient to treat the patient 140. In some embodiments, the donor is the subject (e.g., at an earlier age and/or in a state of improved health). In some embodiments, the donor is the patient (e.g., at an earlier age and/or in a state of improved health). In some embodiments, the subject is the patient (e.g., at an earlier age and/or in a state of improved health).

As shown in FIG. 22O, multiple strategies can be combined to provide treatments for patients. In some embodiments, as shown, the method comprises selecting a donor 130. In some embodiments, the method comprises providing at least one donor cell from the donor 131. In some embodiments, the method comprises selecting a subject 132. In some embodiments, the method comprises providing at least one subject cell from the subject 133. In some embodiments, the method comprises selecting a patient 136. In some embodiments, the method comprises providing at least one patient cell from the patient 137. In some embodiments, the method comprises exposing the subject cell to the donor cell 134 to provide at least one intermediate cell 135. In some embodiments, the intermediate cell is derived from the donor cell. In some embodiments, the method comprises exposing the patient cell to the intermediate cell to provide a target cell 139. In some embodiments, as shown, a cell is acquired and treated by exposing it to one or more different PAX5 gene RNAi(s) and/or PPM1G gene RNAi(s) 141. In some embodiments, as shown, this results in a target cell 142. In some embodiments, as shown in FIG. 22O, the cell can be reintroduced to the patient (e.g., where it was initially isolated from the patient). In some embodiments, one or more small molecule PAX5 and/or PP1F inhibitors are also administered to the patient 143. In some embodiments, the target cell 139 is administered to the patient to treat the patient 140. In some embodiments, as shown, the patient is thereby treated 122. In some embodiments, a method of reducing expression of a paired box 5 (PAX5) gene and reducing expression of a protein phosphatase 1F enzyme (PPM1F) gene in a cell is provided. In some embodiments, the method comprises contacting the cell with one or more interfering RNA(s) (RNAi(s)) comprising one or more of SEQ ID NOs:9-20 maintaining the cell for a time sufficient to obtain inhibition of the PAX5 gene and the PPM1F gene, thereby reducing expression of the PAX5 gene and the PPM1F gene in that cell to provide a target cell. In some embodiments, the RNAi(s) act on the patient cell and/or the subject cell to provide the target cell and/or the intermediate cell. In some embodiments, the method comprises contacting the cell with one or more polycyclic aromatic compounds that antagonize or reduce the expression of PAX5 and/or PPM1F. In some embodiments, one or more of the donor, the subject, and/or the cells therefrom can be treated with the RNAi(s) and/or small molecule PAX5/PP1F inhibitors prior to the exposure of the patient cell to the intermediate cell. For instance, in some embodiments, any one of the donor cell, the subject cell, the intermediate cell, or the target cell, can be treated with the RNAi(s). In some embodiments, any one of the donor, the subject, or the patient can be administered small molecule PAX5/PP1F inhibitors prior to cell harvesting.

Some embodiments pertain to a method of treatment comprising administering to a subject in need thereof a therapeutically effective amount of the pharmaceutical composition. In some embodiments, the subject has one or more medical conditions or age-related disorders selected from the group consisting of arthritis, atherosclerosis, breast cancer, cardiovascular disease, cataracts, chronic obstructive pulmonary disease, colorectal cancer, hypertension, osteoporosis, periodontitis, type 2 diabetes, and Alzheimer's disease.

### Additional Protocols for Anti-Aging Therapy

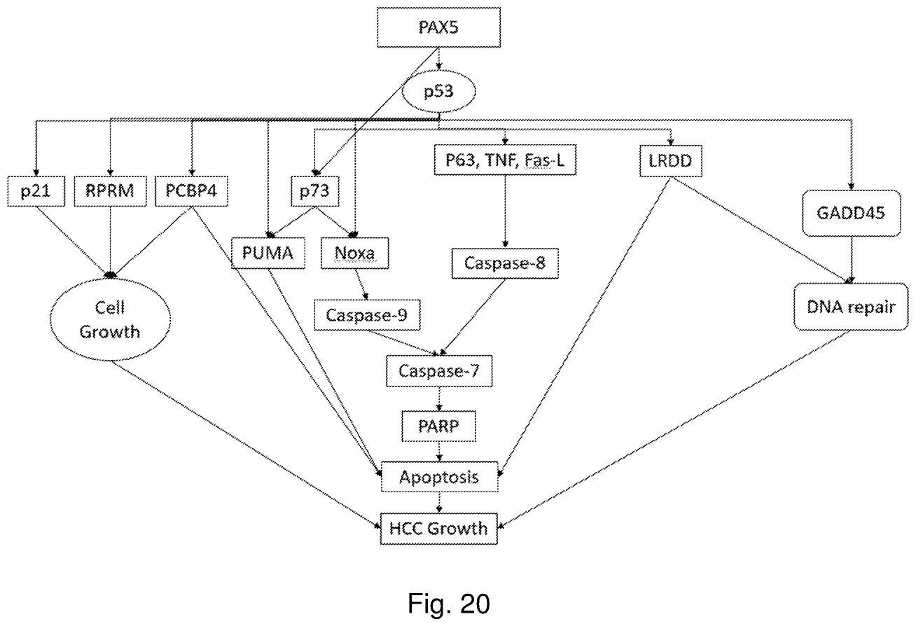

As described herein, a role for systemic factors in the young circulation of heterochronic donors induces rejuvenation of cognitive, cardiac and skeletal muscle function of matched aged animals. In some embodiments, the blood of, for example, young mice compartmentalizes pleiotropic factors that prevent age-associated tissue dysfunction. In some embodiments, a model that allows the measurement of the effects of factors released from young blood cells on the function of like aged cells was developed (as shown in FIG. 20).

In some embodiments, mobilized peripheral blood, which comprises a heterogeneous population of cells, such as hematopoietic stem cells (HSCs), hematopoietic progenitors, mesenchymal stem cells (MSCs), endothelial progenitor cells (EPCs) and mature immune cells, collected from healthy young donors and aged patients is incubated in a transwell culture that allows for the exchange of soluble factors but no direct interaction. In some embodiments, prolonged exposure of the aged cells to young, secreted factors boosts cytotoxic immune function and hematopoietic differentiation potential to levels similar to that observed in young cells. In some embodiments, the process is termed “cellular restoration.” In some embodiments, this process is in part based on a role for exosome-mediated intercellular communication.

In some embodiments, restorative effects were not due to cellular transformation. In some embodiments, transplanting restored aged cells into immunodeficient mice provided no safety concerns over a 1 year period. In some embodiments, these findings suggest that soluble factors isolated from mobilized blood have potential therapeutic value for a number of indications related to age-associated immunosenescence as well as a preventative approach to promote healthy aging. In some embodiments, these preclinical findings were translated into a healthy anti-aging treatment for aged individuals. In some embodiments, the function to the aging or defective hematopoietic and immune systems can be restored and/or improved, thereby promoting the body's endogenous defense and surveillance mechanisms to combat diseases of aging such as cancer and infectious disease.

In some embodiments, the process includes targeting declining function of aging hematopoietic stem cells (HSCs) as a means to treat and delay the onset of age-related diseases. Aging is a highly complex biological process and the leading risk factor for the chronic diseases that account for the bulk of morbidity, mortality, and health care costs. In some embodiments, restoration of aged tissue by youthful factors may decrease age-related disease.

In some embodiments, “restoration” of aged tissue and cells is possible. In some embodiments, blood from young animals can rejuvenate old animals, and cell-free factors isolated from the stem cells of young animals can rejuvenate the stem cells of older animals. In some embodiments, factors (e.g., from exosomes including miRNA as disclosed elsewhere herein) produced from blood cells of younger donors restore function of blood cells from aged donors.

In some embodiments, the studies disclosed herein have proven efficacious, and have demonstrated no significant safety concerns in various studies with post-treatment monitoring up to 1 year or longer. In some embodiments, a clinical protocol has been developed translating these findings into a healthy aging treatment for aged individuals. In some embodiments, restoration of the function to the aging hematopoietic and immune systems is accomplished. In some embodiments, this immunotherapy promotes restoration of the aging body's endogenous immune defense and surveillance mechanisms, thereby preventing age-related diseases such as cancer and infectious disease.

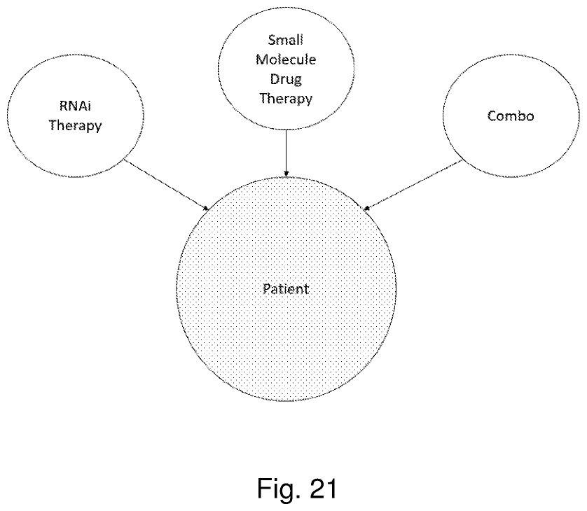

In some embodiments, healthy aged (e.g., >60 y/o) and young (e.g., 18-29 y/o) individuals were recruited for stem cell mobilization and collection. In some embodiments, healthy donors are dosed with the mobilizing agent (e.g., G-CSF (Neupogen®)) for a period of time (e.g., 5 days), with blood cell collection thereafter (e.g., on the 6th day) by leukapheresis. In some embodiments, the aged stem cell source can be CD34+ (as shown in FIG. 21) and mesenchymal stem cells derived from adipose tissue (stromal vascular fraction). In some embodiments, young and aged cells can be cultivated in the disclosed transwell co-culture system so that factors released by the young cells are able to interact with the aged cells through a semi-permeable membrane filter that prevents direct cell-cell interaction.

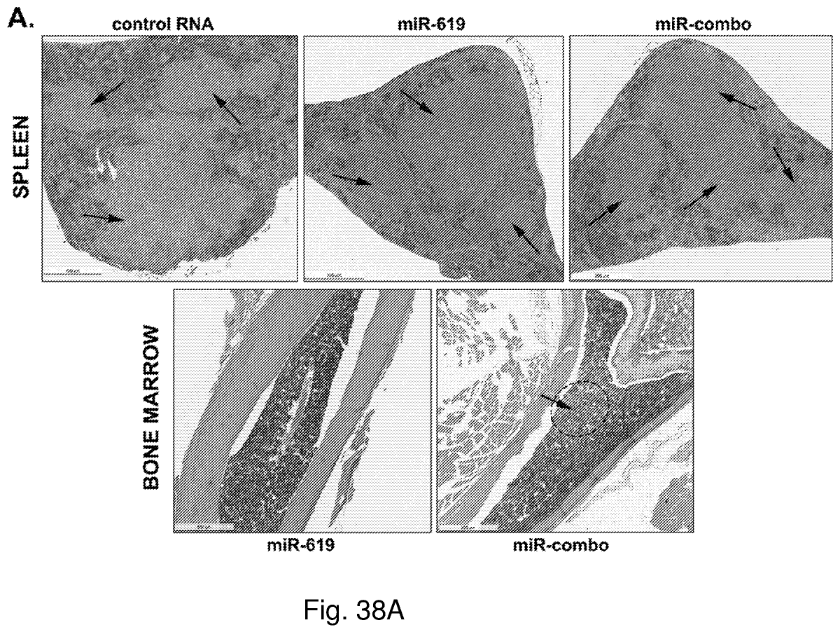

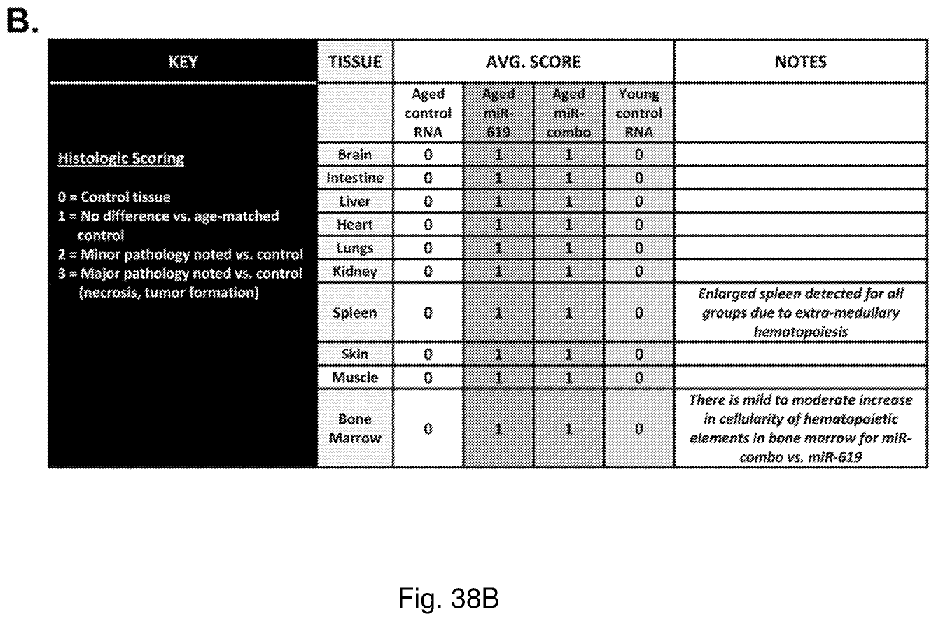

In some embodiments, after a period of culture (e.g., 7 days of culture), the functionally restored aged cells are washed and formulated in a saline solution for intravenous infusion into, for example, the same aged individual whose blood cells were originally collected. In some embodiments, after infusion, the patient will be monitored at 2, 6, 12 and 24 months posttreatment (or as shown in, for example FIG. 38).

In some embodiments, to measure clinical efficacy, a specific panel of assays (see an embodiment below, Assessment of Clinical Safety & Efficacy) can be conducted pre-treatment (baseline) as well as at each monitoring time point by peripheral blood collection. In some embodiments, these will include a full CBC assessment, immune cell phenotyping to determine key immune aging metrics such as myeloid to lymphoid ratio and CD4/CD8 ratio, as well as measurement of immune response to mitogen and antigen stimulation. In some embodiments, results from these measures will be compared to baseline, with evaluation of the need for follow-up treatment at 12 months based on loss of efficacy. In some embodiments, the restorative effects are sustainable for equal to or at least about: 6 months, 12 months, 2 years, or ranges including and/or spanning the aforementioned values. In some embodiments, individuals may be given follow-up treatment given the aging process is on-going.

In some embodiments, a stromal vascular fraction (SVF) is used in the treatment methods disclosed herein. In some embodiments, the SVF contains a mixture of endothelial cells, endothelial progenitor cells (EPC or CD34+ cells), and mesenchymal stem cells (MSC). In some embodiments, this SVF is derived from lipoaspirate using mechanical (lipogems) or enzymatic (collagenase) based digestion followed by elution of the cells resulting in elimination of the enzyme.

In some embodiments, the techniques and methods disclosed herein improve one or more of the biological aging hallmarks that are linked to aging including one or more of: decreasing genomic instability (lowering gene mutation rate), decreasing telomere attrition (e.g., lowering replicative stress), decreasing the loss of proteostasis (e.g., decreasing the incidence of protein misfolding, aggregation, and/or degradation), decreasing the deregulation of nutrient sensing (e.g., decreasing altered cellular energy metabolism), decreasing mitochondrial dysfunction (e.g., decreasing oxidative stress), decreasing epigenetic alteration (e.g., decreasing cellular senescence, ongogenesis, etc.), decreasing cellular senescence (e.g., decreasing incidence of an altered tissue microenvironment), decreasing stem cell exhaustion (e.g., decreasing the disruption of cellular homeostasis and tissue repair), and decreasing senescence related altered intercellular communications (which causes local and systemic tissue disruption).

In some embodiments, the causal elements of aging occur at the macromolecular and/or organelle level and can be altered using the disclosed methods. Over time, this continuous intracellular stress synergistically leads to impairment of cellular function. Consequently, cellular homeostasis and the tissue microenvironment are disrupted due to stem cell exhaustion, altered intercellular communication and increased cellular senescence. These changes ultimately lead to a decline in tissue function and the manifestation of disease. In some embodiments, by targeting aging at the cellular level one or more of these dysfunctions can be treated or prevented. In some embodiments, the methods disclosed herein prevent age-related disease, since cellular dysfunction directly precedes tissue disability. In some embodiments, the methods disclosed herein treat the disruption of the tissue microenvironment which is a hallmark of organismal aging. In some embodiments, microenvironmental disruption has dramatic consequences to the cellular niche, often leading to stem cell exhaustion and improper tissue homeostasis. In some embodiments, stem cell exhaustion results from an inability of stem cells to continually replenish a tissue with differentiated cells that are necessary to maintain tissue function. In some embodiments, to properly sustain stem cell pools, a delicate balance of self-renewal, proliferation and quiescence is required and is achieved using one or more methods disclosed herein. In some embodiments, when operating properly, the microenvironment houses a variety of cell types acting in concert to maintain tissue homeostasis and promote tissue function. In some embodiments, a component of coordinating such activity is the proper exchange of information from cell to cell, or intercellular communication, which can be achieved using on or more methods disclosed herein. In some embodiments, intercellular communication can be cell contact dependent or independent.

Contact independent communication, termed paracrine communication, is mediated in part by the release of microvesicles, such as exosomes. Exosomes are small membrane vesicles derived from multivesicular bodies that are released by all cell types. These vesicles contain a subset of proteins, lipids and nucleic acids from the parent cell. In some embodiments, exosomes have important roles in intercellular communication, both locally and systemically, as they shuttle their contents, including proteins, lipids and nucleic acids, between cells. In some embodiments, miRNAs taken up by recipient cells can change target cell behavior by classical miRNA-induced silencing of target mRNAs. In some embodiments, this form of intercellular communication is involved in numerous physiological processes, including immune regulation. In some embodiments, as disclosed elsewhere herein, exosomes, RNA, and/or small molecules can be used in one or more of the methods disclosed herein to provide target cells. In some embodiments, once formulated the target cells can be transported and/or stored (e.g., cryogenically) as disclosed herein.

In some embodiments, within the cellular aging field, exosomes from young adult stem cells can increase the lifespan of patients. In some embodiments, exosomes mediate these therapeutic effects and have a role in intercellular shuttle of miRNA in regulating a number of aging-related signaling pathways in targeted cells.

In some embodiments, youthful factors (GDF-11 and factors as disclosed elsewhere herein) found in blood plasma can restore tissue function, infusion of plasma from young animals into aged. In some embodiments, transplantation of mesenchymal stem cells (MSCs) from young donors into aged patients can be used to delay aging, decrease cell dysfunction, and extend lifespan (e.g., by equal to or greater than 25%, 17% 15%, 10%, 5%, or ranges including and/or spanning the aforementioned values). In some embodiments, the methods disclosed herein involve the hematopoietic and immune systems are vital components of how an organism functions. In some embodiments, blood cells (e.g., cells from blood, including specific isolated cells, including those isolated based on their expression of specific cell markers etc.) are used and perfuse most tissues of the body and serve local housekeeping and surveillance roles within the tissue microenvironment.

As certain systems age, their diminishing functions lead to compensatory increases in immune-related diseases, including cancer. In some embodiments, the hematopoietic and immune systems at least partially depend on adult hematopoietic stem cells (HSCs) function throughout an organism's lifetime to generate progenitor cells and mature effector blood cells. Loss of HSC function through “immuno-senescence” is a major source of morbidity and mortality, as decreased immune surveillance leads to increased incidences of cancer, infectious disease and immune-related disorders. In some embodiments, these issues or others are treated with the methods, compositions, and protocols disclosed herein. In some embodiments, the declining function of aging HSC is used as a means to treat and delay the onset of age-related diseases. In some embodiments, a heterochronic cell culture (transwell with cells from differently aged patients) is used to stimulate production of rejuvenating factors from young blood cells, which, when applied to aged HSCs, restore youthful function. In some embodiments, the methods disclosed herein modulate elements of aging-related pathways to promote recovery of aged HSC function. In some embodiments, replication stress as a driver of HSC aging and DNA helicase to facilitate HSC rejuvenation, is targeted. In some embodiments, altered cellular energy metabolism to reverse aging of the immune system is targeted.

In some embodiments, hematopoietic and immune systems, as targeted and rejuvenated by one or more of the methods disclosed herein, are vital components of how an organism functions. In some embodiments, blood cells perfuse most tissues of the body and serve local housekeeping and surveillance roles within the tissue microenvironment. As these systems age, their diminishing functions lead to compensatory increases in immune-related diseases, including cancer. The hematopoietic and immune systems depend in part on adult hematopoietic stem cell (HSC) function throughout an organism's lifetime to generate progenitor cells and mature effector blood cells. Loss of HSC function through immunosenescence is a major source of morbidity and mortality, as decreased immune surveillance leads to increased incidences of cancer, infectious disease and immune-related disorders. In some embodiments, the declining function of aging HSCs is used herein as a means to treat and delay the onset of age-related disease and the other dysfunctions disclosed herein. In some embodiments, a heterochronic cell culture is used to stimulate production of rejuvenating factors from young blood cells that restore youthful function of aged HSCs. In some embodiments, restored HSCs, or purified youthful factors alone, target the aged cells (e.g., in the bone marrow niche) to promote tissue homeostasis and to limit dysfunction. In some embodiments, the restored hematopoietic system results in a competent immune system. In some embodiments, life can be prolonged and/or the treatment increases quality of life. In some embodiments, the hematopoietic cells as well as the microenvironment are restored to a functionally younger organ. In some embodiments, a competent immune system is provided and impacts other organs, including the brain.

In some embodiments, the methods restore function to the aging hematopoietic and immune systems to promote restoration of the aging body's endogenous immune defense and surveillance capabilities. In some embodiments, the methods disclosed herein successfully and safely translate an autologous, adoptive cell therapy protocol that harnesses the restorative ability of young blood cells to improve aging immune and stem cell function to clinical populations. In some embodiments, the methods disclosed herein demonstrate that the approach is safe (e.g., at 2 months, 6 months, 1 year, 2 years, or ranges including and/or spanning the aforementioned values) after treatment by observing no treatment related serious adverse events. In some embodiments, the methods disclosed herein demonstrate improvement in biological and clinical efficacy measures (e.g., at 2 months, 6 months, 1 year, 2 years, or ranges including and/or spanning the aforementioned values) after treatment

### MicroRNAs and Small Molecules for Use in Some Embodiments

Also disclosed herein are methods of preparing target cells using one or more of the following: microRNAs, small molecules, and combinations thereof.

The term “ribonucleotide” and the phrase “ribonucleic acid” (RNA), as used herein, refer to a modified or unmodified nucleotide or polynucleotide comprising at least one ribonucleotide unit. A ribonucleotide unit comprises an oxygen attached to the 2′ position of a ribosyl moiety having a nitrogenous base attached in N-glycosidic linkage at the 1′ position of a ribosyl moiety, and a moiety that either allows for linkage to another nucleotide or precludes linkage. RNA are a class of single-stranded molecules, which in their natural state, can be transcribed from DNA in the cell nucleus or in the mitochondrion or chloroplast, containing along the strand a linear sequence of nucleotide bases that is complementary to the DNA strand from which it is transcribed: the composition of the RNA molecule is identical with that of DNA except for the substitution of the sugar ribose for deoxyribose and the substitution of the nucleotide base uracil for thymine. RNA may be in the form of siRNA, asymmetrical interfering RNA (aiRNA), microRNA (miRNA), mRNA, tRNA, rRNA, RNA, viral RNA (vRNA), and combinations thereof.

The term “deoxynucleotide”, as used herein, refers to a nucleotide or polynucleotide lacking an OH group at the 2′ or 3′ position of a sugar moiety with appropriate bonding and/or 2′, 3′ terminal dideoxy, instead having a hydrogen bonded to the 2′ and/or 3′ carbon.

The terms “deoxyribonucleotide” and “DNA”, as used herein, refer to a nucleotide or polynucleotide comprising at least one ribosyl moiety that has an H at its 2′ position of a ribosyl moiety instead of an OH. DNA may be in the form of, e.g., antisense molecules, plasmid DNA, pre-condensed DNA, a PCR product, vectors (PAC, BAC, YAC, artificial chromosomes), expression cassettes, chimeric sequences, chromosomal DNA, or derivatives and combinations of these groups.

“Polynucleotide,” or “nucleic acid,” as used interchangeably herein, refers to polymers of nucleotides of any length, and includes DNA and RNA. The nucleotides can be deoxyribonucleotides, ribonucleotides, unmodified nucleotides or bases, modified nucleotides or bases, and/or their analogs, or any substrate that can be incorporated into a polymer by DNA or RNA polymerase. A polynucleotide may comprise modified nucleotides, such as methylated nucleotides and their analogs. The term “nucleic acid” as used herein refers to a polymer containing at least two deoxyribonucleotides or ribonucleotides in either single- or double-stranded form and includes DNA and RNA. Nucleic acids include nucleic acids containing known nucleotide analogs or modified backbone residues or linkages, including for example locked nucleic acid (LNA), unlocked nucleic acid (UNA), and zip nucleic acid (ZNA), which can be synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylases, and alkylhalides. “Oligonucleotide,” as used herein, generally refers to short, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides. In some embodiments, the polynucleotides disclosed herein (e.g., RNAi(s)) can include one or more nucleotides that is not naturally occurring to, for example, improve the stability of the polynucleotide. Some non-natural nucleotide modifications can include: phosphorothioate linkages, boranophosphate linkages, locked nucleic acids, 2′-modified RNA, 4′-thio modified RNA, ribo-difluorotoluyl nucleotides, uncharged nucleic acid mimics, siRNA conjugates including but not limited to peptide additions or polyethylene glycol. In some embodiments, the polynucleotide does not include non-natural nucleotides.

As used herein, “sense sequence” refers to a polynucleotide or region that has the same nucleotide sequence, in whole or in part, as a target nucleic acid such as a messenger RNA or a sequence of DNA. When a sequence is provided, by convention, unless otherwise indicated, it is the sense sequence (or region), and the presence of the complementary antisense sequence (or region) is implicit.

As used herein, “antisense sequence” refers to a polynucleotide or region of a polynucleotide that is substantially complementary (e.g., 80% or more) 90, 85, 98, 99, or 100% complementary to a target nucleic acid of interest. An antisense sequence can be composed of a polynucleotide region that is RNA, DNA or chimeric RNA/DNA. Any nucleotide within an antisense sequence can be modified by including substituents coupled thereto, such as in a 2′ modification. The antisense sequence can also be modified with a diverse group of small molecules and/or conjugates. For example, an antisense sequence may be complementary, in whole or in part, to a molecule of messenger RNA, an RNA sequence that is not mRNA (e.g., tRNA, rRNA, hnRNA, negative and positive stranded viral RNA and its complementary RNA) or a sequence of DNA that is either coding or non-coding.

As used herein, the term “complementary” refers to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G), or in any other manner that allows for the formation of stable duplexes.

As used herein, “perfect complementarity” or “100% complementarity” refers to the situation in which each nucleotide unit of one polynucleotide strand or region can hydrogen bond with each nucleotide unit of a second polynucleotide strand or region. Less than perfect complementarity refers to the situation in which some, but not all, nucleotide units of two strands or two regions can hydrogen bond with each other. For example, for two 19-mers, if 17 base pairs on each strand or each region can hydrogen bond with each other, the polynucleotide strands exhibit 89.5% complementarity.

As used herein, “mismatch” includes situations in which Watson-Crick base pairing does not take place between a nucleotide of a antisense sequence and a nucleotide of a sense sequence, where the nucleotides are flanked by a duplex comprising base pairs in the 5′ direction of the mismatch beginning directly after (in the 5′ direction) the mismatched position and in the 3′ direction of the mismatch beginning directly after (in the 3′ direction) the mismatched position. Examples of mismatches include, without limitation, an A across from a G, a C across from an A, a U across from a C, an A across from an A, a G across from a G, a C across from a C, and so on. Mismatches also include an abasic residue across from a nucleotide or modified nucleotide, an acyclic residue across from a nucleotide or modified nucleotide, a gap, or an unpaired loop. In its broadest sense, a mismatch includes any alteration at a given position that decreases the thermodynamic stability at or in the vicinity of the position where the alteration appears, such that the thermodynamic stability of the duplex at the particular position is less than the thermodynamic stability of a Watson-Crick base pair at that position. Mismatches include a G across from an A, and an A across from a C. Some embodiments of a mismatch comprise an A across from an A, G across from a G, C across from a C, and U across from a U.

As used herein, “silencing” refers to an RNAi-mediated reduction in gene expression that can be measured by any number of methods including reporter methods such as for example luciferase reporter assay, PCR-based methods, Northern blot analysis, Branched DNA, western blot analysis, and other techniques.

The term “interfering RNA” or “RNAi” or “interfering RNA sequence” refers to single-stranded or double-stranded RNA, including short interfering RNA (siRNA), microRNA (miRNA), circular RNAs (circRNAs), short hairpin RNAs (shRNAs), long non-coding RNAs (lncRNAs); piwi-interacting RNAs (piRNA), small nucleolar RNA (snoRNAs), tRNA-derived small RNA (tsRNA), small rDNA-derived RNA (srRNA), or a small nuclear RNA (U-RNA). The sequence will not be the full length of the target gene, but some fragment thereof. These sequences can be used for reducing or inhibiting the expression of a target gene or sequence (e.g., by mediating the degradation or inhibiting the translation of mRNAs which are complementary to the interfering RNA sequence) when the interfering RNA is in the same cell as the target gene or sequence. In some embodiments, interfering RNA thus refers to the single-stranded RNA that is complementary to a target mRNA sequence or to the double-stranded RNA formed by two complementary strands or by a single, self-complementary strand. Interfering RNA may have substantial or complete identity to the target gene or sequence, or may comprise a region of mismatch (i.e., a mismatch motif). The sequence of the interfering RNA can correspond to the full-length target gene, or a subsequence thereof. Interfering RNA includes “small-interfering RNA” or “siRNA,” e.g., interfering RNA of about 15-60, 15-50, or 5-40 (duplex) nucleotides in length, more typically about 15-30, 15-25, or 19-25 (duplex) nucleotides in length, and is, in some embodiments, about 20-30, 20-24, 21-22, or 21-23 (duplex) nucleotides in length (e.g., each complementary sequence of the double-stranded siRNA is 15-60, 15-50, 15-40, 15-30, 15-25, 19-25, or 20-30 nucleotides in length, about 20-24, 21-22, or 21-23 nucleotides in length, and the double-stranded siRNA is about 15-60, 15-50, 15-40, 5-30, 5-25, or 19-25 base pairs in length, preferably about 8-22, 9-20, or 19-21 base pairs in length). siRNA duplexes may comprise 3′ overhangs of about 1 to about 4 nucleotides or about 2 to about 3 nucleotides and 5′ phosphate termini. Examples of siRNA include, without limitation, a double-stranded polynucleotide molecule assembled from two separate stranded molecules, wherein one strand is the sense strand and the other is the complementary antisense strand; a double-stranded polynucleotide molecule assembled from a single stranded molecule, where the sense and antisense regions are linked by a nucleic acid-based or non-nucleic acid-based linker; a double-stranded polynucleotide molecule with a hairpin secondary structure having self-complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA molecule. Preferably, siRNA are chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA. Preferably, dsRNA are at least 50 nucleotides to about 100, 200, 300, 400, or 500 nucleotides in length A dsRNA may be as long as 1000, 1500, 2000, 5000 nucleotides in length, or longer. The dsRNA can encode for an entire gene transcript or a partial gene transcript. In certain instances, siRNA may be encoded by a plasmid (e.g., transcribed as sequences that automatically fold into duplexes with hairpin loops). A small hairpin RNA or short hairpin RNA (shRNA) is a sequence of RNA that makes a tight hairpin turn that can be used to silence gene expression via RNA interference. The shRNA hairpin structure is cleaved by the cellular machinery into siRNA, which is then bound to the RNA-induced silencing complex (RISC). This complex binds to and cleaves mRNAs which match the siRNA that is bound to it. Suitable length of the interference RNA are about 5 to about 200 nucleotides, or 10-50 nucleotides or base pairs or 15-30 nucleotides or base pairs. In some embodiments, the interference RNA is substantially or completely complementary (such as at least about: 60%, 70%, 80%, 90%, 95%, 98%, 99%, 99.9%, 99.99%, 100%, or ranges including and/or spanning the aforementioned values) the corresponding target gene. In some embodiments, the interference RNA is modified, for example by incorporating non-naturally occurring nucleotides. In some embodiments, an “interfering RNA” or RNAi is an RNA having a structure characteristic of molecules that function to mediate inhibition or interference with of gene expression through an RNAi mechanism or an RNA strand comprising at least partially complementary portions that hybridize to one another to form such a structure. When an RNA comprises complementary regions that hybridize with each other, the RNA will be said to self-hybridize. An RNAi suitable for use in the present disclosure may include a portion that is substantially complementary to a target gene. An RNAi, optionally includes one or more nucleotide analogs or modifications. One of ordinary skill in the art will recognize that RNAi(s) that are synthesized in vitro can include ribonucleotides, deoxyribonucleotides, nucleotide analogs, modified nucleotides or backbones, etc., whereas RNAi(s) synthesized intracellularly, e.g., encoded by DNA templates, typically consist of RNA, which may be modified following transcription. Of particular interest herein are short RNAi(s), i.e., RNAi(s) consisting of one or more strands that hybridize or self-hybridize optionally having one or more mismatched or unpaired nucleotides within the duplex. RNAi(s) include short interfering RNAs (siRNAs), short hairpin RNAs (shRNAs), and other RNA species that can be processed intracellularly to produce shRNAs including, but not limited to, RNA species identical to a naturally occurring miRNA precursor or a designed precursor of an miRNA-like RNA. In some embodiments, RNAi refers to dsRNA-induced gene silencing, a cellular process that degrades RNA homologous to one strand of the dsRNA. Methods of mediating the RNAi effect involve small interfering RNA (siRNA), short hairpin RNA (shRNA) and bi-functional shRNA. The interfering RNAs (e.g., siRNA, shRNA), when introduced into cells, can be used to silence genes in mammalian systems where long dsRNAs prompt protein kinase R (PKR), RNase L, and interferon activities that result in non-specific RNA degradation and general shutdown of protein synthesis. Herein any RNA that can be used in the present disclosure to reduce the expression of mRNA of a target biomolecule are collectively termed RNAi.

As used herein, “short, interfering RNA” (siRNA) refers to a nucleic acid that may include a double-stranded portion between about 15-29 nucleotides in length and optionally further comprises a single-stranded overhang (e.g., 1-6 nucleotides in length) on either or both strands. The double-stranded portion is typically between 17-21 nucleotides in length, e.g., 19 nucleotides in length. The overhangs are typically present on the 3′ end of each strand, are usually 2 nucleotides long, and are composed of DNA or nucleotide analogs. An siRNA may be formed from two RNA strands that hybridize together, or may alternatively be generated from a longer double-stranded RNA or from a single RNA strand that includes a self-hybridizing portion, such as a short hairpin RNA. Mismatches or unpaired nucleotides may or may not be present in the duplex formed by the two siRNA strands. One strand of an siRNA (the “antisense” or “guide” strand) includes a portion that hybridizes with a target nucleic acid, e.g., an mRNA transcript. In some embodiments, the antisense strand is perfectly complementary to the target over about 15-29 nucleotides, typically between 17-21 nucleotides, e.g., 19 nucleotides, meaning that the siRNA hybridizes to the target transcript without a single mismatch over this length. In some embodiments, one or more mismatches or unpaired nucleotides may be present in a duplex formed between the siRNA strand and the target transcript. In some embodiments, the siRNA may be single stranded.

As used herein, “short hairpin RNA” (shRNA) refers to a nucleic acid molecule comprising at least two complementary portions hybridized or capable of hybridizing to form a duplex structure sufficiently long to mediate RNAi (in some embodiments between 15-29 nucleotides in length), and at least one single-stranded portion, in some embodiments between approximately 1 and 10 nucleotides in length that forms a loop connecting the ends of the two sequences that form the duplex. The structure may further comprise an overhang. The duplex formed by hybridization of self-complementary portions of the shRNA has similar properties to those of siRNAs and, as described elsewhere herein, shRNAs are processed into siRNAs by the conserved cellular RNAi machinery. Thus, shRNAs are precursors of siRNAs and are similarly capable of inhibiting expression of a target transcript. As is the case for siRNA, an shRNA includes a portion that hybridizes with a target nucleic acid, e.g., an mRNA transcript and is, in some embodiments, perfectly complementary to the target over about 15-29 nucleotides, typically between 17-21 nucleotides, e.g., 19 nucleotides. However, in some embodiments, one or more mismatches or unpaired nucleotides may be present in a duplex formed between the shRNA strand and the target transcript. The shRNAs described herein can be useful in implementing gene silencing. In some embodiments, the RNAi structures disclosed herein when compared to duplexes having lengths that are similar or equivalent to the length of the stem of the hairpin in some instances are advantageous, due to the fact that the shRNAs described herein can be more efficient in RNA interference and less likely to induce cellular stress and/or toxicity. Additionally, the phrase “small hairpin RNA” and the term “shRNA” include nucleic acids that also contain moieties other than ribonucleotide moieties, including, but not limited to, modified nucleotides, modified internucleotide linkages, non-nucleotides, deoxynucleotides and analogs of the nucleotides mentioned thereof.

An RNAi is considered to be “targeted” to a transcript and to the gene that encodes the transcript if (1) the RNAi comprises a portion, e.g., a strand, that is at least approximately 80%, approximately 85%, approximately 90%, approximately 91%, approximately 92%, approximately 93%, approximately 94%, approximately 95%, approximately 96%, approximately 97%, approximately 98%, approximately 99%, or approximately 100% complementary to the transcript over a region about 15-29 nucleotides in length, e.g., a region at least approximately 15, approximately 17, approximately 18, or approximately 19 nucleotides in length; and/or (2) the Tm of a duplex formed by a stretch of 15 nucleotides of one strand of the RNAi and a 15 nucleotide portion of the transcript, under conditions (excluding temperature) typically found within the cytoplasm or nucleus of mammalian cells is no more than approximately 15° C. lower or no more than approximately 10° C. lower, than the Tm of a duplex that would be formed by the same 15 nucleotides of the RNAi and its exact complement; and/or (3) the stability of the transcript is reduced in the presence of the RNAi as compared with its absence. An RNAi targeted to a transcript is also considered targeted to the gene that encodes and directs synthesis of the transcript. A target region is a region of a target transcript that hybridizes with an antisense strand of an RNAi.

A “stringent hybridization” and “stringent hybridization wash conditions” in the context of nucleic acid hybridization are sequence dependent, and are different under different experimental parameters. Stringent hybridization conditions can include, e.g., hybridization in a buffer comprising 50% formamide, 5×SSC, and 1% SDS at 42° C., or hybridization in a buffer comprising 5×SSC and 1% SDS at 65° C. both with a wash of 0.2×SSC and 0.1% SDS at 65° C. Other stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPC-4, 7% sodium dodecyl sulfate (SDS), 1 mM EDTA at 65° C., and washing in 0.1×SSC/0.1% SDS at 68° C. can be employed. Additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency. Wash conditions may include, e.g. a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos).

As used herein, “expression vector” refers to a nucleic acid construct, generated recombinantly or synthetically, bearing a series of specified nucleic acid elements that enable transcription of a particular gene in a host cell. In some embodiments, gene expression is placed under the control of certain regulatory elements, such as constitutive or inducible promoters.

As used herein, “operably linked” refers to the connection between regulatory elements and a gene or its coding region. That is, gene expression is typically placed under the control of certain regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element.

The RNA induced silencing complex (RISC) refers to a multiprotein complex, specifically a ribonucleoprotein, which incorporates one strand of a single-stranded RNA (ssRNA) fragment, such as microRNA (miRNA), or double-stranded small interfering RNA (siRNA). The single strand acts as a template for RISC to recognize complementary messenger RNA (mRNA) transcript. Once found, one of the proteins in RISC, called Argonaute, activates and cleaves the mRNA. This process is called RNA interference.

As used herein, the term “small molecule” refers to a non-nucleotidyl, distinct organic compound with a molecular weight markedly lower (e.g., less than or equal to about: 900 Daltons, 1500 Daltons, 2000 Daltons, or ranges including and/or spanning the aforementioned values) compared to the molecular weight of biomolecules. The average size of a small molecule is on the order of less than or equal to about: 1 nm, 2 nm, 3 nm, or ranges including and/or spanning the aforementioned values. Many pharmaceutical drugs are small molecules that may help regulate biological processes.

For any particular non-nucleotidyl compound disclosed herein, the general structure or name presented is also intended to encompass all structural isomers, conformational isomers, and stereoisomers that may arise from a particular set of substituents, unless indicated otherwise. Thus, a general reference to a compound includes all structural isomers unless explicitly indicated otherwise; e.g., a general reference to pentane includes n-pentane, 2-methyl-butane, and 2,2-dimethylpropane while a general reference to a butyl group includes an n-butyl group, a sec-butyl group, an iso-butyl group, and a tert-butyl group. Additionally, the reference to a general structure or name encompasses all enantiomers, diastereomers, and other optical isomers whether in enantiomeric or racemic forms, as well as mixtures of stereoisomers, as the context permits or requires. For any particular formula or name that is presented, any general formula or name presented also encompasses all conformational isomers, regioisomers, and stereoisomers that may arise from a particular set of substituents.

Whenever a group is described as being “optionally substituted” that group may be unsubstituted or substituted with one or more of the indicated substituents. Likewise, when a group is described as being “unsubstituted or substituted” (or “substituted or unsubstituted”) if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), cycloalkyl(alkyl), heteroaryl(alkyl), heterocyclyl(alkyl), hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, nitro, sulfenyl, sulfinyl, sulfonyl, haloalkyl, haloalkoxy, an amino, a mono-substituted amine group, a di-substituted amine group, a mono-substituted amine(alkyl), a di-substituted amine(alkyl), a diamino-group, a polyamino, a diether-group, and a polyether-.

As used herein, “Cₐ to C_{b}” in which “a” and “b” are integers refer to the number of carbon atoms in a group. The indicated group can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated, the broadest range described in these definitions is to be assumed.

If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a}R^{b} group are indicated to be “taken together,” it means that they are covalently bonded to one another to form a ring:

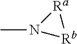

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms, although the present definition also covers the occurrence of the term “alkyl” where no numerical range is designated). The “alkyl” group may also be a medium size alkyl having 1 to 12 carbon atoms. The “alkyl” group could also be a lower alkyl having 1 to 6 carbon atoms. An alkyl group may be substituted or unsubstituted. By way of example only, “C₁-C₅ alkyl” indicates that there are one to five carbon atoms in the alkyl chain, i.e., the alkyl chain is selected from methyl, ethyl, n-propyl, isopropyl, n-butyl, isobutyl, tert-butyl, pentyl (branched and straight-chained), etc. Typical alkyl groups include, but are in no way limited to, methyl, ethyl, propyl, isopropyl, butyl, isobutyl, tertiary butyl, pentyl and hexyl.

As used herein, the term “alkylene” refers to a bivalent fully saturated straight chain aliphatic hydrocarbon group. Examples of alkylene groups include, but are not limited to, methylene, ethylene, propylene, butylene, pentylene, hexylene, heptylene and octylene. An alkylene group may be represented by [symbol] , followed by the number of carbon atoms, followed by a “*”. For example,

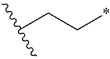 [SMILES: *CC*]

to represent ethylene. The alkylene group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkylene” where no numerical range is designated). The alkylene group may also be a medium size alkyl having 1 to 12 carbon atoms. The alkylene group could also be a lower alkyl having 1 to 6 carbon atoms. An alkylene group may be substituted or unsubstituted. For example, a lower alkylene group can be substituted by replacing one or more hydrogen of the lower alkylene group and/or by substituting both hydrogens on the same carbon with a C₃₋₆ monocyclic cycloalkyl group (e.g.

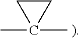

The term “alkenyl” used herein refers to a monovalent straight or branched chain radical of from two to twenty carbon atoms containing a carbon double bond(s) including, but not limited to, 1-propenyl, 2-propenyl, 2-methyl-1-propenyl, 1-butenyl, 2-butenyl and the like. An alkenyl group may be unsubstituted or substituted.

The term “alkynyl” used herein refers to a monovalent straight or branched chain radical of from two to twenty carbon atoms containing a carbon triple bond(s) including, but not limited to, 1-propynyl, 1-butynyl, 2-butynyl and the like. An alkynyl group may be unsubstituted or substituted.

As used herein, “cycloalkyl” refers to a completely saturated (no double or triple bonds) mono- or multi-cyclic (such as bicyclic) hydrocarbon ring system. When composed of two or more rings, the rings may be joined together in a fused, bridged or spiro fashion. As used herein, the term “fused” refers to two rings which have two atoms and one bond in common. As used herein, the term “bridged cycloalkyl” refers to compounds wherein the cycloalkyl contains a linkage of one or more atoms connecting non-adjacent atoms. As used herein, the term “spiro” refers to two rings which have one atom in common and the two rings are not linked by a bridge. Cycloalkyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). A cycloalkyl group may be unsubstituted or substituted. Examples of mono-cycloalkyl groups include, but are in no way limited to, cyclopropyl, cyclobutyl, cyclopentyl, cyclohexyl, cycloheptyl and cyclooctyl. Examples of fused cycloalkyl groups are decahydronaphthalenyl, dodecahydro-1H-phenalenyl and tetradecahydroanthracenyl; examples of bridged cycloalkyl groups are bicyclo[1.1.1]pentyl, adamantanyl and norbornanyl; and examples of spiro cycloalkyl groups include spiro[3.3]heptane and spiro[4.5]decane.

As used herein, “cycloalkenyl” refers to a mono- or multi-cyclic (such as bicyclic) hydrocarbon ring system that contains one or more double bonds in at least one ring; although, if there is more than one, the double bonds cannot form a fully delocalized pi-electron system throughout all the rings (otherwise the group would be “aryl,” as defined herein). Cycloalkenyl groups can contain 3 to 10 atoms in the ring(s), 3 to 8 atoms in the ring(s) or 3 to 6 atoms in the ring(s). When composed of two or more rings, the rings may be connected together in a fused, bridged or spiro fashion. A cycloalkenyl group may be unsubstituted or substituted.

As used herein, a “cycloalkylene group” refers to a group derived by removing two hydrogen atoms from a cycloalkane, at least one of which is a ring carbon. Thus, a “cycloalkylene group” includes both a group derived from a cycloalkane in which two hydrogen atoms are formally removed from the same ring carbon, a group derived from a cycloalkane in which two hydrogen atoms are formally removed from two different ring carbons, and a group derived from a cycloalkane in which a first hydrogen atom is formally removed from a ring carbon and a second hydrogen atom is formally removed from a carbon atom that is not a ring carbon. A “cycloalkane group” refers to a generalized group formed by removing one or more hydrogen atoms (as necessary for the particular group and at least one of which is a ring carbon) from a cycloalkane. It should be noted that according to the definitions provided herein, general cycloalkane groups (including cycloalkyl groups and cycloalkylene groups) include those having zero, one, or more than one hydrocarbyl substituent groups attached to a cycloalkane ring carbon atom (e.g. a methylcyclopropyl group) and is member of the group of hydrocarbon groups. However, when referring to a cycloalkane group having a specified number of cycloalkane ring carbon atoms (e.g. cyclopentane group or cyclohexane group, among others), the base name of the cycloalkane group having a defined number of cycloalkane ring carbon atoms refers to the unsubstituted cycloalkane group (including having no hydrocarbyl groups located on cycloalkane group ring carbon atom). Consequently, a substituted cycloalkane group having a specified number of ring carbon atoms (e.g. substituted cyclopentane or substituted cyclohexane, among others) refers to the respective group having one or more substituent groups (including halogens, hydrocarbyl groups, or hydrocarboxy groups, among other substituent groups) attached to a cycloalkane group ring carbon atom. When the substituted cycloalkane group having a defined number of cycloalkane ring carbon atoms is a member of the group of hydrocarbon groups (or a member of the general group of cycloalkane groups), each substituent of the substituted cycloalkane group having a defined number of cycloalkane ring carbon atoms is limited to hydrocarbyl substituent group. One can readily discern and select general groups, specific groups, and/or individual substituted cycloalkane group(s) having a specific number of ring carbons atoms which can be utilized as member of the hydrocarbon group (or a member of the general group of cycloalkane groups).

As used herein, “aryl” refers to a carbocyclic (all carbon) monocyclic or multicyclic (such as bicyclic) aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group or a C₆ aryl group. Examples of aryl groups include, but are not limited to, benzene, naphthalene and azulene. An aryl group may be substituted or unsubstituted. As used herein, “heteroaryl” refers to a monocyclic or multicyclic (such as bicyclic) aromatic ring system (a ring system with fully delocalized pi-electron system) that contain(s) one or more heteroatoms (for example, 1, 2 or 3 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s), such as nine carbon atoms and one heteroatom; eight carbon atoms and two heteroatoms; seven carbon atoms and three heteroatoms; eight carbon atoms and one heteroatom; seven carbon atoms and two heteroatoms; six carbon atoms and three heteroatoms; five carbon atoms and four heteroatoms; five carbon atoms and one heteroatom; four carbon atoms and two heteroatoms; three carbon atoms and three heteroatoms; four carbon atoms and one heteroatom; three carbon atoms and two heteroatoms; or two carbon atoms and three heteroatoms. Furthermore, the term “heteroaryl” includes fused ring systems where two rings, such as at least one aryl ring and at least one heteroaryl ring or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused, bridged or spiro fashion. As used herein, the term “fused” refers to two rings which have two atoms and one bond in common. As used herein, the term “bridged heterocyclyl” or “bridged heteroalicyclyl” refers to compounds wherein the heterocyclyl or heteroalicyclyl contains a linkage of one or more atoms connecting non-adjacent atoms. As used herein, the term “spiro” refers to two rings which have one atom in common and the two rings are not linked by a bridge. Heterocyclyl and heteroalicyclyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). For example, five carbon atoms and one heteroatom; four carbon atoms and two heteroatoms; three carbon atoms and three heteroatoms; four carbon atoms and one heteroatom; three carbon atoms and two heteroatoms; two carbon atoms and three heteroatoms; one carbon atom and four heteroatoms; three carbon atoms and one heteroatom; or two carbon atoms and one heteroatom. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, azepane, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and/or 3,4-methylenedioxyphenyl). Examples of spiro heterocyclyl groups include 2-azaspiro[3.3]heptane, 2-oxaspiro[3.3]heptane, 2-oxa-6-azaspiro[3.3]heptane, 2,6-diazaspiro[3.3]heptane, 2-oxaspiro[3.4]octane and 2-azaspiro[3.4]octane.

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, 2-phenylalkyl, 3-phenylalkyl and naphthylalkyl.

As used herein, “cycloalkyl(alkyl)” refer to an cycloalkyl group connected, as a substituent, via a lower alkylene group. The lower alkylene and cycloalkyl group of a cycloalkyl(alkyl) may be substituted or unsubstituted.

As used herein, “heteroaralkyl” and “heteroaryl(alkyl)” refer to a heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl and imidazolylalkyl and their benzo-fused analogs.

A “heteroalicyclyl(alkyl)” and “heterocyclyl(alkyl)” refer to a heterocyclic or a heteroalicyclic group connected, as a substituent, via a lower alkylene group. The lower alkylene and heterocyclyl of a (heteroalicyclyl)alkyl may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

As used herein, the term “hydroxy” refers to a —OH group.

As used herein, “alkoxy” refers to the Formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, “acyl” refers to a hydrogen, alkyl, alkenyl, alkynyl, aryl, heteroaryl, heterocyclyl, aryl(alkyl), heteroaryl(alkyl) and heterocyclyl(alkyl) connected, as substituents, via a carbonyl group. Examples include formyl, acetyl, propanoyl, benzoyl and acryl. An acyl may be substituted or unsubstituted.

As used herein, a “cyano” group refers to a “—CN” group.

The term “halogen atom” or “halogen” as used herein, means any one of the radio-stable atoms of column 7 of the Periodic Table of the Elements, such as, fluorine, chlorine, bromine and iodine.

A “thiocarbonyl” group refers to a “—C(═S)R” group in which R can be the same as defined with respect to O-carboxy. A thiocarbonyl may be substituted or unsubstituted. An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-carbamyl may be substituted or unsubstituted.

An “N-carbamyl” group refers to an “ROC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-carbamyl may be substituted or unsubstituted.

An “O-thiocarbamyl” group refers to a “—OC(═S)—N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A C-amido may be substituted or unsubstituted.

An “N-amido” group refers to a “RC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-amido may be substituted or unsubstituted.

An “S-sulfonamido” group refers to a “—SO₂N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An S-sulfonamido may be substituted or unsubstituted.

An “N-sulfonamido” group refers to a “RSO₂N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-sulfonamido may be substituted or unsubstituted.

An “O-carboxy” group refers to a “RC(═O)O—” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “nitro” group refers to an “—NO₂” group.

A “sulfenyl” group refers to an “—SR” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

As used herein, “haloalkyl” refers to an alkyl group in which one or more of the hydrogen atoms are replaced by a halogen (e.g., mono-haloalkyl, di-haloalkyl, tri-haloalkyl and polyhaloalkyl). Such groups include but are not limited to, chloromethyl, fluoromethyl, difluoromethyl, trifluoromethyl, 1-chloro-2-fluoromethyl, 2-fluoroisobutyl and pentafluoroethyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

The terms “amino” and “unsubstituted amino” as used herein refer to a —NH₂ group.

A “mono-substituted amine” group refers to a “—NHR_{A}” group in which R_{A} can be an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. The R_{A} may be substituted or unsubstituted. A mono-substituted amine group can include, for example, a mono-alkylamine group, a mono-C₁-C₆ alkylamine group, a mono-arylamine group, a mono-C₆-C₁₀ arylamine group and the like. Examples of mono-substituted amine groups include, but are not limited to, —NH(methyl), —NH(phenyl) and the like.

A “di-substituted amine” group refers to a “—NR_{A}R_{B}” group in which R_{A} and R_{B} can be independently an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. R_{A} and R_{B} can independently be substituted or unsubstituted. A di-substituted amine group can include, for example, a di-alkylamine group, a di-C₁-C₆ alkylamine group, a di-arylamine group, a di-C₆-C₁₀ arylamine group and the like. Examples of di-substituted amine groups include, but are not limited to, —N(methyl)₂, —N(phenyl)(methyl), —N(ethyl)(methyl) and the like.

As used herein, “mono-substituted amine(alkyl)” group refers to a mono-substituted amine as provided herein connected, as a substituent, via a lower alkylene group. A mono-substituted amine(alkyl) may be substituted or unsubstituted. A mono-substituted amine(alkyl) group can include, for example, a mono-alkylamine(alkyl) group, a mono-C₁-C₆ alkylamine(C₁-C₆ alkyl) group, a mono-arylamine(alkyl group), a mono-C₆-C₁₀ arylamine(C₁-C₆ alkyl) group and the like. Examples of mono-substituted amine(alkyl) groups include, but are not limited to, —CH₂NH(methyl), —CH₂NH(phenyl), —CH₂CH₂NH(methyl), —CH₂CH₂NH(phenyl) and the like.

As used herein, “di-substituted amine(alkyl)” group refers to a di-substituted amine as provided herein connected, as a substituent, via a lower alkylene group. A di-substituted amine(alkyl) may be substituted or unsubstituted. A di-substituted amine(alkyl) group can include, for example, a dialkylamine(alkyl) group, a di-C₁-C₆ alkylamine(C₁-C₆ alkyl) group, a di-arylamine(alkyl) group, a di-C₆-C₁₀ arylamine(C₁-C₆ alkyl) group and the like. Examples of di-substituted amine(alkyl)groups include, but are not limited to, —CH₂N(methyl)₂, —CH₂N(phenyl)(methyl), —CH₂N(ethyl)(methyl), —CH₂CH₂N(methyl)₂, —CH₂CH₂N(phenyl)(methyl), —NCH₂CH₂(ethyl)(methyl) and the like.

As used herein, the term “diamino-” denotes an a “—N(R_{A})R_{B}—N(R_{C})(R_{D})” group in which R_{A}, R_{C}, and R_{D} can be independently a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein, and wherein R_{B} connects the two “N” groups and can be (independently of R_{A}, R_{C}, and R_{D}) a substituted or unsubstituted alkylene group. R_{A}, R_{B}, R_{C}, and R_{D} can independently further be substituted or unsubstituted.

As used herein, the term “polyamino” denotes a “—(N(R_{A})R_{B}—)ₙ—N(R_{C})(R_{D})”. For illustration, the term polyamino can comprise —N(R_{A})alkyl-N(R_{A})alkyl-N(R_{A})alkyl-N(R_{A})alkyl-H. In some embodiments, the alkyl of the polyamino is as disclosed elsewhere herein. While this example has only 4 repeat units, the term “polyamino” may consist of 1, 2, 3, 4, 5, 6, 7, 8, 9, or 10 repeat units. R_{A}, R_{C}, and R_{D} can be independently a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein, and wherein R_{B} connects the two “N” groups and can be (independently of R_{A}, R_{C}, and R_{D}) a substituted or unsubstituted alkylene group. R_{A}, R_{C}, and R_{D} can independently further be substituted or unsubstituted. As noted here, the polyamino comprises amine groups with intervening alkyl groups (where alkyl is as defined elsewhere herein).

As used herein, the term “diether-” denotes an a “—OR_{B}O—R_{A}” group in which R_{A} can be a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein, and wherein R_{B} connects the two “O” groups and can be a substituted or unsubstituted alkylene group. R_{A} can independently further be substituted or unsubstituted.

As used herein, the term “polyether” denotes a repeating —(OR_{B}—)ₙOR_{A} group. For illustration, the term polyether can comprise —Oalkyl-Oalkyl-Oalkyl-Oalkyl-OR_{A}. In some embodiments, the alkyl of the polyether is as disclosed elsewhere herein. While this example has only 4 repeat units, the term “polyether” may consist of 1, 2, 3, 4, 5, 6, 7, 8, 9, or 10 repeat units. R_{A} can be a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. R_{B} can be a substituted or unsubstituted alkylene group. R_{A} can independently further be substituted or unsubstituted. As noted here, the polyether comprises ether groups with intervening alkyl groups (where alkyl is as defined elsewhere herein and can be optionally substituted).

Where the number of substituents is not specified (e.g. haloalkyl), there may be one or more substituents present. For example, “haloalkyl” may include one or more of the same or different halogens. As another example, “C₁-C₃ alkoxyphenyl” may include one or more of the same or different alkoxy groups containing one, two or three atoms.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

The term “organyl group” is used herein in accordance with the definition specified by IUPAC: an organic substituent group, regardless of functional type, having one free valence at a carbon atom. Similarly, an “organylene group” refers to an organic group, regardless of functional type, derived by removing two hydrogen atoms from an organic compound, either two hydrogen atoms from one carbon atom or one hydrogen atom from each of two different carbon atoms. An “organic group” refers to a generalized group formed by removing one or more hydrogen atoms from carbon atoms of an organic compound. Thus, an “organyl group,” an “organylene group,” and an “organic group” can contain organic functional group(s) and/or atom(s) other than carbon and hydrogen, that is, an organic group can comprise functional groups and/or atoms in addition to carbon and hydrogen. For instance, non-limiting examples of atoms other than carbon and hydrogen include halogens, oxygen, nitrogen, phosphorus, and the like. Non-limiting examples of functional groups include ethers, aldehydes, ketones, esters, sulfides, amines, phosphines, and so forth. In one aspect, the hydrogen atom(s) removed to form the “organyl group,” “organylene group,” or “organic group” may be attached to a carbon atom belonging to a functional group, for example, an acyl group (—C(O)R), a formyl group (—C(O)H), a carboxy group (—C(O)OH), a hydrocarboxycarbonyl group (—C(O)OR), a cyano group (—C≡N), a carbamoyl group (—C(O)NH₂), an N-hydrocarbylcarbamoyl group (—C(O)NHR), or N,N′-dihydrocarbylcarbamoyl group (—C(O)NR₂), among other possibilities. In another aspect, the hydrogen atom(s) removed to form the “organyl group,” “organylene group,” or “organic group” may be attached to a carbon atom not belonging to, and remote from, a functional group, for example, CH₂C(O)CH₃, CH₂NR₂, and the like. An “organyl group,” “organylene group,” or “organic group” may be aliphatic, inclusive of being cyclic or acyclic, or may be aromatic. “Organyl groups,” “organylene groups,” and “organic groups” also encompass heteroatom-containing rings, heteroatom-containing ring systems, heteroaromatic rings, and heteroaromatic ring systems. “Organyl groups,” “organylene groups,” and “organic groups” may be linear or branched unless otherwise specified. Finally, it is noted that the “organyl group,” “organylene group,” or “organic group” definitions include “hydrocarbyl group,” “hydrocarbylene group,” “hydrocarbon group,” respectively, and “alkyl group,” “alkylene group,” and “alkane group,” respectively, as members.

The term “hydrocarbyl group” is used herein in accordance with the definition specified by IUPAC: a univalent group formed by removing a hydrogen atom from a hydrocarbon. Non-limiting examples of hydrocarbyl groups include ethyl, phenyl, tolyl, propenyl, and the like. Similarly, a “hydrocarbylene group” refers to a group formed by removing two hydrogen atoms from a hydrocarbon, either two hydrogen atoms from one carbon atom or one hydrogen atom from each of two different carbon atoms. Therefore, in accordance with the terminology used herein, a “hydrocarbon group” refers to a generalized group formed by removing one or more hydrogen atoms (as necessary for the particular group) from a hydrocarbon. A “hydrocarbyl group,” “hydrocarbylene group,” and “hydrocarbon group” can be acyclic or cyclic groups, and/or may be linear or branched. A “hydrocarbyl group,” “hydrocarbylene group,” and “hydrocarbon group” can include rings, ring systems, aromatic rings, and aromatic ring systems, which contain only carbon and hydrogen. “Hydrocarbyl groups,” “hydrocarbylene groups,” and “hydrocarbon groups” include, by way of example, aryl, arylene, arene, alkyl, alkylene, alkane, cycloalkyl, cycloalkylene, cycloalkane, aralkyl, aralkylene, and aralkane groups, among other groups, as members.

An aromatic compound is a compound containing a cyclically conjugated double bond system that follows the Hückel (4n+2) rule and contains (4n+2) pi-electrons, where n is an integer from 1 to 5. Aromatic compounds include “arenes” (hydrocarbon aromatic compounds) and “heteroarenes,” also termed “hetarenes” (heteroaromatic compounds formally derived from arenes by replacement of one or more methine (—C═) carbon atoms of the cyclically conjugated double bond system with trivalent or divalent heteroatoms, in such a way as to maintain the continuous pi-electron system characteristic of an aromatic system and a number of out-of-plane pi-electrons corresponding to the Hückel rule (4n+2). While arene compounds and heteroarene compounds are mutually exclusive members of the group of aromatic compounds, a compound that has both an arene group and a heteroarene group are generally considered a heteroarene compound. Aromatic compounds, arenes, and heteroarenes can be monocyclic (e.g., benzene, toluene, furan, pyridine, methylpyridine) or polycyclic unless otherwise specified. Polycyclic aromatic compounds, arenes, and heteroarenes, include, unless otherwise specified, compounds wherein the aromatic rings can be fused (e.g., naphthalene, benzofuran, and indole), compounds where the aromatic groups can be separate and joined by a bond (e.g., biphenyl or 4-phenylpyridine), or compounds where the aromatic groups are joined by a group containing linking atoms (e.g., carbon—the methylene group in diphenylmethane; oxygen—diphenyl ether; nitrogen—triphenyl amine; among others linking groups). As disclosed herein, the term “substituted” can be used to describe an aromatic group, arene, or heteroarene wherein a non-hydrogen moiety formally replaces a hydrogen in the compound, and is intended to be non-limiting.

An “aromatic group” refers to a generalized group formed by removing one or more hydrogen atoms (as necessary for the particular group and at least one of which is an aromatic ring carbon atom) from an aromatic compound. For a univalent “aromatic group,” the removed hydrogen atom must be from an aromatic ring carbon. For an “aromatic group” formed by removing more than one hydrogen atom from an aromatic compound, at least one hydrogen atom must be from an aromatic hydrocarbon ring carbon. Additionally, an “aromatic group” may have hydrogen atoms removed from the same ring of an aromatic ring or ring system (e.g., phen-1,4-ylene, pyridin-2,3-ylene, naphth-1,2-ylene, and benzofuran-2,3-ylene), hydrogen atoms removed from two different rings of a ring system (e.g., naphth-1,8-ylene and benzofuran-2,7-ylene), or hydrogen atoms removed from two isolated aromatic rings or ring systems (e.g., bis(phen-4-ylene)methane).

An arene is aromatic hydrocarbon, with or without side chains (e.g. benzene, toluene, or xylene, among others. An “aryl group” is a group derived from the formal removal of a hydrogen atom from an aromatic ring carbon of an arene. It should be noted that the arene may contain a single aromatic hydrocarbon ring (e.g., benzene, or toluene), contain fused aromatic rings (e.g., naphthalene or anthracene), and contain one or more isolated aromatic rings covalently linked via a bond (e.g., biphenyl) or non-aromatic hydrocarbon group(s) (e.g., diphenylmethane). One example of an “aryl group” is ortho-tolyl (o-tolyl), the structure of which is shown here.

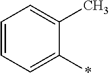 [SMILES: *c1ccccc1C]

A heteroarene is aromatic compound, with or without side chains, having a heteroatom within the aromatic ring or aromatic ring system (e.g. pyridene, indole, or benzofuran, among others). A “heteroaryl group” is a class of “heterocyclyl group” and is a univalent group formed by removing a hydrogen atom from a heteroaromatic ring or ring system carbon atom of a heteroarene compound.

There exists a need for compositions and methods that can intervene in the progressive breakdown of tissue function and may repair or stimulate aging or dysfunctional cells and tissues. Disclosed herein are methods, agents, and compositions for preventing or treating age-related dysfunction and/or dysfunction that is not related to aging but that manifests biological and physiological outcomes that are similar or the same as those found in aging cells. In some embodiments, the methods disclosed herein include administering agents that reduce the expression of a paired box 5 (PAX5) gene and/or reducing expression of a protein phosphatase 1F enzyme (PPM1F) gene. In some embodiments, this expression is reduced in a cell using one or more interfering RNAs (RNAi(s)), small molecule drug compounds, cells treated with such agents, or combinations thereof. In some embodiments, the methods include cell therapies. In some embodiments, cells are manipulated and to provide therapeutic cells which can be implanted in the body to achieve one or more therapeutic effects. In some embodiments, prepared therapeutic cells are exposed to patient cells to provide target cells. In some embodiments, target cells can be manipulated to provide additional therapeutic cells and/or can be reintroduced to the patient to achieve a therapeutic effect. In some embodiments, an RNAi that reduces the expression of PAX5, PPM1F, both, other genes as disclosed elsewhere herein, and/or genes encoding proteins disclosed herein is referred to as senescence-agent disruptors (SAD). Some embodiments pertain to methods of treating PAX5 and/or PPM1F gene mediated conditions in a patient.

### PAX5 and PPM1F for Achieving Therapeutic Effect

B-cell lineage specific activator protein (BSAP) is expressed at early, but not late, stages of B-cell differentiation. BSAP is a nuclear protein in the paired-box (PAX) containing family of transcription factors involved in control of organ development and tissue differentiation. BSAP is encoded by the PAX5 gene which is primarily expressed in B lymphocytes and B-cell lymphomas, with additional expression in the developing central nervous system. As shown in FIG. 20, which illustrates the PAX5 signaling cascade, PAX5 function influences a number of cellular processes, including cell growth, DNA repair, apoptosis, and tumor growth (e.g., hepatocellular carcinoma or “HCC”). As shown, PAX5 also influences the p53 signaling pathway. Among other things, PAX5 up-regulates the effectors of p53-dependent apoptosis, including ligands responsible for extracellular death (e.g., TNF, Fas-L, adaptor protein LRDD, p53 family members p63 and p73, Bcl2 family members Noxa and PUMA). These ligands induce caspase dependent cellular apoptosis. PAX5 may increase p53 transcription target genes p21, RPRM, and PCBp4 resulting in cell growth arrest. PAX5 induced p53-dependent DNA repair occurs through GADD45, which protects against tumorigenesis through maintaining genomic stability. Some embodiments disclosed herein influence one or more of the molecules and ligands (e.g., through interfering RNA mechanism, etc.) to interrupt the normal PAX5 cascade.

A feature of the PAX gene family is a novel, highly conserved DNA-binding domain, known as the paired box. The PAX proteins (e.g., BSAP) are important regulators in early development, and alterations in the expression of their genes are thought to contribute to neoplastic transformation. Its expression has also been detected in developing CNS and testis, therefore, the PAX5 gene product may not only play an important role in B-cell differentiation, but also in neural development and spermatogenesis. Some embodiments disclosed herein influence the PAX5 gene or protein (e.g., through interfering RNA mechanism, through inhibition of the protein, etc.) to interrupt PAX5 function.

The protein phosphatase 1F enzyme (PP1F) is a member of the PP2C family of Ser/Thr protein phosphatases which are known to be negative regulators of cell stress response pathways. This phosphatase can interact with Rho guanine nucleotide exchange factors (PIX), and thus block the effects of p21-activated kinase 1 (PAK), a protein kinase mediating biological effects downstream of Rho GTPases. Calcium/calmodulin-dependent protein kinase II gamma (CAMK2G/CAMK-II) is found to be one of the substrates of this phosphatase. The overexpression of this phosphatase or CAMK2G has been shown to mediate caspase-dependent apoptosis. An alternatively spliced transcript variant has been identified, but its full-length nature has not been determined. Protein phosphatase 1F enzyme is encoded by the PPM1F gene. Some embodiments disclosed herein influence one or more of the molecules and ligands (e.g., through interfering RNA mechanism, etc.) to interrupt the normal PPM1F cascade.

As shown in FIG. 21, multiple strategies can be used to interfere with the PAX5 gene/protein and/or the PPM1F gene/protein. These approaches can involve using one or more of interfering RNA(s) (RNAi(s)), compounds that inhibit the PAX5 gene and/or the PPM1F gene, and/or using combinations of RNAi(s) and inhibitor compounds together. In other words, in some embodiments, the method of reducing expression of the PAX5 gene and/or reducing expression of the PPM1F gene in a cell comprises contacting the cell with one or more of interfering RNA(s) (RNAi(s)), compounds that inhibit the PAX5 gene and/or the PPM1F gene, and/or using combinations of RNAi(s) and inhibitor compounds together.

Additionally, in some embodiments, interference with one or more of the PAX5 or PPM1F genes and/or proteins can be achieved using a variety of strategies. For example, in some embodiments, as illustrated in FIGS. 22A-B, active agents can act upon cells ex vivo (FIG. 22A) that are then introduced in vivo (FIG. 22B). Alternatively, active agents can be introduced to a patient (e.g., systemically or locally) to act upon cells in vivo to elicit a therapeutic effect (FIG. 22C).

For example, in some embodiments, as shown in FIG. 22A, PAX5 and/or PPM1F interference and/or inhibition can be performed on a cell 101 that is isolated from a patient 100. As shown, one or more RNAi(s) for PAX5, compounds that inhibit the PAX5 gene, and/or using combinations thereof (collectively interfering agents 102) can be used to treat the cell to inhibit the PAX5 gene, causing the down regulation of the PAX5 protein and affording a cell with improved cellular function 103. As shown, one or more RNAi(s) for PPM1F, compounds that inhibit the PPM1F gene, and/or using combinations thereof (collectively interfering agents 104) can be used to treat the cell to inhibit the PPM1F gene, causing the down regulation of the PPM1F protein and affording a cell with improved cellular function 105. As shown, one or more RNAi(s) for PAX5 and/or PPM1F, compounds that inhibit the PAX5 and/or PPM1F gene, and/or using combinations thereof (collectively interfering agents 106) can be used to treat the cell to inhibit the PAX5 and PPM1F gene, causing the down regulation of the PAX5 and PPM1F protein and affording a cell with improved cellular function 107.

As shown in FIG. 22B, each of these cells with improved cellular function 103, 105, 107 (e.g., a therapeutic cell) can be reintroduced 108, 109, 110 to the patient, resulting in a treated patient 111. In some embodiments, once in the body, the therapeutic cell can influence other cells in vivo to cause a broader therapeutic effect that can be long lasting.

Alternatively, in some embodiments, as shown in FIG. 22C, PAX5 and/or PPM1F interference and/or inhibition can be performed on a cell that is in a patient 200. As shown, one or more RNAi(s) for PAX5, compounds that inhibit the PAX5 gene, and/or using combinations thereof (collectively interfering agents 202) can be used to treat the in vivo cell to inhibit the PAX5 gene, causing the down regulation of the PAX5 protein and affording a cell with improved cellular function 203. As shown, one or more RNAi(s) for PPM1F, compounds that inhibit the PPM1F gene, and/or using combinations thereof (collectively interfering agents 204) can be used to treat the cell in vivo to inhibit the PPM1F gene, causing the down regulation of the PPM1F protein and affording a cell with improved cellular function 205. As shown, one or more RNAi(s) for PAX5 and/or PPM1F, compounds that inhibit the PAX5 and/or PPM1F gene, and/or using combinations thereof (collectively interfering agents 206) can be used to treat the cell in vivo to inhibit the PAX5 and PPM1F gene, causing the down regulation of the PAX5 and PPM1F protein and affording a cell with improved cellular function 207.

In some embodiments, both the strategies of FIGS. 22A-B and that of FIG. 22C can be used in combination (e.g., to achieve an enhanced effect and/or where one mode of therapy is more suited for a particular pathway than another). In some embodiments, additional potential treatment strategies include those shown in the flow charts of FIGS. 22C-O, which are described in more detail elsewhere herein.

### Interfering RNAs

Some embodiments pertain to an interfering RNA (RNAi) that reduces the expression of a gene or protein. In some embodiments, by reducing the expression of a gene, for example, in a cell, one or more cellular benefits is achieved. In some embodiments, the benefits are related to treating one or more symptoms of aging and/or one or more symptoms of dysfunctional cellular processes. In some embodiments, the RNAi reduces the expression of one or more of the PAX5, PPM1F, and/or CAMK2G genes.

In some embodiments, a reduction of expression includes reducing expression by greater than or at least about: 1%, 5%, 10%, 30%, 40%, 50%, 60%, 70%, 80%, 90%, 95%, 98%, 99%, 99.9%, 100%, or ranges including and/or spanning the aforementioned values. In some embodiments, a reduction of expression includes at least one of of decreasing the amount of native protein synthesized, full length protein synthesized, decreasing the amount of functional protein synthesized, decreasing the amount of functional fragments of protein synthesized, and decreasing the amount of fragments of protein synthesized. Unless otherwise noted, reduced expression will denote a reduction of the expression of the functional protein.

In some embodiments, the RNAi comprises 4 to 50 contiguous nucleotides having a polynucleotide sequence that is at least 80% to 100% complementary to a region of a gene encoding a protein as disclosed elsewhere herein. In some embodiments, the RNAi comprises a polynucleotide sequence comprising 4 to 50 contiguous nucleotides having a polynucleotide sequence that is at least 80% to 100% complementary to a region of a gene of SEQ ID NO:1 (PAX5; Homo sapien—as shown in FIG. 22P1), SEQ ID NO:2 (PAX5; Equus caballus—as shown in FIG. 22P2), SEQ ID NO:3 (PAX5; Canis lupus—as shown in FIG. 22P3), SEQ ID NO:4 (PAX5; Felis catus—as shown in FIG. 22P4), SEQ ID NO:5 (PPM1F; Homo sapien—as shown in FIG. 22P5), SEQ ID NO:6 (PPM1F; Equus caballus—as shown in FIG. 22P6), SEQ ID NO:7 (PPM1F; Canis lupus—as shown in FIG. 22P7), and/or SEQ ID NO:8 (PPM1F; Felis catus—as shown in FIG. 22P8), SEQ ID NO:21 (CAMK2G; Homo sapien—as shown in FIG. 22P21). In some embodiments, the RNAi comprises a polynucleotide sequence comprising 4 to 50 contiguous nucleotides having a polynucleotide sequence that is at least 80% to 100% complementary to a region of a gene of SEQ ID NO:1 or SEQ ID NO:5. Unless otherwise noted herein, reference to the PAX5 and/or the PPM1F gene will denote and/or include the human sequences as shown in FIG. 22P1 and FIG. 22P5, respectively. Unless otherwise noted herein, reference to the CAMK2G gene will denote and/or include the human sequence as shown in FIG. 22P21.

Sequences for various variants of the targets are presented in FIGS. 22P1-P8 and 22P21. Conservation across organisms indicate sections of the sequences that could be used universally as iRNAs, while sections of variation demonstrate sequences that can be specific to various organisms. In some embodiments, interference with the expression of one or more of the genes of SEQ ID NOs: 1-8 and 21, results in a decreased amount of the coinciding proteins of those genes, for example, SEQ ID NOs: 22-30, respectively, being expressed and synthesized.

Some embodiments pertain to a method of reducing expression of a paired box 5 (PAX5) gene and reducing expression of a protein phosphatase 1F enzyme (PPM1F) gene using one or more interfering RNAs (RNAi(s)), compounds, or combinations thereof. While, in some embodiments, the RNAi is administered to a human (and/or to at least one human cell) to treat that human, in other embodiments, the RNAi is administered to, for example, a horse, dog, cat, or other mammal (and/or to at least one cell of the mammal). The nucleic acid sequence for the human PAX5 gene is provided as SEQ ID NO: 1. The nucleic acid sequences for the horse, dog, and cat PAX5 gene are provided as SEQ ID NO: 2, SEQ ID NO: 3, and SEQ ID NO: 4, respectively. The nucleic acid sequence for the human PPM1F gene is provided as SEQ ID NO: 5. The nucleic acid sequences for the horse, dog, and cat PPM1F gene is provided as SEQ ID NO: 6, SEQ ID NO: 7, and SEQ ID NO: 8, respectively.

In some embodiments, as shown in FIGS. 22D and 22E, the method includes selecting or acquiring a patient cell 110. In some embodiments, as shown in FIG. 22C, the cell may be a cell in the body of the patient (for in vivo treatment) or isolated from the patient (for ex vivo and/or in vitro treatment). In some embodiments, as shown, the cell is treated by exposing it to one or more different PAX5 gene RNAi(s) 111′. In some embodiments, as shown, the cell is treated by exposing it to one or more different PPM1F gene RNAi(s) 111″. In some embodiments, as shown, the RNAi(s) are allowed to act on the cell for a period of time 112. In some embodiments, as shown, this results in a target cell 113. In some embodiments, as shown in FIG. 22E, the cell can be reintroduced to the patient (e.g., where it was initially isolated from the patient). In some embodiments, the entire process is performed in vivo and, therefore, the cell need not be reintroduced to the patient.

Some embodiments pertain to a method of reducing expression of a calcium/calmodulin dependent protein kinase II gamma (CAMK2G) gene. In some embodiments, an RNAi is administered to a human and/or to at least one cell of a human. The nucleic acid sequence for the human CAMK2G gene is provided as SEQ ID NO: 21.

In some embodiments, the RNAi is a short interfering RNA (siRNA), microRNA (miRNA), circular RNAs (circRNAs), short hairpin RNAs (shRNAs), long non-coding RNAs (lncRNAs); piwi-interacting RNAs (piRNA), small nucleolar RNA (snoRNAs), tRNA-derived small RNA (tsRNA), small rDNA-derived RNA (srRNA), or a small nuclear RNA (U-RNA). In some embodiments, the RNAi is an siRNA.

In some embodiments, the number of contiguous nucleotides in the RNAi is less than or equal to about: 200, 150, 100, 50, 40, 30, 25, 20, 10, 4, or ranges including and/or spanning the aforementioned values. In some embodiments, the RNAi comprises about 20 to 30 contiguous nucleotides.

In some embodiments, the RNAi comprises a polynucleotide sequence comprising 4 to 50 contiguous nucleotides having a polynucleotide sequence that is at least 80% to 100% complementary to a region of a gene of any one of SEQ ID NOs:1-8 and 21. In some embodiments, the region of complementarity between the RNAi and a target sequence (e.g., a portion of SEQ ID NOs:1-8 and 21) may be substantially complementary (e.g., there is a sufficient degree of complementarity between the RNAi and a target nucleic acid to so that they specifically hybridize and induce a desired effect). In some embodiments, the RNAi is fully complementary to the target sequence (100% complementary). In some embodiments, the RNAi may include a contiguous nucleotide sequence comprising no more than 5 mismatches (e.g., no more than 1, no more than 2, no more than 3, no more than 4, or no more than 5 mismatches) when hybridizing to a target sequence, such as to the corresponding region of a portion of SEQ ID NOs:1-8 and 21. In some embodiments, the contiguous nucleotide sequence comprises no more than a single mismatch when hybridizing to the target sequence, such as the corresponding region of a portion of SEQ ID NOS:1-8 and 21. In some embodiments, RNAi hybridizes to a complimentary region (e.g., a target region) of any one of SEQ ID NOs:1-8 and 21, thereby interfering with the transcription of that gene. In some embodiments, the RNAi is complementary in one section (as noted above), but has a section with 1, 2, 3, 4, or more nucleotides that are not complementary.

In some embodiments, the RNAi comprises a polynucleotide chain comprising nucleotides that are complementary to polynucleotides that transcribe any one of SEQ ID NOs:1-8 and 21 (e.g., mRNA). The region of complementarity between the RNAi and a target sequence (e.g., a portion of mRNA that transcribes SEQ ID NOs:1-8 and 21) may be substantially complementary (e.g., there is a sufficient degree of complementarity between the RNAi and a target nucleic acid to so that they specifically hybridize and induce a desired effect). In some embodiments, the RNAi is fully complementary to the target sequence (e.g., 100% complementary). In some embodiments, the RNAi may include a contiguous nucleotide sequence comprising no more than 5 mismatches (e.g., no more than 1, no more than 2, no more than 3, no more than 4, or no more than 5 mismatches) when hybridizing to a target sequence, such as to the corresponding region of an mRNA encoding any one of SEQ ID NOs:1-8 and 21. In some embodiments, the contiguous nucleotide sequence comprises no more than a single mismatch when hybridizing to the target sequence, such as the mRNA encoding a region of a portion of SEQ ID NOs:1-8 and 21. In some embodiments, the RNAi hybridizes to a target nucleic acid molecule, such as the mRNA encoding PAX5, PPM1F, or CAMK2G, and comprises a contiguous nucleotide sequence which corresponds to the reverse complement of a nucleotide sequence of any one of SEQ ID NOs:1-8 and 21, or a fragment of any one of SEQ ID NOs:1-8 and 21. As will be appreciated by one of skill in the art, for double stranded RNAi, one sequence will be complementary to the target, while the other strand will be identical to the target. Thus, in some embodiments, any description provided herein regarding a sequence that is complementary to, can also describe a sequence that is the same as (when the hybridized chain is being referenced).

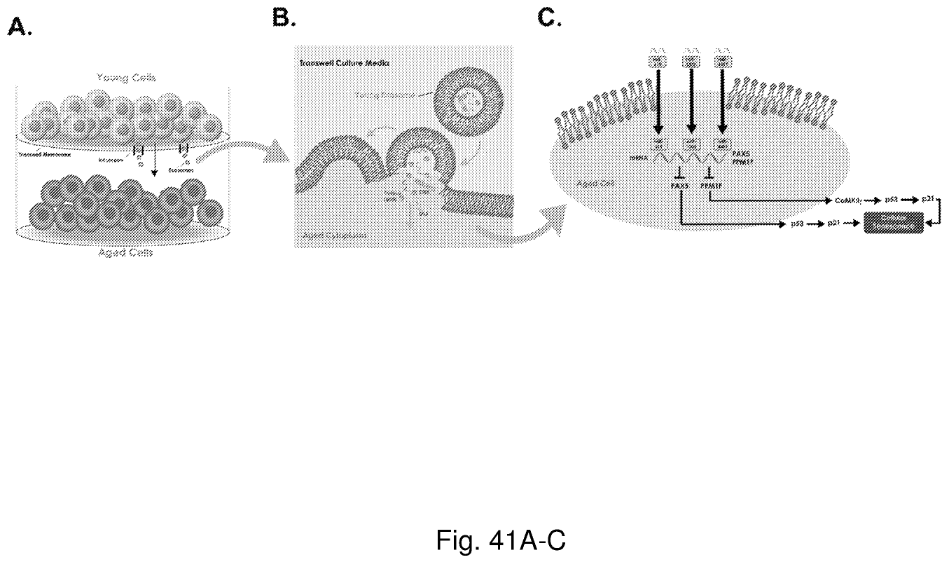

In some embodiments, the RNAi is at least about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, about 99.99%, or about 100% complementary to a target sequence (or ranges including and/or spanning the aforementioned values). In some embodiments, the target sequence is a polynucleotide region of less than or equal to about: 200, 150, 100, 50, 40, 30, 25, 20, 10, or 4 (or ranges including and/or spanning the aforementioned values) nucleotides in length. In some embodiments, the RNAi is prepared synthetically and is not an RNAi that has been isolated from a natural source. In some embodiments, as disclosed elsewhere herein, the target region can include a region of the gene encoding the protein or a region of an mRNA transcribing the gene. In some embodiments, the target region is a region of any one of SEQ ID NOs:1-8 and 21, CAMK2G/CAMK-II, PAK, C21orf62-AS1, CASP14, CATSPER2, DNAH10OS, ELMOD1, GALNT6, HEPN1, LANCL2, LL22NC03-63E9.3, PPTC7, PROSC, RAB3B, RRP7A, SERF1A/SERF1B, SLC35E3, SMIM10, SPRY3, SUMO2, TPP1, TPPP, WBP1L, ZNF33A, ZNF549, a gene encoding any one of the molecules disclosed in FIGS. 20 and 41, or an mRNA transcribing any one of the foregoing. As noted herein, the sequence will be of an appropriate length and composition to allow for the correct hybridization, and can be, for example 10-50 nucleic acids in length. In some embodiments, the hybridization conditions are set to those for ex vivo therapy. In some embodiments, the hybridization is performed in a solution having a concentration of MgCl2 of equal to or less than about: 1 mM, 2.5 mM, 5 mM, 7.5 mM, 10 mM, or ranges including and/or spanning the aforementioned values. In some embodiments, the hybridization is performed in a solution having a temperature of equal to or at least about: 45° C., 50° C., 55° C., 60° C., 65° C., or ranges including and/or spanning the aforementioned values.

In some embodiments, combinations of various RNAi species can be used (e.g., in methods of treating patients and/or cells). In some embodiments, a plurality (e.g., 1, 2, 3, 4, 5, or more) RNAi(s) are administered. In some embodiments, the RNAi or RNAi(s) are isolated prior to use. In some embodiments, the RNAi(s) are synthesized. In some embodiments, the RNAi(s) are synthesized using, for example, PCR. In some embodiments, the members of a combination can be administered substantially simultaneously and/or sequentially. In some embodiments, when administered simultaneously, the RNAi(s) can be part of a composition. In some embodiments, combinations of RNAi species are not used and a single RNAi species can be administered. In some embodiments, the administered RNAi has a sequence that is identical to that of any one of SEQ ID NOs:9-20 (as shown in Table 3 below). In some embodiments, the RNAi or combination of RNAi(s) have sequences that are independently at least about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, about 99.99%, or about 100% identical to any one of SEQ ID NOs:9-20 (or ranges including and/or spanning the aforementioned values). In some embodiments, one or more RNAi(s) having a gene target (e.g., PAX5, PPM1F, CAMK2G, etc.) are used in a method of treating. In some embodiments, the one or more RNAi(s) having a gene target (e.g., PAX5, PPM1F, CAMK2G, etc.) are selected from one or more of SEQ ID NO:9, SEQ ID NO:10, SEQ ID NO:11, SEQ ID NO:12, SEQ ID NO: 13, SEQ ID NO: 14, SEQ ID NO: 15, SEQ ID NO: 16, SEQ ID NO: 17, SEQ ID NO: 18, SEQ ID NO:19, and/or SEQ ID NO:20. In some embodiments, multiple RNAi(s) having a mutual gene target (e.g., PAX5, PPM1F, CAMK2G, etc.) are used. For example, in some embodiments, two or more of SEQ ID NO:9, SEQ ID NO: 10, SEQ ID NO: 11, SEQ ID NO: 15, SEQ ID NO: 16, SEQ ID NO: 17, SEQ ID NO: 18, SEQ ID NO: 19, or SEQ ID NO:20, which each are RNAi(s) for PAX5, are used in combination to interfere with the PAX5 gene. In some embodiments, SEQ ID Nos: 12-20, which each are RNAi(s) for PPM1F, are used in combination to interfere with the PPM1F gene. In some embodiments, SEQ ID NOs: 15, 17, and 19, which each are RNAi(s) for the PAX5 and the PPM1F gene, are used in combination to interfere with those genes. In some embodiments, the nucleic acid is one that hybridizes to a sequence that is complementary to any one or more of the sequences of SEQ ID NOs:9-20 under stringent hybridization conditions.

TABLE 3
Some embodiments of RNAi(s).

| SEQ | | |
|---|---|---|
| ID | | |
| NO: | Sequence | RNAi Type |
| 9 | CCGGUGAUGUAGACAAUAAUUAACA | siRNA |
| 10 | GCAAGAGAGGAAGGUAUUCAGGA | siRNA |
| 11 | GCCUUAAUUCCUUGCAAUAGUCUCTC | siRNA |
| 12 | GUUGAGACCAUGCAGUCAAUGCATT | siRNA |
| 13 | AGACCUUUCCGAAUUCAGGAAGUTG | siRNA |
| 14 | CACCAAGAAGCUAGGUGGUUUCCAG | siRNA |
| 15 | GCUGGGAUUACAGGCAUGAGCC | miRNA (miR-619-5p) |
| 16 | CGCCCACCUCAGCCUCCCAAAAUGC | shRNA (miR-619-5p |
| | UGGGAUUACAGGCAUGAGCCACUGC | stem loop) |
| | GGUCGACCAUGACCUGGACAUGUUU | |
| | GUGCCCAGUACUGUCAGUUUGCAG | |
| 17 | UUUAGAGACGGGGUCUUGCUCU | miRNA (miR-1303) |
| 18 | GGCUGGGCAACAUAGCGAGACCUCA | shRNA (miR-1303 |
| | ACUCUACAAUUUUUUUUUUUUUAAA | stem loop) |
| | UUUUAGAGACGGGGUCUUGCUCUGU | |
| | UGCCAGGCUUU | |
| 19 | CUCCGGGACGGCUGGGC | miRNA (miR-4497) |
| 20 | ACCUCCGGGACGGCUGGGCGCCGGC | shRNA (miR-4497 |
| | GGCCGGGAGAUCCGCGCUUCCUGAA | stem loop) |
| | UCCCGGCCGGCCCGCCCGGCGCCCG | |
| | UCCGCCCGCGGGUC | |

As disclosed elsewhere herein, some embodiments pertain to compositions for reducing expression of genes (e.g., PAX5, PPM1F, CAMK2G, etc.). In some embodiments, the composition comprises one or more of an RNAi, a small molecule inhibitor of a gene, and/or a pharmaceutically excipient, diluent, or carrier. In some embodiments, the composition comprises a single RNAi species or a plurality of different RNAi species. In some embodiments, the composition comprises a single small molecule inhibitor or a plurality of different small molecule inhibitors. In some embodiments, the composition lacks one or more of an RNAi, a small molecule inhibitor of PAX5, PPM1F, CAMK2G, and/or a pharmaceutically excipient, diluent, or carrier.

Some embodiments pertain to one or more RNAi(s) for reducing expression of a PAX5 gene. In some embodiments, as shown in FIGS. 22F and 22G, the method includes selecting or acquiring a patient cell 110. In some embodiments, as shown in FIG. 22C, the cell may be a cell in the body of the patient (for in vivo treatment) or isolated from the patient (for ex vivo and/or in vitro treatment). In some embodiments, as shown, the cell is treated by exposing it to one or more different PAX5 gene RNAi(s) 111′. In some embodiments, as shown, the RNAi(s) are allowed to act on the cell for a period of time 112. In some embodiments, as shown, this results in a target cell 113. In some embodiments, as shown in FIG. 22G, the target cell can be reintroduced to the patient (e.g., where it was initially isolated from the patient). In some embodiments, the entire process is performed in vivo and, therefore, the cell need not be reintroduced to the patient.

In some embodiments, the RNAi(s) for reducing expression of a PAX5 gene comprise any one or more of SEQ ID NO:9, SEQ ID NO:10, SEQ ID NO:11, SEQ ID NO:15, SEQ ID NO:16, SEQ ID NO:17, SEQ ID NO:18, SEQ ID NO:19, or SEQ ID NO:20. In some embodiments, the RNAi(s) for reducing expression of a PAX5 gene is at least about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, about 99.99%, or about 100% identical to any one of SEQ ID NOs:9-11 or 15-20 (or ranges including and/or spanning the aforementioned values). In some embodiments, one, two, or three amino acids of any one of SEQ ID NOs:9-11 or 15-20 can be exchanged with another amino acid (e.g., any one of G, A, S, T, C, V, L, I, M, P, F, Y, W, D, E, N, Q, H, K, R, etc.) to provide different RNAi, so long as the RNAi still hybridizes to its gene target to achieve binding and/or inhibition.

In some embodiments, an RNAi composition for reducing expression of a PAX5 gene is provided. In some embodiments, the composition comprises the one or more RNAi(s). In some embodiments, the composition comprises of any one or more of SEQ ID NO:9, SEQ ID NO:10, SEQ ID NO:11, SEQ ID NO:15, SEQ ID NO:16, SEQ ID NO:17, SEQ ID NO:18, SEQ ID NO:19, or SEQ ID NO:20. In some embodiments, the composition for reducing expression of a PAX5 gene comprises RNAi(s) that are at least about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, about 99.99%, or about 100% identical to any one of SEQ ID NOs:9-11 or 15-20 (or ranges including and/or spanning the aforementioned values). In some embodiments, the composition comprises two or more (e.g., 2, 3, 4, 5, 6, etc.) RNAi(s). In some embodiments, the composition comprises one or more a small molecule inhibitors of PAX5. In some embodiments, the composition comprises a pharmaceutically acceptable carrier. In some embodiments, the one or more RNAi(s) comprises SEQ ID NO:15. In some embodiments, the one or more RNAi(s) comprises at least one of SEQ ID NO:15, SEQ ID NO:17, and SEQ ID NO:19. In some embodiments, one or more RNAi(s) comprises at least one of SEQ ID NO:16, SEQ ID NO:18, and SEQ ID NO:20. In some embodiments, the one or more RNAi(s) further comprises at least one of SEQ ID NO:9, SEQ ID NO:10, SEQ ID NO:11, SEQ ID NO:12, SEQ ID NO:13, and SEQ ID NO:14.

Some embodiments pertain to a method of reducing expression of a PAX5 gene in a cell. In some embodiments, the method comprises contacting a cell with the RNAi or RNAi(s), combinations of therapeutics, or a composition comprising the same. In some embodiments, the method comprises maintaining the cell for a time sufficient to obtain inhibition of the PAX5 gene, thereby reducing expression of the PAX5 gene in the cell. In some embodiments, the cell is isolated from (FIGS. 22A-22B) or is inside a subject (FIG. 22C). In some embodiments, the cell is contacted with the RNAi for a period of equal to or at least about: 8 hours, 16 hours, 48 hours, 72 hours, or ranges including and/or spanning the aforementioned values.

Some embodiments pertain to the cell made by a method as disclosed above or as disclosed elsewhere herein. In some embodiments, the target cell is non-senescent and/or has decreased senescent behavior, has increased innate immune function, increased telomere length, lower replicative stress relative to the patient cell, increased stem cell clonogenicity; increased cytotoxic function, increased mitogen- and/or antigen-induced lymphocyte proliferation and/or activation, decreased myeloid to lymphoid ratio, increased CD4 to CD8 T lymphocyte ratio, decreased expression of senescence-associated secretory proteins, and/or decreased expression of senescence- and/or aging-related genes.

Some embodiments pertain to a method of treating a subject having a disease or disorder that would benefit from reduction in expression of a PAX5 gene. In some embodiments, the method comprises administering to the subject a therapeutically effective amount of cells that have been treated with the RNAi, combination of RNAi(s), or compositions containing RNAi(s) as disclosed above or elsewhere herein, thereby treating the subject.

In some embodiments, the administration of the RNAi, combinations, or compositions disclosed herein result in a PAX5 expression that is reduced by at least about 30%, 40%, 50%, 60%, 70%, 80%, 90%, 95%, 98%, 99%, or 99.9%. In some embodiments, PAX5 expression is reduced by at least about 70%. In some embodiments, a reduction in the expression of PAX5 results in increased hematopoietic stem and progenitor cell clonogenicity, T cell activation and immune cell cytotoxicity and decreased expression of genes linked to cellular senescence and aging, as well as a decrease in the production of proteins composing the senescence-associated secretory phenotype (SASP). In some embodiments, PAX5 related diseases, such as age-related immune dysfunction are treated.

Some embodiments pertain to one or more RNAi(s) for reducing expression of a PPM1F gene. In some embodiments, as shown in FIGS. 22H and 22I, the method includes selecting or acquiring a patient cell 110. In some embodiments, as shown in FIG. 22C, the cell may be a cell in the body of the patient (for in vivo treatment) or isolated from the patient (for ex vivo and/or in vitro treatment). In some embodiments, as shown, the cell is treated by exposing it to one or more different PPM1F gene RNAi(s) 111′. In some embodiments, as shown, the RNAi(s) act within the cell for a period of time 112. In some embodiments, as shown, this results in a target cell 113. In some embodiments, as shown in FIG. 22I, the cell can be reintroduced to the patient (e.g., where it was initially isolated from the patient). In some embodiments, the entire process is performed in vivo and, therefore, the cell need not be reintroduced to the patient.

In some embodiments, the RNAi(s) for reducing expression of a PPM1F gene comprise any one or more of SEQ ID NO:12, SEQ ID NO:13, SEQ ID NO:14, SEQ ID NO:15, SEQ ID NO:16, SEQ ID NO:17, SEQ ID NO:18, SEQ ID NO:19, or SEQ ID NO:20. In some embodiments, the RNAi(s) for reducing expression of a PPM1F gene is at least about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, about 99.99%, or about 100% identical to any one of SEQ ID NOs:12-20 (or ranges including and/or spanning the aforementioned values). In some embodiments, the nucleic acid sequence is any one of the preceding identities to the denoted SEQ ID NOs: 12-20, and the nucleic acid include additional nucleotides, for example 1, 2, 3, 4, 5, 6, 7, 8, 9, 10, 15, 20, 25, 30 or more additional nucleotides, in a second section, which is contiguous to the first section. In some embodiments, one, two, or three amino acids of any one of SEQ ID NOs:12-20 can be exchanged with another amino acid (e.g., any one of G, A, S, T, C, V, L, I, M, P, F, Y, W, D, E, N, Q, H, K, R, etc.) to provide different RNAi, so long as the RNAi still hybridizes to its gene target to achieve binding and/or inhibition.

In some embodiments, an RNAi composition for reducing expression of a PPM1F gene is provided. In some embodiments, the composition comprises the one or more RNAi(s). In some embodiments, the composition comprises of any one or more of SEQ ID NO:12, SEQ ID NO:13, SEQ ID NO:14, SEQ ID NO:15, SEQ ID NO:16, SEQ ID NO:17, SEQ ID NO:18, SEQ ID NO:19, or SEQ ID NO:20. In some embodiments, the composition for reducing expression of a PPM1F gene comprises RNAi(s) that are at least about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, about 99.99%, or about 100% identical to any one of SEQ ID NOs:14-20 (or ranges including and/or spanning the aforementioned values). In some embodiments, the composition comprises two or more (e.g., 2, 3, 4, 5, 6, etc.) RNAi(s). In some embodiments, the composition comprises one or more a small molecule inhibitors of PPM1F. In some embodiments, the composition comprises a pharmaceutically acceptable carrier. In some embodiments, the one or more RNAi(s) comprises SEQ ID NO:15. In some embodiments, the one or more RNAi(s) comprises at least one of SEQ ID NO:15, SEQ ID NO:17, and SEQ ID NO:19. In some embodiments, one or more RNAi(s) comprises at least one of SEQ ID NO:16, SEQ ID NO:18, and SEQ ID NO:20. In some embodiments, the one or more RNAi(s) further comprises at least one of SEQ ID NO:9, SEQ ID NO:10, SEQ ID NO:11, SEQ ID NO:12, SEQ ID NO:13, and SEQ ID NO:14.

Some embodiments pertain to a method of reducing expression of a PPM1F gene in a cell. In some embodiments, the method comprises contacting a cell with the RNAi or RNAi(s), combinations of therapeutics, or a composition comprising the same. In some embodiments, the method comprises maintaining the cell for a time sufficient to obtain inhibition of the PPM1F gene, thereby reducing expression of the PPM1F gene in the cell. In some embodiments, the cell is isolated from (FIGS. 22A-B) or is inside a subject (FIG. 22C). In some embodiments, the cell is contacted with the RNAi for a period of equal to or at least about: 8 hours, 16 hours, 48 hours, 72 hours, or ranges including and/or spanning the aforementioned values.

Some embodiments pertain to the cell made by a method as disclosed above or as disclosed elsewhere herein. In some embodiments, the target cell is non-senescent and/or has decreased senescent behavior, has increased innate immune function, increased telomere length, lower replicative stress relative to the patient cell, increased stem cell clonogenicity; increased cytotoxic function, increased mitogen- and/or antigen-induced lymphocyte proliferation and/or activation, decreased myeloid to lymphoid ratio, increased CD4 to CD8 T lymphocyte ratio, decreased expression of senescence-associated secretory proteins, and/or decreased expression of senescence- and/or aging-related genes.

Some embodiments pertain to a method of treating a subject having a disease or disorder that would benefit from reduction in expression of a PPM1F gene. In some embodiments, the method comprises administering to the subject a therapeutically effective amount of cells that have been treated with the RNAi, combination of RNAi(s), or compositions containing RNAi(s) as disclosed above or elsewhere herein, thereby treating the subject.

In some embodiments, the administration of the RNAi, combinations, or compositions disclosed herein result in a PPM1F expression that is reduced by at least about 30%, 40%, 50%, 60%, 70%, 80%, 90%, 95%, 98%, 99%, or 99.9%. In some embodiments, PAX5 expression is reduced by at least about 70%. In some embodiments, a reduction in the expression of PPM1F results in increased hematopoietic stem and progenitor cell clonogenicity, T cell activation and immune cell cytotoxicity and decreased expression of genes linked to cellular senescence and aging, as well as a decrease in the production of proteins composing the senescence-associated secretory phenotype (SASP). In some embodiments, PPM1F related diseases, such as age-related immune dysfunction are treated.

In some embodiments, a reduction in the expression of CAMK2G results in increased hematopoietic stem and progenitor cell clonogenicity, T cell activation and immune cell cytotoxicity and decreased and decreased expression of genes linked to cellular senescence and aging, as well as a decrease in the production of proteins composing the senescence-associated secretory phenotype (SASP). In some embodiments, CAMK2G related diseases, such as age-related immune dysfunction are treated. In some embodiments, the expression of CAMK2G is reduced by equal to or at least about: 0.01%, 1%, 10%, 20%, 30%, 40%, 50%, 60%, 70%, 80%, 90%, 95%, 98%, 99%, 99.9%, 100%, or ranges including and/or spanning the aforementioned values.

In some embodiments, the method of reducing expression of the PAX5 gene and reducing expression of the PPM1F gene in a cell comprises contacting the cell with one or more interfering RNA(s) (RNAi(s)), wherein the one or more RNAi(s) include one or more of SEQ ID NOs:9-20. In some embodiments, the cell is maintained for a time sufficient to obtain inhibition of the PAX5 gene and the PPM1F gene, thereby reducing expression of the PAX5 gene and the PPM1F gene in that cell to provide a target cell.

In some embodiments, PPM1F and/or PAX5 expression is reduced by at least about: 0.01%, 1%, 10%, 20%, 30%, 40%, 50%, 60%, 70%, 80%, 90%, 95%, 98%, 99%, 99.9%, 100%, or ranges including and/or spanning the aforementioned values. In some embodiments, PPM1F and/or PAX5 expression is reduced by at least about 70%.

In some embodiments, the cell is contacted with the one or more RNAi(s) for a period of equal to or at least about: 8 hours, 16 hours, 48 hours, 72 hours, 84 hours, or ranges including and/or spanning the aforementioned values. In some embodiments, the cell is isolated from a subject. In some embodiments, the cell is inside a subject. In some embodiments, the cell is a human cell.

Some embodiments pertain to the target cell made by contacting the cell with one or more RNAi(s) that are at least about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, about 99.99%, or about 100% identical to one or more of SEQ ID NOs:9-20.

In some embodiments, the target cell is non-senescent and/or has decreased senescent behavior. In some embodiments, a cell that is non-senescent and/or has decreased senescent releases fewer cytokines and other secreted proteins that are related to the inflammatory response and/or are tumor-supportive, also referred to as the senescence-associated secretory phenotype (SASP), decreased levels of genes linked to senescence and aging, including but not limited to p53, p21 and p16^{INK4A}. In some embodiments, as a result, the patient has less soreness in joints, higher activity levels, less stiffness (e.g., in the legs, arms, and/or back), increased cognitive function, increased cardiovascular function, increased health span (e.g., which may include a decrease in the general incidence of age-related disease(s) over the course of chronological aging). In some embodiments, the target cell has decreased expression of senescence-associated secretory proteins, and/or decreased expression of senescence- and/or aging-related genes.

In some embodiments, the target cell has increased innate and adaptive immune function including increased T cell activation, and increased immune cell cytotoxicity. In some embodiments, as a result, the patient has less occurrences of sickness, periodically less episodes of general sickness including but not limited to the common cold, allergies, influenza, pneumonia, increased cancer immune surveillance resulting in clearance of pre-cancerous cells and lower incidence of cancer occurrence or relapse.

In some embodiments, the target cell has increased telomere length. Cells with a lower rate of aging-related telomere attrition display a lower rate of cellular senescence incidence. In some embodiments, as a result, the patient has less soreness in joints, higher activity levels, less stiffness (e.g., in the legs, arms, and/or back), increased cognitive function, increased cardiovascular function, increased health span (e.g., which may include a decrease in the general incidence of age-related disease(s) over the course of chronological aging).

In some embodiments, the target cell has lower replicative stress relative to the patient cell. In some embodiments, these cells display a lower rate of cellular senescence incidence. In some embodiments, as a result, the patient has less soreness in joints, higher activity levels, less stiffness (e.g., in the legs, arms, and/or back), increased cognitive function, increased cardiovascular function, increased health span (e.g., which may include a decrease in the general incidence of age-related disease(s) over the course of chronological aging).

In some embodiments, the target cell has increased stem cell clonogenicity. In some embodiments, this results in increased continuous production of a functional hematopoietics system comprised of immune cells exhibiting low levels of cellular senescence, increased immune activation and increased cytotoxic function. In some embodiments, these cells display a lower rate of cellular senescence incidence. In some embodiments, as a result, the patient has less soreness in joints, higher activity levels, less stiffness (e.g., in the legs, arms, and/or back), increased cognitive function, increased cardiovascular function, increased health span (e.g., which may include a decrease in the general incidence of age-related disease(s) over the course of chronological aging), less occurrences of sickness, periodically less episodes of general sickness including but not limited to the common cold, allergies, influenza, pneumonia, increased cancer immune surveillance resulting in clearance of pre-cancerous cells and/or lower incidence of cancer occurrence or relapse.

In some embodiments, the target cell has increased cytotoxic function. In some embodiments, increased cytotoxic function helps reduce potentially cancerous cells from replicating, enhances the detection and clearance of pre-cancerous and cancerous cells by the innate and adaptive immune systems, and/or increases systemic immune surveillance to prevent the formation of circulating tumor cells.

In some embodiments, the target cell has increased mitogen- and/or antigen-induced lymphocyte proliferation and/or activation. In some embodiments, this results in the patient having less occurrences of sickness, periodically less episodes of general sickness including but not limited to the common cold, allergies, influenza, pneumonia, increased cancer immune surveillance resulting in clearance of pre-cancerous cells, and/or lower incidence of cancer occurrence or relapse.

In some embodiments, the target cell has decreased myeloid to lymphoid ratio. In some embodiments, this results in the patient having less occurrences of sickness, periodically less episodes of general sickness including but not limited to the common cold, allergies, influenza, pneumonia, increased cancer immune surveillance resulting in clearance of pre-cancerous cells, and/or lower incidence of cancer occurrence or relapse. In some embodiments, these cells display a lower rate of cellular senescence incidence. In some embodiments, as a result, the patient has less soreness in joints, higher activity levels, less stiffness (e.g., in the legs, arms, and/or back), increased cognitive function, increased cardiovascular function, increased health span (e.g., which may include a decrease in the general incidence of age-related disease(s) over the course of chronological aging), less occurrences of sickness, periodically less episodes of general sickness including but not limited to the common cold, allergies, influenza, pneumonia, increased cancer immune surveillance resulting in clearance of pre-cancerous cells, and/or lower incidence of cancer occurrence or relapse. In some embodiments, a decreased myeloid to lymphoid ratio helps reduce potentially cancerous cells from replicating, enhances the detection and clearance of pre-cancerous and cancerous cells by the innate and adaptive immune systems, and/or increases systemic immune surveillance to prevent the formation of circulating tumor cells.

In some embodiments, the target cell has increased CD4 to CD8 T lymphocyte ratio. In some embodiments, this results in the patient having less occurrences of sickness, periodically less episodes of general sickness including but not limited to the common cold, allergies, influenza, pneumonia, increased cancer immune surveillance resulting in clearance of pre-cancerous cells and lower incidence of cancer occurrence or relapse. In some embodiments, these cells display a lower rate of cellular senescence incidence. In some embodiments, as a result, the patient has less soreness in joints, higher activity levels, less stiffness (e.g., in the legs, arms, and/or back), increased cognitive function, increased cardiovascular function, increased health span (e.g., which may include a decrease in the general incidence of age-related disease(s) over the course of chronological aging), less occurrences of sickness, periodically less episodes of general sickness including but not limited to the common cold, allergies, influenza, pneumonia, increased cancer immune surveillance resulting in clearance of pre-cancerous cells, and/or lower incidence of cancer occurrence or relapse. In some embodiments, an increased CD4 to CD8 T lymphocyte ratio helps reduce potentially cancerous cells from replicating, enhances the detection and clearance of pre-cancerous and cancerous cells by the innate and adaptive immune systems and/or increases systemic immune surveillance to prevent the formation of circulating tumor cells.

In some embodiments, a decrease in senescence is measured as a decrease scenescense indicators, such as any senescence cytokine or protein, a senescence related inflammatory cytokine, a senescence related tumor supportive cytokine, and/or as a decrease in the amount of senescnence related gene expression (e.g., p53, p21, p16INK4A, and/or any ligand disclosed in FIG. 20). In some embodiments, patients receiving treatment experience a decrease in the senescence indicators of equal to or at least about: 5%, 10%, 25%, 50%, 75%, 90%, or ranges including and/or spanning the aforementioned values. In some embodiments, soreness and/or stiffness is quantified by measuring pain during the movement of a joint (e.g., extending the leg at the knee or the arm at the elbow) using one or more of the following pain scales: Visual Analog Scale for Pain (VAS Pain), Numeric Rating Scale for Pain (NRS Pain), McGill Pain Questionnaire (MPQ), Short-Form McGill Pain Questionnaire (SF-MPQ), Chronic Pain Grade Scale (CPGS), Short Form-36 Bodily Pain Scale (SF-36 BPS), and Measure of Intermittent and Constant Osteoarthritis Pain (ICOAP). In some embodiments, in patients receiving treatment, pain, soreness, and/or stiffness decreases by equal to or at least about: 5%, 10%, 25%, 50%, 75%, 90%, or ranges including and/or spanning the aforementioned values. In some embodiments, cognitive function is quantified using one or more of the following cognition scales: Cognitive Function Composite Score, Fluid Cognition Composite Score (includes DCCS, Flanker, Picture Sequence Memory, Mini-Mental State Exam (MMSE), etc.). In some embodiments, cognition is improved in patients receiving treatment by equal to or at least about: 5%, 10%, 25%, 50%, 75%, 90%, or ranges including and/or spanning the aforementioned values. In some embodiments, cardiac function is measured using cardiac output (e.g., the measure of a heart's ability to pump). In some embodiments, cardiac output is improved in patients receiving treatment by equal to or at least about: 5%, 10%, 25%, 50%, 75%, 90%, or ranges including and/or spanning the aforementioned values. In some embodiments, life span is increased in patients receiving treatment by equal to or at least about: 5%, 10%, 15%, 25%, or ranges including and/or spanning the aforementioned values. In some embodiments, rates of sickness (e.g., occurrences of common cold, allergies, influenza, pneumonia, etc.) in patients receiving treatment decreases by equal to or at least about: 5%, 10%, 25%, 50%, 75%, 90%, or ranges including and/or spanning the aforementioned values. In some embodiments, telomere length in patients receiving treatment is higher by equal to or at least about: 5%, 10%, 25%, 50%, 75%, 90%, or ranges including and/or spanning the aforementioned values. In some embodiments, cytotoxic function in patients receiving treatment is higher by equal to or at least about: 5%, 10%, 25%, 50%, 75%, 90%, or ranges including and/or spanning the aforementioned values. In some embodiments, increased cancer cell surveillance is measured by an increase in the clearance of cancer cells from patients receiving treatment by equal to or at least about: 5%, 10%, 25%, 50%, 75%, 90%, or ranges including and/or spanning the aforementioned values. In some embodiments, cancer rates in patients receiving treatment is lower by equal to or at least about: 25%, 50%, 75%, 90%, or ranges including and/or spanning the aforementioned values. In some embodiments, the health of a treated patient is compared to the health of an untreated, control patient to determine any effect of the treatment. In some embodiments, the health of a treated patient is compared to the health of a that patient before treatment to determine any effect of the treatment.

Some embodiments pertain to a composition for reducing expression of a PAX5 gene and reducing the expression of a PPM1F gene (and/or the other genes encoding the other proteins disclosed herein), the composition comprising an acceptable carrier and an RNAi that is at least about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, about 99.99%, or about 100% identical to one or more of SEQ ID NOs:9-20. In some embodiments, the composition comprises an acceptable carrier and an RNAi that is at least 80% to 100% identical to SEQ ID NO:15. In some embodiments, the composition comprises or further comprises a microRNA that is at least 80% to 100% identical to SEQ ID NO:16. In some embodiments, the composition comprises or further comprises a microRNA that is at least 80% to 100% identical to SEQ ID NO:17. In some embodiments, the composition comprises or further comprises a microRNA that is at least 80% to 100% identical to SEQ ID NO:18. In some embodiments, the composition comprises or further comprises a microRNA that is at least 80% to 100% identical to SEQ ID NO:19. In some embodiments, the composition comprises or further comprises a microRNA that is at least 80% to 100% identical to SEQ ID NO:20. In some embodiments, the composition comprises or further comprises one or more of SEQ ID NOs:9-14.

In some embodiments, the RNAi or combination of RNAi(s) (e.g., siRNAs, microRNAs, etc.) are administered using a pharmaceutically acceptable carrier. In some embodiments the wherein the pharmaceutically acceptable carrier comprises one or more of nanoparticles composed of non-degradable and/or degradable biomaterials, micelles, liposomes, extracellular vesicles (native and/or synthetic), exosomes (native and/or synthetic), and/or microvesicles (native and/or synthetic). In some embodiments, the pharmaceutically acceptable carrier comprises a non-natural or synthetic exosome. In some embodiments, natural exosomes are not used as a carrier. In some embodiments, RNAi can be delivered to the targets cells for active transport-mediated uptake, by electroporation and/or by nucleofection.

In some embodiments, the RNAi or combination of RNAi(s) are administered using gene delivery vector. In some embodiments, the carrier is a viral vector. In some embodiments, a gene for the transcription of the RNAi is delivered to a cell which transcribes the RNAi. In some embodiments, the RNAi or combination of RNAi(s) are administered to other cells by a cell that transcribes the RNAi or RNAi(s) (e.g., through exosome, extracellular vesicles, etc.). In some embodiments, the cell transcribing the RNAi is treated by the RNAi it produces.

In some embodiments, a dose of RNAi(s) comprises equal to less than about: 0.001 μg, 0.01 μg, 0.1 μg, 1 μg, 10 μg, 100 μg, 1000 μg, or ranges including and/or spanning the aforementioned values.

Some embodiments pertain to a method of treating a subject having a disease or disorder that would benefit from reduction in expression of a PAX5 gene and a reduction in expression of a PPM1F gene. As shown in FIG. 22J, in some embodiments, the method includes acquiring a patient cell 110. In some embodiments, as shown in FIG. 22C, the cell may be a cell in the body of the patient (for in vivo treatment) or isolated from the patient (for ex vivo and/or in vitro treatment), as shown in FIG. 22A-B. In some embodiments, as shown, the cell is treated by exposing it to one or more different PAX5 gene RNAi(s) 111′ and/or one or more different PPM1F gene RNAi(s) 111′. In some embodiments, as shown, the RNAi(s) are allowed to act on the cell for a period of time 112. In some embodiments, as shown, this results in a target cell 113. In some embodiments, a patient suffering from a disease or disorder that would benefit from reduction of the expression of a PAX5 gene and a reduction in expression of a PPM1F gene is selected 115. In some embodiments, as shown in FIG. 22J, the cell can be administered to the patient (e.g., where it was initially isolated from the patient). In some embodiments, the method comprises administering to the subject a therapeutically effective amount of the target cell as disclosed elsewhere herein or the composition as disclosed elsewhere herein, thereby treating the subject.

Some embodiments pertain to a method for treating or preventing a disease state, comprising administering to a patient in need thereof a therapeutically effective dose of cells treated with one or more RNAi(s) of a PAX5 gene and/or of a PPM1F gene. In some embodiments, the one or more RNAi(s) is selected from any one or more of the RNAis as recited in elsewhere herein. In some embodiments, the disease state is an age related dysfunction. In some embodiments, the disease state is not an age-related dysfunction. In some embodiments, the disease state comprises one or more of arthritis, atherosclerosis, breast cancer, cardiovascular disease, cataracts, chronic obstructive pulmonary disease, colorectal cancer, hypertension, osteoporosis, periodontitis, type 2 diabetes, immune dysfunction, Alzheimer's disease, leukemia, lymphoma, multiple sclerosis, Crohn's disease, HIV, influenza, pneumonia, lung cancer, melanoma, stroke, Parkinson's disease, and multiple drug resistant Staphylococcus aureus (MRSA). In some embodiments, the dysfunction is one in which PAX5 and/or PPM1F plays a direct role or is part of a pathway that is compromised. In some embodiments, PAX5 and/or PPM1F are not part of a compromised pathway, but allow for the recovery or increase in activity to address the disorder.

Some embodiments pertain to a method for preparing a target cell. In some embodiments, the method comprises obtaining cells from a subject to provide at least one subject cell. In some embodiments, the method comprises exposing the at least one subject cell to one or more RNAi(s) that is at least about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, about 99.99%, or about 100% identical to one or more of SEQ ID NOs:9-20 to provide at least one target cell. In some embodiments, the at least one target cell is member of a population of cells comprising equal to or at least about 100, 1000, 10,000, 100,000, 1,000,000, or 10,000,000 cells. Some embodiments pertain to the target cell.

Some embodiments pertain to a method for treating or preventing cellular dysfunction in a patient. In some embodiments, the method comprises administering to a patient in need thereof a therapeutically effective dose of the target cell as disclosed above or elsewhere herein. In some embodiments, the method comprises administering to a patient in need thereof a therapeutically effective dose of one or more RNAi(s) and small molecule inhibitors. In some embodiments, the cellular dysfunction is an age-related dysfunction. In some embodiments, the cellular dysfunction is not an age-related dysfunction.

Some embodiments pertain to an interfering RNA (RNAi) for reducing the expression of a paired any one of the CAMK2G/CAMK-II, PAK, C21orf62-AS1, CASP14, CATSPER2, DNAH10OS, ELMOD1, GALNT6, HEPN1, LANCL2, LL22NC03-63E9.3, PPTC7, PROSC, RAB3B, RRP7A, SERF1A/SERF1B, SLC35E3, SMIM10, SPRY3, SUMO2, TPP1, TPPP, WBP1L, ZNF33A, or ZNF549 gene. In some embodiments, the RNAi comprises 4 to 50 contiguous nucleotides having a polynucleotide sequence that is at least about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, about 99.99%, or about 100% identical complementary (or anti-complementary) to a region of any one of the CAMK2G/CAMK-II, PAK, C21orf62-AS1, CASP14, CATSPER2, DNAH10OS, ELMOD1, GALNT6, HEPN1, LANCL2, LL22NC03-63E9.3, PPTC7, PROSC, RAB3B, RRP7A, SERFiA/SERFiB, SLC35E3, SMIM10, SPRY3, SUMO2, TPP1, TPPP, WBP1L, ZNF33A, or ZNF549 gene. In some embodiments, the RNAi comprises 4 to 50 contiguous nucleotides having a polynucleotide sequence that is at least about 60%, about 70%, about 80%, about 85%, about 86%, about 87%, about 88%, about 89%, about 90%, about 91%, about 92%, about 93%, about 94%, about 95%, about 96%, about 97%, about 98%, or about 100% identical to a region of any one or more of SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20.

Some embodiments pertain to a method for reducing the expression of a PAX5 gene and/or PPM1F gene, comprising exposing a cell at least one isolated microRNA that is at least about 60%, about 70%, about 80%, about 85%, about 86%, about 87%, about 88%, about 89%, about 90%, about 91%, about 92%, about 93%, about 94%, about 95%, about 96%, about 97%, about 98%, or about 100% identical to any one or SEQ ID NO:15-20. Some embodiments pertain to a method for reducing the expression of a PAX5 and/or PPM1F gene, comprising exposing a cell to at least one isolated microRNA, wherein the at least one microRNA is at least about 60%, about 70%, about 80%, about 85%, about 86%, about 87%, about 88%, about 89%, about 90%, about 91%, about 92%, about 93%, about 94%, about 95%, about 96%, about 97%, about 98%, or about 100% identical to SEQ ID NO:15. Some embodiments pertain to a method for reducing the expression of a PAX5 gene and/or PPM1F, comprising exposing a cell to a composition comprising at least one isolated microRNA, wherein the at least one microRNA is at least about 60%, about 70%, about 80%, about 85%, about 86%, about 87%, about 88%, about 89%, about 90%, about 91%, about 92%, about 93%, about 94%, about 95%, about 96%, about 97%, about 98%, or about 100% identical to SEQ ID NO:16. In some embodiments, the method comprises administering both SEQ ID NO: 15 and SEQ ID NO: 16. In some embodiments, the method comprises administering a microRNA that is at least about 60%, about 70%, about 80%, about 85%, about 86%, about 87%, about 88%, about 89%, about 90%, about 91%, about 92%, about 93%, about 94%, about 95%, about 96%, about 97%, about 98%, or about 100% identical to SEQ ID NO:17. In some embodiments, the method comprises administering a microRNA that is at least about 60%, about 70%, about 80%, about 85%, about 86%, about 87%, about 88%, about 89%, about 90%, about 91%, about 92%, about 93%, about 94%, about 95%, about 96%, about 97%, about 98%, or about 100% identical to SEQ ID NO:18. In some embodiments, the method comprises administering a microRNA that is at least about 60%, about 70%, about 80%, about 85%, about 86%, about 87%, about 88%, about 89%, about 90%, about 91%, about 92%, about 93%, about 94%, about 95%, about 96%, about 97%, about 98%, or about 100% identical to SEQ ID NO:19. In some embodiments, the method comprises administering a microRNA that is at least about 60%, about 70%, about 80%, about 85%, about 86%, about 87%, about 88%, about 89%, about 90%, about 91%, about 92%, about 93%, about 94%, about 95%, about 96%, about 97%, about 98%, or about 100% identical to SEQ ID NO:20.

In some embodiments, a nucleic acid is an oligonucleotide, an antisense oligonucleotide, an RNAi agent, a miRNA, immunomodulatory nucleic acid, an aptamer, a Piwi-interacting RNA (piRNA), a small nucleolar RNA (snoRNA), a ribozyme, a mRNA, a lncRNA, a ncRNA, an antigomir (e.g., an antagonist to a miRNA, IncRNA, ncRNA or other nucleic acid), or a portion thereof. In some embodiments, an interfering polynucleotide is provided. In some embodiments, the nucleic acids are single stranded oligonucleotides. In some embodiments, the nucleic acids are double stranded oligonucleotides. In some embodiments, the sequence of an antisense RNAi is complementary to the protein-coding sequence. The nucleic acids described herein may be any of a range of length of up to, but not necessarily 200 nucleotides in the case of antisense oligonucleotides, RNAi, siRNA, shRNA, iRNA, antagomirs. In some embodiments, the antisense RNA is modified, for example by incorporating non-naturally occurring nucleotides. In some embodiments, the nucleic acid is an interfering RNA, such as an siRNA, that specifically targets an RNA molecule, such as an mRNA, encoding a protein involved in a disease, such as cancer. In some embodiments, the disease is cancer, such as a solid tumor or hematological malignancy, and the interfering RNA targets mRNA encoding a protein involved in the cancer, such as a protein involved in regulating the progression of the cancer. In some embodiments, the nucleic acid is an interfering RNA, such as an siRNA, that specifically targets an RNA molecule, such as an mRNA, encoding a protein involved in negatively regulating an immune response. In some embodiments, the interfering RNA targets mRNA encoding a negative co-stimulatory molecule.

In some embodiments, the nucleic acids are miRNA. A microRNA (abbreviated miRNA) is a short ribonucleic acid (RNA) molecule found in eukaryotic cells. A microRNA molecule has very few nucleotides (an average of 22) compared with other RAs. miRNAs are post-transcriptional regulators that bind to complementary sequences on target messenger RNA transcripts (mRNAs), usually resulting in translational repression or target degradation and gene silencing. In some embodiments, the miRNAs substantially complementary (such as at least about 60%, 70%, 80%, 90%, 95%, 98%, 99%, 99.9%, or more identical to) the corresponding target gene. In some embodiments, the antisense RNA is modified, for example by incorporating non-naturally occurring nucleotides.

Some embodiments disclosed herein relate to compositions and methods for decreasing the extent of cellular senescence occurring in the lymphohematopoietic system of a subject. In some embodiments, a method of reducing the extent of cellular senescence occurring in the lymphohematopoietic system of a subject comprises administration of a composition comprising a microRNA (miR) that targets and decreases the expression of PAX5, PPM1F, or both in a subject. Aspects of the present disclosure are directed to compositions and methods for performing RNA-induced gene silencing (also called RNAi) of PAX5, PPM1F or both.

The compositions and methods disclosed herein may provide means of restoring an aging lymphohematopoietic system. Herein reference is made to the undifferentiated cells of the hematopoietic lineage including hematopoietic stem cells (HSCs), lymphoid progenitor cells (LPCs) and myeloid progenitor cells (MPCs) which are known collectively as lymphohaematopoietic progenitor cells (LPCs). LPCs and MPCs are each formed by the differentiation of HSCs.

Other examples of differentiated cells of the hematopoietic lineage include T lymphocytes, B lymphocytes, eosinophils, basophils, neutrophils, megakaryocytes, monocytes, macrophages erythrocytes, granulocytes, mast cells, dendritic cells and natural killer cells. The pathways of differentiation in the lymphohematopoietic system have been extensively characterized and the various cell stages are readily identifiable according to morphology and lineage-specific cell surface markers.

In some embodiments, the compositions and methodologies disclosed herein are utilized in the treatment of one or more ARDs that may be the result of an aging lymphohematopoietic system. In certain aspects, the present disclosure contemplates a polynucleotide comprising a unimolecular RNA, such as an shRNA. The shRNA can be a unimolecular RNA that includes a sense sequence, a loop region, and an antisense sequence (sometimes referred to as first and second regions) which together form a hairpin loop structure. A loop structure can also include deoxyribonucleotides, non-nucleotide monomers and reversible linkages such as S—S bonds, which can be formed by oxidation of —SH groups introduced into nucleotide residues.

The antisense and sense sequences of the RNAi may be substantially complementary to one other (about 80% complementary or more), where in certain aspects the antisense and sense sequences are 100% complementary to each other. In certain aspects, the antisense and sense sequences are too short to be processed by Dicer, and hence act through an alternative pathway to that of longer double-stranded RNAs. Additionally, the antisense and sense sequences within a unimolecular RNA of the present disclosure can be the same length, or differ in length by from about 1 base to about 5 bases. The loop can be any length, such as from 0 to 4 nucleotides (nt) in length or an equivalent length of non-nucleotidic linker, and or 2 nucleotides or an equivalent length of non-nucleotidic linker (e.g., a non-nucleotide loop having a length equivalent to 2 nt). In the case of a loop of zero nt, the antisense sequence is linked directly to the sense sequence, with part of one or both strands forming the loop. In another aspect of a zero-nt loop shRNA, the antisense sequence is about 18 or 19 nt and the sense sequence is shorter than the antisense sequence, so that one end of the antisense sequence forms the loop.

In one aspect, an shRNA described herein comprises a sequence complementary to a sequence of an mRNA of PAX5. PAX5 encodes the B-cell lineage specific activator protein (BSAP) which is a nuclear protein in the paired-box containing (PAX) family of transcription factors involved in control of organ development and tissue differentiation. BSAP is primarily expressed in B lymphocytes and B-cell lymphomas, with additional expression in the developing central nervous system. In an aspect of the present disclosure a method of treating an ARD comprises administering to a subject an RNAi that reduces the expression of BSAP. In some embodiments, the RNAi is an shRNA which acts to reduce the expression of BSAP by mechanisms such as mRNA disruption (e.g., hydrolysis, slicing) or translational repression.

In some embodiments, an RNAi for reducing the expression of BSAP comprises an shRNA having SEQ ID NOs: 9-11, or combinations thereof.

In some embodiments, the RNAi (e.g., shRNA) is capable of binding to a target sequence of PAX5 mRNA and reducing the expression of BSAP by from about 30% to about 100%, alternatively from about 30% to about 50%, alternatively from about 50% to about 75% or alternatively from about 75% to about 100% of the original expression level of BSAP. The expression level of BSAP in any particular cell type may be determined using any suitable methodology for determining protein expression level such as Western blots, high performance liquid chromatography, enzyme-linked immunosorbent assay, protein immunoprecipitation and the like.

In one aspect, an shRNA described herein comprises a sequence complementary to a sequence of an mRNA of PPM1F. PPM1F encodes Mg2+/Mn2+-dependent protein phosphatase 1F which belongs to the PP2C family of Ser/Thr protein phosphatases. PP2C family members are known as negative regulators of cell stress response pathways, including p38 MAPK, JNK and HOG signaling pathways. Known substrates of the phosphatase include Rho guanine nucleotide exchange factors (PIX) and calcium/calmodulin-dependent protein kinase II gamma. PPM1F is ubiquitous in various tissues and organs.

In some embodiments, an RNAi for downregulation of the expression of the gene product of PPM1F comprises an shRNA having SEQ ID Nos:12-14 or combinations thereof. In some embodiments, the RNAi (e.g., shRNA) is capable of binding to a target sequence of the PPM1F mRNA and reducing the expression of the Mg2+/Mn2+-dependent protein phosphatase 1F by from about 30% to about 100%, alternatively from about 30% to about 50%, alternatively from about 50% to about 75% or alternatively from about 75% to about 100% of the original expression level of Mg2+/Mn2+-dependent protein phosphatase 1F. The expression level of Mg2+/Mn2+-dependent protein phosphatase 1F in any particular cell type may be determined using any suitable methodology for determining protein expression level such as Western blots, high performance liquid chromatography, enzyme-linked immunosorbent assay, protein immunoprecipitation and the like.

In some aspects of the present disclosure, an RNAi (e.g., shRNA) that reduces the expression of the mRNA of PAX5, PPM1F or both comprises a modified nucleotide. One or more of the nucleotides present in the RNAi (e.g., shRNA) may be modified to achieve one or more user and/or process goals, such as increased stability. Modified bases refer to nucleotide bases such as, for example, adenine, guanine, cytosine, thymine, uracil, xanthine, inosine, and queuosine that have been modified by the replacement or addition of one or more atoms or groups. Some examples of types of modifications that can comprise nucleotides that are modified with respect to the base moieties include but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles. The term nucleotide is also meant to include what are known in the art as universal bases. By way of example, universal bases include but are not limited to 3-nitropyrrole, 5-nitromdole, or nebularine. The term “nucleotide” is also meant to include the N3′ to P5′ phosphoramidate, resulting from the substitution of a ribosyl 3′ oxygen with an amine group. Further, the term nucleotide also includes those species that have a detectable label, such as for example a radioactive or fluorescent moiety, or mass label attached to the nucleotide.

In some aspects of the present disclosure, an RNAi (e.g., shRNA) that reduces the expression of the mRNA of PAX5, PPM1F or both is a component of an antibody conjugate. In some embodiments, any RNAi suitable for use in the reduction of the PAX5 or PPM1F may be utilized in the present disclosure. Additionally any suitable method of introducing such biomolecules to cells and/or administering such biomolecules is also contemplated. In some embodiments, the RNAi (e.g., shRNA) may be administered as a component of an antibody-RNAi (e.g., shRNA) conjugate. In some aspects, the antibody-shRNA conjugates specifically target senescent cells to deliver an shRNA molecule that reduces the expression of PAX5, PPM1F or both. The antibody-shRNA conjugates (or “complexes”) include an antibody or functional fragment thereof that targets a cell to selectively deliver an associated shRNA molecule to the cell. In some embodiments, the cell is a cell of the lymphohematopoietic system,

An antibody or functional antibody fragment is a molecule that includes one or more portions of an immunoglobulin or immunoglobulin-related molecule that specifically binds to, or is immunologically reactive with an age-related antigen or other age-related biomarker. The antibody may be a polyclonal antibody, a monoclonal antibody, or any suitable modified antibody. The term modified antibody includes, but is not limited to genetically engineered or otherwise modified forms of immunoglobulins, such as intrabodies, chimeric antibodies, fully human antibodies, humanized antibodies, and heteroconjugate antibodies (e.g., bispecific antibodies, diabodies, triabodies, and tetrabodies). The term functional antibody fragment includes one or more antigen binding fragments of an antibody alone or in combination with other molecules. The antibody-shRNA conjugates or complexes may be synthesized or constructed using any suitable conjugation method. In one aspect, the antibody-siRNA complex is constructed by a method of covalent conjugation. Synthesis of antibody-shRNA conjugates via a covalent construction strategy involves chemically linking an shRNA molecule to an antibody using a cleavable or non-cleavable.

In some aspects of the present disclosure, an RNAi (e.g., shRNA) that reduces the expression of the mRNA of PAX5, PPM1F or both is a component of a nucleic acid vector, or is encoded by a nucleic acid vector. A nucleic acid vector is any nucleic acid that functions to carry, harbor or express a nucleic acid of interest. Nucleic acid vectors can have specialized functions such as expression, packaging, pseudotyping, transduction or sequencing, for example. Nucleic acid vectors also can have, for example, manipulatory functions such as a cloning or shuttle vector. The structure of the vector can include any desired form that is feasible to make and desirable for a particular use. In an alternative aspect, a method of the present disclosure comprises introducing to the cells a vector capable of inducible or constitutive expression of one or more nucleotides of the type disclosed herein. For example, an expressible form of the shRNA may be located on a vector such as a plasmid, cosmid, phagemid, virus, and other vehicles derived from viral or bacterial sources.

In some embodiments, the RNAi (e.g., shRNA) that reduces the expression of the mRNA of PAX5, PPM1F or both is a component of a packaging construct. A packaging construct is a nucleic acid vector that encodes retroviral structural polypeptides sufficient for vector production. In some embodiments, the nucleotide vector is a viral vector comprises an oligonucleotide (e.g., siRNA, shRNA) that inhibits expression of the mRNA or gene product of PAX5, PPM1F or both. The viral vector may be a lentivirus vector, including an integrating lentivirus vector. A lentivirus is an icosahedral enveloped virus having a diploid RNA genome that becomes integrated into the host chromosome as a proviral DNA for genome replication. The lentiviral genome contains gag, pol and env genes which encode the structural polypeptides of the virion (p17, p24, p7 and p6); the viral enzymes protease, reverse transcriptase and integrase, and the envelope glycoproteins (gp120 and gp41), respectively.

In some aspects, the RNAi and/or SAD may be associated with a delivery material that facilitates entry of the SAD into the appropriate cells. In some embodiments, a SAD (e.g., an RNAi) of the type disclosed herein is associated with a liposome or niosome. In some embodiments, a SAD (e.g., an RNAi) is delivered using a viral vector. Liposomes are a form of vesicles that consist either of many, few or just one phospholipid bilayers. The polar character of the liposomal core enables polar drug molecules to be encapsulated. Amphiphilic and lipophilic molecules are solubilized within the phospholipid bilayer according to their affinity towards the phospholipids. Participation of nonionic surfactants instead of phospholipids in the bilayer formation results in niosomes. shRNA of the type disclosed herein (i.e., SADs) can be incorporated without loss of their activity within the hydrophobic domain of vesicle membranes, acting as a size-selective filter, only allowing passive diffusion of small solutes such as ions, nutrients and antibiotics. Thus, SADs may be encapsulated in a nanocage and are effectively protected from premature degradation by proteolytic enzymes.

In an aspect the SAD is associated with a dendrimer. Dendrimers are nanometer-sized, highly branched and monodisperse macromolecules with symmetrical architecture. They consist of a central core, branching units and terminal functional groups. The core together with the internal units, determine the environment of the nanocavities and consequently their solubilizing properties, whereas the external groups the solubility and chemical behavior of these polymers. Targeting effectiveness is affected by attaching targeting ligands at the external surface of dendrimers, while their stability and protection from the Mononuclear Phagocyte System (MPS) is being achieved by functionalization of the dendrimers with polyethylene glycol chains (PEG)

In some embodiments, the SAD is associated with a liquid crystal. Liquid crystals combine the properties of both liquid and solid states. They can be made to form different geometries, with alternative polar and non-polar layers (i.e., a lamellar phase) where aqueous drug solutions can be included.

In some embodiments, the SAD is associated with a nanoparticle. Nanoparticles (including nanospheres and nanocapsules of size 10-200 nm) are in the solid state and are either amorphous or crystalline. They are able to adsorb and/or encapsulate a drug, thus protecting it against chemical and enzymatic degradation. Nanocapsules are vesicular systems in which the drug is confined to a cavity surrounded by a unique polymer membrane, while nanospheres are matrix systems in which the drug is physically and uniformly dispersed. Nanoparticles as drug carriers can be formed from both biodegradable polymers and non-biodegradable polymers. In recent years, biodegradable polymeric nanoparticles have attracted considerable attention as potential drug delivery devices in view of their applications in the controlled release of drugs, in targeting particular organs/tissues, as carriers of DNA in gene therapy, and in their ability to deliver proteins, peptides and genes through the peroral route.

In some embodiments, the SAD is associated with a hydrogel. Hydrogels are three-dimensional, hydrophilic, polymeric networks capable of imbibing large amounts of water or biological fluids. The networks are composed of homopolymers or copolymers, and are insoluble due to the presence of chemical crosslinks (tie-points, junctions), or physical crosslinks, such as entanglements or crystallites. Hydrogels exhibit a thermodynamic compatibility with water, which allows them to swell in aqueous media. They are used to regulate drug release in reservoir-based, controlled release systems or as carriers in swellable and swelling-controlled release devices. On the forefront of controlled drug delivery, hydrogels as enviro-intelligent and stimuli-sensitive gel systems modulate release in response to pH, temperature, ionic strength, electric field, or specific analyte concentration differences. In these systems, release can be designed to occur within specific areas of the body (e.g., within a certain pH of the digestive tract) or also via specific sites (adhesive or cell-receptor specific gels via tethered chains from the hydrogel surface). Hydrogels as drug delivery systems can be very promising materials if combined with the technique of molecular imprinting.

SADS associated with one or more of the delivery systems disclosed herein may be considered as packaged therapeutic agents and are herein denoted “p-SADs.” p-SADs may be further modified to improve properties such as bioavailability by modification of the packaging using any suitable methodology (e.g., conjugation with a targeting molecule).

In some embodiments, the present disclosure contemplates the utilization of SADs of the type disclosed herein as compositions for administration to a subject in need thereof. In some embodiments, the SADs may be a component of a pharmaceutical formulation that is administered locally or systemically to a subject. In some embodiments, SADs of the type disclosed herein are used in conjunction with a vehicle such as a nanoparticle, micelle, liposome, niosome, microsphere, cyclodextrin and the like. In some embodiments, such vehicles further comprise one or more elements to direct the carrier or vehicle to a particular cell, tissue or organ of a subject (e.g., cells of the lymphohematopoietic system).

In an aspect of the present disclosure, a subject experiencing an ARD may be administered a SAD of the type disclosed herein. The administration may involve targeting cells of the lymphohematopoietic system such using an adoptive therapy method comprising (i) obtaining lymphohematopoietic cells from the subject using any suitable methodology such as via mobilization of the stem cells into the peripheral blood, aspiration of the bone marrow, apheresis; (ii) transfecting or transducing the obtained cells with RNAi of the type disclosed herein and (iii) reintroducing the cells to the subject. An alternative methodology may comprise packaging an RNAi of the type disclosed herein such as via encapsulation and introducing the packaged RNAi to the bone marrow of a subject experiencing an ARD using any suitable administration route. The administration may involve targeting cells of the lymphohematopoietic system such using an adoptive therapy method comprising (i) obtaining lymphohematopoietic cells from the subject using any suitable methodology such as via mobilization of the stem cells into the peripheral blood, aspiration of the bone marrow, apheresis; (ii) transfecting or transducing the obtained cells with RNAi of the type disclosed herein using transposons such as sleeping beauty, crisper, CAS9, and zinc finger nucleases and (iii) reintroducing the cells to the subject. In such aspects, the expression of natural or synthetic nucleic acids encoding SADs of the type disclosed herein is typically achieved by operably linking a nucleic acid encoding the SAD polypeptide or portions thereof to a promoter, and incorporating the construct into an expression vector. The vectors can be suitable for replication and integration in eukaryotes. Typical cloning vectors contain transcription and translation terminators, initiation sequences, and promoters useful for regulation of the expression of the desired nucleic acid sequence.

In some embodiments, one or more of CRISPR, antibodies, and/or RISC can be used to reduce the expression of one or more of the proteins disclosed herein.

### Small Molecule Inhibitors

Disclosed herein is a method of decreasing the extent of cellular senescence occurring in the lymphohematopoietic system of a subject and/or treating an age-related disorder (ARD) that comprises administering to a subject in need thereof an effective amount of one or more of a RNAi, a chemical compound, and/or a target cell that has been treated with one of the foregoing. Some embodiments disclosed herein pertain to compositions and methods for decreasing the extent of cellular senescence occurring in the lymphohematopoietic system of a subject. In some embodiments, a method of decreasing the extent of cellular senescence occurring in the lymphohematopoietic system of a subject comprises administration of a composition comprising a polycyclic aromatic small molecule that targets and reduces the activity of the B-cell lineage specific activator protein, the protein phosphatase 1F enzyme or both in a subject. In some embodiments, the compositions and methodologies disclosed herein are utilized in the treatment of one or more age-related disorders.

Disclosed herein is a composition or compound comprising a polycyclic aromatic small molecule capable of targeting BSAP and reducing the activity of BSAP. Also disclosed herein is a composition comprising a polycyclic aromatic small molecule capable of targeting PP1F and reducing the activity of PP1F. In some embodiments, as shown in FIG. 22K, a patient with a disease or disorder who could benefit from treatment with a PAX5 or PP1F inhibitor is selected 120. As shown in FIG. 22K, in some embodiments, one or more small molecules 121′ can be administered to the patient to achieve a desired therapeutic result (e.g., lowering the occurrence of an ARD) 121.

In some embodiments, as shown in FIG. 22L, alternatively, multiple therapies can be used in conjunction to achieve a desired therapeutic results. In some embodiments, as shown in FIG. 22K, a patient with a disease or disorder who could benefit from treatment with a PAX5 or PP1F inhibitor is selected 120. In some embodiments, as shown, a cell is acquired and treated by exposing it to one or more different PAX5 gene RNAi(s) and/or PPM1G gene RNAi(s) 111′, 111″. In some embodiments, as shown, the RNAi(s) are allowed to act on the cell for a period of time 112. In some embodiments, as shown, this results in a target cell 113. In some embodiments, as shown in FIG. 22L, the cell can be reintroduced to the patient (e.g., where it was initially isolated from the patient). In some embodiments, a small molecule PAX5 and/or PP1F inhibitor 121′ is also administered to the patient. In some embodiments, as shown, the patient is thereby treated 122.

Alternatively, in some embodiments, as shown in FIG. 22M, treatment is performed entirely in vivo. In some embodiments, a patient with a disease or disorder who could benefit from treatment with a PAX5 or PP1F inhibitor and/or an antagonist of PAX5 and/or PP1F is selected 120. In some embodiments, as shown, one or more different PAX5 gene RNAi(s) and/or PPM1G gene RNAi(s) 111′, 111″ are administered to the patient. In some embodiments, a small molecule PAX5 and/or PP1F inhibitor 121′ is also administered to the patient. In some embodiments, as shown, the patient is thereby treated 123.

In some embodiments, the polycyclic aromatic small molecule capable of targeting and reducing the activity of BSAP may be termed a BSAP small molecule inhibitor (BAP-MI). In some embodiments, the method of decreasing the extent of cellular senescence occurring in the lymphohematopoietic system of a subject and/or treating an ARD disclosed herein comprises administering to a subject in need thereof an effective amount of a composition comprising a BAP-MI.

In some embodiments, the polycyclic aromatic small molecule may target one or more gene products wherein the gene products are endogenous to the subject. As used herein with respect to small molecules, the term “target” refers to action of a ligand (e.g., small molecule) recognizing, associating with, and/or binding to a target molecule (i.e., substrate) that is targeted by the ligand. In a further aspect, the polycyclic aromatic small molecule may reduce the activity of the one or more gene products relative to the activity of the gene products in an absence of the polycyclic aromatic small molecule. Non-limiting examples of gene products suitable for use as described herein (e.g., suitable for use as targets for the polycyclic aromatic small molecule to reduce the activity thereof) are the B-cell lineage specific activator protein (BSAP) (including the protein encoded by the PAX5 gene), the protein phosphatase 1F enzyme (PP1F), or both. In a further aspect, the method of decreasing the extent of cellular senescence occurring in the lymphohematopoietic system of a subject and/or treating an ARD disclosed herein comprises targeting and reducing the activity of BSAP and/or PP1F via administration of an effective amount of the polycyclic aromatic small molecule to a subject in need thereof.

In some embodiments, inhibition of the protein includes reducing its function by greater than or at least about: 1%, 5%, 10%, 30%, 40%, 50%, 60%, 70%, 80%, 90%, 95%, 98%, 99%, 99.9%, 100%, or ranges including and/or spanning the aforementioned values. In some embodiments, a reduction of function includes at least one of decreasing the amount of native protein synthesized, decreasing the full length protein synthesized, decreasing the amount of functional protein synthesized, decreasing the amount of functional fragments of protein synthesized, and decreasing the amount of fragments of protein synthesized. Unless otherwise noted, reduced expression will denote a reduction of the synthesis of the functional protein. In some embodiments, markers for inhibition can include the monitoring protein expression of a molecule that is directly regulated by the target. In some embodiments, PAX5 inhibition can be measured by measuring the amount of p53 synthesized. In some embodiments, inhibition of the PAX5 protein includes reducing transcription of p53 by equal to or at least about: 1%, 5%, 10%, 30%, 40%, 50%, 60%, 70%, 80%, 90%, 95%, 98%, 99%, 99.9%, 100%, or ranges including and/or spanning the aforementioned values. In some embodiments, PPM1F inhibition can be measured by measuring the amount of CaMK2G synthesized. In some embodiments, inhibition of the PPM1F protein includes reducing transcription of CaMK2G by equal to or at least about: 1%, 5%, 10%, 30%, 40%, 50%, 60%, 70%, 80%, 90%, 95%, 98%, 99%, 99.9%, 100%, or ranges including and/or spanning the aforementioned values.

In some embodiments, in using a PAX5 protein inhibitor will reduce the function of one or more proteins including one or more of SEQ ID NO:22 (PAX5 protein; Homo sapiens—as shown in FIG. 22P22), SEQ ID NO:23 (PAX5 protein; Equus caballus—as shown in FIG. 22P23), SEQ ID NO:24 (PAX5 protein; Canis lupus—as shown in FIG. 22P24), SEQ ID NO:25 (PAX5 protein; Felis catus—as shown in FIG. 22P25), SEQ ID NO:26 (PP1F protein; Homo sapiens—as shown in FIG. 22P26), SEQ ID NO:27 (PP1F protein; Equus caballus—as shown in FIG. 22P27), SEQ ID NO:28 (PP1F protein; Canis lupus—as shown in FIG. 22P28), SEQ ID NO:29 (PP1F protein; Felis catus—as shown in FIG. 22P29), and SEQ ID NO:30 (calcium/calmodulin-dependent protein kinase; Homo sapiens—as shown in FIG. 22P30).

The BAP-MI suitable for use in the present disclosure may be any small molecule capable of targeting BSAP. In some embodiments, the BAP-MI may be a polycyclic arene, a polycyclic heteroarene, or combinations thereof; alternatively, a polycyclic arene; or alternatively, a polycyclic heteroarene. In yet a further aspect, the BAP-MI of the present disclosure may be a 6,7-dihydro-5H-benzoheptalen-9-one, a benzoimidazole, or combinations thereof; alternatively, a 6,7-dihydro-5H-benzoheptalen-9-one; or alternatively, a benzoimidazole.

In a particular aspect, the BAP-MI suitable for use in the present disclosure may comprise a member of the class of compounds known as mitotic inhibitors (mitogen spindle inhibitors). A mitotic inhibitor interferes with mitosis (i.e., cell division) by disrupting polymerization of microtubules, which are polymeric forms of the protein tubulin. Microtubules extend through the cell and facilitate the movement of cellular components, e.g., separation of chromosomes and other components of the cell before and during mitosis. Mitotic inhibitors interfere with the assembly and disassembly of tubulin into microtubules and thus interrupt cell division, usually during the mitosis (M) phase of the cell cycle. In some embodiments, the mitotic inhibitor suitable for use as a BSAP small molecule inhibitor may be colcemid (or demecolcine), colchicine, docetaxel, nocodazole, griseofulvin, paclitaxel, vinblastine, vincristine, vinorelbine, any analog or derivative thereof, or combinations thereof. In a further aspect, the mitotic inhibitor suitable for use as a BSAP small molecule inhibitor may be an ansamitocin, campothecin, a combretastatin, a cryptophycin, a curacin, cytochalasin B, discodermolide, a dolastatin, eleutherobin, epothilone A, epothilone B, a flavanol, a halichondrin, a halistatin, lysophosphatidic acid, a maytansinoid, phomopsin A, rhizoxin, a sarcodictyin, a spongistatin, steganacin, a subtilisin, any analog or derivative thereof, or combinations thereof. In a particular aspect, the mitotic inhibitor suitable for use as a BSAP small molecule inhibitor may be colcemid (or demecolcine), nocodazole, any analog or derivative thereof, or combinations thereof.

The BSAP small molecule inhibitors disclosed herein may have Structure BAP-MI 1, Structure BAP-MI 2, Structure Formula I (as disclosed elsewhere herein), Structure Formula II (as disclosed elsewhere herein), Structure Formula III (as disclosed elsewhere herein), or combinations thereof; alternatively, Structure BAP-MI 1; alternatively, Structure BAP-MI 2; alternatively, Structure Formula I; alternatively, Structure Formula II; alternatively, Structure Formula III.

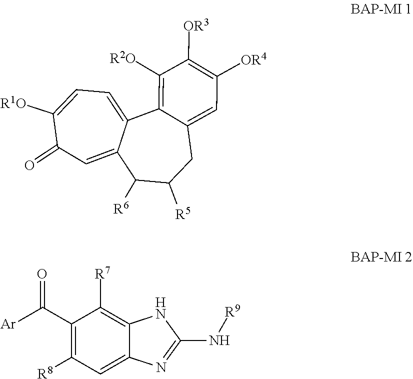

R¹, R², R³, R⁴, R⁵, and R⁶ of the BSAP small molecule inhibitor having Structure BAP-MI 1 are independent elements of the BSAP small molecule inhibitor having Structure BAP-MI 1 and are independently described herein. The independent descriptions of R¹, R², R³, R⁴, R⁵, and R⁶ can be utilized without limitation, and in any combination, to further describe the BSAP small molecule inhibitor having Structure BAP-MI 1. Similarly, Ar, R⁷, R⁸, and R⁹ of the BSAP small molecule inhibitor having Structure BAP-MI 2 are independent elements of the BSAP small molecule inhibitor having Structure BAP-MI 2 and are independently described herein. The independent descriptions of Ar, R⁷, R⁸, and R⁹ can be utilized without limitation, and in any combination, to further describe the BSAP small molecule inhibitor having Structure BAP-MI 2.

Generally, R¹, R², R³, R⁴, R⁷, and/or R⁸ of the respective BSAP small molecule inhibitors, which have an R¹, R², R³, R⁴, R⁷, and/or R⁸ each independently can be hydrogen, an organyl group, a hydrocarbyl group or an aromatic group; alternatively, hydrogen; alternatively, an organyl group; alternatively, a hydrocarbyl group; or alternatively, an aromatic group. In some embodiments, R¹, R², R³, R⁴, R⁷, and/or R⁸, independently can be a C₁ to C₃₀ organyl group; alternatively, a C₁ to C₂₀ organyl group; alternatively, a C₁ to C₁₅ organyl group; alternatively, a C₁ to C₁₀ organyl group; or alternatively, a C₁ to C₅ organyl group. In some embodiments, R¹, R², R³, R⁴, R⁷, and/or R⁸ each independently can be a C₁ to C₃₀ hydrocarbyl group; alternatively, a C₁ to C₂₀ hydrocarbyl group; alternatively, a C₁ to C₁₅ hydrocarbyl group; alternatively, a C₁ to C₁₀ hydrocarbyl group; or alternatively, a C₁ to C₅ hydrocarbyl group. In yet other aspects, R¹, R², R³, R⁴, R⁷, and/or R⁸ each independently can be a C₃ to C₃₀ aromatic group; alternatively, a C₃ to C₂₀ aromatic group; alternatively, a C₃ to C₁₅ aromatic group; or alternatively, a C₃ to C₁₀ aromatic group.

In an aspect R¹, R², R³, R⁴, R⁷, and/or R⁸ each independently can be a C₁ to C₃₀ alkyl group, a C₄ to C₃₀ cycloalkyl group, a C₄ to C₃₀ substituted cycloalkyl group, a C₃ to C₃₀ heterocyclic group, a C₃ to C₃₀ substituted heterocyclic group, a C₆ to C₃₀ aryl group, a C₆ to C₃₀ substituted aryl group, a C₃ to C₃₀ heteroaryl group, or a C₃ to C₃₀ substituted heteroaryl group; alternatively, a C₁ to C₃₀ alkyl group; alternatively, a C₄ to C₃₀ cycloalkyl group; alternatively, a C₄ to C₃₀ substituted cycloalkyl group; alternatively, a C₃ to C₃₀ heterocyclic group; alternatively, a C₃ to C₃₀ substituted heterocyclic group; alternatively, a C₆ to C₃₀ aryl group; alternatively, a C₆ to C₃₀ substituted aryl group; alternatively, a C₃ to C₃₀ heteroaryl group; or alternatively, a C₃ to C₃₀ substituted heteroaryl group. In a further aspect R¹, R², R³, R⁴, R⁷, and/or R⁸ each independently can be a C₁ to C₁₅ alkyl group, a C₄ to C₁₅ cycloalkyl group, a C₄ to C₁₅ substituted cycloalkyl group, a C₃ to C₁₅ heterocyclic group, a C₃ to C₁₅ substituted heterocyclic group, a C₆ to C₁₅ aryl group, a C₆ to C₁₅ substituted aryl group, a C₃ to C₁₅ heteroaryl group, or a C₃ to C₁₅ substituted heteroaryl group; alternatively, a C₁ to C₁₅ alkyl group; alternatively, a C₄ to C₁₅ cycloalkyl group; alternatively, a C₄ to C₁₅ substituted cycloalkyl group; alternatively, a C₃ to C₁₅ heterocyclic group; alternatively, a C₃ to C₁₅ substituted heterocyclic group; alternatively, a C₆ to C₁₅ aryl group; alternatively, a C₆ to C₁₅ substituted aryl group; alternatively, a C₃ to C₁₅ heteroaryl group; or alternatively, a C₃ to C₁₅ substituted heteroaryl group. In a particular aspect R¹, R², R³, R⁴, R⁷, and/or R⁸ each independently can be a C₁ to C₆ alkyl group, a C₄ to C₆ cycloalkyl group, a C₄ to C₆ substituted cycloalkyl group, a C₃ to C₆ heterocyclic group, a C₃ to C₆ substituted heterocyclic group, a C₆ to C₈ aryl group, a C₆ to C₈ substituted aryl group, a C₃ to C₆ heteroaryl group, or a C₃ to C₆ substituted heteroaryl group; alternatively, a C₁ to C₆ alkyl group; alternatively, a C₄ to C₆ cycloalkyl group; alternatively, a C₄ to C₆ substituted cycloalkyl group; alternatively, a C₃ to C₆ heterocyclic group; alternatively, a C₃ to C₆ substituted heterocyclic group; alternatively, a C₆ to C₈ aryl group; alternatively, a C₆ to C₈ substituted aryl group; alternatively, a C₃ to C₆ heteroaryl group; or alternatively, a C₃ to C₆ substituted heteroaryl group.

The non-hydrogen substituents of any substituted R¹, R², R³, R⁴, R⁷, and/or R⁸ group independently can be a hydrocarbyl group. In some embodiments, each non-hydrogen substituent of any substituted R¹, R², R³, R⁴, R⁷, and/or R⁸ group independently can be a halide, a C₁ to C₁₀ hydrocarbyl group, or a C₁ to C₁₀ hydrocarboxy group. In some embodiments, each halide substituent for any substituted R¹, R², R³, R⁴, R⁷, and/or R⁸ group independently can be fluoride, chloride, bromide, or iodide; alternatively, fluoride or chloride.

Generally, R⁵ and/or R⁶ each independently can be a C₁ to C₃₀ hydrocarbyl aminyl group, a C₄ to C₃₀ cycloaminyl group, or a C₄ to C₃₀ substituted cycloaminyl group. In a further aspect, R⁵ and/or R⁶ each independently can be a C₁ to C₁₅ hydrocarbyl aminyl group, a C₄ to C₁₅ cycloaminyl group, or a C₄ to C₁₅ substituted cycloaminyl group. In still a further aspect, R⁵ and/or R⁶ each independently can be a C₁ to C₅ hydrocarbyl aminyl group, a C₄ to C₅ cycloaminyl group, or a C₄ to C₅ substituted cycloaminyl group. In some embodiments, each hydrocarbyl group of a hydrocarbyl aminyl group can be a C₁ to C₃₀ hydrocarbyl group; alternatively, a C₁ to C₁₅ hydrocarbyl group; or alternatively, a C₁ to C₅ hydrocarbyl group.

In some embodiments, Ar can be a pyridinyl group, a substituted pyridinyl group, a furyl group, a substituted furyl group, a thienyl group, or a substituted thienyl group. In some embodiments, the furyl (or substituted furyl) Ar group can be a fur-2-yl group, a substituted fur-2-yl group, a fur-3-yl group, or a substituted fur-3-yl group. In some embodiments, the thienyl (or substituted thienyl) Ar group be a thien-2-yl group, a substituted thien-2-yl group, a thien-3-yl group, or a substituted thien-3-yl group. Substituents for the substituted furyl groups and substituted thienyl groups are independently disclosed herein and can be utilized without limitation to further describe the substituted furyl groups and substituted thienyl groups which can be utilized as Ar. In some embodiments, each substituent for a substituted pyridinyl, furyl, and/or thienyl group that can be independently utilized as Ar can be a halogen, a hydrocarbyl group, or a hydrocarboxy group. In some aspects, each substituent for a substituted pyridinyl, furyl, and/or thienyl group can be a halogen, an alkyl group, or an alkoxy group.

In a particular aspect, R⁹ may be represented by formula COOR¹⁰ or by formula C(O)R¹¹; alternatively, by formula COOR¹⁰; or alternatively by formula C(O)R¹¹. In a further aspect R¹⁰ may be hydrogen, a C₁ to C₁₂ alkyl group, a C₃ to C₁₀ cycloalkyl group, a C₆ to C₁₂ aralkyl group, a phenyl group, or substituted phenyl group. In a further aspect, R¹¹ may be an amino group of formula N(R¹²)₂, wherein each R¹² independently may be hydrogen, a C₁ to C₁₂ alkyl group, a C₃ to C₁₀ cycloalkyl group, a C₆ to C₁₂ aralkyl group, a phenyl group, a substituted phenyl group, a pyridyl group, a substituted pyridyl group, a C₁ to C₅ hydroxyalkyl group, or a C₁ to C₄ dihydroxyalkyl group. In a further aspect, R¹¹ may be a cycloamino group selected from the group consisting of pyrolidino, piperidino, morpholino, piperazino, hexamethyleneimino, pyrrolino, or 3,4-didehydropiperidinyl optionally substituted by one or more C₁ to C₁₂ alkyl groups. In a still further aspect, R¹¹ may be a carbonylamino of formula NR¹³C(O)R¹², wherein R¹³ is hydrogen or a C₁ to C₄ alkyl group, and R¹² is a C₁ to C₄ alkyl group. In yet a further aspect, R¹¹ may be a sulfonylamino of formula NR¹³SO₂R¹², wherein R¹² and R¹³ may be the same, respectively, as any R¹² and R¹³ previously disclosed herein. In some embodiments, each substituent for a substituted phenyl group (general or specific) or a substituted pyridyl group (general or specific) that can be utilized as R¹⁰ and/or R¹² independently can be a halogen, a hydrocarbyl group, a nitro group or a hydrocarboxy group; alternatively, a halogen; alternatively, a hydrocarbyl group; alternatively, a nitro group; or alternatively, a hydrocarboxy group.

In some embodiments, the polycyclic compound is of formula I:

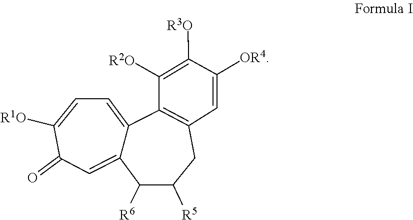

In some embodiments, each of R₁, R₂, R₃, R₄, R₅, and R₆ is independently selected from —H, hydroxyl, halogen, C₁ to C₆ alkyl optionally substituted with halogen or hydroxy, optionally substituted C₁ to C₆ alkenyl, optionally substituted C₁ to C₆ alkynyl, optionally substituted C₁ to C₆ alkoxy, optionally substituted C₁ to C₆ haloalkyl, optionally substituted C₁ to C₆ haloalkoxy, mono-substituted amine(C₁ to C₆ alkyl optionally substituted), a di-substituted amine(C₁ to C₆ alkyl optionally substituted), a diamino-group, and an optionally substituted polyether—having 1 to 6 repeat units. In some embodiments, each of R₁, R₂, R₃, R₄, R₅, and R₆ is independently selected from —H, hydroxyl, halogen, C₁ to C₆ alkyl optionally substituted with halogen or hydroxy, and a —(OR_{B}—)ₒOH, where R_{B} is an optionally substituted C₁ to C₆ alkyl.

In some embodiments, the polycyclic compound is of formula II:

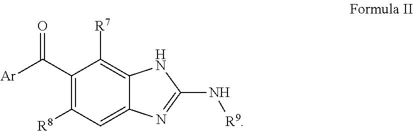

In some embodiments, each of R₇, R₈, and R₉ is independently selected from —H, hydroxyl, halogen, C₁ to C₆ alkyl optionally substituted with halogen or hydroxy, optionally substituted C₁ to C₆ alkenyl, optionally substituted C₁ to C₆ alkynyl, optionally substituted C₁ to C₆ alkoxy, optionally substituted C₁ to C₆ haloalkyl, optionally substituted C₁ to C₆ haloalkoxy, mono-substituted amine(C₁ to C₆ alkyl optionally substituted), a di-substituted amine(C₁ to C₆ alkyl optionally substituted), a diamino-group, and an optionally substituted polyether—having 1 to 6 repeat units. In some embodiments, each of R₇, R₈, and R₉ is independently selected from —H, hydroxyl, halogen, C₁ to C₆ alkyl optionally substituted with halogen or hydroxy, and a —(OR_{B}—)ₒOH, where R_{B} is an optionally substituted C₁ to C₆ alkyl.

In some embodiments, the polycyclic compound is of formula III:

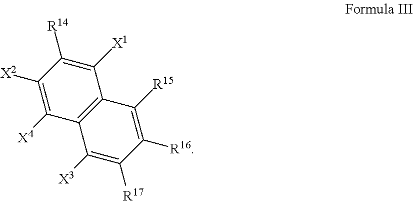

In some embodiments, each of X₁, X₂, X₃, X₄ is independently selected from —H, hydroxyl, halogen, —NH₂, optionally substituted —SO₂OR₁₈. In some embodiments, each of R₁₄, R₁₅, R₁₆, R₁₇, and R₁₈ is independently selected from —H, hydroxyl, halogen, —NH₂, C₁ to C₆ alkyl optionally substituted with halogen or hydroxy, optionally substituted C₁ to C₆ alkenyl, optionally substituted C₁ to C₆ alkynyl, optionally substituted C₁ to C₆ alkoxy, optionally substituted C₁ to C₆ haloalkyl, optionally substituted C₁ to C₆ haloalkoxy, mono-substituted amine(C₁ to C₆ alkyl optionally substituted), a di-substituted amine(C₁ to C₆ alkyl optionally substituted), a diamino-group, and an optionally substituted polyether—having 1 to 6 repeat units. In some embodiments, R₁₄, R₁₅, R₁₆, R₁₇, and R₁₈ are independently selected from —H, hydroxyl, halogen, C₁ to C₆ alkyl optionally substituted with halogen or hydroxy, and a —(OR_{B}—)ₒOH, where R_{B} is an optionally substituted C₁ to C₆ alkyl.

In some embodiments, the compound of formula III is represented by the following structure:

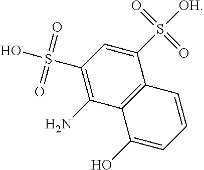 [SMILES: Nc1c(S(=O)(=O)O)cc(S(=O)(=O)O)c2cccc(O)c12]

In some embodiments, the polycyclic compound is provided as a pharmaceutically acceptable salt.

Some embodiments pertain to a pharmaceutical composition comprising one or more polycyclic aromatic compounds and a pharmaceutically acceptable carrier. In some embodiments, the pharmaceutical compound comprises a polycyclic aromatic compound and an RNAi as disclosed elsewhere herein.

Disclosed herein is a composition comprising a polycyclic aromatic small molecule capable of targeting PP1F (e.g., the protein resulting from the PPM1F gene) and reducing the activity of PP1F. In some embodiments, the polycyclic aromatic small molecule capable of targeting and reducing the activity of PP1F may be termed a PP1F small molecule inhibitor (P1F-MI). In some embodiments, the method of decreasing the extent of cellular senescence occurring in the lymphohematopoietic system of a subject and/or treating an ARD disclosed herein comprises administering to a subject in need thereof an effective amount of a composition comprising a P1F-MI.

The P1F-MI suitable for use in the present disclosure may be any small molecule capable of targeting PP1F. In some embodiments, the P1F-MI may be a polycyclic arene, a polycyclic heteroarene, or combinations thereof; alternatively, a polycyclic arene; or alternatively, a polycyclic heteroarene. In yet a further aspect, the P1F-MI of the present disclosure may be a benzophenanthridine, a benzophenanthridinium or combinations thereof; alternatively, a benzophenanthridine; or alternatively, a benzophenanthridinium. In yet a further aspect, the P1F-MI of the present disclosure may be a piperazine bisindole, a pyrazine bisindole, a guanidinium pyrazine bisindole, or combinations thereof; alternatively, a piperazine bisindole; alternatively, a pyrazine bisindole; or alternatively, a guanidinium pyrazine bisindole.

Natural products provide a bountiful source of compounds that potently inhibit the catalytic activity of PP1F. In a particular aspect, the P1F-MI suitable for use in the present disclosure may comprise a sanguinarine salt complex or combinations thereof. Sanguinarine is a polycyclic ammonium ion that is extracted from plants including the bloodroot plant (Sanguinaria canadensis) and the Mexican prickly poppy (Argemone mexicana). In some embodiments, the sanguinarine salt complex suitable for use as the P1F-MI as disclosed herein is represented by Structure P1F-MI 1 wherein X represents a monoanion.

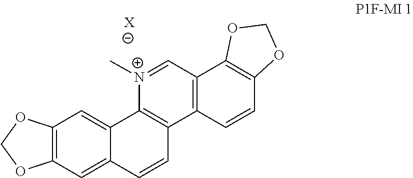

Generally, the monoanion, X, of Structure P1F-MI 1 may be any monoanion suitable for use as a component of the sanguinarine salt complex. In some embodiments, the monoanion, X can be a halide, a carboxylate, a hydrocarboxide, a nitrate, a phosphate, a sulfate, or a chlorate. In a further aspect, the halide suitable for use as X can be fluoride, chloride, bromide, iodide, or any combination thereof; or alternatively, chloride. In a further aspect, the carboxylate suitable for use as X may be acetate, a propionate, trifluoroacetate, or any combination thereof. In a further aspect, the hydrocarboxide suitable for use as X may be an alkoxide, an aryloxide, or an aralkoxide. In a still further aspect, the P1F-MI suitable for use in the present disclosure may comprise sanguinarine chloride or a derivative thereof.

In a particular aspect, the P1F-MI suitable for use in the present disclosure may comprise a member of the dragmacidin family of small molecules or combinations thereof. The dragmacidins represent an emerging class of bioactive marine natural products obtained from a number of deep water sponges including Dragmacidon, Halicortex, Spongosorites, and Hexadella, and the tunicate Didemnum candid. Dragmacidin D, which has been found to serve as a potent inhibitor of serine-threonine protein phosphatases, has received particular attention as a lead compound for treating Parkinson's, Alzheimer's, and Huntington's diseases. In some embodiments, the P1F-MI suitable for use in the present disclosure may comprise dragmacidin A, dragmacidin B, dragmacidin C, dragmacidin D, dragmacidin E, dragmacidin F, or combinations thereof.

In a still further aspect, the PP1F small molecule inhibitor (P1F-MI) disclosed herein may have Structure P1F-MI 2, Structure P1F-MI 3, Structure P1F-MI 4, Structure P1F-MI 5, Structure P1F-MI 6, Structure P1F-MI 7, Structure P1F-MI 8, Structure P1F-MI 9, Structure P1F-MI 10, or combinations thereof; alternatively, Structure P1F-MI 2; alternatively, Structure P1F-MI 3; alternatively, Structure P1F-MI 4; alternatively, Structure P1F-MI 5; alternatively, Structure P1F-MI 6; alternatively, Structure P1F-MI 7; alternatively, Structure P1F-MI 8; alternatively, Structure P1F-MI 9; or alternatively, Structure P1F-MI 10.

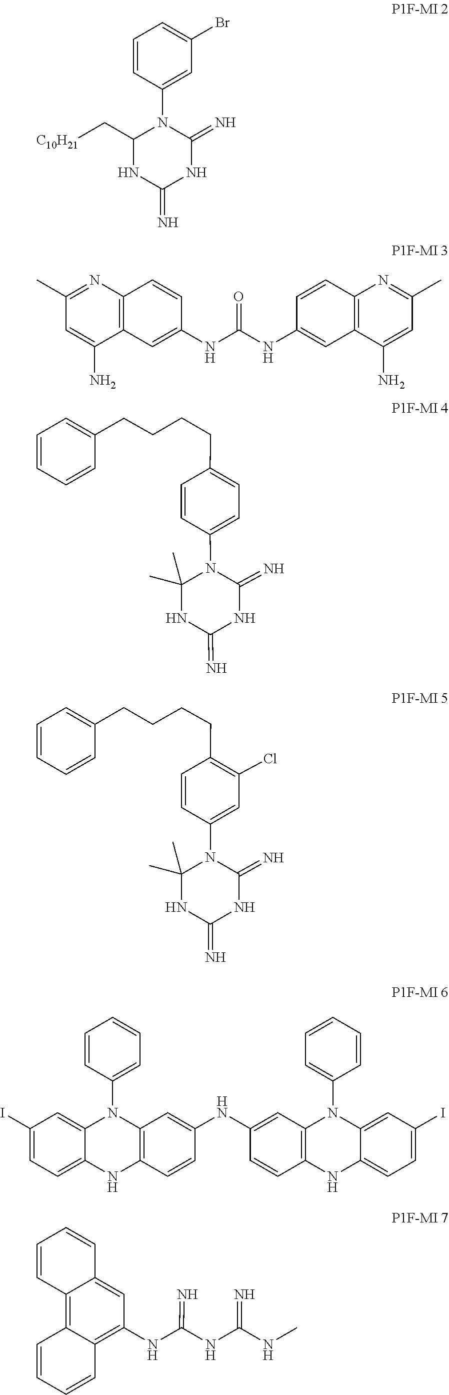

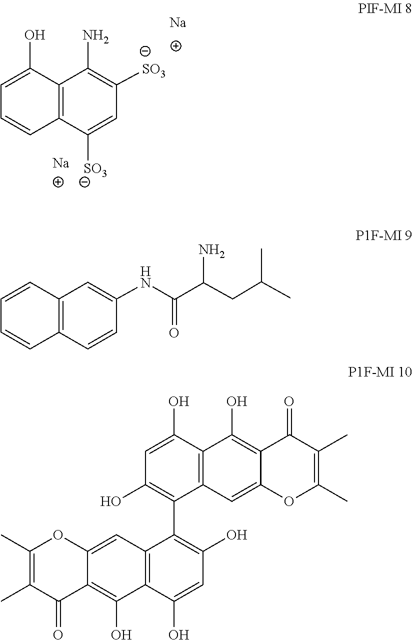

Some embodiments pertain to method for treating or preventing age related dysfunction or other cellular dysfunction, comprising administering to a patient in need thereof a therapeutically effective dose of one or more polycyclic aromatic compounds that antagonize or reduce the expression of PAX5 and/or PPM1F.

Disclosed herein are pharmaceutical formulations of one or more BSAP small molecule inhibitors (BAP-MIs), one or more PP1F small molecule inhibitors (P1F-MIs), or combinations thereof. The BAP-MIs and/or P1F-MIs described herein may be formulated in a variety of manners, and thus may additionally comprise one or more carriers of the type disclosed herein, and it is to be understood that various of the specific carriers disclosed herein may be used in combination. In this regard, a wide variety of carriers may be selected of either polymeric or non-polymeric origin. In one particular aspect, a wide variety of polymeric carriers may be utilized to contain and/or deliver one or more of the BAP-MIs discussed herein, one or more P1F-MIs discussed herein, or combinations thereof, including for example both biodegradable and non-biodegradable compositions. Representative examples of biodegradable compositions include one or more of albumin, collagen, gelatin, hyaluronic acid, starch, cellulose (methylcellulose, hydroxypropylcellulose, hydroxypropylmethylcellulose, hydroxyethylcellulose, carboxymethylcellulose, cellulose acetate phthalate, cellulose acetate succinate, hydroxypropylmethylcellulose phthalate), casein, dextrans, polysaccharides, fibrinogen, poly(D,L lactide), poly(D,L-lactide-co-glycolide), poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids) and their copolymers. Representative examples of nondegradable polymers include one or more of poly(ethylene-vinyl acetate) (“EVA”) copolymers, silicone rubber, acrylic polymers (polyacrylic acid, polymethylacrylic acid, polymethylmethacrylate, polyalkylcynoacrylate), polyethylene, polyproplene, polyamides (nylon 6,6), polyurethane, poly(ester urethanes), poly(ether urethanes), poly(ester-urea), polyethers (poly(ethylene oxide), poly(propylene oxide), PLURONICS® and poly(tetramethylene glycol)), silicone rubbers and vinyl polymers (polyvinylpyrrolidone, poly(vinyl alcohol), poly(vinyl acetate phthalate). Polymers may also be developed which are either anionic (e.g., alginate, carrageenin, carboxymethyl cellulose and poly(acrylic acid), or cationic (e.g., chitosan, poly-L-lysine, polyethylenimine, and poly (allyl amine)). Particularly preferred polymeric carriers include one or more of poly(ethylene-vinyl acetate), poly (D,L-lactic acid) oligomers and polymers, poly (L-lactic acid) oligomers and polymers, poly (glycolic acid), copolymers of lactic acid and glycolic acid, poly (caprolactone), poly (valerolactone), polyanhydrides, copolymers of poly (caprolactone) or poly (lactic acid) with a polyethylene glycol (e.g., MePEG), and blends thereof.

Polymeric carriers can be fashioned in a variety of forms, with desired release characteristics and/or with specific desired properties. For example, polymeric carriers may be fashioned to release a BAP-MI or P1F-MI upon exposure to a specific triggering event such as pH. Representative examples of pH-sensitive polymers include one or more of poly(acrylic acid) and its derivatives (including for example, homopolymers such as poly(aminocarboxylic acid); poly(acrylic acid); poly(methyl acrylic acid), copolymers of such homopolymers, and copolymers of poly(acrylic acid) and acrylmonomers such as those discussed above. Other pH sensitive polymers include one or more polysaccharides such as cellulose acetate phthalate; hydroxypropylmethylcellulose phthalate; hydroxypropylmethylcellulose acetate succinate; cellulose acetate trimellilate; and chitosan. Yet other pH sensitive polymers include any mixture of a pH sensitive polymer and a water-soluble polymer. Likewise, polymeric carriers can be fashioned which are temperature sensitive.

Representative examples of thermogelling polymers, and their gelation temperature (lower critical solution temperature (LCST) (° C.)) include homopolymers such as poly(N-methyl-N-n-propylacrylamide), 19.8° C.; poly(N-n-propylacrylamide), 21.5° C.; poly(N-methyl-N-isopropylacrylamide), 22.3° C.; poly(N-n-propylmethacrylamide), 28.0° C.; poly(N-isopropylacrylamide), 30.9° C.; poly(N, n-diethylacrylamide), 32.0° C.; poly(N-isopropylmethacrylamide), 44.0° C.; poly(N-cyclopropylacrylamide), 45.5° C.; poly(N-ethylmethyacrylamide), 50.0° C.; poly(N-methyl-N-ethylacrylamide), 56.0° C.; poly(N-cyclopropylmethacrylamide), 59.0° C.; and poly(N-ethylacrylamide), 72.0° C. Moreover, thermogelling polymers may be made by preparing copolymers between (among) monomers of the above, or by combining such homopolymers with other water soluble polymers such as acrylmonomers (e.g., acrylic acid and derivatives thereof such as methylacrylic acid, acrylate and derivatives thereof such as butyl methacrylate, acrylamide, and N-n-butyl acrylamide).

Other representative examples of thermogelling polymers (and their gelation temperatures) include cellulose ether derivatives such as hydroxypropyl cellulose, 41° C.; methyl cellulose, 55° C.; hydroxypropylmethyl cellulose, 66° C.; and ethylhydroxyethyl cellulose, and PLURONICS® such as F-127, 10-15° C.; L-122, 19° C.; L-92, 26° C.; L-81, 20° C.; and L-61, 24° C.

A wide variety of forms may be fashioned by the polymeric carriers of the present disclosure, including for example, rod-shaped devices, pellets, slabs, or capsules. The BAP-MIs or P1F-MIs may be linked by occlusion in the matrices of the polymer, bound by covalent linkages, or encapsulated in microcapsules.

Compositions comprising one or more BAP-MIs disclosed herein, one or more P1F-MIs disclosed herein, or combinations thereof may be fashioned in any manner appropriate to the intended use. Within certain aspects, the composition comprising one or more BAP-MIs disclosed herein, one or more P1F-MIs disclosed herein, or combinations thereof should be biocompatible, and release one or more BAP-MIs and/or P1F-MIs over a period of several days to months. For example, “quick release” or “burst” compositions are provided that release greater than 10%, 20%, or 25% (w/v) of a BAP-MI (e.g., colcemid) and/or P1F-MI (e.g., sanguinarine chloride) over a period of 7 to 10 days. Such “quick release” compositions should, within certain aspects, be capable of releasing chemotherapeutic levels (where applicable) of a desired agent. Within other aspects, “low release” therapeutic compositions are provided that release less than 1% (w/v) of a BAP-MI and/or P1F-MI over a period of 7 to 10 days. Further, compositions comprising a BAP-MI and/or P1F-MI as disclosed herein should preferably be stable for several months and capable of being produced and maintained under sterile conditions.

Within further aspects, the compositions comprising one or more BAP-MIs disclosed herein, one or more P1F-MIs disclosed herein, or combinations thereof may be formulated for topical application. Representative examples include: ethanol; mixtures of ethanol and glycols (e.g., ethylene glycol or propylene glycol); mixtures of ethanol and isopropyl myristate or ethanol, isopropyl myristate and water (e.g., 55:5:40); mixtures of ethanol and eineol or D-limonene (with or without water); glycols (e.g., ethylene glycol or propylene glycol) and mixtures of glycols such as propylene glycol and water, phosphatidyl glycerol, dioleoylphosphatidyl glycerol, ethyldiglycol (i.e., TRANSCUTOL®), or terpinolene; mixtures of isopropyl myristate and 1-hexyl-2-pyrrolidone, N-dodecyl-2-piperidinone or 1-hexyl-2-pyrrolidone. Other excipients may also be added to the above, including for example, acids such as oleic acid and linoleic acid, and soaps such as sodium lauryl sulfate. A preferred aspect would include buffered saline or water, antimicrobial agents (e.g., methylparaben, propylparaben), carrier polymer(s), such as celluloses (e.g., hydroxyethylcellulose) and (a) penetration or permeation enhancer(s) (e.g., ethoxydiglycol-TRANSCUTOL®, isopropyl myristate, ethylene glycol, 1-hexyl-2-pyrrolidone, D-limonene).

In a particular aspect, the compositions and methods disclosed herein may provide means of restoring an aging lymphohematopoietic system. Herein reference is made to the undifferentiated cells of the hematopoietic lineage including hematopoietic stem cells (HSCs), lymphoid progenitor cells (LPCs) and myeloid progenitor cells (MPCs) which are known collectively as lymphohaematopoietic progenitor cells (LPCs). LPCs and MPCs are each formed by the differentiation of HSCs.

Other examples of differentiated cells of the hematopoietic lineage include T lymphocytes, B lymphocytes, eosinophils, basophils, neutrophils, megakaryocytes, monocytes, macrophages erythrocytes, granulocytes, mast cells, dendritic cells and natural killer cells. The pathways of differentiation in the lymphohematopoietic system have been extensively characterized and the various cell stages are readily identifiable according to morphology and lineage-specific cell surface markers.

It should be noted that, while some of the results achieved are described as being a result of using either the small molecules disclosed herein or the RNAi(s) disclosed herein, it should be appreciated that the RNAi(s) and small molecules can accomplish one or more results disclosed for the other.

In some embodiments, the present disclosure contemplates the utilization of BAP-MIs and/or P1F-MIs which are hereinafter collectively referred to small inhibitory molecules (SIMs). In some embodiments, SIMs of the type disclosed herein are utilized as compositions for administration to a subject in need thereof. In some embodiments, the SIMs may be a component of a pharmaceutical formulation that is administered locally or systemically to a subject. In some embodiments, SIMs of the type disclosed herein are used in conjunction with a vehicle such as a nanoparticle, micelle, liposome, niosome, microsphere, cyclodextrin and the like. In some embodiments, such vehicles further comprise one or more elements to direct the carrier or vehicle to a particular cell, tissue or organ of a subject (e.g., cells of the lymphohematopoietic system).

Some embodiments pertain to a pharmaceutical composition comprising a polycyclic aromatic small molecule capable of targeting and reducing the activity of the B-cell lineage specific activator protein, the protein phosphatase 1F enzyme, or both when the composition is administered in an effective amount to a subject in need thereof, wherein the B-cell lineage specific activator protein is a gene product of the paired box 5 (PAX5) gene and the protein phosphatase 1F enzyme is a gene product of the PPM1F gene. Some embodiments pertain to a method of treatment comprising administering to a subject in need thereof a therapeutically effective amount of the pharmaceutical composition. In some embodiments, the subject has one or more medical conditions or age-related disorders selected from the group consisting of arthritis, atherosclerosis, breast cancer, cardiovascular disease, cataracts, chronic obstructive pulmonary disease, colorectal cancer, hypertension, osteoporosis, periodontitis, type 2 diabetes, and Alzheimer's disease.

### Enumerated Embodiments

The following provide exemplary illustrative enumerated embodiments:

1. A method of reducing expression of a paired box 5 (PAX5) gene and reducing expression of a protein phosphatase 1F enzyme (PPM1F) gene in a cell, the method comprising: contacting the cell with one or more interfering RNA(s) (RNAi(s)) comprising one or more of SEQ ID NO:15, SEQ ID NO:16, SEQ ID NO:17, SEQ ID NO:18, SEQ ID NO:19, SEQ ID NO:20; and maintaining the cell for a time sufficient to obtain inhibition of the PAX5 gene and the PPM1F gene, thereby reducing expression of the PAX5 gene and the PPM1F gene in that cell to provide a target cell.

2. The method of embodiment 1, wherein the one or more RNAi(s) comprises SEQ ID NO:15.

3. The method of embodiment 1 or 2, wherein the one or more RNAi(s) comprises at least one of SEQ ID NO:15, SEQ ID NO:17, and SEQ ID NO:19.

4. The method of any one of embodiments 1 to 3, wherein the one or more RNAi(s) comprises at least one of SEQ ID NO:16, SEQ ID NO:18, and SEQ ID NO:20.

5. The method of any one of embodiments 1 to 4, wherein the one or more RNAi(s) further comprises at least one of SEQ ID NO:9, SEQ ID NO:10, SEQ ID NO:11, SEQ ID NO:12, SEQ ID NO:13, and SEQ ID NO:14.

6. The method of any one of embodiments 1 to 5, wherein the cell is isolated from a subject.

7. The method of any one of embodiments 1 to 6, wherein the cell is inside a subject.

8. The method of any one of embodiments 1 to 7, wherein the cell is a human cell.

9. The method of any one of embodiments 1 to 8, wherein the PAX5 expression is reduced by at least about 30%, 40%, 50%, 60%, 70%, 80%, 90%, 95%, 98%, 99%, or 99.9%.

10. The method of any one of embodiments 1 to 9, wherein the PAX5 expression is reduced by at least about 70%.

11. The method of any one of embodiments 1 to 10, wherein the PPM1F expression is reduced by at least about 30%, 40%, 50%, 60%, 70%, 80%, 90%, 95%, 98%, 99%, or 99.9%.

12. The method of any one of embodiments 1 to 11, wherein the PPM1F expression is reduced by at least about 70%.

13. The method of any one of embodiments 1 to 12, wherein the cell is contacted with the one or more RNAi(s) for a period of equal to or at least about 16 hours, 48 hours, or 72 hours.

14. A target cell made by the method of any one of embodiments 1 to 13.

15. The target cell of embodiment 14, wherein the target cell is non-senescent and/or has decreased senescent behavior, has increased innate immune function, increased telomere length, lower replicative stress relative to the patient cell, increased stem cell clonogenicity; increased cytotoxic function, increased mitogen- and/or antigen-induced lymphocyte proliferation and/or activation, decreased myeloid to lymphoid ratio, increased CD4 to CD8 T lymphocyte ratio, decreased expression of senescence-associated secretory proteins, and/or decreased expression of senescence- and/or aging-related genes.

16. A composition for reducing expression of a PAX5 gene and reducing the expression of a PPM1F gene, the composition comprising an acceptable carrier and an RNAi that is at least 80% to 100% identical to SEQ ID NO:15.

17. The composition of embodiment 16, further comprising a microRNA that is at least 80% to 100% identical to SEQ ID NO:16.

18. The composition of embodiment 16 or 17, further comprising a microRNA that is at least 80% to 100% identical to SEQ ID NO:17.

19. The composition of any one of embodiments 16 to 18, further comprising a microRNA that is at least 80% to 100% identical to SEQ ID NO:18.

20. The composition of any one of embodiments 16 to 19, further comprising a microRNA that is at least 80% to 100% identical to SEQ ID NO:19.

21. The composition of any one of embodiments 16 to 20, further comprising a microRNA that is at least 80% to 100% identical to SEQ ID NO:20.

22. A method of treating a subject having a disease or disorder that would benefit from reduction in expression of a PAX5 gene and a reduction in expression of a PPM1F gene, the method comprising administering to the subject a therapeutically effective amount of the target cell of any one of embodiments 1 to 15 or the composition of any one of embodiments 16 to 21, thereby treating the subject.

23. An interfering RNA (RNAi) for reducing the expression of a paired box 5 (PAX5) gene, wherein the RNAi comprises 4 to 50 contiguous nucleotides having a polynucleotide sequence that is at least 80% to 100% complementary to a region of SEQ ID NO:1.

24. The RNAi of embodiment 23, wherein the RNAi is a short interfering RNA (siRNA), microRNA (miRNA), circular RNAs (circRNAs), short hairpin RNAs (shRNAs), long non-coding RNAs (lncRNAs); piwi-interacting RNAs (piRNA), small nucleolar RNA (snoRNAs), tRNA-derived small RNA (tsRNA), small rDNA-derived RNA (srRNA), or a small nuclear RNA (U-RNA).

25. The RNAi of embodiment 23, wherein the RNAi is an siRNA.

26. The RNAi of any one of embodiments 23 to 25, wherein the RNAi hybridizes to the complimentary region of SEQ ID NO:1.

27. The RNAi of embodiment any one of embodiments 23 to 26, wherein the RNAi comprises about 20 to 30 contiguous nucleotides.

28. The RNAi of any one of embodiments 23 to 27, wherein the RNAi is at least 80% to 100% identical to the nucleotide sequence of SEQ ID NO:9.

29. The RNAi of any one of embodiments 23 to 27, wherein the RNAi is at least 80% to 100% identical to the nucleotide sequence of SEQ ID NO:10.

30. The RNAi of any one of embodiments 23 to 27, wherein the RNAi is at least 80% to 100% identical to the nucleotide sequence of SEQ ID NO:11.

31. A composition for reducing expression of a PAX5 gene comprising the RNAi of any one of embodiments 23 to 30.

32. The composition of embodiment 31, wherein the composition comprises two or more of SEQ ID NO:9, SEQ ID NO:10, SEQ ID NO:11, SEQ ID NO:15, SEQ ID NO:16, SEQ ID NO:17, SEQ ID NO:18, SEQ ID NO:19, or SEQ ID NO:20.

33. The composition of embodiment 31 or 32, further comprising a pharmaceutically acceptable carrier.

34. A method of reducing expression of a PAX5 gene in a cell, the method comprising:

contacting the cell with the RNAi of any one of embodiments 1 to 30 or the composition of any one of embodiments 31 to 33; and

maintaining the cell for a time sufficient to obtain inhibition of the PAX5 gene, thereby reducing expression of the PAX5 gene in the cell.

35. The method of embodiment 34, wherein the cell is isolated from or is inside a subject.

36. The method of embodiment 35, wherein the subject is a human.

37. The method of any one of embodiments 34 to 36, wherein the PAX5 expression is reduced by at least about 30%, 40%, 50%, 60%, 70%, 80%, 90%, 95%, 98%, 99%, or 99.9%.

38. The method of any one of embodiments 34 to 36, wherein the PAX5 expression is reduced by at least about 70%.

39. The method of any one of embodiments 34 to 38, wherein the cell is contacted with the RNAi for a period of equal to or at least about 16 hours, 48 hours, or 72 hours.

40. A cell made by the method any one of embodiments 34 to 39.

41. The target cell of embodiment 40, wherein the target cell is non-senescent and/or has decreased senescent behavior, has increased innate immune function, increased telomere length, lower replicative stress relative to the patient cell, increased stem cell clonogenicity; increased cytotoxic function, increased mitogen- and/or antigen-induced lymphocyte proliferation and/or activation, decreased myeloid to lymphoid ratio, increased CD4 to CD8 T lymphocyte ratio, decreased expression of senescence-associated secretory proteins, and/or decreased expression of senescence- and/or aging-related genes.

42. A method of treating a subject having a disease or disorder that would benefit from reduction in expression of a PAX5 gene, the method comprising administering to the subject a therapeutically effective amount of cells that have been treated with the RNAi of any one of embodiments 1 to 30 or the composition of any one of embodiments 31 to 33, thereby treating the subject.

43. An RNAi for reducing the expression of a protein phosphatase 1F enzyme (PPM1F) gene, wherein the RNAi comprises 4 to 50 contiguous nucleotides having a polynucleotide sequence that is at least 80% to 100% complementary to a region of SEQ ID NO:5.

44. The RNAi of embodiment 43, wherein the interfering RNA is a short interfering RNA (siRNA).

45. The RNAi of embodiment 43 or 44, wherein the RNAi hybridizes to the complimentary region of SEQ ID NO:1.

46. The RNAi of any one of embodiments 43 to 45, wherein the RNAi comprises about 20 to 30 contiguous nucleotides.

47. The RNAi of any one of embodiments 43 to 46, wherein the RNAi is at least 80% to 100% identical to the nucleotide sequence of SEQ ID NO:12.

48. The RNAi of any one of embodiments 43 to 46, wherein the RNAi is at least 80% to 100% identical to the nucleotide sequence of SEQ ID NO:13.

49. The RNAi of any one of embodiments 43 to 46, wherein the RNAi is at least 80% to 100% identical to the nucleotide sequence of SEQ ID NO:14.

50. A composition for reducing expression of a PPM1F gene comprising the RNAi of any one of embodiments 43 to 49.

51. The composition of embodiment 50, wherein the composition comprises two or more of SEQ ID NO:12, SEQ ID NO:13, SEQ ID NO:14, SEQ ID NO:15, SEQ ID NO:16, SEQ ID NO:17, SEQ ID NO:18, SEQ ID NO:19, or SEQ ID NO:20.

52. The composition of embodiment 50 or 51, further comprising a pharmaceutically acceptable carrier.

53. The composition of embodiment 52, wherein the pharmaceutically acceptable carrier comprises one or more of nanoparticles composed of non-degradable and/or degradable biomaterials, micelles, liposomes, extracellular vesicles (native and/or synthetic), exosomes (native and/or synthetic), and/or microvesicles (native and/or synthetic).

54. A method of reducing expression of a PPM1F gene in a cell, the method comprising: contacting the cell with the RNAi of any one of embodiments 43 to 49 or the composition of any one of embodiments 50 to 52; and maintaining the cell for a time sufficient to obtain inhibition of the PPM1F gene, thereby reducing expression of the PPM1F gene in the cell.

55. The method of embodiment 54, wherein the cell is isolated from or is inside a subject.

56. The method of embodiment 55, wherein the subject is a human.

57. The method of any one of embodiments 52 to 56, wherein the PPM1F expression is reduced by at least about 30%, 40%, 50%, 60%, 70%, 80%, 90%, 95%, 98%, 99%, or 99.9%.

58. A cell made by the method any one of embodiments 54 to 57.

59. The target cell of embodiment 58, wherein the target cell is non-senescent or has decreased senescent behavior, has increased innate immune function, increased telomere length, lower replicative stress relative to the patient cell, increased stem cell clonogenicity, increased cytotoxic function, increased mitogen- and antigen-induced lymphocyte proliferation and activation, decreased myeloid to lymphoid ratio, increased CD4 to CD8 T lymphocyte ratio, decreased expression of senescence-associated secretory proteins, and/or decreased expression of senescence- and aging-related genes.

60. A method of treating a subject having a disease or disorder that would benefit from reduction in expression of a PPM1F gene, the method comprising administering to the subject a therapeutically effective amount of cells that have been treated with the RNAi of any one of embodiments 43 to 49 or the composition of any one of embodiments 50 to 52, thereby treating the subject.

61. A method for treating or preventing a disease state, comprising administering to a patient in need thereof a therapeutically effective dose of cells treated with one or more RNAi(s) of a PAX5 gene and/or of a PPM1F gene.

62. The method of embodiment 61, wherein the one or more RNAi(s) is selected from any one or more of the RNAis as recited in any one of the preceding embodiments.

63. The method of embodiment 61 or 62, wherein the disease state is an age related dysfunction.

64. The method of any one of embodiments 61 to 63, wherein the disease state comprises one or more of arthritis, atherosclerosis, breast cancer, cardiovascular disease, cataracts, chronic obstructive pulmonary disease, colorectal cancer, hypertension, osteoporosis, periodontitis, type 2 diabetes, immune dysfunction, Alzheimer's disease, leukemia, lymphoma, multiple sclerosis, Crohn's disease, HIV, influenza, pneumonia, lung cancer, melanoma, stroke, Parkinson's disease, and multiple drug resistant Staphylococcus aureus (MRSA).

65. A method for preparing a target cell comprising: obtaining cells from a subject to provide at least one subject cell; exposing the at least one subject cell to one or more RNAis as recited in any one of the preceding embodiments to provide at least one target cell.

66. The method of embodiment 65, wherein the at least one target cell is member of a population of cells comprising equal to or at least about 100, 1000, 10,000, 100,000, 1,000,000, or 10,000,000 cells.

67. A method for treating or preventing cellular dysfunction in a patient, comprising administering to a patient in need thereof a therapeutically effective dose of the target cell of embodiment 65 or 66.

68. The method of embodiment 67, wherein the cellular dysfunction is an age-related dysfunction.

69. A cell made by the method of 65 or 66.

70. An interfering RNA (RNAi) for reducing the expression of a paired any one of the CAMK2G/CAMK-II, PAK, C21orf62-AS1, CASP14, CATSPER2, DNAH10OS, ELMOD1, GALNT6, HEPN1, LANCL2, LL22NC03-63E9.3, PPTC7, PROSC, RAB3B, RRP7A, SERFiA/SERFiB, SLC35E3, SMIM10, SPRY3, SUMO2, TPP1, TPPP, WBP1L, ZNF33A, or ZNF549 gene, wherein the RNAi comprises 4 to 50 contiguous nucleotides having a polynucleotide sequence that is at least 80% to 100% complementary to a region of any one or more of SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20.

71. A method of reducing expression of a paired box 5 (PAX5) gene in a cell, the method comprising: contacting the cell with one or more interfering RNA(s) (RNAi(s)) wherein the one or more RNAi(s) comprises one or more of SEQ ID NO:9, SEQ ID NO:10, SEQ ID NO:11, SEQ ID NO:15, SEQ ID NO:16, SEQ ID NO:17, SEQ ID NO:18, SEQ ID NO:19, SEQ ID NO:20; and maintaining the cell for a time sufficient to obtain inhibition of the PAX5 gene, thereby reducing expression of the PAX5 gene in that cell to provide a target cell.

72. The method of embodiment 71, wherein the one or more RNAi(s) comprises at least one of SEQ ID NO:9, SEQ ID NO:10, and SEQ ID NO:11.

73. The method of embodiment 71, wherein the one or more RNAi(s) comprises at least one of SEQ ID NO:15, SEQ ID NO:17, and SEQ ID NO:19.

74. The method of embodiment 71, wherein the one or more RNAi(s) comprises at least one of SEQ ID NO:16, SEQ ID NO:18, and SEQ ID NO:20.

75. The method of embodiment 71, wherein the cell is isolated from a subject or is inside the subject.

76. The method of embodiment 75, wherein the subject is a human.

77. The method of embodiment 71, wherein the PAX5 expression is reduced by at least about 30%, 40%, 50%, 60%, 70%, 80%, 90%, 95%, 98%, 99%, or 99.9%.

78. The method of embodiment 71, wherein the PAX5 expression is reduced by at least about 70%.

79. The method of embodiment 71, wherein the cell is contacted with the one or more RNAi(s) for a period of equal to or at least about 16 hours, 48 hours, or 72 hours.

80. A target cell made by the method embodiment 71.

81. The target cell of embodiment 80, wherein the target cell is non-senescent and/or has decreased senescent behavior, has increased innate immune function, increased telomere length, lower replicative stress relative to the patient cell, increased stem cell clonogenicity; increased cytotoxic function, increased mitogen- and/or antigen-induced lymphocyte proliferation and/or activation, decreased myeloid to lymphoid ratio, increased CD4 to CD8 T lymphocyte ratio, decreased expression of senescence-associated secretory proteins, and/or decreased expression of senescence- and/or aging-related genes.

82. A method of treating a subject having a disease or disorder that would benefit from reduction in expression of a PAX5 gene, the method comprising administering to the subject a therapeutically effective amount of one or more RNAi(s) selected from the group consisting of SEQ ID NO:9, SEQ ID NO:10, SEQ ID NO:11, SEQ ID NO:15, SEQ ID NO:16, SEQ ID NO:17, SEQ ID NO:18, SEQ ID NO:19, and SEQ ID NO:20 or a therapeutically effective amount of cells that have been treated with the one or more RNAi(s), thereby treating the subject.

83. A method of reducing expression of a protein phosphatase 1F enzyme (PPM1F) gene in a cell, the method comprising: contacting the cell with one or more interfering RNA(s) (RNAi(s)) wherein the one or more RNAi(s) comprises one or more of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20; and maintaining the cell for a time sufficient to obtain inhibition of the PAX5 gene, thereby reducing expression of the PAX5 gene in that cell to provide a target cell.

84. The method of embodiment 83, wherein the one or more RNAi(s) comprises at least one of SEQ ID NO:12, SEQ ID NO:13, and SEQ ID NO:14.

85. The method of embodiment 83, wherein the one or more RNAi(s) comprises at least one of SEQ ID NO:15, SEQ ID NO:17, and SEQ ID NO:19.

86. The method of embodiment 83, wherein the one or more RNAi(s) comprises at least one of SEQ ID NO:16, SEQ ID NO:18, and SEQ ID NO:20.

87. The method of embodiment 83, wherein the cell is isolated from a subject or is inside the subject.

88. The method of embodiment 87, wherein the subject is a human.

89. The method of embodiment 83, wherein the PPM1F expression is reduced by at least about 30%, 40%, 50%, 60%, 70%, 80%, 90%, 95%, 98%, 99%, or 99.9%.

90. The method of embodiment 83, wherein the PPM1F expression is reduced by at least about 70%.

91. The method of embodiment 83, wherein the cell is contacted with the one or more RNAi(s) for a period of equal to or at least about 16 hours, 48 hours, or 72 hours.

92. A target cell made by the method embodiment 83.

93. The target cell of embodiment 92, wherein the target cell is non-senescent and/or has decreased senescent behavior, has increased innate immune function, increased telomere length, lower replicative stress relative to the patient cell, increased stem cell clonogenicity; increased cytotoxic function, increased mitogen- and/or antigen-induced lymphocyte proliferation and/or activation, decreased myeloid to lymphoid ratio, increased CD4 to CD8 T lymphocyte ratio, decreased expression of senescence-associated secretory proteins, and/or decreased expression of senescence- and/or aging-related genes.

94. A method of treating a subject having a disease or disorder that would benefit from reduction in expression of a PPM1F gene, the method comprising administering to the subject a therapeutically effective amount of one or more RNAi(s) selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, and SEQ ID NO:20 or a therapeutically effective amount of cells that have been treated with the one or more RNAi(s), thereby treating the subject.

95. A method for reducing the expression of a PAX5 gene, comprising exposing a cell to a composition comprising at least one isolated microRNA, wherein the at least one microRNA is at least 80% to 100% identical to SEQ ID NO:15.

96. A method for reducing the expression of a PAX5 gene, comprising exposing a cell to a composition comprising at least one isolated microRNA, wherein the at least one microRNA is at least 80% to 100% identical to SEQ ID NO:16.

97. The method of embodiment 95 or 96, wherein the composition comprises both SEQ ID NO: 15 and SEQ ID NO: 16.

98. The method of any one of embodiments 95 to 97, wherein the composition further comprises a microRNA that is at least 80% to 100% identical to SEQ ID NO:17.

99. The method of any one of embodiments 95 to 98, wherein the composition further comprises a microRNA that is at least 80% to 100% identical to SEQ ID NO:18.

100. The method of any one of embodiments 95 to 99, wherein the composition further comprises a microRNA that is at least 80% to 100% identical to SEQ ID NO:19.

101. The method of any one of embodiments 95 to 100, wherein the composition further comprises a microRNA that is at least 80% to 100% identical to SEQ ID NO:20.

102. A composition for reducing expression of a PAX5 gene comprising an acceptable carrier and at least one isolated microRNA that is at least 80% to 100% identical to SEQ ID NO:15.

103. The composition of embodiment 102, further comprising a microRNA that is at least 80% to 100% identical to SEQ ID NO:16.

104. The composition of embodiment 102 or 103, further comprising a microRNA that is at least 80% to 100% identical to SEQ ID NO:17.

105. The composition of any one of embodiments 102 to 104, further comprising a microRNA that is at least 80% to 100% identical to SEQ ID NO:18.

106. The composition of any one of embodiments 102 to 105, further comprising a microRNA that is at least 80% to 100% identical to SEQ ID NO:19.

107. The composition of any one of embodiments 102 to 106, further comprising a microRNA that is at least 80% to 100% identical to SEQ ID NO:20.

108. A method of reducing expression of a PAX5 gene in a cell, the method comprising: contacting the cell with the composition of any one of embodiments 102 to 104; and maintaining the cell for a time sufficient to obtain inhibition of a PAX5 gene, thereby reducing expression of the PAX5 gene in the cell.

109. The method of embodiment 108, wherein the cell is isolated from a subject.

110. The method of embodiment 109, wherein the subject is a human.

111. The method of any one of embodiments 108 to 110, wherein the PAX5 expression is reduced by at least about 30%, 40%, 50%, 60%, 70%, 80%, 90%, 95%, 98%, 99%, or 99.9%.

112. A method of treating a subject having a disease or disorder that would benefit from reduction in expression of a PAX5 gene, the method comprising administering to the subject a therapeutically effective amount of cells treated with the composition of any one of embodiments 102 to 104, thereby treating the subject.

113. The method of embodiment 112, wherein the disease or disorder comprises one or more of arthritis, atherosclerosis, breast cancer, cardiovascular disease, cataracts, chronic obstructive pulmonary disease, colorectal cancer, hypertension, osteoporosis, periodontitis, type 2 diabetes, immune dysfunction, Alzheimer's disease, leukemia, lymphoma, multiple sclerosis, Crohn's disease, HIV, influenza, pneumonia, lung cancer, melanoma, stroke, Parkinson's disease, and multiple drug resistant Staphylococcus aureus (MRSA).

114. A method for reducing the expression of a PPM1F gene, comprising exposing a cell to a composition comprising at least one isolated microRNA, wherein the at least one microRNA is at least 80% to 100% identical to SEQ ID NO:15.

115. The method of embodiment 114, wherein the composition further comprises a microRNA that is at least 80% to 100% identical to SEQ ID NO:16.

116. The method of embodiment 114 or 115, wherein the composition further comprises a microRNA that is at least 80% to 100% identical to SEQ ID NO:17.

117. The method of any one of embodiments 114 to 116, wherein the composition further comprises a microRNA that is at least 80% to 100% identical to SEQ ID NO:18.

118. The method of any one of embodiments 114 to 117, wherein the composition further comprises a microRNA that is at least 80% to 100% identical to SEQ ID NO:19.

119. The method of any one of embodiments 114 to 118, wherein the composition further comprises a microRNA that is at least 80% to 100% identical to SEQ ID NO:20.

120. A composition for reducing expression of a PPM1F gene comprising an acceptable carrier and at least one isolated microRNA that is at least 80% to 100% identical to SEQ ID NO:15.

121. The composition of embodiment 120, wherein the composition further comprises a microRNA that is at least 80% to 100% identical to SEQ ID NO:16.

122. The composition of embodiment 120 or 121, wherein the composition further comprises a microRNA that is at least 80% to 100% identical to SEQ ID NO:17.

123. The composition of any one of embodiments 120 to 122, wherein the composition further comprises a microRNA that is at least 80% to 100% identical to SEQ ID NO:18.

124. The composition of any one of embodiments 120 to 123, wherein the composition further comprises a microRNA that is at least 80% to 100% identical to SEQ ID NO:19.

125. The composition of any one of embodiments 120 to 124, wherein the composition further comprises a microRNA that is at least 80% to 100% identical to SEQ ID NO:20.

126. A method of reducing expression of a PPM1F gene in a cell, the method comprising: contacting the cell with the composition of any one of embodiments 120 to 125; and maintaining the cell for a time sufficient to obtain inhibition of a PPM1F gene, thereby reducing expression of the PPM1F gene in the cell.

127. The method of embodiment 126, wherein the cell is isolated from a subject.

128. The method of embodiment 127, wherein the subject is a human.

129. The method of any one of embodiments 126 to 128, wherein the PPM1F expression is reduced by at least about 30%, 40%, 50%, 60%, 70%, 80%, 90%, 95%, 98%, 99%, or 99.9%.

130. A method of treating a subject having a disease or disorder that would benefit from reduction in expression of a PPM1F gene, the method comprising administering to the subject a therapeutically effective amount of the composition of any one of embodiments 120 to 125, thereby treating the subject.

131. The method of embodiment 130, wherein the disease or disorder comprises one or more of arthritis, atherosclerosis, breast cancer, cardiovascular disease, cataracts, chronic obstructive pulmonary disease, colorectal cancer, hypertension, osteoporosis, periodontitis, type 2 diabetes, immune dysfunction, Alzheimer's disease, leukemia, lymphoma, multiple sclerosis, Crohn's disease, HIV, influenza, pneumonia, lung cancer, melanoma, stroke, Parkinson's disease, and multiple drug resistant Staphylococcus aureus (MRSA).

132. A method of treating a subject having a disease or disorder that would benefit from reduction in expression of a PAX5 gene and a reduction in expression of the PPM1F gene, the method comprising administering to the subject a therapeutically effective amount of the composition of any one of embodiments 102 to 107 and/or of any one of embodiments 120 to 125, thereby treating the subject.

133. The method of embodiment 132, wherein the subject is suffering from one or more of arthritis, atherosclerosis, breast cancer, cardiovascular disease, cataracts, chronic obstructive pulmonary disease, colorectal cancer, hypertension, osteoporosis, periodontitis, type 2 diabetes, immune dysfunction, Alzheimer's disease, leukemia, lymphoma, multiple sclerosis, Crohn's disease, HIV, influenza, pneumonia, lung cancer, melanoma, stroke, Parkinson's disease, and multiple drug resistant Staphylococcus aureus (MRSA).

134. A method for preparing a target cell comprising: obtaining cells from a subject to provide at least one subject cell; exposing the at least one subject cell to miRNA including one or more SEQ ID NOS:10-12 to provide at least one target cell.

135. The method of embodiment 134, wherein the at least one target cell is member of a population of cells comprising equal to or at least about 100, 1000, or 10,000 cells.

136. A method for treating or preventing cellular dysfunction in a patient, comprising administering to a patient in need thereof a therapeutically effective dose of the target cell of embodiment 134 or 135.

137. The method of embodiment 136, wherein the cellular dysfunction is an age related dysfunction.

138. The method of embodiment 136 or 137, wherein the cellular dysfunction one or more of arthritis, atherosclerosis, breast cancer, cardiovascular disease, cataracts, chronic obstructive pulmonary disease, colorectal cancer, hypertension, osteoporosis, periodontitis, type 2 diabetes, immune dysfunction, Alzheimer's disease, leukemia, lymphoma, multiple sclerosis, Crohn's disease, HIV, influenza, pneumonia, lung cancer, melanoma, stroke, Parkinson's disease, and multiple drug resistant Staphylococcus aureus (MRSA).

139. An miRNA for reducing the expression of PAX5 gene, wherein the miRNA comprises 4 to 50 contiguous nucleotides having a polynucleotide sequence that is at least 80% to 100% complementary to a region of SEQ ID NO:1.

140. An miRNA for reducing the expression of a PPM1F, wherein the miRNA comprises 4 to 50 contiguous nucleotides having a polynucleotide sequence that is at least 80% to 100% complementary to a region of SEQ ID NO:5.

141. A method for treating or preventing age related dysfunction, comprising administering to a patient in need thereof a therapeutically effective dose of one or more polycyclic aromatic compounds that antagonize or reduce the expression of PAX5 and/or PPM1F.

142. The method of embodiment 141, wherein the polycyclic compound is of formula I:

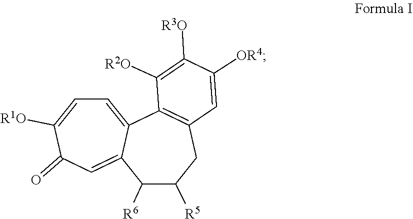

wherein

each of R₁, R₂, R₃, R₄, R₅, and R₆ is independently selected from —H, hydroxyl, halogen, C₁ to C₆ alkyl optionally substituted with halogen or hydroxy, optionally substituted C₁ to C₆ alkenyl, optionally substituted C₁ to C₆ alkynyl, optionally substituted C₁ to C₆ alkoxy, optionally substituted C₁ to C₆ haloalkyl, optionally substituted C₁ to C₆ haloalkoxy, mono-substituted amine(C₁ to C₆ alkyl optionally substituted), a di-substituted amine(C₁ to C₆ alkyl optionally substituted), a diamino-group, and an optionally substituted polyether—having 1 to 6 repeat units.

143. The method of embodiment 142, wherein each of R₁, R₂, R₃, R₄, R₅, and R₆ is independently selected from —H, hydroxyl, halogen, C₁ to C₆ alkyl optionally substituted with halogen or hydroxy, and a —(OR_{B}—)ₒOH, where R_{B} is an optionally substituted C₁ to C₆ alkyl.

144. The method of embodiment 141, wherein the polycyclic compound is of formula II:

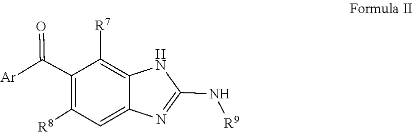

wherein

each of R₇, R₈, and R₉ is independently selected from —H, hydroxyl, halogen, C₁ to C₆ alkyl optionally substituted with halogen or hydroxy, optionally substituted C₁ to C₆ alkenyl, optionally substituted C₁ to C₆ alkynyl, optionally substituted C₁ to C₆ alkoxy, optionally substituted C₁ to C₆ haloalkyl, optionally substituted C₁ to C₆ haloalkoxy, mono-substituted amine(C₁ to C₆ alkyl optionally substituted), a di-substituted amine(C₁ to C₆ alkyl optionally substituted), a diamino-group, and an optionally substituted polyether—having 1 to 6 repeat units.

145. The method of embodiment 144, wherein each of R₇, R₈, and R₉ is independently selected from —H, hydroxyl, halogen, C₁ to C₆ alkyl optionally substituted with halogen or hydroxy, and a —(OR_{B}—)ₒOH, where R_{B} is an optionally substituted C₁ to C₆ alkyl.

146. The method of embodiment 141, wherein the polycyclic compound is of formula III:

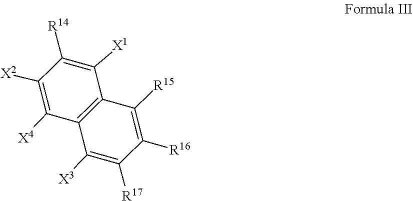

wherein

each of X₁, X₂, X₃, X₄ is independently selected from —H, hydroxyl, halogen, —NH₂, optionally substituted —SO₂OR₁₈;

each of R₁₄, R₁₅, R₁₆, R₁₇, and R₁₈ is independently selected from —H, hydroxyl, halogen, —NH₂, C₁ to C₆ alkyl optionally substituted with halogen or hydroxy, optionally substituted C₁ to C₆ alkenyl, optionally substituted C₁ to C₆ alkynyl, optionally substituted C₁ to C₆ alkoxy, optionally substituted C₁ to C₆ haloalkyl, optionally substituted C₁ to C₆ haloalkoxy, mono-substituted amine(C₁ to C₆ alkyl optionally substituted), a di-substituted amine(C₁ to C₆ alkyl optionally substituted), a diamino-group, and an optionally substituted polyether—having 1 to 6 repeat units.

147. The method of embodiment 146, wherein R₁₄, R₁₅, R₁₆, R₁₇, and R₁₈ are independently selected from —H, hydroxyl, halogen, C₁ to C₆ alkyl optionally substituted with halogen or hydroxy, and a —(OR_{B}—)ₒOH, where R_{B} is an optionally substituted C₁ to C₆ alkyl.

148. The method of embodiment 146, wherein the compound of formula III is represented by the following structure:

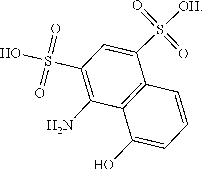 [SMILES: Nc1c(S(=O)(=O)O)cc(S(=O)(=O)O)c2cccc(O)c12]

149. The method of embodiment 141, wherein the polycyclic compound is selected from the group consisting of:

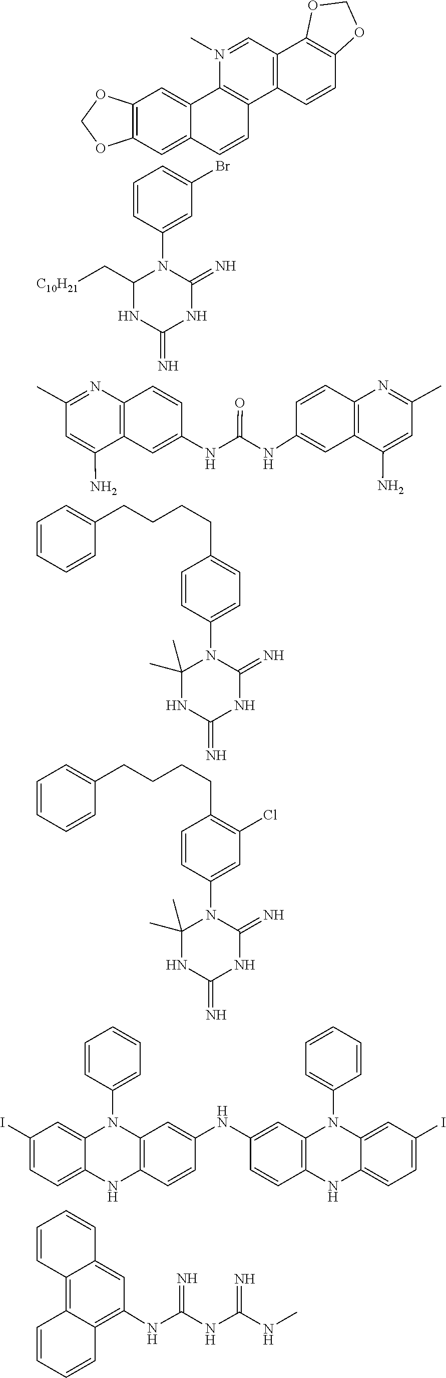

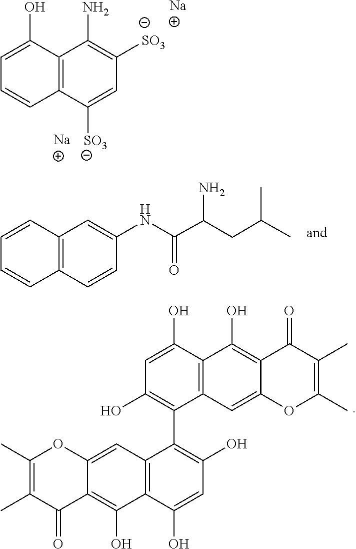

150. The method of any one of embodiments 141 to 149, wherein the polycyclic compound is provided as a pharmaceutically acceptable salt.

151. A pharmaceutical composition comprising a polycyclic aromatic compound as recited in any one of embodiments 141 to 150 and a pharmaceutically acceptable carrier.

152. A method of preparing at least one cell, the method comprising: providing at least one donor cell from a donor; providing at least one subject cell from a subject; providing at least one patient cell from a patient; exposing the subject cell to the donor cell to provide at least one intermediate cell; and exposing the patient cell to the intermediate cell to provide a target cell.

153. The method of embodiment 152, wherein exposing the subject cell to the donor cell comprises co-incubating the subject cell and the donor cell.

154. The method of embodiment 152 or 153, wherein exposing the intermediate cell to the patient cell comprises co-incubating the intermediate cell and the patient cell.

155. The method of any one of embodiments 152 to 154, wherein the subject cell is exposed to the donor cell for a time sufficient for cellular material from the donor cell to interact with the subject cell, thereby providing the intermediate cell.

156. The method of any one of embodiments 152 to 155, wherein the patient cell is exposed to the intermediate cell for a time sufficient for cellular material from the intermediate cell to interact with the patient cell, thereby providing the target cell.

157. The method of any one of embodiments 152 to 156, wherein each of the subject and the patient is older than the donor.

158. The method of any one of embodiments 152 to 157, wherein the donor cell is a cell mobilized from blood from the donor.

159. The method of any one of embodiments 152 to 158, wherein the subject cell is a cell mobilized from blood from the subject.

160. The method of any one of embodiments 152 to 159, wherein the patient cell is a cell that is mobilized from blood from the patient.

161. The method of any one of embodiments 158 to 160, further comprising administering a mobilizing agent to one or more of the donor, the subject, and the patient, wherein the mobilizing agent is an organic molecule, synthetic or naturally derived, or a polypeptide, such as a growth factor or colony-stimulating factor or an active fragment or mimic thereof, a nucleic acid, a carbohydrate, an antibody, or another agent that acts to enhance migration of stem cells from bone marrow to peripheral blood.

162. The method of any one of embodiments 152 to 161, wherein one or more of the donor cell, the subject cell, and/or the patient cell are harvested directly from the bone marrow of the donor, the subject, and/or the patient.

163. The method of any one of embodiments 152 to 162, wherein the subject is the patient.

164. The method of any one of embodiments 152 to 163, wherein the donor is the patient at an earlier age.

165. The method of any one of embodiments 152 to 164, wherein the target cell is provided in a formulation or culture suitable for administration to the patient to provide a therapeutic effect to the patient.

166. The method of any one of embodiments 152 to 165, wherein the at least one donor cell is member of a population of cells comprising equal to or at least about 10,000, 1,000,000, 10,000,000 cells.

167. The method of any one of embodiments 152 to 166, wherein the at least one subject cell is member of a population of cells comprising equal to or at least about 10,000, 1,000,000, 10,000,000 cells.

168. The method of any one of embodiments 152 to 167, wherein the at least one intermediate cell is member of a population of cells comprising equal to or at least about 10,000, 1,000,000, 10,000,000 cells.

169. The method of any one of embodiments 152 to 168, wherein the at least one target cell is member of a population of cells comprising equal to or at least about 10,000, 1,000,000, 10,000,000 cells.

170. The method of any one of embodiments 166 to 169, wherein one or more of the population of cells comprising the at least one donor cell, the population of cells comprising the at least one subject cell, the population of cells comprising the at least one intermediate cell, and/or the population of cells comprising the at least one target cell comprises one or more non-hematopoietic cells, mesenchymal stem cells, endothelial progenitor cells, hematopoietic stem cells, primitive hematopoietic stem cells, hematopoietic progenitor cells, differentiated hematopoietic cells, T-lymphocytes, natural killer cells, or combinations thereof.

171. A target cell made by the method any one of embodiments 152 to 170.

172. The target cell of embodiment 171, wherein the target cell is non-senescent or has decreased senescent behavior, has increased innate immune function, increased telomere length, lower replicative stress relative to the patient cell, increased stem cell clonogenicity, increased cytotoxic function, increased mitogen- and antigen-induced lymphocyte proliferation and activation, decreased myeloid to lymphoid ratio, increased CD4 to CD8 T lymphocyte ratio, decreased expression of senescence-associated secretory proteins, and/or decreased expression of senescence- and aging-related genes.

173. A composition comprising a population of cells comprising the target cell of any one of embodiments 152 to 172.

174. The composition of embodiment 173, wherein the composition comprises a pharmaceutically acceptable carrier.

175. The composition of embodiment 174, wherein the pharmaceutically acceptable carrier comprises one or more of an aqueous solution, cell culture media, or an aqueous buffered solution.

176. The composition of embodiment 174, wherein the pharmaceutically acceptable carrier comprises an aqueous solution of sodium chloride.

177. The composition of embodiment 176, wherein the pharmaceutically acceptable carrier further comprises human serum albumin.

178. The composition of embodiment 177, wherein the sodium chloride is present at about 0.9% by weight and/or wherein the human serum albumin is present at about 0.5% by weight.

179. A method for treating or preventing age-related dysfunction, comprising administering the target cell of any one of embodiments 152 to 171 or the composition of any one of embodiments 173 to 178 to the patient.

180. The method of embodiment 179, wherein the dysfunction is age-related dysfunction.

181. The method of embodiment 179 or 180, wherein the dysfunction includes one or more of arthritis, atherosclerosis, breast cancer, cardiovascular disease, cataracts, chronic obstructive pulmonary disease, colorectal cancer, hypertension, osteoporosis, periodontitis, type 2 diabetes, immune dysfunction, and Alzheimer's disease, leukemia, lymphoma, multiple sclerosis, Crohn's disease, HIV, influenza, pneumonia, lung cancer, melanoma, stroke, Parkinson's disease, multiple drug resistant Staphylococcus aureus (MRSA).

182. A method of reducing expression of a paired box 5 (PAX5) gene or reducing expression of a protein phosphatase 1F enzyme (PPM1F) gene in a cell, the method comprising: contacting a cell with one or more interfering RNAs (RNAi(s)) wherein the RNAi(s) comprises: one or more sequences comprising 4 to 50 contiguous nucleotides of a polynucleotide sequence that is at least 80% to 100% complementary to a region of SEQ ID NO:1, one or more sequences comprising a polynucleotide sequence that is at least 80% to 100% complementary to a region of SEQ ID NO:5, and/or one or more sequences comprising a polynucleotide sequence that is at least 80% to 100% identical to one or more of SEQ ID Nos: 9-20; and maintaining the cell for a time sufficient to obtain inhibition of the PAX5 gene or the PPM1F gene, thereby reducing expression of a PAX5 gene or a PPM1F gene in that cell to provide a target cell.

183. The method of embodiment 182, wherein the one or more RNAi(s) comprises at least one small interfering RNA (siRNA).

184. The method of embodiment 182 or 183, wherein the one or more RNAi(s) comprises at least one microRNA (miRNA).

185. The method of any one of embodiments 182 to 184, wherein the one or more RNAi(s) comprises at least one short hairpin RNA (shRNA).

186. The method of any one of embodiments 182 to 185, wherein the one or more RNAi(s) comprises SEQ ID NO:9.

187. The method of any one of embodiments 182 to 186, wherein the one or more RNAi(s) comprises SEQ ID NO:10.

188. The method of any one of embodiments 182 to 187, wherein the one or more RNAi(s) comprises SEQ ID NO:11.

189. The method of any one of embodiments 182 to 188, wherein the one or more RNAi(s) comprises SEQ ID NO:12.

190. The method of any one of embodiments 182 to 189, wherein the one or more RNAi(s) comprises SEQ ID NO:13.

191. The method of any one of embodiments 182 to 190, wherein the one or more RNAi(s) comprises SEQ ID NO:14.

192. The method of any one of embodiments 182 to 191, wherein the one or more RNAi(s) comprises SEQ ID NO:15.

193. The method of any one of embodiments 182 to 192, wherein the one or more RNAi(s) comprises SEQ ID NO:16.

194. The method of any one of embodiments 182 to 193, wherein the one or more RNAi(s) comprises SEQ ID NO:17.

195. The method of any one of embodiments 182 to 194, wherein the one or more RNAi(s) comprises SEQ ID NO:18.

196. The method of any one of embodiments 182 to 195, wherein the one or more RNAi(s) comprises SEQ ID NO:19.

197. The method of any one of embodiments 182 to 197, wherein the one or more RNAi(s) comprises SEQ ID NO:20.

198. The method of any one of embodiments 182 to 197, wherein the cell is a human cell.

199. The method of any one of embodiments 182 to 198, wherein the PAX5 expression is reduced by at least about 70% or wherein the PPM1F expression by at least about 70%.

200. The method of any one of embodiments 182 to 199, wherein the cell is contacted with the one or more RNAi(s) for a period of equal to or at least about 16 hours.

201. The method of any one of embodiments 182 to 200, wherein the target cell is non-senescent and/or has decreased senescent behavior, has increased innate immune function, increased telomere length, lower replicative stress relative to the patient cell, increased stem cell clonogenicity; increased cytotoxic function, increased mitogen- and/or antigen-induced lymphocyte proliferation and/or activation, decreased myeloid to lymphoid ratio, increased CD4 to CD8 T lymphocyte ratio, decreased expression of senescence-associated secretory proteins, and/or decreased expression of senescence- and/or aging-related genes.

202. A composition comprising: a pharmaceutically acceptable excipient; and one or more interfering RNAs (RNAi(s)); wherein the RNAi(s) comprises one or more sequences comprising: 4 to 50 contiguous nucleotides with a polynucleotide sequence that is at least 80% to 100% complementary to a region of SEQ ID NO:1, one or more sequences comprising a polynucleotide sequence that is at least 80% to 100% complementary to a region of SEQ ID NO:5, and/or one or more sequences comprising a polynucleotide sequence that is at least 80% to 100% identical to one or more of SEQ ID Nos: 9-20; wherein the composition is configured to reduce an expression of a paired box 5 (PAX5) gene or an expression of a protein phosphatase 1F enzyme (PPM1F) gene in a cell.

203. A method for treating or preventing age related dysfunction, comprising administering to a patient in need thereof a therapeutically effective dose of one or more polycyclic aromatic compounds that: antagonize a PAX5 protein and/or antagonize PP1F protein or reduce an expression of a PAX5 gene and/or a PPM1F gene.

204. The method of embodiment 203, wherein the polycyclic compound is of formula I:

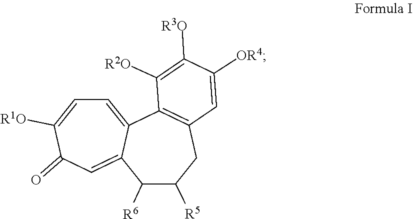

wherein each of R₁, R₂, R₃, R₄, R₅, and R₆ is independently selected from —H, hydroxyl, halogen, C₁ to C₆ alkyl optionally substituted with halogen or hydroxy, optionally substituted C₁ to C₆ alkenyl, optionally substituted C₁ to C₆ alkynyl, optionally substituted C₁ to C₆ alkoxy, optionally substituted C₁ to C₆ haloalkyl, optionally substituted C₁ to C₆ haloalkoxy, mono-substituted amine(C₁ to C₆ alkyl optionally substituted), a di-substituted amine(C₁ to C₆ alkyl optionally substituted), a diamino-group, and an optionally substituted polyether—having 1 to 6 repeat units.

205. The method of embodiment 203, wherein the polycyclic compound is of formula II:

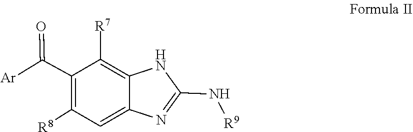

wherein each of R₇, R₈, and R₉ is independently selected from —H, hydroxyl, halogen, C₁ to C₆ alkyl optionally substituted with halogen or hydroxy, optionally substituted C₁ to C₆ alkenyl, optionally substituted C₁ to C₆ alkynyl, optionally substituted C₁ to C₆ alkoxy, optionally substituted C₁ to C₆ haloalkyl, optionally substituted C₁ to C₆ haloalkoxy, mono-substituted amine(C₁ to C₆ alkyl optionally substituted), a di-substituted amine(C₁ to C₆ alkyl optionally substituted), a diamino-group, and an optionally substituted polyether—having 1 to 6 repeat units.

206. The method of embodiment 203, wherein the polycyclic compound is of formula III:

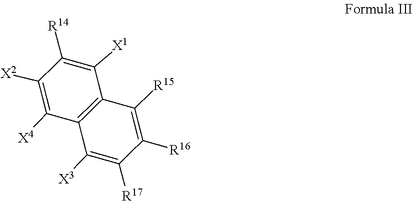

wherein each of X₁, X₂, X₃, X₄ is independently selected from —H, hydroxyl, halogen, —NH₂, optionally substituted —SO₂OR₁₈; each of R₁₄, R₁₅, R₁₆, R₁₇, and R₁₈ is independently selected from —H, hydroxyl, halogen, —NH₂, C₁ to C₆ alkyl optionally substituted with halogen or hydroxy, optionally substituted C₁ to C₆ alkenyl, optionally substituted C₁ to C₆ alkynyl, optionally substituted C₁ to C₆ alkoxy, optionally substituted C₁ to C₆ haloalkyl, optionally substituted C₁ to C₆ haloalkoxy, mono-substituted amine(C₁ to C₆ alkyl optionally substituted), a di-substituted amine(C₁ to C₆ alkyl optionally substituted), a diamino-group, and an optionally substituted polyether—having 1 to 6 repeat units.

207. The method of embodiment 203, wherein the polycyclic compound is selected from the group consisting of:

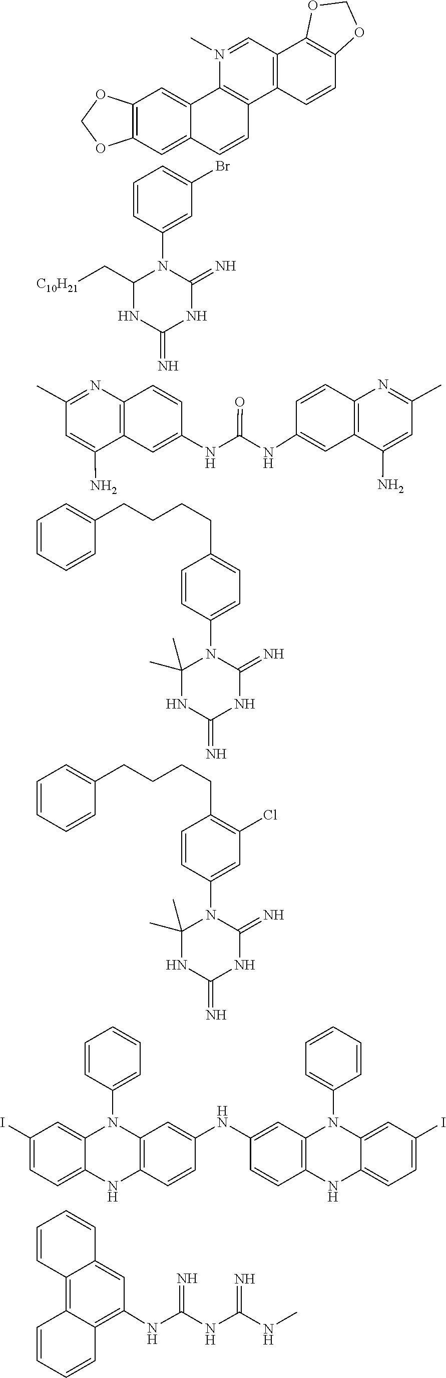

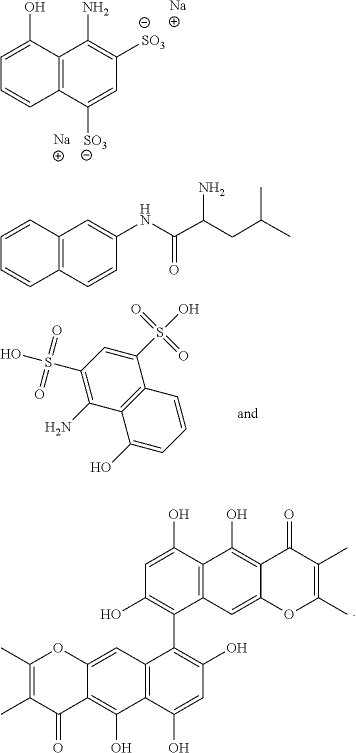

208. A method of preparing at least one cell, the method comprising: providing at least one donor cell from a donor; providing at least one subject cell from a subject; providing at least one patient cell from a patient; exposing the subject cell to the donor cell to provide at least one intermediate cell; and exposing the patient cell to the intermediate cell to provide a target cell.

209. The method of embodiment 208, wherein exposing the subject cell to the donor cell comprises co-incubating the subject cell and the donor cell; and wherein exposing the intermediate cell to the patient cell comprises co-incubating the intermediate cell and the patient cell.

210. The method of embodiment 208 or 209, wherein the subject cell is exposed to the donor cell for a time sufficient for cellular material from the donor cell to interact with the subject cell, thereby providing the intermediate cell; wherein the patient cell is exposed to the intermediate cell for a time sufficient for cellular material from the intermediate cell to interact with the patient cell, thereby providing the target cell.

211. The method of any one of embodiments 207 to 210, wherein the donor is the patient at an earlier age and the donor is the subject at an earlier age.

### Additional Enumerated Embodiments

The following provide additional exemplary illustrative enumerated embodiments.

1. A method for preparing at least one target cell for use in treating a patient with cellular dysfunctional or an age-related disorder, the method comprising:

providing at least one donor cell from a donor;

providing at least one patient cell from a patient;

exposing the patient cell to the donor cell in an environment that is substantially-free of animal-based factors to provide at least one target cell.

2. The method of embodiment 1, wherein the donor is younger than the patient.

3. The method of embodiment 1 or 2, wherein the donor cell is exposed to the subject cell in a manner that prevents the donor cell and the subject cell from becoming mixed.

4. The method of any one of embodiments 1 to 3, wherein the donor cell is provided as a cryogenically frozen donor cell that is thawed prior to exposure to the subject cell.

5. The method of any one of embodiments 1 to 4, wherein the subject cell is provided as a cryogenically frozen subject cell that is thawed prior to exposure to the donor cell.

6. A method for preparing at least one target cell for use in treating a patient with cellular dysfunctional or an age-related disorder, the method comprising:

providing at least one patient cell from the patient;

contacting the patient cell with one or more interfering RNA(s) (RNAi(s)) selected from SEQ ID NOs:9-20 and/or one or more small molecule drugs that inhibit PAX5 and/or PPM1F in an environment that is substantially-free of animal-based factors to provide a target cell.

7. The method of embodiment 6, further comprising exposing the patient to the target cell thereby treating the patient.

8. The method of embodiment 6 or 7, wherein the patient cell is provided as a cryogenically frozen patient cell that is thawed prior to contact with the interfering RNA(s) and/or small molecule drugs.

9. The method of any one of embodiments 6 to 8, wherein the target cell is provided as a cryogenically frozen target cell that is thawed prior to administration to the patient.

10. A method for preparing at least one target cell for use in treating a patient with cellular dysfunctional or an age-related disorder, the method comprising:

providing at least one patient cell from the patient;

contacting the patient cell with one or more interfering RNA(s) (RNAi(s)) having at least 80% identity to one or more of SEQ ID NOs:9-20 in an environment that is substantially-free of animal-based factors to provide a target cell.

11. The method of embodiment 10, further comprising exposing the patient to the target cell thereby treating the patient.

12. The method of embodiment 10 or 11, wherein the patient cell is provided as a cryogenically frozen patient cell that is thawed prior to contact with the interfering RNA(s).

13. The method of any one of embodiments 10 to 12, wherein the target cell is provided as a cryogenically frozen target cell that is thawed prior to administration to the patient.

14. A target cell made by the method of any one of embodiments 1 to 13.

15. A pharmaceutical composition comprising a target cell made by the method of any one of embodiments 1 to 13.

16. A method of treating a patient with cellular dysfunctional or an age-related disorder, the method comprising:

providing at least one donor cell from a donor;

providing at least one patient cell from a patient;

exposing the patient cell to the donor cell in an environment that is substantially-free of animal-based factors to provide at least one target cell; and

exposing the patient to the target cell thereby treating the patient.

17. The method of embodiment 16, wherein the donor is younger than the patient and/or wherein the donor is that patient at a younger age.

18. The method of embodiment 16 or 17, wherein the donor cell is exposed to the subject cell in a manner that prevents the donor cell and the subject cell from becoming mixed.

19. The method of any one of embodiments 16 to 18, wherein the donor cell is provided as a cryogenically frozen donor cell that is thawed prior to exposure to the subject cell.

20. The method of any one of embodiments 16 to 19, wherein the subject cell is provided as a cryogenically frozen subject cell that is thawed prior to exposure to the donor cell.

21. A method for treating a patient with cellular dysfunctional or an age-related disorder, the method comprising:

providing at least one patient cell from the patient;

contacting the patient cell with one or more interfering RNA(s) (RNAi(s)) selected from SEQ ID NOs:9-20 and/or one or more small molecule drugs that inhibit PAX5 and/or PPM1F to provide a target cell; and

exposing the patient to the target cell thereby treating the patient.

22. A method for treating a patient with cellular dysfunctional or an age-related disorder, the method comprising:

providing at least one patient cell from the patient;

contacting the patient cell with one or more interfering RNA(s) (RNAi(s)) having at least 80% identity to one or more of SEQ ID NOs:9-20 to provide a target cell; and

exposing the patient to the target cell thereby treating the patient.

23. The method of embodiment 21 or 22, wherein the patient cell is provided as a cryogenically frozen patient cell that is thawed prior to contact with the interfering RNA(s) and/or small molecule drugs.

24. The method of any one of embodiments 21 to 23, wherein the target cell is provided as a cryogenically frozen target cell that is thawed prior to administration to the patient.

25. The method of any one of embodiments 6 to 13 or 21 to 24, further comprising exposing the patient to the one or more small molecule drugs.

26. A method for treating a patient with cellular dysfunctional or an age-related disorder, the method comprising:

providing at least one donor cell from a donor;

providing at least one subject cell from a subject;

providing at least one patient cell from a patient;

exposing the subject cell to the donor cell in an environment that is substantially-free of animal-based factors to provide at least one intermediate cell;

exposing the patient cell to the intermediate cell in an environment that is substantially-free of animal-based factors to provide at least one target cell; and

exposing the patient to the target cell thereby treating the patient.

27. The method of embodiment 26, further comprising at least contacting the patient cell with one or more RNAi(s) selected from SEQ ID NOs:9-20 and/or one or more small molecule drugs that inhibit PAX5 and/or PPM1F in an environment that is substantially-free of animal-based factors to provide a target cell.

28. The method of any one of embodiments 1 to 27, wherein the patient and donor are related by consanguinity.

29. The method of any one of embodiments 1 to 28, wherein the one or more small molecule drugs is a polycyclic aromatic compound that antagonize a PAX5 protein and/or PP1F protein or reduce the expression of a PAX5 gene and/or a PPM1F gene.

30. The method of embodiment 29, wherein the polycyclic aromatic compound is of formula I:

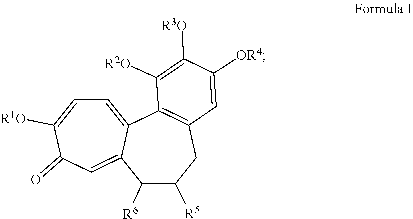

wherein each of R₁, R₂, R₃, R₄, R₅, and R₆ is independently selected from —H, hydroxyl, halogen, C₁ to C₆ alkyl optionally substituted with halogen or hydroxy, optionally substituted C₁ to C₆ alkenyl, optionally substituted C₁ to C₆ alkynyl, optionally substituted C₁ to C₆ alkoxy, optionally substituted C₁ to C₆ haloalkyl, optionally substituted C₁ to C₆ haloalkoxy, mono-substituted amine(C₁ to C₆ alkyl optionally substituted), a di-substituted amine(C₁ to C₆ alkyl optionally substituted), a diamino-group, and an optionally substituted polyether—having 1 to 6 repeat units.

31. The method of embodiment 30, wherein each of R₁, R₂, R₃, R₄, R₅, and R₆ is independently selected from —H, hydroxyl, halogen, C₁ to C₆ alkyl optionally substituted with halogen or hydroxy, and a —(OR_{B}—)ₒOH, where R_{B} is an optionally substituted C₁ to C₆ alkyl.

32. The method of embodiment 29, wherein the polycyclic compound is of formula II:

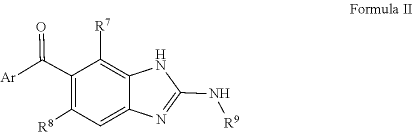

wherein each of R₇, R₈, and R₉ is independently selected from —H, hydroxyl, halogen, C₁ to C₆ alkyl optionally substituted with halogen or hydroxy, optionally substituted C₁ to C₆ alkenyl, optionally substituted C₁ to C₆ alkynyl, optionally substituted C₁ to C₆ alkoxy, optionally substituted C₁ to C₆ haloalkyl, optionally substituted C₁ to C₆ haloalkoxy, mono-substituted amine(C₁ to C₆ alkyl optionally substituted), a di-substituted amine(C₁ to C₆ alkyl optionally substituted), a diamino-group, and an optionally substituted polyether—having 1 to 6 repeat units.

33. The method of embodiment 32, wherein each of R₇, R₈, and R₉ is independently selected from —H, hydroxyl, halogen, C₁ to C₆ alkyl optionally substituted with halogen or hydroxy, and a —(OR_{B}—)ₒOH, where R_{B} is an optionally substituted C₁ to C₆ alkyl.

34. The method of embodiment 29, wherein the polycyclic compound is of formula III:

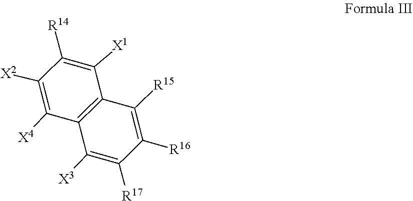

wherein each of X₁, X₂, X₃, X₄ is independently selected from —H, hydroxyl, halogen, —NH₂, optionally substituted —SO₂OR₁₈; and

wherein each of R₁₄, R₁₅, R₁₆, R₁₇, and R₁₈ is independently selected from —H, hydroxyl, halogen, —NH₂, C₁ to C₆ alkyl optionally substituted with halogen or hydroxy, optionally substituted C₁ to C₆ alkenyl, optionally substituted C₁ to C₆ alkynyl, optionally substituted C₁ to C₆ alkoxy, optionally substituted C₁ to C₆ haloalkyl, optionally substituted C₁ to C₆ haloalkoxy, mono-substituted amine(C₁ to C₆ alkyl optionally substituted), a di-substituted amine(C₁ to C₆ alkyl optionally substituted), a diamino-group, and an optionally substituted polyether—having 1 to 6 repeat units.

35. The method of embodiment 34, wherein R₁₄, R₁₅, R₁₆, R₁₇, and R₁₈ are independently selected from —H, hydroxyl, halogen, C₁ to C₆ alkyl optionally substituted with halogen or hydroxy, and a —(OR_{B}—)ₒOH, where R_{B} is an optionally substituted C₁ to C₆ alkyl.

36. The method of embodiment 34, wherein the compound of formula III is represented by the following structure:

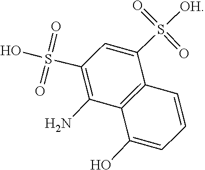 [SMILES: Nc1c(S(=O)(=O)O)cc(S(=O)(=O)O)c2cccc(O)c12]

37. The method of embodiment 29, wherein the polycyclic compound is selected from the group consisting of:

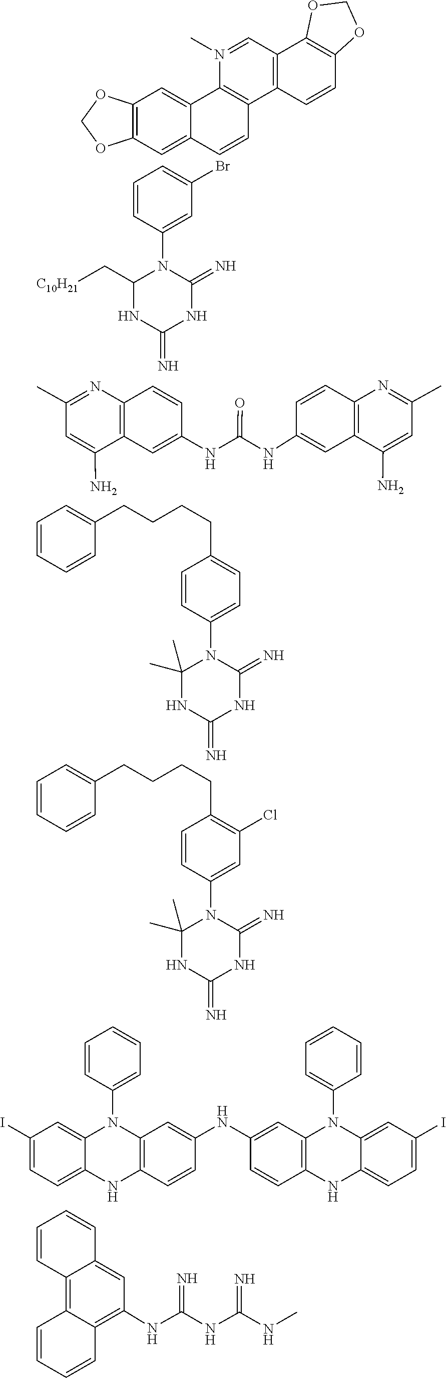

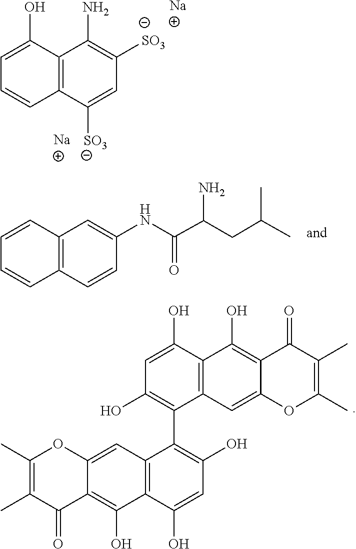

38. The method of any one of embodiments 29 to 37, wherein the polycyclic compound is provided as a pharmaceutically acceptable salt.

39. The method of any one of embodiments 1 to 13 or 16 to 38, or the cell of embodiment 14, or the pharmaceutical composition of embodiment 15, wherein the cellular dysfunctional or age-related disorder is cancer, breast cancer, colorectal cancer, liver cancer, kidney cancer, brain cancer, pancreatic cancer, lung cancer, stomach cancer, uterine cancer, ovarian cancer, prostate cancer, testicular cancer, thyroid cancer, carcionoma, myeloma, sarcoma, leukemia, lymphoma, melanoma, hematological malignancy, arthritis, atherosclerosis, cardiovascular disease, cataracts, chronic obstructive pulmonary disease, hypertension, osteoporosis, periodontitis, diabetes, Alzheimer's disease, stroke, Parkinson's disease, multiple sclerosis, Crohn's disease, HIV, influzena, pneumonia, or MRSA.

40. The method of any one of embodiments 1 to 13 or 16 to 38, or the cell of embodiment 14, or the pharmaceutical composition of embodiment 15, wherein the at least one patient cell comprises an immune cell, neutrophil, macrophage, natural killer cell, eosinophil, basophil, mast cell, dendritic cell, T cell or B cell or any combination thereof, and exposing the patient cell to the donor cell improves the immune activity of the patient cell.

41. The method of any one of embodiments 1 to 13 or 16 to 38, or the cell of embodiment 14, or the pharmaceutical composition of embodiment 15, further comprising administering G-CSF, filgrastim, lenograstim, or ancestim to the donor or patient.

42. The method of any one of embodiments 1 to 13 or 16 to 38, or the cell of embodiment 14, or the pharmaceutical composition of embodiment 15, wherein the donor and/or patient is a mammal.

43. The method of any one of embodiments 1 to 13 or 16 to 38, or the cell of embodiment 14, or the pharmaceutical composition of embodiment 15, wherein the donor and/or patient is a human.

44. A kit for collecting blood from a patient or a donor, the kit comprising:

liquid collection containers;

a laboratory directive; and

instructions for the blood drawing from the patient or the donor.

45. The kit of embodiment 44, further comprising an enclosing container configured to house other components of the kit.

46. The kit of embodiment 44 or 45, further comprising a shipping envelope.

47. The kit of embodiment 46, wherein the shipping envelope is prepaid.

48. The kit of embodiment 46 or 47, wherein the shipping envelope provides for overnight shipping.

49. The kit of any one of embodiments 44 to 48, further comprising a national lab directive.

50. The kit of embodiment 49, wherein the lab directive provides instructions for blood sample processing.

51. The kit of any one of embodiments 44 to 50, further comprising a lab requisition form.

52. The kit of embodiment 51, wherein the lab requisition form is for a national laboratory.

53. The kit of embodiment 51 or 52, wherein the lab requisition form is a Quest National Lab Requistion form.

54. The kit of any one of embodiments 44 to 53, further comprising a biohazard container.

55. The kit of embodiment 54, wherein the biohazard container is a bag.

56. The kit of any one of embodiments 44 to 55, wherein the laboratory directive comprises blood drawing instructions.

57. The kit of any one of embodiments 44 to 56, further comprising a packing material.

58. The kit of embodiment 57, wherein the packing material is bubble wrap.

59. The kit of any one of embodiments 44 to 57, further comprising a patient self-evaluation form.

60. The kit of embodiment 59, wherein the self-evaluation form is a quality of life form.

61. The kit of embodiment 59 or 60, wherein the self-evaluation form is a SF-36 quality of life survey.

62. The kit of any one of embodiments 44 to 61, wherein the liquid collection containers are configured to receive blood.

63. The kit of any one of embodiments 44 to 62, wherein the liquid collection containers are blood collection tubes or vials.

64. The kit of any one of embodiments 44 to 63, further comprising a diagnostic testing unit.

65. The kit of embodiment 64, wherein the diagnostic testing unit comprises a diagnostic testing kit comprising one or more of a myeloid leukemia panel, a myeloid/lymphoid ratio assay, a lymphocyte proliferative response assay, a natural killer cytotoxicity assay, a T helper cell/killer T cell ratio assay, and/or a complete blood count assay.

66. The kit of embodiment 65, wherein the lymphocyte proliferative response assay is mitogen-based and/or antigen-based.

67. The kit of any one of embodiments 64 to 66, wherein the diagnostic testing unit comprises biochemical and/or genetic biomarker assays.

68. The kit of embodiment 67, wherein the diagnostic testing unit comprises one or more of a senescence gene array, an aging gene array, and/or a senescence protein array.

69. The kit of embodiment 68, wherein the senescence gene array and/or aging gene array is configured to measure mononuclear cells in blood.

70. The kit of embodiment 68 or 69, wherein the senescence protein array is configured to measure blood plasma proteins.

71. The kit of any one of embodiments 44 to 70, further comprising instructions indicating that the diagnostic testing should be performed about every month.

72. The kit of any one of embodiments 44 to 71, further comprising instructions indicating that a physical examination of the patient should be performed about every 12 to 24 months.

73. The kit of any one of embodiments 44 to 79, wherein the patient instructions and/or the self-evaluation form indicates that it should be completed about every three months.

74. The kit of any one of embodiments 44 to 73, further comprising instructions indicating that a baseline physical examination and diagnostic testing should be performed prior to treatment.

75. A method for preparing at least one target cell for use in treating a patient with cellular dysfunctional or an age-related disorder, the method comprising:

providing at least one donor cell from a donor;

providing at least one patient cell from a patient;

exposing the patient cell to the donor cell in an environment that is substantially-free of animal-based factors to provide at least one target cell.

76. The method of embodiment 75, wherein the donor is younger than the patient.

77. The method of embodiment 75 or 76, wherein the donor cell is exposed to the subject cell in a manner that prevents the donor cell and the subject cell from becoming mixed.

78. The method of any one of embodiments 75 to 77, wherein the donor cell is provided as a cryogenically frozen donor cell that is thawed prior to exposure to the subject cell.

79. The method of any one of embodiments 75 to 78, wherein the subject cell is provided as a cryogenically frozen subject cell that is thawed prior to exposure to the donor cell.

80. A method for preparing at least one target cell for use in treating a patient with cellular dysfunctional or an age-related disorder, the method comprising:

providing at least one patient cell from the patient;

contacting the patient cell with one or more interfering RNA(s) (RNAi(s)) selected from SEQ ID NOs:9-20 and/or one or more small molecule drugs that inhibit PAX5 and/or PPM1F in an environment that is substantially-free of animal-based factors to provide to provide a target cell.

81. The method of embodiment 80, further comprising exposing the patient to the target cell thereby treating the patient.

82. The method of embodiment 80 or 81, wherein the patient cell is provided as a cryogenically frozen patient cell that is thawed prior to contact with the interfering RNA(s) and/or small molecule drugs.

83. The method of any one of embodiments 80 to 82, wherein the target cell is provided as a cryogenically frozen target cell that is thawed prior to administration to the patient.

84. A method for preparing at least one target cell for use in treating a patient with cellular dysfunctional or an age-related disorder, the method comprising:

providing at least one patient cell from the patient;

contacting the patient cell with one or more interfering RNA(s) (RNAi(s)) having at least 80% identity to one or more of SEQ ID NOs:9-20 in an environment that is substantially-free of animal-based factors to provide a target cell.

85. The method of embodiment 84, further comprising exposing the patient to the target cell thereby treating the patient.

86. The method of embodiment 84 or 85, wherein the patient cell is provided as a cryogenically frozen patient cell that is thawed prior to contact with the interfering RNA(s).

87. The method of any one of embodiments 84 to 86, wherein the target cell is provided as a cryogenically frozen target cell that is thawed prior to administration to the patient.

88. A target cell made by the method of any one of embodiments 65 to 87.

89. A pharmaceutical composition comprising a target cell made by the method of any one of embodiments 75 to 88.

90. A method of treating a patient with cellular dysfunctional or an age-related disorder, the method comprising:

providing at least one donor cell from a donor;

providing at least one patient cell from a patient;

exposing the patient cell to the donor cell in an environment that is substantially-free of animal-based factors to provide at least one target cell; and

exposing the patient to the target cell thereby treating the patient;

wherein at least one of the donor or patient cell is collected using a kit as recited in any one of embodiments 44 to 74.

91. A method of treating a patient with cellular dysfunctional or an age-related disorder, the method comprising:

providing at least one donor cell from a donor;

providing at least one patient cell from a patient;

exposing the patient cell to the donor cell in an environment that is substantially-free of animal-based factors to provide at least one target cell; and

exposing the patient to the target cell thereby treating the patient.

92. The method of embodiment 91, wherein the donor is younger than the patient and/or wherein the donor is that patient at a younger age.

93. The method of embodiment 91 or 92, wherein the donor cell is exposed to the subject cell in a manner that prevents the donor cell and the subject cell from becoming mixed.

94. The method of any one of embodiments 91 to 93, wherein the donor cell is provided as a cryogenically frozen donor cell that is thawed prior to exposure to the subject cell.

95. The method of any one of embodiments 91 to 94, wherein the subject cell is provided as a cryogenically frozen subject cell that is thawed prior to exposure to the donor cell.

96. A method for treating a patient with cellular dysfunctional or an age-related disorder, the method comprising:

providing at least one patient cell from the patient;

contacting the patient cell with one or more interfering RNA(s) (RNAi(s)) selected from SEQ ID NOs:9-20 and/or one or more small molecule drugs that inhibit PAX5 and/or PPM1F to provide a target cell; and

exposing the patient to the target cell thereby treating the patient.

97. A method for treating a patient with cellular dysfunctional or an age-related disorder, the method comprising:

providing at least one patient cell from the patient;

contacting the patient cell with one or more interfering RNA(s) (RNAi(s)) having at least 80% identity to one or more of SEQ ID NOs:9-20 to provide a target cell; and

exposing the patient to the target cell thereby treating the patient.

98. The method of embodiment 96 or 97, wherein the patient cell is provided as a cryogenically frozen patient cell that is thawed prior to contact with the interfering RNA(s) and/or small molecule drugs.

99. The method of any one of embodiments 96 to 98, wherein the target cell is provided as a cryogenically frozen target cell that is thawed prior to administration to the patient.

It should be understood that the embodiments provided herein, such as under the Enumerated Embodiments or Additional Enumerated Embodiments sections, can be combined with, modified by, or excluded from any other embodiments provided herein. For instance, a kit as recited in the Additional Enumerated Embodiments, may comprise a RNAi or small molecule of an Enumerated Embodiment. A kit as disclosed in the Additionaly Enumerated Embodiments may contain instructions on how to perform a method as disclosed in the Enumerated Embodiments. It should be further understood that any element of the embodiments provided herein can be added to, or excluded from any other embodiment provided herein. It should be further understood that any of the kit embodiments provided herein can be modified by any of the method embodiments or any element of any of the method embodiments. It should be further understood that any of the method embodiments provided herein can be modified by any of the kit embodiments or any element of any of the kit embodiments. For example, any embodiment provided herein can be modified to include any of the small molecule compounds, polycyclic compounds, interfering RNAs, miRNAs, siRNAs, shRNA, or any modification or pharmaceutically acceptable salt thereof provided in any other embodiment provided herein, including the kit embodiments or the method embodiments. For further illustration, any embodiment provided herein can be modified to include any of the small molecule compounds, polycyclic compounds, interfering RNAs, miRNAs, siRNAs, shRNA, or any modification or pharmaceutically acceptable salt thereof provided in any other embodiment provided herein, including the method embodiments or composition of matter embodiments.

### EXAMPLES

The following examples describe one or more process steps and methods that have been developed for the treatment of patients in need thereof. It has been discovered that, by using one or more of the following steps individually or in combination, increased cell yields and/or treatment efficacy can be achieved. FIG. 3A is a schematic overview of an embodiment of a process for preparing therapeutic target cells for use in a human patient as disclosed in one or more of the following examples. FIGS. 3B-3C provide additional schematics of the clinical process, including detailing logistics for shipping and transport of cells to various facilities and patient locations. FIG. 4 is a flow diagram showing an embodiment of a process for preparing therapeutic target cells for use in a human patient as disclosed in one or more of the following examples. In some embodiments, the operations in the examples and elsewhere herein can be carried out under strict aseptic conditions.

### Example 1

One or more of the following steps can be employed in the screening of patients and donors. As shown, the process may include steps for screening patients and donors (FIGS. 3.1 and 4.1), mobilization of the cells of the donor and patient (FIGS. 3A.2 and 4.2), collection of the cells (FIGS. 3A.3 and 4.2), the cooling of cells for transport (FIGS. 3A.4 and 4.3), the transport of cells to treatment facilities or long term storage (FIGS. 4.4-4.8) and storage of cells from donors and patients (FIGS. 3A.5 and 4.6).

### Donor Selection and Collection.

In some embodiments, collections are performed on aged and young individuals for stem cell mobilization and leukapheresis (FIGS. 3A.1-3A.3). In some embodiments, age restrictions are employed during the screening process (FIGS. 3A.1 and 4.1). In some embodiments, the age restrictions include one or more of the following: Aged donors, >59 y/o; Young donors, 18-29 y/o. Sex (No restrictions). Ethnicity (No restrictions).

### Non-Specific Inclusion/Exclusion Criteria for Donor Enrollment

Inclusion Criteria: Participants are screened and must meet all of the following criteria to be eligible to provide mobilized mononuclear cell samples. Normal pulse (without irregularities) and in the range of 50 to 100 beats per minute; Normal blood pressure. Participants will be further evaluated for inclusion by the PI under the following conditions: a. Systolic pressure of <100 or >160 mm Hg; Diastolic pressure of <60 or >90 mm Hg; Results of urinalysis and basic chemistry panel performed during prescreening within normal limits; WBC >4.1×103/μL; % mononuclear cells (monocytes and lymphocytes): 15-55%; Absolute lymphocyte count: >0.60×103/μL; Test negative for the following infectious disease markers: HIV, Hepatitis B and C, HTLV and syphilis.

Exclusion Criteria: Participants who meet any of the following criteria are not eligible to provide mobilized mononuclear cell samples: Pregnant or breastfeeding; Current bleeding disorder, or history of bleeding disorders; History of hemoglobinopathy (e.g. sickle cell disease, thalassemia); History of myelodysplastic disorder; Autoimmune disease; Temperature >99.5° C.; Hemoglobin <12.5 g/dL or Hematocrit <38%; Platelet count <150×103/μL; and Absolute neutrophil count <1500/μL.

In some embodiments, the inclusion and exclusion criteria include one or more of the factors provided in Table 4 below.

TABLE 4
Inclusion and Exclusion Criteria for
Study Donors (Young) and Patients (Aged)

| Donor | Aged | Young |
|---|---|---|
| Inclusion | ≥60 years old | 18-29 years old |
| Criteria | Healthy and feeling well | Healthy and feeling well |
| | BMI of 18.5-29 | Normal BMI (18.5-25) |
| | Weigh at least 140 lbs | Weigh at least 120 lbs |
| | Successful Leukopak | Vaccination record current |
| | donation | Successful Leukopak donation |
| | Meet protocol | Meet protocol specifications, |
| | specifications, | i.e. CBC lab test |
| | i.e. CBC lab test | At least 5 days/week of |
| | Vaccination record current | moderate to strenuous |
| | Have adequate peripheral | exercise (minimum of 30 min) |
| | veins for apheresis | Successful completion of |
| | Review and sign an | physical examination |
| | IRB-approved | Non-smoker |
| | procedure-specific consent | Between 5-15% body |
| | form prior to the collection | fat for men; 12.5-25% |
| | Fill out donor history | body fat for women |
| | questionnaire | Healthy eating habits/diet |
| | Non-smoker | with consumption of |
| | | fish 2× per week regularly |
| | | Obtains 6-8 hours of sleep |
| | | per night on a regular basis |
| | | Have adequate peripheral |
| | | veins for apheresis |
| | | Review and sign an IRB- |
| | | approved procedure-specific |
| | | consent form prior to |
| | | the collection |
| | | Fill out Donor |
| | | History Questionnaire |
| Exclusion | Current or recent | Current or recent (< 30 |
| Criteria | (< 30 days) illness | days) illness |
| | Underweight (< 18.5) | Abnormal BMI (underweight, |
| | or Obese (> 29) BMI | overweight, obese) |
| | Cannot be pregnant | Diet consisting of fast food |
| | Prior cancer diagnosis | more than once per week |
| | Previously mobilized | Moderate to heavy regular |
| | HIV, HPV, HBV or | alcohol consumption |
| | HCV positive test | Cannot be pregnant |
| | History of heart, lung, | Prior cancer diagnosis |
| | liver, kidney disease | Previously mobilized |
| | Blood or bleeding disorders | HIV, HPV, HBV or |
| | Neurologic disorders | HCV positive test |
| | Diabetes | History of heart, lung, |
| | Autoimmune disorders | liver, kidney disease |
| | | Blood or bleeding disorders |
| | | Neurologic disorders |
| | | Diabetes |
| | | Autoimmune disorders |

Additional inclusion and exclusion criteria can include one or more of the following: Inclusion Criteria: In some embodiments, participants must meet one or more of the following criteria to be eligible to provide mobilized mononuclear cell samples: Normal pulse (without irregularities) and in the range of 50 to 100 beats per minute; Normal blood pressure; a. Systolic pressure of <100 or >160 mm Hg; Diastolic pressure of <60 or >90 mm Hg; Results of urinalysis and basic chemistry panel performed during prescreening within normal limits; WBC>4.1×10³/μL; % mononuclear cells (monocytes and lymphocytes): 15-55%; Absolute lymphocyte count: >0.60×10³/μL; test negative for the following infectious disease markers: HIV, Hepatitis B and C, HTLV and syphilis. In some embodiments, participants who meet one or more of the following criteria (exclusion criteria) will not be eligible to provide mobilized mononuclear cell samples: Pregnant or breastfeeding; Current bleeding disorder, or history of bleeding disorders; History of hemoglobinopathy (e.g. sickle cell disease, thalassemia); History of myelodysplastic disorder; Autoimmune disease; Temperature >99.5° C.; Hemoglobin <12.5 g/dL or Hematocrit <38%; Platelet count <150×10³/μL; Absolute neutrophil count <1500/μL.

In some embodiments, study participants will have peripheral blood collected prior to mobilization for baseline CBC, immune cell phenotyping and stimulation response (see methodology). In some embodiments, the study is longitudinal, with efficacy determined by comparison of efficacy measures at 2, 6, 12 and 24 months post-treatment to baseline (pre-treatment).

### Methodology

### Stem Cell Mobilization and Leukapheresis

In some embodiments, collection of mobilized mononuclear cell samples from healthy aged and young donors is performed at a qualified site. In some embodiments, participants are given an FDA approved, hematopoietic mobilizing agent on a daily basis at the currently recommended dosages (FIGS. 3A.2 and 4.2). In some embodiments, donors are given Filgrastim/Neupogen® (G-CSF) at 5-10 ug/kg by subcutaneous injection daily (e.g., for about 5 consecutive days). In some embodiments, G-CSF stimulates the bone marrow to produce a large number of hematopoietic and progenitor stem cells and mobilizes them into the peripheral blood stream. In some embodiments, CBCs to assess the response to the mobilizing agent are performed prior to mobilization and on the final day of mobilization prior to mononuclear cell (MNC) collection. In some embodiments, on a following day (e.g., the 6′ day), mobilized peripheral blood MNCs are collected by leukapheresis using a cell separator (FIG. 3A.3). In some embodiments, during leukapheresis, the collection of plasma and red blood cells is controlled to lower the collection of plasma and red blood cells relative to other blood factors. In some embodiments, leukapheresis is performed according to the manufacturer's instructions to process 18 L of blood at a flow rate of 50 to 100 mL per min. In some embodiments, mobilized MNC collections are performed for 4 to 6 hours for completion. In some embodiments, participants will generally have only one MNC collection performed immediately following mobilization. The product of 1 full MNC collection is referred to as a Leukopak. Fresh leukopaks should be processed within 24 hours of collection and should be stored at room temperature.

In some embodiments, the leukapheresis is performed using one or more of the following steps. In some embodiments, prior to collection, one or more pieces of the following information is gathered Documentation of the date of signed informed consent, venous assessment, CBC within 30 days of proposed collection date, Infectious Disease Markers testing statement (formal notification of known positive viral markers or known relevant communicable disease agents and diseases). In some embodiments, the mobilizing agent is administered according to each patient's doctor's instruction. In some embodiments, the entity that administers the mobilizing agent uses a predictive algorithm to calculate the optimum Total Blood Volume (TBV) required in order to meet the requested Mononuclear Cell (MNC) cell dose is used.

### Cell Processing—(To be Performed by GMP-Compliant Biobanking Facility)

Typical number of MNCs harvested from the leukapheresis procedure range from 25-50×10⁹ cells, with viability >95% and a collection volume of 300-400 mL (approximately 100×10⁶ cells/mL). In some embodiments, prior to cell processing, a sample of the Leukopak should be collected and cell number determined by counting with a hemocytometer. In some embodiments, further, cell viability should be determined using Turk's solution (as disclosed elsewhere herein). Additional evaluation of expression for the biomarkers CD45 and CD34 in the MNC collection can be made by flow cytometry to determine the percentage of leukocytes and hematopoietic stem/progenitor cells, respectively. In some embodiments, cells can be selected for those markers or others. In some embodiments, equal to or greater than about half of the plasma collected is removed from the resulting blood product. Typical number of CD34⁺ cells collected from mobilized leukapheresis range from 1-2×10⁷ cells per harvest dependent on the age of the donor, with young donors demonstrating greater yield. Cells are diluted in cryopreservation media at a 1:1 ratio to yield a final cell suspension of approximately 50×10⁶ cells/mL containing human serum albumin (HSA) and DMSO. In some embodiments, cells are then frozen (FIGS. 3A.4 and 4.3) using a programmable controlled rate freezer at a rate of −1° C./min to a temperature of −100° C. for transfer to liquid nitrogen storage.

### Transport

In some embodiments, as shown in FIGS. 3A.5 and 4.3-4.6, when mobilized peripheral blood (MPB) collections are scheduled, shipping logistics should be in place for proper sample handling and for an unbroken cold chain from collection to long-term storage. In some embodiments, a process to improve the viability of collected samples has been established. In some embodiments, blood sample shipments from the collection site to the cell processing site are performed using a validated cold storage cryoshippers (e.g., C₃™ Shipper, or cold shipper, etc.). In some embodiments, data loggers (e.g., SmartPak II™) are used to monitor the cell temperature over the course of transport.

### Example 2

Long Term Storage (Of Cells from Aged Donors)

The following describes the actions taken at FIG. 3A.5 (and FIG. 4.6) for long term storage of aged donor cells. In some embodiments, initial collections will include aged and young individuals for stem cell mobilization and leukapheresis (as described above). This section discloses procedures for use in the cryogenic processing of G-CSF-mobilized patient Leukopaks. In some embodiments, one or more of the following steps improve cell yield and viability, while allowing compatibility with regulatory guidelines and medical application. All procedures described in this section are for mobilized peripheral blood cells from aged donors (CRYO-MBR-A). One or more of the following steps may be omitted.

### Making Cryogenic Media for Total Nucleated Cells (TNCs)

In some embodiments, a cryogenic medium is prepared. In some embodiments, a cryogenic medium is prepared using HSA, DMSO, and normal saline.

Addition of Cryogenic Media to TNCs from Mobilized Peripheral Blood (MPBs)

In some embodiments, a centrifuge is equilibrated to 4° C. before processing the Leukopaks. In some embodiments, label cryogenic vials with Date, Patient ID, Vial Number, Patient Initials (patient descriptor, patient ID #, tube #, patient initials). For example: 12/05/18 PT-004-001-AC. In some embodiments, in ascending order, organize the cryogenic vial numbers into clean racks. Label cryogenic boxes with “rack number” location and “box number” (R# B#). In some embodiments, turn and leave on laminar flow hood, sterilize working surfaces with 70% ethanol and UV for a minimum of 10 minutes. In some embodiments, place labeled cryogenic boxes and labeled cryogenic vials under the laminar flow hood then turn on UV for a minimum of 20 minutes. In some embodiments, place the cryogenic vials on their designated boxes (e.g., 5 mL cryogenic vials should be placed into 5 mL cryogenic boxes, and 2 mL cryogenic vials should be placed into 2 mL cryogenic boxes). In some embodiments, place respective labeled cryogenic boxes (e.g., containing the respective labeled cryogenic vials) in the fridge (e.g., for 15 minutes or more). In some embodiments, ethanol spray and wipe down the chilled bead bucket and place under the laminar flow hood.

In some embodiments, the Leukopaks are removed from the shipping container and sprayed with ethanol thoroughly. In some embodiments, the Leukopaks are wiped down and placed under the sterile laminar flow hood. In some embodiments, with sterile scissors, cut the top portion of the Leukopaks and transfer a portion of the contents (e.g., equivalent to 1/10th of total Leukopak volume of MPBs each) into 10×50 mL sterile conical tubes. In some embodiments, spin down to pellet (e.g., at 300 g, at 4° C., for 10 minutes). In some embodiments, from each conical tube, remove 50% (approximately 20 mL per conical tube) of supernatant from each of the 10×50 mL conical tubes until left with only 50% of the initial total volume of supernatant and cell pellets. In some embodiments, with a 50 mL pipet and without disturbing the cell pellet, remove the remaining 20 mL supernatant and place it in a sterile 250 mL bottle. In some embodiments, loosen the pellets for all 10×50 mL conical tubes (e.g., with light tapping). In some embodiments, carefully resuspend pellets with 20 mL supernatant. In some embodiments, transfer the cell suspension into a single, sterile 500 mL bottle (total volume should be 200 mL). Keep the cell suspension chilled by placing the 500 mL bottle in the bucket with the cold beads.

In some embodiments, drop-wise add 200 mL of chilled cryogenic media into the cell suspension while gently shaking the bottle.

Aliquoting of Aged TNCs from Mobilized Peripheral Blood (MPBs) for Long Term Storage and Research and Development (R&D)

90% of the leukopak+cryogenic media, here in referred to as cryogenic suspension will be allocated for long term storage while 10% of this solution will be allocated for young donor evaluation.

90% of cryogenic suspension=360 mL; 72×5 mL vials

10% of cryogenic suspension=40 mL; 20×2 mL vials

In some embodiments, after gently mixing the cells with the cryogenic media, take a 1 mL aliquot and place at 4° C. for cell counting. In some embodiments, aliquot 90% of the cryogenic suspension into 5 mL aliquots within cryogenic vials. In some embodiments, place vials back into designated (5 mL cryogenic vials should be placed into 5 mL cryogenic boxes) boxes and place at 4° C. for 15 minutes. In some embodiments, aliquot 10% of the cryogenic suspension into 2 mL aliquots within cryogenic vials. In some embodiments, place vials back into designated boxes (2 mL cryogenic vials should be placed into 2 mL cryogenic boxes) and place at 4° C. for 15 minutes. Next, transfer vials from the previous steps, to the controlled rate freezer and the following programed protocol.

### Cryogenic Freezing of Cells

In some embodiments, set the Controlled-rate Freezing (CRF) program to an appropriate setting to achieve cryogenic freezing. In some embodiments, the temperature in the CRF chamber should be programmed to different temperature and cooling rates. In some embodiments, a controlled rate freezing program is utilized. In some embodiments, the program is custom and carried out with a controlled rate freezer to freeze the cells at an average rate of −1 degrees C. per minute from a starting temperature of 2-8 degrees C. to −100 degrees C. prior to deposition into a liquid nitrogen dewar.

In some embodiments, after the freezing, immediately transfer the boxes of cryogenic vials into the vapor phase of liquid nitrogen tank for long term cryopreservation. In some embodiments, perform cell count with Turk's solution and Trypan Blue exclusion. Record cell number, volume, concentration and viability. Between a minimum of 72 h and a maximum of 144 h, 1 vial of banked 5 mL cells should be thawed from donor sample to be tested.

Long Term Storage (Of Cells from Young Donors)

The following describes the actions taken at FIG. 3A.5 (and FIG. 4.6) for long term storage of young donor cells. In some embodiments, initial collections will include aged and young individuals for stem cell mobilization and leukapheresis (as described above). This section discloses procedures for use in the cryogenic processing of G-CSF-mobilized patient Leukopaks. In some embodiments, one or more of the following steps improve cell yield and viability, while allowing compatibility with regulatory guidelines and medical application. All procedures described in this section are for mobilized peripheral blood cells from young donors (CRYO-MBR-Y). One or more of the following steps may be omitted.

### Making Cryogenic Media for Total Nucleated Cells (TNCs)

To make about 250 mL of cryogenic media (MED-CRYO-100+), to a 500 mL sterile bottle add Normal Saline; HSA; and DMSO. Place solution at 4° C. until ready to be used.

Addition of Cryogenic Media to TNCs from Mobilized Peripheral Blood (MPBs)

In some embodiments, a centrifuge is equilibrated to 4° C. before processing the Leukopaks. In some embodiments, label cryogenic vials with Date, Donor ID, Vial Number, Patient Initials (patient descriptor, patient ID #, tube #, patient initials). For example: 12/16/18 DN-006-001-KG. In some embodiments, in ascending order, organize the cryogenic vial numbers into clean racks. Label cryogenic boxes with “rack number” location and “box number” (R# B#). In some embodiments, turn and leave on laminar flow hood, sterilize working surfaces with 70% ethanol and UV for a minimum of 10 minutes. In some embodiments, place labeled cryogenic boxes and labeled cryogenic vials under the laminar flow hood then turn on UV for a minimum of 20 minutes. In some embodiments, place the cryogenic vials on their designated boxes (e.g., 5 mL cryogenic vials should be placed into 5 mL cryogenic boxes, and 2 mL cryogenic vials should be placed into 2 mL cryogenic boxes). In some embodiments, place respective labeled cryogenic boxes (e.g., containing the respective labeled cryogenic vials) in the fridge (e.g., for 15 minutes or more). In some embodiments, ethanol spray and wipe down the chilled bead bucket and place under the laminar flow hood.

In some embodiments, the Leukopaks are removed from the shipping container and sprayed with ethanol thoroughly. In some embodiments, the Leukopaks are wiped down and placed under the sterile laminar flow hood. In some embodiments, with sterile scissors, cut the top portion of the Leukopaks and transfer a portion of the contents (e.g., equivalent to 1/10th of total Leukopak volume of MPBs each) into 10×50 mL sterile conical tubes. In some embodiments, spin down to pellet (e.g., at 300 g, at 4° C., for 10 minutes). In some embodiments, from each conical tube, remove 50% (approximately 20 mL per conical tube) of supernatant from each of the 10×50 mL conical tubes until left with only 50% of the initial total volume of supernatant and cell pellets. In some embodiments, with a 50 mL pipet and without disturbing the cell pellet, remove the remaining 20 mL supernatant and place it in a sterile 250 mL bottle. In some embodiments, loosen the pellets for all 10×50 mL conical tubes (e.g., with light tapping). In some embodiments, carefully resuspend pellets with 20 mL supernatant. In some embodiments, transfer the cell suspension into a single, sterile 500 mL bottle (total volume should be 200 mL). Keep the cell suspension chilled by placing the 500 mL bottle in the bucket with the cold beads.

In some embodiments, drop-wise add 200 mL of chilled cryogenic media into the cell suspension while gently shaking the bottle.

Some embodiments for specifications associated with Leukopaks include or exclude one or more of the following. I. Deliverables by StemExpress to Customer: 1. Fresh Mobilized Leukopak; Collected from donors sent to StemExpress by Customer; Dosing regimen of daily G-CSF (Neupogen) injections at a dose of 10 g/kg/day for 5 consecutive days, with leukapheresis collection on the 6^{th} day; Shipped in temperature-controlled packaging maintained between 2-8° C., and received by Customer within 24 hours of collection by FedEx First Overnight service or equivalent. 2. Certificate of Analysis a. To be sent electronically or via hard copy, and delivered prior to arrival of or with the Leukopak, respectively b. Should contain the following information i. Donor specification Age, Sex, Height, Weight, Ethnicity, Donor ID #. ii. Procedural specification: Needle IN time, Needle OUT time, Iii. Product specification: Total collection volume, Total nucleated cell count, Total nucleated cell viability, Percentage of CD45+ cells, Percentage of CD34+ cells, Low hematocrit, Low granulocytes. II. Acceptable Ranges for Product Specifications 1. Volume a. Minimum: 300 mL b. Maximum: 500 mL 2. Total Nucleated Cell Count a. Minimum: 20×10⁹ cells b. Maximum: None 3. Total Nucleated Cell Viability a. Minimum: 90% b. Maximum: 100% 4. Percentage of CD34+ cells a. Minimum: 1% b. Maximum: None.

Aliquoting of Aged TNCs from Mobilized Peripheral Blood (MPBs) for Long Term Storage and Research and Development (R&D)

90% of the leukopak+cryogenic media, here in referred to as cryogenic suspension will be allocated for long term storage while 10% of this solution will be allocated for young donor evaluation.

90% of cryogenic suspension=320 mL; 64×5 mL vials

10% of cryogenic suspension=80 mL; 40×2 mL vials

In some embodiments, after gently mixing the cells with the cryogenic media, take a 1 mL aliquot and place at 4° C. for cell counting. In some embodiments, aliquot 90% of the cryogenic suspension into 5 mL aliquots within cryogenic vials. In some embodiments, place vials back into designated (5 mL cryogenic vials should be placed into 5 mL cryogenic boxes) boxes and place at 4° C. for 15 minutes. In some embodiments, aliquot 10% of the cryogenic suspension into 2 mL aliquots within cryogenic vials. In some embodiments, place vials back into designated boxes (2 mL cryogenic vials should be placed into 2 mL cryogenic boxes) and place at 4° C. for 15 minutes. Next, transfer vials from the previous steps, to the controlled rate freezer and the following programed protocol.

### Cryogenic Freezing of Cells

In some embodiments, set the Controlled-rate Freezing (CRF) program to an appropriate setting to achieve cryogenic freezing. In some embodiments, the temperature in the CRF chamber should be programmed to different temperature and cooling rates. In some embodiments, a controlled rate freezing program is utilized. In some embodiments, the program is custom and carried out with a controlled rate freezer to freeze the cells at an average rate of −1 degrees C. per minute from a starting temperature of 2-8 degrees C. to −100 degrees C. prior to deposition into a liquid nitrogen dewar.

In some embodiments, after the freezing, immediately transfer the boxes of cryogenic vials into the vapor phase of liquid nitrogen tank for long term cryopreservation. In some embodiments, perform cell count with Turk's solution and Trypan Blue exclusion. Record cell number, volume, concentration and viability. Between a minimum of 72 h and a maximum of 144 h, 1 vial of banked 5 mL cells should be thawed from donor sample to be tested.

In some embodiments, long term storage (e.g., for donor, patient, or target cells) is performed at a temperature of equal to or less than about: −100° C., −150° C., −180° C., −190° C., −200° C., or ranges spanning and/or including the aforementioned values.

### Example 3

The following describes the treatment of aged cells with donor cells as shown in FIGS. 3A.6-3A.9 and 4.8. FIG. 5 provides an alternative depiction of the treatment steps, one or more of which may be omitted. For FIG. 5, a general schematic workflow illustrating the cell restoration process, including shipment, the entire process may take as little as 8 days. In some embodiments, the first 5 steps are at cell production facility, last 3 steps are at clinical facility. In some embodiments, the aged cells cultured with donor cells in the transwell are referred to as the composition AR-100.

### Cell Production, Processing and Clinical Infusion

This example describes embodiments for the production of therapeutic cells for infusion into a patient (e.g., PT-006), as shown in FIGS. 3A.6-3A.10 and 4.8-4.9. In some embodiments, the protocol utilizes cells from an aged donor (e.g., PT-006) and a young donor (e.g., Y03), where the aged donor is the patient and young donor is the donor. In some embodiments, cells produced are solely for infusion into patient PT-006. This batch represents the 1st batch produced for this patient and the patient's first infusion, herein referred to as PT-006.1.

### Equilibration Media

In some embodiments, 400 mL of equilibration media for young total nucleated cells is prepared. In some embodiments, supplemented Roswell Park Memorial Institute media (RPMI) is prepared. In some embodiments, to prepare Roswell Park Memorial Institute media (RPMI) supplemented with Penicillin Streptomycin & GlutaMAX-I, one or more of the following steps are used. In some embodiments, add of Penicillin Streptomycin and GlutaMAX-I to RPMI media bottle. In some embodiments, prepare DNase I and add. In some embodiments, place at −80° C. until ready to use then thaw overnight at 4° C. In some embodiments, place the 1 L bottle of media at 37° C. for 15 minutes.

In some embodiments, the equilibration medium is prepared using one or more of the following procedures. Section 1: Step 1: Supplementing RPMI with Pen Strep & Glutamine. Step 2: Dissolving DNAse in sterile water. Sterile filter using a 10 mL syringe and 0.2 um filter into a new sterile 15 mL conical tube. To each sterile 1 L disposable bottle add: H.S.A., RPMI (made in Step 1, section 1), DNAse I solution (made in Step 2, section 1) Step 4: Place the 1 L bottles of media at 37° C. for 15 minutes.

### Equilibration Media

In some embodiments, 650 mL of RPMI equilibration media for PT-006 cells is prepared. In some embodiments, the RPMI is supplemented with Penicillin Streptomycin & GlutaMAX-I. In some embodiments, DNase is dissolved in sterile water: In some embodiments, sterile filter (e.g., using a 10 mL syringe and 0.2 μm filter) into a new sterile 15 mL conical tube. In some embodiments, place at −20° C. until ready to use then thaw overnight at 4° C. In some embodiments, to one sterile 1 L disposable bottle add: H.S.A.; RPMI; DNase I solution. In some embodiments, place the 1 L bottles of media at 37° C. for 15 minutes.

In some embodiments, the equilibration medium is prepared using one or more of the following procedures. SECTION 2: 1000 mL of equilibration media for B0 TNCs Step 1: Supplementing RPMI with Pen Strep & Glutamine: Pen Strep and Glutamine to RPMI media bottle. Step 2: Dissolving DNAse in sterile water, Sterile filter using a 10 mL syringe and 0.2 um filter into a new sterile 15 mL conical tube. Step 3: To each sterile 1 L disposable bottle (2 total, approx. 500 mL/bottle) add: H.S.A. RPMI (made in Step 1, section 2) DNAse I solution (made in Step 2, section 2) Step 4: Place the 1 L bottles of media at 37° C. for 15 minutes.

### Restoration Media

In some embodiments, 1000 mL of cell restoration media is prepared (designated MED-CR-100). In some embodiments, to each Stem Span media bottle (2 total) add Penicillin Streptomycin and GlutaMAX-I. In some embodiments, place at 4° C. until ready to be used. In some embodiments, to make 1000 mL of cell restoration media, herein defined by the designation MED-CR-100+, to each MED-CR-100 bottle add MEM (minimum essential medium) Non-Essential Amino Acids Solution, Insulin-Transferrin-Selenium-Sodium Pyruvate, H.S.A. Place at 4° C. until ready to be used.

In some embodiments, the restoration medium is prepared using one or more of the following procedures. MED-CR-100: To make 1000 mL of cultivation media, to Stem Span media bottle add Pen Strep and Glutamine. Place at 4° C. until ready to be used. MED-CR-100+: To each MED-CR-100 bottle add: MEM Non-Essential Amino Acids Solution (100×) Insulin-Transferrin-Selenium-Sodium Pyruvate (ITS-A) (100×) H.S.A.

### Defrosting Cells and Equilibration

In some embodiments, the cells from long term storage are shipped to the cell restoration site in a frozen state (e.g., in liquid nitrogen, dry ice, etc.). As shown in FIG. 5, in some embodiments, the cells are defrosted (either cells from long term storage or newly prepared). In some embodiments, for defrosting and equilibrating the cells, one or more of the following steps can be used. In some embodiments, turn and leave on laminar flow hood, sterilize working surfaces with 70% ethanol and UV for a minimum of 10 minutes. In some embodiments, connect Aspirator system to vacuum source, label all 100 mm plates (30 plates for PT-006 cells, 20 plates for Y03 cells) then turn on UV for a minimum of 10 minutes. In some embodiments, take out equilibration media bottles from water bath and wipe down with 70% ethanol, then place under laminar flow hood.

Media distribution: In some embodiments, for the 30×100 mm plates allocated for PT-006 cells, add 19.5 mL of equilibration media to each plate, place on sterilized tray and place into incubator. In some embodiments, for the 20×100 mm plates allocated for Y03 cells, add 18 mL of equilibration media to each plate, place on sterilized tray and place into incubator.

### Equilibration of PT-006 Cells

The equilibration of the patient cells is shown in FIGS. 5, 6, and 7. In some embodiments, starting with PT-006 cells, take out 2 vials of cells from liquid nitrogen. In some embodiments, thaw 2 vials at a time by placing them into a water bath (previously set to 37° C.) for 5 minutes or until vials become visibly liquid. In some embodiments, wipe down the 2 vials with 70% ethanol before placing under laminar flow hood. In some embodiments, take out the 20×100 mm plates labeled with PT-006 (10 plates per vial of PT-006 cells) from incubator and place under the hood. In some embodiments, using a 5 mL pipette and without pipetting up and down, add 0.5 mL volume of cells drop wise onto each 100 mm dish. In some embodiments, gently place the 20×100 mm plates with cells in the incubator.

In some embodiments, take out the remaining 1 vial of PT-006 cells from liquid nitrogen. In some embodiments, thaw the 3rd vial by placing it into a water bath (previously set to 37° C.) for 5 minutes or until vial become visibly liquid. In some embodiments, wipe down the vials with 70% ethanol before placing under laminar flow hood. In some embodiments, take out the 10×100 mm plates labeled with PT-006 (10 plates per vial of PT-006 cells) from incubator and place under the hood. In some embodiments, using a 5 mL pipette and without pipetting up and down, add 0.5 mL volume of cells drop wise onto each 100 mm dish. In some embodiments, gently place the 10×100 mm plates with cells in the incubator. In some embodiments, a timer is set for about: 1 hour, 2 hours, 3 hours, 4 hours, 5 hours, or ranges spanning and/or including the aforementioned values.

### Equilibration of Y03 Cells

In some embodiments, 20 vials of Y03 cells are thawed. In some embodiments, take 10 vials (up to 15 vials maximum) at a time out of the liquid nitrogen container and to place them at −80° C. (first set) 15±3 minutes. In some embodiments, thaw 5 vials at a time by placing them into a water bath (previously set to 37° C.) for 5 minutes or until vial contents become visibly liquid. In some embodiments, wipe down the 5 vials with 70% ethanol before placing them under the laminar flow hood. In some embodiments, take out 5×100 mm plates labeled with Y03 cells (1 plate per vial of Y03 cells) from incubator and place under the laminar flow hood. In some embodiments, using a 5 mL pipette and without pipetting up and down, add 2 mL volume of cells drop wise onto each 100 mm dish. In some embodiments, gently place the 5×100 mm plates with cells in the incubator. In some embodiments, repeat the above steps 17-20 for the remaining 5 vials to prepare the first set.

In some embodiments, take 10 more vials of Y03 cells (second set) out of the liquid nitrogen container and place them in the −80° C. freezer for 15±3 minutes. In some embodiments, thaw 5 vials at a time by placing them into a water bath (previously set to 37° C.) for 5 minutes or until vial contents become visibly liquid. In some embodiments, wipe down the 5 vials with 70% ethanol before placing them under the laminar flow hood. In some embodiments, take out 5×100 mm plates labeled with Y03 cells (1 plate per vial of Y03 cells) from incubator and place under the laminar flow hood. In some embodiments, using a 5 mL pipette and without pipetting up and down, add 2 mL volume of cells drop wise onto each 100 mm dish. In some embodiments, gently place the 5×100 mm plates with cells in the incubator. In some embodiments, repeat on the remaining 5 vials for the second set. In some embodiments, a timer is set for about: 1 hour, 2 hours, 3 hours, 4 hours, 5 hours, or ranges spanning and/or including the aforementioned values. FIGS. 6 and 7 show two methods for equilibrating stored cells with different amounts of cells per storage vessel. As shown in FIG. 6, where cells are more concentrated (e.g., equal to or greater than about 1×10⁹ cells are present per vial), a smaller number of vials is used per clinical treatment of aged cells (e.g., about 4 vials of Y03 cells are thawed to provide a clinical treatment). As shown in FIG. 7, where cells are more concentrated (e.g., equal to or less than about 1×10⁹ cells are present per vial—in this case 2.8×10⁹ cells per vial), a larger number of vials is used per clinical treatment of aged cells (e.g., about 40 vials of Y03 cells are thawed to provide a clinical treatment).

Preparation during the Equilibration hold: In some embodiments, use 70% Ethanol to wipe the laminar flow hood and U.V. for a minimum of 10 minutes. In some embodiments, spray down 6 well plates, transwell inserts, and 50 mL conical tubes then place all under the laminar flow hood. U.V. for a minimum of 10 minutes. In some embodiments, label all plates, and conical tubes. In some embodiments, take out the cell culture media and place it at 37° C. for about 30±2 minutes.

After the Equilibration: In some embodiments, take out plates for PT-006 cells from the incubator, place on sterilized tray, and place under the laminar flow hood. In some embodiments, pipette up and down (cells+media from respective 100 mm plates) and place into respective sterile labeled 50 mL conical tubes (Note: In some embodiments, the volume of 2 plates can be placed into 1 conical tube. Thus, will have 15×50 mL conical tubes of PT-006 cells). In some embodiments, spin down (at 300 g, room temperature, and for 10 minutes). In some embodiments, remove supernatant from all 50 mL conical tubes until left with only cell pellets. In some embodiments, lightly tap and loosen pellets for all 15×50 mL conical tubes. In some embodiments, add Stem Span culture media and place back in incubator.

In some embodiments, take out Y03 plates from the incubator, place on sterilized tray, and place under the laminar flow hood. In some embodiments, pipette up and down (cells+media from respective 100 mm plates) and place into respective labeled 50 mL tubes (Note: In some embodiments, volume of 2 plates can be placed into 1 conical tube. Thus 10×50 mL conical tubes of Y03 cells will be generated). In some embodiments, spin down (at 300 g, room temperature, and for 10 minutes). In some embodiments, remove supernatant from all 50 mL conical tubes until left with only cell pellets. In some embodiments, lightly tap and loosen pellets for all 10×50 mL conical tubes, and then add MED-CR-100+(SS+) culture media and place back in incubator.

In some embodiments, both PT-006 and Y03 cells are ready to be counted (e.g., with Turk's and Trypan blue). In some embodiments, label disposable culture tubes with respective sample name. In some embodiments, add Turk's/Trypan on each tube. In some embodiments, add cells and mix well. In some embodiments, place the remainder of the cells back into incubator. In some embodiments, proceed to counting. In some embodiments, after counting the cells, then proceed to seeding cells onto 6 well plates and transwell inserts based on desired seeding density (20-30×10⁶ cells per well).

In some embodiments, the cell counting is performed as follows: Prepare an appropriate dilution of cells in Turk's solution. Dilution factor should be empirically determine based on packed cell pellet volume to yield a reasonable number of cells per field of vision during step 4. Prepare a hemocytometer by first cleaning the chamber surface with 70% alcohol. Wipe dry. Position the coverslip over the chambers. Carefully transfer 10 uL volume of the Turk's/cell solution to each chamber using a pipette. Do not over- or underfill. Begin by counting the cells in one chamber. Count all cells in each 1 mm square of each chamber. If cells are on the border outlining each square, count only the cells on the top and left border of the square. NOTE: Each square has a total volume of 0.1 mm³ (or 10\hat{ }−4 cm³, which is approximately equivalent to 10\hat{ }−4 mL). Determine the cell count (cells per mL) as follows: Average cell count per square×dilution factor×10 4=cell count per mL

### Transwell Culture

As shown in FIGS. 3A.6, 5, 8, and 9, in some embodiments, a transwell culture is performed. *Note 1: In some embodiments, total volume of cell suspension and culture media=3.5 mL for each inner well and Transwell insert. In some embodiments, place required volume of PT-006 cells to create a cell suspension equivalent to 20-30×10⁶ cells per inner well. In some embodiments, supplement this cell suspension volume with MED-CR-100+ to a final volume of 3.5 mL per inner well. In some embodiments, with sterile tweezers place transwell inserts into inner wells. In some embodiments, place volume of Y03 cells cell suspension equivalent to same number of cells used above in the inner well per transwell insert. In some embodiments, supplement this cell suspension volume with restoration media to a final volume of 3.5 mL per transwell insert. In some embodiments, close all plate lids and place into incubator for culture.

### Day 3 Mid-Cultivation Media Supplementation

In some embodiments, after three days, place MED-CR-100+ bottle into water bath set to 37° C. for 30±2 minutes. Ethanol wipe and UV laminar flow hood for a minimum of 10 minutes and maximum of 12 minutes. After media is warmed to 37±1C, take out plates from incubator and place into laminar flow hood. Take 200 μL of cell suspension from an arbitrary well and place in sterile 2 mL microcentrifuge tube and place aside. Use this aliquot for in-process testing (SOP-TEST-001 (InvivoGen PlasmoTestMycoplasma Detection Kit), -002 (cell viability Trypan Blue test); Form-001, -002).

In some embodiments, without removing the transwell inserts from the plates, supplement each well and transwell insert with 1 mL of 37±1° C. MED-CR-100+. Visually check for any color change in the media, or any presence of contamination. If no contamination detected, place all plates back into incubator for the remainder of the culture.

### Day 6 of Culture

In some embodiments, place MED-CR-100+ bottle into water bath set to 37° C. for a minimum of 30±2 minutes. In some embodiments, use 70% Ethanol to wipe the laminar flow hood and U.V. for a minimum of 10 minutes. In some embodiments, take out MED-CR-100+ bottle from water bath and wipe down with 70% ethanol, then place under the laminar flow hood. In some embodiments, take out plates from incubator and place under the laminar flow hood. In some embodiments, take 200 μL of cell suspension from an arbitrary well and place in sterile 2 mL microcentrifuge tube and place aside. In some embodiments, use this aliquot for in-process testing (SOP-TEST-001, -002; Form-001, -002).

In some embodiments, check for any color change in the media, or any presence of contamination. If no contamination detected, place all plates back into incubator for the remainder of the culture. After a minimum of 16 h and a maximum of 24 h, the results of the sterility test will be available. If results of sterility test are negative for contaminants, proceed to the next steps of the protocol.

### Preparing Wash/Infusion Buffer for Final Formulation

In some embodiments, prepare 1000 mL of wash/infusion buffer, herein referred to as MED-WI-100. In some embodiments, to make 1000 mL of wash/infusion buffer, herein referred to as MED-WI-100, combine normal saline and human serum albumin into each of 2×1 L sterile disposable bottle.

### Processing, Washing and Final Formulation of the Cell Product

The following describes procedures performed as shown in FIGS. 3A.7, 4.8, 5, and 8. In some embodiments, place MED-WI-100 bottle into water bath set to 37° C. for 30±2 minutes. In some embodiments, place a 500 mL of normal saline, 0.9% sodium chloride injection bag to pre-warm on an injection bag. Ethanol wipe and UV laminar flow hood for a minimum of 10 minutes and maximum of 12 minutes. In some embodiments, after buffer is warmed to 37±1° C., take out bottle from water bath, wipe down with 70% ethanol, then place under the laminar flow hood. In some embodiments, collect all inner well media/cells and transfer 40 mL of media/cells into a plurality of 50 mL conical tubes noting the actual number. There will be ˜200-600 mL total volume of media/cells. Spin down at 300 g, room temperature, and for 10 minutes. Remove supernatant from all 50 mL conical tubes until left with only cell pellets.

In some embodiments, lightly tap and loosen pellets for all 50 mL conical tubes. In some embodiments, add 4 mL of wash buffer to each loose pellet. In some embodiments, with a 5 mL pipette, gently pipette up and down to loosen pellet and then combine volume of all the tubes into 4×50 mL conical tubes (11 mL of wash buffer/cells). In some embodiments, to each conical tube add 39 mL of MED-WI-100 (wash buffer). In some embodiments, spin down (at 300 g, room temperature, and for 10 minutes). In some embodiments, remove supernatant from all 4×50 mL conical tubes until left with only cell pellets. In some embodiments, lightly tap and loosen pellets for all 4×50 mL conical tubes, and then add 5 mL of MED-WI-100 (wash buffer) to each loose pellet. Combine volume of each conical tube into 1×50 mL conical tube [20 mL total volume of cells/MED-WI-100 (Wash buffer)].

In some embodiments, at this point, cells are ready to be counted with Turk's and Trypan blue solutions. In some embodiments, counting steps include one or more of: Take an aliquot of cells/infusion buffer and place into 2 disposable; To tube 1 add Turk's solution and to tube 2 add Trypan blue solution and mix well; Proceed to counting. In some embodiments, keep the remainder of the cells under the laminar flow hood. In some embodiments, after determining the final count, remove 25-50×10⁶ cells and set aside for cryopreservation for future experiments and testing. In some embodiments, adjust cell/infusion concentration to 10×10⁶ cells/mL (expected cell yield is 5-10×10⁸ Cells in 20-200 ml of infusion buffer, respectively) with MED-WI-100 to prepare the Final Volume for Infusion.

In some embodiments, as shown in FIG. 9, final in-process testing is performed (SOP-TEST-002, Form-002, -200). If the minimum viability of 65% from SOP-TEST-002 is obtained, proceed to next step. If this minimum value is not obtained, refer to SOP-LAB-003 (SOP test for cell identity in mobilized peripheral blood through cell surface antibody staining and flow cytometry). Cell quality can be tested using a cell vitality assay (e.g., a two color fluorescence assay that distinguishes metabolically active cells from injured cells and dead cells). Cell potency can be tested using a clonogenic assay, which utilizes specialized growth media to test the number of colony-forming units (CFUs) within a culture containing hematopoietic stem and progenitor cells. A mobilized blood culture yielding large numbers of CFUs would be considered highly potent, while one yielding few or no CFUs would be considered minimally potent. The cell identity, quality, viability, vitality testing and other “SOP” testing can be performed at any time, including after harvesting of cell, after cryogenic treatment, after exposure to other cells using a transwell plate, prior to infusion, etc. FIGS. 9B-9D provide alternative embodiments for logistics in the clinical processing of cells.

Examine final formulated product for any evidence of cell aggregation or clumping. *Note: Evidence of aggregation or clumping during final filling may be grounds to discontinue the protocol if no resolution can be implemented. In some embodiments, this is important during the filling of the final cell suspension into syringes and infusion bag. In some embodiments, if cell aggregation or clumping is observed, cell suspensions can be passed through a sterile cell strainer (100 μm—Fisher 22363549) to clarify the product. In some embodiments, if aggregates remain, discuss with clinical staff the risk to patient or no further action. In some embodiments, if the clinical risk is low, proceed. At this point, cells are ready to be infused. Proceed to transferring final volume into infusion bags for patient infusion.

### Preparation of the Infusion Bag

In some embodiments, at this time, note the total volume of cell product above and the Final Volume for Infusion and subtract to determine Volume of Normal Saline, 0.9% sodium chloride injection that should be added. In some embodiments, remove the pre-warmed normal saline, 0.9% sodium chloride, 500 mL injection bag. In some embodiments, clean with ethanol and place into hood and thoroughly clean the self-healing port with a 70% isopropanol cleaning wipe and allow 10 seconds to dry. In some embodiments, clean the self-healing port of an empty 250 mL Infusion bag with a 70% isopropanol cleaning wipe and allow 10 seconds to dry. In some embodiments, using a sterile 60 mL syringe and 18 G needle, withdraw a volume of saline (the Volume of Normal Saline above) from the pre-warmed 500 mL saline injection bag and load into the sterilized empty 250 mL bag. In some embodiments, invert empty 250 mL infusion bag and load pre-warmed saline through the self-healing port. In some embodiments, repeat with same syringe and needle until the desired volume has been achieved.

In some embodiments, prepare a sterile 60 mL syringe with a sterile 18G needle and insert into the same self-healing port of the 250 mL infusion bag. In some embodiments, using a 5 or 10 mL pipette, back load the cell product (resuspended in MED-WI-100) into a sterile, un-plunged 60 mL syringe and allow the cell product to gravity flow into the 250 mL infusion bag. In some embodiments, repeat these steps until all of the cell product is injected. Note the Final concentration of HSA in saline bag after filling.

In some embodiments, remove the syringe and slowly rotate the 250 mL injection bag to mix the cell product with the saline. In some embodiments, observe if any clumping or aggregation occurs. If no clumping or aggregation is detected, the cell product is now ready to be transferred to the clinician.

### Patient Infusion

As shown in FIG. 3A.9, the cells can then be infused with AR-100. In some embodiments, after mixing the content of the 250 mL saline bag, the bag is spiked on one spike of the blood tubing and the 500 mL bag of saline is spiked on the other. In some embodiments, the blood tubing is primed with the 500 mL bag of saline. In some embodiments, the Y tubing to the 500 mL saline bag is clamped once the tubing is primed. In some embodiments, the blood tubing is then connected to the lowest Y tubing site of the patient's IV tubing and the 250 mL bag of saline+cell product is infused over 60 to 90±2 minutes.

In some embodiments, once the 250 mL bag is empty, it is gravity fed by the 500 mL bag to clear the bag and tubing (flushing all cells out of the 250 mL bag and Y tubing); this is allowed to infuse into the patient. In some embodiments, once flushed and emptied the Y tubing to the 250 mL bag is clamped. In some embodiments, the Y tubing to the 500 mL bag is then opened and approximately 150 to 200 mL of pure saline is flushed to clear lines and make sure all cells are infused.

### Make 10 mL of Cryogenic Media for PT-006 Cells

In some embodiments, cryogenic media is prepared. In some embodiments, to make 10 mL of cryogenic media, herein defined by the designation MED-CRYO-100, add Dimethyl Sulfoxide (DMSO, Fisher Scientific, catalog #BP231-100) and HSA into a 50 mL conical tube. Place solution at 4° C. until ready to be used.

### Freezing of PT-006 Cells

In some embodiments, label cryogenic vials with cell type, patient number, number of cells, date and operator's initials. In some embodiments, place cryogenic freezing container in the fridge for 15 minutes. In some embodiments, spin down the 25-50×10⁶ PT-006 cells set aside above (e.g., at 300 g, room temperature, and for 10 minutes). In some embodiments, take out cryogenic freezing container and MED-CRYO-100 media from the fridge, spray with 70% ethanol and place under the sterile hood. In some embodiments, remove supernatant from conical tube until left with only cell pellet. In some embodiments, lightly tap conical tube to loosen pellet, and then pipette MED-CRYO-100 media to resuspend cells; and immediately pipette into labeled cryogenic vial, for a final concentration of approximately 25-50×10⁶ cells/mL. In some embodiments, place the cryogenic vial in the cryogenic freezing container and place immediately at −80° C.

### Example 4

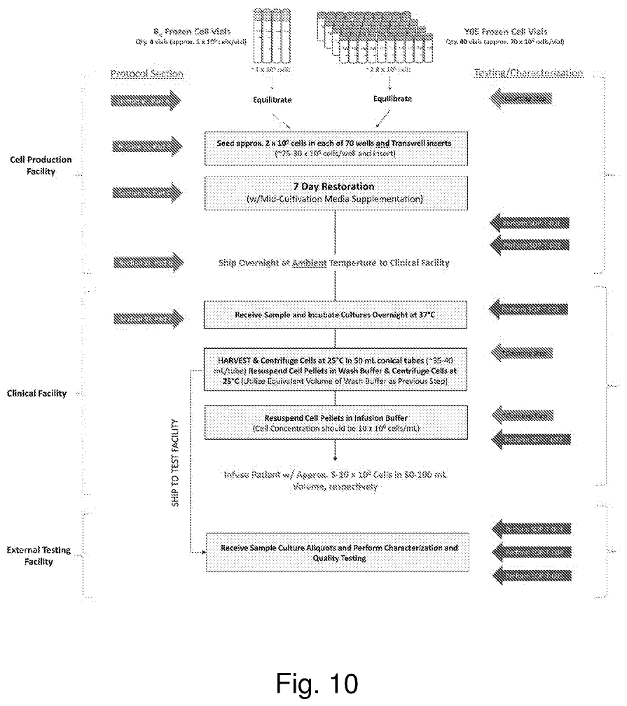

The following is an additional embodiment of a method of clinical cell production. In some embodiments, a purpose of the following was to clinically translate existing research-grade transwell restoration methods by incorporating protocol modifications that will enable compatibility with regulatory guidelines and medical application. FIG. 10 provides an additional embodiment of the cell restoration and/or treatment process.

In some embodiments, the restoration and/or treatment process may make use of one or more of the following pieces of equipment: Bench Top centrifuge for 15 mL and 50 mL conical tubes (need 4 buckets to hold 50 mL conical tubes); −200 C freezer; Light microscope; Vortex; 4° C. refrigerator; CO2 incubator (5%); Laminar flow biosafety cabinet with UV; Liquid Nitrogen Storage; Water Bath; Vacuum source; Analytical Scales; Hemocytometer; Aspirator system; Autoclave if available; Reverse Osmosis H₂O purification system if available; Scientific Calculator; Sterile Trays; Kim Wipes (Kimberly Clark; 06-666-A), Pipette Tips 1000 uL (Rainin Instrument, LLC; SR-L1000F), Pipette Tips 200 uL (Rainin Instrument, LLC; SR-L200F), Pipette Tips 20 uL (Rainin Instrument, LLC; SR-L10F), Serological Pipettes 5 mL (Fisher Scientific; 13-678-12D), Serological Pipettes 10 mL (Fisher Scientific; 13-678-11E), Serological Pipettes 25 mL (Fisher Scientific; 13-678-14B), Serological Pipettes 50 mL (Fisher Scientific; 13-678-14C), Sterile syringe filters 0.2 um (Thermo Scientific; 09-740-113), Syringes 10 mL (Becton Dickinson; 14-823-16E), 15 mL Sterile Conical tubes (Corning; 430791), 50 mL sterile Conical tubes (Corning; 430828) Qty. 3 cases, Tissue Culture Plate—6 well (Corning; 353502) Qty. 15, Transwell inserts 0.4 um (Corning; 353090) Qty. 80, Medium Gloves (Fisher Scientific; 19-050-550B), Ethanol Solution 70%, Molecular Biology Grade 4 L (Fisher Scientific; BP82014), Tube Rack 15 mL conical tube (Fisher Scientific; 14-791-6D), Tube Racks 50 mL conical tube (Fisher Scientific; 14-791-6B), Disposable Sterile 1 L bottles (Fisher Scientific; 09-761-11) Qty. 10, Fisherbrand Plastic Petri Dishes (Fisher Scientific; S33580A), Disposable culture tubes 12×75 mm (VWR; 10029-154), Disposable Pasteur pipet 9″ (VWR; 14672-380), Parchment paper 6×6 (Fisher Scientific; 09-898-12C), Marking Pens (Fisherbrand; 13-379-4), Spray Bottles for Ethanol, T25 tissue culture flasks (Corning; 10-126-10-24EA) Qty. 10, Pipette fillers, Sterile Tweezers, Biohazard bags, Cell Counter, Timers, Polar Tech 266C Thermo Chill Insulated Carton with Foam Shipper, Sterile cell strainer 100 μm (Fisher 22363549),

In some embodiments, reagents include: Pen/Strep (Sigma; P0781) (100×) Qty. 2, 0.4% Trypan Blue (Sigma; T8154) Qty. 1, Dnase I (Worthington; LS006362) Qty. 9; 7.5 mg/ampule), 70% ethanol (Fisher Scientific; BP82014), Turk's Solution (Millipore)) Qty. 1, Glutamine (Sigma) (100×) Qty. 2, StemSpan (Stem Cell Technologies) Qty. 3 bottles, RPMI-1640 (Gibco; 21870-0786) Qty. 3 bottles, Sterile deionized water, Sterile normal saline, HSA (Irvine Scientific, Cat#9988) Qty. 13 bottles, Sterile deionized water, Sterile normal saline, HSA (Irvine Scientific, Cat#9988) Qty. 2 bottles, 70% ethanol (Fisher Scientific; BP82014), 0.4% Trypan Blue (Sigma; T8154) Qty. 1, Turk's Solution (Millipore) Qty. 1,

### Defrosting Cells and Equilibration

In some embodiments, turn on laminar flow hood sterilize with 70% ethanol and UV for 10 minutes. In some embodiments, connect Aspirator system to vacuum source, label all 100 mm plates (40 plates for B_{O} cells (patient cells), 45 plates for Y05 cells) then turn on UV for 10 minutes. Take out equilibration media bottles from water bath and wipe down with 70% ethanol, then place under laminar flow hood. Media distribution: For the 40×100 mm plates allocated for B_{O} cells, add 19.5 mL of equilibration media to each plate, place on sterilized tray and place into incubator. For the 45×100 mm plates allocated for Y05 cells, add 18 mL of equilibration media to each plate, place on sterilized tray and place into incubator.

### B_{O} Cells

In some embodiments, starting with B_{O} cells, take out 4 vials of cells (or an appropriate amount of cells) from liquid nitrogen and place them at −20° C. While specific numbers of vials etc. are used herein, other values are envisioned dependent on the number of cells desired. Note from this point on, need to work fast since these cells are time and temperature sensitive. In some embodiments, thaw 2 vials (or an appropriate amount of cells) at a time by placing them into a water bath (previously set to 37° C.) for 5 minutes or until vials become visibly liquid). In some embodiments, wipe down the 2 vials with 70% ethanol before placing under laminar flow hood. In some embodiments, take out the 20×100 mm plates labeled with B_{O} (10 plates per vial of B_{O} cells) from incubator and place under the hood. In some embodiments, using a 5 mL pipette and without pipetting up and down, add 0.5 mL volume of cells drop wise onto each 100 mm dish. In some embodiments, gently place the 20×100 mm plates with cells in the incubator. In some embodiments, from the −20° C. freezer take out the remaining 2 vials of B_{O} cells and place them into a water bath (previously set to 37° C.) for 5 minutes or until vials become visibly liquid). In some embodiments, wipe down the 2 vials with 70% ethanol before placing under laminar flow hood. In some embodiments, take out the remaining 20×100 mm plates labeled with B_{O} (10 plates per vial of B_{O} cells) from incubator and place under the laminar flow hood. In some embodiments, using a 5 mL pipette and without pipetting up and down, add 0.5 mL volume of cells drop wise onto each 100 mm dish. Gently place the 20×100 mm plates with cells in the incubator.

### Y05 Cells

In total, an appropriate number of Y05 cells should be thawed (e.g., 45 vials). While specific numbers of vials etc. are used herein, other values are envisioned dependent on the number of cells desired. In some embodiments, it is recommended to take 15 vials at a time out of the liquid nitrogen container and to place them at −20° C. (First set). Note from this point on, need to work fast since these cells are time and temperature sensitive. In some embodiments, thaw 5 vials at a time by placing them into a water bath (previously set to 37° C.) for 5 minutes or until vials become visibly liquid. In some embodiments, wipe down the 5 vials with 70% ethanol before placing them under the laminar flow hood. Take out 5×100 mm plates labeled with Y05 (1 plate per vial of Y05 cells) from incubator and place under the laminar flow hood. In some embodiments, using a 5 mL pipette and without pipetting up and down, add 2 mL volume of cells drop wise onto each 100 mm dish. In some embodiments, gently place the 5×100 mm plates with cells in the incubator. Repeat steps for the remaining 10 vials for the first set. In some embodiments, take 15 more vials of Y05 cells (Second set) out of the liquid nitrogen container and place them at −20° C. freezer. In some embodiments, from the −20° C. freezer, thaw 5 vials at a time by placing them into a water bath (previously set to 37° C.) for 5 minutes or until vials become visibly liquid. In some embodiments, wipe down the 5 vials with 70% ethanol before placing them under the laminar flow hood. In some embodiments, take out 5×100 mm plates labeled with Y05 (1 plate per vial of Y05 cells) from incubator and place under the laminar flow hood. In some embodiments, using a 5 mL pipette and without pipetting up and down, add 2 mL volume of cells drop wise onto each 100 mm dish. In some embodiments, gently place the 5×100 mm plates with cells in the incubator. Repeat steps for the remaining 10 vials for the first set.

In some embodiments, take the final 15 vials of Y05 cells (Third set) out of the liquid nitrogen container and place them at −20° C. freezer. In some embodiments, from the −20° C. freezer, thaw 5 vials at a time by placing them into a water bath (previously set to 37° C.) for 5 minutes or until vials become visibly liquid. In some embodiments, wipe down the 5 vials with 70% ethanol before placing them under the laminar flow hood. In some embodiments, take out 5×100 mm plates labeled with Y05 (1 plate per vial of Y05 cells) from incubator and place under the laminar flow hood. In some embodiments, using a 5 mL pipette and without pipetting up and down, add 2 mL volume of cells drop wise onto each 100 mm dish. Gently place the 5×100 mm plates with cells in the incubator. In some embodiments, repeat steps for the remaining 10 vials for the first set. In some embodiments, set a timer for 1, 2, 3, 4, 5, or more hours.

### Preparation During the Equilibration

In some embodiments, equilibration is performed for a period of about: 1 hour, 2 hours, 3 hours, 4 hours, 5 hours, or ranges spanning and/or including the aforementioned values. In some embodiments, use 70% Ethanol to wipe the laminar flow hood and U.V. for 10 minutes. In some embodiments, spray down 6 well plates, transwell inserts, and 50 mL conical tubes then place all under the laminar flow hood. U.V. for 10 minutes. In some embodiments, label all plates, and conical tubes. Take out the cell culture media and place it at 37° C. for 30 minutes.

### After the Equilibration

In some embodiments, take out plates for B_{O} cells from the incubator, place on sterilized tray, and place under the laminar flow hood. In some embodiments, pipette up and down (cells+media from respective 100 mm plates) and place into respective sterile labeled 50 mL conical tubes (note: Volume of 2 plates can be placed into 1 conical tube. In some embodiments, thus will have 20×50 mL conical tubes of B_{O} cells.) Spin down at 300 g, room temperature, and for 10 minutes. In some embodiments, remove supernatant from all 50 mL conical tubes until left with only cell pellets. In some embodiments, lightly tap and loosen pellets for all 20×50 mL conical tubes, and then add complete culture media and place back in incubator. In some embodiments, take out Y05 plates from the incubator, place on sterilized tray, and place under the laminar flow hood. In some embodiments, pipette up and down (cells+media from respective 100 mm plates) and place into respective labeled 50 mL tubes (note: Volume of 2 plates can be placed into 1 conical tube. In some embodiments, thus will have 18×50 mL conical tubes of Y05 cells.) Spin down at 300 g, room temperature, and for 10 minutes. In some embodiments, remove supernatant from all 50 mL conical tubes until left with only cell pellets. In some embodiments, lightly tap and loosen pellets for all 18×50 mL conical tubes, and then add complete culture media and place back in incubator. In some embodiments, at this point, both B₀ and YO5 cells are ready to be counted with Turk's and Trypan blue. In some embodiments, label disposable culture tubes with respective sample name. In some embodiments, add Turks/Trypan on each tube. In some embodiments, add cells and mix well. Place the remainder of the cells back into incubator. In some embodiments, proceed to counting. In some embodiments, after counting the cells, then proceed to seeding cells onto 6 well plates and transwell inserts based on desired seeding density (20-30M cells per well).

### Transwell Culture

In some embodiments, *Note 1: Total volume of cell suspension and culture media=3.5 mL for each inner well and Transwell insert. *Note 2: Have ˜70 wells and 70 transwell inserts, for 12×6-well plates Label all plates (1-12); Place required volume of B_{O} cells cell suspension equivalent to 20M-30M cells per inner well; and supplement this cell suspension volume with restoration media to a final volume of 3.5 mL per inner well. With sterile tweezers place transwell inserts into inner wells (70 total). Place required volume of Y05 cells cell suspension equivalent to same number of cells used in step 2 per transwell insert; and supplement this cell suspension volume with restoration media to a final volume of 3.5 mL per transwell insert. Close all plate lids and place into incubator for culture.

### Day 4 Mid-Cultivation Media Supplementation

In some embodiments, place restoration media bottle into water bath set to 37° C. for 30 minutes. Ethanol wipe and UV laminar flow hood for 10 minutes. After media is warm, take out plates from incubator and place into laminar flow hood. Without removing the transwell inserts from the plates, supplement each well and transwell inserts with 1 mL of warm restoration media. Place all plates back into incubator for the remainder of the culture. *Note: This is also one of the times one should visually check for any color change in the media, or any presence of contamination.

### Day 7 of Culture

One or more of the following steps can be performed. Preparations before taking restored cells out of the incubator: Make sure laminar flow hood has been sterilized with ethanol and sterilized by UV. Make sure the aspirator system is connected along with the Pasteur pipet and ready to go. Make sure sterile 50 mL conical tubes are already placed under the laminar flow hood. Make sure tweezers have been sterilized and placed under the laminar flow hood. Make sure the centrifuge is turned on and ready to go. Make sure the pipette filler has been charged and ready to operate. Place MED-CR-100+ bottle into water bath set to 37° C. for 30 minutes.

Procedure: One or more of the following steps can be performed. Take out MED-CR-100+ media bottle from water bath and wipe down with 70% ethanol, then place under the laminar flow hood. Take out plates from incubator and place under the laminar flow hood. Take 1 mL of cell suspension from an arbitrary well and place in sterile 2 mL microcentrifuge tube and place aside. Use this aliquot for in-process testing (see FIG. 10: ARMT-SOP-T-001 and -002). If needed, adjust volumes with Stem Spam culture media to completely fill transwells (avoid air bubbles). Seal transwells with sterile parafilm (see Appendix illustration), place lid back onto plate and then seal entire plate securely in sterile parafilm for transport. Package into Polar Tech 266C Thermo Chill Insulated Carton with Foam Shipper and seal properly. Cells are ready to be shipped. Transportation of cells should occur by private courier so that they are in-transit for less than 12 hour. Shipment from cell production facility and receipt at clinical facility should occur on the same day. Take the 1 mL of cell suspension and spin down at 300 g, for 10 minutes at room temperature. Remove supernatant from 2 mL microcentrifuge tube until left with only cell pellets. Lightly tap and loosen pellets and then add complete culture media. At this point, proceed to counting with Turk's and Trypan blue. Label disposable culture tubes with respective sample name. Add Turks/Trypan on each tube. Add cells and mix well. Proceed to counting.

### Receipt of Cells, Sample Processing, Washing, Final Cell Formulation & Patient Infusion

Preparations for removing cells from packaging before infusion: Make sure laminar flow hood has been sterilized with ethanol and sterilized by UV. Make sure the aspirator system is connected along with the Pasteur pipet and ready to go. Make sure sterile 50 mL conical tubes are already placed under the laminar flow hood. Make sure the centrifuge is turned on and ready to go. Make sure the pipette filler has been charged and ready to operate.

Place Wash buffer bottle into water bath set to 37° C. for 30 minutes; then bring to room temperature. Place Infusion buffer bottle into water bath set to 37° C. for 30 minutes; then bring to room temperature. Place Stem Span media bottle into water bath set to 37° C. for 30 minutes.

### Procedure:

Take out Stem Span media bottle from water bath and wipe down with 70% ethanol, then place under the laminar flow hood. Remove plates from packaging, wipe with 70% ethanol and place under the laminar flow hood. With tweezers gently take out transwell inserts and throw away in biohazard bins. Remove 1.0 mL of cell suspension from 5 random wells and perform sterility testing. Place cultures in 37° C. cell culture incubator overnight, for harvesting the next day. Collect all inner well media/cells and transfer 40 mL of media/cells into 10 or 11×50 mL conical tubes. *Note: Will have ˜400-450 mL total volume of media/cells.

Ship 5 mL of cell culture suspension to external testing facility for identity, quality and potency testing. If results of sterility test are negative for contaminants, proceed.

Spin down at 300 g, room temperature, and for 10 minutes. Remove supernatant from all 50 mL conical tubes until left with only cell pellets. Lightly tap and loosen pellets for all 10 or 11×50 mL conical tubes, and then add 4 mL of wash buffer to each loose pellet. Next, with a 5 mL pipette, gently pipette up and down to loosen pellet and then combine volume of all 10 or 11 tubes into 4×50 mL conical tubes (11 mL of wash buffer/cells). To each conical tube add 39 mL of wash buffer. Spin down at 300 g, room temperature, and for 10 minutes. Remove supernatant from all 4×50 mL conical tubes until left with only cell pellets. Lightly tap and loosen pellets for all 4×50 mL conical tubes, and then add 5 mL of wash buffer to each loose pellet. Combine volume of each conical tube into 1×50 mL conical tube (20 mL total volume of cells/infusion buffer). At this point, cells are ready to be counted with Turk's and Trypan blue solutions.

Counting step: Take an aliquot of cells/infusion buffer and place into 2 disposable tubes. To tube 1, add Turk's solution and to tube 2, add Trypan blue solution and mix well. Proceed to counting. Keep the remainder of the cells under the laminar flow hood. After determining the final cell count, adjust cell/infusion concentration to 10×10⁶ cells/mL (expected cell yield is 5-10×108 Cells in 50-100 ml of infusion buffer, respectively). Perform final in-process testing (see FIG. 2: ARMT-SOP-T-001 and -002). If minimum viability from ARMT-SOP-T-002 is obtained, proceed.

At this point, cells are ready to be infused. Proceed to transferring final volume into infusion bags for patient infusion.

*Note: If at any point during the clinical facility protocol cell aggregation/clumping is observed, cell suspensions can be passed through a sterile cell strainer (100 μm—Fisher 22363549) to clarify the product. This is important during the filling of the final cell suspension into syringes and infusion bag. Evidence of aggregation/clumping during final filling may be grounds to discontinue the protocol if no resolutions can be implemented.

### Preparation of Infusion Bag and Infusion

Back load the cells (resuspended in infusion buffer) into a sterile 10 mL syringe. Take the syringe full of cell product and inject into the 100 mL bag of saline using an 18G needle. The 100 mL bag of saline injection is now mixed with cell product. Observe if any clumping/aggregates occurs. Now the Cell product is ready to be handed over to clinician.

Infusion Steps. After mixing the content of the 100 mL saline bag, the bag is spiked on one spike of blood tubing and the 500 mL bag of saline is spiked on the other. The blood tubing is primed with the 500 mL bag of saline. The Y tubing to the 500 mL saline bag is clamped once the tubing is primed. The blood tubing is then connected to the lowest Y tubing site of the patients IV tubing and the 100 mL bag of saline+cell product is infused over 20 minutes. Once the 100 mL bag is empty, it is gravity fed by 500 mL bag to clear bag and tubing (flushing all cells out of 100 mL bag and Y tubing); this is allowed to infuse into patient. Once flushed and emptied the Y tubing to the 100 mL bag is clamped. Then the Y tubing to the 500 mL bag is opened and approximately 150 to 200 mL of pure saline is flushed to clear lines and make sure all cells are infused.

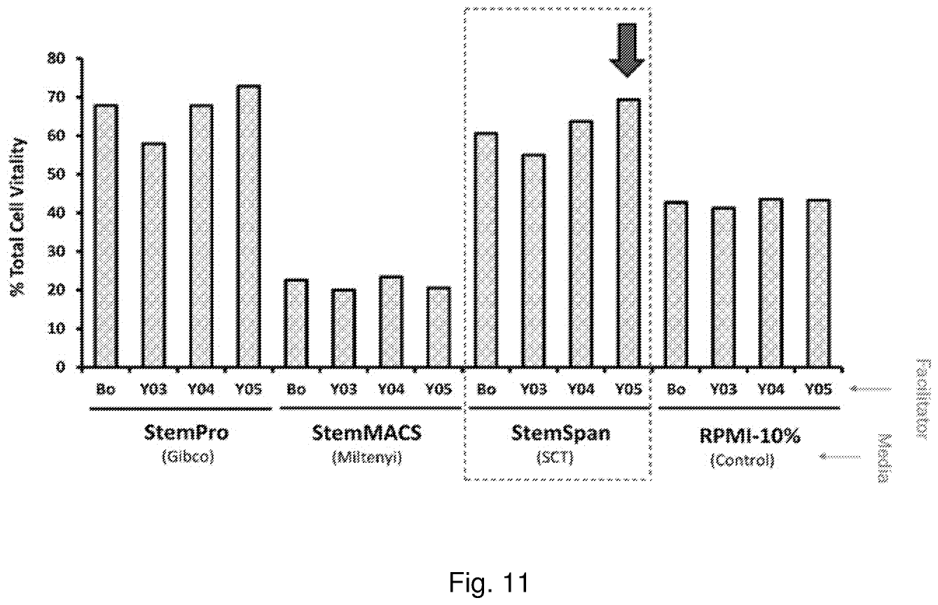

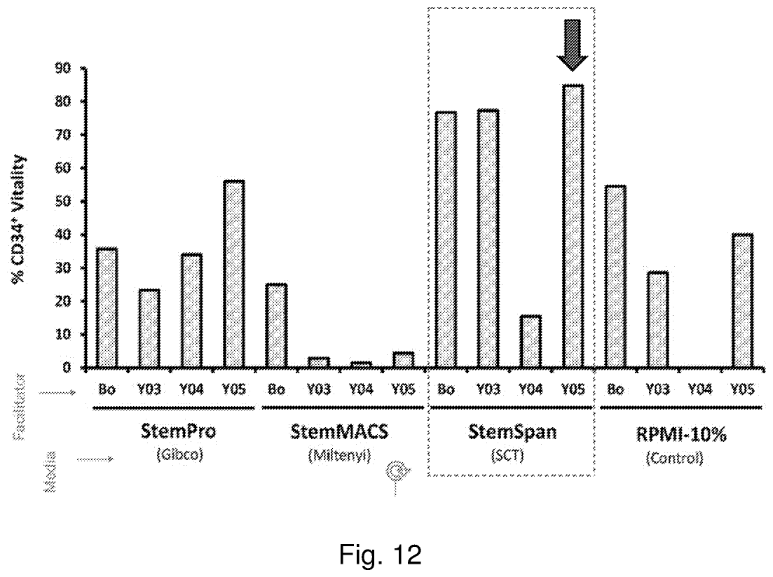

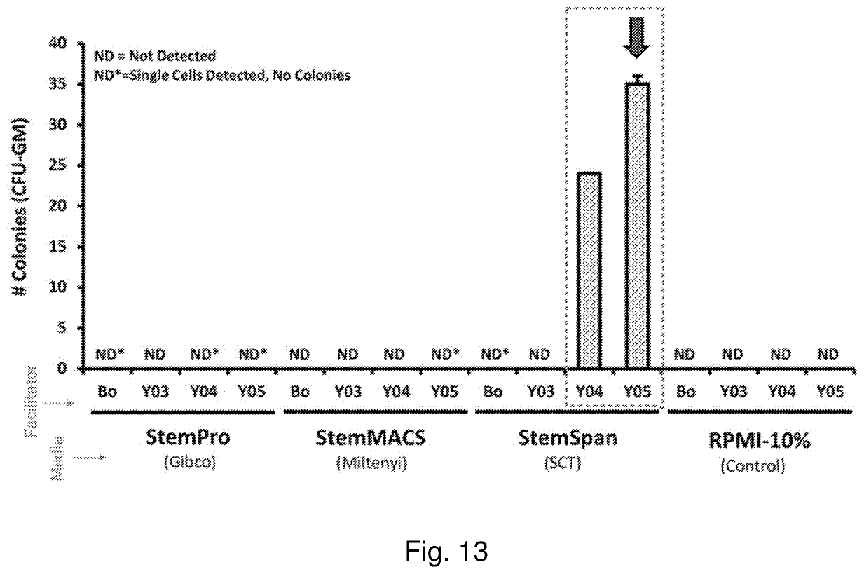

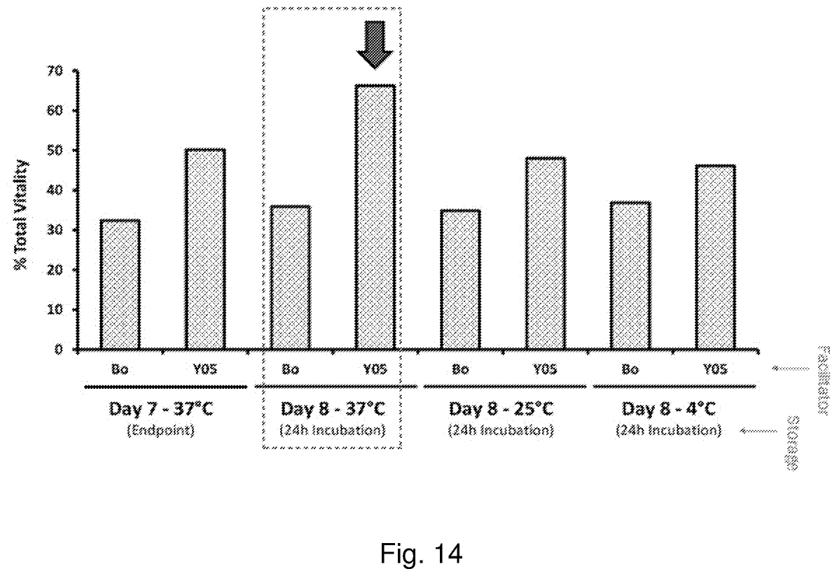

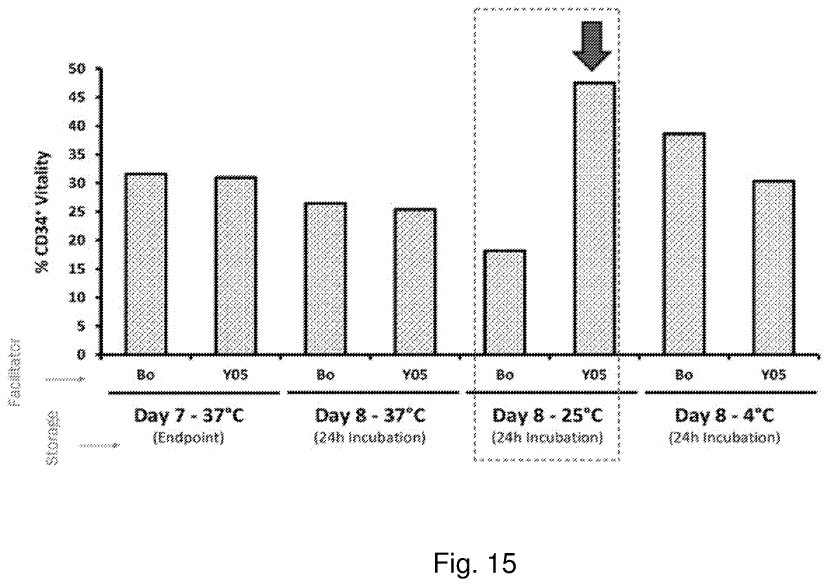

FIG. 11 shows data demonstrating superior total cell vitality is preserved with the animal-free media, StemSpan and StemPro from Stem Cell Technologies and Gibco, respectively. Young donor ‘Y05’ demonstrates superior restoration of aged donor Bo compared to the other young donors. FIG. 12 shows data demonstrating superior CD34⁺ cell vitality is preserved with the animal-free media, StemSpan from Stem Cell Technologies. Young donor ‘Y05’ demonstrates superior restoration of aged donor Bo compared to the other young donors. FIG. 13 shows data from the clonogenic assay confirms that maximal restoration of Bo stem cell function is obtained when donor Y05 is utilized as a facilitator in StemSpan media. FIG. 14 shows data demonstrating enhanced total cell health of restored Bo cells 24 h post-restoration under 37° C. conditions. Findings from the stem cell health study (FIG. 15) will elucidate whether 25° C. or 37° C. is optimal for post-restoration stability during sample shipment. FIG. 15 shows data demonstrating enhanced stem cell health of restored Bo cells 24 h post-restoration under 25° C. conditions, however total cell health appears to be enhanced at 37° C. (FIG. 14). While both of these temperatures yield comparable results, extended sample storage at 37° C. in the absence of 5% C₀₂ is generally frowned upon in the hematopoietic transplantation field. For this reason, samples post-restoration will be shipped at ambient temperature.

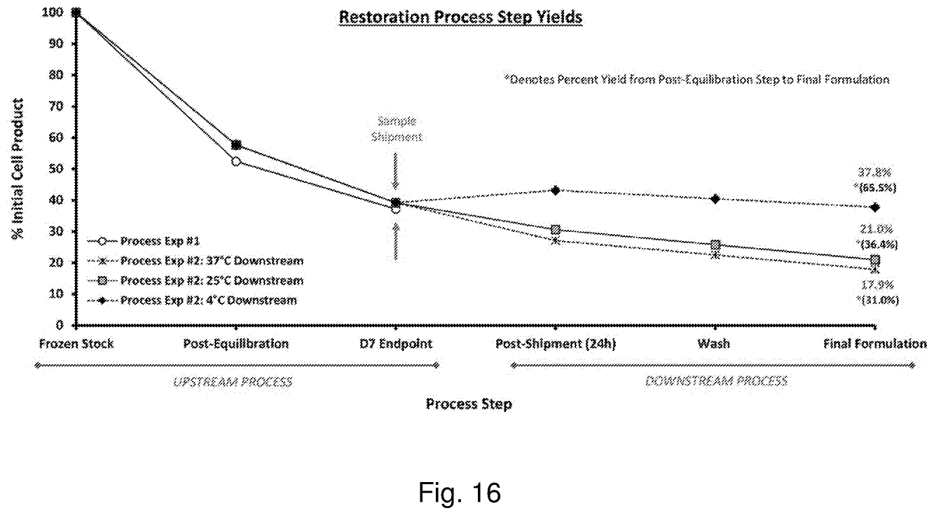

FIG. 16 shows recovery of restored B_{O} cells from time of transwell seeding (denoted by *) is approximately 31-37% for the 2 viable temperature conditions (37° C. and 25° C., respectively). The initial 50% loss post-thaw is likely related to the process by which B_{O} cells were mobilized/collected and/or cryopreserved/stored. Typical initial post-thaw, step-loss for Advanced Regen mobilization/harvest/cryopreservation protocol is <25%.

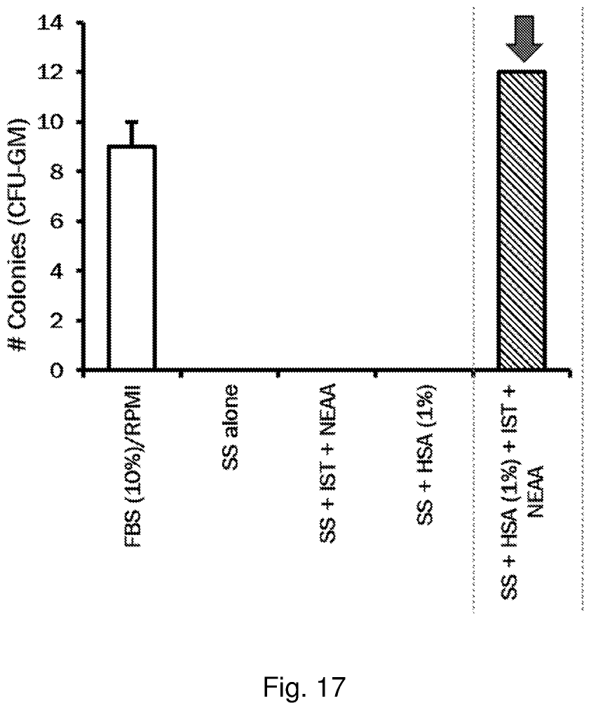

FIG. 17 shows the evaluation of Young Donors' Ability to Restore Aged Donors in Xenogenic-Free Restoration Media Stem Span (SS) supplemented with MEM Non-Essential Amino Acids Solution (NEAA), Insulin-Transferrin-Selenium (IST)-and H.S.A. FIG. 17 provides data from the clonogenic assay confirms that maximal restoration of stem cell function is obtained when donor Y04 is utilized as a facilitator in StemSpan media supplemented with H.S.A., IST and NEAA.

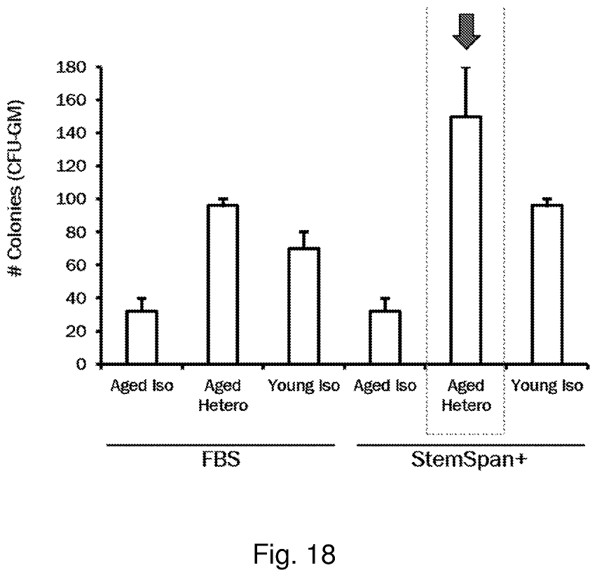

FIG. 18 shows the evaluation of Young Donors' Ability to Restore Aged Donors LC and RC in Xenogenic-Free Restoration Media supplemented with MEM Non-Essential Amino Acids Solution, Insulin-Transferrin-Selenium-Sodium Pyruvate and H.S.A. FIG. 18 provides data from the clonogenic assay confirms that maximal restoration of stem cell function is obtained when the young donor is utilized as a facilitator in Stem Span media supplemented with H.S.A., IST and NEAA and Sodium Pyruvate.

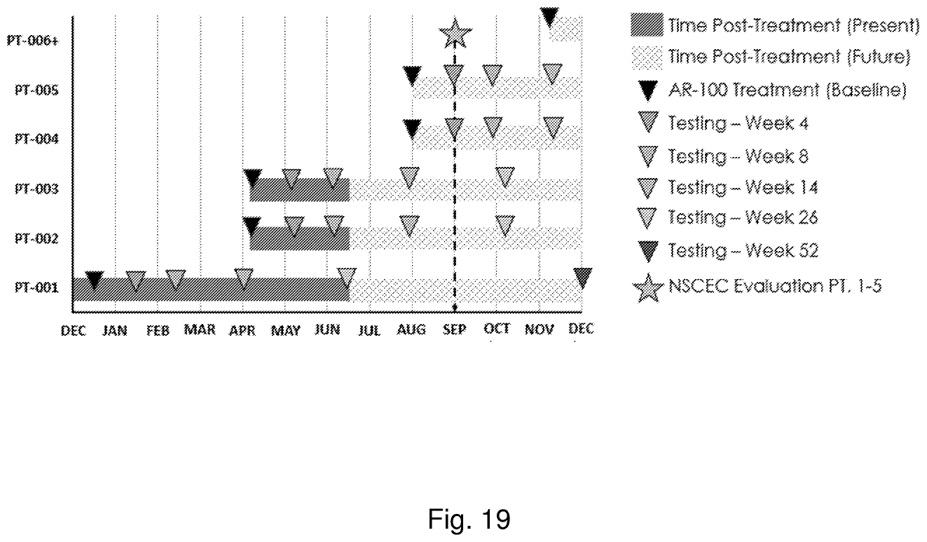

FIG. 19 shows potential testing regimes for patients who have underwent treatment.

### Example 5

Collections of mobilized mononuclear cell samples from healthy aged and young donors are performed at the qualified study site in the United States. Participants are given an FDA approved, hematopoietic mobilizing agent on a daily basis at the currently recommended dosages. In some embodiments, participants will be given Filgrastim/Neupogen® (G-CSF) at 5-10 ug/kg by subcutaneous injection daily for 5 consecutive days. G-CSF stimulates the bone marrow to produce a large number of hematopoietic and progenitor stem cells and mobilizes them into the peripheral blood stream. CBCs to assess the response to the mobilizing agent will be performed prior to mobilization and on the final day of mobilization prior to mononuclear cell (MNC) collection. On the 6th day, mobilized peripheral blood MNCs will be collected by leukapheresis using a cell separator. Leukapheresis will be performed according to the manufacturer's instructions to process 18 L of blood at a flow rate of 50 to 100 mL per min. Mobilized MNC collections generally require 4 to 6 hours for completion. Participants will generally have only one MNC collection performed immediately following mobilization. The product of 1 full MNC collection is referred to as a Leukopak. For cell restoration therapies utilizing freshly harvested cells, aged and young donor collections should be coordinated to occur within 24 hours of each other. Fresh leukopaks should be processed within 24 hours of collection and should be stored at room temperature.

In FIG. 5, the steps for cellular restoration and the sites at which they are performed: the first 5 steps are at a laboratory site followed by the last 3 steps at a clinical site. Typical number of MNCs harvested from the leukapheresis procedure range from 25-50×10⁹ cells, with viability >95% and a collection volume of 300-400 mL (approximately 100×10⁶ cells/mL). Prior to cell processing, a sample of the Leukopak should be collected and cell number determined by counting with a hemocytometer. Further, cell viability should be determined using Turk's solution. Additional evaluation of expression for the biomarkers CD45 and CD34 in the MNC collection can be made by flow cytometry to determine the percentage of leukocytes and hematopoietic stem/progenitor cells, respectively. Typical number of CD34+ cells collected from mobilized leukapheresis range from 1-2×107 cells per harvest dependent on the age of the donor, with young donors demonstrating greater yield. The cell restoration protocol requires an initial transwell seeding dose of 2×109 cells, thus a large excess of MNCs are left from the collection. Cells not utilized for the initial procedure are cryopreserved for potential utilization in secondary treatments. Cells are diluted in cryopreservation media at a 1:1 ratio to yield a final cell suspension of approximately 50×10⁶ cells/mL containing human serum albumin (HSA) and DMSO. Cells are then frozen using a programmable controlled rate freezer at a rate of −1° C./min to a temperature of −100° C. for transfer to liquid nitrogen storage.

Transwell Restoration 3×10⁹ MNCs from each of the aged and young donor Leukopaks are pelleted by centrifugation for 10 min at 300 g and then resuspended in an equal volume of StemSpan ACF cultivation medium (Stem Cell Technologies). Cell suspensions are counted and 2×10⁹ aged and young MNCs are seeded into transwell cultures, with the young cells in the upper chamber and aged cells in the lower chamber. Cells are then incubated at 37° C. and 5% CO2 for 7 days to allow for cell restoration. On the 4th day of culture, approximately 15% of the total culture media per transwell is replenished with fresh cultivation medium.

On the 7th day of transwell culture the partially processed cell preparation will be transferred to a clinic for harvesting, removal of cellular debris and dead cells, and purification of the aged restored cells from the transwell system. The aged restored cells are collected and pelleted by centrifugation for 10 min at 300 g. Typical cell loss during the restoration period is approximately 50%. Pelleted cells should be resuspended in an equal volume of wash/infusion buffer (HSA in saline) and an aliquot removed for cell counting and viability assessment.

Cells are pelleted again by centrifugation for 10 min at 300 g and resuspended in wash/infusion buffer at a concentration of 1×10⁷ viable cells/mL. This procedure should result in an approximate dose of 1×10⁹ restored cells (approximately 100 mL) for patient infusion. This initial dose was extrapolated from a similar efficacious dosing scheme in pre-clinical studies. Cells are then transferred to a 500 cc infusion bag and infused to the patient intravenously.

In some embodiments, the source of aged stem cells will be from adipose tissue.

### Assessment of Clinical Safety & Efficacy

Procedure: Blood collected from the patient prior to the procedure and at 2, 6, 12 and 24 months after the procedure will be used to measure: (1) percentages of immune cell populations, (2) CBC profile and (3) immune function/response. This determination will be made by flow cytometry utilizing antibodies specific for the immune cell types. Table 5 below describes the specific populations of interest and the cell identification marker.

TABLE 5
Immune Cell Phenotyping

| Cell Marker for Antibody Labeling | Immune Cell Type |
|---|---|
| CD45 | Leukocytes |
| CD3, CD4, CD8, CD19 | Lymphoid |
| CD33, CD14 | Myeloid Cells |
| CD3, CD56 | Natural Killer Cells |
| CD25 | Activated T cells |

Determination of Efficacy: As the immune system ages, the ratio of myeloid to lymphoid cells in the blood increases while the ratio of T Helper (CD4) to T Cytotoxic (CD8) T cells decreases. These cell ratio metrics will be utilized to determine clinical efficacy after treatment.

The myeloid to lymphoid ratio will be determined by the formula: % CD33/(% CD3+% CD19) The T Helper to T Cytotoxic ratio will be determine by the formula: % CD4/% CD8 Outcome: Aged individuals that receive the restoration procedure see a decrease in blood myeloid to lymphoid ratio as well as an increase in the T Helper to T Cytotoxic ratio within 3 months following the procedure.

2. CBC Analysis Outcome: None unrelated to the phenotypic results.

3. Functional Analyses Determination of Efficacy: As the immune system ages, the ability to mount an immune response following a stimulus is diminished. To determine whether the restoration procedure is efficacious, immune function is determined by the ability to respond to pan-T-cell mitogen (Phytohemagglutin); antigens (T. toxoid, C. albicans); B-cell mitogen (Pokeweed or lipopolysaccaride) and T-cell specific antibody (anti-CD3). MNCs are isolated from periphal blood of the patient as well as age-matched and young controls by Ficoll-Hypaque gradient and 2×10⁶ cells/mL are stimulated with PHA (1/100) or Pokeweed (25 μg/mL) or antigens (C. albicans at 1/40 final dilution); T. toxoid at 1/50 or anti-CD3 at 1 anti-CD3 coupled bead: 2 cells). 200 μL of each cell suspension is added to 96-well flat-bottom plates in triplicates and then incubated at 37° C. Cultures with mitogens and antigens were pulsed with 1 μM of tritiated thymidine (3HTdR) at day and 5, respectively. At 16 h after pulsing, the cells are harvested and then studied for 3HTdR using a cell harvester.

Outcome: Aged individuals that receive the restoration procedure see an increase in immune proliferation in response to stimulation with anti-CD3, T- and B-cell mitogens, and antigens. This increase will be determined by cross-sectional comparison to age-matched and young controls. The data will also be compared longitudinally to pre-treatment baseline response for the patient.

4. Secondary Treatments

Evaluation of patient response to treatment will be made a 2, 6, 12 and 24 months after initial infusion. Patients who respond to treatment and see improved immune function during follow-up will be re-assessed at 12 and 24 months post-treatment to determine restoration stability or regression. If efficacy measures begin to regress towards baseline levels, secondary procedures utilizing the previously cryopreserved cells should be discussed with the patient. A similar guideline should be followed for non-responders at the 12-month follow-up to determine whether a secondary treatment at a higher cell dose is of interest.

5. Clinical Status

The primary end-point for the 5 patients enrolled in this study is safety at 2 months after treatment. As an initial measure of efficacy, the SF-36 quality of life instrument is used. RAND, as part of the Medical Outcomes Study (MOS), a multi-year, multi-site study to explain variations in patient outcomes, developed the 36-Item Short Form Health Survey (SF-36). SF-36 is a set of generic, coherent, and easily administered quality-of-life measures. These measures rely upon patient self-reporting. The SF-36 is now widely utilized by managed care organizations, research groups and by Medicare for routine monitoring and assessment of care outcomes and defining efficacy of novel treatment strategies in adult patients. The SF-36 is used to assess quality of life in each patient at baseline, and 3, 6 and 12 months after treatment.

Safety Considerations

There is no evidence of tumorgenesis, oncogenesis, or enhanced tumor growth. To directly extend the evidence of safety, as shown in FIG. 1, a humanized mouse study was performed. In this study, mice were humanized with stem cells from aged donor patients. Following verification of stem cell engraftment, these animals were infused with autologous control or restored stem cells. This protocol parallels that for patients. Fourteen weeks later these immune function, aging gene expression and senescence programming in those animals infused with restored cells was much more similar to that seen in young animals, compared to those animals that received control cells. The reversal of senescence is a key to the reversal of the effects on aging of stem cells. Importantly, the organs from animals that received control and restored stem cells were harvested and reviewed by an independent pathologist. There were no findings of any evidence of pathology in these animals.

The evidence demonstrates that these approaches do not result in any harm to the recipient. This conclusion as it relates to the protocol is further supported and confirmed by our demonstration of safety using humanized mice.

### Example 6

Patient testing before and after treatment may include one or more of the following: Cellular Biomarkers and Safety—Quest Diagnostic; Myeloid leukemia panel (pathologist review); Myeloid/Lymphoid ratio; Lymphocyte proliferative response (Mitogen- and Antigen-based); Natural Killer cytotoxicity assay; CBC; Biochemical & Genetic Biomarkers—Rutgers/Houston Labs; Senescence and aging gene array (blood mononuclear cells); Senescence protein array (blood plasma); SF-36 Quality of Life Survey—Self Administered; Set of generic, coherent, and easily administered quality-of life measures.

### Example 7: MicroRNAs Improve Function of Aged and Defective Biological Systems

### Overview

Blood of healthy subjects (e.g., the blood of subjects that are younger or are generally healthier than particular patients) may compartmentalize pleiotropic factors that prevent age-associated tissue dysfunction or other dysfunction. The diminishing function of the hematopoietic and immune systems, for example, throughout an organism's lifetime as the organism ages, leads to compensatory increases in immune-related diseases, including cancer. While these dysfunctional processes can occur in the young and aged alike, for the purposes of this study, healthy aged individuals and young healthy individuals were used to determine the efficacy of using pleiotropic factors to improve dysfunctional cells. To this end, a heterochronic culture model was developed to target the declining function of hematopoietic stem (HSCs) and progenitor cells (HPCs) in aged patients, which allowed the study of various therapeutic factors (e.g., restorative factors) released from young blood cells. It was postulated that young adults (18-29 y/o) with healthy lifestyles could yield robust blood samples to identify factors that could restore and/or act as a therapeutic to aged or dysfunctional tissues and cells. To evaluate impairments related to, in this instance age, without co-morbidities, healthy aged donors (>60 y/o) were recruited. In other instances it is envisioned that the techniques disclosed herein could be used more generally with patients of any age that suffer dysfunction in part based on cellular factors. In this study, enrichment of HSCs, HPCs and stroma in donor blood samples was performed by granulocyte-colony stimulating factor (G-CSF)-mobilization (though other mobilization techniques are envisioned). Heterochronic cultures were established in a transwell system that allowed for exchange of soluble factors but no direct interaction of younger aged cells and aged cells. It was found that young paracrine factors induced increases in aged stem and progenitor clonogenicity, T cell activity and cell-mediated cytotoxicity. These findings were validated in humanized mice engrafted with an aged lymphohematopoietic system. Exploration of the restorative and/or therapeutic mechanism revealed a causal role for downstream targets in aged cells, PAX 5 and PPM1F and the interaction thereof with miRNAs from, for example, exosomes of younger subjects, including but not limited to miR-619-5p, miR-1303 and miR-4497. It was also determined that these systems could be targeted directly with one or more of these miRNAs to induce restoration without the need for heterochronic culture. Thus, in some embodiments, exploitation of miRNAs can provide an immunotherapy and/or therapeutic mechanism to restore and/or repair dysfunction in the patients (e.g., restoring or repairing an aging body's endogenous immune system and/or restoring or repairing a body's endogenous immune system where that system is dysfunctional). This therapeutic system was also demonstrated to have potential benefit in treating and/or preventing cancer.

### Introduction

Aging is a highly complex biological process and the leading risk factor for the chronic diseases that account for the bulk of morbidity, mortality and health costs. Regrettably, there is no evidence to suggest that aging might be controlled by a single, hidden “master switch”. Rather, the complexity of organismal aging is driven by cellular dysfunction at the macromolecular and/or organelle level, which ultimately leads to a decline in tissue function and the manifestation of disease.

As cells age (or otherwise become dysfunctional) they undergo epigenetic alterations that lead to dynamic changes in gene expression and increased likelihood of oncogenesis and cellular transformation. Cell entry into the non-proliferative, yet metabolically active, state of senescence serves a protective role to avert transformation. The frequency of senescent cells in the body increases as organisms age. Senescent cells exhibit a unique profile of enhanced secretory factor production, termed the senescent-associated secretory phenotype (SASP). Many of these factors are pro-inflammatory and/or tumor-supportive, thus cellular senescence is a fundamental aging mechanism tied to the progressive breakdown of tissue function with age. A new class of small molecule drugs, termed senolytics, may directly target senescent cells and lead to significant health span extension in mice. Lifespan extension in transgenic mice that selectively expressed suicide genes in senescent cells can also be accomplished. Physiologically, lifespan-extending effects may be observed for miRNAs, such as miR-17, that inhibit senescence signaling.

Interventions that attempt restoration of aging tissues have been researched for many years, often utilizing younger tissues as the source of restoration. This age-old concept has been documented as early as the 19th century in the form of animal graft experimentation. The first grafting experiments to study heterochronic parabiosis, which is the surgical suturing of two animals of different ages to enable development of a single, shared circulatory system between young and old, were carried out in the late 1950s and early 1960s. Several recent studies have revived the experimental procedure to demonstrate that the young circulatory system contains restorative factors that can rejuvenate aged tissues and cells. Further, other groups expanded on the heterochronic parabiosis model to show a role for systemic factors in the circulation of young parabionts that induce rejuvenation of cognitive, cardiac and skeletal muscle of the matched aged animal. While these factors were able to restore select tissue function, the infusion of young plasma and/or factors therefrom into aged animals have not been demonstrated to increase longevity, nor has its effects on the aged lymphohematopoietic system been explored.

Diminishing function of the aging lymphohematopoietic system leads to compensatory increases in immune-related diseases, including cancer. This system critically depends on adult hematopoietic stem cells (HSCs) throughout an organism's lifetime to generate progenitor cells and mature effector blood cells. The decline of HSCs and the adaptive immune response is a major source of morbidity and mortality, as decreased immune surveillance leads to increased incidence of cancer, infectious disease and immune-related disorders. Described herein is an approach to restore and/or repair the aging lymphohematopoietic system. The approach utilizes a cell culture adaptation of the heterochronic model to harness restorative factors from young blood that promote aged HSC function and reduce immune senescence.

### Materials and Methods

### Stem Cell Mobilization and Leukapheresis

Healthy aged (>60 y/o) and young (18-29 y/o) individuals were recruited according to specified inclusion and exclusion criteria (Table 4), with qualified donors enrolled for stem cell mobilization and leukapheresis. All recruitment, mobilization and leukapheresis were conducted under an approved IRB and informed consents by HemaCare Corporation (Van Nuys, Calif.), an FDA-registered, AABB-accredited collection center operating under GMP-compliant, validated procedures and equipment. Study participants were dosed subcutaneously with Neupogen® (G-CSF) at 5 μg/kg/day for 5 days to stimulate the bone marrow (BM) to expand the HSC/HPC compartment and mobilize it into the peripheral blood stream. On the 6th day, mobilized peripheral blood (MPB) was collected with the Spectra Optia® Apheresis System using continuous flow centrifugal technology directly into the collection bag. Leukapheresis was performed according to the manufacturer's instructions to process 18 L of blood at a flow rate of 50 to 100 mL per min. Participants had only one mononuclear cells (MNC) collection performed immediately following mobilization. The product of 1 full MNC collection is referred to herein as a Leukopak. Mobilized MNC collections generally required 4 to 6 hours for completion, at which time cells were shipped to Rutgers for processing and biobanking. The research use of these cells followed a protocol approved by the institutional review board (IRB) of Rutgers Biomedical Health Sciences.

### Umbilical Cord Blood Collection

Umbilical cord blood (UCB) collected from mothers delivering at participating hospitals was obtained by Community Blood Services (Montvale, N.J.), an AABB-accredited blood bank registered with the FDA. Individual collections of less than 100 mL were designated for research use, and donated under a protocol approved by the IRB of Rutgers. 10 whole UCB units were obtained and processed within 24 h of collection by Ficoll-Hypaque (Sigma) density gradient to isolate the mononuclear fraction and remove red blood cells and granulocytes. MNCs were cryopreserved for later use in transwell cultures.

### Isolation of CD34+ Cells and Cryopreservation of Blood Products

Immediately following collection, mobilized Leukopaks (400 mL) were split into 2 bags (200 mL each) and shipped overnight at 4° C. for subsequent processing and biobanking. All samples were processed within 16 h of donor leukapheresis. Total nucleated cells (TNCs) from the first collection bag were pelleted at 4° C. for 10 min, washed with cold buffer containing 2% human serum albumin (HSA; Irvine Scientific) and then resuspended in the original 200 mL supernatant. Chilled cryopreservation media with 3.6% HSA and 20% DMSO was added dropwise to the TNC suspension at a 1:1 ratio while gently shaking, for a final TNC concentration of approximately 50×106 cells/mL. The second half of the collection was used to purify CD34+ cells. TNCs were pelleted at 4° C. for 10 min and then resuspended in cold MACS buffer (0.5% bovine serum albumin, 2 mM EDTA in PBS, pH 7.2) at a concentration of 108 cells per 300 μL buffer. Cells were incubated in 100 μL each of FcR Blocking Reagent and CD34 MicroBeads (Miltenyi Biotec) per 10⁸ total TNCs for 30 min at 4° C., then washed in MACS buffer. Magnetic separation was performed by positive selection with LS columns (Miltenyi), and purified CD34+ cells immediately resuspended in cold cryopreservation media (80% FBS, 10% RPMI-1640 and 10% DMSO). Similar media was used for MNCs isolated from UCB. All cells were frozen using a controlled rate freezer at a rate of −1° C./min until at temperature of −100° C. was reached, at which time vials were transferred into liquid nitrogen for long term storage.

### Heterochronic Transwell Culture

TNC vials were quickly thawed at 37° C. and cells added dropwise 1:10 to pre-warmed RPMI-1640 equilibration solution containing 5% HSA, 30 U/mL DNase I (Sigma), 5 mM MgCl2 and 5 mM CaCl₂), and incubated at 37° C. for 3-4 hrs. After equilibration, cells were pelleted, washed in RPMI-1640 with 2% BSA and resuspended in complete media containing 10% FBS. Cells were seeded at densities of 10 or 30×106 cells/well in 12- or 6-well plates, respectively, containing 0.4 m transwell inserts (BD Falcon). Heterochronic cultures were established with young cells in the upper chamber and aged cells in the lower chamber. Cells were incubated at 37° C. for 7 days, with 15% of culture media replenished with fresh media on the 4th day. On the 7th day, cells were harvested by centrifugation and evaluated.

### Cell Phenotyping by Flow Cytometry

Cell surface staining was performed by incubation with lineage-specific antibodies. Briefly, 106 cells were resuspended in PBS and incubated with appropriate antibodies for 30 min at RT in the dark. Antibody dilutions were performed according to manufacturer's recommendations. Isotype IgGs were used as controls. Cells were washed with PBS and acquired on a FACSCalibur flow cytometer (BD Biosciences). Data were analyzed using BD CellQuest Pro™ software (BD Biosciences). All antibodies were purchased from BD Biosciences: CD3-APC (UCHT1), CD3-PerCP-Cy5.5 (UCHT1), CD4-PE (RPA-T4), CD8-APC (RPA-T8), CD19-PE (HIB19), CD25-PerCP-Cy5.5 (M-A25), CD33-APC (WM53), CD34-APC (581), CD38-FITC (HIT2), CD45-PerCP-Cy5.5 (HI30), CD45-FITC (HI30), CD45-PE (HI30), CD56-PE (B159) and HLA-DR-PE (G46-6). In some embodiments, one or more of these antibodies is used in methods of preparing target cells as disclosed herein.

### Modified Long-Term Culture-Initiating Cell Assay

A good in vitro system to study long-term HSC function is the long-term culture-initiating cell assay (LTC-IC). Briefly, LTC-IC assays were initiated by seeding an aliquot of cells from heterochronic or isochronic culture onto irradiated (1,500 cGy) BM stromal cells (3×104/cm2) that had been previously sub-cultured. Beginning at week 6, 103 non-adherent cells were assayed every 2 weeks, up to week 12, for primitive hematopoietic progenitors in clonogenic assays (as discussed in further detail elsewhere herein).

### Clonogenic Assay

Non-adherent cells from LTC-IC assays, as well as cells from isochronic and heterochronic cultures, were studied for progenitors in short-term methylcellulose culture using a CFU-GM readout. Briefly, cultures were initiated by seeding 150 cells/mm² in clonogenic media containing 3 U/mL of rhGM-CSF (R&D Systems). After 10 days, cultures were scored by a single blinded observer, and colonies enumerated for CFU-GM.

### Cell Vitality and Mitosox Assays

106 TNCs were labeled with anti-CD34-APC and -CD45-PerCp-Cy5.5. For Cell Vitality assay, cells were washed and co-stained with 10 nM Sytox and 200 nM C12-resazurin (Molecular Probes) in 100 μl volume. Cells were incubated for 15 min at 37° C., then diluted 5 times with PBS. For Mitosox assay, cells were washed and incubated with 5 M MitoSox™ Red (Molecular Probes) for 10 min at 37° C. in the dark and then washed again with warm HBSS/Ca/Mg buffer. A FACSCalibur flow cytometer was used for data acquisition in both assays.

### Cytotoxicity Assay

Cell cytotoxicity was determined with the CFSE/7-AAD Cell Cytotoxicity Kit (Cayman Chemical) according to manufacturer's specified instructions. Briefly, the human chronic myelogenous leukemia cell line, K562, (ATCC #CCL-243) was used as target cells. 107 K562 cells were labeled for 15 min with CFSE dye, washed twice and diluted 100-fold for 30 min incubation at 37° C. Cells from 7-day isochronic or heterochronic cultures were used as effectors. Effector and target cells were added to 6-well plates at the following effector-to-target (E:T) ratios: 0:1, 6.25:1, 12.5:1 and 25:1, and cell mixtures incubated for 4 h at 37° C. Cells were harvested and counter-stained with 7-AAD. 50,000 events were acquired on the FACSCalibur flow cytometer, and data analyzed using BD CellQuest Pro™ software. Target cells incubated alone with 7-AAD served as control to calculate spontaneous lysis, while effector cells alone with 7-AAD served as control to detect dead effector cells. Percent lysis was calculated according the following formula: [(cells positive for both CFSE and 7-AAD/total CFSE-labeled cells)*100−spontaneous % lysis].

### T Cell Activation Assay

T cell activation was determined using the T Cell Activation/Expansion Kit (Miltenyi). Briefly, anti-biotin MACSiBead Particles were loaded with CD2, CD3, and CD28 antibodies. Cells from 7-day isochronic or heterochronic cultures, or MNCs isolated from huNSG mouse blood by Ficoll-Hypaque density gradient, were incubated with loaded anti-biotin MACSiBead Particles at a 1:2 bead to cell ratio for 72 h to activate T cells. Addition of unloaded MACSiBead Particles served as negative control. After 72 h, cells were fluorescently labeled using CD45-FITC, CD4-PE, CD25-PerCP-Cy5.5 and CD8-APC to determine T cell activation status by flow cytometry.

### Mixed Lymphocyte Reaction

One-way mixed lymphocyte reaction (MLR) was performed. Briefly, cells from 7-day isochronic or heterochronic cultures were seeded in 96-well, flat-bottom plates (Corning) and equal volumes (0.1 ml) of stimulators (gamma-irradiated, freshly thawed aged cells) and responders (aged cells from heterochronic or isochronic culture) added to each well in triplicate. Thawed aged cells incubated with cells from young isochronic culture served as positive control. Cultures were pulsed with 1 μCi/well of [methyl-3H]TdR (70-90 Ci/mmol; NEN) during the last 16 h of a 4-day culture. Cells were harvested with a PhD cell harvester (Cambridge Technologies) onto glass-fiber filters, and [3H]TdR incorporation quantified in a scintillation counter (Beckman Coulter). Results were expressed as the stimulation index (S.I.), which is the mean dpm of experimental cultures (responders+gamma-irradiated stimulators)/dpm of responder cells with only medium.

### Humanization of NSG Mice and Adoptive Transplant

5-week old, female NOD/scid IL2Rγnull (NSG) mice were purchased from Jackson Labs and housed in an AALAC-accredited facility at Rutgers, New Jersey Medical School (Newark, N.J.). The protocol was approved by the Institutional Animal Care and Use Committee, Rutgers School of Biomedical Health Sciences (Newark, N.J.). Mice were acclimated in the Rutgers barrier animal facility for 1 week prior to experimental use. 6-week old mice were subjected to 150 cGy whole body gamma irradiation using a Mark-I cesium irradiator unit. 5 hours post-irradiation, mice were injected i.v. with 5×10⁵ human CD34+ cells isolated from aged or young study donor Leukopaks. Engraftment proceeded over 15-19 weeks, with peripheral blood chimerism monitored at 9 and 13 weeks post-transplant. % Chimerism was detected by co-labeling blood with anti-human CD45-APC (HI30) and anti-mouse CD45-FITC (30-F11) for monitoring by flow cytometry. Humanized NSG (huNSG) enrolled in the treatment arm of the transwell-based animal study were given a 2nd i.v. injection of 5×10⁵ autologous aged cells from 7-day isochronic (non-restored) or heterochronic (restored) cultures that were CD3-depleted (Miltenyi) prior to transplant, or saline control. The miRNA-based animal study was performed as above, except that the treatment arm utilized autologous aged cells transfected with 60 nmol total of either miR-619 alone, miR-combo (miR-619, -1303, 4497) or control RNA. Transfections utilized the HiPerFect reagent (Qiagen), and cells were transfected for a total of 7 days prior to CD3 depletion and i.v. injection as above. Mice were sacrificed at 14-15 weeks post-transplant, and blood, BM and spleen harvested for biochemical, phenotypic and functional analyses. Major organs were also harvested for histological assessment. Tissue embedding, processing and staining were performed by the Digital Imaging and Histology Core of Rutgers-New Jersey Medical School Cancer Center (Newark, N.J.). Histologic findings were confirmed on H&E slides by a board-certified veterinary pathologist.

### Senescence Protein Array and T Cell Cytokine Array

Detection of senescence associated secretory factors (SASFs) in plasma of huNSG mice was performed using Custom C-Series Human Antibody Arrays (Ray Biotech). Arrays were labeled with antibodies to 68 different factors linked to cellular senescence. Briefly, blood from huNSG mice was pelleted for 10 min at 300 g and plasma supernatant collected in siliconized microfuge tubes for SASF determination. Incubation and detection of factors within plasma followed the manufacturer's suggested protocol. Background levels were calculated by incubating the arrays with plasma from non-humanized NSG mice and then subtracting the obtained values from each huNSG sample.

For the T Cell Cytokine Array (Ray Biotech), conditioned media was collected from cells isolated from huNSG peripheral blood that were stimulated as per the T cell activation assay (see above) after 72 h and stored in protein lo-bind tubes. The protocol followed manufacturer's suggested recommendations. For both the custom SASF and T cell cytokine arrays, densitometry was performed using the UN-SCAN-IT densitometry software (Silk Scientific). Hierarchical clustering and heat map generation were performed with Heatmapper software.

### Senescence and Aging Gene Arrays

BM from huNSG were flushed with a 26-gauge needle and collected for subsequent purification of engrafted human cells with the Mouse Cell Depletion Kit (Miltenyi). Total RNA (2 μg) was extracted from purified cells using the RNeasy Mini Kit (Qiagen) and reverse-transcribed with the RT2 First Strand Kit (Qiagen). 20 ng of cDNA was used for qPCR with the Human Cellular Senescence and Human Aging RT² Profiler™ PCR Arrays (Qiagen). Arrays were run on the 7300 Real Time PCR System (Life Technologies) with the cycling profile (40 cycles): 94° C. for 15 seconds and 60° C. for 45 seconds. Gene expression analysis was performed using Qiagen PCR Array Data Analysis Software and normalized to five housekeeping genes provided within each array. Hierarchical clustering and heat map generation were performed with Heatmapper software.

### Exosome Isolation and Nanoparticle Tracking Analysis

Exosomes were isolated from cell culture media by the Total Exosome Isolation Kit (Life Technologies), using a modified version of the manufacturer's protocol. Briefly, 7-day isochronic and heterochronic cultures were established with Exosome-depleted FBS Media Supplement (System Biosciences), and culture media collected on the 4th and 7th days. Cells were pelleted and supernatant transferred to another tube for further clarification at 2000 g for 30 min to remove residual cells and debris. The remaining supernatant was transferred to a fresh tube and 0.5 volumes of Total Exosome Isolation reagent added for overnight incubation at 4° C. The following day samples were centrifuged at 10,000 g for 1 hr at 4° C. to pellet the exosomes for subsequent nanoparticle tracking analysis (NTA) or long-term storage at −80° C. Analysis of absolute size distribution of exosomes was performed using the NanoSight LM10 with NTA3.1 software (Malvern). Particles were automatically tracked and sized based on Brownian motion and the diffusion coefficient. For NTA, exosomes were re-suspended in 0.5 mL of PBS and measured using the following parameters: Temperature=25.6+/−0.5° C.; Viscosity=(Water) 0.99+/−0.01 cP; Measurement time=30 see; Syringe pump speed=30. The detection threshold was similar in all samples. Three recordings were performed for each sample.

miRNA Profiling by NGS

Total RNA from exosomes and cells was isolated using the miRCURY RNA Isolation Kit (Exiqon) with small and large RNAs fractionated with the RNeasy MinElute Cleanup Kit (Qiagen), both according to manufacturer's recommended specifications. Half of the small RNA fraction (200 ng) was used in library preparation with the NEBNext Multiplex Small RNA Sample Prep Set for Illumina—Set 1 (NEB), according to the following protocol: (1) ligation of the 3′ SR Adaptor, (2) hybridization of the reverse transcription primer, (3) ligation of the 5′ SR Adaptor, (4) reverse transcription for first strand cDNA synthesis and (5) PCR enrichment. After PCR, samples were cleaned up and size selection performed. Briefly, 2 μl of sample was subjected to Tapestation analysis to ascertain band sizes. Samples were run on 8% acrylamide gel at 100V for 1 hr, with correct size bands excised for gel purification. Small RNA libraries were diluted to 2 nM and run on a miSeq System (Illumina) for NGS using the V2 kit (Illumina). Data analysis was performed using the CLC Genomics Workbench (Qiagen) according the following data workflow: (1) Fastq files imported into the analysis suite, (2) sequences trimmed to remove poor quality and short reads, (3) trimmed reads run through the Small RNA Analysis pipeline, (4) extraction and counting, (5) annotation and count merging to identify expression level of each mapped miRNA. Mapped reads from individual samples were then compared to determine fold change for each miRNA.

miRNA Microarray and qPCR

Total RNA (500 ng) was isolated from exosomes using the miRCURY RNA Isolation Kit (Exiqon) and reverse-transcribed with the miScript II RT Kit (Qiagen). 20 ng of cDNA was used for qPCR with the human miFinder miRNA Array (Qiagen), with cycling conditions of 94° C. for 15 minutes, 40 cycles at 94° C. for 10 seconds, 55° C. for 30 seconds, 70° C. for 30 seconds, followed by melt curve analysis. The data were analyzed with the online miScript miRNA PCR Array data analysis tool. For validation of miRNAs identified by microarray and NGS, individual qPCR experiments were performed with miScript primer assays (Qiagen) using similar cycling and analysis schemes. Total RNA (2 μg) was also isolated from cells, as described above, for profiling of downstream miRNA targets by qPCR. The following custom primers were designed: CASP14 (F) 5′ gtt ccg aag aag acct gg at 3′ (SEQ ID NO: 31), (R) 5′ ttc tcc agc ttg acc atc tc 3′ (SEQ ID NO: 32); GALNT6 (F) 5′ gga gca cct aaa gga gaa gc 3′ (SEQ ID NO: 33), (R) 5′ ctg tct tgt cct cag cga tt 3′ (SEQ ID NO: 34); PAX5 (F) 5′ cat ccg gac aaa agt aca gc 3′ (SEQ ID NO: 35), (R) 5′ ace gga gac tec tga ata cc 3′ (SEQ ID NO: 36); PPM1F (F) 5′ ctt gge ttt cet gag aaa ca 3′ (SEQ ID NO: 37), (R) 5′ ctt gge ttt cet gag aaa ca 3′ (SEQ ID NO: 38); SUMO2 (F) 5′ atg gtt ctg tgg tge agt tt 3′ (SEQ ID NO: 39), (R) 5′ ctg ctg ttg gaa cac atc aa 3′ (SEQ ID NO: 40); j-Actin (F) 5′ atc etc ace ctg aag tac cc 3′ (SEQ ID NO: 41), (R) 5′ agc ctg gat agc aac gta ca 3′ (SEQ ID NO: 42), with cycling conditions of 95° C. for 15 minutes, 40 cycles at 94° C. for 15 seconds, 51° C. for 30 seconds, 72° C. for 30 seconds, followed by melt curve analysis. In some embodiments, the methods of preparing miRNA includes one or more of the above steps, including the use of the primers disclosed herein. Analyses were performed with Qiagen PCR Array Data Analysis Software, as described above.

Nucleofection of miRNA Mimics, miRNA Inhibitors and siRNA

Aged TNCs (10×106 cells per sample) were nucleofected with microRNA mimics (Qiagen), miRNA inhibitors (Qiagen), negative control siRNA (Qiagen), negative control miRNA inhibitor (Qiagen) or downstream target candidate siRNAs (Origene) using the Amaxa P3 Primary Cell 4D-Nucleofector X Kit (Lonza) on a 4D Nucleofector device (Lonza), according to manufacturer's specific protocol. Briefly, for nucleofection of CD34+ cells used in clonogenic assays, cells were nucleofected with 60 nmol total miRNA mimics, miRNA inhibitors or siRNA using the “human CD34+ cell” program. For nucleofection of T cells used in T cell activation and cell-mediated cytotoxicity assays, cells were nucleofected with 240 nmol total miRNA mimics, miRNA inhibitors or siRNA using the “human unstimulated T cell, high functionality” program.

miRNA Target Prediction and Network Analysis

Expression data from NGS was analyzed in silico by Ingenuity Pathway Analysis (IPA—Qiagen) to predict miRNA targets and downstream signaling networks. Differentially expressed exosomal and intracellular miRNA (1.4-fold cutoff) among young and aged isochronic, and aged isochronic and heterochronic samples, respectively, were uploaded to the IPA suite for Core Network Analysis. Predicted networks from the Core Analysis were then simultaneously likened using Comparison Analysis to identify the exosome-cell interactome during heterochronic restoration. Potential mRNA targets of candidate miRNAs were determined using the miRNA Target Filter. The source of the miRNA-mRNA relationship and the confidence of the relationship predictions were from TargetScan and the experimentally observed relationships were from TarBase. mRNA target selection was based on target rank score, where the highest ranked targets were common to the most candidate miRNA (score=6) and the lowest ranked targets to the least candidate miRNA (score=1). Potential interaction with the exosome-cell interactome was evaluated by creating a mock mRNA target expression profile (10-fold downregulation) to generate a Core Analysis network that could be likened using the Comparison Analysis tool. Candidates whose predicted networks converged with the interactome were selected for additional evaluation.

### Statistical Analysis

Statistical analyses were performed with ANOVA and Tukey-Kramer multiple comparisons test. For array and NGS expression analyses, average linkage was used for clustering and Pearson correlation analysis used for distance measurement to generate heatmaps and hierarchically cluster genes. p<0.05 was considered significant.

### Results

### Differences in Lymphohematopoietic Function Among Healthy Aged and Young Donors

The hematopoietic and immune systems are dependent on adult hematopoietic stem cell (HSC) function throughout an organism's lifetime to generate hematopoietic progenitor cells (HPCs) and mature effector blood cells. As these systems age or otherwise become dysfunctional, their diminishing functions lead to compensatory increases in immune-related diseases, including cancer. To this end, the overarching goal of this study was to identify novel factors produced by young healthy blood cells that could restore function to the aging or otherwise dysfunctional hematopoietic and immune systems.

To determine how blood cell function is solely impaired by temporal aging, without age-related co-morbidities, healthy study donors were recruited. Aged (>60 y/o) and young (18-29 y/o) donors were screened using a set of inclusion and exclusion criteria to select for the desired donor type (Table 4). General inclusion criteria such as normal BMI and no smoking, and exclusion criteria such no concurrent illness, abnormal BMI or co-morbidities yielded healthy aged donors. The criteria for young donors was more rigorous, as it was hypothesized that young individuals with extremely healthy lifestyles should yield the healthiest blood samples to identify anti-aging, restorative factors. Additional inclusion and exclusion criteria for the young cohort included exercise, diet and sleep requirements.

Compared to other stem cell-rich organs such as bone marrow (BM) and spleen, the systemic circulation contains minimal numbers of stem cells. To circumvent this, G-CSF-mobilized peripheral blood (MPB) was collected from aged and young study donors. MPB comprises a heterogeneous population of cells, including HSCs, HPCs, mesenchymal stem cells (MSCs), endothelial progenitor cells (EPCs), stroma and mature immune cells. In total, MPB from 5 young and 4 aged donors, with an average age of 22.2 and 60.8 y/o, respectively (Table 6) was obtained. Umbilical cord blood (UCB) was also collected for comparison to young MPB, since UCB comprises a similar hematopoietic matrix.

TABLE 6
Aged and Young Mobilized Blood Donor Demographics

| | | | | | | | TNC | CD34⁺ |
|---|---|---|---|---|---|---|---|---|
| | Donor | | | Height | Weight | | count | count |
| | ID | Age | Sex | (in) | (lb) | Ethnicity | (10⁹) | (10⁶) |
| 1 | A01 | 61 | M | 70 | 162 | Caucas. | 71.9 | 90.0 |
| 2 | A02 | 60 | M | 70 | 190 | Caucas. | 43.6 | 185 |
| 3 | A03 | 61 | M | 68 | 149 | Caucas. | 32.2 | 46.8 |
| 4 | A04 | 61 | M | 67 | 172 | Caucas. | 47.0 | 93.9 |
| 5 | Y01 | 28 | M | 67 | 140 | Afr. Am. | 42.2 | 354 |
| 6 | Y02 | 22 | M | 66 | 146 | Hispan. | 79.2 | 192 |
| 7 | Y03 | 20 | M | 67 | 160 | Caucas. | 45.5 | 200 |
| 8 | Y04 | 20 | F | 60 | 120 | Hispan. | 43.2 | 82.2 |
| 9 | Y05 | 21 | M | 69 | 130 | Hispan. | 20.1 | 47.1 |
| Aged | — | 60.8 | — | 68.8 | 168 | — | 48.7 | 103.9 |
| (n = 4) | | | | | | | | |
| Young | — | 22.2 | — | 65.8 | 139 | — | 46.0 | 175.1 |
| (n = 5) | | | | | | | | |

*Values for Aged and Young are averages of the 4 aged and 5 young donors enrolled in this study
**UCB was collected from 10 different donors. Information related to each donor was not made available

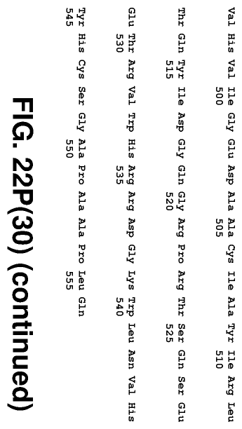

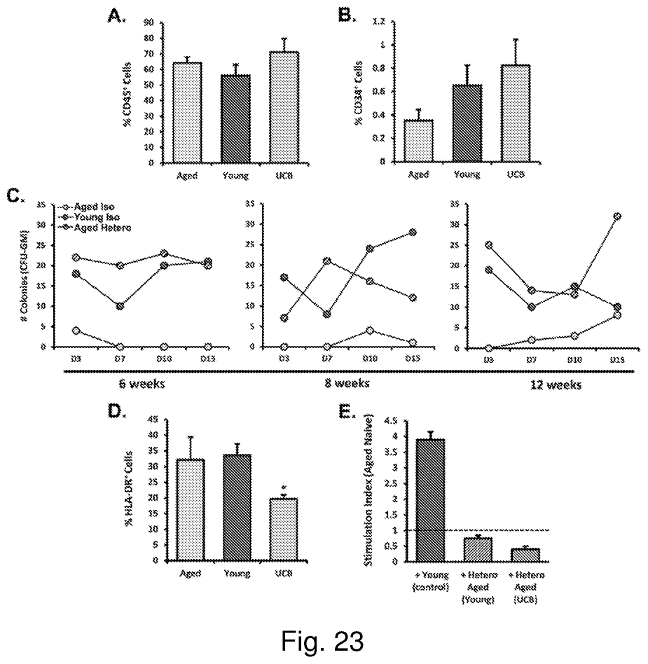

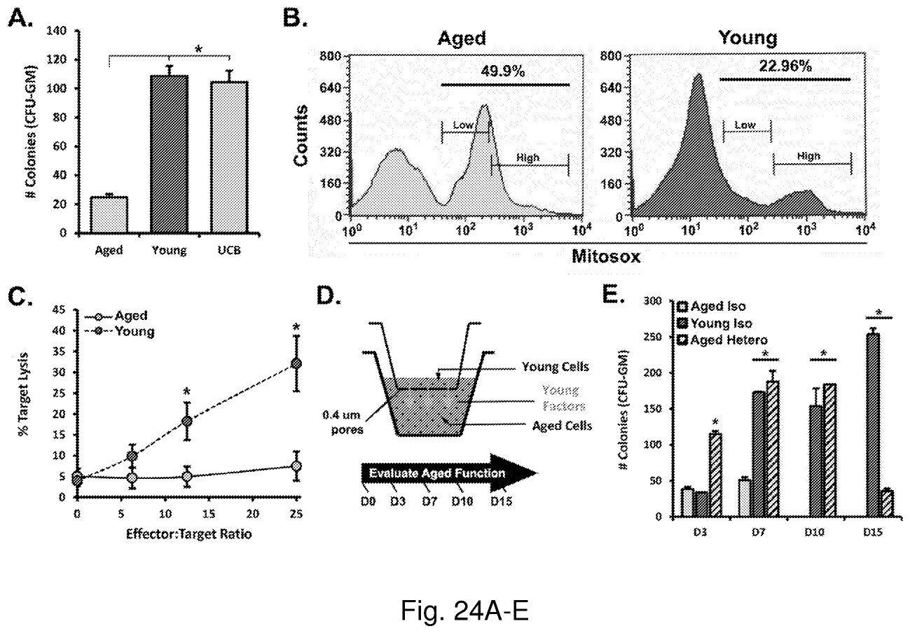

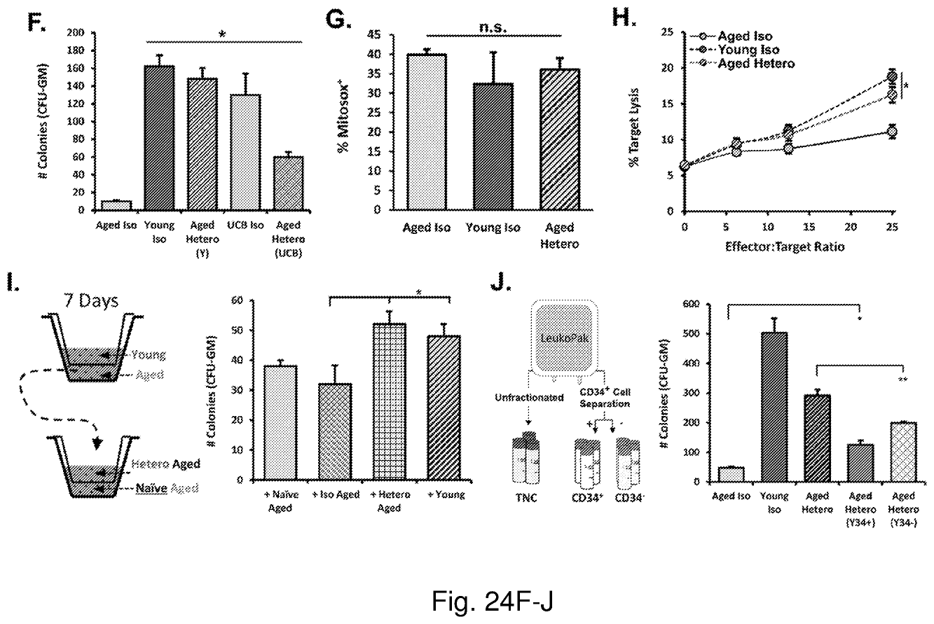

Collection of MPB from aged and young donors yielded a similar number of total nucleated (Table 6) and CD45+ cells (FIG. 23A), however young mobilization was more efficient as it yielded greater numbers of CD34+ cells (Table 6, FIG. 23B). Young CD34+ cells displayed significantly greater clonogenicity compared to aged, and were comparable to UCB (FIG. 24A). MPB cells from young donors also exhibited decreased oxidative stress (FIG. 24B), and increased cell-mediated cytotoxicity (FIG. 24C) compared to aged. These findings illustrate the striking impact of aging on the lymphohematopoietic system, even for donors with excellent health, on both the stem and mature effector cell compartments.

### Development of a Heterochronic Culture Model to Restore Aging Blood Cell Function

The functional decline of aging MPB was targeted through an adaptation of the heterochronic parabiosis model. The approach utilized heterochronic culture to harness production of paracrine factors from young MPB that could potentially restore aged function or function from dysfunction. Here, aged and young cells were separated by a transwell membrane that allowed contact-independent, cell-cell communication through exchange of soluble factors (FIG. 24D). The effect of heterochronic culture on aged MPB clonogenicity at periodic timepoints over 15 days in comparison to aged and young isochronic controls was tested. At day 7, a significant improvement in heterochronic vs. isochronic aged cell function was observed (FIG. 24E), and this effect was sustainable for up to 12 weeks as shown by LTC-IC assay (FIG. 24C). Induction of aged cell restoration was not restricted to young MPB cells, as a similar phenotype was observed in heterochronic cultures with UCB (FIG. 24F). Heterochronic culture altered aged MPB oxidative stress to a degree (FIG. 24G) and did significantly boost cell-mediated cytotoxicity (FIG. 24H). These effects were not due to potential transfer of immunogenic molecules secreted by young cells across the transwell membrane, as there were no differences in HLA-DR expression nor stimulation of naïve aged cells in one-way MLR (FIG. 23D and FIG. 23E).

To determine whether restoration can be propagated, aged restored (“hetero aged”) and non-restored (“iso aged”) cells were harvested after 7-day culture and seeded into fresh isochronic culture with naïve aged cells for an additional 7 days (FIG. 24I). Culture with cells from initial heterochronic culture induced a significant increase in clonogenicity compared to cells from initial isochronic culture, although these effects were not as robust as initial heterochronic culture (FIG. 24F).

Preliminary investigation into the young MPB population exerting these restorative effects revealed a role for CD34+ cells (FIG. 24J), as depletion significantly reduced heterochronic restoration. Similarly, culture of aged MPB with purified young CD34+ cells alone increased aged clonogenicity compared to isochronic control. The effect was not as robust as unfractionated heterochronic culture, thus implicating other young populations in the restorative mechanism.

### A Humanized Model of the Aging Lymphohematopoietic System to Study Restoration

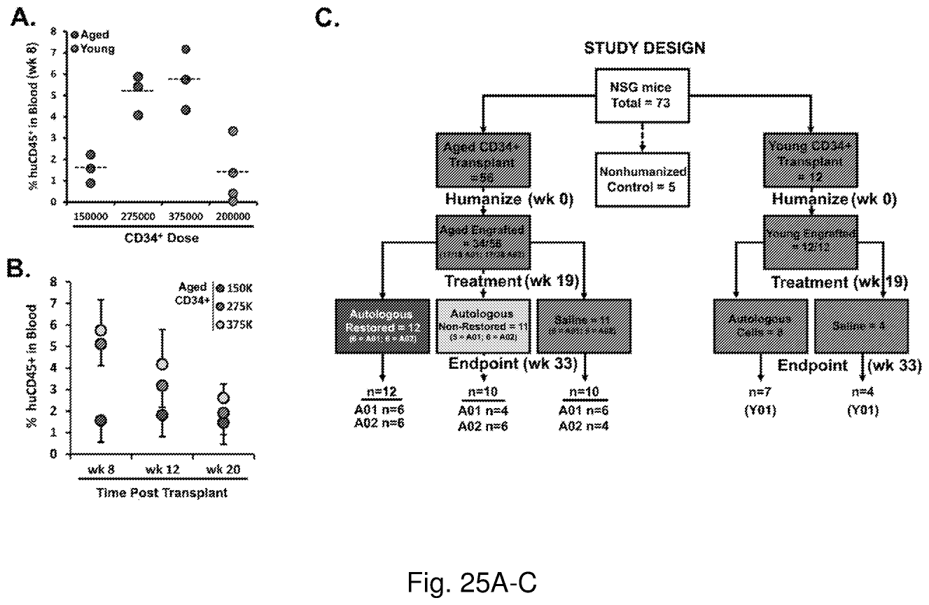

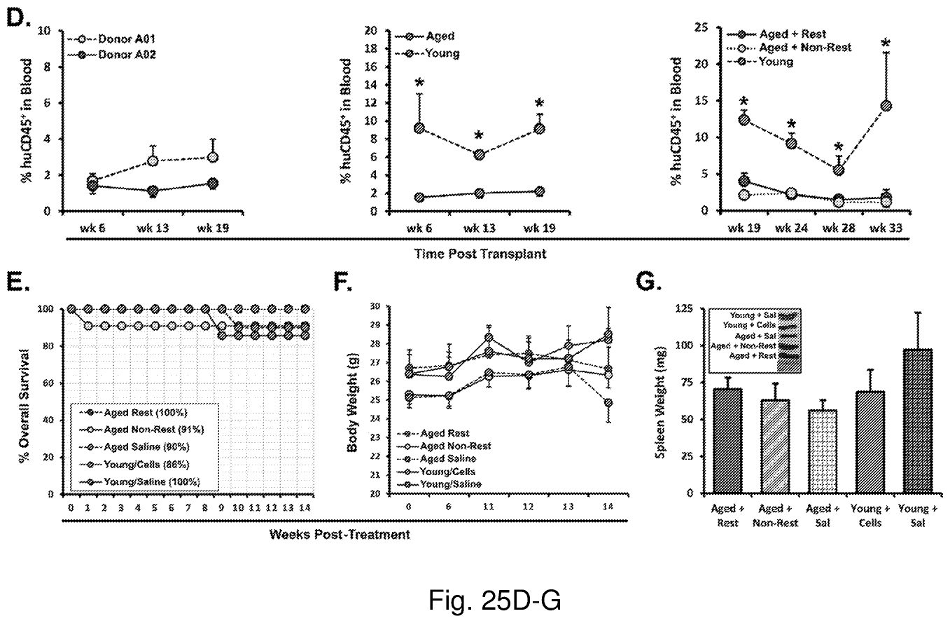

To translate the restorative findings from heterochronic MPB cultures, a sophisticated animal model was used that could recapitulate the aging human lymphohematopoietic system. NSG mice are a severely immunocompromised strain that lack an adaptive immune system and NK cells, thereby constituting a good model for human hematolymphoid engraftment. Most NSG humanization (huNSG) studies utilize young or primitive CD34+ cells isolated from sources such as MPB, UCB or fetal liver (FL). Implantation of these cell types would not be beneficial for the disclosed approach, since aged cell engraftment and hematopoiesis was required to study the therapeutic benefit of heterochronically-restored, autologous transplants. To this end, CD34+ cells isolated from aged study donor MPB collections were used to create aged huNSG mice. Pilot studies demonstrated a dose-escalating effect of aged CD34+ engraftment at 8 weeks post-transplant (FIG. 25A), which regressed and stabilized after 20 weeks (FIG. 25B).

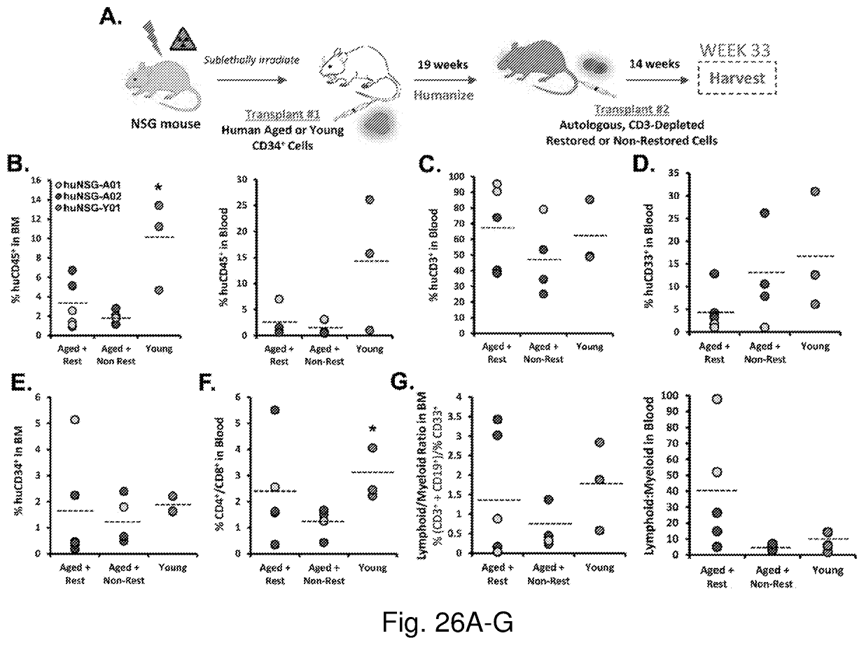

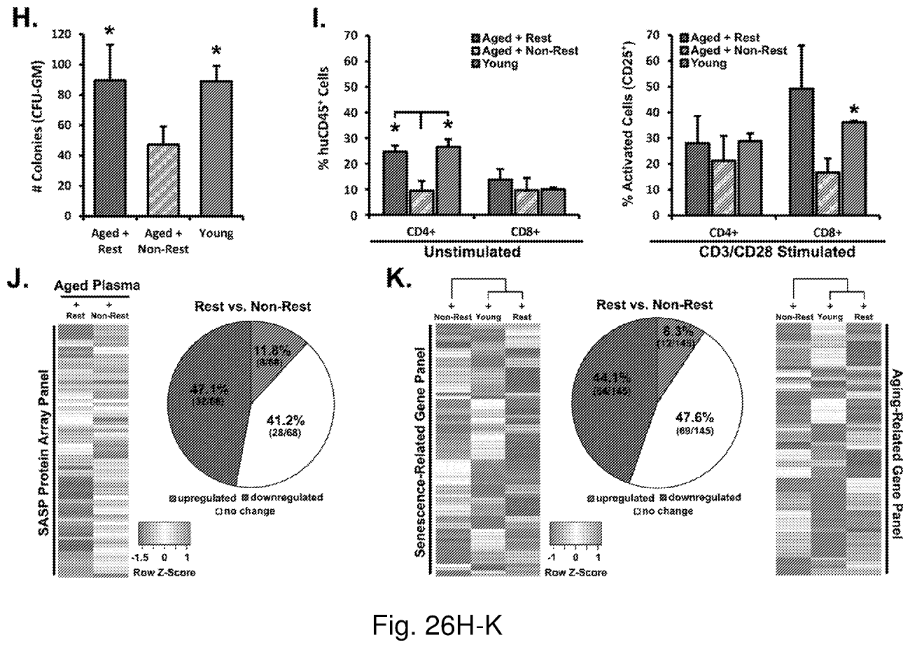

Next, this model was applied to study the effect of heterochronically-restored cells in autologous huNSG. Mice were transplanted with aged or young CD34+ cells and monitored for human engraftment and hematopoiesis over 19 weeks (FIG. 26A, FIG. 25C). Variability in huCD45+ chimerism was observed among individual aged donors (FIG. 25D, A01 vs. A02) as well among aged vs. young donor cell recipients (middle panel), with young transplants demonstrating significantly greater engraftment than aged. Mice exhibiting a minimum of 1% peripheral blood chimerism were enrolled in the treatment arm of the study and given a second autologous transplant of CD3-depleted MPB cells from heterochronic or isochronic culture. Mice exhibiting detectable chimerism less than 1% were injected with saline and utilized solely for safety profile comparison. After 14 weeks, mice were sacrificed and lymphohematopoietic phenotype and toxicological analyses performed.

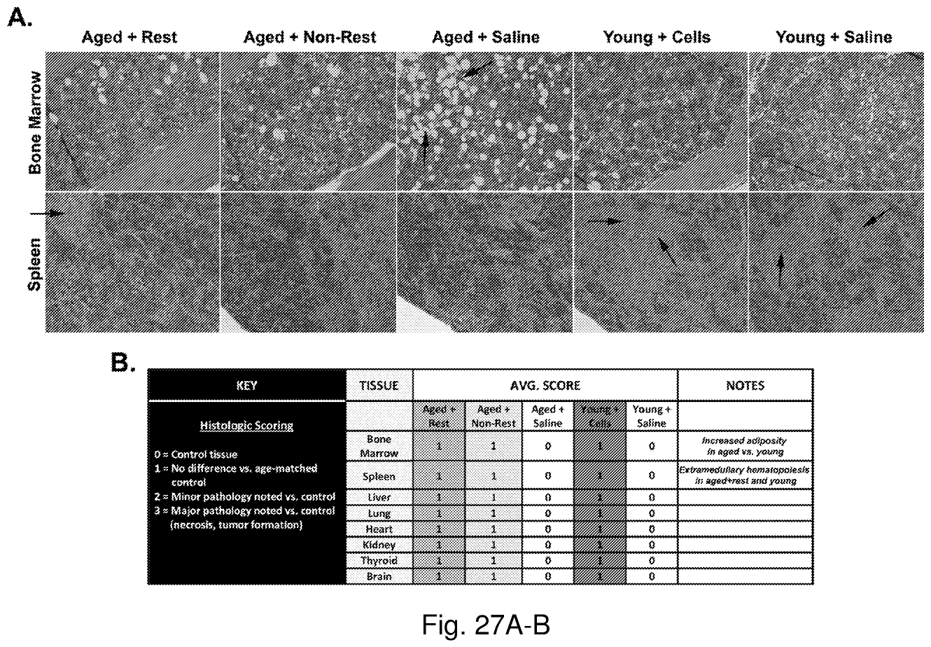

No significant safety concerns were observed for all treatment groups (FIG. 24E, FIG. 24F, FIG. 24G), with mice receiving heterochronically-restored (herein termed “restored huNSG”) and isochronically-non-restored (herein termed “non-restored huNSG”) cells exhibiting mean overall survivals of 100% and 91%, respectively (FIG. 24E). Histologic evaluation of immune tissues and major organs showed no evidence of tissue pathology or tumorigenesis (FIG. 27).

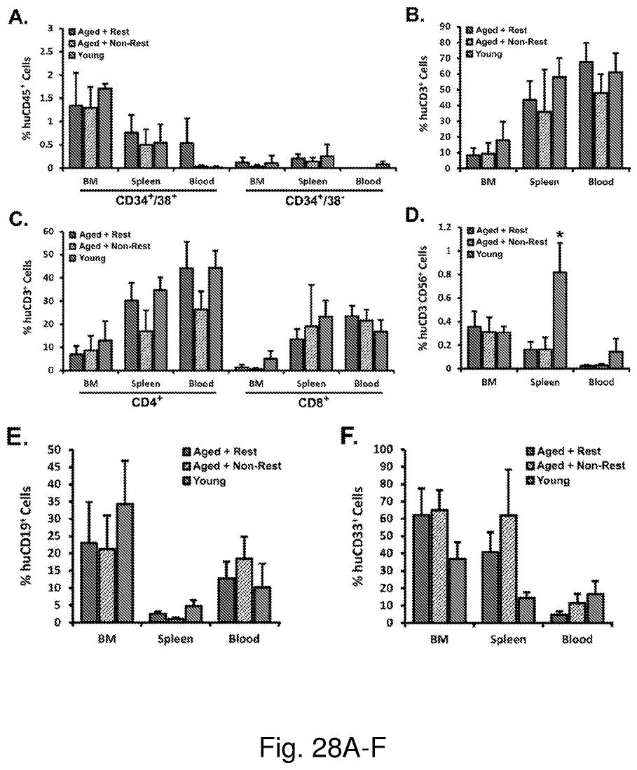

Phenotypic evaluation of human hematopoiesis was performed in blood, BM and spleen of treated huNSG (FIG. 26B-G, FIG. 28). Increased human chimerism was observed in BM of restored huNSG vs. non-restored (FIG. 26B, left plot). Restored transplants also demonstrated increased huCD3+(FIG. 26C) and decreased huCD33+(FIG. 26D) cell frequencies in blood compared to non-restored. No differences were observed in huCD34+ cell frequency in BM of all groups (FIG. 26E). To understand the phenotype in the context of relevant lymphohematopoietic aging-related metrics, the ratios of were determined huCD4+/CD8+ T cells (FIG. 26F) and lymphoid/myeloid cells (FIG. 26G) in blood and BM. Restored huNSG showed increased huCD4+/CD8+ T cell and lymphoid/myeloid ratios. These findings suggest that either the hematopoietic output, the autologous transplant itself or both in restored huNSG shows a decrease in age-related phenotype.

Next, human HSC/HPC and T cell function in BM and blood, respectively, of huNSG was evaluated. BM clonogenicity was significantly increased to levels comparable to young in restored huNSG (FIG. 26H). PBMCs harvested from restored mice and cultured ex vivo also showed increased frequency of huCD4+ cells (FIG. 26I, left plot). Stimulation of PBMCs with anti-CD3/CD28 elicited increased activation of huCD8+ cells from restored huNSG compared to non-restored (right plot).

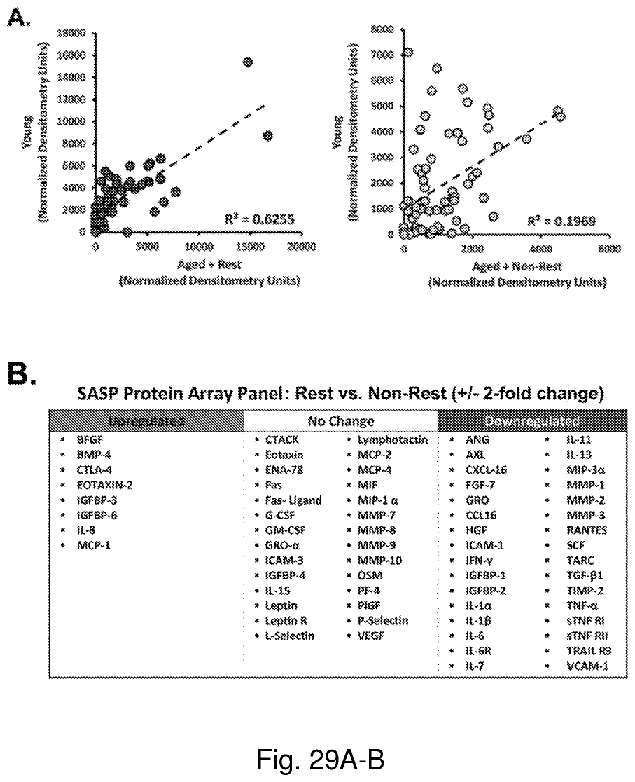

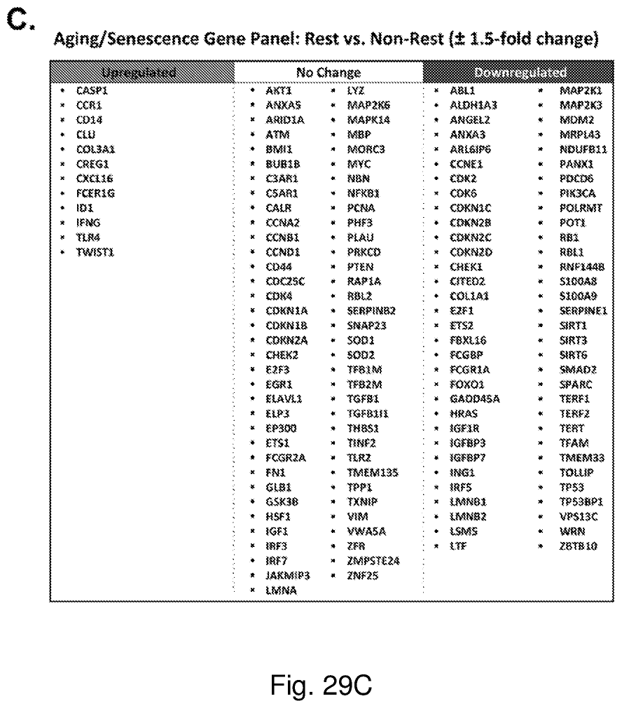

The final set of studies examined the effect of restoration on aging-related gene and protein expression in huNSG. Blood plasma isolated from restored and non-restored mice was probed for 68 factors linked to the senescence-associated secretory phenotype (SASP). Restored huNSG displayed decreased expression for 47% of factors and increased expression for only 12% of factors compared to non-restored (FIG. 26J, FIG. 29A-B). To determine whether these results were consistent with changes in gene expression for engrafted human cells, human cells were purified from chimeric BM and pathway-focused qPCR arrays were performed to evaluate 145 genes related to human senescence and aging. Similar to the expression pattern of the SASP study, purified cells from restored huNSG displayed decreased expression for 44% of factors and increased expression for only 8% of factors compared to non-restored (FIG. 26K, FIG. 29C). These findings illustrate that heterochronic restoration targets underlying pathways related to cellular aging and senescence. Further, the results suggest that the effect is propagated from the ectopically restored cells to the engrafted huBM compartment post-transplant.

### Young Exosomes Promote Heterochronic Restoration

The next set of studies sought to elucidate the mechanism of heterochronic restoration. It was surmised that due to the 0.4 μm pore size restriction of the transwell membrane, restorative young factors would likely be a complex acellular mixture of molecules capable of inducing global intracellular changes in aged target cells. Exosomes are small membrane vesicles released by all cell types, which contain a subset of proteins, lipids and nucleic acids derived from the parent cell. Exosomes have important roles in intercellular communication, both locally and systemically, as well as in regulating a number of aging-related signaling pathways in targeted cells. A role for exosomes in the restorative mechanism was investigated.

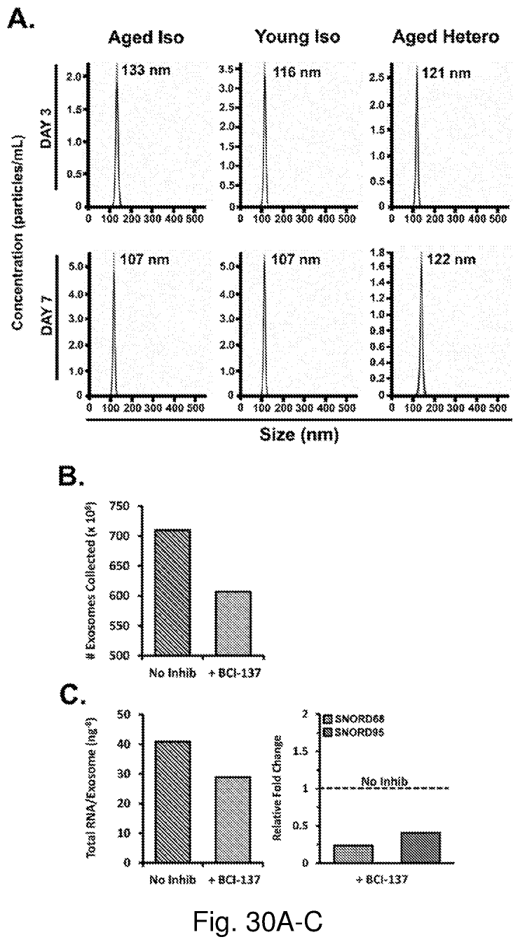

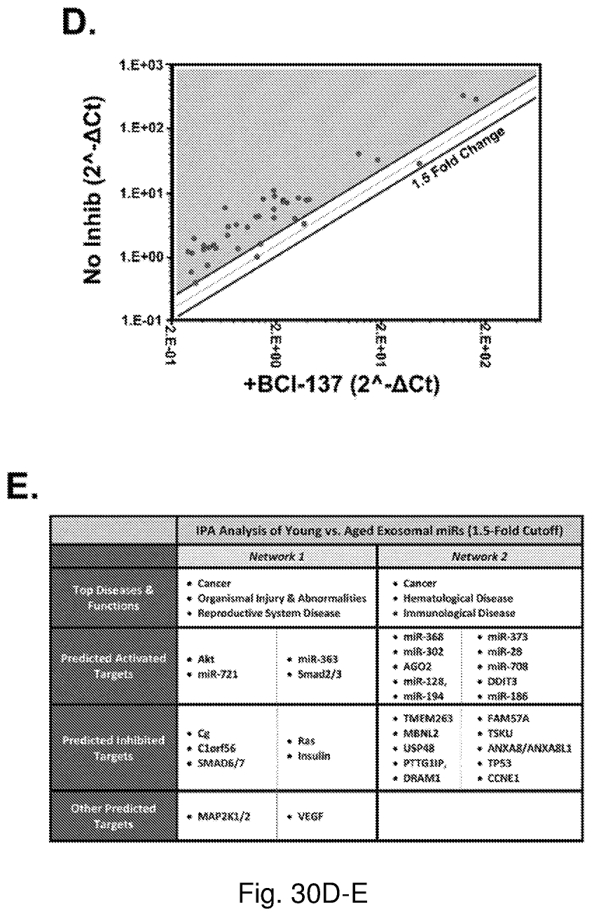

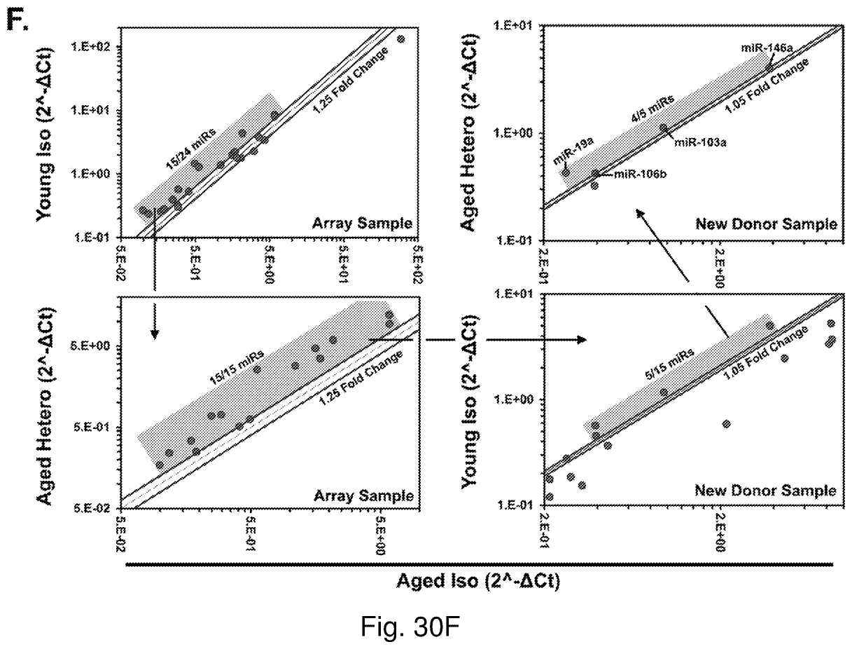

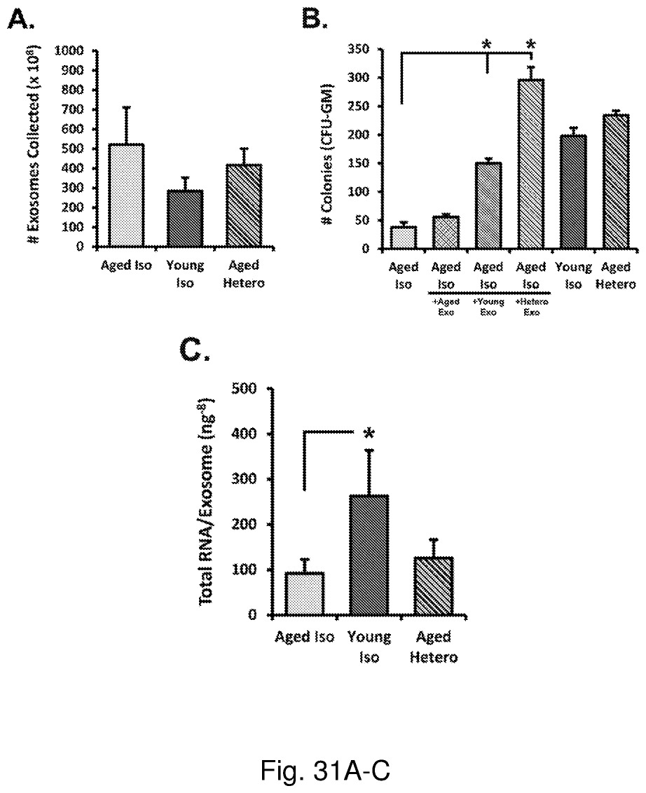

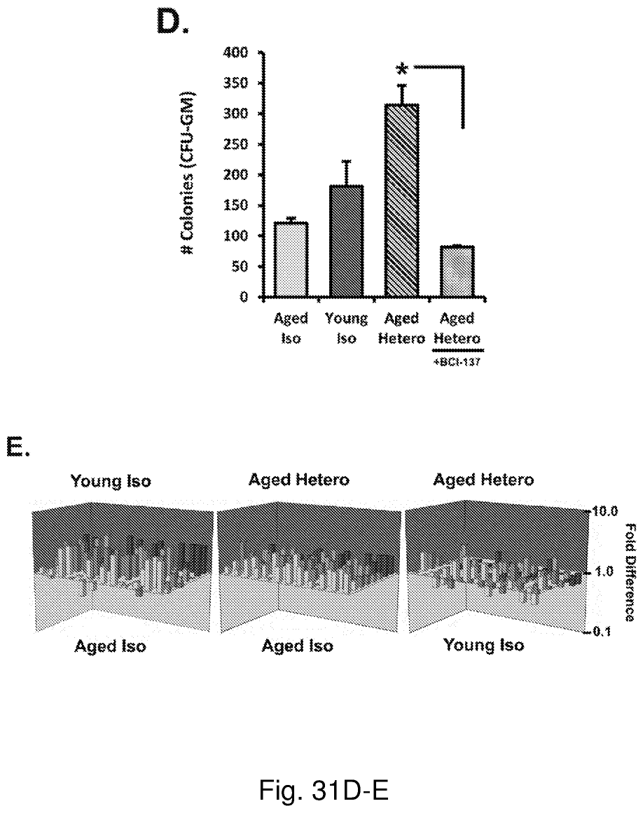

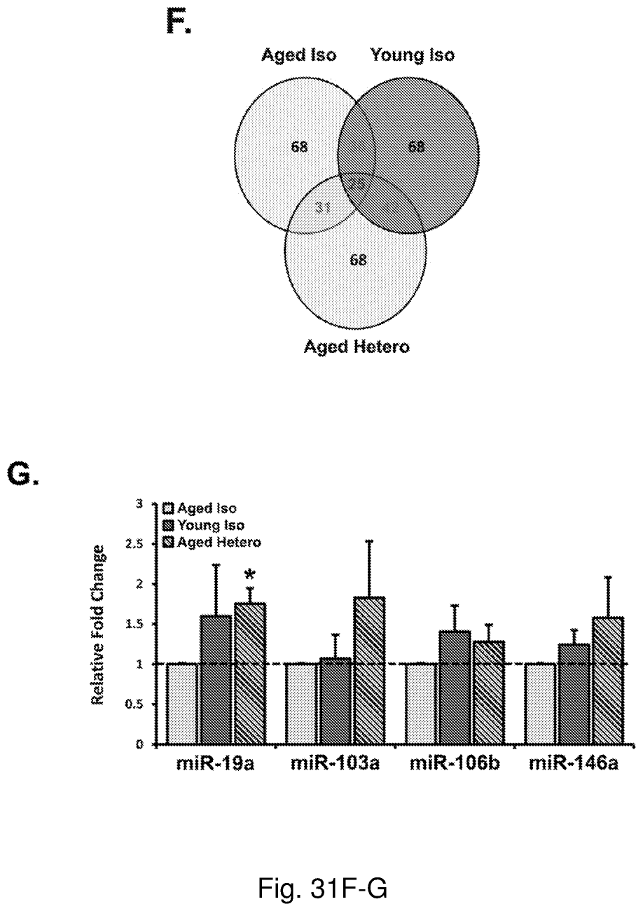

Examination of exosomes harvested from 7-day heterochronic and isochronic cultures showed minimal difference in particle size (FIG. 30A), and a modest increase in exosome production in aged vs. young isochronic cultures (FIG. 31A). Collected exosomes were then added to aged isochronic cultures to determine the effect on clonogenicity (FIG. 31B). Young and heterochronic, but not aged, exosomes produced a significant increase in aged isochronic clonogenicity. Blocking global exosome production with the nSmase inhibitor, GW4869, abrogated the restorative phenotype, but was also extremely toxic to the aged cultures (data not shown).

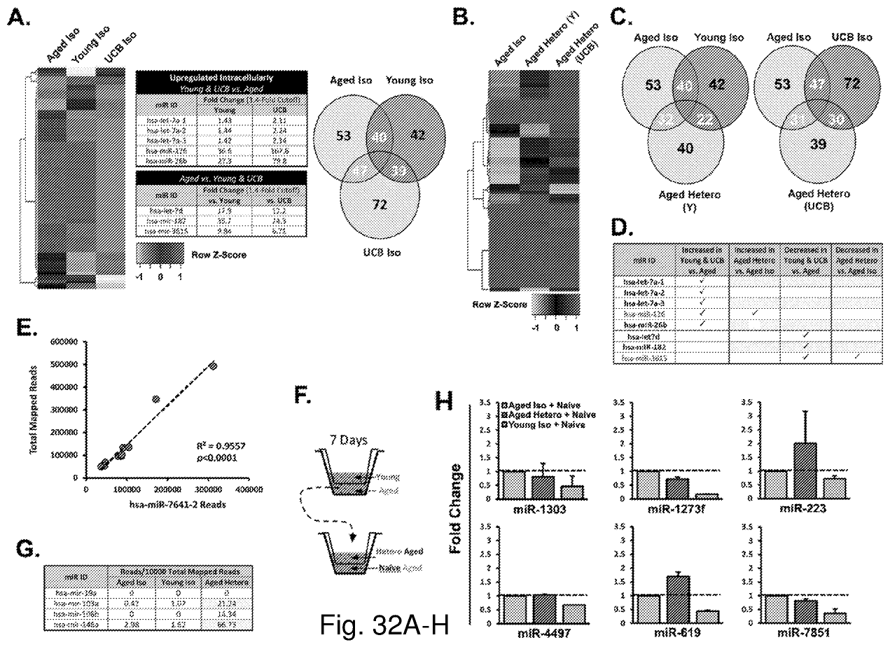

Though only a modest difference in exosome production was observed among aged and young cultures, the total RNA content of young exosomes was significantly greater than aged (FIG. 31C). Considering the importance of miRNAs in exosome-mediated intercellular communication, the role of miRNA in restoration was then investigated. The AGO2 inhibitor, BCI-137, was used to ablate miRNA packaging during exosome biogenesis. Treatment of heterochronic cultures with BCI-137 had minimal effect on exosome production and total RNA content (FIG. 30B and FIG. 30C), however it depleted the small and miRNA payload (FIG. 30D) and was coincident with a significant decrease in aged MPB clonogenicity (FIG. 31D). These findings led us to evaluate the miRNA profile of exosomes from isochronic and heterochronic cultures by utilizing an array with 84 probes for commonly expressed miRNA (FIG. 31E). A striking difference in exosomal miRNA expression was observed, with miRNA enrichment in young and heterochronic exosomes compared to aged, and only 25 of 68 detectable miRNAs expressed at similar levels for all cultures (FIG. 31F). Network analysis of the differential expression profile of young vs. aged demonstrated a number of predicted targets, including p53 (FIG. 30E). Differentially expressed miRNAs were validated in cultures from different donors, with miR-19a, miR-103a, miR-106b and miR-146a found to be consistently upregulated in young and heterochronic cultures compared to aged, although only minimally (FIG. 31G, FIG. 30E and FIG. 32G). Taken together, these data ascribed a putative role for young exosomes and their miRNA payload in the restorative mechanism.

Sequencing the Exosomal miRnome for Identification of Restorative miRNAs

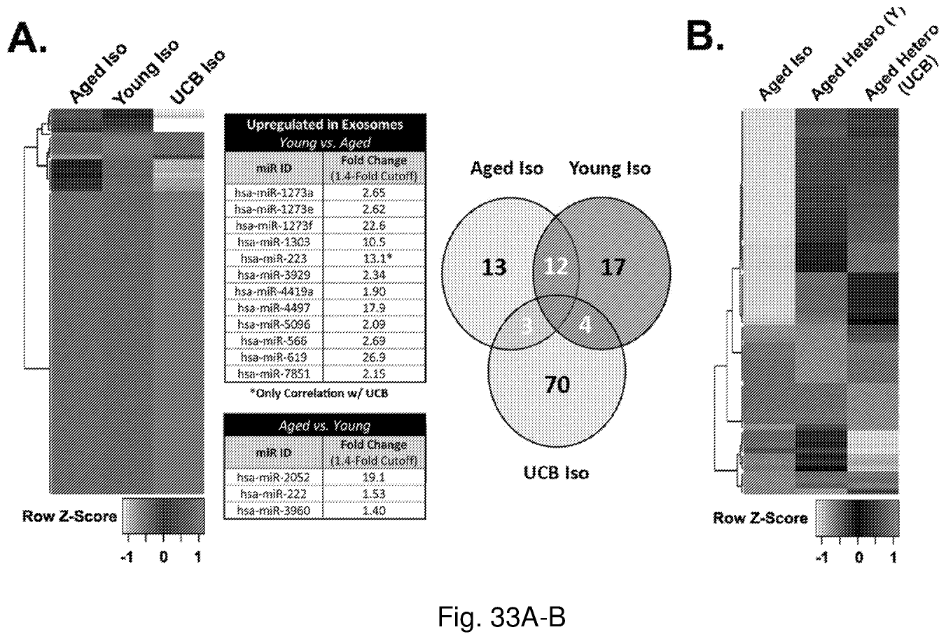

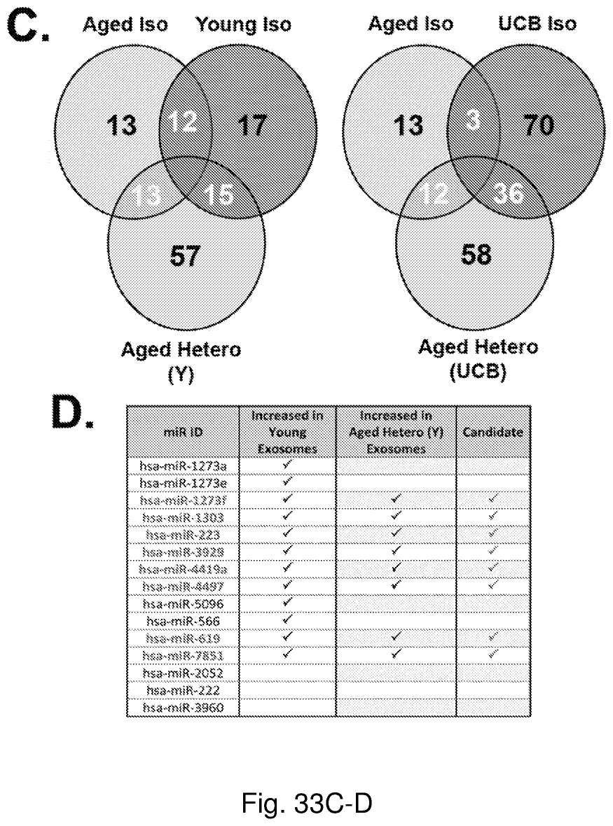

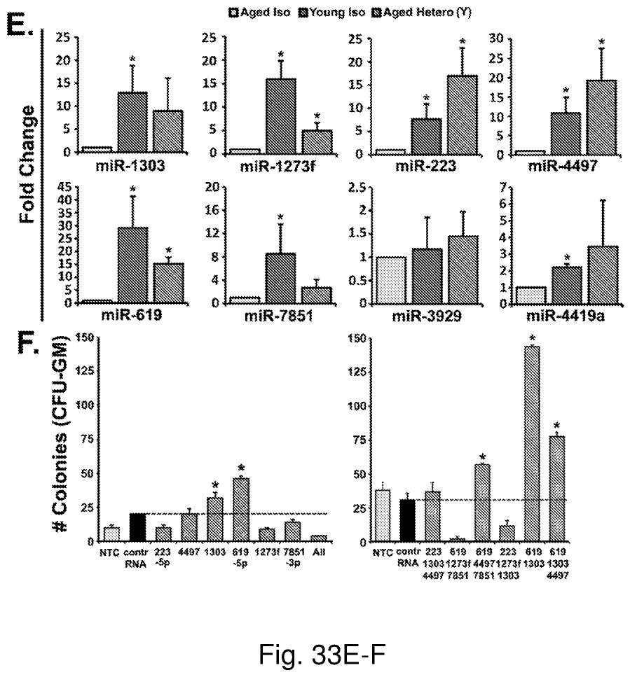

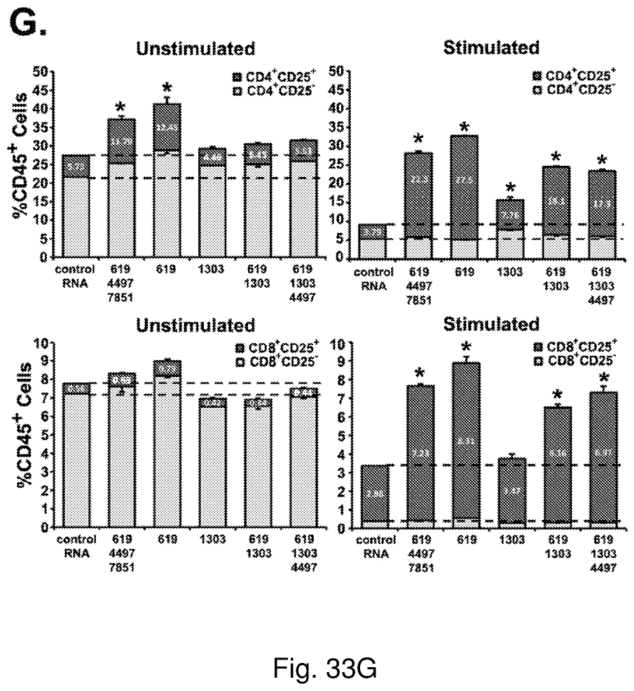

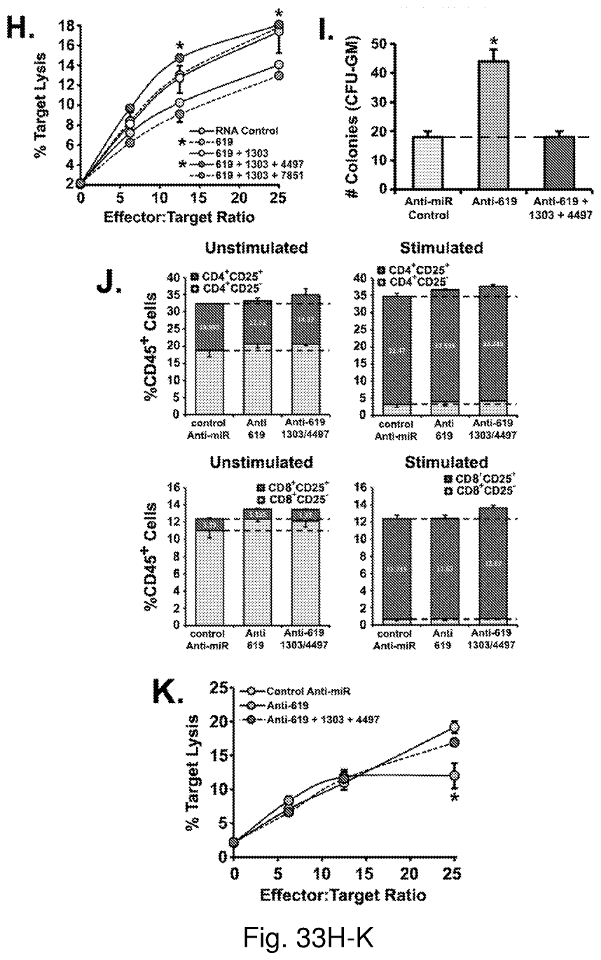

Initial exosome profiling of 84 commonly expressed miRNAs provided preliminary indication that young and aged exosomes exhibit distinctly different expression patterns. However, thousands of human miRNAs have been identified, thus a more comprehensive assessment of the exosomal miRnome was needed. To this end, deep sequencing was utilized and small RNA-Seq was performed to define the exosomal miRnome of aged, young, UCB and heterochronic cultures. An expression cutoff of 100 mappable reads in the sequencing dataset was defined, and 13 and 17 miRNAs were identified that met this criteria in aged and young exosomes, respectively (FIG. 33A). Interestingly, 12 of 17 exosomal miRNAs detected in young were expressed at significantly higher levels than aged, while only 3 of 17 were observed at lower levels. Examination of exosomes from UCB illustrated a vastly different miRnome, with 70 miRNAs of greater than 100 mapped reads and only 4 commonly expressed miRNAs as young observed. Similar findings were seen when comparing intracellular miRnomes among aged, young, and UCB cultures (FIG. 32A).

Next, the exosomal miRnome of aged cells was examined following heterochronic culture and a dramatic increase in miRNAs with expression above the 100-read threshold was found (FIG. 33B and FIG. 33C). The effect was ubiquitous to heterochronic culture, as similar exosomal and intracellular expression patterns were observed with aged culture of either young or UCB cells (FIG. 32B-D). Of the 12 differentially expressed young miRNAs (FIG. 33A), 8 were up-regulated following heterochronic culture (FIG. 33D) and 6 were validated for greater than 5-fold differential expression in young and heterochronic exosomes of additional study donors (FIG. 33E). Of note, due to a lack of widely accepted small RNAs for exosomal miRNA qPCR normalization, hsa-miR-7641-2 (FIG. 32E) was utilized since it was highly expressed in exosomes across all sequencing samples (aged, young, UCB, heterochronic) and significantly correlated with total mapped reads (p<0.0001). Of the 6 remaining candidates, only miR-223 and miR-619 were propagated in naïve aged cells after heterochronic restoration (FIG. 32F).

To screen the 6 miRNAs for restorative properties, first evaluated was the clonogenic effect of overexpressing candidate miRNAs in aged isochronic cultures (FIG. 33F). Individual expression of miR-619 or miR-1303 (left plot), or combinatorial expression of select miRNA formulations (right plot), elicited a significant effect on aged colony formation. Interestingly, of 5 formulations that caused a significant increase, 4 included miR-619 and 3 included miR-1303. These formulations were further evaluated to measure their effect on aged T cell activation (FIG. 33G). 2 formulations increased CD4+ T cell activation in unstimulated cells (top left plot), while 4 increased both CD4+ and CD8+ activation after stimulation (right plots). The 4 formulations underwent final screening by measuring their effect on cell-mediated cytotoxicity (FIG. 33H), with miR-619 alone or in combination with miR-1303 and miR-4497 showing significant increases in target lysis. Ascribing a definitive role for these miRNAs within young cells was not obvious from studies inhibiting miR-619 alone or in combination with miR-1303 and miR-4497 and evaluating the effect on clonogenicity (FIG. 33I), T cell activation (FIG. 33J) and cell-mediated cytotoxicity (FIG. 33K). No effect was observed in any of the assays after miR-combo inhibition, while inhibition of miR-619 alone produced a significant increase in young clonogenicity (FIG. 33I) and decrease in cell-mediated cytotoxicity (FIG. 33K).

Identification of Downstream Targets of Restorative miRNAs

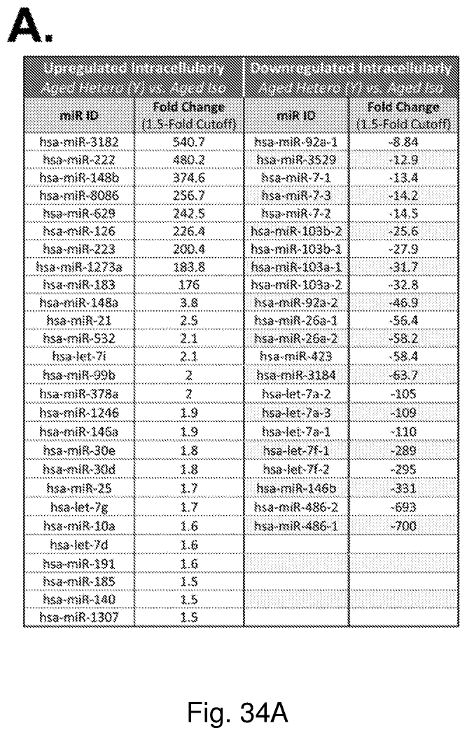

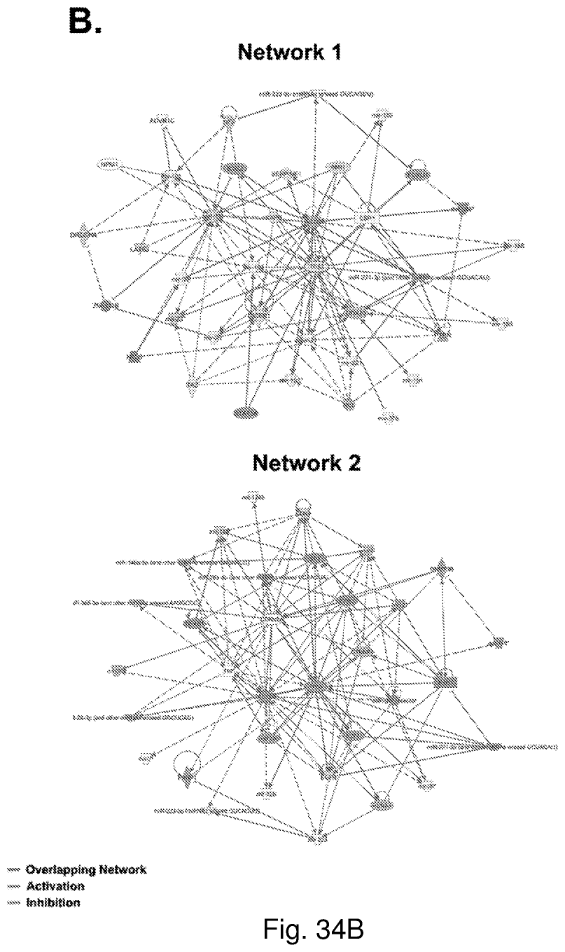

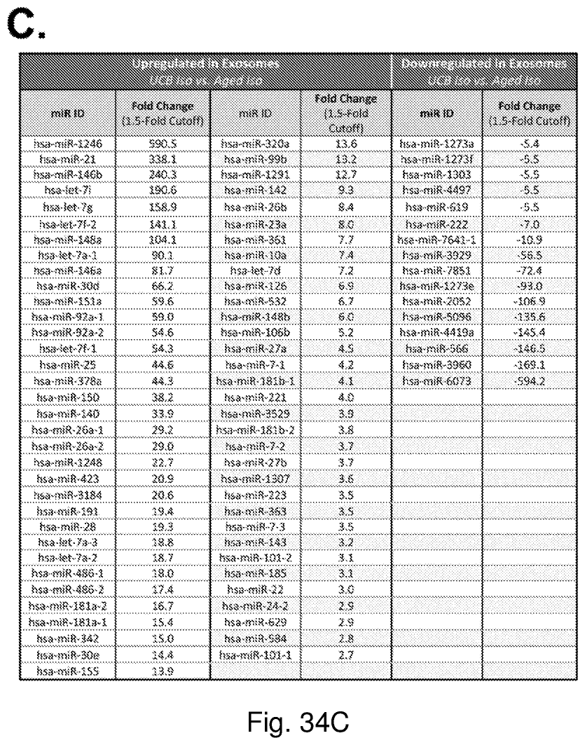

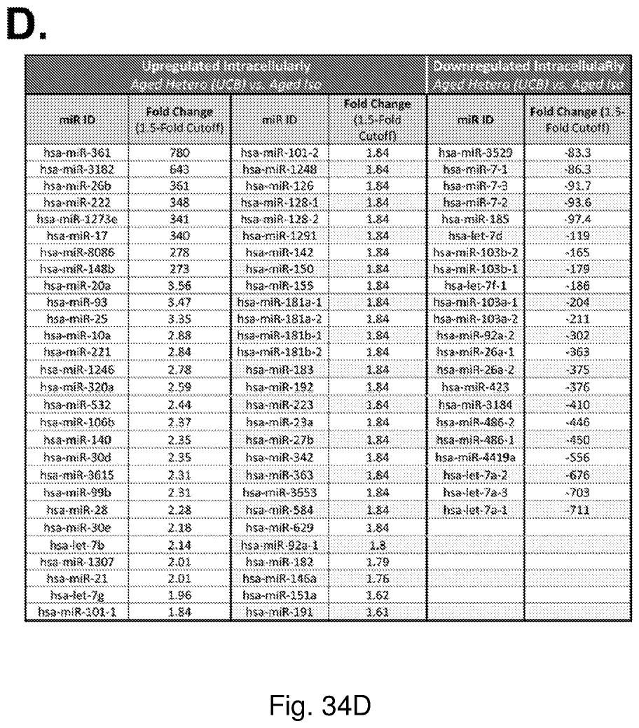

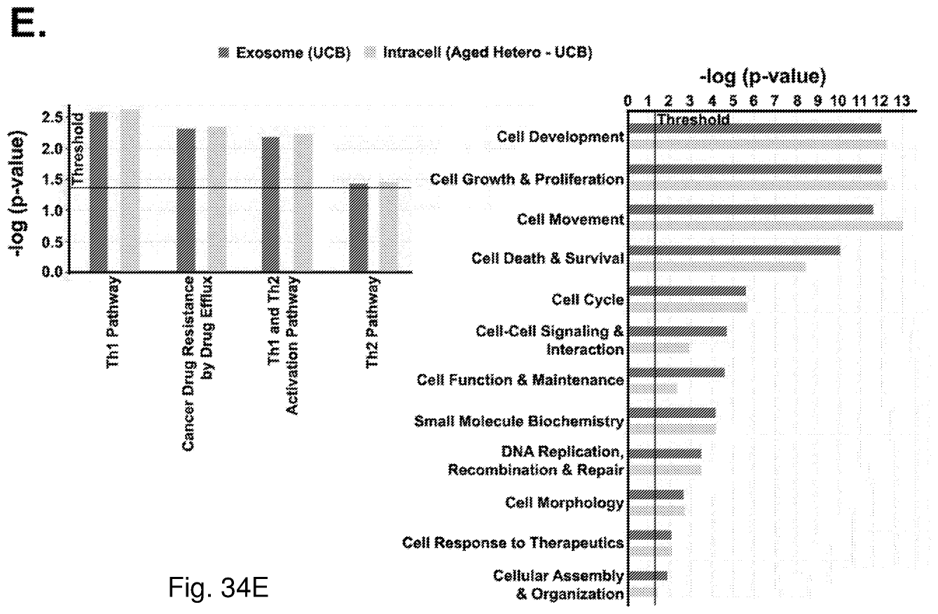

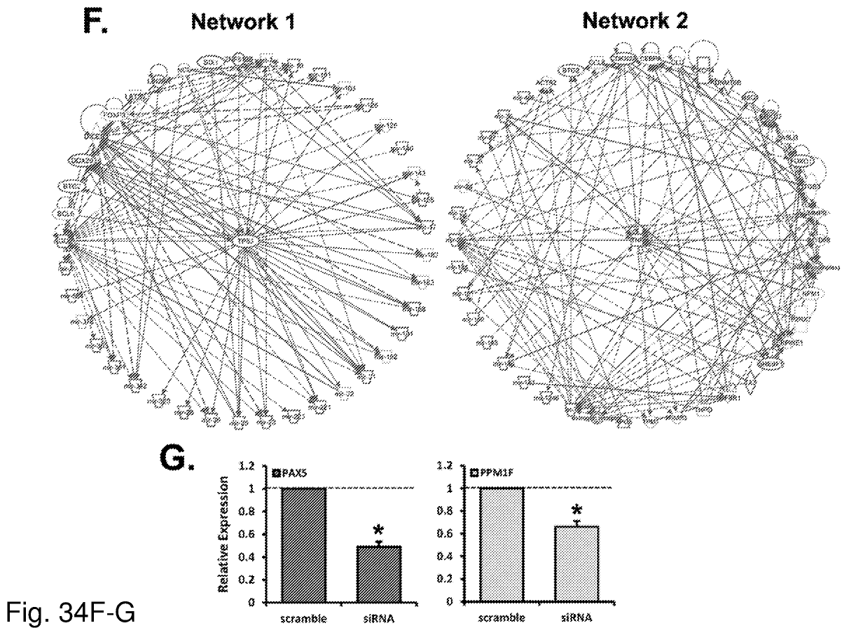

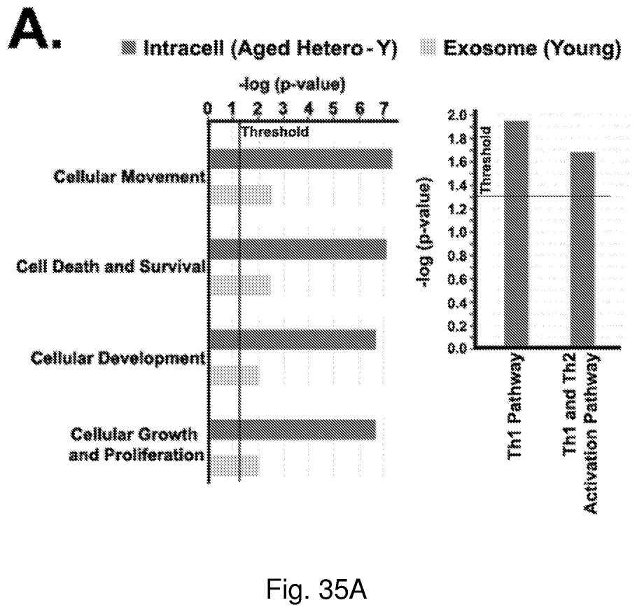

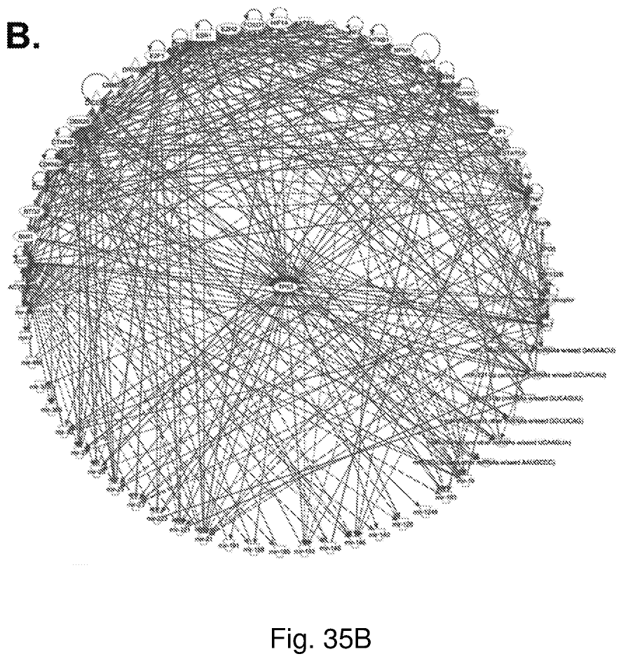

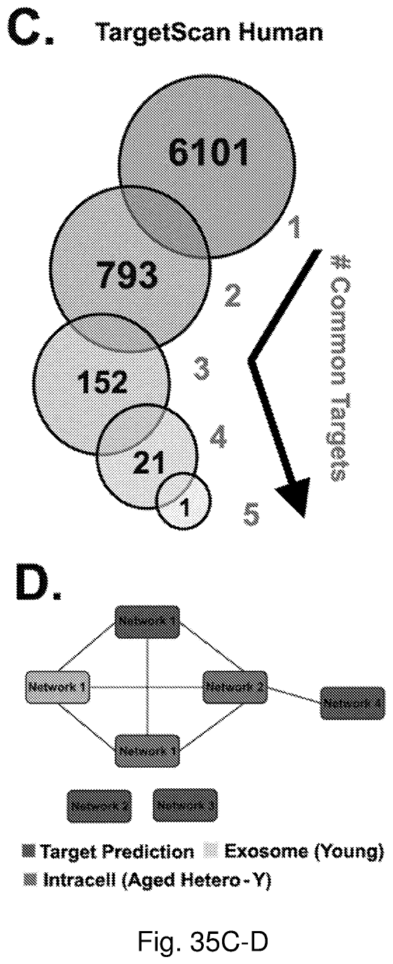

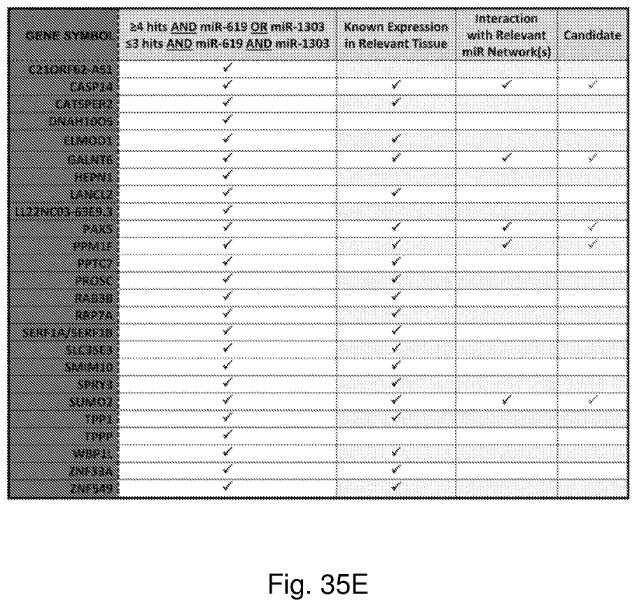

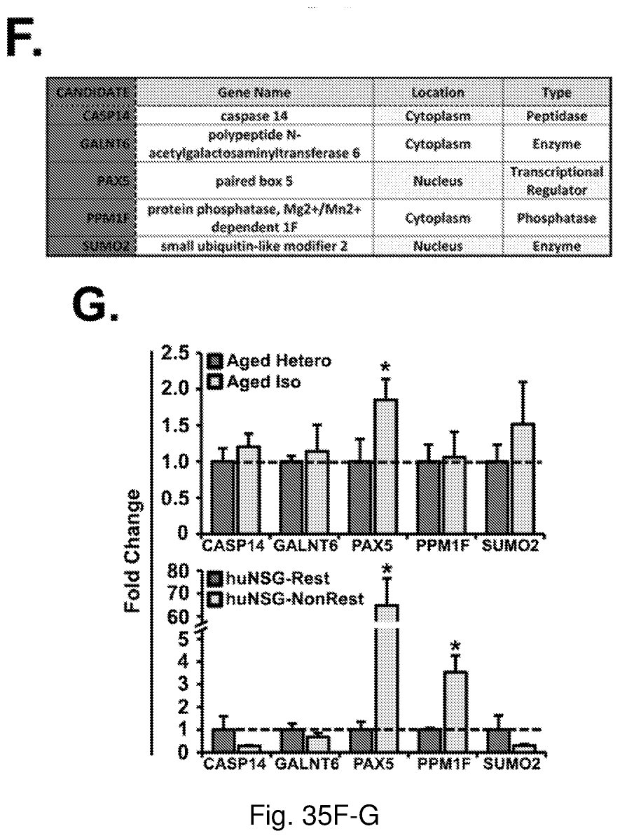

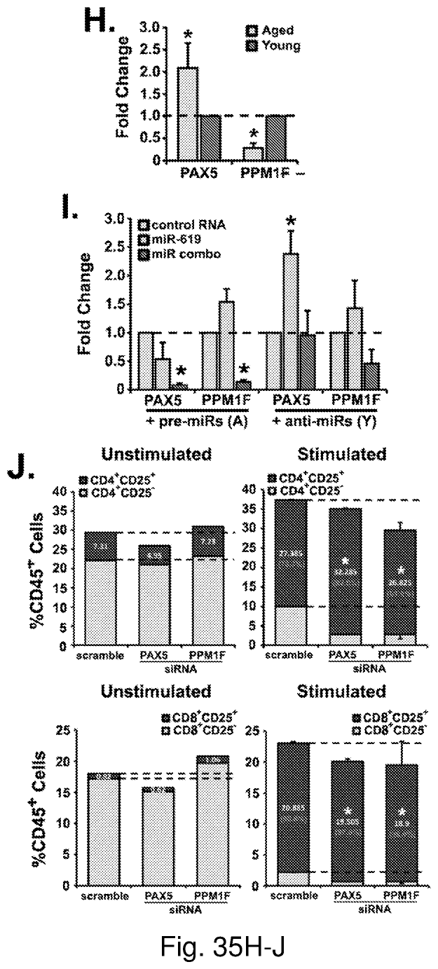

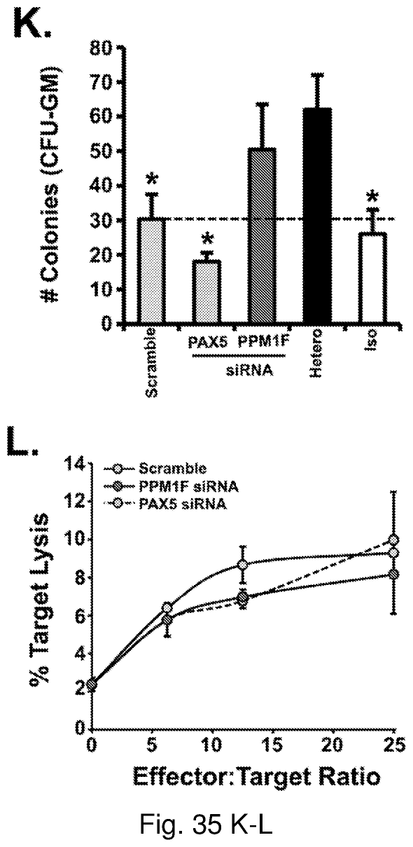

To fully describe the mechanism of heterochronic restoration, the downstream effector pathways targeted by the restorative miRNAs in aged cells was mapped. Network analysis utilizing the young exosomal (FIG. 33A) and aged heterochronic (young) intracellular (FIG. 27A and FIG. 34B) sequencing datasets was performed to map the miRNA interactome (FIG. 35A and FIG. 35B). A number of cellular functions (FIG. 35A, left plot) and canonical pathways (right plot), specifically in Th1 and Th2 cells, were predicted to be involved in this effector-target network. Many of the predicted molecules in the interactome are regulators of cellular senescence, including CDKN2A and p53, the latter of which displayed the greatest network convergence of the 2 datasets (FIG. 35B). Similar network analysis of the UCB exosomal (FIG. 34C) and aged heterochronic (UCB) intracellular (FIG. 34D) sequencing datasets identified related cellular functions, canonical pathways (FIG. 34E) and predicted molecules (FIG. 34F) as for young.

Next, miRNA target prediction software was used to identify direct targets of the 6 restorative miRNA candidates in aged cells. A total of 6101 targets were predicted for the individual miRNAs, so stratifying the number of common targets among the group was done (FIG. 35C). Targets sharing greater than 3 miRNA hits were further evaluated based on the presence or absence of miR-619 and miR-1303, which were shown as significant to restoration (FIG. 33). The remaining targets were first scanned for known expression in relevant tissues, as targets encoding hypothetical proteins and whose expression was restricted to neural tissues were eliminated, and then predicted pathway analysis (FIG. 35D) in the context of the effector-target interactome (FIG. 35D; FIG. 35E) to reveal 5 target candidates (FIG. 35F). Of these 5 candidates, PAX5 was significantly downregulated in aged cells from heterochronic culture (FIG. 35G, top panel), while both PAX5 and PPM1F were downregulated in human cells purified from BM of huNSG restored mice compared to non-restored (bottom panel). Basal expression of PAX5 and PPM1F in aged vs. young cells was drastically different, as PAX5 was significantly elevated in aged vs. young cells (FIG. 35H, left bars), while the opposite was true for PPM1F (right bars).

To demonstrate a cause-effect relationship between the miRNA candidates and the predicted downstream targets, aged cells were treated with miR-619 alone or in combination with miR-1303 and miR-4497 (miR-combo), or control RNA, and measured the effect on target gene expression (FIG. 35I, left bar sets). Both targets were significantly decreased following treatment with the miR-combo formulation. A similar effect on target expression was not observed after inhibiting miR-619, alone or in combination, in young cells (right bar sets).

The next set of studies knocked down PAX5 or PPM1F in aged cells (FIG. 34G) and measured effects on T cell activation, clonogenicity and cell-mediated cytotoxicity (FIG. 35J-L). Knockdown of either candidate did not significantly alter T cell activation of unstimulated cells (FIG. 35J, left panels), however both knockdowns displayed a significantly greater percentage of activated CD4+ (top right panel) and CD8+ (bottom right panel) T cells following stimulation. siRNA knockdown of PPM1F but not PAX5 yielded an increase in aged cell clonogenicity (FIG. 35K) compared to both scrambled RNA and isochronic controls. No significant difference between PPM1F knockdown and heterochronic control was observed, while all other variables were significantly decreased. No effect on cell-mediated cytotoxicity was observed for either knockdown in aged cells (FIG. 35L).

Aged Cell Restoration with miRNA Candidates in the Humanized Mouse Model

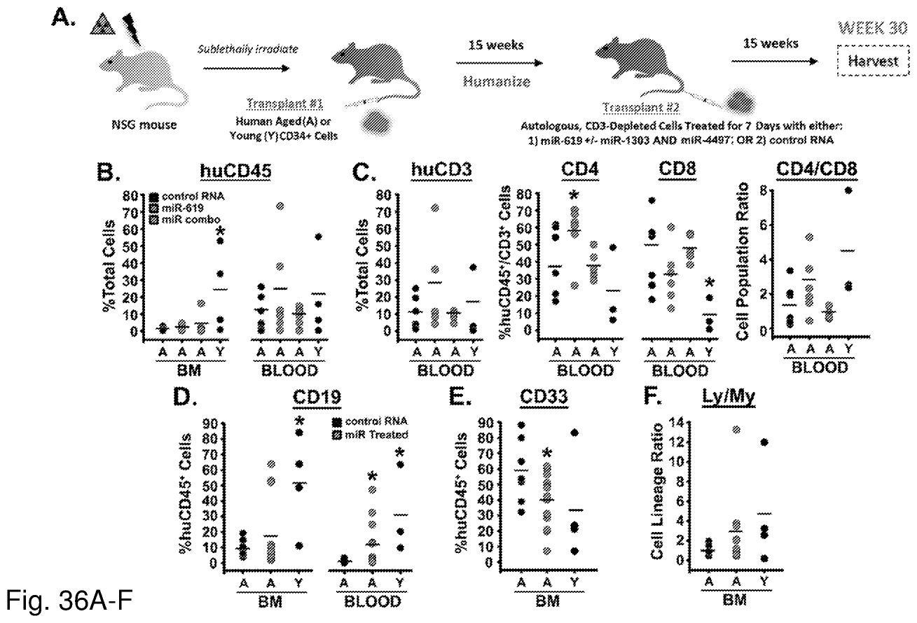

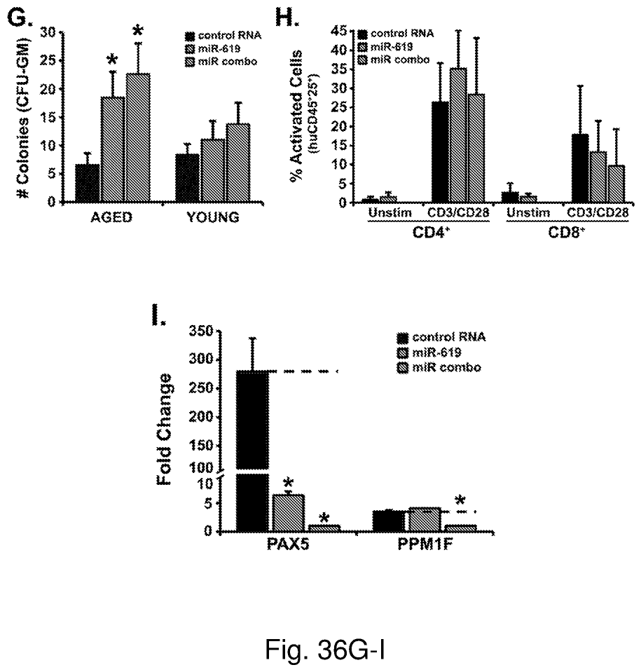

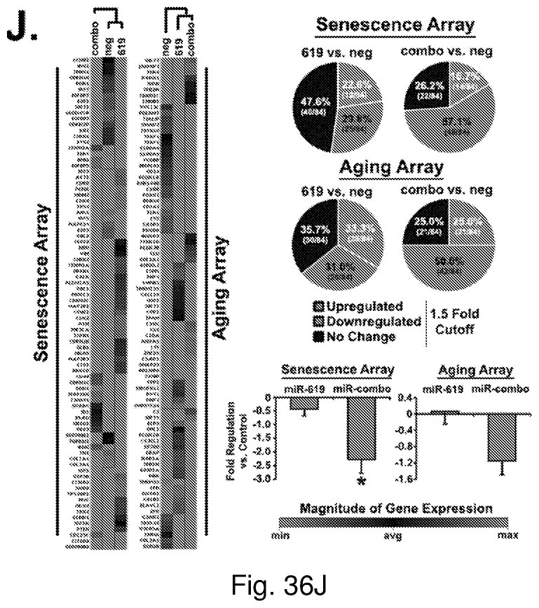

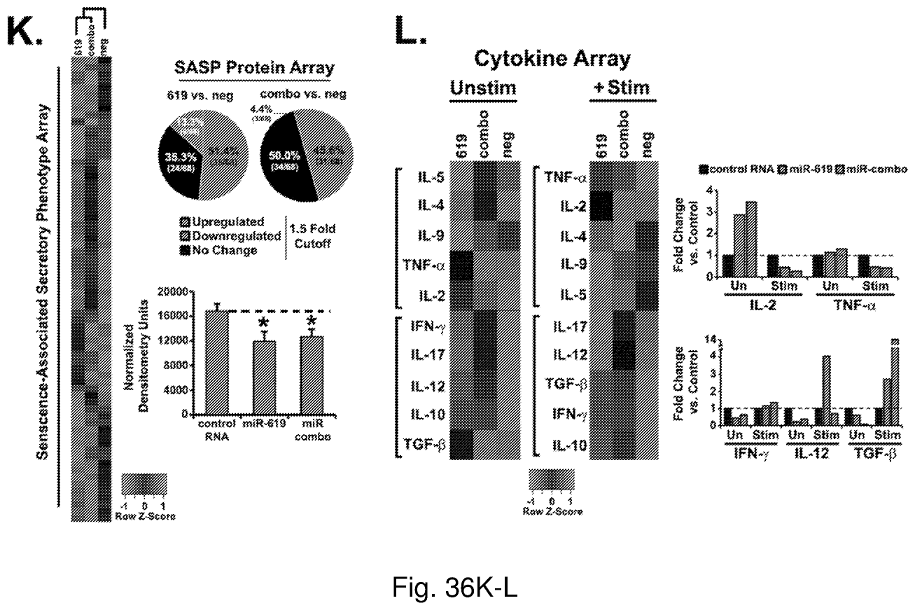

The study then examined whether the restorative phenotype induced by the candidate miRNAs (FIG. 36) could produce a similar effect as heterochronic restoration (FIG. 25) in mice engrafted with an aged human immune system. Mice were again transplanted with aged or young CD34+ cells and monitored for human engraftment and hematopoiesis over 15 weeks (FIG. 36A, FIG. 28A). Variability in huCD45+ chimerism was observed among individual young (FIG. 28B, Y03 vs. Y04) and aged (A03 vs. A04) donors. Mice exhibiting a minimum of 1% peripheral blood chimerism were enrolled in the treatment arm of the study and given a second autologous transplant of CD3-depleted MPB cells from 7-day cultures of cells transfected with miR-619 alone or in combination (miR-combo) with miR-1303 and miR-4497, or control RNA. After 15 weeks, mice were sacrificed and lymphohematopoietic phenotype and toxicological analyses performed.

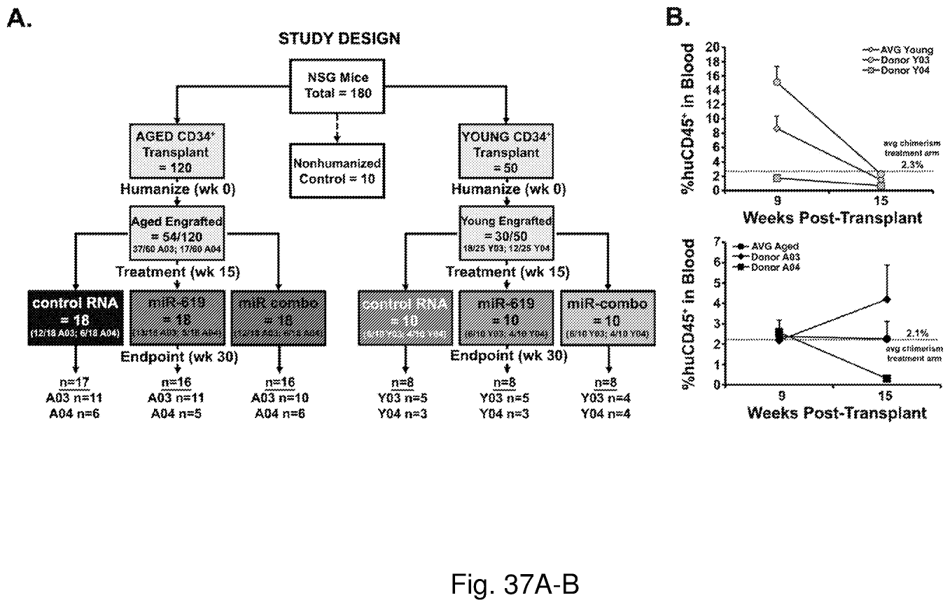

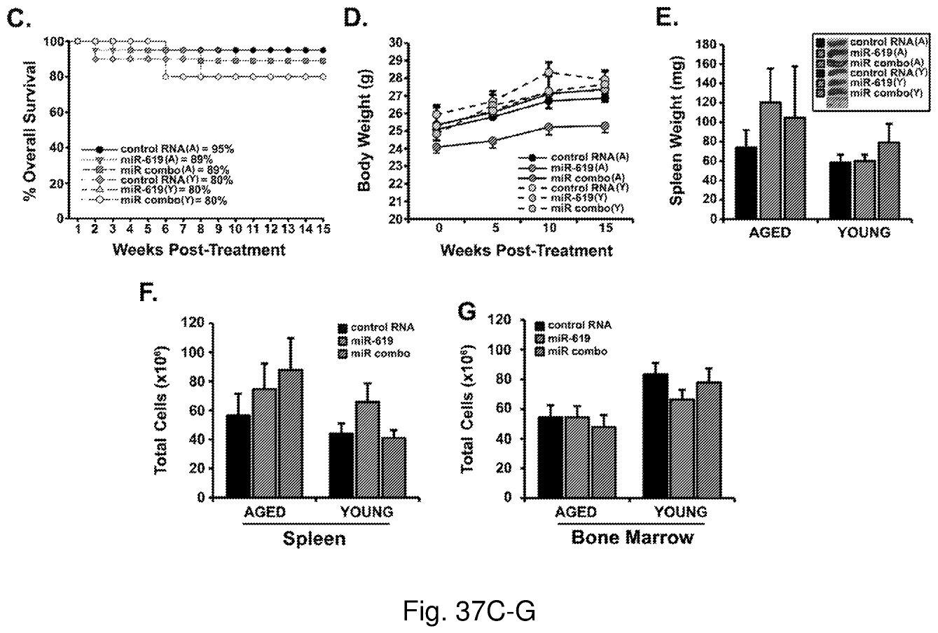

No significant safety concerns were observed for all treatment groups (FIG. 37C-E), with mice receiving aged cells treated with miR-619 alone or in combination exhibiting mean overall survivals of 89% and 89%, respectively, compared to 95% for control (FIG. 37C). Total cellularity in BM and spleen from mice transplanted with aged and young cells were unremarkable for all groups (FIG. 37F). Histologic evaluation of immune tissues and major organs showed no evidence of tissue pathology or tumorigenesis (FIG. 38). Enlarged spleens were observed among several mice within both study treatment and control arms. These mice showed increased splenic cellularity due to extramedullary hematopoiesis.

Phenotypic evaluation of human hematopoiesis was performed in blood and BM of miRNA-treated huNSG (FIG. 36B-F). No significant difference in human chimerism was observed in BM or blood of aged miR-619- (red circles) or miR-combo-treated transplants compared to control (FIG. 36B). Mice transplanted with aged miR-619 cells demonstrated increased huCD3+(FIG. 36C, far left panel) and significantly increased huCD4+(middle, left panel) cells in blood compared to RNA control. These findings were concomitant with decreased huCD8+ cells (middle, right panel) and increased huCD4+/CD8+ ratio (far right panel) in blood of aged miR-619 transplants vs. control. When the mice receiving aged cells treated with either miRNA formulation were grouped into a single cohort, a statistically significant increase was observed in huCD19+ output in blood compared to control (FIG. 36D). Coincident with the increase in lymphoid output, a significant decrease in aged huCD33+ myeloid production in the BM of miR-treated mice compared to control (FIG. 36E) was observed. Together, these data yielded an increased aged lymphoid to myeloid ratio in BM of miR-treated mice compared to control (FIG. 36F). These findings suggest that in aged huNSG transplanted with miR-treated cells, the hematopoietic output demonstrates a decrease in age-related phenotype.

Next, human HSC/HPC and T cell function was evaluated in BM and blood, respectively, of huNSG transplanted with miR-treated aged cells. BM clonogenicity was significantly increased in mice receiving aged cells treated with either miR-619 or miR-combo compared to control (FIG. 36G). PBMCs harvested from all treatment groups were cultured ex vivo but did not show any significant difference in huCD4+ or huCD8+ activation following stimulation with anti-CD3/CD28 compared to control (FIG. 36H).

The final set of studies examined the effect of miRNA-based restoration on aging-related gene and protein expression in huNSG, as well as the effect on the predicted targets PAX5 and PPM1F (FIG. 35). Human cells were purified from chimeric BM to first evaluate target gene expression (FIG. 36I), and second to perform pathway-focused qPCR arrays to evaluate 145 genes related to human senescence and aging (FIG. 36J, FIG. 39). Human cells isolated from the BM of huNSG transplanted with aged cells treated with the miR-combo formulation showed a significant decrease in PAX5 (FIG. 36I, left set of bars) and PPM1F (right set of bars) expression.

For the gene expression arrays, cells isolated from mice treated with the miR-combo formulation also displayed a decrease in more than half of all genes linked to senescence and aging (FIG. 36J, heat maps and pie charts). This correlated with a significant fold-decrease in the senescence array (left bar graph) and a substantial fold-decrease in the aging array (right bar graph). Next, blood plasma isolated from miR-treated and control mice was probed for 68 factors linked to the SASP (FIG. 36K). Similar to the expression pattern of the gene array studies, plasma from miR-619 and miR-combo treated mice showed decreases in 51.4% and 45.6% of SASFs, respectively (heat map and pie charts), with significant decreases in total expression (bar graph) compared to control.

It was then examined whether PBMCs harvested from the blood of all treatment groups, cultured ex vivo and stimulated with anti-CD3/CD28 differentially produced T cell cytokines (FIG. 36L). Conditioned media collected from the cultures after 72 h were used to probe human T cell cytokine arrays. Results showed a predominantly Th2 cytokine profile (IL-4, IL-5, IL-9) in miR-treated cells compared to control prior to stimulation (left heat map), with a switch to Th1 cytokine profile (IFNγ, IL-12, IL-10) following stimulation (right heat map). Increased production of both TGFβ and IL-17 following stimulation also indicated the presence of regulatory T cells and Th17 cells, respectively. Taken together, these data suggest a functional response to a T cell stimulus by phenotypic switching and immune activation.

Comparison of Heterochronic Vs. miRNA-Based Restoration in the huNSG Model

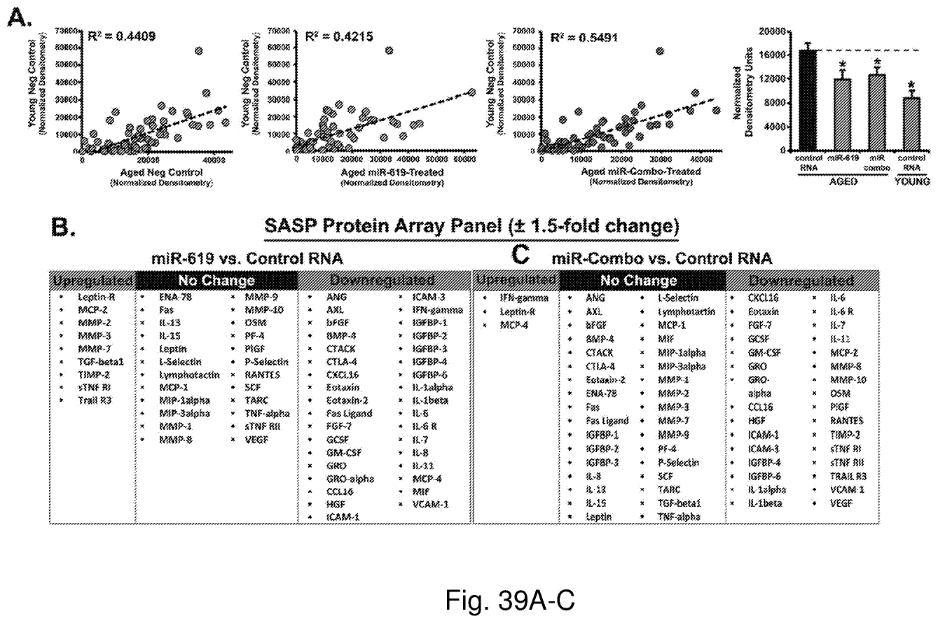

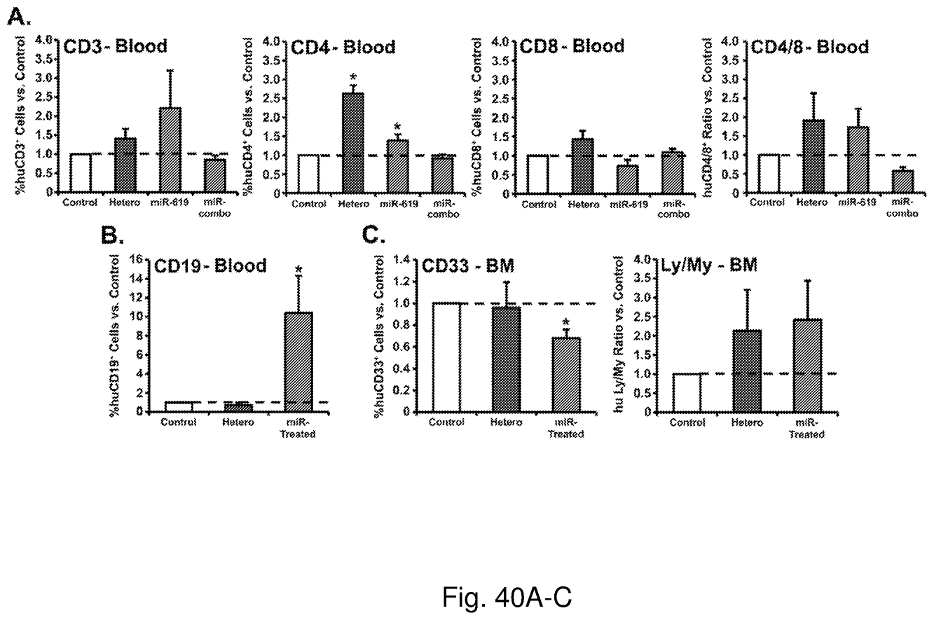

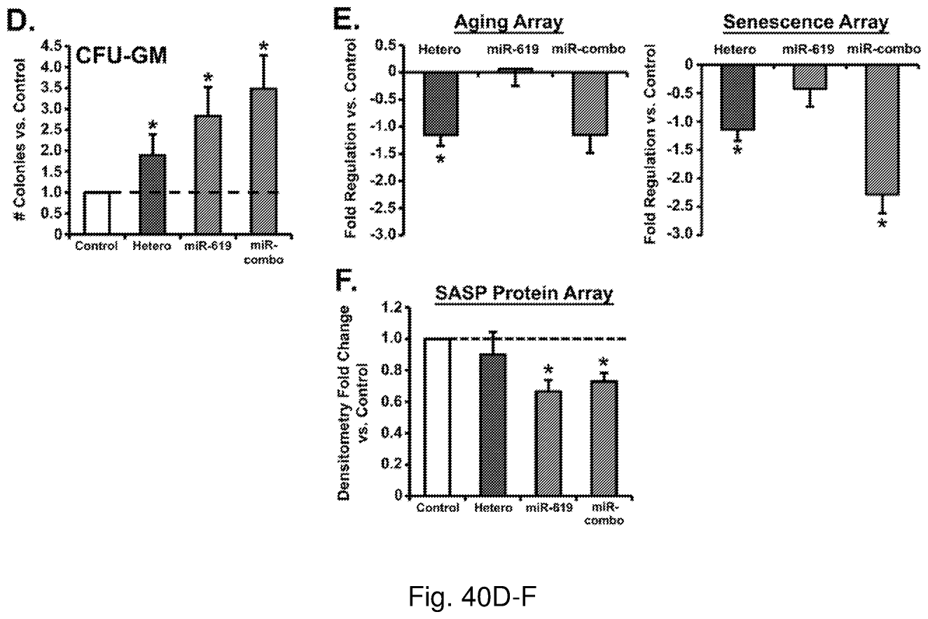

The findings from both animal studies illustrated that both heterochronic- and miRNA-based approaches to aged immune restoration was feasible. Thus, the results of both animal studies were indirectly studied by normalizing each study's treatment group(s) to the control group for key experimental readouts (FIG. 40). For the 1st animal study, heterochronic was normalized to isochronic data, and for the 2nd animal study, miRNA treatments were normalized to RNA control data.

For the blood T cell phenotypic panels (CD3+, CD4+, CD8+, CD4+/CD8+), similar results were observed among the heterochronic- (FIG. 40A) and miR-619-treated groups. However, the miRNA-treated groups showed significantly increased levels of CD19+ cells in blood (FIG. 40B), and significantly decreased levels of CD33+ cells in BM (FIG. 40C, left panel) compared to heterochronic treatment. No differences were observed for lymphoid/myeloid ratio (right panel). For the clonogenic assay, significant increases in clonogenicity for all treatment groups were observed, but treatment with miR-619 alone or in combination produced a more robust finding (FIG. 40D). Interestingly, for the gene array studies (FIG. 40E), the miR-combo treatment yielded the most dramatic downregulation of senescence-related genes (right panel) compared to either miR-619 alone or heterochronic treatment and was comparable to heterochronic for the aging array (left panel). Last, both miRNA treatments produced a significant decrease in SASF production (FIG. 40F) compared to heterochronic treatment.

Collectively, the findings from this comparative analysis suggest that treatment of autologous aged cells with either miRNA formulation yields a more robust immune and stem cell restoration compared to heterochronic culture and lends support to further develop these formulations in other relevant preclinical models of disease as an immune therapy.

### Discussion

Research of the past decade has provided a fundamental understanding of aging biology that has driven new and innovative approaches to extend organismal lifespan and healthspan. The utilization of young blood and factors present in the young systemic circulation to rejuvenate aged tissues, elegantly demonstrated by studies of heterochronic parabionts, is among these innovations. However, while young blood has been evidenced to contain factors that restore select tissue function, attempts to identify the restorative factors have been limited and identification of causal molecules refuted. Further, these initial findings were restricted to mouse blood, as only recently has a study of human UCB demonstrated a comparable effect in the CNS. To build on these limitations, the goal of these studies was to develop an adaptation of the heterochronic parabiosis model that would be immediately translational through utilization of blood from young and aged human donors. It was rationalized that young individuals with extremely healthy lifestyles should encompass high levels of restorative factors within their blood. A number of strict inclusion and exclusion criteria to select for good sleeping habits, diet and regular exercise were employed for young donor recruitment (Table 4). Selecting healthy aged donors was done in a diligent way, as it was desired to have any differential correlates to be associated with the temporal aspect of aging rather than age-related co-morbidities. This allowed for therapeutic factors that were uncovered to be applied in both preventative and interventional settings.

A facet of parabiotic studies that remained to be addressed was the effect of the young systemic circulation on the lymphohematopoietic system of the aged parabiont. The hematopoietic and immune systems are vital components of how organisms function. Blood cells perfuse most tissues of the body and serve local housekeeping and surveillance roles within the tissue microenvironment. The goal of these studies was to target the declining function of aging HSCs as a means to treat and delay the onset of age-related diseases. This approach modulated aging-related pathways to restore function to the aged HSC compartment. For the current investigation, it was important to develop a sophisticated animal model that could recapitulate the aging human lymphohematopoietic system and allow translational studies. This is believed to be the first study to engraft aged CD34+ cells for creation of humanized mice, as most approaches utilize young or primitive cells from UCB or fetal tissues. The aged huNSG model allowed the simulation of an adoptive cell therapy product, in which aged blood cells were restored ex vivo and then autologously transplanted into the aged individual. It was found that huNSG mice receiving adoptive restoration therapy exhibited significant increases in BM clonogenicity and lymphopoiesis compared to age-matched controls (FIG. 26). These findings are therapeutically-relevant, as many of the age-related increases in morbidity and mortality of the aging immune system can be attributed to HSC exhaustion and myeloid bias.

This study primarily utilized cells from MPB to establish heterochronic cultures. MPB comprises a heterogeneous population that is representative of both BM and the systemic circulation. It was rationalized that cultivation of such a complex cell matrix would simulate an ectopic microenvironment for paracrine communication among young and aged cells. Several unexpected observations were made from these culture studies. First, a partial role for young CD34+ cells was identified in the restorative mechanism (FIG. 24). This population encompasses HSCs, HPCs and EPCs, however other young immune or stromal populations likely contribute to the restorative phenotype, as CD34-depletion was not completely mitigating. Second, heterochronic restoration was sustainable. Restored aged cells propagated restoration in culture (FIG. 24) and in huNSG mice (FIG. 26). The latter conclusion was supported by gene expression studies of purified human cells from restored mouse BM. Here, it could be argued that decreases in senescence- and aging-related gene expression occur primarily in cells from the original graft, since the second transplant comprised a relatively low cell dose compared to the number of cells that would be the product of endogenous human hematopoiesis. Further, no spike in human chimerism was observed following the second transplant to suggest massive expansion in the BM compartment (FIG. 25).

Investigation into the paracrine mechanism of heterochronic restoration revealed a causal role for exosomes and miRNAs (FIGS. 31 and 33). Exosomes contain a subset of proteins, lipids and nucleic acids that are derived from the parent cell and which are shuttled between cells. miRNAs taken up by target cells can change target cell behavior by classical miRNA-induced silencing of target mRNAs. This form of intercellular communication is involved in numerous physiological processes, including immune regulation. Exosomes have gained substantial traction as a therapeutic for a number of indications ranging from cancer to immune-related diseases to regenerative medicine. A mechanistic role was attributed to exosomes and miRNA based on findings from studies of purified young exosomes and inhibition of exosomal miRNA biogenesis, respectively (FIG. 31). There is a distinct possibility that other soluble factors beyond exosomes contribute to restoration, since proteins found in UCB and young mouse plasma, such as TIMP2, have restored cognitive function in aged mice. Further, exosomes also house other species of non-coding RNA, such as long non-coding RNAs (lncRNAs) and circular RNAs, that could be involved in restoration. miR-619-5p was identified as a stand-alone restorative factor found within young exosomes that could induce restoration independently of young cells (FIG. 33). While little is known regarding physiological function, miR-619 may be a unique miRNA (umiRNA). umiRNAs are known to have hundreds of target genes and bind to mRNAs with high affinity, thus the suggested downstream effect of miR-619 would be broad-acting and consistent with gene expression changes observed during restoration. In silico prediction of pathways targeted by young exosomal miRNA included master regulators of cell senescence, such p53, p21 and p16INK4a (FIG. 35). Network analysis of restorative miRNA (FIG. 33) in the context of these pathways predicted 5 direct targets in aged cells, of which PAX5 and PPM1F were validated. The PAX5 gene encodes the B-cell lineage specific activator protein (BSAP) that is expressed at early, but not late stages of B-cell differentiation, while the PPM1F gene encodes a member of the PP2C family of Ser/Thr protein phosphatases that are negative regulators of the cell stress response pathways. FIG. 41 summarizes the downstream pathways engaged by young exosomal miRNA following PAX5 and PPM1F mRNA target engagement in the aged cell. Both PAX5 and PPM1F directly and indirectly regulate pathways involved in cellular senescence, respectively. PAX 5 can directly activate p53 and p21 signaling, while PPM1F can activate similar pathways indirectly through CaMKIIγ.

A second huNSG study was performed, this time utilizing two separate formulations of candidate miRNAs to induce aged cell restoration prior to autologous transplant (FIG. 36). The first formulation contained a high dose of miR-619 alone (90 nmol), while the second contained a combinatorial formulation including miR-619, miR-1303 and miR-4497 transfected at 30 nmol each to yield an equal total RNA dose as the standalone treatment. Interestingly, with the miRNA treatments it was again observed therapeutic benefit as defined by significant increases in BM clonogenicity and lymphopoiesis, as well as decreases in aging- and senescence-related gene and protein expression. When indirect comparative analyses were performed among the heterochronic- vs. miRNA-based treatments (FIG. 40), a similar effect on T cell phenotype was observed; however, the miRNA-treatments were more efficacious in boosting B cell production, decreasing myeloid output and increasing clonogenicity. Further, the miRNA combination treatment of miR-619, miR-1303 and miR-4497 proved to be efficacious in improving stem cell function and decreasing aging-related gene and protein expression. These findings provide support to further develop this combination as a novel therapeutic.

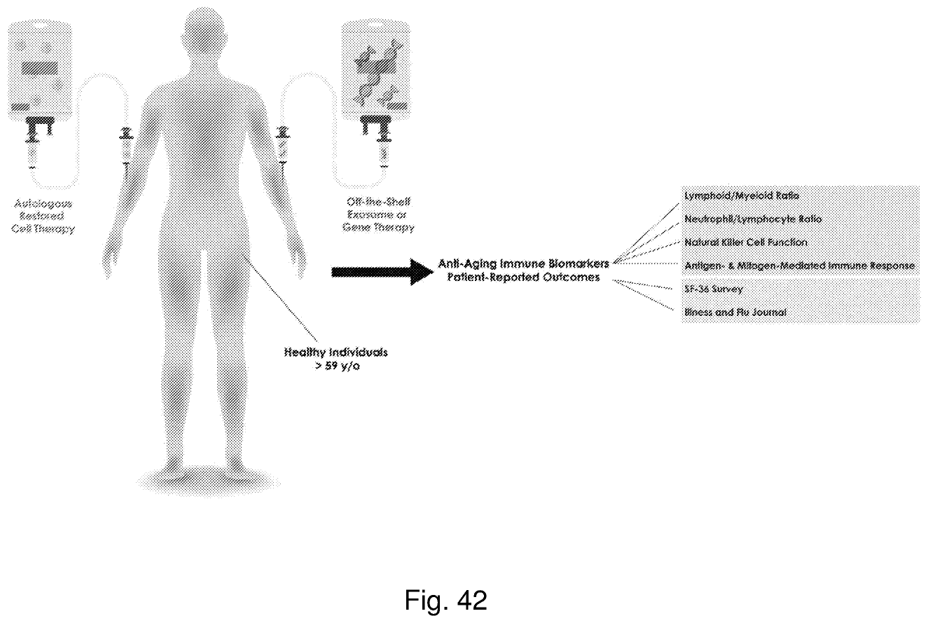

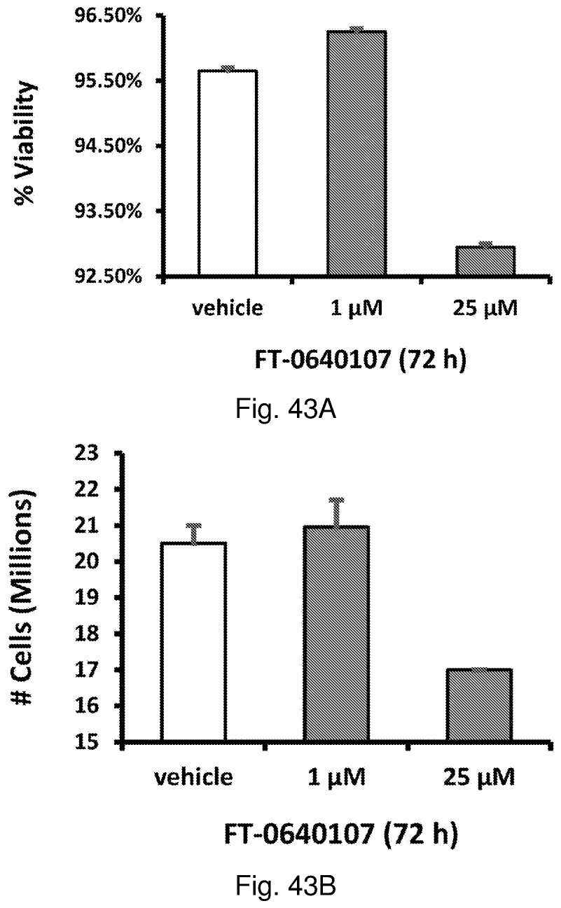

These studies highlight at least 3 potential approaches that could be implemented clinically: (1) an adoptive, autologous cell product derived from heterochronic cultivation; (2) similar as 1 but replacing young cells with a miRNA combinatorial formulation to induce restoration; or (3) a pharmacological inhibitor of PAX5, PPM1F or a downstream component of their signaling pathways. It is shown that the disclosed model is restorative for aged cells harvested from healthy donors, thus development of these approaches as a preventative therapy to enhance endogenous stem cell and immune function before the onset of disease is logical (FIG. 42). Further, there is also the exciting possibility of applying these approaches as an interventional therapy for aged patients afflicted with cancer or infectious disease, as either an autologous cell-based or cell-free restoration therapy. Existing immuno-oncology (I-O) biological therapies, such as checkpoint inhibitors and bispecific antibodies, and cell-based therapies, such as chimeric antigen receptor (CAR) T cells, utilize a targeted approach to coax the immune system to recognize and attack cancer cells. A restorative product could be administered in combination with these therapies to bolster the endogenous immune system in advance of the targeted response, an approach shown recently to be effective in mice. Additional studies will test patient blood samples to evaluate and demonstrate that the disclosed approach enhances tumor clearance either alone or in combination with existing I-O drugs.

Studies have relied on CD34-rich sources such as MPB and UCB. However, the translating these data to non-mobilized blood may be attractive and possible, since mobilization is costly and potentially impractical for cancer patients. Beyond blood, determining whether this approach could functionally restore other aging tissues, such as stromal vascular fraction (SVF) harvested from adipose is attractive. SVF is a rich source of preadipocytes, adipose-derived stem cells (ADSCs), EPCs and other resident immune cells, which can be utilized clinically for various orthopedic applications. Since restorative factors directly target ubiquitous pathways related to aging and senescence, this approach should be therapeutic for a number of aging tissues. Extrapolation of these findings to these other aging systems is possible in view of these studies.

### Example 8: Small Molecules Inhibit Proteins Having Roles in Cellular Dysfunction

Aged TNCs (20×106 cells per sample) were cultured with CaMKP inhibitor (FT-0640107 shown below) at the following concentrations 25 uM, 10 uM, 5 uM, 1 uM & 0 (vehicle). After 48 and 72 hours, cells were harvested, counted using Turk's solution and percent viability assessed using Trypan blue exclusion.

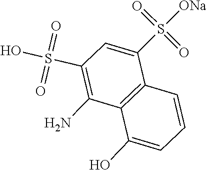

At 72 hours following treatment with the CaMPK inhibitor, FT-0640107, or vehicle, cells were harvested, counted and viability measured using Trypan blue exclusion. Cells treated with 1 μM FT-0640107 for 72 hours demonstrated an average cell viability of 96.3%, whereas treatment with vehicle alone yielded an average cell viability of 95.7% and cells treated with a high dose of 25 μM of the compound exhibited a viability of 93%. Further, cells treated with 1 μM FT-0640107 for 72 hours yielded an increase in cell number compared to both vehicle control and high dose treatment (25 μM). The data were observed for donor A04, with no significant difference observed for donor A03 at 48 or 72 hour timepoints. These findings demonstrate that pharmacological inhibition of CaMPK with FT-0640107 can exert a therapeutic benefit on aged mobilized blood cells by improving cell viability and survival in culture.

### Example 9: Sample and Techniques

Peripheral blood mononuclear cells were collected from subjects having undergone a Stage B preparation involving the administration of the mobilizer NEUPOGEN® (filagrastim). For the following experiments, peripheral blood mononuclear cells (1×5 mL vial per subject) were found to contain the following number of nucleated cells (Table 7).

TABLE 7
Nucleated cell number count in subjects

| Subject | Approximate Age | Sex | Nucleated Cell Number |
|---|---|---|---|
| R1 | 70 | Male | 1.8 × 10⁸ |
| R2 | 70 | Male | 2.7 × 10⁸ |
| R3 | 60 | Male | 2.2 × 10⁸ |
| D1 | 26 | Male | 3.3 × 10⁸ |
| D2 | 30 | Male | 1.8 × 10⁸ |
| D3 | 28 | Female | 1.9 × 10⁸ |

The cells following harvest were cryopreserved and upon thawing were determined by trypan blue dye exclusion and flow cytometry to be greater than 95%.

The percentages of stem cell, progenitor cell and mature cell populations were determined by flow cytometry. Table 8 shows the average percentage of hematopoietic stem cells (HSCs) and early hematopoietic progenitor cells (HPCs) to be ˜0.5% and 1.0% respectively of the total mobilized cell collection.

TABLE 8
Cell type percentages in PBMC samples from subjects

| | | Hema- | Hema- | | |
|---|---|---|---|---|---|
| | | topoietic | topoietic | | |
| | | Stem | Progenitor | | |
| | Hema- | Cells | Cells | | |
| | topoietic | CD34⁺ | CD34⁺ | T Cells | NK Cells |
| | Cells CD45⁺ | CD38⁻ | CD38⁺ | CD3⁺ | CD56⁺ |
| % Total | | | | | |
| Cell | | | | | |
| Population | | | | | |
| R1 | 64.4 | 0.5 | 0.3 | 39.6 | 4.2 |
| R2 | 59.9 | 0.4 | 1.6 | 29.7 | 7.5 |
| R3 | 86.3 | 0.4 | 0.3 | 56.7 | 2.9 |
| D1 | 64.0 | 1.0 | 1.5 | 32.4 | 5.7 |
| D2 | 59.9 | 0.3 | 0.8 | 36.0 | 4.1 |
| D3 | 59.0 | 4.1 | 1.0 | 10.5 | 5.5 |
| % Hema- | | | | | |
| topoietic | | | | | |
| (CD45⁺) | | | | | |
| Population | | | | | |
| R1 | 100 | 0.7 | 0.4 | 61.5 | 6.5 |
| R2 | 100 | 0.7 | 2.8 | 49.5 | 12.5 |
| R3 | 100 | 0.4 | 0.4 | 65.7 | 3.4 |
| D1 | 100 | 1.5 | 2.4 | 50.5 | 8.9 |
| D2 | 100 | 0.5 | 1.3 | 60.1 | 6.9 |
| D3 | 100 | 7.0 | 1.7 | 51.7 | 9.3 |

| | | |
|---|---|---|
| | Mesen- | |
| Non- | chymal | |
| Hema- | Stem Cells | Endothelial |
| topoietic | CD29⁺ | Progenitor Cells |
| Cells | CD44⁺ | CD31⁺ |
| CD45⁻ | CD105⁺ | CD105⁺ |

| | | | |
|---|---|---|---|
| % Total Cell | | | |
| Population | | | |
| R1 | 17.9 | 0.11 | 0.06 |
| R2 | 38.7 | 0.91 | 0.79 |
| R3 | 11.1 | 0.22 | 0.18 |
| D1 | 33.4 | 0.52 | 0.44 |
| D2 | 26.7 | 0.69 | 0.65 |
| D3 | 40.4 | 0.63 | 0.46 |
| % Non- | | | |
| Hematopoietic (CD45⁻ | | | |
| Population) | | | |
| R1 | 100 | 0.60 | 0.35 |
| R2 | 100 | 2.34 | 2.05 |
| R3 | 100 | 1.94 | 1.58 |
| D1 | 100 | 1.57 | 1.32 |
| D2 | 100 | 2.57 | 2.43 |
| D3 | 100 | 1.56 | 1.14 |

Receiver cell samples (i.e., R1, R2, and R3) were individually paired with donor cell samples (i.e., D1, D2, and D3) and the pairs co-cultured in a transwell culture cell for four weeks. The morphology of the cells was studied at 2 weeks into the co-culture and at 4 weeks of co-culture. Gene expression arrays, protein arrays, and telomere length experiments compared freshly defrosted cells from the collection tubes (referred to as the baseline donor cell sample or baseline receiver cell sample) with cells at the 4-week study endpoint (referred to as restored cell samples).

At the midpoint of the co-culture (i.e., 2 weeks) baseline receiver cell samples displayed morphologies consistent with low viability. Restored cell samples displayed a robust cellular morphology that included colony formation.

### Example 10

Protein array analyses were carried out using conditioned media from the baseline donor cell sample or baseline receiver cell sample. The conditioned media was mixed with like cellular protein extracts and applied to the custom-designed arrays which consisted of antibody probes for 68 factors linked to cellular aging and senescence, collectively referred to as the senescence-associated secretory factors (SASF). Quantitative PCR gene array analyses were carried out by extracting RNA from the baseline samples or the co-cultured donor and receiver samples. The data presented represents the average metric determined for either the baseline donor cell sample, the baseline receiver cell sample, or the restored cell sample.

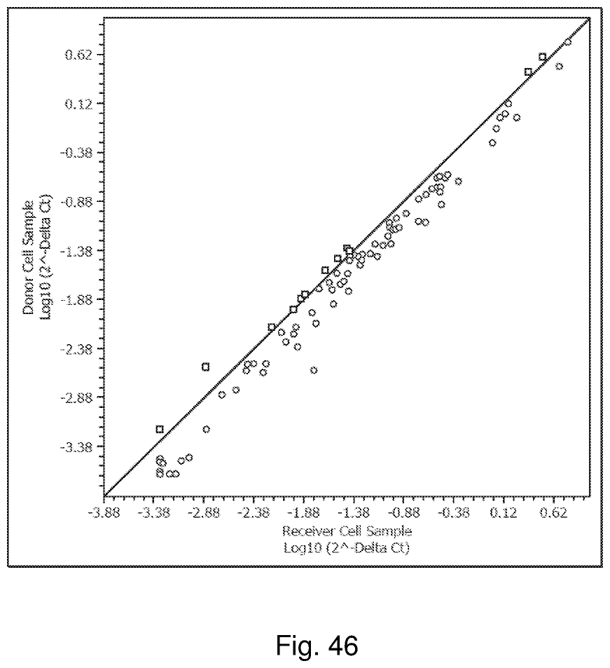

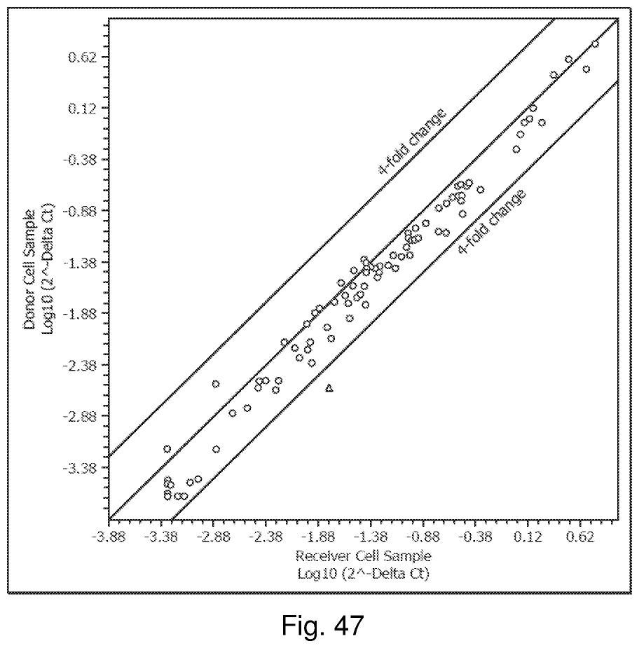

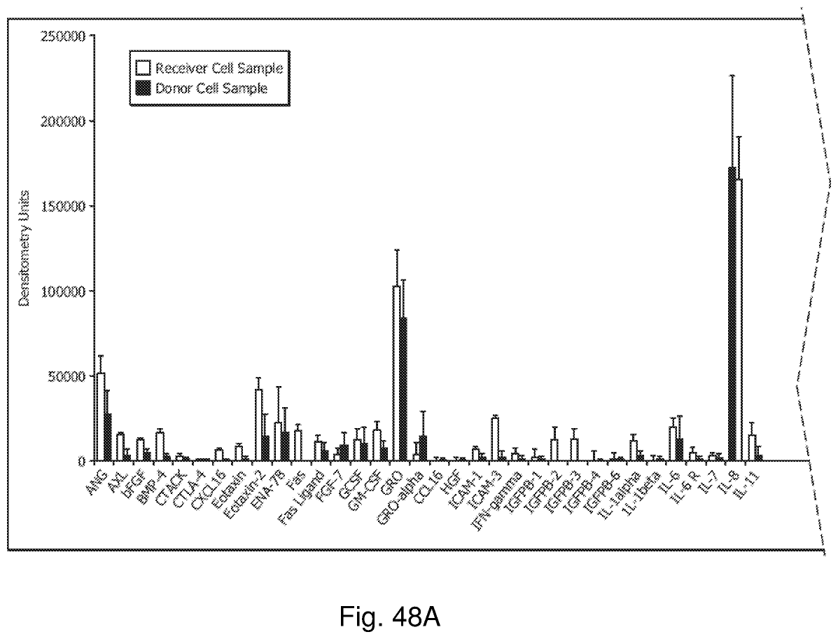

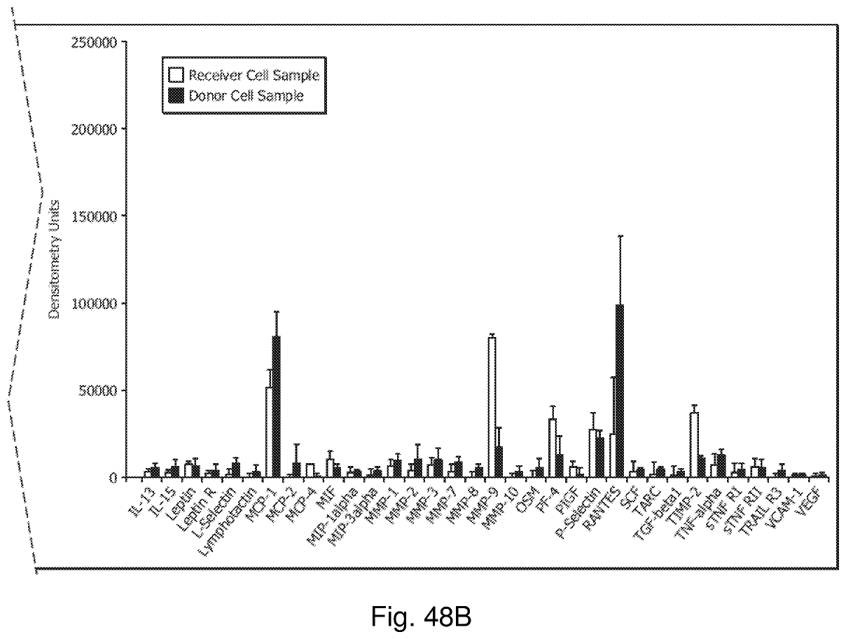

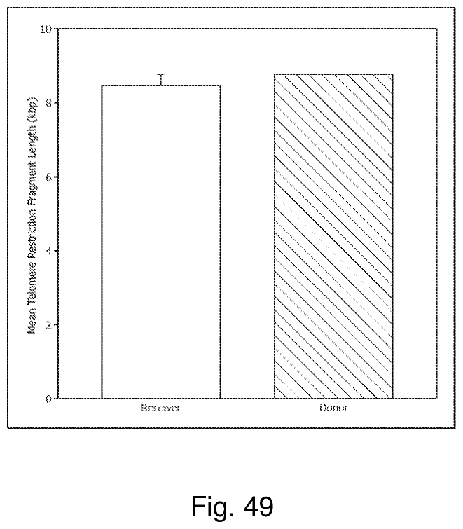

The results of the gene array analyses demonstrated that there was less than a 2-fold difference in the majority of senescence-related genes for the baseline donor cell samples and the baseline receiver cell samples, FIGS. 46 and 47. In all of the Figures presented as plots of the gene expression analysis, the squares represent genes which are expressed at a lower level following the cellular restoration, triangles represent genes expressed at a higher level following restoration and circles represent genes whose expression level was determined to be substantially similar to the expression level prior to cellular restoration. The designation of “substantially similar” is qualitative and reflects the close proximity of the value to the line. The data suggests the techniques disclosed herein for the mobilization and collection of donor cell samples and receiver cell samples select for non-senescent cells. The results of the protein-based arrays, which assessed levels of senescence-related factors produced either within the cells or released into the culture media, similarly displayed little difference between the senescence-related factors among the donor cell sample and receiver cell sample, FIG. 48. The mean telomere length between the donor cell samples and receiver cell samples were not found to be significantly different (FIG. 49).

The results demonstrate that the gene expression profiles and protein expression profiles of the donor cell samples and receiver cell samples were similar despite the difference in age of the subjects. Further, despite the difference in age between the donor subjects and receiver subjects, the cells had similar telomere lengths.

### Example 11

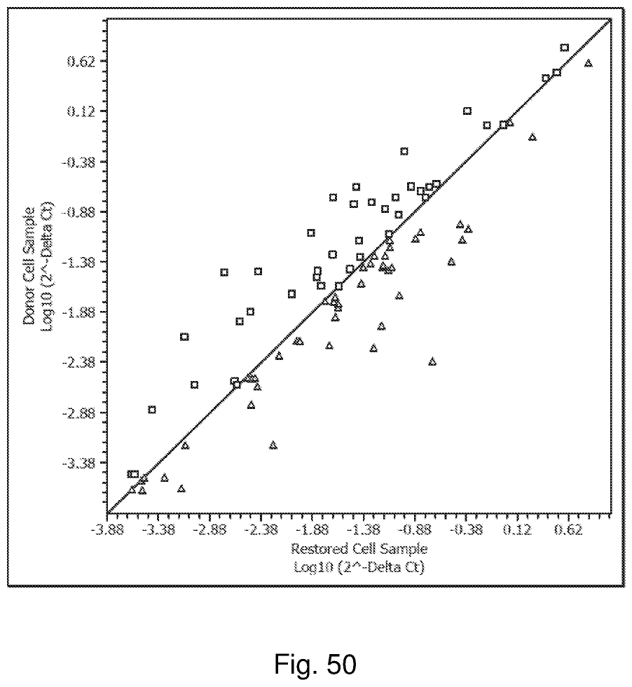

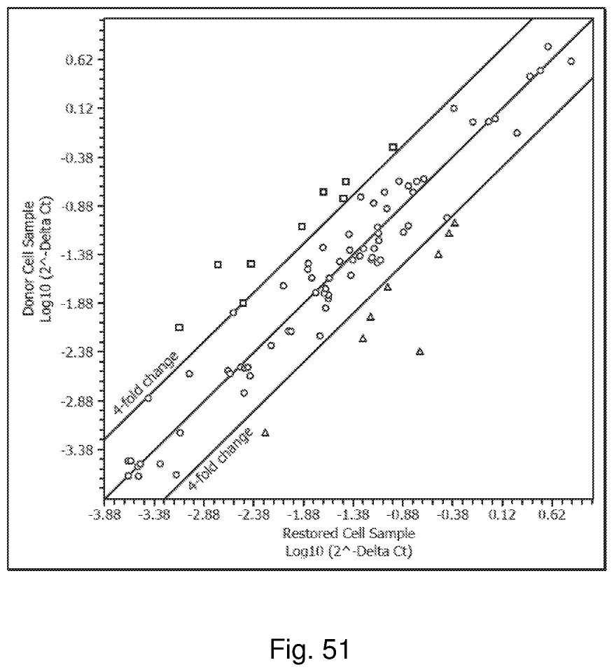

The restored cell samples were investigated using the gene and protein arrays of Example 2 and are shown in FIGS. 50 and 51, respectively. For each of the restored cell samples investigated, approximately half of the examined genes (as designated by the “Xs” on the Figure) were expressed at a lower level in the restored cell samples when compared to the baseline donor cell sample. The genes that were expressed at a lower level were genes associated with improving cellular function and decreasing the extent of cellular senescence and aging. The data suggests the gene expression profile of the restored cell samples were altered by the transwell restoration and more closely approximated that of the baseline donor cell sample than that of the baseline receiver cell sample. The data demonstrate the restore cell sample exhibited a decreased expression of senescence-related genes of receiver cells and/or cell types compared to the receiver cell sample, wherein senescence-related genes are defined as the RBGEP, by quantitative polymerase chain reaction as shown in FIGS. 50 and 51.

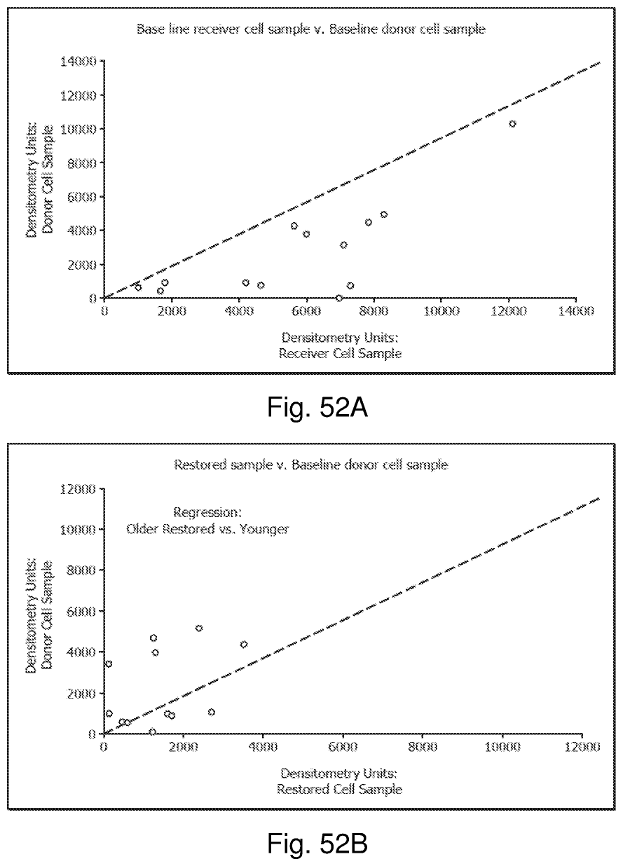

Further, examination of the clustering analyses revealed a subset of genes (designated gene set A) whose expression was consistently elevated in the baseline receiver cell sample but whose expression was reduced in the restored cell sample. The expression of Gene Set A in the restored cell sample was reduced to levels comparable to those observed in the baseline donor cell sample. Similarly, as shown in FIG. 52A, stratification of the protein arrays identified 13 factors that showed a similar elevated trend in the baseline receiver cell samples when compared to that of the baseline donor cell samples. Likewise, as shown in FIG. 52B, the restored cell samples exhibit a level of expression of the identified 13 factors comparable to that observed in the baseline donor samples. These findings demonstrate a methodology for monitoring the restoration of the receiver cell sample by gene and protein array analyses. Further, these data demonstrate the restored cell sample exhibited a decreased expression of senescence-associated secretory factors compared to the receiver cell sample at baseline, wherein senescence-associated secretory factors are defined in Table 1, as measured by antibody array, as in FIGS. 52, 53 and 54, or enzyme-linked immunosorbant assay.

### Example 12

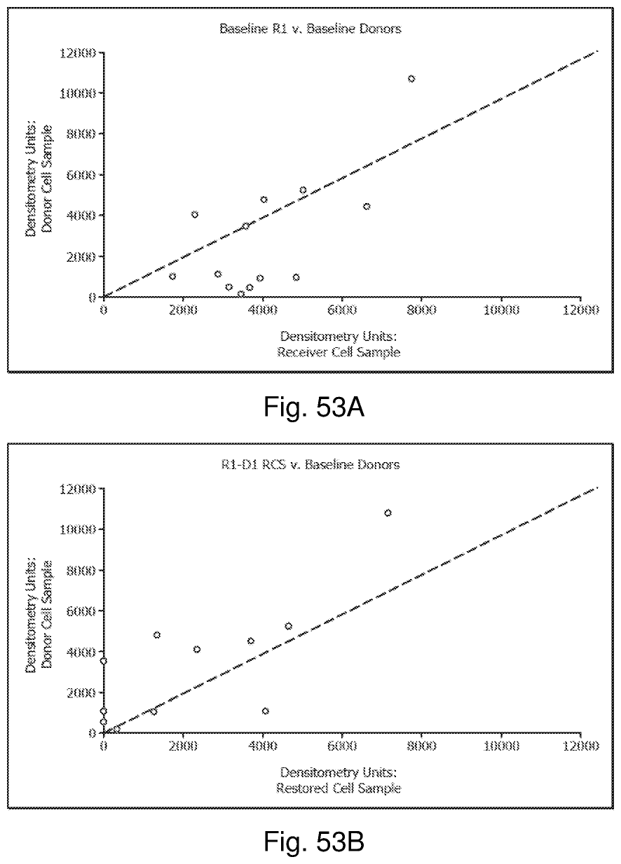

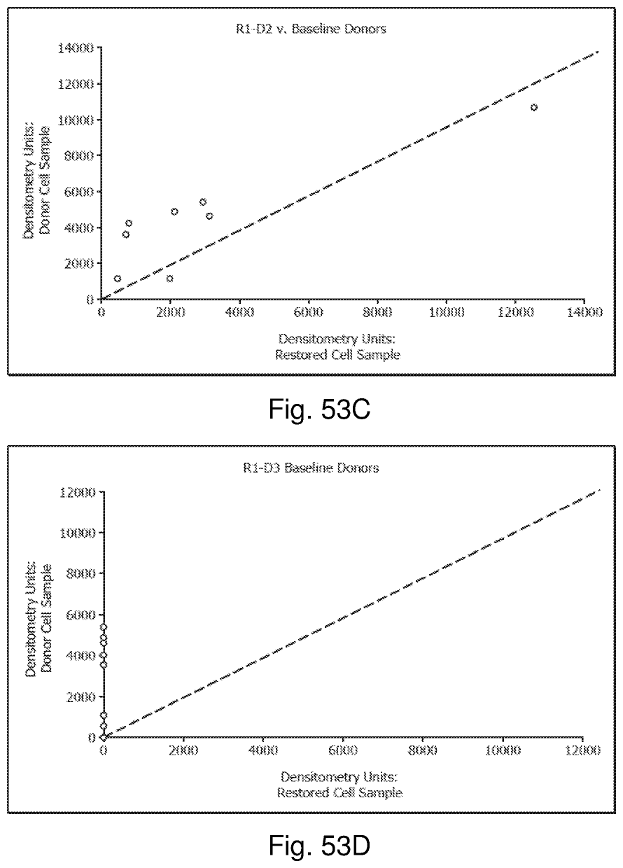

Gene and protein arrays of restored cell samples for individual pairs of baseline donor cell samples and receiver cell samples were investigated. Specifically, receiver cell sample R1 was co-cultured in a transwell experiment with donor cell samples from D1 (FIG. 53B), D2 (FIG. 53C), and D3 (FIG. 53D) respectively. Hierarchical clustergrams showed that all of the genes investigated are elevated in the receiver cell sample (FIG. 53A) while these same genes are expressed at low to modest levels in D1, D2 and D3. The restored cell sample was found to have a gene expression comparable to that of the level of expression observed for D1, D2, and D3. Experiments carried out using receiver cell samples R2 or R3 with donor cell samples D1, D2, or D3 exhibited similar results.

### Example 13

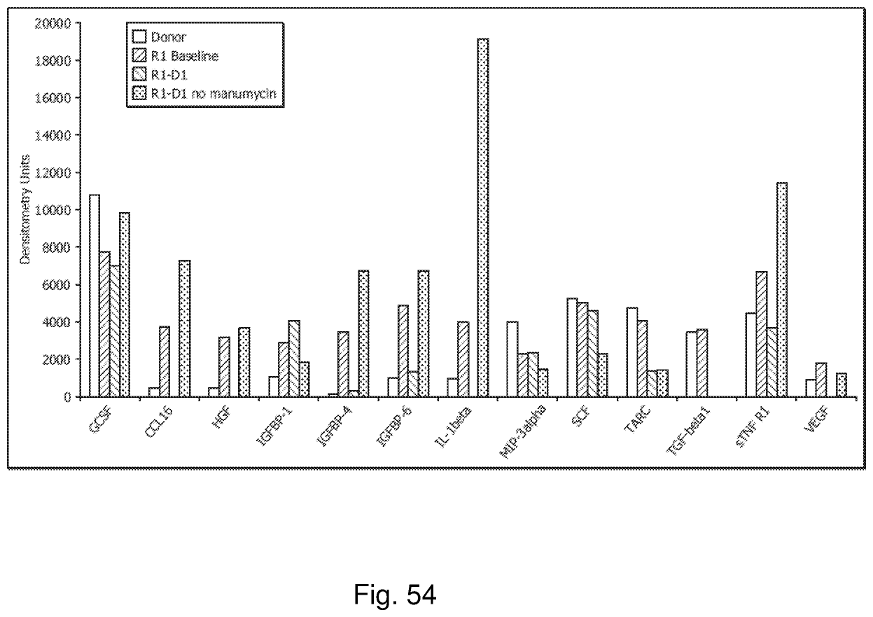

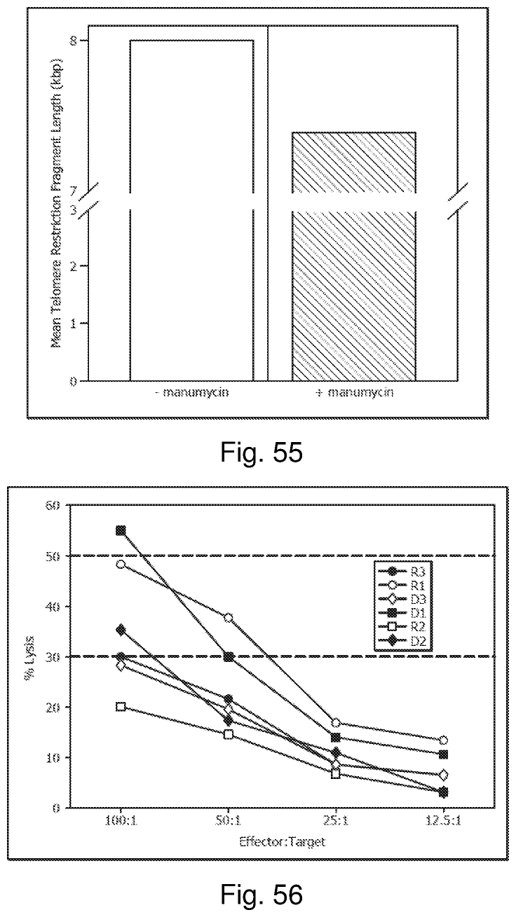

The nature of the soluble particles passing through the permeable membrane in the transwell co-culture experiments was investigated. Specifically, transwell co-culture experiments were carried out in the presence or absence of manumycin. Manumycin, N-[(1S,5S,6R)-5-hydroxy-5-[(1E,3E,5E)-7-[(2-hydroxy-5-oxo-1-cyclopenten-1-yl)amino]-7-oxo-1,3,5-heptatrien-1-yl]-2-oxo-7-oxabicyclo[4.1.0]hept-3-en-3-yl]-2E,4E,6R-trimethyl,2,4-decadienamide, is an antibiotic that acts a potent and selective farnesytransferase inhibitor. Manumycin is also known to inhibit the release of exosomes. Restored cells co-cultured in the presence of 5 μM manumycin displayed less robust morphology than restored cells cultured in the absence of manumycin. Gene expression analysis of restored cell samples co-cultured in the presence of manumycin did not display a change in expression levels similar to those observed in the absence of manumycin. In contrast, protein expression analyses of restored cell samples co-cultured in the presence of manumycin found elevated levels of all proteins investigated when compared to the proteins levels for restored cell samples co-cultured in the absence of manumycin (FIG. 54). The data suggests the role of exosome/microvesicles in mediating the disclosed cellular restoration process. Further support for the role of exosome/microvesicles in mediating the disclosed cellular restoration process is shown in FIG. 55. FIG. 55 displays the telomere length for a restored cell sample co-cultured in the presence or absence of manumycin. The telomere length in the presence of manumycin is decreased suggesting exosome/microvesicles play a role in the restoration process.

### Example 14

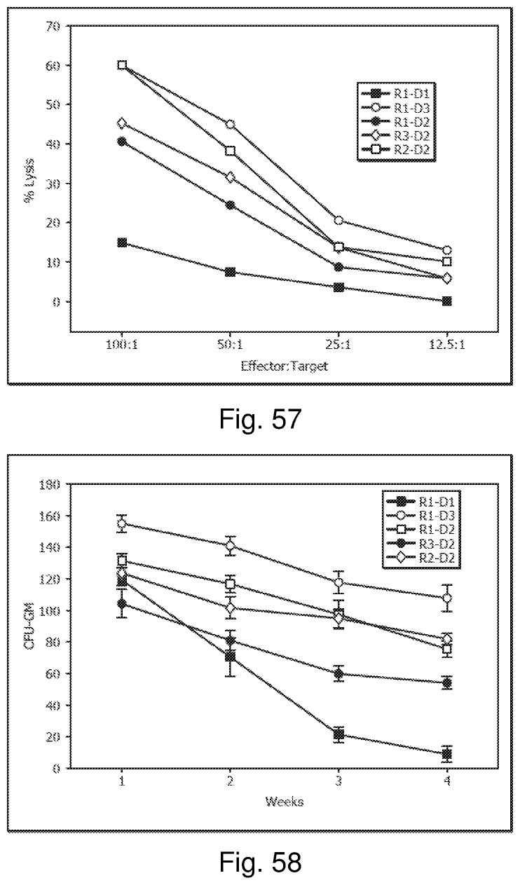

The innate immune function of the baseline donor cell samples and receiver cell samples were evaluated using a natural killer cell assay (FIG. 56). The assay was also performed on restored samples (FIG. 57). The restored cell samples are identified by the receiver cell sample-donor cell sample that were contacted, for example, receiver cell sample 3 and donor cell sample D2 are listed as R3-D2. The data demonstrate that the restored cell samples R1-D3, R1-D2, R3-D2, and R2-D2 maintained proper immune function while restored cell sample R1-D1 had decreased immune function. The data illustrate that for D2 and D3 the restored cell samples are characterized by an improvement in cellular immune function as quantified by natural killer cell cytotoxicity assay, as illustrated in FIGS. 56 and 33, and/or T-cell mitogen response assay.

The hematopoietic function of the baseline donor cell samples and receiver cell samples were evaluated using a clonogenic assay (FIG. 58). The data demonstrate that the restored cell samples R1-D3, R1-D2, R3-D2, and R2-D2 maintained proper hematopoietic function while restored cell sample R1-D1 had decreased hematopoietic function. The data illustrate that the restored cell sample is characterized by an improvement in cellular hematopoietic function as quantified by hematopoietic stem cell clonogenic assay (FIG. 58).

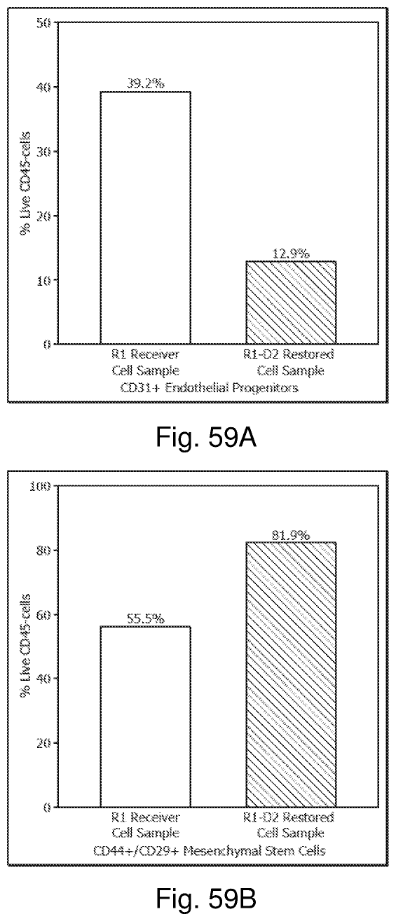

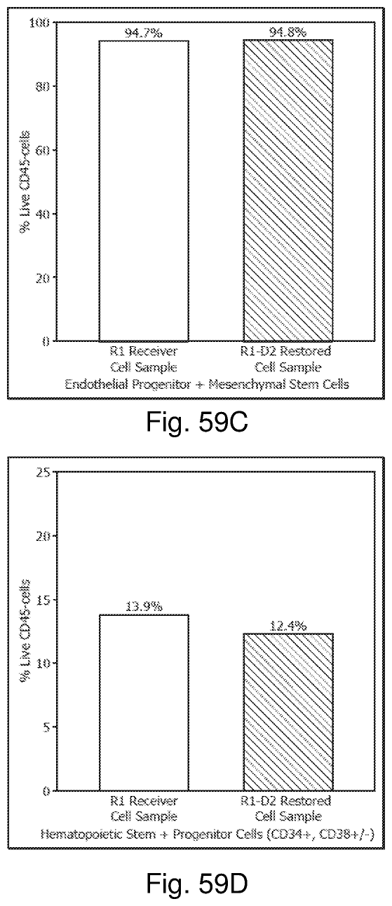

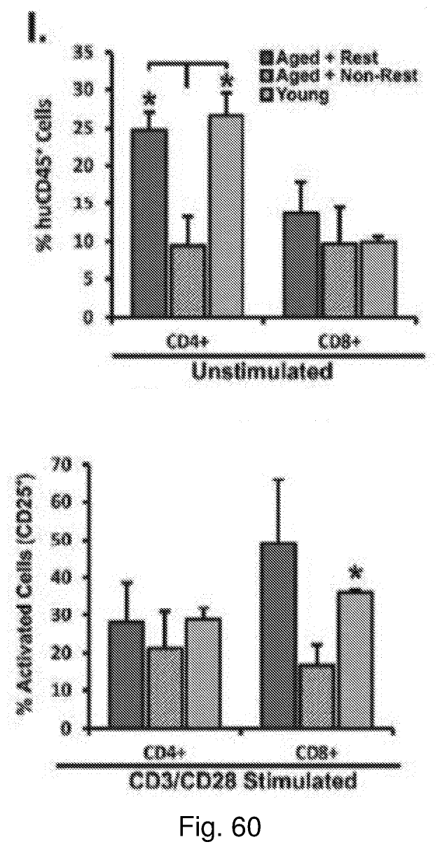

Cell population analyses were performed by flow cytometry to investigate whether the original distribution of cell types in the receiver cell sample, R1, was altered by the 4-week restoration process with donor cell sample D2. The results, shown in FIGS. 59A-D, demonstrated that although there was some loss in the percentage of endothelial progenitor cells (FIG. 59A), there was an expansion of the mesenchymal stem cell compartment (FIG. 59B). As shown in FIG. 59C, a combination of the percentages of non-hematopoietic cells (i.e., from FIGS. 59A and B) indicated that the total percentages of these populations were maintained during the restoration process. Similarly, as shown in FIG. 59D, the percentages of hematopoietic stem and progenitor cells were insignificantly changed. These data demonstrate that the restored composition prepared by the methodologies disclosed herein is characterized by a lack of change in the percentage of hematopoietic stem cells, hematopoietic progenitor cells, mesenchymal stem cells and endothelial progenitor cells, herein termed the “stem cell pool”, in receiver cells after cellular restoration compared to receiver samples at baseline, as observed in FIG. 59, by flow cytometry.

### Example 15

The following data establishes that the restored compositions and/or other agents disclosed herein (e.g., exosomes, RNAi(s), small molecules, etc.) may be used in methods to restore immune function in aged patients. The following study describes the effect of infusing restored compositions into aged individuals, herein termed AR-100. Tables 9-11 show inclusion/exclusion criteria (Table 9) and patient demographics and dosing (Table 10 and Table 11).

TABLE 9
Inclusion/Exclusion criteria.

| Donor | Inclusion Criteria | Exclusion Criteria |
|---|---|---|
| Aged | ≥59 years old | History of blood borne |
| | Healthy | tumor Contrain- |
| | | dication to G-CSF |
| Young | 18-29 years old | Abnormal BMI |
| | Normal BMI (18.5-25) | (underweight, |
| | At least 5 days/week of | overweight, obese) |
| | moderate to strenuous | Moderate to heavy regular |
| | exercise (minimum of 30 min) | alcohol consumption |
| | Successful completion of | Prior cancer diagnosis |
| | physical examination | HIV, HPV, HBV or |
| | Non-smoker | HCV positive test |
| | Weigh at least 120 lbs | |

TABLE 10
Patients treated.

| | | | | Most Recent | Last Measured Test |
|---|---|---|---|---|---|
| Patient | | | Date of | Testing | Post-Treatment |
| ID | Sex | Age | Treatment | Endpoint | (weeks) |
| PT-001 | M | 78 | Dec. 15, 2017 | Jan. 16, 2019 | 56 |
| PT-002 | M | 67 | Apr. 6, 2018 | Jan. 30, 2019 | 42 |
| PT-003 | M | 75 | Apr. 6, 2018 | Oct. 3, 2018 | 26 |
| PT-008 | M | 68 | Feb. 4, 2019 | Nov. 28, 2018 | N/A |
| PT-006 | M | 79 | Feb. 28, 2019 | Jan. 22, 2019 | N/A |

TABLE 11
Patient cell culture seeding and dosing.

| Patient | # Cells Seeded in | % Viability | # Cells | % Viability |
|---|---|---|---|---|
| ID | Culture (× 10⁶) | (Seeded) | Infused (× 10⁶) | (Infused) |
| PT-001 | 2300 | 74 | 362 | 68 |
| PT-002 | 2000 | 79 | 800 | 71 |
| PT-003 | 2500 | 81 | 595 | 78 |
| PT-008 | 2195 | 98 | 585 | 94 |
| PT-006 | 1800 | 89 | 563 | 75 |

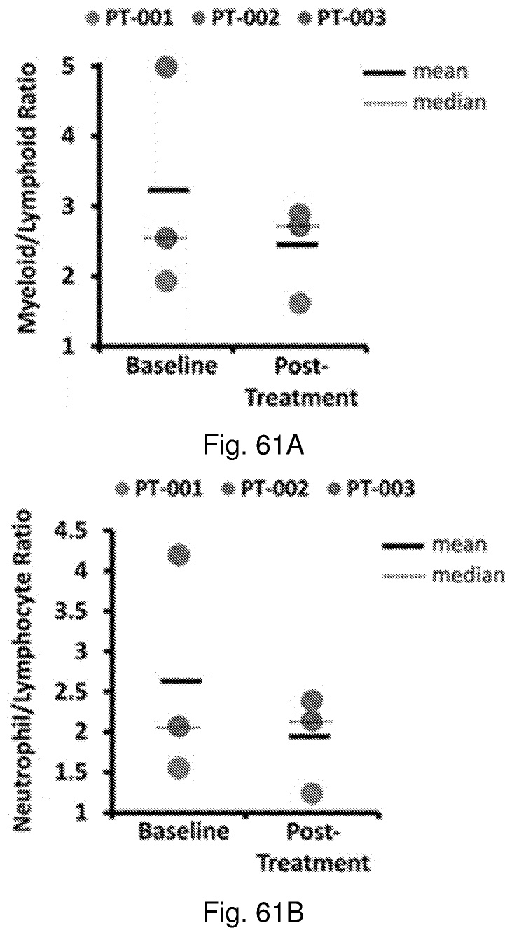

A hallmark of an aging immune system is decreased production of cancer-fighting and infection-fighting lymphoid cells, such as lymphocytes. Another hallmark is increased production of pro-inflammatory myeloid cells, such as neutrophils. By measuring the ratio of myeloid to lymphoid and neutrophil to lymphocyte cell output in the blood, an assessment of immune competence can be shown. FIGS. 61A and 61B, below, show results obtained for cell phenotyping and safety assessments. Patients treated with AR-100 showed decreased myeloid to lymphoid ratios (FIG. 61A) and decreased neutrophil to lymphoid ratios (FIG. 61B). These improved ratios should lead to improvements in treating disease states as disclosed elsewhere herein, including age-related cellular dysfunction in these patients.

Some embodiments, provide methods (e.g., through exposure to restored compositions, RNAi(s), exosomes, small molecule drugs, etc.) for decreasing a patient's inflammatory cell to lymphoid cell ratio, myeloid to lymphoid ratio, and/or neutrophil to lymphoid ratio. In some embodiments, the inflammatory cell to lymphoid cell ratio, myeloid to lymphoid ratio, and/or neutrophil to lymphoid ratio, is decreased by equal to or less than about: 1%, 2%, 3%, 4%, 5%, 10%, 20%, 25%, 30%, 40%, 50%, 60%, 70%, 75%, 80%, 90%, 100%, 200%, 300%, 400%, 500% or ranges including and/or spanning the aforementioned values.

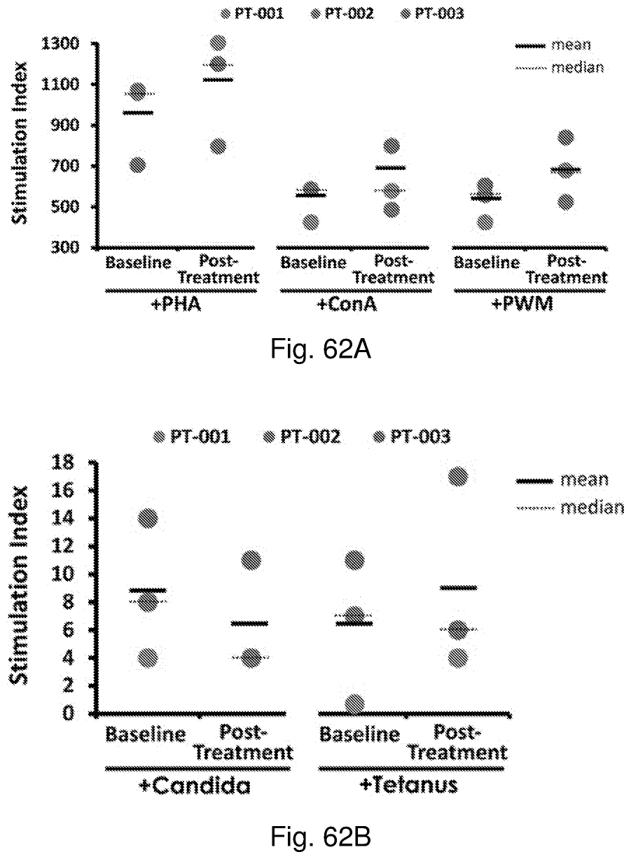

FIGS. 62A and 62B show functional immune profiling of peripheral blood mononuclear cells (PBMC). PMBC were stimulated with the T-cell mitogens, Phytohemaglutinin (PHA), Concavalin A (ConA), or the T and B cell mitogen, Pokeweed Mitogen (PWM) (FIG. 62A). Blood cells were also stimulated with the Candida albicans or Tetanus toxoid antigens (FIG. 62B). On average, patients treated with AR-100 demonstrated a strong proliferative response following mitogen stimulation and/or antigen stimulation. These findings suggest sustainable increases in innate and/or cell-mediated immunity post-treatment for all patients. These changes should lead to improvements in treating disease states as disclosed elsewhere herein, including age-related cellular dysfunction in these patients.

Some embodiments, provide methods (e.g., through exposure to restored compositions, RNAi(s), exosomes, small molecule drugs, etc.) for increasing mitogen-induced immune response, antigen-induced immune response, PBMC proliferation, or combinations thereof. In some embodiments, mitogen-induced immune response, antigen-induced immune response, and/or PBMC proliferation, is increased by equal to or at least about: 1%, 2%, 3%, 4%, 5%, 10%, 20%, 25%, 30%, 40%, 50%, 60%, 70%, 75%, 80%, 90%, 100%, 200%, 300%, 400%, 500%, or ranges including and/or spanning the aforementioned values.

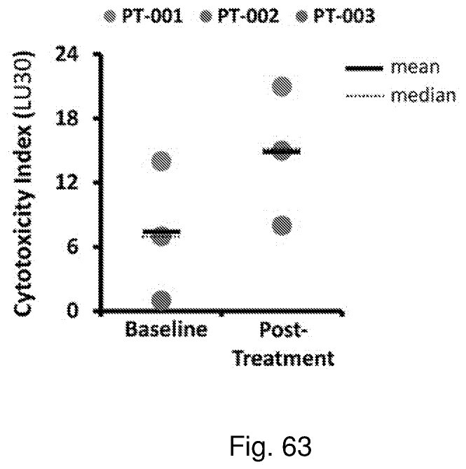

To measure how effective a patient's natural killer and cytotoxic lymphoid cells are in destroying cancerous cells, PBMC were incubated with leukemic cells. All patients tested exhibited an increase in cytotoxic function compared to baseline (FIG. 63), with natural killer and cytotoxic lymphoid function essential for proper immune surveillance against cancer and infectious disease. Increases in these natural killer cells and/or their function should also lead to improvements in treating disease states as disclosed elsewhere herein, including age-related cellular dysfunction in these patients.

Some embodiments, provide methods (e.g., through exposure to restored compositions, RNAi(s), exosomes, small molecule drugs, etc.) for increasing natural killer cell cytotoxic response. In some embodiments, natural killer cell cytotoxic response is increased by equal to or at least about: 1%, 2%, 3%, 4%, 5%, 10%, 20%, 25%, 30%, 40%, 50%, 60%, 70%, 75%, 80%, 90%, 100%, 200%, 300%, 400%, 500%, or ranges including and/or spanning the aforementioned values.

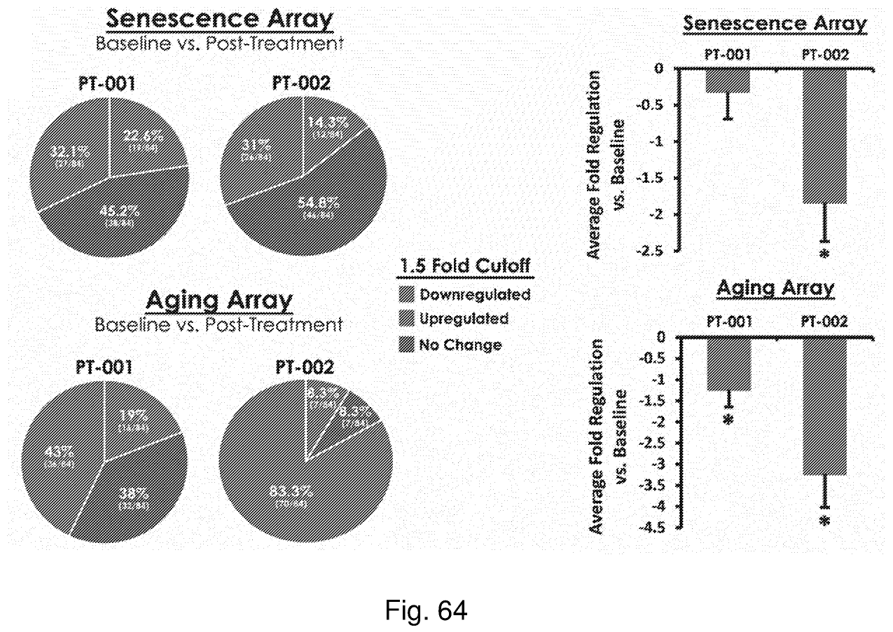

FIG. 64 shows the effect of AR-100 on the expression of aging- and senescence-related genes. Mononuclear cells isolated from peripheral blood were measured for genes linked to aging and cell senescence. Many of these genes show increased expression with age and are coincident with decreased immune function. Senescence and aging panels each contained 84 genes, for a total assessment of 168 genes measured before and after treatment. Downregulation of genes linked to senescence and aging were observed for both patients tested (FIG. 64, pie charts, left panel), with the most dramatic effect observed for aging-related genes (FIG. 64, bottom pie charts). A statistically significant decrease in gene expression was observed for both patients' aging array profiles, as well as for the senescence array profile of PT-002 (FIG. 64, right panels).

Of note, as established in FIG. 64, regarding the “Senescence Array” for PT-001 and PT-002, for example, 45.2% and 54.8% of genes have no change, while 32.1% and 31% of the senescence genes are downregulated, and 22.6% and 14.3% are upregulated, respectively. Some embodiments provide methods (e.g., through exposure to restored compositions, RNAi(s), exosomes, small molecule drugs, etc.) for down-regulating genes associated with aging or senescence by equal to or at least about: 1%, 2%, 3%, 4%, 5%, 10%, 20%, 25%, 30%, 40%, 50%, 60%, 70%, 75%, 80%, 90%, 100%, 200%, 300%, 400%, 500%, or ranges including and/or spanning the aforementioned values.

### Example 16

To evaluate whether in vitro restoration could be propagated, aged and young cells from isochronic cultures (+ iso aged +young) or aged cells from heterochronic culture (+heteroaged) were harvested at day 7 and transferred to fresh transwell cultures with naïve aged cells. As control, naïve aged cells were also placed in isochronic culture. After an additional seven days, aged cells from the 2^{nd} set of cultures were evaluated by clonogenic assay. Both of the aged cultures placed in heterochronic culture and isochronic culture with restored aged cells showed comparable restoration, while the control isochronic culture with non-restored aged cells reflected a non-restored phenotype. This demonstrates that restored aged cells can be used to restore other cells in isochronic cultures.

### Example 17: Administration of a Combination Therapy

This is a prophetic example. 200 healthy aged (>60 y/o) individuals are recruited. The study participants are split into a group of 100 experimental patients and 100 control patients. All patients are dosed subcutaneously with STEMGEN® for 5 days to stimulate the bone marrow (BM) to expand the HSC/HPC compartment and mobilize it into the peripheral blood stream. On the 5th day, mobilized peripheral blood (MPB) are collected with the Spectra Optia® Apheresis System using continuous flow centrifugal technology directly into the collection bag. Leukapheresis is performed according to the manufacturer's instructions to process 18 L of blood at a flow rate of 50 to 100 mL per min. The cells of the experimental patients are cultured and treated with RNAi(s) (SEQ ID NOs: 15, 17, and 19) along with a small molecule CaMKP inhibitor shown here:

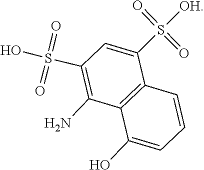 [SMILES: Nc1c(S(=O)(=O)O)cc(S(=O)(=O)O)c2cccc(O)c12]

After treatment of the cells by the combination of RNAi(s) and the small molecule inhibitor, the treated cells are analyzed. It is noted that the treated cells in the experimental group have statistically significant increased innate immune function, increased telomere length, and lower replicative stress relative to the cells from the control group. A portion of the cells are cryogenically frozen and banked. A second portion of the experimental cells are reintroduced to the experimental patients and the control cells to the control patients. After 5 years of patient tracking, the experimental patients report becoming sick less often (25% reduction) than the control group, having a lower incidence of cancer (60% reduction), having less soreness in their joints (45% reduction as measured using the VAS score), and generally report feeling better. The experimental patients also report improved cognitive performance and function (35% increase relative to control as measured using the Cognitive Function Composite Score), have measured improvements in cardiovascular output (increase in cardiac output of 42%), and muscular health (as measured by strength increases relative to controls during weight training). The results are statistically significant.

### Example 18: Administration of RNAi(s)

This is a prophetic example. 100 healthy aged (>60 y/o) individuals are recruited. The study participants are split into a group of 50 experimental patients and 50 control patients. All patients are dosed subcutaneously with Neupogen® for 5 days to stimulate the bone marrow (BM) to expand the HSC/HPC compartment and mobilize it into the peripheral blood stream. On the 5th day, mobilized peripheral blood (MPB) are collected with the Spectra Optia® Apheresis System using continuous flow centrifugal technology directly into the collection bag. Leukapheresis is performed according to the manufacturer's instructions to process 18 L of blood at a flow rate of 50 to 100 mL per min. The cells of the experimental patients are cultured and treated with RNAi(s) (SEQ ID NOs: 9-15).

After treatment of the cells by the combination of RNAi(s), the treated cells are analyzed. It is noted that the treated cells in the experimental group have statistically significantly increased stem cell clonogenicity, increased cytotoxic function, increased mitogen- and antigen-induced lymphocyte proliferation and activation relative to the cells from the control group. A portion of the experimental cells are cryogenically frozen and banked. A second portion are reintroduced to the experimental patients and the control cells to the control patients. After 4 years of patient tracking, the experimental patients report more active lifestyles, feeling younger, and generally better health. After 5 years of patient tracking, the experimental patients report becoming sick less often (35% reduction) than the control group, having a lower incidence of cancer (40% reduction), and having less soreness in their joints (65% reduction as measured using the VAS score). The experimental patients also report improved cognitive performance and function (55% increase relative to control as measured using the Cognitive Function Composite Score), have measured improvements in cardiovascular output (increase in cardiac output of 62%), and muscular health (as measured by strength increases relative to controls during weight training). The results are statistically significant.

### Example 19: Administration of RNA(i)s

This is a prophetic example. 100 healthy aged (>60 y/o) individuals are recruited. The study participants are split into a group of 50 experimental patients and 50 control patients. The experimental group receives a composition comprising RNAi(s) (SEQ ID NOs: 15-20) in liposomes intravenously one a week for two months. The control group receives a placebo. After 1 month, the cells of the groups are mobilized and collected. After treatment of the cells by the combination of RNAi(s), the cells have decreased senescent behavior, has increased innate immune function, increased telomere length, lower replicative stress relative to the patient cell, increased stem cell clonogenicity, increased cytotoxic function, increased mitogen- and antigen-induced lymphocyte proliferation and activation, decreased myeloid to lymphoid ratio, increased CD4 to CD8 T lymphocyte ratio, decreased expression of senescence-associated secretory proteins, and/or decreased expression of senescence- and aging-related genes relative to the control cells. A portion of the experimental cells are cryogenically frozen and banked. These cells can be stored for a period of years and used to autologously treat patients.

After 6 months, the cells of the patient groups are mobilized and collected. Six months after treatment of the cells by the combination of RNAi(s), the cells have decreased senescent behavior, has increased innate immune function, increased telomere length, lower replicative stress relative to the patient cell, increased stem cell clonogenicity, increased cytotoxic function, increased mitogen- and antigen-induced lymphocyte proliferation and activation, decreased myeloid to lymphoid ratio, increased CD4 to CD8 T lymphocyte ratio, decreased expression of senescence-associated secretory proteins, and/or decreased expression of senescence- and aging-related genes relative to the control cells. The patients indicate they have a feeling of better health. After 5 years of patient tracking, the experimental patients report becoming sick less often (75% reduction) than the control group, having a lower incidence of cancer (73% reduction), and having less soreness in their joints (55% reduction as measured using the VAS score). The experimental patients also report improved cognitive performance and function (75% increase relative to control as measured using the Cognitive Function Composite Score), have measured improvements in cardiovascular output (increase in cardiac output of 32%), and muscular health (as measured by strength increases relative to controls during weight training). The results are also statistically significant.

### Example 20: Administration of a Small Molecule Compound

This is a prophetic example. 100 healthy aged (>60 y/o) individuals are recruited. The study participants are split into a group of 50 experimental patients and 50 control patients. The experimental group receives a composition comprising, consisting essentially of, or consisting of the compounds

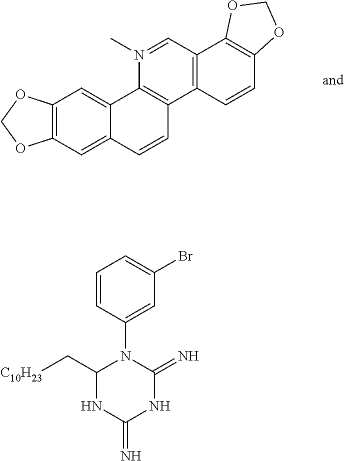

orally one a week for two months. The control group receives a placebo. After 1 month, the cells of the groups are mobilized and collected. After treatment, the cells have decreased senescent behavior, has increased innate immune function, increased telomere length, lower replicative stress relative to the patient cell, increased stem cell clonogenicity, increased cytotoxic function, increased mitogen- and antigen-induced lymphocyte proliferation and activation, decreased myeloid to lymphoid ratio, increased CD4 to CD8 T lymphocyte ratio, decreased expression of senescence-associated secretory proteins, and/or decreased expression of senescence- and aging-related genes relative to the control cells. A portion of the experimental cells are cryogenically frozen and banked using one or more methods disclosed herein. These cells can be stored for a period of years and used to autologously treat patients.

After 6 months, the cells of the groups are mobilize and collected. After treatment of the cells by the combination small molecules, the cells have decreased senescent behavior, has increased innate immune function, increased telomere length, lower replicative stress relative to the patient cell, increased stem cell clonogenicity, increased cytotoxic function, increased mitogen- and antigen-induced lymphocyte proliferation and activation, decreased myeloid to lymphoid ratio, increased CD4 to CD8 T lymphocyte ratio, decreased expression of senescence-associated secretory proteins, and/or decreased expression of senescence- and aging-related genes relative to the control cells. The patients indicate they have a feeling of better health. After 5 years of patient tracking, the experimental patients report becoming sick less often (22% reduction) than the control group, having a lower incidence of cancer (42% reduction), and having less soreness in their joints (46% reduction as measured using the VAS score). The experimental patients also report improved cognitive performance and function (39% increase relative to control as measured using the Cognitive Function Composite Score), have measured improvements in cardiovascular output (increase in cardiac output of 15%), and muscular health (as measured by strength increases relative to controls during weight training). The results also statistically significant.

Testing may include: Physical exam, vitals; Neurologic—cognitive tests (app-based); Other Biomarkers Available Through Quest—cardiovascular panels, metabolic panels, inflammatory panels (IgG, CRP, cytokine panel). Clinical data management—HIPAA compliance.

1. A method for preparing at least one target cell for use in treating a patient with cellular dysfunctional or an age-related disorder, the method comprising:
providing at least one donor cell from a donor;
providing at least one patient cell from a patient;
exposing the patient cell to the donor cell in an environment that is free of non-human animal-based factors to provide at least one target cell;
wherein the donor is younger than the patient.

2. (canceled)

3. The method of claim 1, wherein the donor cell is exposed to the subject cell in a manner that prevents the donor cell and the subject cell from becoming mixed.

4. The method of claim 1, wherein the donor cell is provided as a cryogenically frozen donor cell that is thawed prior to exposure to the subject cell.

5. The method of claim 4, wherein the patient cell is provided as a cryogenically frozen subject cell that is thawed prior to exposure to the donor cell.

6. The method of claim 4, wherein the donor cell is collected from the donor and is placed in a cryogenic medium comprising one or more of human serum albumin (HSA), dimethyl sulfoxide (DMSO), and saline prior to cryogenic freezing.

7. The method of claim 6, wherein the donor cell is placed in an equilibration medium after thawing, the equilibration medium comprising one or more of Roswell Park Memorial Institute media, Pen Strep, and Glutamine.

8. The method of claim 7, wherein the environment that is substantially-free of animal-based factors is a restoration medium comprising one or more of minimum essential medium Non-Essential Amino Acids Solution, insulin-transferrin-selenium-sodium pyruvate, and HSA.

9. The method of claim 5, wherein the patient cell is collected from the patient and is placed in a cryogenic medium comprising one or more of human serum albumin (HSA), dimethyl sulfoxide (DMSO), and saline prior to cryogenic freezing.

10. The method of claim 9, wherein the patient cell is placed in an equilibration medium after thawing, the equilibration medium comprising one or more of Roswell Park Memorial Institute media, Pen Strep, and Glutamine.

11.-15. (canceled)

16. The method of claim 1, further comprising:
exposing the patient to the at least one target cell, thereby treating the patient.

17.-24. (canceled)

25. A kit for collecting blood from a patient, the kit comprising:
liquid collection containers;
a laboratory directive;
instructions for the blood drawing from the patient or the donor; and
comprising a diagnostic testing unit, wherein the diagnostic testing kit comprises one or more of a myeloid leukemia panel, a myeloid/lymphoid ratio assay, a lymphocyte proliferative response assay, a natural killer cytotoxicity assay, a T helper cell/killer T cell ratio assay, and/or a complete blood count assay.

26. The kit of claim 25, further comprising an enclosing container configured to house other components of the kit, a shipping envelope, or a national lab directive, or any combination thereof.

27.-28. (canceled)

29. The kit of claim 25, wherein the lab directive provides instructions for blood sample processing or blood drawing instructions, or both.

30. (canceled)

31. The kit of claim 25, further comprising a patient self-evaluation form.

32. The kit of claim 31, wherein the self-evaluation form is a quality of life form.

33. The kit of claim 25, wherein the lymphocyte proliferative response assay is mitogen-based and/or antigen-based.

34. The kit of claim 25, wherein the diagnostic testing unit comprises biochemical and/or genetic biomarker assays.

35. The kit of claim 25, wherein the diagnostic testing unit comprises one or more of a senescence gene array, an aging gene array, and/or a senescence protein array.

36. The kit of claim 35, wherein the senescence gene array and/or aging gene array is configured to measure mononuclear cells in blood.


SEQUENCE LISTING (42 sequences)

SEQ ID NO: 1 - DNA, 8704 bp, Homo sapiens
agttccttaatcatttcacggtgccttcggacgctttttttccacctaaaacgtttagtttcagctcagtgatcagctaccccagctcggcgggggagcggaaggcttgaattattccgacctgtgagcggcccctggcaccaaaaaaaaaaaaaaaaaaaaaaaaaaaagaaaaaaaaaggcacaaaaaagtggaaacttttccctgtccattccatcaagtcctgaaaaatcaaaatggatttagagaaaaattatccgactcctcggaccagcaggacaggacatggaggagtgaatcagcttgggggggtttttgtgaatggacggccactcccggatgtagtccgccagaggatagtggaacttgctcatcaaggtgtcaggccctgcgacatctccaggcagcttcgggtcagccatggttgtgtcagcaaaattcttggcaggtattatgagacaggaagcatcaagcctggggtaattggaggatccaaaccaaaggtcgccacacccaaagtggtggaaaaaatcgctgaatataaacgccaaaatcccaccatgtttgcctgggagatcagggaccggctgctggcagagcgggtgtgtgacaatgacaccgtgcctagcgtcagttccatcaacaggatcatccggacaaaagtacagcagccacccaaccaaccagtcccagcttccagtcacagcatagtgtccactggctccgtgacgcaggtgtcctcggtgagcacggattcggccggctcgtcgtactccatcagcggcatcctgggcatcacgtcccccagcgccgacaccaacaagcgcaagagagacgaaggtattcaggagtctccggtgccgaacggccactcgcttccgggcagagacttcctccggaagcagatgcggggagacttgttcacacagcagcagctggaggtgctggaccgcgtgtttgagaggcagcactactcagacatcttcaccaccacagagcccatcaagcccgagcagaccacagagtattcagccatggcctcgctggctggtgggctggacgacatgaaggccaatctggccagccccacccctgctgacatcgggagcagtgtgccaggcccgcagtcctaccccattgtgacaggccgtgacttggcgagcacgaccctccccgggtaccctccacacgtcccccccgctggacagggcagctactcagcaccgacgctgacagggatggtgcctgggagtgagttttccgggagtccctacagccaccctcagtattcctcgtacaacgactcctggaggttccccaacccggggctgcttggctccccctactattatagcgctgccgcccgaggagccgccccacctgcagccgccactgcctatgaccgtcactgacccttggagccaggcgggcaccaaacactgatggcacctattgagggtgacagccacccagccctcctgaagatagccagagagcccatgagaccgtcccccagcatcccccacttgcctgaagctcccctcttcctctcttcctccagggactctggggccctttggtggggccgttggacttctggatgcttgtctatttctaaaagccaatctatgagcttctcccgatggccactgggtctctgcaaaccaatagactgtcctgcaaataaccgcagccccagcccagcctgcctgtcctccagctgtctgactatccatccatcataaccaccccagcctgggaaggagagcttgcttttgttgcttcagcagcacccatgtaaataccttcttgcttttctgtgggcctgaaggtccgactgagaagactgctccacccatgatgcatctcgcactcttggtgcatcaccggacatcttagacctatggcagagcatcctctctgccctgggtgaccctggcaggtgcgctcagagctgtcctcaagatggaggatgctgcccttgggccccagcctcctgctcatccctccttctttagtatctttacgaggagtctcactgggctggttgtgctgcaggctccccctgaggcccctctccaagaggagcacactttggggagatgtcctggtttcctgcctccatttctctgggaccgatgcagtatcagcagctcttttccagatcaaagaactcaaagaaaactgtctgggagattcctcagctacttttccgaagcagaatgtcatccgaggtattgattacattgtggactttgaatgtgagggctggatgggacgcaggagatcatctgatcccagccaaggaggggcctgaggctctccctactccctcagcccctggaacggtgttttctgaggcatgcccaggttcaggtcacttcggacacctgccatggacacttcacccaccctccaggaccccagcaagtggattctgggcaagcctgttccggtgatgtagacaataattaacacagaggactttcccccacacccagatcacaaacagcctacagccagaacttctgagcatcctctcggggcagaccctccccgtcctcgtggagcttagcaggcagctgggcatggaggtgctggggctggggcagatgcctaatttcgcacaatgcatgcccacctgttgatctaaggggccgcgatggtcagggccacggccaagggccacgggaacttggagagggagcttggagaactcactgtgggctagggtggtcagaggaagccagcagggaagatctgggggacagaggaaggcctcctgagggaggggcaggagagcagtgaggagctgctgtgtgacctgggagtgattttgacatgggggtgccaggtgccatcatctctttacctggggccttaattccttgcatagtctctcttgtcaagtcagaacagccaggtagagcccttgtccaaacctgggctgaatgacagtgatgagagggggcttggccttcttaggtgacaatgtcccccatatctgtatgtcaccaggatggcagagagccagggcagagagagactggacttgggatcagcaggccaggcaggtcttgtcctggtcctggccacatgtctttgctgtgggacctcagacaaaaccctgcacctctttgagccttggctgccttggtgcagcagggtcatctgtagggccaccccacagctctttccttcccctcctctctccagggagccggggctgtgagaggatcatctggggcaggccctccacttccaagcaagcagatgggggtgggcacctgaggcccaataatatttggaccaagtgggaaacaagaacactcggaggggcgggaatcagaagagcctggaaaaagacctagcccaacttcccttgtgggaaactgaggcccagcttggggaaggccaggaccatgcagggagaaaaagcagacttcctctggccaccgctaagtattttgttccctaagtcccccacagggtggtggaacagaagagaaaactaaggctcagcaaggtggagtcctcaagcagtgactggtgggggtggggctgggactcgggctcctgacccccaacccatggtgttccctgtcaccctggtctatccacatctcctattcctgaggagagttgacagtaagagcagtgagagatggttctgggccccatgccctagacaatcagtctgtaagaactgccaaggaagcctggtcacccaggccagggatggagcccagcgagctcacagcaggcacatgcacccccgcccccacccagaacctgcggggcaaagaagggaggtagttggggccagagtccacctccaggagccagggtgagctggctgcagcttccacctgtcaggtaaggtaggagaaggtatgttacctggcatctctctccctgcctccctccatttagcaggaagtggtggggtcaggggtcttcagcacagacttcttgagcctctgccccctgtcacccttcttttggaatgatgtgtacccacatctttggatcctgccctccttgtggttccaggctgttgggaggaggtcagcctcccctgaaccagctgcctgaggcatgcagcatccttcctcggcaaagcccacctggctcacagcggccccctctccccatcctgttcctcttcttgccctgtaatgagctccccccatacctttcctccctcacctgaggctttgctggtcctcagattggttttgtatttgtgagacaaccacttgactcctgggctgccaggcggaagcacaagcgcacatggatgcacacggatgttctcacacacacacgcccgctctcaccaattcacagcacctcgtggtccagcggagctgcctgggagcttggtgaggatggctccaaaggacacaagccgttgagtagatgccagagaattctgaatggaaaacacaagtccggggcctcaccagcatcgtggcagaaggcctggggcatttctccatgggccttcttccctgtgttcgagctctgacttttggaaaaggacattgtggattttatgaaaatttctcataccatcagtctagctccaacctagaaaaattggatgatatatcaaacccaacatccctttcccaaggcaccttagttaaggctagcccttccataactgacattgtacggtgctttgcaatcctcaaccactctgtggagcaaagagcatgatgcctactttatagacggggaaattgacatttggggctctcacggcaacatagaggcgaagtgaattctcagatctccaggccccattctctctccaccgagttgtgctcctgtcatgggagtgtatggcttaaagacactcccccacccccattccctagaaatcccccagacccacaatcaggcaagaaagaacagggacccagaggctggcctgacctaggggcctgcaggttggcggctttgtttcctaagaacattgaaacctgcggagtctttgaccaaatccacaacagtgcttctgaggtttcatccagactctttcccagctgtccctgaggttcagagggtatcagagtcaattcaaggccatgccataatccctgaccaggcctggacatgggtccagccctgactccaggggtccaggtgccaaggtcatgtgctgtcccccacttccctttctttgccttcgcactttggaacaggctccaggcctggctgtgacagtatgcaggagtgcccaagccagggccaccagggtgtccacagcccctggaaacacagatcacaatctcaagtcccctgaatgaactgcttcctgggtgaacggggtggtgtagccttgccactcgggcagcgcaccagacagtacggtgcagcagtgcccgagatgcccagaagtgtgcctgcccccctgagtggcattcaagtatgaaaacttgtaaaattttctgtcggcctaaagaaaaatgtccatgggccaaacttgacccactgggcaccagcctgtgagccccaatgcctgtcaattgccccctttctcattcacctcctgtcctttgttgcagatttggaggagatgggagcccagagaggtgagagatgtgcttcagggtgtccagcaaggcaggggtagaaacgggcacgcccagagcctgccactctcccaggcctcatcttgggctcttgcaagtcttgcgctttgaagatggagttgggtggaaggtcagaggcgctggggacgggattggggagctgctgggttttcaggggaggtcagtgctgcttgggcacctttcacatgtgcagggaagagactcagatgtggccacagggcactgaggggtgaaatccatttaccagaagtcacgctccagaacgactgcccagccttggcagccagtggctccagccaccccctcctcatgacattcagccagaattgagctccaagccagggaagcaaatcttaaaaaccaaccaagcaccctgacacagccctagaaacacgatgagtctgaaatacagcttcccaggaggggagtctaagatacagcttcccgggagggtggaaaccaactcctgccccacggccaggccaggcccaggcaggcatgggtggatcccacagggctctgagctagaccggctccacggtggccccactacgaggccagttctgcagtcttggccttgtcacccatcgagaggctgctctgatggttcccagccacacaccagctctcctggggaaactatttctttcgttcttttggcctcggagaggtccgaggcaagtacatttcttaaaaggtaataaaatgcattattggaaagttggacagtcaggccacgactcctagcccacggcgtgccccacctcccagcagccccttcagccccttgcccctgttgccccaaacctcagggttccctcttgcatattcatgggggaaccacagtgctgatgtgtacttccccactgtcagctcggctgcacctcgtgtggcgccaggtcccaagggcctctgcagaggccaggctgtgagccccttgcctgcctgctcccctgatgaggcaacagcttctctgaaatgagctgccggccaggagcaggcaggcaccagcctgtctttccttttctggtaattcctcagcactgaggctccgttcctgggcaccccaggattgaagggaacctcagaatcatgtcactgccattctagagtttcaatccaaggggtcccctttagctcatctccaagatgggtaaacgtagccaccattcagaaagcccagaaattcttgttcccacatcttagacccctgagcaacacaaggagaaaatgcagctgcttacctattaatatctactgagggacaatcagcaaagcctcaaaggagtcgtctcaggtagggtacttggcctgtggcaggagagacagaggcacaaacccacccacccatcagcttccggtggctgatcggggcccagggagggaagcaggtgaaacagcagggtgggggtgacttggcagtcgtgcatctcctccccgactctgaggcctggcaggaggaggccacaccagcctcacctgccctgacccccgccccccaccctgtgaccctgtggctatggccgctggtcgcccttgtccccaaaatcaccatgcctgtggccacgtccctcatcttctccaaaagcatcattaagaacaagtgattttggatgatggatttttgatttacaaacggcgcatttcccttggagtggaacagaaaggaaaccatttaatggcgcccttcttttcaagcatgaatacattttaatgaaactattttattgtatttgaggaaatggagagttgaacattccaaccaatcaatagccaattaattgctataaagctaaaaagaaaataaataaatcctgagtctattttaaacactgcaaaaagttcagagcctcagaatctggccttcccctccataaggtgcacgagcatgtaaacacacacacacacacacacacacacacacacacactcactcactcccctacacctcagacatacttgaaactcagaaacagcactgagtctccccatgccaattcttgcctgctgtcttcgacttgggtcagagaaggtgagcagacccggcagcagcctgtcccggggctcaggaagaggcaggcccatcccctggccccaagcacccagcacaacagagggtggcgggcagtgagggcctggcgttgcctgggccccacttctcagccccagctgctgggcctccaaggttgggctgaggatggagttttggctctgggtttgccctgactcctgctggaagacgctgccctggtttttcaccctctagtggccttggacattgagtatttgtagaaatgcagattacattgcaaatggaaacctttgccaggaagacacatgcattttgcttttaattctttgagacatttgattttgtcttagggactgacctttcagcatcaaagaaatacatatctactgtatccgccaaagtttgtgatgcctgcatagacgcttacttgtaaaaaaaaaaaaatacaaaaaaatacaaaaaaaccaacaacaaaaaccacaattgaattgcctttgaaagtgggagatgatctgtctccaacggattgaaaaaaaaaaatgcttcttaaaaaatgtgtatgttttgtattctttttttctagtagaaaataactgacttgaaatattggtggtttttttcttagtgacgtgtgttgcttttgtgtgtaataatatttgaatgtaattacagcagtgccaatttgccaaagatgttggacatatttttcttttttggggaggagggcagggctaggggtgggacttgggagaaaacaggggtggggttttggtttaattttttttttacttttttttccttgtcaaacctgaaatttgtggcttccttttaagttaaatggttgactgcaacacctttattttagattagttggagaaacatgcaataagattggcgtagtttcaatatctgtgtgtcttttcatgagtggctgttacttgtgaagaattgattttatgtaacctttatgtgagataattatttgtaaatatttgccataattttattggttcctaaaataaaagtaattttttaagttcagaaaaa

SEQ ID NO: 2 - DNA, 1212 bp, Equus caballus
ctgtccattccatcaagtcctgaaaaatcaaaatggatttagagaaaacttacccgactcctcggaccggcaggacaggacatggaggagtgaatcagcttgggggggtttttgtgaacggacggccactcccggatgtagtccgccagaggatagtggaacttgctcatcaaggtgtcaggccctgcgacatctccaggcagcttcgagtcagccatggttgtgtcagcaaaatccttggcaggtattatgagacggggagcatcaagcctggggtaattggaggatccaaaccaaaggtcgccacacccaaagtggtggaaaaaatcgctgagtataaacgccaaaatcccaccatgtttgcctgggagatcagggaccggctgctagcagaacgcgtgtgtgacaatgacaccgtgcccagcgtcagttccatcaacaggatcatccggacaaaggtgcagcagccgcccaaccagccggtgccggcttccagccacagcatagtgtccacgggctcagtgacgcaggtgtcgtcggtgagcacggactcagccggctcctcgtactccatcagcggcatcctgggcatcacgtcccccagtgctgacaccaacaagcgcaagagagatgaaggtgttccggagtccccggtgcccaacggccactccctgccgggccgagacttcctccggaagcagatgcgaggagacctgttcacgcagcagcagctggaggtgctggaccgcgtgttcgagaggcagcactacgcggacatcttcaccaccacggagcccatcaagcccgagcagaccactgagtattcagccatggcctcgctggctggagggctggacgacatgaaggccaacctgaccagccccaccccagctgacatcgggagcagcgtgccagggccgcagtcctaccccattgtgacaggccgcgacttggcgagcacgactctccccgggtaccctccgcacgtccccccggccggacagggcagctactcagcgccgacgctgacagggatggtgcctgggagtgagttttccgggagcccctacagccaccctcagtacccctcgtacaacgactcctggaggttccccaacccggggctgctcggctccccgtactattacagtgccgctgcccggggggctgccccgcctgcagctgccactgcctatgaccgtcactgaccct

SEQ ID NO: 3 - DNA, 7765 bp, Canis lupus
gaaagaaaaaaaaagaaaggaagaaagaaaagaaaaaagaaaaaagaaagaaagaaaaagaagcacacaaaaaaagtggaaactttttccctcgtccacttcaagttctgaaaatcaaaatggatttagagaaaaattacccgactcctcggagcggcaggacaggacatggaggagtgaatcagcttgggggggtttttgtgaatggacggccacttccggatgtagtccgccaaaggatagtggaacttgctcatcaaggtgtcaggccctgcgacatctcaaggcagcttcgagtcagccatggttgtgtcagcaaaattcttggcaggtattatgagacggggagcatcaagcctggggtaattggaggatccaaaccaaaggtcgccacgcccaaagtggtggaaaaaatcgctgagtataaacgccaaaatcccaccatgtttgcctgggagatcagggaccggctgctggcggaacgtgtgtgtgacaatgacacggtgcccagcgtcagttccatcaacaggatcatccggacaaaagtacagcaaccacccaaccagccggtcccagcttccagtcacagtatagtgtccacgggctccgtgacgcaggtgtcgtcggtgagcacggactcggccggctcctcgtactccatcagcggcatcctgggcatcacctcccccagcgccgacaccaacaagcgcaagagagatgaaggtattcaggaatctccagtgccaaacggtcactccctgccaggcagagatttcctccggaagcagatgcggggagacctgttcacgcagcagcagctggaggtgctggaccgcgtgtttgagaggcagcactactcggacatcttcaccaccacagagcccatcaagcccgagcagaccaccgagtattcagcgatggcttcgctggctggagggctggatgacatgaaggccaacctgaccagtcccacccccgccgacattgggagcagcgtgcctgggccgcagtcctatcccattgtgacaggccgtgacttggcgagcacgaccctccccgggtaccctccgcacgtcccccccgccggacagggcagctactcagcaccgacgctgacagggatggtgcctgggagtgagttctccgggagcccctacagccaccctcagtatccctcgtacaacgactcctggaggttccccaacccggggctgcttggctccccgtactattacagcgccgcagcccgaggagctgccccgccagcagccgccactgcctacgaccggcactgaccctcggagccaggcgggcgccaagcacttatcacacatatcactgagggcggtagcctctcggcccctccgaagatggccagaggggcccagcgagaccatcctccagcaatccccactcgcctgaaactccctcccaaccttttcttgccaaggactctggggctctatggtaaggctgttagacttctagacacgcgtgtgtttctaagagtcaatcagcgagctgctcccaacagcaactgggtctctgcaaagcaacggactattctgcagacaactgtagccccagcctagcctgccagtccccagctgtctgaccatccacctgtcttcctgccccaggcctgggaaggagagcttgcttttgtcacttcaacagcacccatgtaaataccttcttgcttttttgtgggcctgagggtccaactgaggcagaccgcctccccattatgcatccagtactcccgatgtgtcacaggacatcttagacctgtgtgcagagcattccctctgtcctggctgccccccacaggtgggcttgtagttgtccttgagactaaggatgcccacctcagggcccagcctcctgctcattgctacttcttcaacgtctggactaggagtcttgttggactggttatgcttcacgcttcccccctgagacccttcctcaggagaaacacactggagagttgccctggcttcctgactcccatttttcctgggcccagtgcagtgtgagcagctcttcttccatagcaaaggactcagagaacaaagaactgtctgggagattcctcagctacttttctaaagtaagatgctgtccaaggtgctgaccgcattgtggactcagtgctgcggctgggcaggacccagaagaccatagtccaaggcgaggactcccaagcccctggaaccactgaactgaactgggatgtgtttctgaggtgttcccaggctcatatcacattggacacccaccatggacatttcttccaccctccaggatgctagagagtggattctgggcaagcctgtccctgccatgtagacaataattcacaaaaaggaagttcttgcaccaaggaccacaaacccctacagcagtcagccagaacttcctgatcagcctctcccaattcctgtggagcccagtgtgcagttgggactagaggtgctggggtgggagcagatgcctaactgcacagtgggtatatacctattgatgcagggggttgtgatggtcagcaccatggccaagggcccctggagcctggagtgggagtttggagaactcactgtaggctggagtggtcagaaaaaactacctgggagaatgtgggggacagaggaaggccttctgggagagtgaggaactggtgtgtgaactaggagtgattttgaaataggggttccaggggccactatctctttgcctgggacctcaattccttcaataatctctcttgccagctccaagcagccaggtggaggtctgtccaaatctgggctgaattacagtgatgacaatggactttgccatctcatgtgacaggatacccaatgtctacatgtcaccaagttggcaaagagctggggcagagcagagcctgcacttgaggctattggccctgagaggtcttatcctggccacaccatttccttgctgtaggacctcagacaagaccctcacctctctgagtctccactgctttggtacagcagggtcacctgcagggccatcctatagttctctcctcagcttcctctttccccagatgctggggctgtgactgggtcatctggggcaagtcctctacttataagcaagcgtaagagttgggcatctgagtcctgctaatatttggaccaaatgggaaataagaacacccaaagagatgagaatcagccttgaggaagatctagcccagctcccctcaggggaaactgaggcccagtttggggaagcccagtatgatgcagggaaatagaacagatatcctctggccactatcaagtgttttctccccaacatcctacaaggtggtggaagagaagaggaaacaagcttaccaaggtggaggccacaggcagtgactggcagaggtagggctgggattcaaccccccaacccctggcacaaggtgatcatccttccatccctggggagggaagttgacagtgagggcagtgggagatcgttctgggcccactgccacagatagagtgttctgggctgttagaactgccaagaaagcccaggcacccagggcagaggtggagccagtgggctcaccataggcacacccacctccttggaacctgctgggcaaagaagagaggcagctgggggcagagcccaccttcaggaacaggatgaaccagctccagcctccacctgccaggcaaggtaggagaaggaatgtcatctggcctctcctttcctccttccctccctccttcccttccacaggaaatggttggctcaggagtctgaagcacaggctcttggcctcttgcacccacatctttggtcttcatccctccttgtggtctcaggatattgggaggtcagcctttcctgaaccaactgcctaaagcacacactgtccttcctcatcaaagcccagccggctccattctgttccttctcttgccctgtgatgaggtccccccatacctctcctctctcccccgaggctctgctggtcttcagattgcatttgtatttattagacaaccacttgattcctgggctgccaggaagaagcacaagagcacatggatgtacacgcatgttctcacacatgtacctgctctctccagctcagggtacacacactgcccagtggaacgcctgggagcttggagagaaaggttccaaaggacacaagccgttgagtaaatgccagagaattctgaatggaaaacacaaggccagagctctcacgaacattgtggctggaggcctggggcactcctccatgggctttcagccctatgttccagctctgaattttggaaaggaccttgtggattttatgaaaaagcttcatatcattagtctagctccagccttgaaaaatctgatgacatatcaaatccaatctcccttttccaaggcgccttaggtagggttagccctttcataactcacatttgtatggtgctttgcaatccttgaccattccctggggcaaagaccacaatgcctattctacagatggtaaaactgagatttggggtttgcatggcaagagagaggtaaagtgaattctcagatctctgggccccattctctctccacagaattgtgctcttgtcatgagagcatgtggcttagacaaatgcttcaatcccctagaaaccccctagacccacaaggaggaaaaaaagaacagggacccagaggctgccctgaccaaatagcctggggcttggtggcattgccagtctaccttgattctcattaacaccaaacctgcaaagtctttgcccagatgcattcatatcagtgcctctgaggttttatccacacttcttccctgctgggtcctgagcctccaaggggatcagaccacaccattagccctggcagagcctagacacgggcccgcctccaactcccagggtctaagattccagggttatgagatgtcccctcactggggttcagacatcagccctgtttgtgacaacacgtaggagtcccccagcagggccctcagagcagccacaggcctgggaaacacagatcgcaatctcaagggccctgaactgctgcttcccaggttcagacggagtaatcttgtcgctcaggccgggccagaggcaaccagagggcttgccccaaccaacagcattcaaatgtgaaagcttttaaaatgctcttccagccaaaaacctacatttgtgggccagatttgacccaccaaccactagcacataactcccaacttttgtccagtgctccctttctccttcacttgctcttcattgaagatctgggggagatgggaacccagagagggatgggcttcaggttttaggcagtcactgtcagggacaagcatccccagtccttaccccctccctcctcaccccgggctcttgcagaccatttgggctttgaagtggatgggatagaacagtttgggggtggtgggggtggagggactattggcttcgagaggaggtcagtgccttgccgctggggtctttcatatgtgtgggaaagtgccccagatgtggccacagggcactgaaggttgaaatccctttatgagaagtcgtatcccagagcaattgccctgcctttggcagcctctggcaccagctacccctcttctggatgacatttcagctagaatttagctccaagccatggggagcaaatctcaaaaataaaccaagcactcctgacaaatacttagaaacaagatatgtctgagacacagctgcccggagggtagagacaaatccaggcccctggagaagccaggtccaggctgaagtgggtggatccctcagcactctgggcaacaccagctcccgggtggccccagctaagaagctggctttgcaaccatggccttgtcacccatctaggggcctgcctctgatggtttccaggcacgcacgtcagtgctcccggagaatttattcctttagttcttttggcctcggagaggtccaaggcaagtgcatttcttaaaaggtaacaaaacattatcggaaagttggacagtcaggccatgactcctagccggcagtctgccccacctcccagcagccccttcagcccctggccctctgacgaccccttacctcaggattccctcttgcatattcatggcggagccacagtgttgatccgaacttccccaatgtcagctcgtggctgcgcctccagagtggcggccaggtccccaggggctgctgcggagggctggaagtgagtccctggcctgcttctcgacgtgggcggcttgctaggagcagccaggcaccagactttctttcattcactgtgatttctcgacactggggttccgttcccggacccccccacccaccacccaccccaggatccaaggagacctcaggatcttgtccctgccgatctaagctgcagtgcatgtatttccaggggtccatttagtccatggctgagttgggggtaaatgtaaccaccacgcagtaagtctagatagtctccttccaacatcttagaccccaaagcagcacaaggagaaaacgcagctgtttatctatttatggaggaaatcttaatgaattctaaaatggagttggtctcaggtagcgtacttggcctgtgagtgggacaaggcgggtaggaatcccacctgatttcagctctagacagctctgagcatggctggggaagggagcatgtgaaacagcagggggcggggcagatcggagttagtgcccccttgcctgatctaaggcctgcctggaggaggtcaagctgagcacccgccccaagatcgggcccacccagtcctgcaactatggccaccaggccaccctcatccccaaaatcaccacgcataaggccaagtccctcatcatctctaaaggcaccattaagaacaagtgattttggatgatggatttttgatttacaaacggtgcatttcccttggagtggaacagaaaggaaaccacttaatggctcccttattttcaagcatgaatacattttaatgaaactattttattgtatttgaggaaatggagagttgaacattccaaccaatcgatagccaattaattgctataaagctaaaaataaataagtcttgagtctttttgaaatgcccctgcagaactccgagcctccggatcaggccttgccctccatatgtgcgcctgtgtgtacatgcattcccacatgcgcacccctgcacctcagacatacttgaaactcagagatcgtgctgagtctcctcatgccaattcttgcctgctctcctcgcctctggtcagagatggtgagcagacctggcagcagcccatgatggggctcaggaagaggcagggccatcccttggcccggcaccaccagctcagtgtgggggatactgggcagtgagggccccctcgttggcccccactgctgagcctccaagtctgggccgagggtggaaatttgacttgggttttgccctgactcttgctggaagatgctgtcctggttcttcaccctctagtggcctttggacattgagtatttatagaaataaatgcagattacattgcaagtggaactctttgccaggaagacacatggatttagcttctcattctttgagacatttgactttgtcttagggactgaccttccagcatcaaagaaatacatatctactgtatccgccaaagtttgtgatgcctgcatagacgcttacttgtaaaaaaaaaaaaaaaaaaaaaaaaaa

SEQ ID NO: 4 - DNA, 2426 bp, Felis catus
gggaaaaaaggaaaagaaaagaaagaaaaaagaaaaaagaaagaaaaagaagcacacacaaaaagtggaaactttttctccctgtccacttcatcaagttctggaaaatcaaaatggatttagagaaaaattacccgactcctcggaccggcaggacaggacatggaggagtgaatcagcttgggggggtttttgtgaatggacggccactcccggatgtagtccgccaaaggatagtggaacttgctcatcaaggtgtcaggccctgcgacatctcaaggcagcttcgagtcagccatggttgtgtcagcaaaattcttggcaggtattatgagacggggagcatcaagcctggtgtaattggaggatccaaaccaaaggtcgccacgcccaaagtggtggaaaaaattgctgagtataaacgccaaaatcccaccatgtttgcctgggagatcagggaccggctgctggcggaacgggtgtgtgacaatgacacggtgcccagcgtcagttccatcaacaggatcatccggactaaagtacaacaacccccgaaccagccggtcccagcttccagtcacagcatagtgtccacgggctccgtgacgcaggtgtcgtcggtgagcacggactcggccggctcctcgtactccatcagcggtatcctgggcatcacgtctcccagcgcagacaccaacaagcgcaagagagatgaaggtattcaggagtctccggtgccgaacggccactcactgccaggcagagacttcctccggaagcagatgcggggagaactgttcacgcagcagcagctggaggtgctggaccgcgtgtttgagaggcagcactactcggacatcttcaccaccacagagcccatcaagcccgagcagaccactgagtactcagctatggcctcgctggctggagggctggatgacatgaaggccaacctgaccagtcccactcccaccgacattgggagcagcgtgcccgggccacagtcctaccccattgtgacaggccgtgacttggcgagcacgaccctccccgggtaccctccgcacgtcccccccgccggacaaggcagctactcagcaccgacgctgacagggatggtgcctgggagtgagttttccgggagcccctacagccaccctcagtatccctcatacaacgactcctggaggttccccaacccggggctgctcggctccccatactattacagcgccgctgcccgaggggctgccccacctgcagccgccactgcctatgaccgtcactgaccctcggagccaggcaggcgccaagcacttataacacatatcactgagggcgatagcctctcagcccctctgaagatggccagaggggcccaacgagaccatcccccagcaaccctcactcacctgaaactccctcccaaccttttcctgccagggactctggggttccatggtaaggctgttggacttgtagacatgcgtccatttctaagagtcaatcagtgagcttctcccaacagcagctgggtctctgcaaaccaacggactattcttcagacaactatagccccagcctagcctgccagtccccagctgtctgaccatccacctgtcttcctgccccaggcctgggaaggagagcttgcttttgtcgcttcaacagcacccatgtaaataccttcttgcttttttgtgggcctgagggtccagctgagcggacagcccacccattatgcatccactactcccgatgtgtcacaggaaatcttagacctgtgtgcagagtatcccctctgttctggctgccccccaagggtgggcttggagttttccttgagactaaggatgcccacctcagggcccagcctcctgctcattgctacttctttaacgtctggactaggagtctcattgggctagttatgcttcaagcttcccccctgagaaccctcctcaggagaaacacactctggagagatgccctggcttcctgacttcatttccctgggcccaatgcagtgtgagcagctcttttccatatcaaaggactcaaagagaactgtctgggagagtcttcagctactactctaaagtaagatgctgtccaaggtgctgactgcattgcggactttgaatgctagggctggacaggacccaggagaccatagtcctaggcgggaaccccaagcccctggaaccactgaactgaactgggacgtgttttctgaggaattcccaggttcatatcataccggacacccaccgtggacatttctcccaccctccaggatgccagagagtggattctgggcaagcctgtccctgccatgtagacaatgattaacaaaaaggactttcctgcactgaggaccacaaacacctacagaagtcagccagaacttcctgatcaacccctcc

SEQ ID NO: 5 - DNA, 5149 bp, Homo sapiens
agcagcttgcgggacacggagccgcgaggagacagctgaggcccgcggagaccagggggtgaagcctggagaccctcttgccctggcctagctgcaggcccccgggatgctttgggcatgtcctctggagccccacagaagagcagcccaatggccagtggagctgaggagaccccaggcttcctggacacgctcctgcaagacttcccagccctgctgaacccagaggaccctctgccatggaaggccccagggacggtgctcagccaggaggaggtggagggcgagctggctgagctggccatgggctttctgggcagcaggaaggccccgccaccacttgctgctgctctggcccacgaagcagtttcacagctgctacagacagacctttccgaattcaggaagttgcccagggaggaagaagaagaggaggaggacgatgacgaggaggaaaaggcccctgtgaccttgctggatgcccaaagcctggcacagagtttctttaaccgcctttgggaagtcgccggccagtggcagaagcaggtgccattggctgcccgggcctcacagcggcagtggctggtctccatccacgccatccggaacactcgccgcaagatggaggaccggcacgtgtccctcccttccttcaaccagctcttcggcttgtctgaccctgtgaaccgcgcctactttgctgtgtttgatggtcacggaggcgtggatgctgcgaggtacgccgctgtccacgtgcacaccaacgctgcccgccagccagagctgcccacagaccctgagggagccctcagagaagccttccggcgcaccgaccagatgtttctcaggaaagccaagcgagagcggctgcagagcggcaccacaggtgtgtgtgcgctcattgcaggagcgaccctgcacgtcgcctggctcggggattcccaggtcattttggtacagcagggacaggtggtgaagctgatggagccacacagaccagaacggcaggatgagaaggcgcgcattgaagcattgggtggctttgtgtctcacatggactgctggagagtcaacgggaccctggccgtctccagagccatcggggatgtcttccagaagccctacgtgtctggggaggccgatgcagcttcccgggcgctgacgggctccgaggactacctgctgcttgcctgtgatggcttctttgacgtcgtaccccaccaggaagttgttggcctggtccagagccacctgaccaggcagcagggcagcgggctccgtgtcgccgaggagctggtggctgcggcccgggagcggggctcccacgacaacatcacggtcatggtggtcttcctcagggacccccaagagctgctggagggcgggaaccagggagaaggggacccccaggcagaagggaggaggcaggacttgccctccagccttccagaacctgagacccaggctccaccaagaagctaggtggtttccaggcccctgccctccccttcctcccatccttgtccttctctccctcagaagcctcaggacccaacaggtggcaggcagtggacagggtgcccgccccacagtgctttccccagcaccccagagccagtcgggacaccccccgcagcccgtcctggtggctgtggaactgcactgggtggcgggcagatggtggaaggcagcttaggagacctcaccaaagagaagatggaccggctcttgctcccagctcctattaggcccggggtgggaccagaggtcataggtgcccaacggcagccaaaccaaagacactggtgtgcatggggcagcatggttgtgcacgtgggaccctggggcggacccaggagccaaactcttgaagcaccccctgggtcaggcccagcagcggagtggccagccccagtttcccattgctcctctctgcggccagggccaggtgggttcatatttacagatatgcccagccagtcctggtcggccacaccagtgtcccaaagaggagagcgcagcagagccaggggtctgttctgtagcagccacccccctgcccccactccagggcagccatgatgtgcttgggcccaccagggccttccgggctgctctcttccctgagcccggaaccggcgacgcacatgtgtcttttgttggtgtgtttgtttttttccagggaggtctaattccgaagcagtattccaggttttctctttgttttatcagtgccaagatgacctgttgtgtcatataatttaagcagagcttagcatttattttattctttagaaaacttaagtatttacttttttaaagctatttttcaaggaacctttttttgcagtattattgaatttattttctaaatcaggattgaaacaggaacttttccaggtggtgttaataagccattcaagtgccttacacagctttgaagaaactaggactgcagtgggctcggataggcccattgaggtttttagaaaagcaggatttgttttgttagggaggcatgattttggtgagatctttctggaagagttttccgcctctttgtgatgctgaacacccccaaggttctcccctccccccgctgcccaggtgactggcaggagctgcgactgccacgtagtggtgcctgggcccgacagcggggctctgggcatcccgggtgaccttggcccatctgcctgcattcccacccccttgggcctggctggatcccaggcagagggaccttgctgctgtgtgattggaacattcccaaatatcttgtgaatttgtaatcaaattggtctcattgggaaagactcttaattaagaggctcaggcaagcacagaggcagcccgtgggtctctgtctcagtctggaggcagcagggatgctgctgggagtccatggcacaggccacagcccctcaccttgccgcggtggctggcagcacgcctgccttgctctgccccatgccctgaacaggcatgagagctccacgtcccctagtgcaccctgagagggggctcacaagtgaccgatcctgggtgcctcagggagctcactgagggcgtgcaaagttgaaagtggcaaggctgggggagggtgtcgggtagagggaagagggcagggggctaggggaggactcagaggccatctgcagggccaagccacaggaagggctgagctggaggtgggcagggctgctccaggcaggtcagagcagtgcagggggaggagaggagaaagggaggaagctgggctgtgtggtccccatgaaggcattcagagtccacctgcagacagcgagagccccaggaaggtttgcacagctgtgccccaagcaccttggcctcctctcagctcgccgaggaggcacgctagagccgccttcccggtgggagccctctgtcccacagggagcggggagccagctttgctggggccctacctgcatgcccagccttacccctcattctcacagcacagatgaggttgagaccatgcagtcaatgcattgcttaaggtctcttatttacaaaaaaaaaccttaaacatagtcgctgtcattcagacattcagagaatggttggccacaaacaatgaccaagtattgcttggcttaacttgaaggcctgctgtctccttctgggggtcagggacgcagctccaccctcaccactagcccaccctgcccgtgggcataaccttgacgaagagagagaatgattggcatctgcttttctcttttctttgctaataattctgttcctggctgccgagagtgaagtttcaccatgtggaggtttggctcctatcacctggtggtctgattcataccctagcctgaggctccactggaagatctcgcagcctcagtgtatgggaaaccctttccccaggcttgtcccagcactgccgctccccacccctgagccaggaccccagaggatggccatgccccgtgcctggcagaggtctggtgccagcactgggagctgctccgcccttgccttggggccgagggagccctcgtccacccctgcacagcagctgggcacagaggagcgctcttccatcttgaccaggactgcaccaagaagcaccaggtgtcttcagcctccaacctccggggcgaccttctcttccagccacagtcccatgagggcccctagccagggacactggtctgtaaattgtaatcctttctccagcccagctctccacttgttccttgtgtgagctgagcaggcagtgcacctctgagtgtcccttttgtaaggcccaggggttgcactgagtctgcagaggccgcgacctcctagaacgctgtgggtgcaggtgagccggcgtgtcctggggagatgctgccagcacacaggggccctcctgctgccagcaggttggggtggttaagtcttattagtgtctattcttaaaattaagtgggctggagaagaatggagctccacatgccagcaccgtatatggaatacaaaagctggggaagcagggcctgccttacaggtgtggctgactctgagcccaggcctgcaggggtggagggcagtccctcagaatcccagaggcagtcccagcctcagaacccaggataggaaatgggtgtgtttagtggggaaagggacggggtgcagacggcagggccagtatggggccccctccctctcctctcctctcctatggtgagcccagcgtgggcaccgggccgtctcagccgtgttcccagggctgggaggacagctctggcccttcttaggcctagcctcgtcccaagctaaatgtaagccagttgggctgtgttaaaggaagcagtgtttttggttcgattctgcctctgtagctcaaggggggcagcccccagagtcctgtgcattctgccaaggctccatagctttgccaaatgcacggagctctgccattccggtgcagtgcaggccttgcgaagggtttatctgcgttcgtctcggtgggcttctcctgcatgggagttgtgttcctgtgcaagggggagctttgctccaggacaggatgactgtcttccctattcttagggacaagtcccaagatgccagaaaggcagtctcccaaggacccaccatgcagaagtgtcaataaaccacaagttctgaa

SEQ ID NO: 6 - DNA, 4689 bp, Equus caballus
cggccagggacgggaagtgggcggggccggccagagcagcgagctgggcgcggagccgccgagtggcagggtgaagactaactggaaccctggcggcaggccccccgggatgctctgggcatggcctctggagccctacaggagagcagccagatggcagaggagacactgggcttcctggacatgctcctctgcgactttccagccccactgagcccagacagccctctgccgtggaaggtgccagggacagtgctgaggcaggaggaggtggaaggcgagctggccgagctggcgatgggtttcctgggcagcaggaatgctccgccaccacttgcttcgtgtctggcccatgaggcagtttccaagctgctgcaggcggacctttccgaattcaggaagaagcccaggcaggaggaggatgacgacgcagaagaggagaaggcccctgtgaccttgctggatgctgagggcctggtgaggactttctttaaccagctctgggaagtatgcagccggtggcagaagcaggtgccctcgactgcccaggctccgcagaggcagtggctggtctccatccacgccatccggaacactcgccgcaagatggaggaccggcacgtgtgcctttcggccttcaaccagctcttcggcctgtccgaccccgtggaccgcgcctactttgccgtgtttgacggtcacggcggggtggacgctgcgaggtacgctgctgcacacgtgcatgcccatgctgcccgccggccggagctacctacagaccctgcaggggccctcagggaagccttccggcgcaccgacgagatgtttctgtggaaagccaagcgagagcggctgcagagcggcaccaccggcgtgtgcgcgctcatcgcgggaaagaccctgcacgtcgcctggctcggagactctcaggtcatcctggtgcagcagggacaggtggtgaagctgatggagccgcacagacccgagcgacaggacgagcgggagcgcatcgaggcgctgggtggcttcgtgtctcacatggactgctggagagtcaacgggaccctggctgtctccagagccatcggggatgtcttccagaagccctacgtgtctggggaggcggatgcagcctcccaggagctgacgggctccgaggactacctgctgctcgcctgcgacggctttttcgacgttgtcccccaccatgaggttgctggcctcgtgcagagccacctggtcaggcagcagggcagtgggctacacgtcgccgaggagctggtggctgcagcccgggagcggggctcccacgacaacatcacagtcatggtggtcttcctcagggacccccgagccctgctggagggcggggcccagggggcaggggacttgccctctggcctctcagagccagagaccaacacaccaccgagaagctaggaggtcccagcccctggccccaccctgtgaccctccgtcagatgccttaggacctgatggctagcagtgcaggtggcaccgcccgcgcagtgctttgcccagcccccgagcccctcgtgttgcttgcattggcccatcccgggggatgggatctgcactgggtggtgagtgtcgcgccctgctggtggtaggcagtgggacggcaggatctccaagggagcctaggaagagacctcaccacggagccaaggccaggaggtgggccggcccttgctcccggtcaggcaggggctggaccagaggccacaggcgccaggcgagcactcagggcagcaggacacagcatggggggccatcgggcagacctgggagccacggacattttcaagagcgtcctgggtgcccggcacaggtttctcactggctccacgctgcccacagccgcgagggctcagatccgcagatgcgcctggcgggtccctgctggccgagccggcttccccaccaggaaaaaagggagcaccaggcatctggtctgctgcatcgcccaccactggtgacacgcctgccctggcctccctggtcccccatccctggagcgcccggggaggggggtctcagaatgaccatgcgcgcctttcatttgcggtcttttgtcagggagcagtcagattcagaagcagtatttcagggctgtctttttcgtcagtgccaaggtacctgatgtgtcatgtaacttagacagggcttagtgtttggtttatcccttaggaaactaagtattgattttttttttttttaggggaaacgtcttttgcagtattactgaattttttttcccctaaatcaggactgagacaaaatttttccaggtggtgttaataagccattcagtgccttaaacagcctaaggtgaggctggaagtgccgggcctctgacggattccgccaagcatgttgcagttcttacaaaggatttattttgtcagagtggcatgattcgggctagagttcttcctggaagagttctgtctttgtgacgtgccactgactcccgtgtggagttgaaaggggcagggctgggcgagggctccccgcagaggccagggccactggagagaggagcctgggggcccgactcgcggtggtgggcagggctgtgcaggcagagaccagcacatctgggaagggcagtgggtggctttggcttagctgaggcttcaggagaatgtcacggggtgggggggggcagggggaagtgggaggaagcttggcgtcaggtcgccaaggccatgaggtcctgtggttcagagtccccccactgtagtggggccccagggagcaatcccagctgcactgaggggaacctcggcccttctcacccagggaaggggtggatggtgagagccgcccctgctgaggccagtccaccatccagcaagaaaggaggagcttgcagcctcagggctcacccccactcccacggcagggaagcagcagctgagacggacggtcagtgccttgctccagggctcgtgacagaaagcatctcagacatggtcactgcaactcagagagggtttggtggccaacatgaccaggtattgctccatttaacttgaaggcccctctgccttctgggggcccgaagcctcctcgtgagcccctcccagcaccaggctggcacgaggggggatgtggccccacccttcactaccagcccagccacccatgggcatgaccttaatggaaggaggggtcaatggtttctttgcgagtctcactcggttgacagctaccaagagtggagcttcaccccatggagtctgggtcatgttcatgctccggcccaaggcccaaagcacaggagatctctccatgcttgtcccagtgtctcccccaccctgagccaggctgagtcaaaggctcccgccccccacccaccaggctgcctggcagaggccttggctgtctggtgcagcctgggagctgctggtccttgccttgagaccaagggagtccccgctctcccgctgccccgggcctgacctggtgcgtgcacccaggcacggaggaggtctcttccatcttgacagagcccctgggcaccttcaccctctgcgagtcctggcccacttcctctccaggccacaggcactgctctgtaagccttgatccctttctcaagtcctagctttgccactagctcatttttggggctgagcaagccgttgcccctccctgggcctcagtttccccttttagggcacagggttgcactgagtctgtgcagcagctcctggaacttgggaggtgcaggggctgcttgcggtgagcctggtgcagcaggggagctactgccggtgctgaggccccgcccaccgccagcagggaggggtgattaagtactattggtgtctattcttaaatttaagtgggttggaaaagaatcgctgcagatgccagcacctcacgtgggtgcaaaagctggagagccggccctgccttcacaggtagggctgatgctgagcagcgggggtggcagaagcgtaggctccctcctcaggcgtggggggcctgggaggggcagggcgccgccgggcccccaggggcttctagtcaagctcccaacctgctgacaccctggaggcaaacgcgtggcctccctgacccagactgtccttggagtccaggactaggaaatggatgtgttatggggggagagtgacaggcgggccccacgggagaagcacaggctctctgaggtccgcggtcatttctgacgacccctgcccttggccgcagtcctgcctccccgccctccctgggccggcacgcggcccccggcctcgccccttcccctgcgtcccggctacctcagcagtattcccagggctggggggcagctctggctgttcctggacctggcccccacccgagctaaacggaaaccgggtgggcctgtgcaaaataagctgttttggtttgattttggctgtagcccaagggggcagccccaggaggcctgcgctttagctttgccaaacgtgccacgccggcctcgccaagggtttatccgcacatctcagtgggttcctgggagtggtgcaaggggggtcttgttctgagacaggatggcggtccccctgtccacagggacaagtcctagcgtgagggggccagaagggcagtctcctcccagggccactgtgctcaagtgtcaataaaccacaagttgtgaaaccctgt

SEQ ID NO: 7 - DNA, 5208 bp, Canis lupus
ggggcggggaggcccggggcgcggggccggtggcctctctgaggcgtgcagcgagtgaataccacctggaatcccaacagagacccccggcatgctctgggcatggcctctggagccccaccgcagagcagccacacggcagaggagatcccaggcttcctggacgccttcctctgcgactttccagccccgctgagcctggagccccctttgccatggaagctcccgggacctgtgctgagccaggaggaggtggaaggcgagctgaccgagctggcgatgggcttcctgagcaacaggagcgctccacctccacttgctgcatctctggcccatgaggcagtttcccagctgctacagaccgacctttctgaattcaggaagttgcccaggcaggaggaggaagaagatgacgatgaggaagagaaggcccctgtgaccttgctggatgccaagggcctggcacgaagctgctttaaccagctctgggaagtatgcagccagtggcagaagcaggtgccctcaactgcccaggttcctcagcggcagtggctggtctccatgcatgccatccggaatacacgccgcaagatggaggaccggcatgtgtgccttcccgccttcaatcagctcttcggcctgtcggaccccgtggaccgagcctactttgccgtgttcgatggtcacggaggggtggacgctgcacagtacgccgccgtgcacgtgcacaccaatctggcccgccagccggagctgctcacggaccccgcgggagccctcagagaagccttccggcacaccgatgagatgtttctctggaaagccaagcgagagcggctgcagagcggcactacaggggtgtgcgcactcatcgtgggaaagaccctgcacatcgcctggcttggggactcccaggtcatcctggtgcagcagggacaggtggtgaagctgatggagcctcacaggcctgagcgacaggatgagaaggagcgcattgaggcgctgggcggcttcgtgtcccacatggactgctggagagtcaacgggaccctggccgtgtccagagccatcggggacgtgttccagaagccctacgtgtcaggggaggcggactcggcctctcgggagctgacgggctccgaggactacctgctgctggcctgcgacggcttcttcgacgtcgtcccccaccaggaggtcgcgggcctcgtccacagccacctggcccggcagcagggcagcgggctgcaggttgccgaggagctggtggccgcggcccgggagcggggctcccacgacaacatcacggtcatggtggtcttcctcagggacccccgagacctactgaagggcggggcccaggggacaggggatgtgccctctggcctctcacagccagagaccagcactccgcagagcagctaggaggtgtaggccccctgcccccacccgcacccctcccctcagatgccttaggacccgacaggcggtggcgggcaggcgggtgccatcctcagtgcttccccagggccccgaaccccctgctcgcatcagtccatcctggtgtctggggaactgcactgggtggtggtgttattcatgccccgctcgggcaggcagtggggtggcctggatccccaaaggaggcctagggaagagacctcaccaaagagaagatgacccaagaagtggagcagctcttgctcccagccccactgggtaggggcagggccagaggccacaggcgcgggccacagccagaccaaagacactgggcctgtgcccggggcagcaggatgctgcacgtgagtccccgggcagacccaggagcgacggacatttccaagcgtgtcctgggcgcctggctcaggcttctcgtttgctcctcactggccacggctgggaggccccagtctctacagatgggcctggctgggcctggcctgccaagctggcttcccgagtggggagagcggcccctggggagggaggcgcaccagggatctggtctgcagtgggtgctcacggcccccagcctccctgcccccctgcccccccatcccgcgcacacccagggagggggaggtcagaacgatgacacgtgtgtctttttatttgtgggctttttcccccccagggaagtctaactcagaagcagtatttcaggtttttgcctttgttttgtcagtgccaagttgacctgttgtgtcatataacttaagcagagcttagcatttattttattcttagaaaacttaagtattgattttttttttttttgaagaaacttcttttgcagtattactgaatttttttttcctaaatcaggattgaaacaaacacttttccaggtggtgttaataagccattcaagtgccttaaacagctttaggtaaggctagacccgccgggcctgggacggattctaataggcatgcttcagttttttacaaaagcaggatttattttgttctagtggcatgattttggctagaattcttcccggacgagttctttccacttgtcatgattccattcgctgctggaagttagcaaagcagcccctaagagcgtaggctgtgctgtgctacctgggccagaccacgggcctccgggagctccgggtgaccatggccccgccagcctccctccacaccttggccttgctgctcatgtctcatgtctcatgtctctggttggtgcccaggctgagggactgagctccttgaagcgggttcctcgtttgactggaatgttctagaatgtcttgtgttagtaatcgagtcggtttcattgggaaacacgctgaagaggccttggcaagtccgaagcaggctggggggctttggcttggtttggagcctgtcccgctcacctgccccacagcccccaaaaccctcctgtctcccgctcgccatggctgtcaccctctgccacctggctctgcccacagcctaactgcctcagggagggctccacattccctagcatgctctgggcaggtgaggcttgggagccacgaaaccccaggtgcctggctgctcacatccagtgaggcggatatggcatgtgtgtcaagttggaagtggcaggttaggtggagtccaggcagaagtcaagggccatgagagagggatctctggtgggaggacccaggcctacaggcctgggaagagcattaggtagctgagctgaggcttctagagaatgtcgctgggagatgggcagcaggagggagcttggcaccagcccccaggccaggaggtccgcagagtccacccgcagtagccaggccccagcaaagagagcgcagggaggaccctgatcgcgtgcacagagggagccctgctgacgaggaagcgggcagttgggagcctcgggggccctcagcccagggaagggcagtgggtgagatgcccccctccaggccccagacaagcggaccctgacacaggaagggagggagccagctcgggcaggcagtggggcagtgaggtgccacccgcaggctcggcgcttacccctcactcccacagcagggatagagctgctcggatgatggggtcagtgccgtggaaatgatgggaagtaccttaaacaccacaattccaagaaggtttggtaggagccaacagtgaccttgtgttgccccacttaaccggaaggccctgctgctctccgggggtccaaagcccttcttcggcccttcccggcccagggccggcaggaggtggcacgtggccctgcccttcaccaccagcctggctgctcctgggcatgatcttagcagatggaggaagcacgagcttctgtttttctcatttctttgctaatcttattccgtgattgccatcaggagtgcggcttcacccgtggggcccggatcccccacgtgatggtctgactcctgcccgggctcgaggctctcccaggagatctctccaggcttgtcccagtgcccaccccgcctctggcagaggcctctgctgtctggtgctgcccatccttgccttggggctgagggcgcccttgttcatgctgcacagacgctggctttgtgcaagcacctaggcacagaggcctcttccctcttgaccaggggctggtcagccttgaaagtcctggccccccttctcttcaggccacagtccgaggagggcccctagccgggccccccttcccaaccctcgccttgccactagctcattgtgtgagctgagcaagtcctggagccttcctgggcctcagtttctcctttagagggtacagggttgcactggatttgtgtggcagatcctggaattctggaggttcaggggccagcctggtgcatcctggggagccgcggccagtgccgcgggagcccgcccccccccccgccccgaccgccagcagggaggggtgatgaagtattattagtgtctattcttgcagctaagtgggttggaagagaaaggagccgcagacgctggcgccgtatgtggatcacaagctggggcccaggtggagctgagcccaggcagcggcaggggggcagcaggtgccaggcccggggacaaggcagggctcggccgcgccctccgcgcccagtcgtgctgccagcctgctgccgtgcggggccgggtgcgcggcctccccgacctcgcttgtcctcgcgcccaggactaggaaaggggtgcgctgcaggggggacggtggcgggcccaggcccccctggagaggcccggagccagccttgggccgcagccccgtgtcccgcccccctccctcgtgggtgccccggctacctcagcagtattccagggctcgggcgcggcccgggcgggtctaggcctggccctgacgggggctgcaccgaagcccaatgggcccgtgcaaaataagctggtttgggtttgcttctgcctgtagcccacgggaggcagccccaggaggcccgcgcttctcgtcgaggctccacagctttgccaaatgcgccgagctttgccattcagtgcgccgtgccagccttgccaagggttcatctctgcacgtctcagcagggctcctgggagaggtgttctctgcaaggggcgtcttgttccaagacgggatggccattgcccttgtctgcagggccaagtcccaggatgtgggggcagaaactcagtctcctcccaggggtcactgtgctcaagtgtcaataaaccacgagttttgaaaccctga

SEQ ID NO: 8 - DNA, 5313 bp, Felis catus
gcccgccgtccgactgcgcctgcgcggagcccgcggcggcgggggacgggaagtgggcggggtcggccaggagcggctcgcggggtccggagtggcggagtagcagagtgaagatcacctggaaccccggcagagcccccccgggatgctgtggacatggcctctggagacccacagcagagcagccaaatggcagaggagatcccgggcttcctggatgccttcctccatgacttcccagccccactgagcccagagagccctttgccatggaaggtcccaggaacagtgctgagtcaggaggaggtggagggtgagctggccgagctggcgatgggcttcctgagcagcaggaatgctcccccaccacttgcttcatgtctggcccacgaagcggtttcccagctgctgcagatggacctttctgaattcaggaaattgcccagacaggaggaggaggaggaggaggaggaggaagatgacaacgaggaagagaaggcccctgtgaccctgctggatgccaagggcctggcgcgaagtttctttaaccagctctgggaagtatgcagccagtggcagaagcaggtgccctcgagtgcccgggttcctcagcggcagtggctggtctccatccatgccatccggaatactcgccgcaaaatggaggaccggcacgtgtgccttcctgccttcaaccagctctttggcctgtctgaccccgtggaccgagcctactttgctgtgtttgatggccatggaggggtggacgctgcaaggtatgctgctgtacatgtgcacgccaacgtggcccaccggccggagctgcccacagaccccgcgggagccctcagagaagccttccggcacacagatgagatgttcctctggaaagccaagcgagagcggctgcagagtggcaccacgggtgtgtgcgctttcattgcgggaaagaccctgcatgttgcctggctcggggactcccaggtcatcctggtgcaacagggacaggtggtgaagctgatggagccgcacagacctgagcgacaggatgagaaggagcgtattgaagcgctgggcggctttgtgtctcacatggactgctggagagtcaacgggaccctggctgtgtctagagccattggggatgtctttcagaagccctacgtgtcaggagaggccgactcagcctcccgggagctgacaggctctgaggactacctgctgctggcctgtgatggcttcttcgatgtcgtcccccaccaggaggtcgcgggcctcgtccagagccacttggtcagggagcagggcagcgggctgcaggttgctgaggagctggtggctgcagcccgggagcggggctcccacgataacatcacagtcatggtggtcttcctcagggacccccaagacctgctgaagggcagggcccagggggtaggagacgtgcccactggcctcgcagagccagggaccaatgctccacagagacgctaggaggtgcaggctccctgcccctaccccgtaaccctccccctcagatgccttaggacccgacaggtgatggtggacagtgggtgccaccctcacagtgctttcccagggccccaagtctcccgtgctgcttgcattggcccatcctggtggctgtggaactggactgggtcgtgggggatgtgccctgctcggacaggcagtgggatggccttgttctttgaaggaggcctagggaagagacctcaccaaagaaaagacgacccaagaagtggagcagctcttgctcccagccccatcaggtagggggctcaggtggggaccacagccagaccaaagatgctgggcatgtgcatggggcagcaggatgctgcatgagcccccaggcagacccaggagccacggacatttccaagtgcaacctgggtgtccagcacaggtttctcacttgctcctcactggccgccactgggaggctcgtctccgctgacgcacctggcctcccgagccagcttcccaaattggggagaacagagcaccagggacctggtctgcagtgaatgctcacagcccccagcctccctggttccctcccgtcccgcacacacccagtgagaggaaggtcagaatgacaacgaggtgtgtgtcttcagtttgtggtcttttttttttttttccaggacagtcgaactcagaagcagtatttcaggtttttgtctttgttttgtcagtgccaaggtgacctgttgtgtcatgtaacttaagcagagcttagcatttattttattcttggaaaccttaagtattgattttttttttttggaagaaacttcttttgcagtattactgaatttttttcctaaatcaggattgaagcaaacacttttccaggtggtgttaataagccattcaagtgccttaaacagctttaggtgaggctagaaccgccgggcctgggatggattctactaggcatgtttaagtttttacaagagcaggatttattttgttatagtggcatgatttcagctagaattcttcccactccacttcctttctgcccctttgtgatgcgatttgctgctgacagtcagcaaagcagctgaaagagcatgagctatgctgcacagcctgggccagacctaggggctctgggagttcagggtgaccatggcacctcccccagcctccctatgcacctcagtcctgctgcccatgtctctggctggtccccaggctgaggaactgagctccatgagcaggcttctcatttgactggaatgtcctagaacgtctcgtgttggtaatcgaattggtttccttgggaaacacacttacgaggccttggcaagtctggaggcagcctggggggttctggctctgtttggagtctgtccatccacctgccccacagctccccaaacccctcctcctgtttcttgcccaccatggcggcaccctctgttaccttgctttgcccacagcctgaccacctgggggagagctctacgttccctagcatgccctgggcaggtgaggctcagaagccacgaagcttgggtgactcaggctactcacatccagtgaggcagatatagcatgtgtgttgagctagaagtggcagggctgggtgaagagtctggcagagaggggtctctgtgggaggacccaggcctacaggtctgggaagagcggtgggtggctttggaggctcgtggagaatgtcactgggaggtgggcagcaggaggaagcttggcactgtgtcccccaggccagaatccacctgcagtagcaaggctccaggaaggtgggagctgggagagccttggcggcactcatcagcccggggaagggcagacttggtaagagccaccccactgaggcccgagggctgtgcagcagggagggtgggagccagctctgggcaggcaggggggcaatgggggatgccgcccacaggctcagcactcacccctactctcaccgcagggatgaagaagccaagatgatgtggtcagtgccatgctcaagggctcgtgacagaaaaagtatcttaaatgtggtcaccgcagttcagagaaggtttggtgggagccaacaatgacctggtattgccgcacttaatctgaaggccctgctgccttctgggggccagaagccccctctttggcccttcccagcgcagggccggcaagaggtggcatgtggcccctaccagcctggctgcccctgagcatgaccttaacagaggaaagaggtgtgggcttgtttttctcatttctttgctaatcttattctgggattgccatcaagagtggggtttcacctttggagtctggactccccacgtggtggtctgattcctgcccagactcaaggctccaccaggagatctctccaggcttgtcccagtgcccacccctctcgtctgtgagaggcccttgccatctggtgctgctcagccttgccttgaggcggagggagcccttgttcccactgcgcagatgctggctttgtgcaagcacctaggcatagaggtctcttccctcttgaccagggaggactgggagccactgggcatcttcagcctctgaaagtcctagcccacctccttgtcaggccacagtcccaagaaggcctctagccaggaccccaactctagaagcctcagtccccttctcaaatccccactttgccactagctcattatatgagctgagcaagtcgttgaacctccttgggcccccttttctcctttataggacacagggttgcacggaatctgtgcagcagatcttggaattttggaggttcggggcccatcgagggcaagcctggcacatcctggagagctgctaccagtgctaagggaaccctccccctctgctagtgggaggggtggttaagtatcattagtgtctattcttacaattaagtgggttggaaaggaatggagctacagttgctagccgtatatggaacacagaagctggagagagggagccctgcttttacaggtagagctgatcccaagccagcaggaggaggagaggcagacagactcagctgcctccctctgcaggtgtgaggctgaagggcagggcacagccagcgtctcccttgccttttagtcaagctcccaacctgctgaaatccagaggcagatgcatggcctccctgacccagcttgtcctcagcatccaggactaggaaaggggtgtgctttactggggacagtgatgggggcaggcctcactggggaagcacaggccctctgaggtcctgagccagcctttggccacagtcctatctccccctaccctttgaaaccagcatgtggccctcgtccctacccctataatgccagttctgagttccggcttgggtgtctggccacttcagcagtagtcccagggcttgggtcacagctctggcagttcctaggcctggccccgacaagagccaaatgggcaccttaagggcctgtgcaaaataagctgtttttggtttaattctgcctgtagcccaaaggaggcagccccaggaggcccacgcttttcgccgaggctccacagctttgccaaatgcgccgagctttgccattcacatgccatgccagccttgccaagggtttatttgtgcacgtctcagcagggctcctgggagaggtgttctctgcaaggtgcatcttgttccaagatgggactactatttcccctgtgtgcaggggcaagtcccaggagaagagggccagaaacacagtctcttcccaggggtcactgtgctcaagtgtcaataaaccacgagttttgaaaccctgt

SEQ ID NO: 9 - RNA, 25 nt, Artificial Sequence
  siRNA 1
ccggugauguagacaauaauuaaca

SEQ ID NO: 10 - DNA, 23 bp, Artificial Sequence
  siRNA 2
gcaagagaggaagguauucagga

SEQ ID NO: 11 - DNA, 26 bp, Artificial Sequence
  siRNA 3
gccuuaauuccuugcaauagucuctc

SEQ ID NO: 12 - DNA, 25 bp, Artificial Sequence
  siRNA 4
guugagaccaugcagucaaugcatt

SEQ ID NO: 13 - DNA, 25 bp, Artificial Sequence
  siRNA 5
agaccuuuccgaauucaggaagutg

SEQ ID NO: 14 - RNA, 25 nt, Artificial Sequence
  siRNA 6
caccaagaagcuaggugguuuccag

SEQ ID NO: 15 - RNA, 22 nt, Homo sapiens
gcugggauuacaggcaugagcc

SEQ ID NO: 16 - RNA, 99 nt, Homo sapiens
cgcccaccucagccucccaaaaugcugggauuacaggcaugagccacugcggucgaccaugaccuggacauguuugugcccaguacugucaguuugcag

SEQ ID NO: 17 - RNA, 22 nt, Homo sapiens
uuuagagacggggucuugcucu

SEQ ID NO: 18 - RNA, 86 nt, Homo sapiens
ggcugggcaacauagcgagaccucaacucuacaauuuuuuuuuuuuuaaauuuuagagacggggucuugcucuguugccaggcuuu

SEQ ID NO: 19 - RNA, 17 nt, Homo sapiens
cuccgggacggcugggc

SEQ ID NO: 20 - RNA, 89 nt, Homo sapiens
accuccgggacggcugggcgccggcggccgggagauccgcgcuuccugaaucccggccggcccgcccggcgcccguccgcccgcggguc

SEQ ID NO: 21 - DNA, 3818 bp, Homo sapiens
ctccccggtaaagtctcgcggtgctgccgggctcagccccgtctcctcctcttgctccctcggccgggcggcggtgactgtgcaccgacgtcggcgcgggctgcaccgccgcgtccgcccgcccgccagcatggccaccaccgccacctgcacccgtttcaccgacgactaccagctcttcgaggagcttggcaagggtgctttctctgtggtccgcaggtgtgtgaagaaaacctccacgcaggagtacgcagcaaaaatcatcaataccaagaagttgtctgcccgggatcaccagaaactagaacgtgaggctcggatatgtcgacttctgaaacatccaaacatcgtgcgcctccatgacagtatttctgaagaagggtttcactacctcgtgtttgaccttgttaccggcggggagctgtttgaagacattgtggccagagagtactacagtgaagcagatgccagccactgtatacatcagattctggagagtgttaaccacatccaccagcatgacatcgtccacagggacctgaagcctgagaacctgctgctggcgagtaaatgcaagggtgccgccgtcaagctggctgattttggcctagccatcgaagtacagggagagcagcaggcttggtttggttttgctggcaccccaggttacttgtcccctgaggtcttgaggaaagatccctatggaaaacctgtggatatctgggcctgcggggtcatcctgtatatcctcctggtgggctatcctcccttctgggatgaggatcagcacaagctgtatcagcagatcaaggctggagcctatgatttcccatcaccagaatgggacacggtaactcctgaagccaagaacttgatcaaccagatgctgaccataaacccagcaaagcgcatcacggctgaccaggctctcaagcacccgtgggtctgtcaacgatccacggtggcatccatgatgcatcgtcaggagactgtggagtgtttgcgcaagttcaatgcccggagaaaactgaagggtgccatcctcacgaccatgcttgtctccaggaacttctcagctgccaaaagcctattgaacaagaagtcggatggcggtgtcaagccacagagcaacaacaaaaacagtctcgtaagcccagcccaagagcccgcgcccttgcagacggccatggagccacaaaccactgtggtacacaacgctacagatgggatcaagggctccacagagagctgcaacaccaccacagaagatgaggacctcaaagctgccccgctccgcactgggaatggcagctcggtgcctgaaggacggagctcccgggacagaacagccccctctgcaggcatgcagccccagccttctctctgctcctcagccatgcgaaaacaggagatcattaagattacagaacagctgattgaagccatcaacaatggggactttgaggcctacacgaagatttgtgatccaggcctcacttcctttgagcctgaggcccttggtaacctcgtggaggggatggatttccataagttttactttgagaatctcctgtccaagaacagcaagcctatccataccaccatcctaaacccacacgtccacgtgattggggaggacgcagcgtgcatcgcctacatccgcctcacccagtacatcgacgggcagggtcggcctcgcaccagccagtcagaagagacccgggtctggcaccgtcgggatggcaagtggctcaatgtccactatcactgctcaggggcccctgccgcaccgctgcagtgagctcagccacaggggctttaggagattccagccggaggtccaaccttcgcagccagtggctctggagggcctgagtgacagcggcagtcctgtttgtttgaggtttaaaacaattcaattacaaaagcggcagcagccaatgcacgcccctgcatgcagccctcccgcccgcccttcgtgtctgtctctgctgtaccgaggtgttttttacatttaagaaaaaaaaaaaagaaaaaaagattgtttaaaaaaaaaaggaatccataccatgatgcgttttaaaaccaccgacagcccttgggttggcaagaaggcaggagtatgtatgaggtccatcctggcatgagcagtggctcacccaccggccttgaagaggtgagcttggcctctctggtccccatggacttagggggaccaggcaagaactctgacagagctttgggggccgtgatgtgattgcagctcctgaggtggcctgcttaccccaggtctaggaatgaacttctttggaacttgcataggcgcctagaatggggctgatgagaacatcgtgaccatcagacctacttgggagagaacgcagagctcccagcctgctgtggaggcagctgagaagtggtggcctcaggactgagagcccggacgttgctgtactgtcttgtttagtgtagaagggaagagaattggtgctgcagaagtgtacccgccatgaagccgatgagaaacctcgtgttagtctgacatgcactcactcatccatttctataggatgcacaatgcatgtgggccctaatattgaggccttatccctgcagctaggagggggaggggttgttgctgctttgcttcgtgttttcttctaacctggcaaggagagagccaggccctggtcagggctcccgtgccgcctttggcggttctgtttctgtgctgatctggaccatctttgtcttgccttttcacggtagtggtccccatgctgaccctcatctgggcctgggccctctgccaagtgcccctgtgggatgggaggagtgaggcagtgggagaagaggtggtggtcgtttctatgcattcaggctgcctttggggctgcctcccttcttattcttccttgctgcacgtccatctcttttcctgtctttgagattgacctgactgctctggcaagaagaagaggtgtccttacagaggcctctttactgaccaactgaagtatagacttactgctggacaatctgcatgggcatcacccctccccgcatgtaacccaaaagaggtgtccagagccaaggcttctaccttcattgtccctctctgtgctcaaggagttccattccaggaggaagagatctataccctaagcagatagcaaagaagataatggaggagcaattggtcatggccttggtttccctcaaaacaacgctgcagatttatctgcacaaacatctccacttttgggggaaaggtgggtagattccagttccctggactaccttcaggaggcacgagagctgggagaagaggcaaagctacaggtttacttgggagccagctgagaagagagcagactcacaggtgctggtgcttggatttagccaggctcctccgagcacctcatgcatgtcccagcccctgggccctagccctttcctgccctgcagtctgcagtgccagcacgcaaatcccttcaccacagggtttcgttttgctggcttgaagacaaatggtcttagaattcattgagacccatagcttcatatggctgctccagccccacttcttagcattcttactcctcttctggggctaatgtcagcatctatagacaatagactattaaaaaatcaccttttaaacaagaaacggaaggcatttgatgcagaatttttgcatgacaacatagaaataatttaaaaatagtgtttgttctgaatgttggtagacccttcatagctttgttacaatgaaaccttgaactgaaaatatttaataaaataacctttaaacagtc

SEQ ID NO: 22 - protein, 391 aa, Homo sapiens
MDLEKNYPTPRTSRTGHGGVNQLGGVFVNGRPLPDVVRQRIVELAHQGVRPCDISRQLRVSHGCVSKILGRYYETGSIKPGVIGGSKPKVATPKVVEKIAEYKRQNPTMFAWEIRDRLLAERVCDNDTVPSVSSINRIIRTKVQQPPNQPVPASSHSIVSTGSVTQVSSVSTDSAGSSYSISGILGITSPSADTNKRKRDEGIQESPVPNGHSLPGRDFLRKQMRGDLFTQQQLEVLDRVFERQHYSDIFTTTEPIKPEQTTEYSAMASLAGGLDDMKANLASPTPADIGSSVPGPQSYPIVTGRDLASTTLPGYPPHVPPAGQGSYSAPTLTGMVPGSEFSGSPYSHPQYSSYNDSWRFPNPGLLGSPYYYSAAARGAAPPAAATAYDRH

SEQ ID NO: 23 - protein, 391 aa, Equus caballus
MDLEKTYPTPRTGRTGHGGVNQLGGVFVNGRPLPDVVRQRIVELAHQGVRPCDISRQLRVSHGCVSKILGRYYETGSIKPGVIGGSKPKVATPKVVEKIAEYKRQNPTMFAWEIRDRLLAERVCDNDTVPSVSSINRIIRTKVQQPPNQPVPASSHSIVSTGSVTQVSSVSTDSAGSSYSISGILGITSPSADTNKRKRDEGVPESPVPNGHSLPGRDFLRKQMRGDLFTQQQLEVLDRVFERQHYADIFTTTEPIKPEQTTEYSAMASLAGGLDDMKANLTSPTPADIGSSVPGPQSYPIVTGRDLASTTLPGYPPHVPPAGQGSYSAPTLTGMVPGSEFSGSPYSHPQYPSYNDSWRFPNPGLLGSPYYYSAAARGAAPPAAATAYDRH

SEQ ID NO: 24 - protein, 391 aa, Canis lupus
MDLEKNYPTPRSGRTGHGGVNQLGGVFVNGRPLPDVVRQRIVELAHQGVRPCDISRQLRVSHGCVSKILGRYYETGSIKPGVIGGSKPKVATPKVVEKIAEYKRQNPTMFAWEIRDRLLAERVCDNDTVPSVSSINRIIRTKVQQPPNQPVPASSHSIVSTGSVTQVSSVSTDSAGSSYSISGILGITSPSADTNKRKRDEGIQESPVPNGHSLPGRDFLRKQMRGDLFTQQQLEVLDRVFERQHYSDIFTTTEPIKPEQTTEYSAMASLAGGLDDMKANLTSPTPADIGSSVPGPQSYPIVTGRDLASTTLPGYPPHVPPAGQGSYSAPTLTGMVPGSEFSGSPYSHPQYPSYNDSWRFPNPGLLGSPYYYSAAARGAAPPAAATAYDRH

SEQ ID NO: 25 - protein, 391 aa, Felis catus
MDLEKNYPTPRTGRTGHGGVNQLGGVFVNGRPLPDVVRQRIVELAHQGVRPCDISRQLRVSHGCVSKILGRYYETGSIKPGVIGGSKPKVATPKVVEKIAEYKRQNPTMFAWEIRDRLLAERVCDNDTVPSVSSINRIIRTKVQQPPNQPVPASSHSIVSTGSVTQVSSVSTDSAGSSYSISGILGITSPSADTNKRKRDEGIQESPVPNGHSLPGRDFLRKQMRGELFTQQQLEVLDRVFERQHYSDIFTTTEPIKPEQTTEYSAMASLAGGLDDMKANLTSPTPTDIGSSVPGPQSYPIVTGRDLASTTLPGYPPHVPPAGQGSYSAPTLTGMVPGSEFSGSPYSHPQYPSYNDSWRFPNPGLLGSPYYYSAAARGAAPPAAATAYDRH

SEQ ID NO: 26 - protein, 454 aa, Homo sapiens
MSSGAPQKSSPMASGAEETPGFLDTLLQDFPALLNPEDPLPWKAPGTVLSQEEVEGELAELAMGFLGSRKAPPPLAAALAHEAVSQLLQTDLSEFRKLPREEEEEEEDDDEEEKAPVTLLDAQSLAQSFFNRLWEVAGQWQKQVPLAARASQRQWLVSIHAIRNTRRKMEDRHVSLPSFNQLFGLSDPVNRAYFAVFDGHGGVDAARYAAVHVHTNAARQPELPTDPEGALREAFRRTDQMFLRKAKRERLQSGTTGVCALIAGATLHVAWLGDSQVILVQQGQVVKLMEPHRPERQDEKARIEALGGFVSHMDCWRVNGTLAVSRAIGDVFQKPYVSGEADAASRALTGSEDYLLLACDGFFDVVPHQEVVGLVQSHLTRQQGSGLRVAEELVAAARERGSHDNITVMVVFLRDPQELLEGGNQGEGDPQAEGRRQDLPSSLPEPETQAPPRS

SEQ ID NO: 27 - protein, 439 aa, Equus caballus
MASGALQESSQMAEETLGFLDMLLCDFPAPLSPDSPLPWKVPGTVLRQEEVEGELAELAMGFLGSRNAPPPLASCLAHEAVSKLLQADLSEFRKKPRQEEDDDAEEEKAPVTLLDAEGLVRTFFNQLWEVCSRWQKQVPSTAQAPQRQWLVSIHAIRNTRRKMEDRHVCLSAFNQLFGLSDPVDRAYFAVFDGHGGVDAARYAAAHVHAHAARRPELPTDPAGALREAFRRTDEMFLWKAKRERLQSGTTGVCALIAGKTLHVAWLGDSQVILVQQGQVVKLMEPHRPERQDERERIEALGGFVSHMDCWRVNGTLAVSRAIGDVFQKPYVSGEADAASQELTGSEDYLLLACDGFFDVVPHHEVAGLVQSHLVRQQGSGLHVAEELVAAARERGSHDNITVMVVFLRDPRALLEGGAQGAGDLPSGLSEPETNTPPRS

SEQ ID NO: 28 - protein, 440 aa, Canis lupus
MASGAPPQSSHTAEEIPGFLDAFLCDFPAPLSLEPPLPWKLPGPVLSQEEVEGELTELAMGFLSNRSAPPPLAASLAHEAVSQLLQTDLSEFRKLPRQEEEEDDDEEEKAPVTLLDAKGLARSCFNQLWEVCSQWQKQVPSTAQVPQRQWLVSMHAIRNTRRKMEDRHVCLPAFNQLFGLSDPVDRAYFAVFDGHGGVDAAQYAAVHVHTNLARQPELLTDPAGALREAFRHTDEMFLWKAKRERLQSGTTGVCALIVGKTLHIAWLGDSQVILVQQGQVVKLMEPHRPERQDEKERIEALGGFVSHMDCWRVNGTLAVSRAIGDVFQKPYVSGEADSASRELTGSEDYLLLACDGFFDVVPHQEVAGLVHSHLARQQGSGLQVAEELVAAARERGSHDNITVMVVFLRDPRDLLKGGAQGTGDVPSGLSQPETSTPQSS

SEQ ID NO: 29 - protein, 445 aa, Felis catus
MASGDPQQSSQMAEEIPGFLDAFLHDFPAPLSPESPLPWKVPGTVLSQEEVEGELAELAMGFLSSRNAPPPLASCLAHEAVSQLLQMDLSEFRKLPRQEEEEEEEEEDDNEEEKAPVTLLDAKGLARSFFNQLWEVCSQWQKQVPSSARVPQRQWLVSIHAIRNTRRKMEDRHVCLPAFNQLFGLSDPVDRAYFAVFDGHGGVDAARYAAVHVHANVAHRPELPTDPAGALREAFRHTDEMFLWKAKRERLQSGTTGVCAFIAGKTLHVAWLGDSQVILVQQGQVVKLMEPHRPERQDEKERIEALGGFVSHMDCWRVNGTLAVSRAIGDVFQKPYVSGEADSASRELTGSEDYLLLACDGFFDVVPHQEVAGLVQSHLVREQGSGLQVAEELVAAARERGSHDNITVMVVFLRDPQDLLKGRAQGVGDVPTGLAEPGTNAPQRR

SEQ ID NO: 30 - protein, 556 aa, Homo sapiens
MATTATCTRFTDDYQLFEELGKGAFSVVRRCVKKTSTQEYAAKIINTKKLSARDHQKLEREARICRLLKHPNIVRLHDSISEEGFHYLVFDLVTGGELFEDIVAREYYSEADASHCIHQILESVNHIHQHDIVHRDLKPENLLLASKCKGAAVKLADFGLAIEVQGEQQAWFGFAGTPGYLSPEVLRKDPYGKPVDIWACGVILYILLVGYPPFWDEDQHKLYQQIKAGAYDFPSPEWDTVTPEAKNLINQMLTINPAKRITADQALKHPWVCQRSTVASMMHRQETVECLRKFNARRKLKGAILTTMLVSRNFSAAKSLLNKKSDGGVKPQSNNKNSLVSPAQEPAPLQTAMEPQTTVVHNATDGIKGSTESCNTTTEDEDLKAAPLRTGNGSSVPEGRSSRDRTAPSAGMQPQPSLCSSAMRKQEIIKITEQLIEAINNGDFEAYTKICDPGLTSFEPEALGNLVEGMDFHKFYFENLLSKNSKPIHTTILNPHVHVIGEDAACIAYIRLTQYIDGQGRPRTSQSEETRVWHRRDGKWLNVHYHCSGAPAAPLQ

SEQ ID NO: 31 - DNA, 20 bp, Artificial Sequence
  CASP14 forward primer
gttccgaagaagacctggat

SEQ ID NO: 32 - DNA, 20 bp, Artificial Sequence
  CASP14 reverse primer
ttctccagcttgaccatctc

SEQ ID NO: 33 - DNA, 20 bp, Artificial Sequence
  GALNT6 forward primer
ggagcacctaaaggagaagc

SEQ ID NO: 34 - DNA, 20 bp, Artificial Sequence
  GALNT6 reverse primer
ctgtcttgtcctcagcgatt

SEQ ID NO: 35 - DNA, 20 bp, Artificial Sequence
  PAX5 forward primer
catccggacaaaagtacagc

SEQ ID NO: 36 - DNA, 20 bp, Artificial Sequence
  PAX5 reverse primer
accggagactcctgaatacc

SEQ ID NO: 37 - DNA, 20 bp, Artificial Sequence
  PPM1F forward primer
cttggctttcctgagaaaca

SEQ ID NO: 38 - DNA, 20 bp, Artificial Sequence
  PPM1F reverse primer
cttggctttcctgagaaaca

SEQ ID NO: 39 - DNA, 20 bp, Artificial Sequence
  SUMO2 forward primer
atggttctgtggtgcagttt

SEQ ID NO: 40 - DNA, 20 bp, Artificial Sequence
  SUMO2 reverse primer
ctgctgttggaacacatcaa

SEQ ID NO: 41 - DNA, 20 bp, Artificial Sequence
  beta-Actin forward primer
atcctcaccctgaagtaccc

SEQ ID NO: 42 - DNA, 20 bp, Artificial Sequence
  beta-Actin reverse primer
agcctggatagcaacgtaca